(19) United States
(12) Patent Application Publication
(10) Pub. No.: US 2026/0226048 A1
(43) Pub. Date: Aug. 6, 2026

(54) MULTI-CYCLIC IRAK AND FLT3 INHIBITING COMPOUNDS AND USES THEREOF

(71) Applicants: CHILDREN'S HOSPITAL MEDICAL CENTER, Cincinnati, OH (US); THE UNITED STATES OF AMERICA, AS REPRESENTED BY THE SECRETARY, DEPT. OF HEALTH AND HUMAN SERVICES, Bethesda, MD (US); KUROME THERAPEUTICS, INC, Cincinnati, OH (US)

(72) Inventors: Scott Bryan HOYT, Arlington, VA (US); Daniel T. STARCZYNOWSKI, Cincinnati, OH (US); Craig Joseph THOMAS, Gaithersburg, MD (US); Jan Susan ROSENBAUM, Cincinnati, OH (US)

(73) Assignees: CHILDREN'S HOSPITAL MEDICAL CENTER, Cincinnati, OH (US); THE UNITED STATES OF AMERICA, AS REPRESENTED BY THE SECRETARY, DEPT. OF HEALTH AND HUMAN SERVICES, Bethesda, MD (US); KUROME THERAPEUTICS, INC, Cincinnati, OH (US)

(21) Appl. No.: 19/152,632

(22) PCT Filed: Feb. 1, 2024
(86) PCT No.: PCT/US2024/014038

§ 371 Date: Jul. 31, 2025

Related U.S. Application Data
(60) Provisional application No. 63/482,736, filed on Feb. 1, 2023.

(51) Int. Cl. C07D 471/04; A61K 31/506; A61K 31/5377; A61K 31/541; A61K 45/06; A61P 35/02; C07D 487/04

(52) U.S. Cl. CPC C07D 471/04; A61K 31/506; A61K 31/5377; A61K 31/541; A61K 45/06; A61P 35/02; C07D 487/04

(57) ABSTRACT

Some embodiments of the invention include inventive compounds (e.g., compounds of Formula (I), (II), or (III)) and compositions (e.g., pharmaceutical compositions) which inhibit IRAK and/or FLT3 and which can be used for treating, for example, certain diseases. Some embodiments include methods of using the inventive compound (e.g., in compositions or in pharmaceutical compositions) for administering and treating (e.g., diseases such as hematopoietic cancers, myelodysplastic syndromes (MDS), acute myeloid leukemia (AML), etc.). Additional embodiments provide disease treatment using combinations of the inventive IRAK and/or FLT3 inhibiting compounds with other therapies, such as cancer therapies.

### CROSS-REFERENCE TO RELATED APPLICATIONS

This application claims priority to U.S. Provisional Patent Application No. 63/482,736, filed Feb. 1, 2023, which is incorporated herein by reference in its entirety.

### GOVERNMENT RIGHTS

This invention was made in the performance of a Cooperative Research and Development Agreement with the National Institutes of Health, an Agency of the Department of Health and Human Services. The Government of the United States has certain rights in this invention.

### FIELD

The invention disclosed herein generally relates to compounds and compositions which are kinase inhibitors and the use of the same in treating diseases and disorders, including cancers.

### BACKGROUND

Myelodysplastic syndromes (MDS) are malignant, potentially fatal blood diseases that arise from a defective hematopoietic stem/progenitor cell, confer a predisposition to acute myeloid leukemia (AML) (Corey et al., 2007; Nimer, 2008), and often progress to chemotherapy-resistant secondary acute myeloid leukemia (sAML). A majority of patients having MDS die of marrow failure, immune dysfunction, and/or transformation to overt leukemia.

MDS are heterogeneous diseases with few treatment options, as there is a lack of effective medicines capable of providing a durable response. Current treatment options for MDS are limited but include allogeneic HSC transplantation, demethylating agents, and immunomodulatory therapies (Ebert, 2010). While hemopoietic stem cell (HSC) transplantation can be used as a curative treatment for MDS, this option is unavailable to many older patients, who instead receive supportive care and transfusions to ameliorate disease complications. Unfortunately, MDS clones can persist in the marrow even after HSC transplantation, and the disease invariably advances (Tehranchi et al., 2010). For advanced disease or high-risk MDS, patients may also receive immunosuppressive therapy, epigenetic modifying drugs, and/or chemotherapy (Greenberg, 2010). Despite recent progress, most MDS patients exhibit treatment-related toxicities or relapse (Sekeres, 2010a). Overall, the efficacy of these treatments is variable, and generally life expectancies are only slightly improved as compared to supportive care. The complexity and heterogeneity of MDS, and the lack of human xenograft models are obstacles which are challenging for identifying and evaluating novel molecular targets for this disease.

Approximately 30% of MDS patients also develop aggressive AML due to acquisition of additional mutations in the defective hematopoietic stem/progenitor cell (HSPC) (Greenberg et al., 1997). AML is a cancer of the myeloid line of blood cells, characterized by the rapid growth of abnormal white blood cells that accumulate in the bone marrow and interfere with the production of normal blood cells. AML is the most common acute leukemia affecting adults, and its incidence increases with age. Although AML is a relatively rare disease, accounting for approximately 1.2% of cancer deaths in the United States, its incidence is expected to increase as the population ages. Several risk factors and chromosomal abnormalities have been identified, but the specific cause is not clear. As an acute leukemia, AML progresses rapidly and is typically fatal within weeks or months if left untreated. The prognosis for AML that arises from MDS is worse as compared to other types of AML.

Several compounds are known to treat blood disorders and cancers (e.g. MDS, AML), but do so inadequately. While some known compounds, such as Quizartinib, Gilteritinib, and Crenolanib, can be used to treat AML, some of these treatments do not result in complete remission or partial remission. In some instances, for example, treatment can result in adaptive resistance or selecting mutations that are resistant to inhibitors, as with Quizartinib, in particular, where repeated administration can lead to desensitization in tumor cell suppression of proliferation (Melgar et al., 2019).

In treating MDS and/or AML, it is important to develop therapies capable of inhibiting the adaptive resistance mechanism, to improve survival in the context of AML and MDS. There is also an unmet need in AML for drugs that increase overall survival, decrease the length of hospital stay as well as hospital readmission rates, overcome acquired resistance to other treatments, and increase the success rate for hematopoietic stem cell transplant. There is additionally a need for drugs for treating MDS which can slow the conversion rate to AML, and decrease transfusion dependence.

It is therefore necessary to develop treatments and methods of effectively treating MDS and/or AML. Additionally, in doing so, it will be important to determine whether a patient is likely to be responsive to a particular treatment or method of treatment. Certain embodiments of the invention can address one or more of these issues.

### SUMMARY OF THE DISCLOSURE

In one embodiment, the present disclosure provides a compound of formula (I), (II), or (III):

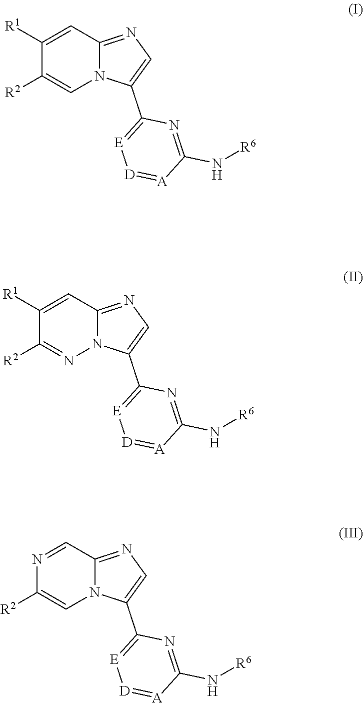

or a salt, ester, solvate, optical isomer, geometric isomer, salt of an isomer, prodrug, or derivative thereof, wherein: A is selected from N and CR⁵; D is selected from N and CR⁴; E is selected from N and CR³; at least one of A, D, and E is N; R¹, R², R³, R⁴, and R⁵ are each independently selected from H, deuterium, halogen, hydroxy, oxo, —CN, —C(═O)H, —C(═O)OH, C₁-C₇ alkyl, C₂-C₇ alkenyl, C₂-C₇ alkynyl, C₁-C₇ alkoxy, —C(═O)NR³¹R³², cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl is optionally substituted with one or more of deuterium, halogen, hydroxy, oxo, —C(═O)H, —C(═O)OH, nitro (—NO₂), —NH₂, —N(CH₃)₂, cyano (—CN), ethynyl (—CCH), propynyl, —SO₃H, cycloalkyl, heterocyclyl, aryl, heteroaryl, pyrrolyl, piperidyl, piperazinyl, morpholinyl, —C(═O)-morpholin-4-yl, —C(═O)NH₂, —C(═O)N(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl; R⁶ is

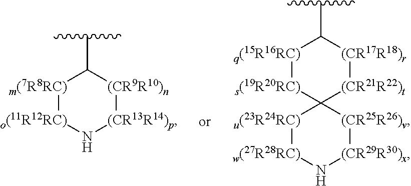

or C₃-C₆ cycloalkyl substituted with one or more —NR³³R³⁴; R⁷, R⁸, R⁹, R¹⁰, R¹¹, R¹², R¹³, and R¹⁴ are each independently selected from H, deuterium, halogen, hydroxy, oxo, —CN, —C(═O)H, —C(═O)OH, C₁-C₇ alkyl, C₂-C₇ alkenyl, C₂-C₇ alkynyl, C₁-C₇ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl are optionally substituted with one or more of deuterium, halogen, hydroxy, oxo, —C(═O)H, —C(═O)OH, nitro (—NO₂), —NH₂, —N(CH₃)₂, cyano (—CN), ethynyl (—CCH), propynyl, —SO₃H, cycloalkyl, heterocyclyl, aryl, heteroaryl, pyrrolyl, piperidyl, piperazinyl, morpholinyl, —C(═O)-morpholin-4-yl, —C(═O)NH₂, —C(═O)N(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl; R¹⁵, R¹⁶, R¹⁷, R¹⁸, R¹⁹, R²⁰, R²¹, R²², R²³, R²⁴, R²⁵, R²⁶, R²⁷, R²⁹, R²⁹, and R³⁰ are independently selected from H, deuterium, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl are optionally substituted with one or more of deuterium, halogen, hydroxy, oxo, —C(═O)H, —C(═O)OH, nitro (—NO₂), —NH₂, —N(CH₃)₂, cyano (—CN), ethynyl (—CCH), propynyl, —SO₃H, cycloalkyl, heterocyclyl, aryl, heteroaryl, pyrrolyl, piperidyl, piperazinyl, morpholinyl, —C(═O)-morpholin-4-yl, —C(═O)NH₂, —C(═O)N(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl; R³¹ and R³² are each independently selected from H, C₁-C₆ alkyl, and C₃-C₆ cycloalkyl, wherein C₁-C₆ alkyl and C₃-C₆ cycloalkyl are optionally substituted with one or more halogen; R³³ and R³⁴ are each independently selected from H and C₁-C₆ alkyl; and m, n, o, p, q, r, s, t, u, v, w, and x are independently selected from 0, 1, 2, 3, 4, or 5, where q+r+s+t is at least 1, and where u+v+w+x is at least 1. In one embodiment, the compound of Formula (I) is a compound of Formula (Ib-5010-WO50):

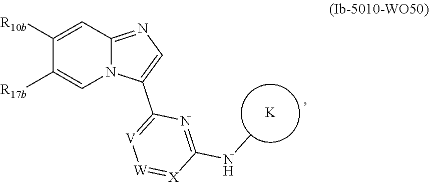

or a salt, ester, solvate, optical isomer, geometric isomer, or salt of an isomer thereof; wherein: V is N or CR₁₁; W is N or CR₁₂; X is N or CR₁₃;

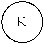

is

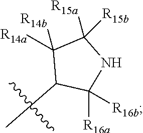

R_{10b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from C₁-C₆ alkyl and halogen; R_{17b} is selected from halogen, C₁-C₆ alkyl, C₁-C₆ alkoxy, C₃-C₆ cycloalkyl, —O—(C₃-C₆ cycloalkyl), C₃-C₉ heterocyclyl, imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from —OH, C₁-C₆ alkyl, and halogen; R₁₁, R₁₂, and R₁₃ are each independently selected from H, C₁-C₆ alkoxy, and halogen; R₁₄ₐ, R_{14b}, R₁₅ₐ, R_{15b}, R₁₆ₐ, R_{16b}, R₁₈ₐ, and R_{18b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and one of V, W, or X is N. In one embodiment, at least one of (i)-(v) applies: (i) each of R_{14b}, R₁₅ₐ, R_{15b}, R₁₆ₐ, and R_{16b} is H and R₁₄ₐ is F; (ii) R₁₁, R₁₂, and R₁₃, if present, are H; (iii) V is CR₁₁ wherein R₁₁ is F, W is CR₁₂ wherein R₁₂ is H, and X is N; (iv) R_{10b} is selected from H and —OCH₃; and (v) R_{17b} is selected from

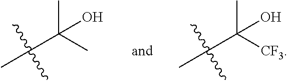

In one embodiment, when R_{17b} is

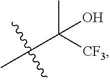 [SMILES: *C(C)(O)C(F)(F)F]

R_{10b} is H; and when R_{17b} is

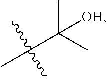 [SMILES: *C(C)(C)O]

t least one of R₁₁, R₁₂, and R₁₃ is present and selected from C₁-C₆ alkoxy and halogen. In one embodiment, the compound of Formula (Ib-5010-WO50) is selected from:

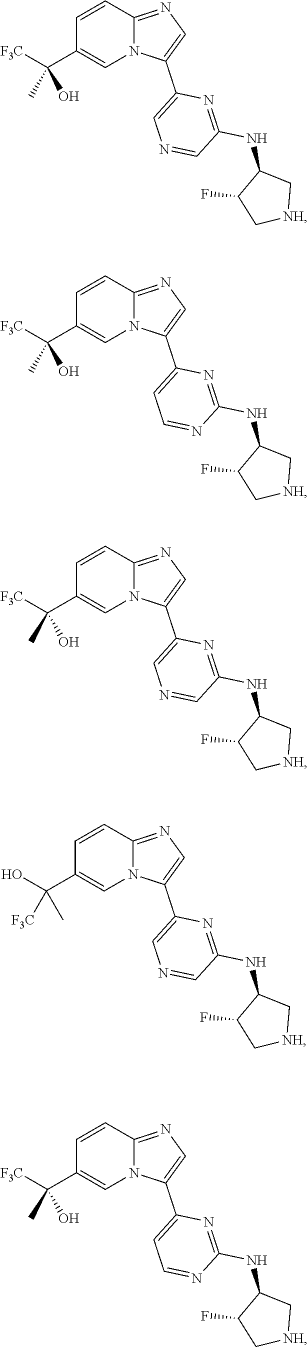 [SMILES: C[C@@](O)(c1ccc2ncc(-c3cncc(N[C@H]4CNC[C@@H]4F)n3)n2c1)C(F)(F)F ; C[C@@](O)(c1ccc2ncc(-c3ccnc(N[C@H]4CNC[C@@H]4F)n3)n2c1)C(F)(F)F ; C[C@](O)(c1ccc2ncc(-c3cncc(N[C@H]4CNC[C@@H]4F)n3)n2c1)C(F)(F)F ; CC(O)(c1ccc2ncc(-c3cncc(N[C@H]4CNC[C@@H]4F)n3)n2c1)C(F)(F)F ; C[C@](O)(c1ccc2ncc(-c3ccnc(N[C@H]4CNC[C@@H]4F)n3)n2c1)C(F)(F)F]

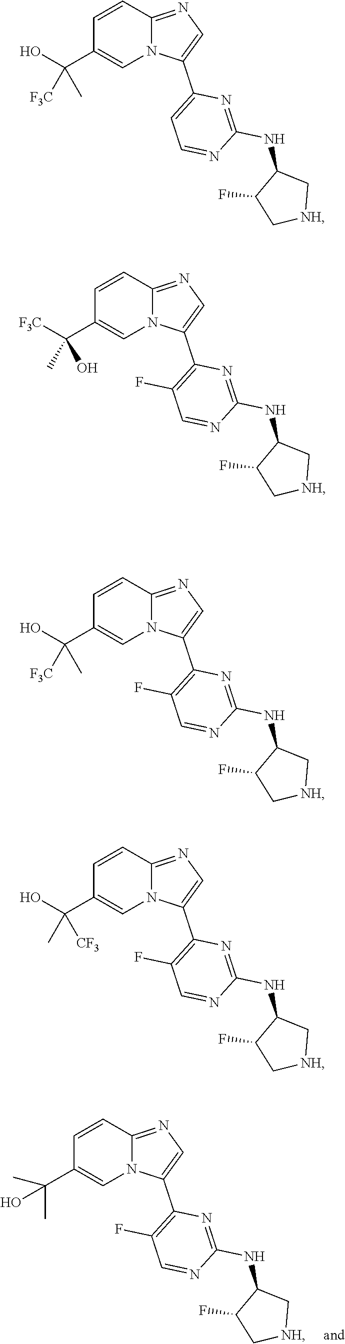 [SMILES: CC(O)(c1ccc2ncc(-c3ccnc(N[C@H]4CNC[C@@H]4F)n3)n2c1)C(F)(F)F ; C[C@@](O)(c1ccc2ncc(-c3nc(N[C@H]4CNC[C@@H]4F)ncc3F)n2c1)C(F)(F)F ; CC(O)(c1ccc2ncc(-c3nc(N[C@H]4CNC[C@@H]4F)ncc3F)n2c1)C(F)(F)F ; CC(O)(c1ccc2ncc(-c3nc(N[C@H]4CNC[C@@H]4F)ncc3F)n2c1)C(F)(F)F ; CC(C)(O)c1ccc2ncc(-c3nc(N[C@H]4CNC[C@@H]4F)ncc3F)n2c1]

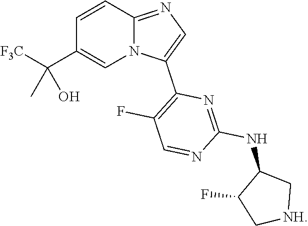 [SMILES: CC(O)(c1ccc2ncc(-c3nc(N[C@H]4CNC[C@@H]4F)ncc3F)n2c1)C(F)(F)F]

In one embodiment, the compound of Formula (I) is a compound of Formula (Id-5010-WO50):

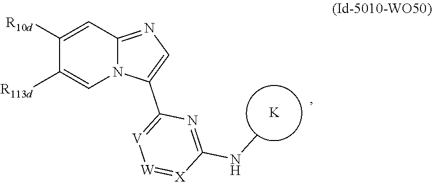

or a salt, ester, solvate, optical isomer, geometric isomer, or salt of an isomer thereof; wherein: V is N or CR₁₁; W is N or CR₁₂; X is N or CR₁₃;

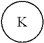

is

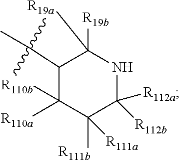

R_{10d} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and halogen, and C₃-C₆ cycloalkyl and wherein —O—(C₃-C₆ cycloalkyl) are each optionally substituted with one or more substituents selected from C₁-C₆ alkyl and halogen; R_{113d} is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from —OH, C₁-C₆ alkyl, and halogen; R₁₁, R₁₂, and R₁₃ are each independently selected from H, C₁-C₆ alkoxy, and halogen; R₁₈ₐ and R_{18b} are each independently selected from H, C₁-C₆ alkyl, and C₃-C₆ cycloalkyl, wherein C₁-C₆ alkyl and C₃-C₆ cycloalkyl are each optionally substituted with one or more halogen; R₁₉ₐ, R_{19b}, R₁₁₀ₐ, R_{110b}, R₁₁₁ₐ, R_{111b}, R₁₁₂ₐ, and R_{112b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and one of V, W, or X is N. In one embodiment, at least one of (i)-(vi) applies: (i) each of R₁₉ₐ, R_{19b}, R₁₁₀ₐ, R_{110b}, R₁₁₁ₐ, R_{111b}, R₁₁₂ₐ, and R_{112b} is H; (ii) each of R₁₉ₐ, R_{19b}, R_{110b}, R₁₁₁ₐ, R_{111b}, R₁₁₂ₐ, and R_{112b} is H and R₁₁₀ₐ, is F; (iii) R₁₁, R₁₂, and R₁₃, if present, are H; (iv) V is CR₁₁ wherein R₁₁ is F, W is CR₁₂ wherein R₁₂ is H, and X is N; (v) R_{10d} is selected from H and —OCH₃; and (vi) R_{113d} is selected from

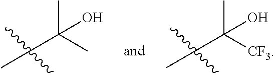

In one embodiment, when R_{113d} is

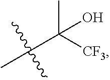 [SMILES: *C(C)(O)C(F)(F)F]

R_{10d} is H; and when R_{113d} is

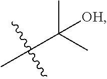 [SMILES: *C(C)(C)O]

at least one of R₁₁, R₁₂, and R₁₃ is present and selected from C₁-C₆ alkoxy and halogen. In one embodiment, the compound of Formula (Id-5010-WO50) is selected from

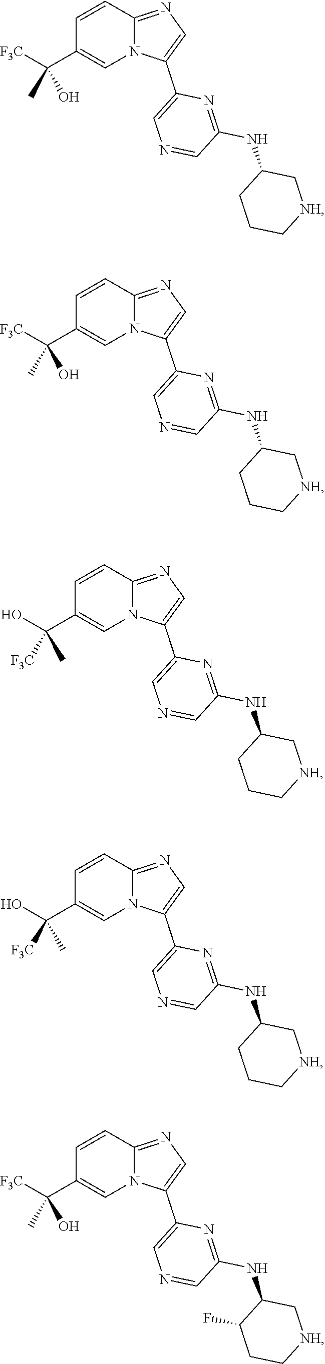 [SMILES: C[C@](O)(c1ccc2ncc(-c3cncc(N[C@H]4CCCNC4)n3)n2c1)C(F)(F)F ; C[C@@](O)(c1ccc2ncc(-c3cncc(N[C@H]4CCCNC4)n3)n2c1)C(F)(F)F ; C[C@@](O)(c1ccc2ncc(-c3cncc(N[C@@H]4CCCNC4)n3)n2c1)C(F)(F)F ; C[C@](O)(c1ccc2ncc(-c3cncc(N[C@@H]4CCCNC4)n3)n2c1)C(F)(F)F ; C[C@@](O)(c1ccc2ncc(-c3cncc(N[C@H]4CNCC[C@@H]4F)n3)n2c1)C(F)(F)F]

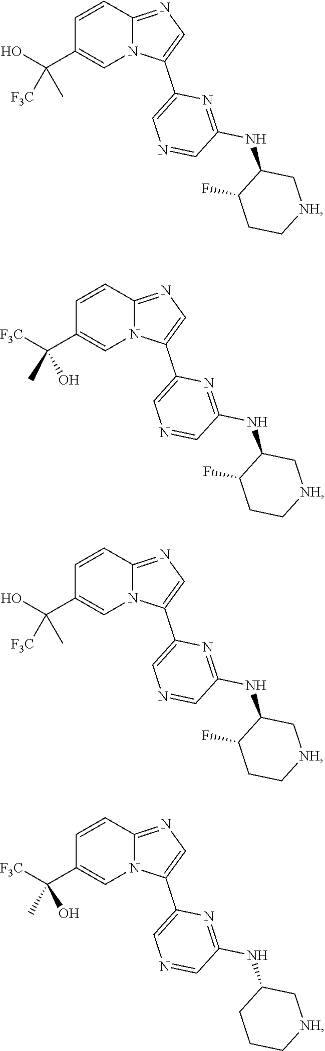 [SMILES: CC(O)(c1ccc2ncc(-c3cncc(N[C@H]4CNCC[C@@H]4F)n3)n2c1)C(F)(F)F ; C[C@](O)(c1ccc2ncc(-c3cncc(N[C@H]4CNCC[C@@H]4F)n3)n2c1)C(F)(F)F ; CC(O)(c1ccc2ncc(-c3cncc(N[C@H]4CNCC[C@@H]4F)n3)n2c1)C(F)(F)F ; C[C@@](O)(c1ccc2ncc(-c3cncc(N[C@H]4CCCNC4)n3)n2c1)C(F)(F)F]

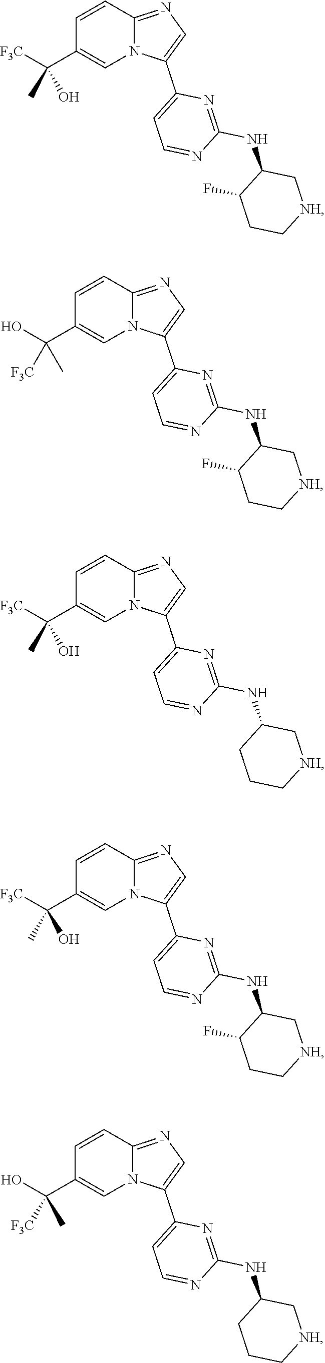 [SMILES: C[C@](O)(c1ccc2ncc(-c3ccnc(N[C@H]4CNCC[C@@H]4F)n3)n2c1)C(F)(F)F ; CC(O)(c1ccc2ncc(-c3ccnc(N[C@H]4CNCC[C@@H]4F)n3)n2c1)C(F)(F)F ; C[C@](O)(c1ccc2ncc(-c3ccnc(N[C@H]4CCCNC4)n3)n2c1)C(F)(F)F ; C[C@@](O)(c1ccc2ncc(-c3ccnc(N[C@H]4CNCC[C@@H]4F)n3)n2c1)C(F)(F)F ; C[C@@](O)(c1ccc2ncc(-c3ccnc(N[C@@H]4CCCNC4)n3)n2c1)C(F)(F)F]

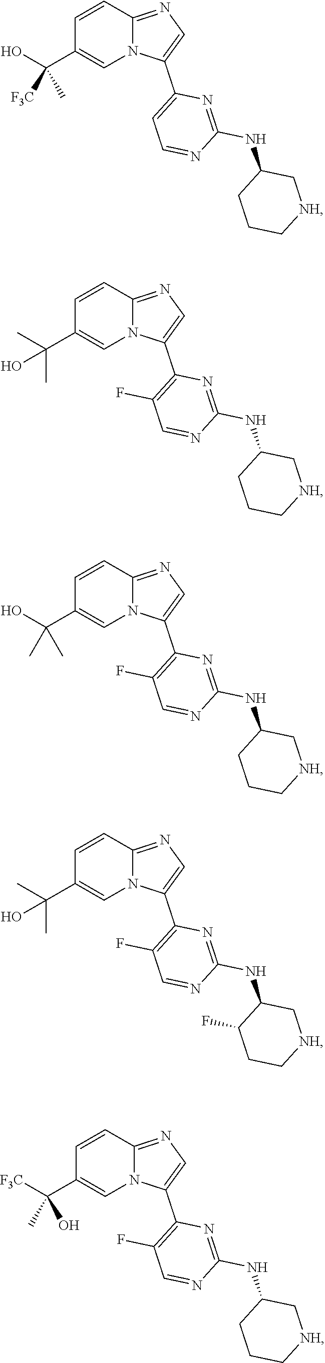 [SMILES: C[C@](O)(c1ccc2ncc(-c3ccnc(N[C@@H]4CCCNC4)n3)n2c1)C(F)(F)F ; CC(C)(O)c1ccc2ncc(-c3nc(N[C@H]4CCCNC4)ncc3F)n2c1 ; CC(C)(O)c1ccc2ncc(-c3nc(N[C@@H]4CCCNC4)ncc3F)n2c1 ; CC(C)(O)c1ccc2ncc(-c3nc(N[C@H]4CNCC[C@@H]4F)ncc3F)n2c1 ; C[C@@](O)(c1ccc2ncc(-c3nc(N[C@H]4CCCNC4)ncc3F)n2c1)C(F)(F)F]

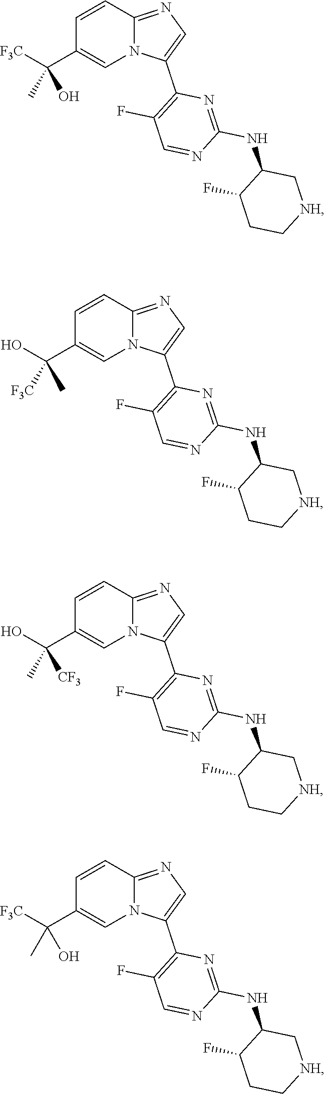 [SMILES: C[C@@](O)(c1ccc2ncc(-c3nc(N[C@H]4CNCC[C@@H]4F)ncc3F)n2c1)C(F)(F)F ; C[C@@](O)(c1ccc2ncc(-c3nc(N[C@H]4CNCC[C@@H]4F)ncc3F)n2c1)C(F)(F)F ; C[C@](O)(c1ccc2ncc(-c3nc(N[C@H]4CNCC[C@@H]4F)ncc3F)n2c1)C(F)(F)F ; CC(O)(c1ccc2ncc(-c3nc(N[C@H]4CNCC[C@@H]4F)ncc3F)n2c1)C(F)(F)F]

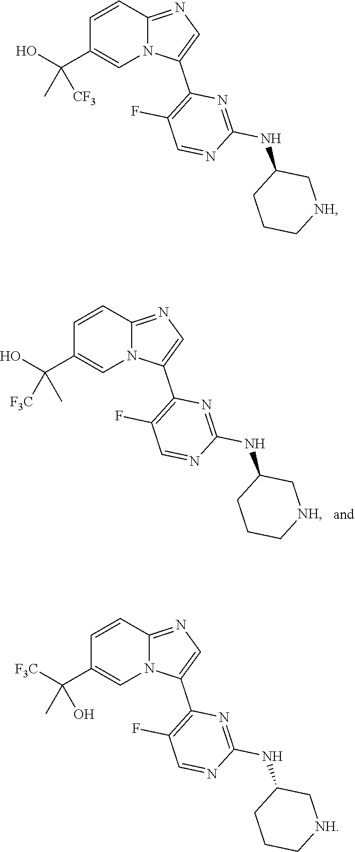 [SMILES: CC(O)(c1ccc2ncc(-c3nc(N[C@@H]4CCCNC4)ncc3F)n2c1)C(F)(F)F ; CC(O)(c1ccc2ncc(-c3nc(N[C@@H]4CCCNC4)ncc3F)n2c1)C(F)(F)F ; CC(O)(c1ccc2ncc(-c3nc(N[C@H]4CCCNC4)ncc3F)n2c1)C(F)(F)F]

In one embodiment the compound of Formula (II) is a compound of Formula (IIa-5010-WO50):

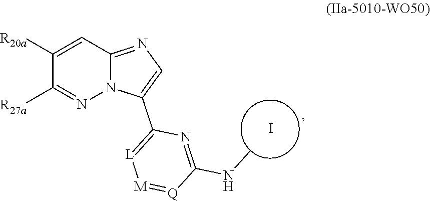

or a salt, ester, solvate, optical isomer, geometric isomer, or salt of an isomer thereof; wherein: L is N or CR₂₁; M is N or CR₂₂; Q is N or CR₂₃;

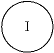

is

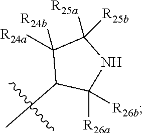

R₂₀ₐ is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from C₁-C₆ alkyl and halogen; R₂₇ₐ is selected from halogen, C₁-C₆ alkyl, C₁-C₆ alkoxy, C₃-C₆ cycloalkyl, spiro-fused cycloalkyl, —O—(C₃-C₆ cycloalkyl), C₂-C₆ heterocyclyl, imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl, spiro-cycloalkyl, —O—(C₃-C₆ cycloalkyl), and C₂-C₆ heterocyclyl are each optionally substituted with one or more substituents selected from —OH, C₁-C₆ alkyl, and halogen; R₂₁, R₂₂, and R₂₃ are each independently selected from H, C₁-C₆ alkoxy, and halogen; R₂₄ₐ, R_{24b}, R₂₅ₐ, R_{25b}, R₂₆ₐ, and R_{26b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R₂₈ₐ and R_{28b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of L, M, or Q is N. In one embodiment, at least one of (i)-(v) applies: (i) each of R_{24b}, R₂₅ₐ, R_{25b}, R₂₆ₐ, and R_{26b} is H and R₂₄ₐ is F; (ii) R₂₁, R₂₂, and R₂₃, if present, are H; (iii) L is CR₂₁ wherein R₂₁ is selected from F, Cl, and —OCH₃, M is CR₂₂ wherein R₂₂ is H, and Q is N; (iv) R₂₀ₐ is selected from —OCH₃, unsubstituted —O—(C₃ cycloalkyl), and

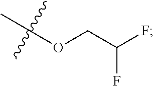 [SMILES: *OCC(F)F]

and (v) R₂₇ₐ is selected from unsubstituted C₃-C₆ cycloalkyl,

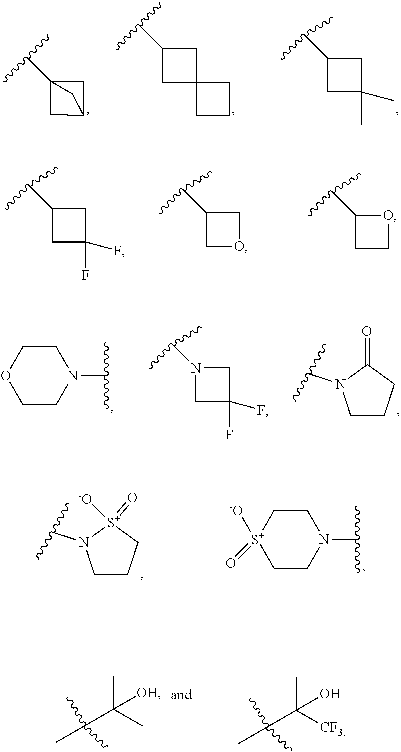

In one embodiment, when R₂₀ₐ is —OCH₃ and R₂₇ₐ is unsubstituted C₃ cycloalkyl, at least one of R₂₁, R₂₂, and R₂₃ is present and selected from C₁-C₆ alkoxy and halogen. In one embodiment, the compound of Formula (IIa-5010-WO50) is selected from:

[chemical structure]

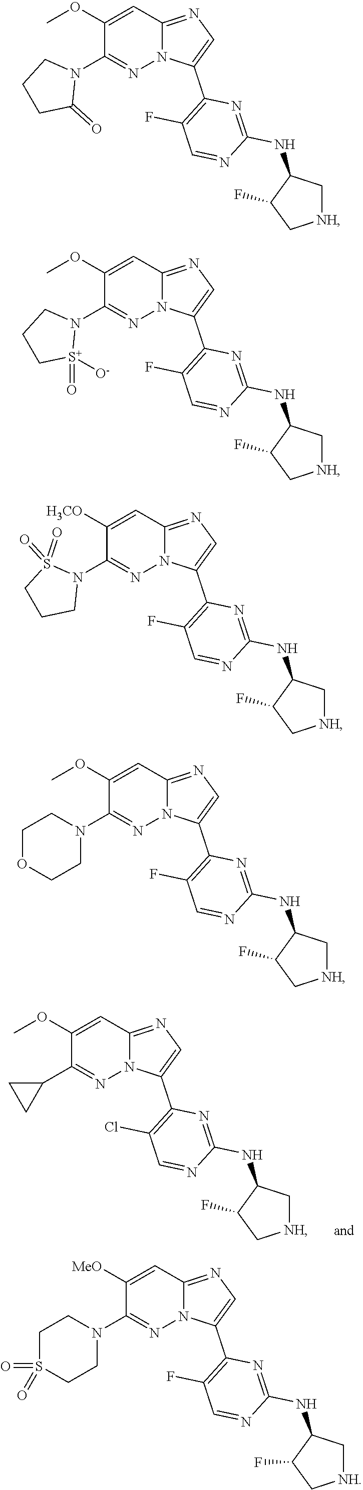

In one embodiment, the compound of Formula (II) is a compound of Formula (IIb-5010-WO50):

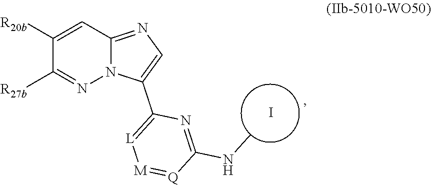

or a salt, ester, solvate, optical isomer, geometric isomer, or salt of an isomer thereof; wherein: L is N or CR₂₁; M is N or CR₂₂; Q is N or CR₂₃;

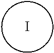

is

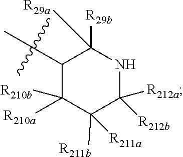

R_{20b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from C₁-C₆ alkyl and halogen; R_{27b} is selected from halogen, C₁-C₆ alkyl, C₁-C₆ alkoxy, C₃-C₆ cycloalkyl, spiro-cycloalkyl, —O—(C₃-C₆ cycloalkyl), C₂-C₆ heterocyclyl, imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl, spiro-cycloalkyl, —O—(C₃-C₆ cycloalkyl), and C₂-C₆ heterocyclyl are each optionally substituted with one or more substituents selected from —OH, C₁-C₆ alkyl, and halogen; R₂₁, R₂₂, and R₂₃ are each independently selected from H, C₁-C₆ alkoxy, and halogen; R₂₉ₐ, R_{29b}, R₂₁₀ₐ, R_{210b}, R₂₁₁ₐ, R_{211b}, R₂₁₂ₐ, and R_{212b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R₂₈ₐ and R_{28b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of L, M, or Q is N. In one embodiment, at least one of (i)-(vi) applies: (i) each of R₂₉ₐ, R_{29b}, R₂₁₀ₐ, R_{210b}, R₂₁₁ₐ, R_{211b}, R₂₁₂ₐ, and R_{212b} is H; (ii) each of R₂₉ₐ, R_{29b}, R_{210b}, R₂₁₁ₐ, R_{211b}, R₂₁₂ₐ, and R_{212b} is H and R₂₁₀ₐ is F; (iii) R₂₁, R₂₂, and R₂₃, if present, are H; (iv) L is CR₂₁ wherein R₂₁ is selected from F, Cl, and —OCH₃, M is CR₂₂ wherein R₂₂ is H, and Q is N; (v) R_{20b} is selected from —OCH₃, —OCD₃, unsubstituted —O—(C₃ cycloalkyl), and

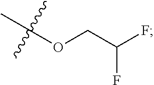 [SMILES: *OCC(F)F]

and (vi) R_{27b} is selected from unsubstituted C₃-C₆ cycloalkyl,

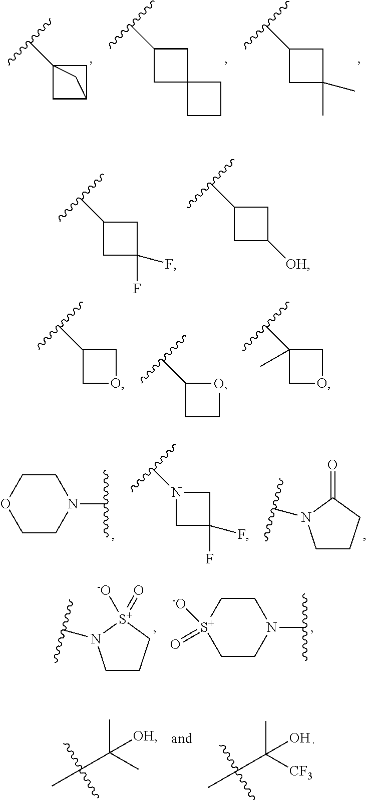

In one embodiment, when R_{20b} is —OCH₃ and R_{27b} is unsubstituted C₃ cycloalkyl, at least one of R₂₁, R₂₂, and R₂₃ is present and selected from C₁-C₆ alkoxy and halogen. In one embodiment, the compound of Formula (IIb-5010-WO50) is selected from:

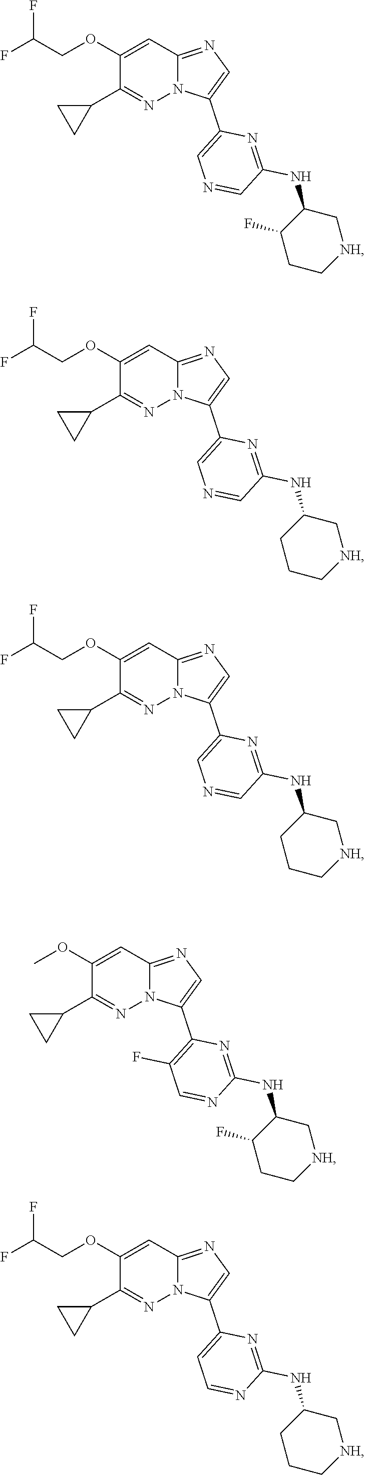 [SMILES: FC(F)COc1cc2ncc(-c3cncc(N[C@H]4CNCC[C@@H]4F)n3)n2nc1C1CC1 ; FC(F)COc1cc2ncc(-c3cncc(N[C@H]4CCCNC4)n3)n2nc1C1CC1 ; FC(F)COc1cc2ncc(-c3cncc(N[C@@H]4CCCNC4)n3)n2nc1C1CC1 ; COc1cc2ncc(-c3nc(N[C@H]4CNCC[C@@H]4F)ncc3F)n2nc1C1CC1 ; FC(F)COc1cc2ncc(-c3ccnc(N[C@H]4CCCNC4)n3)n2nc1C1CC1]

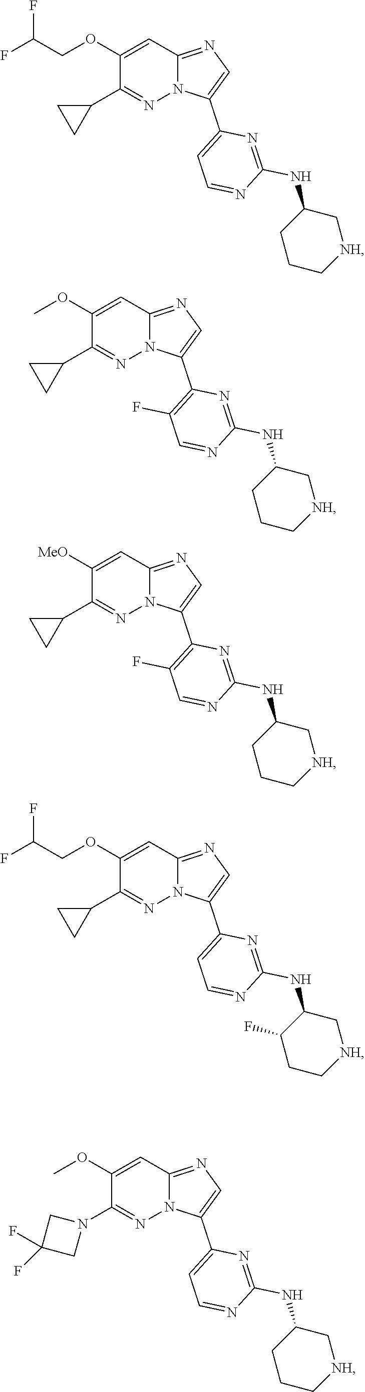 [SMILES: FC(F)COc1cc2ncc(-c3ccnc(N[C@@H]4CCCNC4)n3)n2nc1C1CC1 ; COc1cc2ncc(-c3nc(N[C@H]4CCCNC4)ncc3F)n2nc1C1CC1 ; COc1cc2ncc(-c3nc(N[C@@H]4CCCNC4)ncc3F)n2nc1C1CC1 ; FC(F)COc1cc2ncc(-c3ccnc(N[C@H]4CNCC[C@@H]4F)n3)n2nc1C1CC1 ; COc1cc2ncc(-c3ccnc(N[C@H]4CCCNC4)n3)n2nc1N1CC(F)(F)C1]

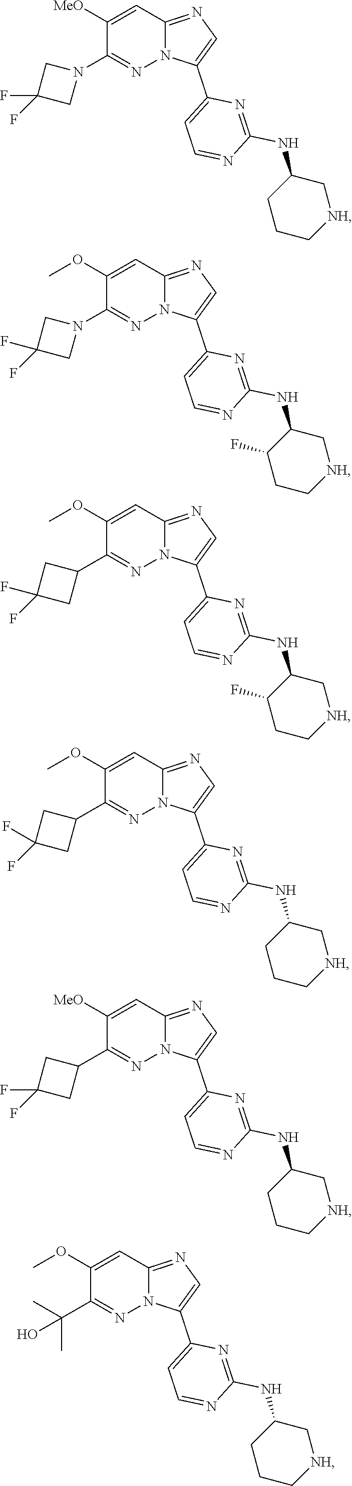 [SMILES: COc1cc2ncc(-c3ccnc(N[C@@H]4CCCNC4)n3)n2nc1N1CC(F)(F)C1 ; COc1cc2ncc(-c3ccnc(N[C@H]4CNCC[C@@H]4F)n3)n2nc1N1CC(F)(F)C1 ; COc1cc2ncc(-c3ccnc(N[C@H]4CNCC[C@@H]4F)n3)n2nc1C1CC(F)(F)C1 ; COc1cc2ncc(-c3ccnc(N[C@H]4CCCNC4)n3)n2nc1C1CC(F)(F)C1 ; COc1cc2ncc(-c3ccnc(N[C@@H]4CCCNC4)n3)n2nc1C1CC(F)(F)C1 ; COc1cc2ncc(-c3ccnc(N[C@H]4CCCNC4)n3)n2nc1C(C)(C)O]

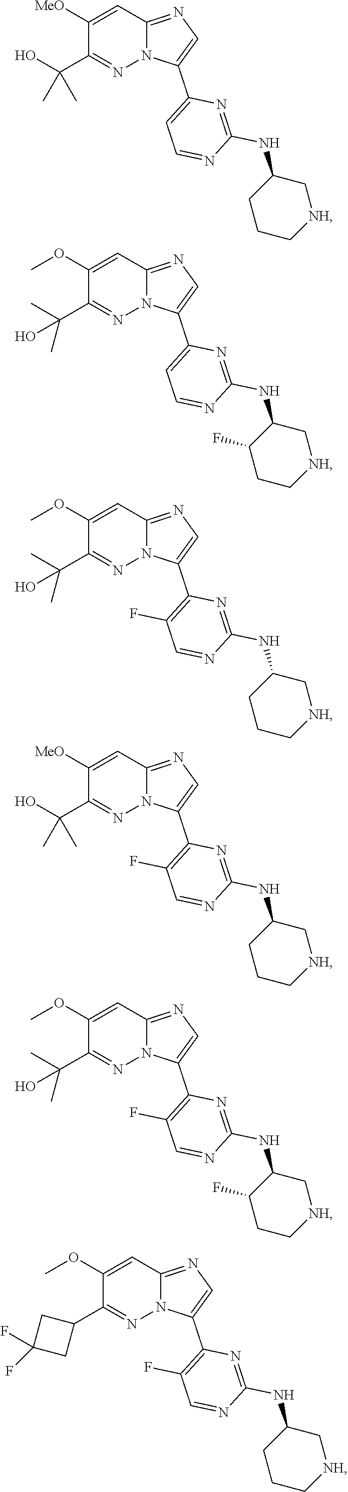 [SMILES: COc1cc2ncc(-c3ccnc(N[C@@H]4CCCNC4)n3)n2nc1C(C)(C)O ; COc1cc2ncc(-c3ccnc(N[C@H]4CNCC[C@@H]4F)n3)n2nc1C(C)(C)O ; COc1cc2ncc(-c3nc(N[C@H]4CCCNC4)ncc3F)n2nc1C(C)(C)O ; COc1cc2ncc(-c3nc(N[C@@H]4CCCNC4)ncc3F)n2nc1C(C)(C)O ; COc1cc2ncc(-c3nc(N[C@H]4CNCC[C@@H]4F)ncc3F)n2nc1C(C)(C)O ; COc1cc2ncc(-c3nc(N[C@@H]4CCCNC4)ncc3F)n2nc1C1CC(F)(F)C1]

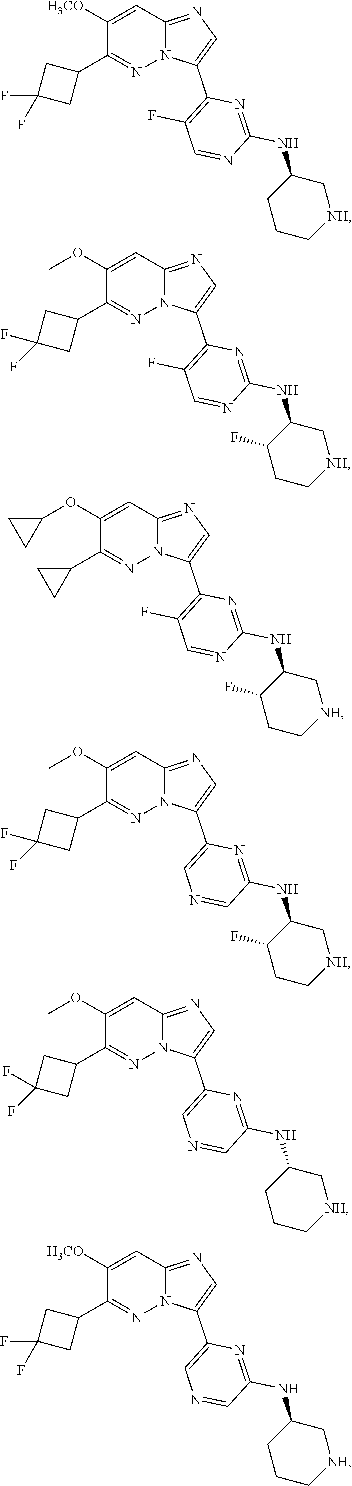 [SMILES: COc1cc2ncc(-c3nc(N[C@@H]4CCCNC4)ncc3F)n2nc1C1CC(F)(F)C1 ; COc1cc2ncc(-c3nc(N[C@H]4CNCC[C@@H]4F)ncc3F)n2nc1C1CC(F)(F)C1 ; Fc1cnc(N[C@H]2CNCC[C@@H]2F)nc1-c1cnc2cc(OC3CC3)c(C3CC3)nn12 ; COc1cc2ncc(-c3cncc(N[C@H]4CNCC[C@@H]4F)n3)n2nc1C1CC(F)(F)C1 ; COc1cc2ncc(-c3cncc(N[C@H]4CCCNC4)n3)n2nc1C1CC(F)(F)C1 ; COc1cc2ncc(-c3cncc(N[C@@H]4CCCNC4)n3)n2nc1C1CC(F)(F)C1]

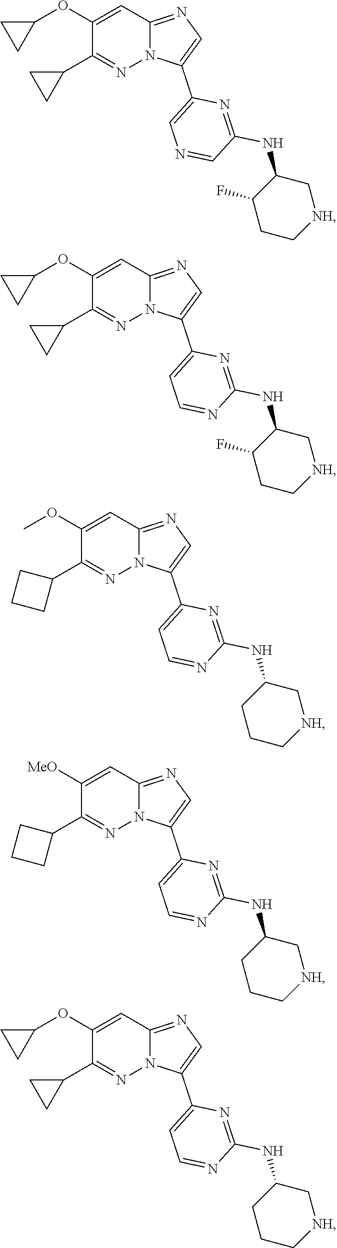 [SMILES: F[C@H]1CCNC[C@@H]1Nc1cncc(-c2cnc3cc(OC4CC4)c(C4CC4)nn23)n1 ; F[C@H]1CCNC[C@@H]1Nc1nccc(-c2cnc3cc(OC4CC4)c(C4CC4)nn23)n1 ; COc1cc2ncc(-c3ccnc(N[C@H]4CCCNC4)n3)n2nc1C1CCC1 ; COc1cc2ncc(-c3ccnc(N[C@@H]4CCCNC4)n3)n2nc1C1CCC1 ; c1cc(-c2cnc3cc(OC4CC4)c(C4CC4)nn23)nc(N[C@H]2CCCNC2)n1]

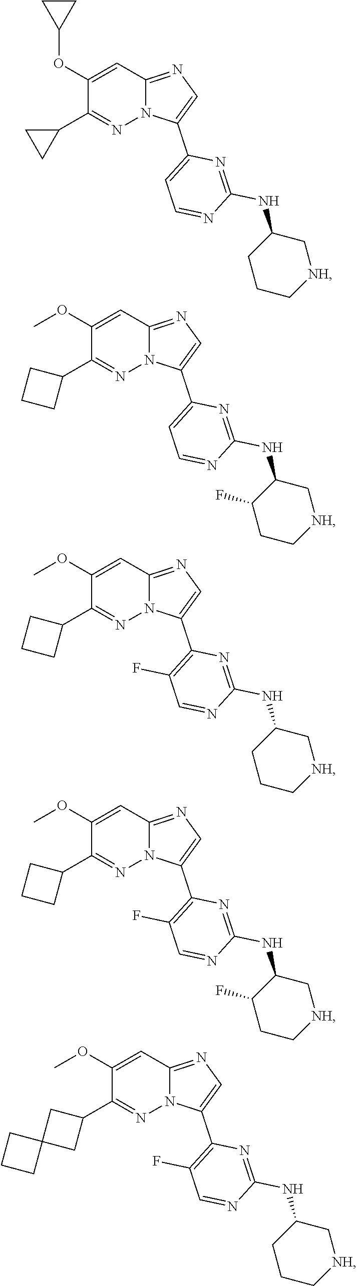 [SMILES: c1cc(-c2cnc3cc(OC4CC4)c(C4CC4)nn23)nc(N[C@@H]2CCCNC2)n1 ; COc1cc2ncc(-c3ccnc(N[C@H]4CNCC[C@@H]4F)n3)n2nc1C1CCC1 ; COc1cc2ncc(-c3nc(N[C@H]4CCCNC4)ncc3F)n2nc1C1CCC1 ; COc1cc2ncc(-c3nc(N[C@H]4CNCC[C@@H]4F)ncc3F)n2nc1C1CCC1 ; COc1cc2ncc(-c3nc(N[C@H]4CCCNC4)ncc3F)n2nc1C1CC2(CCC2)C1]

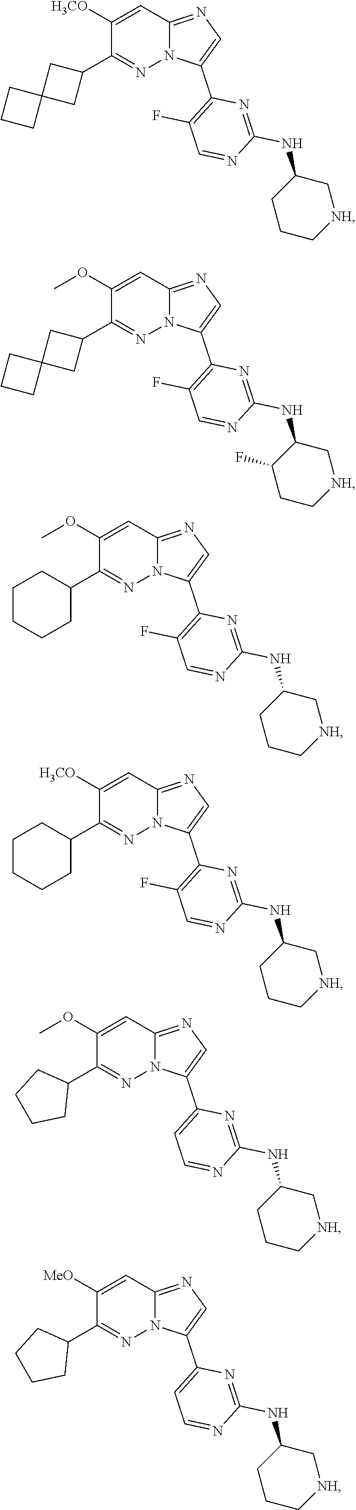 [SMILES: COc1cc2ncc(-c3nc(N[C@@H]4CCCNC4)ncc3F)n2nc1C1CC2(CCC2)C1 ; COc1cc2ncc(-c3nc(N[C@H]4CNCC[C@@H]4F)ncc3F)n2nc1C1CC2(CCC2)C1 ; COc1cc2ncc(-c3nc(N[C@H]4CCCNC4)ncc3F)n2nc1C1CCCCC1 ; COc1cc2ncc(-c3nc(N[C@@H]4CCCNC4)ncc3F)n2nc1C1CCCCC1 ; COc1cc2ncc(-c3ccnc(N[C@H]4CCCNC4)n3)n2nc1C1CCCC1 ; COc1cc2ncc(-c3ccnc(N[C@@H]4CCCNC4)n3)n2nc1C1CCCC1]

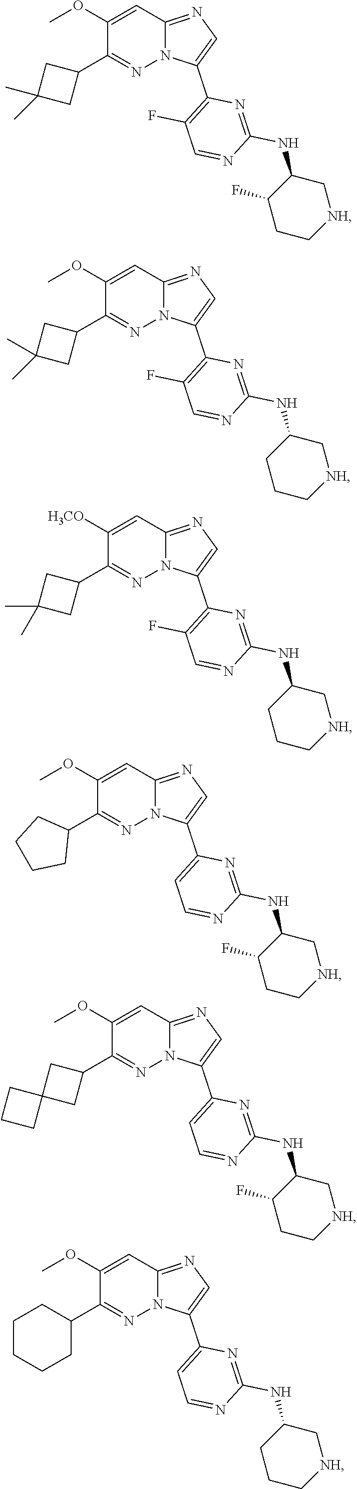 [SMILES: COc1cc2ncc(-c3nc(N[C@H]4CNCC[C@@H]4F)ncc3F)n2nc1C1CC(C)(C)C1 ; COc1cc2ncc(-c3nc(N[C@H]4CCCNC4)ncc3F)n2nc1C1CC(C)(C)C1 ; COc1cc2ncc(-c3nc(N[C@@H]4CCCNC4)ncc3F)n2nc1C1CC(C)(C)C1 ; COc1cc2ncc(-c3ccnc(N[C@H]4CNCC[C@@H]4F)n3)n2nc1C1CCCC1 ; COc1cc2ncc(-c3ccnc(N[C@H]4CNCC[C@@H]4F)n3)n2nc1C1CC2(CCC2)C1 ; COc1cc2ncc(-c3ccnc(N[C@H]4CCCNC4)n3)n2nc1C1CCCCC1]

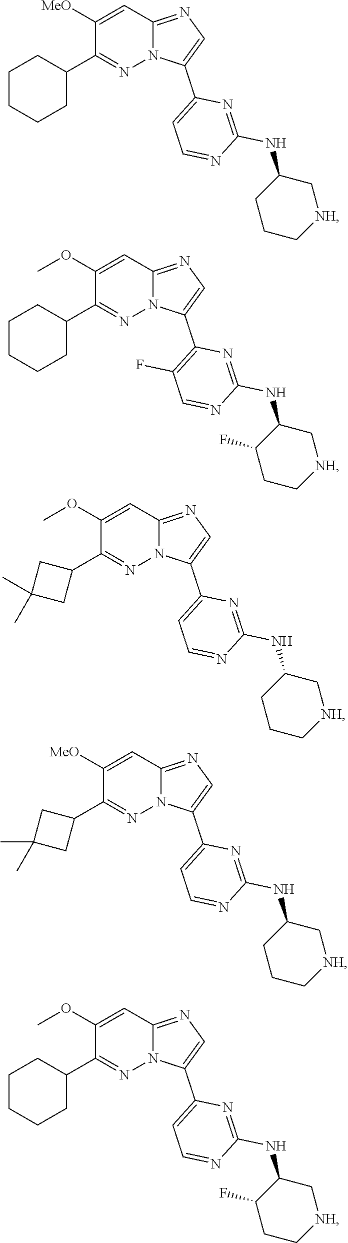 [SMILES: COc1cc2ncc(-c3ccnc(N[C@@H]4CCCNC4)n3)n2nc1C1CCCCC1 ; COc1cc2ncc(-c3nc(N[C@H]4CNCC[C@@H]4F)ncc3F)n2nc1C1CCCCC1 ; COc1cc2ncc(-c3ccnc(N[C@H]4CCCNC4)n3)n2nc1C1CC(C)(C)C1 ; COc1cc2ncc(-c3ccnc(N[C@@H]4CCCNC4)n3)n2nc1C1CC(C)(C)C1 ; COc1cc2ncc(-c3ccnc(N[C@H]4CNCC[C@@H]4F)n3)n2nc1C1CCCCC1]

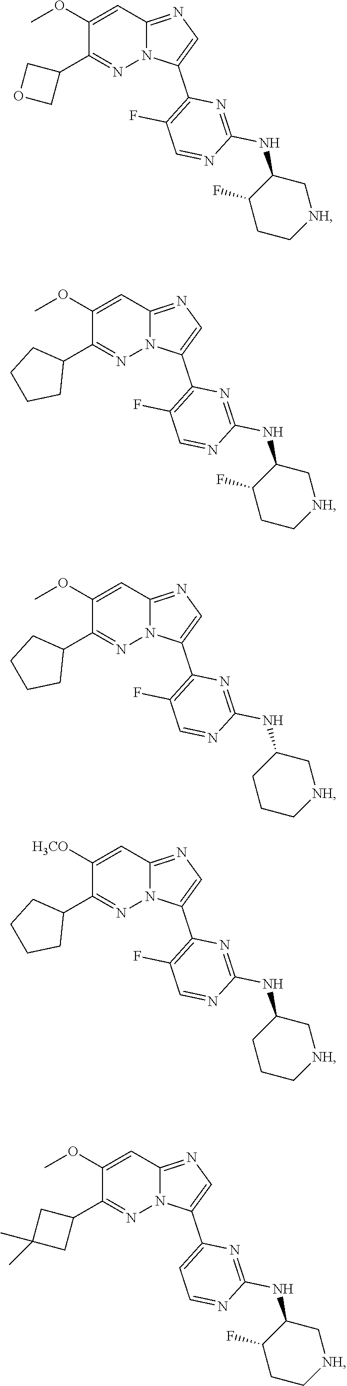 [SMILES: COc1cc2ncc(-c3nc(N[C@H]4CNCC[C@@H]4F)ncc3F)n2nc1C1COC1 ; COc1cc2ncc(-c3nc(N[C@H]4CNCC[C@@H]4F)ncc3F)n2nc1C1CCCC1 ; COc1cc2ncc(-c3nc(N[C@H]4CCCNC4)ncc3F)n2nc1C1CCCC1 ; COc1cc2ncc(-c3nc(N[C@@H]4CCCNC4)ncc3F)n2nc1C1CCCC1 ; COc1cc2ncc(-c3ccnc(N[C@H]4CNCC[C@@H]4F)n3)n2nc1C1CC(C)(C)C1]

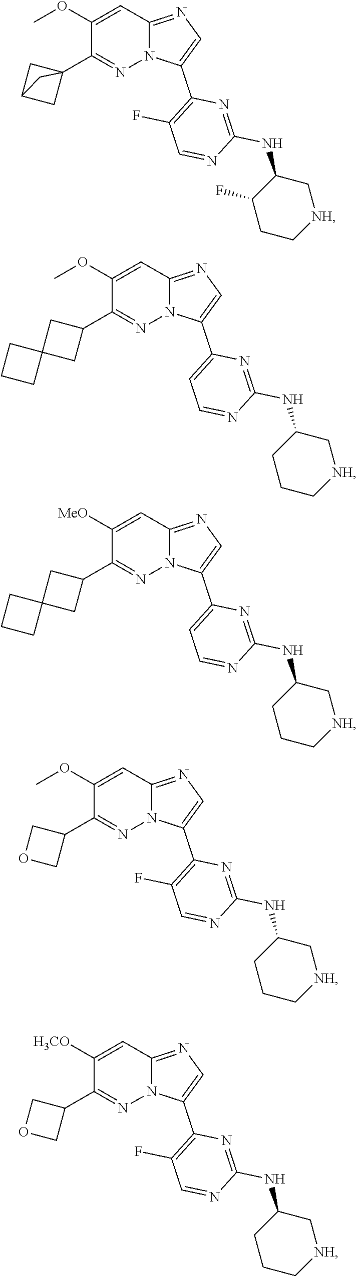 [SMILES: COc1cc2ncc(-c3nc(N[C@H]4CNCC[C@@H]4F)ncc3F)n2nc1C12CC(C1)C2 ; COc1cc2ncc(-c3ccnc(N[C@H]4CCCNC4)n3)n2nc1C1CC2(CCC2)C1 ; COc1cc2ncc(-c3ccnc(N[C@@H]4CCCNC4)n3)n2nc1C1CC2(CCC2)C1 ; COc1cc2ncc(-c3nc(N[C@H]4CCCNC4)ncc3F)n2nc1C1COC1 ; COc1cc2ncc(-c3nc(N[C@@H]4CCCNC4)ncc3F)n2nc1C1COC1]

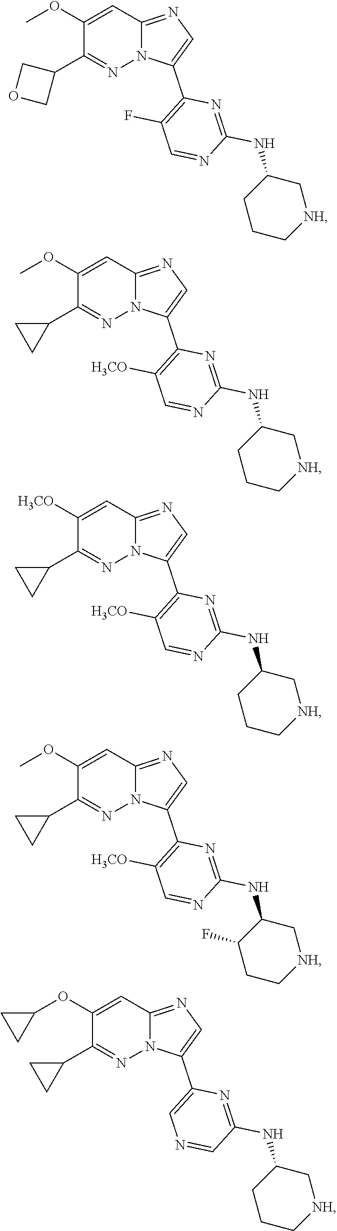 [SMILES: COc1cc2ncc(-c3nc(N[C@H]4CCCNC4)ncc3F)n2nc1C1COC1 ; COc1cnc(N[C@H]2CCCNC2)nc1-c1cnc2cc(OC)c(C3CC3)nn12 ; COc1cnc(N[C@@H]2CCCNC2)nc1-c1cnc2cc(OC)c(C3CC3)nn12 ; COc1cnc(N[C@H]2CNCC[C@@H]2F)nc1-c1cnc2cc(OC)c(C3CC3)nn12 ; c1ncc(-c2cnc3cc(OC4CC4)c(C4CC4)nn23)nc1N[C@H]1CCCNC1]

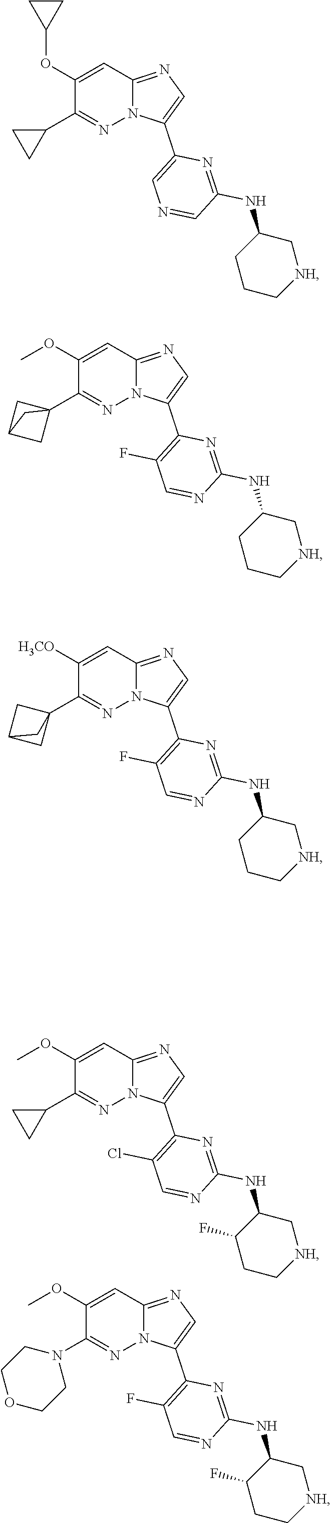 [SMILES: c1ncc(-c2cnc3cc(OC4CC4)c(C4CC4)nn23)nc1N[C@@H]1CCCNC1 ; COc1cc2ncc(-c3nc(N[C@H]4CCCNC4)ncc3F)n2nc1C12CC(C1)C2 ; COc1cc2ncc(-c3nc(N[C@@H]4CCCNC4)ncc3F)n2nc1C12CC(C1)C2 ; COc1cc2ncc(-c3nc(N[C@H]4CNCC[C@@H]4F)ncc3Cl)n2nc1C1CC1 ; COc1cc2ncc(-c3nc(N[C@H]4CNCC[C@@H]4F)ncc3F)n2nc1N1CCOCC1]

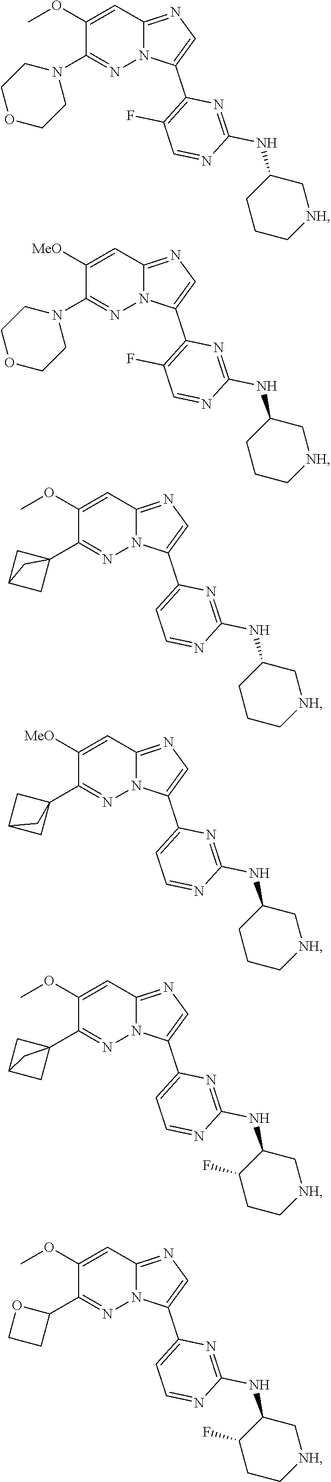 [SMILES: COc1cc2ncc(-c3nc(N[C@H]4CCCNC4)ncc3F)n2nc1N1CCOCC1 ; COc1cc2ncc(-c3nc(N[C@@H]4CCCNC4)ncc3F)n2nc1N1CCOCC1 ; COc1cc2ncc(-c3ccnc(N[C@H]4CCCNC4)n3)n2nc1C12CC(C1)C2 ; COc1cc2ncc(-c3ccnc(N[C@@H]4CCCNC4)n3)n2nc1C12CC(C1)C2 ; COc1cc2ncc(-c3ccnc(N[C@H]4CNCC[C@@H]4F)n3)n2nc1C12CC(C1)C2 ; COc1cc2ncc(-c3ccnc(N[C@H]4CNCC[C@@H]4F)n3)n2nc1C1CCO1]

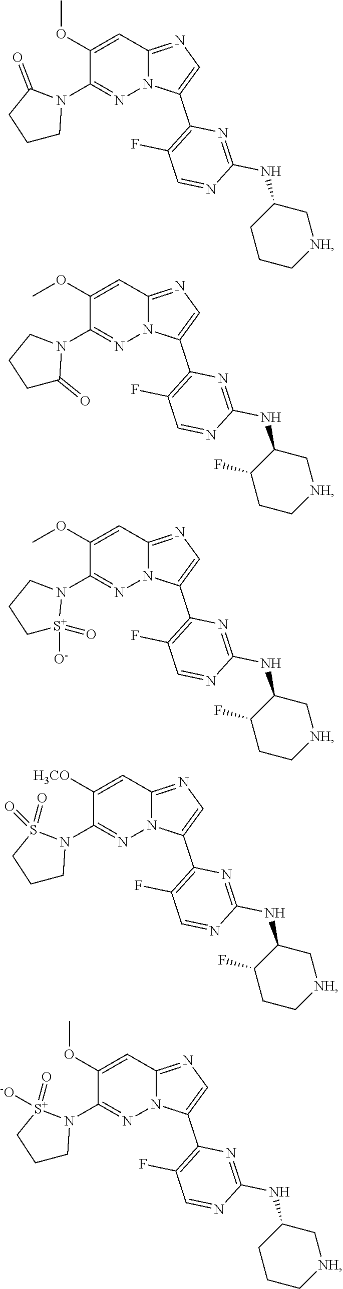

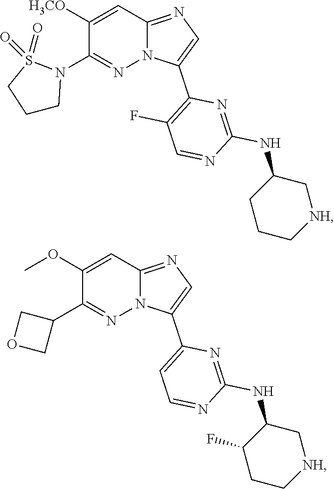 [SMILES: COc1cc2ncc(-c3nc(N[C@@H]4CCCNC4)ncc3F)n2nc1N1CCCS1(=O)=O ; COc1cc2ncc(-c3ccnc(N[C@H]4CNCC[C@@H]4F)n3)n2nc1C1COC1]

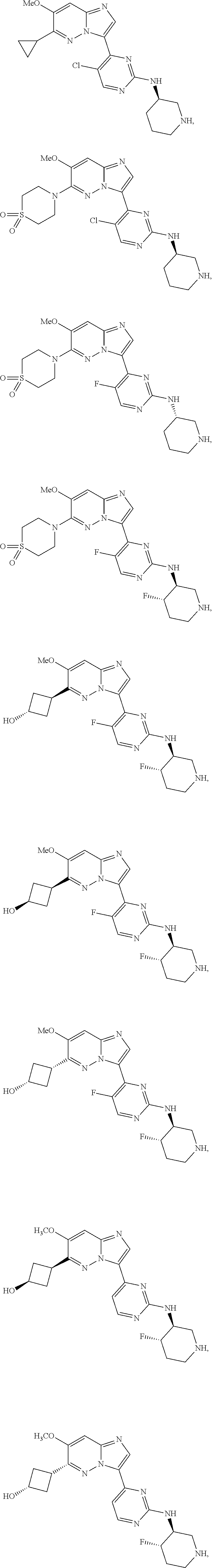

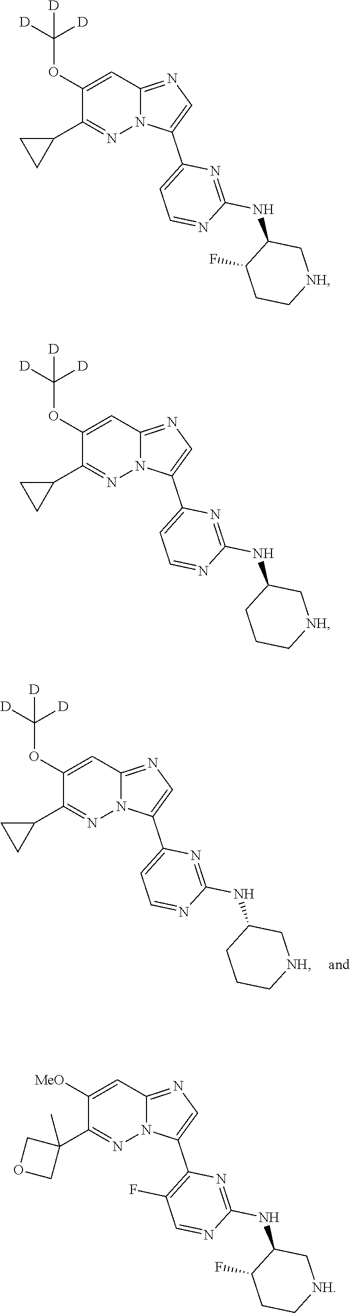 [SMILES: [2H]C([2H])([2H])Oc1cc2ncc(-c3ccnc(N[C@H]4CNCC[C@@H]4F)n3)n2nc1C1CC1 ; [2H]C([2H])([2H])Oc1cc2ncc(-c3ccnc(N[C@@H]4CCCNC4)n3)n2nc1C1CC1 ; [2H]C([2H])([2H])Oc1cc2ncc(-c3ccnc(N[C@H]4CCCNC4)n3)n2nc1C1CC1 ; "and" ; COc1cc2ncc(-c3nc(N[C@H]4CNCC[C@@H]4F)ncc3F)n2nc1C1(C)COC1]

In one embodiment, the compound of Formula (III) is a compound of Formula (IIIa-5010-WO50):

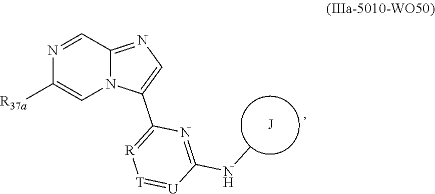

or a salt, ester, solvate, optical isomer, geometric isomer, or salt of an isomer thereof; wherein: R is N or CR₃₁; T is N or CR₃₂; U is N or CR₃₃;

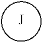

is

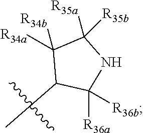

R₃₇ₐ is selected from halogen, C₁-C₆ alkyl, C₁-C₆ alkoxy, C₃-C₆ cycloalkyl, —O—(C₃-C₆ cycloalkyl), C₂-C₆ heterocyclyl, imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH, deuterium, and halogen, and wherein C₃-C₆ cycloalkyl, —O—(C₃-C₆ cycloalkyl), and C₂-C₆ heterocyclyl are each optionally substituted with one or more substituents selected from —OH, C₁-C₆ alkyl, and halogen; R₃₁, R₃₂, and R₃₃ are each independently selected from H, C₁-C₆ alkoxy, and halogen; R₃₄ₐ, R_{34b}, R₃₅ₐ, R_{35b}, R₃₆ₐ, and R_{36b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R₃₈ₐ and R_{38b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of R, T, or U is N. In one embodiment, at least one of (i)-(iv) applies: (i) each of R_{34b}, R₃₅ₐ, R_{35b}, R₃₆ₐ, and R_{36b} is H and R₃₄ₐ is F; (ii) R₃₁, R₃₂, and R₃₃, if present, are H; (iii) R is CR₃₁ wherein R₃₁ is F, T is CR₃₂ wherein R₃₂ is H, and U is N; and (iv) R₃₇ₐ is selected from

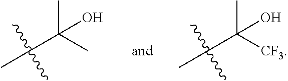

In one embodiment, R₃₇ₐ is not

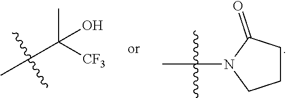

In one embodiment, the compound of Formula (IIIa-5010-WO50) is:

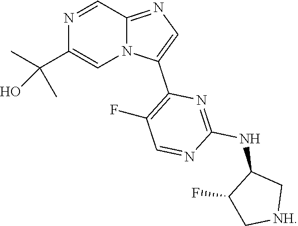 [SMILES: CC(C)(O)c1cn2c(-c3nc(N[C@H]4CNC[C@@H]4F)ncc3F)cnc2cn1]

In one embodiment, the compound of Formula (III) is a compound of Formula (IIIb-5010-WO50):

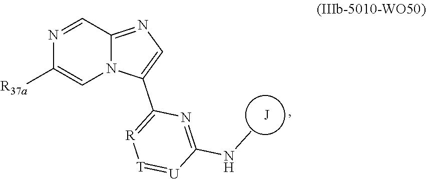

or a salt, ester, solvate, optical isomer, geometric isomer, or salt of an isomer thereof; wherein: R is N or CR₃₁; T is N or CR₃₂; U is N or CR₃₃;

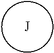

is

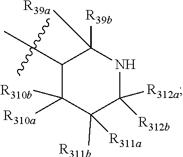

R_{37b} is selected from halogen, C₁-C₆ alkyl, C₁-C₆ alkoxy, C₃-C₆ cycloalkyl, —O—(C₃-C₆ cycloalkyl), C₂-C₆ heterocyclyl, imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH, deuterium, and halogen, and wherein C₃-C₆ cycloalkyl, —O—(C₃-C₆cycloalkyl), and C₂-C₆ heterocyclyl are each optionally substituted with one or more substituents selected from —OH, C₁-C₆ alkyl, and halogen; R₃₁, R₃₂, and R₃₃ are each independently selected from H, C₁-C₆ alkoxy, and halogen; R₃₉ₐ, R_{39b}, R₃₁₀ₐ, R_{310b}, R₃₁₁ₐ, R_{311b}, R₃₁₂ₐ, and R_{312b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R₃₈ₐ and R_{38b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of R, T, or U is N. In one embodiment, at least one of (i)-(v) applies: (i) each of R₃₉ₐ, R_{39b}, R₃₁₀ₐ, R_{310b}, R₃₁₁ₐ, R_{311b}, R₃₁₂ₐ, and R_{312b} is H; (ii) each of R₃₉ₐ, R_{39b}, R_{310b}, R₃₁₁ₐ, R_{311b}, R₃₁₂ₐ, and R_{312b} is H and R₃₁₀ₐ is F; (iii) R₃₁, R₃₂, and R₃₃, if present, are H; (iv) R is CR₃₁ wherein R₃₁ is F, T is CR₃₂ wherein R₃₂ is H, and U is N; and (v) R_{37b} is selected from

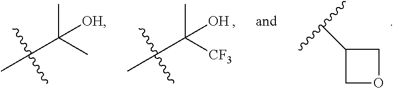

In one embodiment, when R_{37b} is

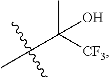 [SMILES: *C(C)(O)C(F)(F)F]

at least one of R₃₁, R₃₂, and R₃₃ is present and selected from C₁-C₆ alkoxy and halogen. In one embodiment, the compound of Formula (IIIb-5010-WO50) is selected from:

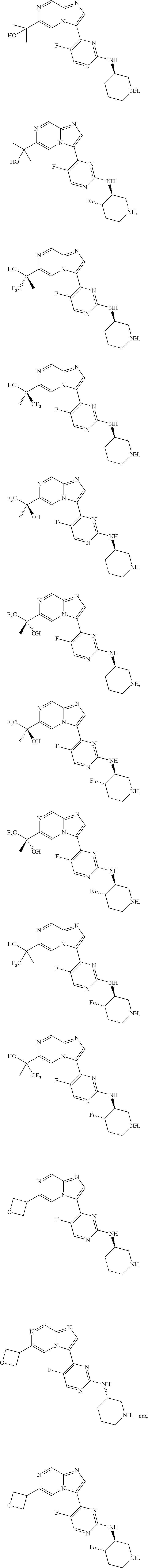

In one embodiment, (i) the compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof is an inhibitor of at least one of IRAK1, IRAK4, and FLT3; (ii) the compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof is an inhibitor of IRAK1 and IRAK4 and is not an inhibitor of FLT3; or (iii) the compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof is an inhibitor of each of IRAK1, IRAK4, and FLT3.

In another aspect, the present disclosure provides a composition comprising a compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof wherein the composition further comprises a formulary ingredient, an adjuvant, or a carrier.

In yet another aspect, the present disclosure provides a method of treating a disease or disorder in a subject in need thereof, the method comprising administering to the subject a therapeutically effective amount of a compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof or a composition comprising a compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof. In one embodiment, the disease or disorder is alleviated by inhibition of at least one of interleukin-1 receptor-associated kinase (IRAK) inhibition and fins-like tyrosine kinase 3 (FLT3) inhibition. In one embodiment, the disease or disorder comprises a hematopoietic cancer. In one embodiment, the hematopoietic cancer is selected from myelodysplastic syndrome (MDS), acute myeloid leukemia (AIL), lymphoma, leukemia, chronic lymphocytic leukemia (CLL), chronic myeloid leukemia (CML), acute lymphoblastic leukemia (ALL), non-Hodgkin lymphoma, Waldenstrom's macroglobulinemia, B cell lymphoma, diffuse large B-cell lymphoma (DLBCL), DLBCL with MYD88 mutation, follicular lymphoma, and marginal zone lymphoma. In one embodiment, the disease or disorder is selected from: BCL2 inhibitor resistant acute myeloid leukemia (AML), BCL2 inhibitor resistant refractory acute myeloid leukemia, BCL2 inhibitor resistant relapsed acute myeloid leukemia, FLT3 inhibitor resistant acute myeloid leukemia, FLT3 inhibitor resistant refractory acute myeloid leukemia, or FLT3 inhibitor resistant relapsed acute myeloid leukemia. In one embodiment, the disease or disorder comprises at least one cancer selected from bone marrow cancer, glioblastoma multiforme, endometrial cancer, melanoma, prostate cancer, lung cancer, breast cancer, kidney cancer, bladder cancer, basal cell carcinoma, thyroid cancer, squamous cell carcinoma, neuroblastoma, ovarian cancer, renal cell carcinoma, hepatocellular carcinoma, colon cancer, pancreatic cancer, rhabdomyosarcoma, meningioma, gastric cancer, Glioma, oral cancer, nasopharyngeal carcinoma, rectal cancer, stomach cancer, and uterine cancer. In one embodiment, the disease or disorder comprises at least one inflammatory disease or autoimmune disease selected from chronic inflammation, sepsis, rheumatoid arthritis, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, colitis, and atopic dermatitis. In one embodiment, the method further comprises administering to the subject one or more additional therapies selected from: a chemotherapy agent, a BCL2 inhibitor, an immune modulator, a BTK inhibitor, a DNA methyltransferase inhibitor/hypomethylating agent, an anthracycline, a histone deacetylase (HDAC) inhibitor, a purine nucleoside analogue (antimetabolite), an isocitrate dehydrogenase 1 or 2 (IDH1 and/or IDH2) inhibitor, an antibody-drug conjugate, an mAbs/immunotherapy, a Plk inhibitor, a MEK inhibitor, a CDK inhibitor, a CDK9 inhibitor, a CDK8 inhibitor, a retinoic acid receptor agonist, a TP53 activator, a CELMoD, a smoothened receptor antagonist, an ERK inhibitor including an ERK2/MAPK1 or ERK1/MAPK3 inhibitor, a PI3K inhibitor, an mTOR inhibitor, a steroid or glucocorticoid, a steroid or glucocorticoid receptor modulator, an EZH2 inhibitor, a hedgehog (Hh) inhibitor, a Topoisomerase I inhibitor, a Topoisomerase II inhibitor, an aminopeptidase/Leukotriene A4 hydrolase inhibitor, a FLT3/Axl/ALK inhibitor, a FLT3/KIT/PDGFR, PKC, and/or KDR inhibitor, a Syk inhibitor, an E-selectin inhibitor, an NEDD8-activator, an MDM2 inhibitor, a PLK1 inhibitor, an Aura A inhibitor, an aurora kinase inhibitor, an EGFR inhibitor, an AuroraB/C/VEGFR1/2/3/FLT3/CSF-1R/Kit/PDGFRA/B inhibitor, an AKT 1, 2, and/or 3 inhibitor, a ABL1/2/SRC/EPHA2/LCK/YES1/KIT/PDGFRB/FYN inhibitor, a farnesyltransferase inhibitor, a BRAF/MAP2K1/MAP2K2 inhibitor, a Menin-KMT2A/MLL inhibitor, and a multikinase inhibitor. In one embodiment, the additional therapy is at least one of a BCL2 inhibitor, a BTK inhibitor, a glucocorticoid, a CDK inhibitor, and a DNA methyltransferase inhibitor. In one embodiment, the BCL2 inhibitor is venetoclax or a pharmaceutically acceptable salt thereof, the BTK inhibitor is ibrutinib or a pharmaceutically acceptable salt thereof, the glucocorticoid is selected from dexamethasone, methylprednisolone, prednisolone, or a pharmaceutically acceptable salt of any one thereof, the CDK inhibitor is selected from CDK4/6 inhibitor palbociclib, CDK7 inhibitor THZ1, and/or CDK9 inhibitors BAY1251152 and atuveciclib, or a pharmaceutically acceptable salt of any one thereof, or the DNA methyltransferase inhibitor is azacitidine or a pharmaceutically acceptable salt thereof. In one embodiment, the compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof or the composition comprising a compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof and the one or more additional therapies are administered together in one administration or composition. In one embodiment, the compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof or the composition comprising a compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof and the one or more additional therapies are administered separately in more than one administration or more than one composition. In one embodiment, the compound is a compound of any one of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof.

In another aspect, the present disclosure provides a method of increasing survivability in a subject diagnosed with acute myeloid leukemia (AML) or suspected of having AML, the method comprising administering to the subject a therapeutically effective amount of a compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof or a composition comprising a compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof. In one embodiment, the survivability of the subject is increased compared to a subject treated with a therapeutically effective amount of the standard of care for AML. In one embodiment, the standard of care for AML comprises gilteritinib or a pharmaceutically acceptable salt thereof. In one embodiment, the subject is a human. In one embodiment, the survivability of the subject is increased by about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, or about 20 years compared to a subject treated with a therapeutically effective amount of the standard of care for AML. In one embodiment, the method comprises administering to the subject the therapeutically effective amount of a compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof or the composition comprising a compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof about every 6 hours, every 12 hours, every 18 hours, once a day, every other day, every 3 days, every 4 days, every 5 days, every 6 days, or once a week. In one embodiment, the method further comprises administering to the subject one or more additional therapies selected from: a chemotherapy agent, a BCL2 inhibitor, an immune modulator, a BTK inhibitor, a DNA methyltransferase inhibitor/hypomethylating agent, an anthracycline, a histone deacetylase (HDAC) inhibitor, a purine nucleoside analogue (antimetabolite), an isocitrate dehydrogenase 1 or 2 (IDH1 and/or IDH2) inhibitor, an antibody-drug conjugate, an mAbs/immunotherapy, a Plk inhibitor, a MWK inhibitor, a CDK inhibitor, a CDK9 inhibitor, a CDK8 inhibitor, a retinoic acid receptor agonist, a TP53 activator, a CELMoD, a smoothened receptor antagonist, an ERK inhibitor including an ERK2/MAPK1 or ERK1/MAPK3 inhibitor, a PI3K inhibitor, an mTOR inhibitor, a steroid or glucocorticoid, a steroid or glucocorticoid receptor modulator, an EZH2 inhibitor, a hedgehog (Hh) inhibitor, a Topoisomerase I inhibitor, a Topoisomerase II inhibitor, an aminopeptidase/Leukotriene A4 hydrolase inhibitor, a FLT3/Axl/ALK inhibitor, a FLT3/KIT/PDGFR, PKC, and/or KDR inhibitor, a Syk inhibitor, an E-selectin inhibitor, an NEDD8-activator, an MIDM2 inhibitor, a PLK1 inhibitor, an Aura A inhibitor, an aurora kinase inhibitor, an EGFR inhibitor, an AuroraB/C/VEGFR1/2/3/FLT3/CSF-1R/Kit/PDGFRA/B inhibitor, an AKT 1, 2, and/or 3 inhibitor, a ABL1/2/SRC/EPHA2/LCK/YES1/KIT/PDGFRB/FYN inhibitor, a farnesyltransferase inhibitor, a BRAF/MAP2K1/MAP2K2 inhibitor, a Menin-KMT2A/MLL inhibitor, and a multikinase inhibitor. In one embodiment, the additional therapy is at least one of a BCL2 inhibitor, a BTK inhibitor, a glucocorticoid, a CDK inhibitor, and a DNA methyltransferase inhibitor. In one embodiment, the BCL2 inhibitor is venetoclax or a pharmaceutically acceptable salt thereof, the BTK inhibitor is ibrutinib or a pharmaceutically acceptable salt thereof, the glucocorticoid is selected from dexamethasone, methylprednisolone, prednisolone, or a pharmaceutically acceptable salt of any one thereof, the CDK inhibitor is selected from CDK4/6 inhibitor palbociclib, CDK7 inhibitor THZ1, and/or CDK9 inhibitors BAY1251152 and atuveciclib, or a pharmaceutically acceptable salt of any one thereof, and the DNA methyltransferase inhibitor is azacitidine or a pharmaceutically acceptable salt thereof. In one embodiment, the AML is selected from: BCL2 inhibitor resistant AIL, BCL2 inhibitor resistant refractory AML, BCL2 inhibitor resistant relapsed AML, FLT3 inhibitor resistant AML, FLT3 inhibitor resistant refractory AMVL, or FLT3 inhibitor resistant relapsed AML. In one embodiment, the compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof or the composition comprising a compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof and the one or more additional therapies are administered together in one administration or composition. In one embodiment, the compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof or the composition comprising a compound of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof and the one or more additional therapies are administered separately in more than one administration or more than one composition.

### BRIEF DESCRIPTION OF THE DRAWINGS

FIG. 1 depicts the relative concentration (nM) of representative compounds of the disclosure, and gilteritinib (“Gil”), needed to fully potentiate (<20%) of the 312.5 nM Venetoclax Cell Titer Glo response in THP1 cells at 48 hours. A smaller concentration indicates higher potency to synergize with Venetoclax.

FIG. 2 depicts the relative concentration (nM) of representative compounds of the disclosure, and gilteritinib (“Gil”), needed to fully potentiate (<10%) of the 62.5 nM Venetoclax Cell Titer Glo response in MOLM14 (D835Y) cells at 48 hours. A smaller concentration indicates higher potency to synergize with Venetoclax.

FIG. 3 provides the structures of compounds studied in FIGS. 4 and 5A-5B.

FIG. 4 is a chart of survival of mice intravascularly engrafted with AML cells comparing the survival of Compound C (at 30 mg/kg) to control, gilteritinib SOC (at 30 mg/kg), and CA-4948 (Emavusertib, at 30 mg/kg).

FIGS. 5A-5B are charts depicting the leukemic grade at the time of necropsy. When adjusting for survival, all treated animals show significant improvement compared to controls and F06 has decreased scores compared to standard of care (F02). FIG. 5A: Leukemic grade at the time of necropsy (the statistical analysis used was mean with 95% confidence interval). FIG. 5B: Survival-adjusted leukemic grade at the time of necropsy (the statistical analysis used was geometric mean with geometric SD). All compounds used in this study were administered at 30 mg/kg.

### DETAILED DESCRIPTION OF THE INVENTION

The following applications are incorporated by reference herein in their entirety, and for all purposes: International Publication No. WO 2018081738, TREATMENT OF DISEASES ASSOCIATED WITH ACTIVATED IRAK, filed Oct. 30, 2017; U.S. Publication No. 2021/0292843, TREATMENT OF DISEASES ASSOCIATED WITH ACTIVATED IRAK, filed Apr. 4, 2019; International Publication No. WO 2014190163, Combination Therapy for MDS, filed May 22, 2014; U.S. Pat. No. 9,168,257, Combination Therapy for MDS, issued Oct. 27, 2015; U.S. Pat. No. 9,504,706, Combination Therapy for MDS, issued Nov. 29, 2016; U.S. Pat. No. 9,855,273, Combination Therapy for MDS, issued Jan. 2, 2018; International Publication No. WO 2018038988, Compounds, Compositions, Methods for Treating Diseases, and Methods for Preparing Compounds, filed Aug. 16, 2017; U.S. Pat. No. 11,254,667, Substituted imidazo[1,2-a]pyridines as IRAK 1/4 and FLT3 inhibitors, issued Feb. 2, 2022; U.S. Publication No. 2022/0213094, Substituted Imidazo[1,2-a]-pyridines as IRAK 1/4 and FLT3 Inhibitors, filed Jan. 4, 2022; U.S. Publication No. 2020/0199123, Substituted imidazo[1,2-a]pyridines as IRAK 1/4 and FLT3 inhibitors, filed Feb. 28, 2020; U.S. Publication No. 2022/0235042, Substituted Imidazo[1,2-a]-pyridines as IRAK 1/4 and FLT3 Inhibitors, filed Jan. 28, 2022; International Publication No. WO 2020252487, Rational therapeutic targeting of oncogenic immune signaling states in myeloid malignancies via the ubiquitin conjugating enzyme UBE2N, filed Jun. 15, 2020; International Publication No. WO 2022026935, Multi-Cyclic IRAK and FLT3 Inhibiting Compounds and Uses Thereof, filed Jul. 31, 2021; International Publication No. WO 2022140647, Multi-Cyclic IRAK and FLT3 Inhibiting Compounds and Uses Thereof, filed Dec. 23, 2021; International Publication No. WO 2023009833, Multi-Cyclic IRAK and FLT3 Inhibiting Compounds and Uses Thereof, filed Jul. 29, 2022; International Patent Application No. PCT/US2023/068520, Multi-Cyclic IRAK and FLT3 Inhibiting Compounds and Uses Thereof, filed Jun. 15, 2023; International Patent Application No. PCT/US2023/068897, Multi-Cyclic IRAK and FLT3 Inhibiting Compounds and Uses Thereof, filed Jun. 22, 2023; International Patent Application No. PCT/US2023/071435, Multi-Cyclic IRAK and FLT3 Inhibiting Compounds and Uses Thereof, filed Aug. 1, 2023; and International Patent Application No. PCT/US2023/034438, Multi-Cyclic IRAK and FLT3 Inhibiting Compounds and Uses Thereof, filed Oct. 4, 2023.

While embodiments encompassing the general inventive concepts may take diverse forms, various embodiments will be described herein, with the understanding that the present disclosure is to be considered merely exemplary, and the general inventive concepts are not intended to be limited to the disclosed embodiments.

Some embodiments of the invention include inventive compounds (e.g., compounds of Formula (I)). Other embodiments include compositions (e.g., pharmaceutical compositions) comprising the inventive compound. Still other embodiments of the invention include compositions for treating, for example, certain diseases using the inventive compounds. Some embodiments include methods of using the inventive compound (e.g., in compositions or in pharmaceutical compositions) for administering and treating. Further embodiments include methods for making the inventive compound. Yet further embodiments include methods for determining whether a particular patient is likely to be responsive to such treatment with the inventive compounds and compositions.

Unless otherwise noted, terms are to be understood according to conventional usage by those of ordinary skill in the relevant art.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

As used herein, in relation to compounds of Formulae (I), (II), (III), etc., the term “attached” signifies a stable covalent bond, certain preferred points of attachment being apparent to those of ordinary skill in the art.

As used herein (unless otherwise specified), the term “alkyl” means a monovalent, straight or branched hydrocarbon chain, which can be fully saturated, mono- or polyunsaturated and can include di- and multivalent radicals, having the number of carbon atoms designated (i.e., C₁-C₁₀ means one to ten carbons). For example, the terms “C₁-C₇ alkyl” or “C₁-C₄ alkyl” refer to straight- or branched-chain saturated hydrocarbon groups having from 1 to 7 (e.g., 1, 2, 3, 4, 5, 6, or 7), or 1 to 4 (e.g., 1, 2, 3, or 4), carbon atoms, respectively. Examples of C₁-C₇ alkyl groups include, but are not limited to, methyl, ethyl, n-propyl, i-propyl, n-butyl, s-butyl, t-butyl, n-pentyl, s-pentyl, n-hexyl, and n-heptyl. Examples of C₁-C₄ alkyl groups include, but are not limited to, methyl, ethyl, n-propyl, i-propyl, n-butyl, s-butyl, and t-butyl.

As used herein (unless otherwise specified), the term “alkenyl” means a monovalent, straight or branched hydrocarbon chain that includes one or more (e.g., 1, 2, 3, or 4) double bonds. Double bonds can occur in any stable point along the chain and the carbon-carbon double bonds can have either the cis or trans configuration. For example, this definition shall include but is not limited to ethenyl, propenyl, butenyl, pentenyl, hexenyl, heptenyl, octenyl, nonenyl, decenyl, undecenyl, 1,5-octadienyl, 1,4,7-nonatrienyl, cyclopentenyl, cyclohexenyl, cycloheptenyl, cyclooctenyl, ethylcyclohexenyl, butenylcyclopentyl, 1-pentenyl-3-cyclohexenyl, and the like. Similarly, “heteroalkenyl” refers to heteroalkyl having one or more double bonds. Further examples of alkenyl groups include, but are not limited to, vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, and 5-hexenyl.

As used herein (unless otherwise specified), the term “alkynyl” means a monovalent, straight or branched hydrocarbon chain that includes one or more (e.g., 1, 2, 3, or 4) triple bonds and that also may optionally include one or more (e.g. 1, 2, 3, or 4) double bonds in the chain. Examples of alkynyl groups include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl.

As used herein (unless otherwise specified), the term “alkoxy” means any of the above alkyl, alkenyl, or alkynyl groups which is attached to the remainder of the molecule by an oxygen atom (alkyl-O—). Examples of alkoxy groups include, but are not limited to, methoxy (sometimes shown as MeO—), ethoxy, isopropoxy, propoxy, and butyloxy.

The term “alkylene,” by itself or as part of another substituent, means, unless otherwise stated, a divalent radical derived from an alkyl, alkenyl, or alkynyl group, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the compounds disclosed herein. A “lower alkyl” or “lower alkylene” is a shorter chain alkyl or alkylene group, generally having eight or fewer carbon atoms.

As used herein (unless otherwise specified), the term “cycloalkyl” means a monovalent, monocyclic or bicyclic, 3, 4, 5, 6, 7, 8, 9, 10, 11, or 12 membered hydrocarbon group. The rings can be saturated or partially unsaturated. Examples of cycloalkyl groups include, but are not limited to, cyclopropyl, cyclobutyl, cyclopentyl, cyclohexyl, cycloheptyl, cyclooctyl, and bicycloalkyls (e.g., bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds). For a monocyclic cycloalkyl, the ring is not aromatic. For a bicyclic cycloalkyl, if one ring is aromatic, then the other is not aromatic. For a bicyclic cycloalkyl, one or both rings can be substituted.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms can optionally be oxidized, and the nitrogen heteroatom can optionally be quaternized. The heteroatom(s) O, N, P, S, and Si can be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms can be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R¹¹ or the like, it will be understood that the terms heteroalkyl and —NR′R¹¹ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

As used herein (unless otherwise specified), the term “halogen” or “halo” means monovalent Cl, F, Br, or I. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

As used herein (unless otherwise specified), the term “aryl” means a monovalent, monocyclic or bicyclic, 5, 6, 7, 8, 9, 10, 11, or 12 member aromatic hydrocarbon group and also means polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. Examples of aryl groups include, but are not limited to, phenyl, naphthyl, tolyl, and xylyl. For an aryl that is bicyclic, one or both rings can be substituted.

As used herein (unless otherwise specified), the term “heteroaryl” means a monovalent, monocyclic or bicyclic, 5, 6, 7, 8, 9, 10, 11, or 12 membered, hydrocarbon group, where 1, 2, 3, 4, 5, or 6 carbon atoms are replaced by a hetero atom independently selected from nitrogen, oxygen, or sulfur atom, and the monocyclic or bicyclic ring system is aromatic. Heteroaryl groups (or rings) can contain from one to four heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Examples of heteroaryl groups include, but are not limited to, thienyl (or thiophenyl), furyl, indolyl, pyrrolyl, pyridinyl, pyrazinyl, oxazolyl, thiaxolyl, quinolinyl, pyrimidinyl, imidazolyl, triazolyl, tetrazolyl, 1H-pyrazol-4-yl, 1-Me-pyrazol-4-yl, pyridin-3-yl, pyridin-4-yl, 3,5-dimethylisoxazolyl, 1H-pyrrol-3-yl, 3,5-di-Me-pyrazolyl, and 1H-pyrazol-4-yl. For a bicyclic heteroaryl, if one ring is aryl, then the other is heteroaryl. For a bicyclic heteroaryl, one or both rings can have one or more hetero atoms. For a bicyclic heteroaryl, one or both rings can be substituted.

An “arylene” and a “heteroarylene,” alone or as part of another substituent, mean a divalent radical derived from an aryl and heteroaryl, respectively. Accordingly, the term “aryl” can represent an unsubstituted, mono-, di- or trisubstituted monocyclic, polycyclic, biaryl and heterocyclic aromatic groups covalently attached at any ring position capable of forming a stable covalent bond, certain preferred points of attachment being apparent to those skilled in the art (e. g. 3-indolyl, 4-imidazolyl). The aryl substituents are independently selected from the group consisting of halo, nitro, cyano, trihalomethyl, C₁₋₁₆alkyl, arylC₁₋₁₆alkyl, C₀₋₁₆alkyloxyC₀₋₁₆alkyl, arylC₀₋₁₆alkyloxyC₀₋₁₆alkyl, C₀₋₁₆alkylthioC₀₋₁₆alkyl, arylC₀₋₁₆alkylthioC₀₋₁₆alkyl, C₀₋₁₆alkylaminoC₀₋₁₆alkyl, arylC₀₋₁₆alkylaminoC₀₋₁₆alkyl, di(arylC₁₋₁₆alkyl)aminoC₀₋₁₆alkyl, C₁₋₁₆alkylcarbonylC₀₋₁₆alkyl, arylC₁₋₁₆alkylcarbonylC₀₋₁₆alkyl, C₁₋₁₆alkylcarboxyC₀₋₁₆alkyl, arylC₁₋₁₆alkylcarboxyC₀₋₁₆alkyl, C₁₋₁₆alkylcarbonylaminoC₀₋₁₆alkyl, arylC₁₋₁₆alkylcarbonylaminoC₀₋₁₆alkyl, —C₀₋₁₆alkylCOOR₄, —C₀₋₁₆alkylCONR₅R₆ wherein R₄, R₅ and R₆ are independently selected from hydrogen, C₁-C₁₁alkyl, arylC₀-C₁₁alkyl, or R₅ and R₆ are taken together with the nitrogen to which they are attached forming a cyclic system containing 3 to 8 carbon atoms with or without one C₁₋₁₆alkyl, arylC₀-C₁₆alkyl, or C₀-Cl₁₆alkylaryl substituent. Aryl includes but is not limited to pyrazolyl and triazolyl.

For brevity, the term “aryl” when used in combination with other terms (e.g., aryloxy, arylthioxy, arylalkyl) includes both aryl and heteroaryl rings as defined above. Thus, the terms “arylalkyl,” “aralkyl” and the like are meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like), or a sulfur atom. Accordingly, the terms “arylalkyl” and the like (e.g. (4-hydroxyphenyl)ethyl, (2-aminonaphthyl)hexyl, pyridylcyclopentyl) represents an aryl group as defined above attached through an alkyl group as defined above having the indicated number of carbon atoms.

The terms “cycloalkyl” and “heterocycloalkyl”, also referred to as “heterocyclyl”, by themselves or in combination with other terms, mean, unless otherwise stated, cyclic versions of “alkyl” and “heteroalkyl,” respectively. Examples of cycloalkyl include, but are not limited to, cyclopropyl, cyclobutyl, cyclopentyl, cyclohexyl, 1-cyclohexenyl, 3-cyclohexenyl, cycloheptyl, and the like. As used herein (unless otherwise specified), the term “heterocycloalkyl” or “heterocyclyl” means a monovalent, monocyclic or bicyclic, 5, 6, 7, 8, 9, 10, 11, or 12 membered, hydrocarbon, where 1, 2, 3, 4, 5, or 6 carbon atoms are replaced by a hetero atom independently selected from nitrogen atom, oxygen atom, or sulfur atom, and the monocyclic or bicyclic ring system is not aromatic. Additionally, for heterocycloalkyl, a heteroatom can occupy the position at which the heterocycle is attached to the remainder of the molecule. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, tetrahydropyran, pyrolidinyl (e.g., pyrrolidin-1-yl, pyrrolidin-2-yl, pyrrolidin-3-yl, or pyrrolidin-4-yl), piperazinyl (e.g., piperazin-1-yl, piperazin-2-yl, piperazin-3-yl, or piperazin-4-yl), piperidinyl (e.g., piperadin-1-yl, piperadin-2-yl, piperadin-3-yl, or piperadin-4-yl), and morpholinyl (e.g., morpholin-1-yl, morpholin-2-yl, morpholin-3-yl, or morpholin-4-yl). For a bicyclic heterocyclyl, if one ring is aromatic (e.g., monocyclic aryl or heteroaryl), then the other ring is not aromatic. For a bicyclic heterocyclyl, one or both rings can have one or more hetero atoms. For a bicyclic heterocyclyl, one or both rings can be substituted and the like. A “cycloalkylene” and a “heterocycloalkylene,” alone or as part of another substituent, means a divalent radical derived from a cycloalkyl and heterocycloalkyl, respectively.

As used herein (unless otherwise specified), the term “hetero atom” means an atom selected from nitrogen atom, oxygen atom, or sulfur atom.

As used herein (unless otherwise specified), the terms “hydroxy” or “hydroxyl” means a monovalent —OH group.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ can have a specified number of carbons (e.g., “C₁-C₄ alkylsulfonyl”).

The term “carbonyloxy” represents a carbonyl group attached through an oxygen bridge.

In the above definitions, the terms “alkyl” and “alkenyl” can be used interchangeably in so far as a stable chemical entity is formed, as would be apparent to those skilled in the art.

The term “linker” refers to attachment groups interposed between substituents. In some embodiments, the linker includes amido (—CONH—Rⁿ or —NHCO—Rⁿ), thioamido (—CSNH—Rⁿ or —NHCS—Rⁿ), carboxyl (—CO₂—Rⁿ or —OCORⁿ), carbonyl (—CO—Rⁿ), urea (—NHCONH—Rⁿ), thiourea (—NHCSNH—Rⁿ), sulfonamido (—NHSO₂—Rⁿ or —SO₂NH—Rⁿ), ether (—O—Rⁿ), sulfonyl (—SO₂—Rⁿ), sulfoxyl (—SO—Rⁿ), carbamoyl (—NHCO₂—Rⁿ or —OCONH—Rⁿ), or amino (—NHRⁿ) linking moieties.

Each of the above terms (e.g., “alkyl,” “heteroalkyl,” “aryl,” and “heteroaryl”, and so forth) includes both substituted and unsubstituted forms of the indicated radical. Preferred substituents for each type of radical are provided herein.

As used herein (unless otherwise specified), the term “substituted” (e.g., as in substituted alkyl) means that one or more hydrogen atoms of a chemical group (with one or more hydrogen atoms) can be replaced by one or more non-hydrogen substituents selected from the specified options. The replacement can occur at one or more positions. The term “optionally substituted” means that one or more hydrogen atoms of a chemical group (with one or more hydrogen atoms) can be, but is not required to be substituted.

A “substituent group,” as used herein, means a non-hydrogen substituent group that may be, and preferably is, a group selected from the following moieties:
(A) —NH₂, —SH, —CN, —CF₃, —NO₂, halogen, hydroxy, oxo, —CN, methanoyl (—COH), carboxy (—CO₂H), nitro (—NO₂), —N(CH₃)₂, ethynyl (—CCH), propynyl, sulfo (—SO₃H), CONH₂, —CON(CH₃)₂, unsubstituted C₁-C₇ alkyl, unsubstituted C₁-C₇ heteroalkyl, unsubstituted C₁-C₇ perfluorinated alkyl, unsubstituted C₁-C₇ alkoxy, unsubstituted C₁-C₇ haloalkoxy, unsubstituted cycloalkyl, unsubstituted heterocycloalkyl, unsubstituted aryl, unsubstituted heteroaryl, and
(B) C₁-C₇ alkyl, C₁-C₇ heteroalkyl, C₁-C₇ perfluorinated alkyl, C₁-C₇ alkoxy, C₁-C₇ haloalkoxy, cycloalkyl, heterocycloalkyl, aryl, and heteroaryl, substituted with at least one substituent selected from:
(i) —NH₂, —SH, —CN, —CF₃, —NO₂, halogen, hydroxy, oxo, —CN, methanoyl (—COH), carboxy (—CO₂H), nitro (—NO₂), —N(CH₃)₂, ethynyl (—CCH), propynyl, sulfo (—SO₃H), CONH₂, —CON(CH₃)₂, unsubstituted C₁-C₇ alkyl, unsubstituted C₁-C₇ heteroalkyl, unsubstituted C₁-C₇ perfluorinated alkyl, unsubstituted C₁-C₇ alkoxy, unsubstituted C₁-C₇ haloalkoxy, unsubstituted cycloalkyl, unsubstituted heterocycloalkyl, unsubstituted aryl, unsubstituted heteroaryl, and
(ii) C₁-C₇ alkyl, C₁-C₇ heteroalkyl, C₁-C₇ perfluorinated alkyl, C₁-C₇ alkoxy, C₁-C₇ haloalkoxy, cycloalkyl, heterocycloalkyl, aryl, and heteroaryl, substituted with at least one substituent selected from:
(a) —NH₂, —SH, —CN, —CF₃, —NO₂, halogen, hydroxy, oxo, —CN, methanoyl (—COH), carboxy (—CO₂H), nitro (—NO₂), —N(CH₃)₂, ethynyl (—CCH), propynyl, sulfo (—SO₃H), CONH₂, —CON(CH₃)₂, unsubstituted C₁-C₇ alkyl, unsubstituted C₁-C₇ heteroalkyl, unsubstituted C₁-C₇ perfluorinated alkyl, unsubstituted C₁-C₇ alkoxy, unsubstituted C₁-C₇ haloalkoxy, unsubstituted cycloalkyl, unsubstituted heterocycloalkyl, unsubstituted aryl, unsubstituted heteroaryl, and
(b) C₁-C₇ alkyl, C₁-C₇ heteroalkyl, C₁-C₇ perfluorinated alkyl, C₁-C₇ alkoxy, C₁-C₇ haloalkoxy, cycloalkyl, heterocycloalkyl, aryl, and heteroaryl, substituted with at least one substituent selected from: —NH₂, —SH, —CN, —CF₃, —NO₂, halogen, hydroxy, oxo, —CN, methanoyl (—COH), carboxy (—CO₂H), nitro (—NO₂), —N(CH₃)₂, ethynyl (—CCH), propynyl, sulfo (—SO₃H), CONH₂, —CON(CH₃)₂, unsubstituted C₁-C₇ alkyl, unsubstituted C₁-C₇ heteroalkyl, unsubstituted C₁-C₇ perfluorinated alkyl, unsubstituted C₁-C₇ alkoxy, unsubstituted C₁-C₇ haloalkoxy, unsubstituted cycloalkyl, unsubstituted heterocycloalkyl, unsubstituted aryl, unsubstituted heteroaryl.

A “size-limited substituent” or “size-limited substituent group,” as used herein, means a group, e.g., selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2-20-membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4-8-membered heterocycloalkyl.

A “lower substituent” or “lower substituent group,” as used herein, means a group, e.g., selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2-8-membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5-7-membered heterocycloalkyl.

The term “about” used in the context of a numeric value indicates a range of +/−10% of the numeric value, unless expressly indicated otherwise.

Some compounds of the invention can have one or more chiral centers and can exist in and be isolated in optically active and racemic forms, for any of the one or more chiral centers. Some compounds can exhibit polymorphism. The compounds of the present invention (e.g., Formula I) encompass any optically active, racemate, stereoisomer form, polymorphism, or mixtures thereof. If a chiral center does not provide an indication of its configuration (i.e., R or S) in a chemical structure, it should be considered to represent R, S or a racemate.

As used herein, the term “sample” encompasses a sample obtained from a subject or patient. The sample can be of any biological tissue or fluid. Such samples include, but are not limited to, sputum, saliva, buccal sample, oral sample, blood, serum, mucus, plasma, urine, blood cells (e.g., white cells), circulating cells (e.g. stem cells or endothelial cells in the blood), tissue, core or fine needle biopsy samples, cell-containing body fluids, free floating nucleic acids, urine, stool, peritoneal fluid, and pleural fluid, tear fluid, or cells therefrom. Samples can also include sections of tissues such as frozen or fixed sections taken for histological purposes or microdissected cells or extracellular parts thereof. A sample to be analyzed can be tissue material from a tissue biopsy obtained by aspiration or punch, excision or by any other surgical method leading to biopsy or resected cellular material. Such a sample can comprise cells obtained from a subject or patient. In some embodiments, the sample is a body fluid that include, for example, blood fluids, serum, mucus, plasma, lymph, ascitic fluids, gynecological fluids, or urine but not limited to these fluids. In some embodiments, the sample can be a non-invasive sample, such as, for example, a saline swish, a buccal scrape, a buccal swab, and the like.

As used herein, “blood” can include, for example, plasma, serum, whole blood, blood lysates, and the like.

As used herein, the term “assessing” includes any form of measurement, and includes determining if an element is present or not. The terms “determining,” “measuring,” “evaluating,” “assessing,” “analyzing,” and “assaying” can be used interchangeably and can include quantitative and/or qualitative determinations.

As used herein, the term “monitoring” with reference to a type of cancer refers to a method or process of determining the severity or degree of the type of cancer or stratifying the type of cancer based on risk and/or probability of mortality. In some embodiments, monitoring relates to a method or process of determining the therapeutic efficacy of a treatment being administered to a patient.

As used herein, “outcome” can refer to an outcome studied. In some embodiments, “outcome” can refer to survival/mortality over a given time horizon. For example, “outcome” can refer to survival/mortality over 1 month, 3 months, 6 months, 1 year, 5 years, or 10 years or longer. In some embodiments, an increased risk for a poor outcome indicates that a therapy has had a poor efficacy, and a reduced risk for a poor outcome indicates that a therapy has had a good efficacy.

As used herein, the term “high risk clinical trial” refers to one in which the test agent has “more than minimal risk” (as defined by the terminology used by institutional review boards, or IRBs). In some embodiments, a high risk clinical trial is a drug trial.

As used herein, the term “low risk clinical trial” refers to one in which the test agent has “minimal risk” (as defined by the terminology used by IRBs). In some embodiments, a low risk clinical trial is one that is not a drug trial. In some embodiments, a low risk clinical trial is one that that involves the use of a monitor or clinical practice process. In some embodiments, a low risk clinical trial is an observational clinical trial.

As used herein, the terms “modulated” or “modulation,” or “regulated” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

As used herein, the term “subject” refers to any suitable (e.g., treatable) member of the animal kingdom. In the methods, the subject is preferably a mammal. In the methods, the subject is preferably a human patient. In the methods, the subject may be a mammalian pediatric patient. In the methods, the pediatric patient is a mammalian (e.g., preferably human) patient under 18 years of age, while an adult patient is 18 or older.

As used herein, the term “treating” (and its variations, such as “treatment” “treating,” “treat,” and the like) is, unless stated otherwise, to be considered in its broadest context and refers to obtaining a desired pharmacologic and/or physiologic effect. In particular, for example, the term “treating” may not necessarily imply or require that an animal is treated until total recovery. Accordingly, “treating” includes amelioration of the symptoms, relief from the symptoms or effects associated with a condition, decrease in severity of a condition, or preventing, preventively ameliorating symptoms, or otherwise reducing the risk of developing a particular condition. In some aspects, “treating” may not require or include prevention. As used herein, reference to “treating” an animal includes but is not limited to prophylactic treatment and therapeutic treatment. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a subject, preferably in a mammal (e.g., in a human), and may include one or more of: (a) preventing the disease from occurring in a subject which may be predisposed to the disease but has not yet been diagnosed as having it; (b) inhibiting the disease, i.e., arresting its development; and (c) relieving the disease, i.e., causing regression or elimination of the disease and/or relieving one or more disease symptoms. In particular aspects of the methods, such as conditions or disorders characterized by dysregulated IRAK expression or dysregulated (e.g., hyperactive) IRAK-mediated signaling pathway(s), treatment may be or include reducing such expression or signaling. “Treatment” can also encompass delivery of an agent or administration of a therapy in order to provide for a pharmacologic effect, even in the absence of a disease or condition. Any of the compositions (e.g., pharmaceutical compositions) described herein can be used to treat a suitable subject.

“Therapeutically effective amount” means an amount effective to achieve a desired and/or beneficial effect. An effective amount can be administered in one or more administrations. In the methods, a therapeutically effective amount is an amount appropriate to treat an indication. By treating an indication is meant achieving any desirable effect, such as one or more of palliate, ameliorate, stabilize, reverse, slow, or delay disease progression, increase the quality of life, or to prolong life. Such achievement can be measured by any suitable method, such as measurement of tumor size or blood cell count, or any other suitable measurement.

As used herein, the term “marker” or “biomarker” refers to a biological molecule, such as, for example, a nucleic acid, peptide, protein, hormone, and the like, whose presence or concentration can be detected and correlated with a known condition, such as a disease state. It can also be used to refer to a differentially expressed gene whose expression pattern can be utilized as part of a predictive, prognostic or diagnostic process in healthy conditions or a disease state, or which, alternatively, can be used in methods for identifying a useful treatment or prevention therapy.

As used herein, an mRNA “isoform” is an alternative transcript for a specific mRNA or gene. This term includes pre-mRNA, immature mRNA, mature mRNA, cleaved or otherwise truncated, shortened, or aberrant mRNA, modified mRNA (e.g. containing any residue modifications, capping variants, polyadenylation variants, etc.), and the like.

“Antibody” or “antibody peptide(s)” refer to an intact antibody, or a binding fragment thereof that competes with the intact antibody for specific binding; this definition also encompasses monoclonal and polyclonal antibodies. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody, for example, substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

Embodiments of the invention set forth herein include inventive compounds (e.g., compounds of Formula (I), such as compounds of Formula (II) and Formula (III)). Other embodiments include compositions (e.g., pharmaceutical compositions) comprising the inventive compound. Still other embodiments of the invention include compositions (e.g., pharmaceutical compositions) for treating, for example, certain diseases using the inventive compounds. Some embodiments include methods of using the inventive compound (e.g., in compositions or in pharmaceutical compositions) for administering and treating (e.g., diseases such as cancer or blood disorders). Some embodiments include methods of determining whether a patient is suitable for, or likely to respond favorably to, a particular treatment. Further embodiments include methods for making the inventive compounds. Additional embodiments of the invention are also discussed herein.

### Compounds and Compositions, Including Pharmaceutical Compositions

In one aspect, the present disclosure relates to a compound of Formula (I), (II), or (III):

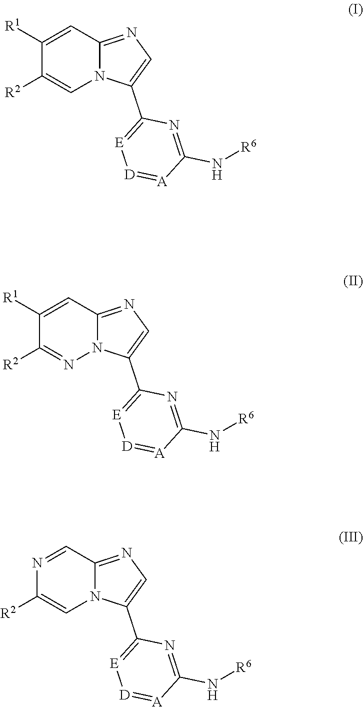

or a salt, ester, solvate, optical isomer, geometric isomer, salt of an isomer, prodrug, or derivative thereof. In an embodiment, the compound is a pharmaceutically acceptable salt, ester, solvate, optical isomer, geometric isomer, salt of an isomer, prodrug, or derivative of a compound of Formula (I), (II), or (III). In some embodiments, the compound is not an ester, not a solvate, and not a prodrug of a compound of Formula (I), (II), or (III).

In an embodiment, A of Formula (I), (II), or (III) is selected from N and CR⁵. In an embodiment, D of Formula (I), (II), or (III) is selected from N and CR⁴. In an embodiment, E of Formula (I), (II), or (III) is selected from N and CR³. In an embodiment, one of A, D, or E is N. In another embodiment, A is CR⁵, D is CR⁴, and E is CR³.

In exemplary embodiments, R¹, R², R³, R⁴, and R⁵ of Formula (I), (II), or (III) are each independently selected from H, halogen, hydroxy, oxo, —CN, amido, methanoyl (—COH), carboxy (—CO₂H), C₁-C₇ alkyl, C₂-C₇ alkenyl, C₂-C₇ alkynyl, C₁-C₇ heteroalkyl, C₁-C₇ alkoxy, —C(═O)NR³¹R³², cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl, wherein the amido, methanoyl (—COH), carboxy (—CO₂H), C₁-C₇ alkyl, C₂-C₇ alkenyl, C₂-C₇ alkynyl, C₂-C₆ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl is optionally substituted with one or more of deuterium, halogen, hydroxy, oxo, methanoyl (—COH), carboxy (—CO₂H), nitro (—NO₂), —NH₂, —NHCH₃, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CONHCH₃, —CON(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl.

In some embodiments of Formula (I), (II), or (III), R¹ can be H, deuterium, halogen, hydroxy, oxo, —CN, amido, methanoyl (—COH), carboxy (—CO₂H), C₁-C₇ alkyl, C₂-C₇ alkenyl, C₂-C₇ alkynyl, C₁-C₇ heteroalkyl, C₁-C₇ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl, wherein the amido, methanoyl (—COH), carboxy (—CO₂H), C₁-C₇ alkyl, C₂-C₇ alkenyl, C₂-C₇ alkynyl, C₂-C₆ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl is optionally substituted with one or more of deuterium, halogen, hydroxy, oxo, methanoyl (—COH), carboxy (—CO₂H), nitro (—NO₂), —NH₂, —NHCH₃, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CONHCH₃, —CON(CH₃)₂, C₁-C₇ alkyl, C₁-C₇ heteroalkyl, C₁-C₇ haloalkyl, C₁-C₇ perfluorinated alkyl, C₁-C₇ alkoxy, C₁-C₇ haloalkoxy, or C₁-C₇ alkyl which is substituted with cycloalkyl; R² can be H, deuterium, halogen, hydroxy, oxo, —CN, amino, methanoyl (—COH), carboxy (—CO₂H), C₁-C₇ alkyl, C₂-C₇ alkenyl, C₂-C₇ alkynyl, C₁-C₇ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl, wherein the amino, methanoyl (—COH), carboxy (—CO₂H), C₁-C₇ alkyl, C₂-C₇ alkenyl, C₂-C₇ alkynyl, C₁-C₇ heteroalkyl, C₁-C₇ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl is optionally substituted with one or more of deuterium, halogen, hydroxy, oxo, methanoyl (—COH), carboxy (—CO₂H), nitro (—NO₂), —NH₂, —NHCH₃, —N(CH₃)₂, cyano (—CN), ethynyl (—CCH), propynyl, sulfo (—SO₃H), heteroaryl, pyrrolyl, piperidyl, piperazinyl, morpholinyl, —CO-morpholin-4-yl, —CONH₂, —CONHCH₃, —CON(CH₃)₂, C₁-C₇ alkyl, C₁-C₇ heteroalkyl, C₁-C₇ haloalkyl, C₁-C₇ perfluorinated alkyl, C₁-C₇ alkoxy, C₁-C₇ haloalkoxy, cycloalkyl, heterocyclyl, spiro-fused cycloalkyl, aryl, fused ring aryl, heteroaryl, fused ring heteroaryl, or C₁-C₇ alkyl which is substituted with cycloalkyl; R³, R⁴, and R⁵ can be H, deuterium, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl, wherein the methanoyl (—COH), carboxy (—CO₂H), C₁-C₇ alkyl, C₂-C₇ alkenyl, C₂-C₇ alkynyl, C₁-C₇ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl is optionally substituted with one or more of deuterium, halogen, hydroxy, oxo, methanoyl (—COH), carboxy (—CO₂H), nitro (—NO₂), —NH₂, —NHCH₃, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CONHCH₃, —CON(CH₃)₂, C₁-C₇ alkyl, C₁-C₇ haloalkyl, C₁-C₇ perfluorinated alkyl, C₁-C₇ alkoxy, C₁-C₇ haloalkoxy, or C₁-C₇ alkyl which is substituted with cycloalkyl.

R⁶ of Formula (I), (II), or (III) can be

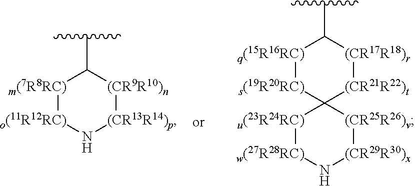

R⁷, R⁸, R⁹, R¹⁰, R¹¹, R¹², R¹³, R¹⁴ can be H, deuterium, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, —O-cycloalkyl, spiro-fused cycloalkyl, —O-spiro-fused cycloalkyl, heterocyclyl, —O— heterocyclyl, aryl, —O-aryl, heteroaryl, —O-heteroaryl, fused ring heteroaryl, or —O-fused ring heteroaryl, wherein the methanoyl (—COH), carboxy (—CO₂H), C₁-C₇ alkyl, C₂-C₇ alkenyl, C₂-C₇ alkynyl, C₁-C₇ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl is optionally substituted with one or more of deuterium, halogen, hydroxy, oxo, —C(═O)H, —C(═O)OH, nitro (—NO₂), —NH₂, —N(CH₃)₂, cyano (—CN), ethynyl (—CCH), propynyl, —SO₃H, cycloalkyl, heterocyclyl, aryl, heteroaryl, pyrrolyl, piperidyl, piperazinyl, morpholinyl, —C(═O)-morpholin-4-yl, —C(═O)NH₂, —C(═O)N(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl; R¹⁵, R¹⁶, R¹⁷, R¹⁸, R¹⁹, R²⁰, R²¹, R²², R²³, R²⁴, R²⁵, R²⁶, R²⁷, R²⁹, R²⁹, and R³⁰ can be H, deuterium, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl, wherein the methanoyl (—COH), carboxy (—CO₂H), C₁-C₇ alkyl, C₂-C₇ alkenyl, C₂-C₇ alkynyl, C₁-C₇ alkoxy, cycloalkyl, —O-cycloalkyl, spiro-fused cycloalkyl, —O-spiro-fused cycloalkyl, heterocyclyl, —O-heterocyclyl, aryl, —O-aryl, heteroaryl, —O-heteroaryl, fused ring heteroaryl, or —O-fused ring heteroaryl, is optionally substituted with one or more of deuterium, halogen, hydroxy, oxo, —C(═O)H, —C(═O)OH, nitro (—NO₂), —NH₂, —N(CH₃)₂, cyano (—CN), ethynyl (—CCH), propynyl, —SO₃H, cycloalkyl, heterocyclyl, aryl, heteroaryl, pyrrolyl, piperidyl, piperazinyl, morpholinyl, —C(═O)-morpholin-4-yl, —C(═O)NH₂, —C(═O)N(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl; and m, n, o, p, q, r, s, t, u, v, w, and x can be 0, 1, 2, 3, 4, or 5, where q+r+s+t is at least 1, and where u+v+w+x is at least 1.

In some embodiments, R¹ of Formula (I), (II), or (III) is H, deuterium, halogen, —CONH₂, —CONHCH₃, —CON(CH₃)₂, benzyl, C₁-C₇ alkyl, C₁-C₇ alkoxy, cycloalkyl, or —O-cycloalkyl, wherein the C₁-C₇ alkyl, C₁-C₇ alkoxy, cycloalkyl, or —O-cycloalkyl is optionally substituted with one or more deuterium, halogen, hydroxyl, C₁-C₇ alkyl, or C₁-C₇ haloalkyl. In some embodiments, R¹ is H, C₁, —CONH₂, —CONHCH₃, methoxy, —OCD₃, ethoxy, cyclopropyl, C₁-C₄ alkyl, and —O—C₃ cycloalkyl, wherein the methoxy, ethoxy, cyclopropyl, C₁-C₄ alkyl, or —O—C₃ cycloalkyl is optionally substituted with one or more F, —OH, methyl, or CF₃. In some embodiments, R¹ is H. In some embodiments, R¹ is not H.

In some embodiments, R² of Formula (I), (II), or (III) is H, deuterium, halogen, hydroxy, O-aryl, amino, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, fused ring aryl, heteroaryl, or fused ring heteroaryl, wherein the O-aryl, amino, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, fused ring aryl, heteroaryl, or fused ring heteroaryl is optionally substituted with one or more of deuterium, halogen, hydroxy, —CN, amino, cycloalkyl, heterocyclyl, aryl, heteroaryl, fused ring aryl, fused ring heteroaryl, pyrrolyl, piperidyl, piperazinyl, C₁-C₇ alkyl, C₁-C₇ haloalkyl, C₁-C₇ perfluorinated alkyl, C₁-C₇ alkoxy, C₁-C₇ haloalkoxy, or C₁-C₇ alkyl which is substituted with cycloalkyl. In some embodiments, R² is H, halogen, hydroxy, O-aryl, amino, C₁-C₇ alkyl, C₁-C₇ alkoxy, cycloalkyl, spiro-fused cycloalkyl, heterocyclyl, aryl, fused ring aryl, heteroaryl, or fused ring heteroaryl wherein the O-aryl, amino, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more of halogen, hydroxy, amino, cycloalkyl, heterocyclyl, aryl, heteroaryl, pyrrolyl, piperidyl, piperazinyl, C₁-C₇ alkyl, C₁-C₇ haloalkyl, C₁-C₇ perfluorinated alkyl, C₁-C₇ alkoxy, C₁-C₇ haloalkoxy, or C₁-C₇ alkyl which is substituted with cycloalkyl. In some embodiments, R² is H, C₁, hydroxy, —NHCH₃, —N(CH₃)₂, —OCH₃, —OCF₃, —OCHF₂, —OPh, —CF₃, —CHF₂, unsubstituted C₁-C₇ alkyl, substituted amino, substituted C₁-C₇ alkyl, substituted cycloalkyl, unsubstituted cycloalkyl, unsubstituted heterocyclyl, substituted heterocyclyl, substituted pyrazolyl, substituted fused ring heteroaryl, or unsubstituted fused ring heteroaryl. In some embodiments, R² is not H.

In some embodiments, R³ of Formula (I), (II), or (III) is H, deuterium, halogen, hydroxy, —CN, methanoyl (—COH), carboxy (—CO₂H), C₁-C₇ alkyl, or C₁-C₇ alkoxy, wherein the C₁-C₇ alkyl, or C₁-C₇ alkoxy is optionally substituted with one or more of deuterium, halogen, hydroxy, methanoyl (—COH), carboxy (—CO₂H), nitro (—NO₂), —NH₂, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CONHCH₃, —CON(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl. In some embodiments, R³ is H, halogen, hydroxy, —CN, methyl, —CF₃, —OCD₃, or methoxy. In some embodiments, R³ is H. In some embodiments, R³ is not H.

In some embodiments, R⁴ of Formula (I), (II), or (III) is H, deuterium, halogen, hydroxy, —CN, methanoyl (—COH), carboxy (—CO₂H), C₁-C₇ alkyl, or C₁-C₇ alkoxy, wherein the C₁-C₇ alkyl or C₁-C₇ alkoxy is optionally substituted with one or more of deuterium, halogen, hydroxy, methanoyl (—COH), carboxy (—CO₂H), nitro (—NO₂), —NH₂, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CONHCH₃, —CON(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl. In some embodiments, R⁴ is H, halogen, hydroxy, —CN, methyl, —CF₃, —OCD₃, or methoxy. In some embodiments, R⁴ is H. In some embodiments, R⁴ is not H.

In some embodiments, R⁵ of Formula (I), (II), or (III) is H, deuterium, halogen, hydroxy, —CN, methanoyl (—COH), carboxy (—CO₂H), C₁-C₇ alkyl, or C₁-C₇ alkoxy, wherein the C₁-C₇ alkyl or C₁-C₇ alkoxy is optionally substituted with one or more of halogen, hydroxy, methanoyl (—COH), carboxy (—CO₂H), nitro (—NO₂), —NH₂, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CONHCH₃, —CON(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl. In some embodiments, R⁵ is H, halogen, hydroxy, —CN, methyl, —CF₃, or methoxy.

In some embodiments, R⁴ of Formula (I), (II), or (III) is methyl or —CF₃, and at least one of R³ and R⁵ is H or halogen.

In some embodiments of Formula (I), (II), or (III), there is a chiral center at the R⁶ attachment carbon. In some embodiments, the chiral center is an R chiral center, an S chiral center, or a racemate. In certain embodiments, the chiral center can be represented by the following bonds [symbol] , [symbol] , [symbol] , [symbol] , or [symbol] . Where a chiral center is possible at other positions of the compounds according to Formula (I), (II), or (III), as would appreciated by one skilled in the art, the straight bond shown can also be can be [symbol] , [symbol] , [symbol] , [symbol] , or [symbol] .

In some embodiments, R⁶ of Formula (I), (II), or (III) is

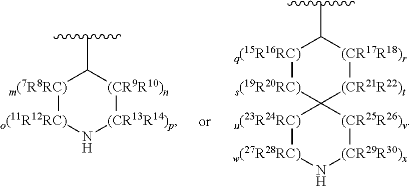

In some embodiments, R⁷, R⁸, R⁹, R¹⁰, R¹¹, R¹², R¹³, R¹⁴, if present, are independently selected from H, deuterium, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, —C(═O)NR³¹R³² cycloalkyl, spiro-fused cycloalkyl, heterocyclyl, aryl, heteroaryl, or fused ring heteroaryl, wherein the methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more deuterium, halogen, hydroxy, oxo, methanoyl (—COH), carboxy (—CO₂H), nitro (—NO₂), —NH₂, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CONHCH₃, —CON(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl. In one embodiment, at least one of R⁷, R⁸, R⁹, R¹⁰, R¹¹, R¹², R¹³, and R¹⁴ is not H. In one embodiment, each of R⁷, R⁸, R⁹, R¹⁰, R¹¹, R¹², R¹³, and R¹⁴, if present, is H.

In some embodiments, R¹⁵, R¹⁶, R¹⁷, R¹⁸, R¹⁹, R²⁰, R²¹, R²², R²³, R²⁴, R²⁵, R²⁶, R²⁷, R²⁹, R²⁹, and R³⁰, if present, are independently selected from H, deuterium, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein the methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more deuterium, halogen, hydroxy, oxo, methanoyl (—COH), carboxy (—CO₂H), nitro (—NO₂), —NH₂, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CONHCH₃, —CON(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl. In one embodiment, at least one of R¹⁵, R¹⁶, R¹⁷, R¹⁸, R¹⁹, R²⁰, R²¹, R²², R²³, R²⁴, R²⁵, R²⁶, R²⁷, R²⁹, R²⁹, and R³⁰ is not H. In another embodiment, each of R¹⁵, R¹⁶, R¹⁷, R¹⁸, R¹⁹, R²⁰, R²¹, R²², R²³, R²⁴, R²⁵, R²⁶, R²⁷, R²⁹, R²⁹, and R³⁰, if present, is H.

In some embodiments, m, n, o, p, q, r, s, t, u, v, w, and x are independently selected from 0, 1, 2, 3, 4, or 5, where q+r+s+t is at least 1, and where u+v+w+x is at least 1.

In some embodiments, R⁶ of Formula (I), (II), or (III) is

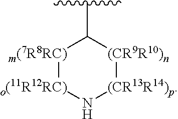

In some embodiments, R⁶ of Formula (I), (II), or (III) is

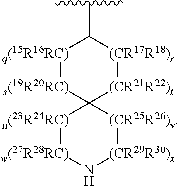

In some embodiments, R⁷, R⁸, R⁹, R¹⁰, R¹¹, R¹², R¹³, R¹⁴, if present, are independently selected from H, deuterium, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein the methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more deuterium, halogen, hydroxy, oxo, methanoyl (—COH), carboxy (—CO₂H), nitro (—NO₂), —NH₂, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CON(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl, provided that at least one of R⁷, R⁸, R⁹, R¹⁰, R¹¹, R¹², R¹³, and R¹⁴ is not H. In another embodiment, each of R⁷, R⁸, R⁹, R¹⁰, R¹¹, R¹², R¹³, and R¹⁴, if present, is H. In some embodiments, R¹⁵, R¹⁶, R¹⁷, R¹⁸, R¹⁹, R²⁰, R²¹, R²², R²³, R²⁴, R²⁵, R²⁶, R²⁷, R²⁹, R²⁹, and R³⁰, if present, are independently selected from H, deuterium, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein the methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more deuterium, halogen, hydroxy, oxo, methanoyl (—COH), carboxy (—CO₂H), nitro (—NO₂), —NH₂, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CON(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl. In some embodiments, m, n, o, p, q, r, s, t, u, v, w, and x are independently selected from 0, 1, 2, 3, 4, or 5, where q+r+s+t is at least 1, and where u+v+w+x is at least 1.

In an embodiment, each of R₃₁ and R₃₂ is independently selected from H, deuterium, C₁-C₆ alkyl, and C₃-C₆ cycloalkyl, wherein C₁-C₆ alkyl and C₃-C₆ cycloalkyl are each optionally substituted with one or more halogen.

In one embodiment, the compound of Formula (I) is a compound of Formula (I-5010), (II-5010), or (III-5010)

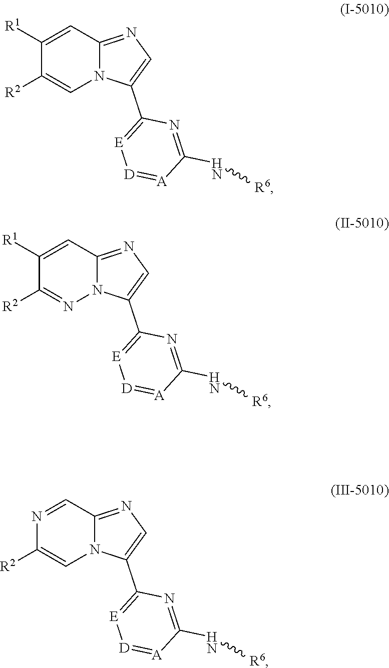

or a salt, ester, solvate, optical isomer, geometric isomer, salt of an isomer, prodrug, or derivative thereof, wherein A, D, E, R¹, R², and R⁶ are as defined in Formula (I), (II), and (III).

In one embodiment, the Formula (I), (II), or (III) is a compound of Formula (V), (VI), or (VII):

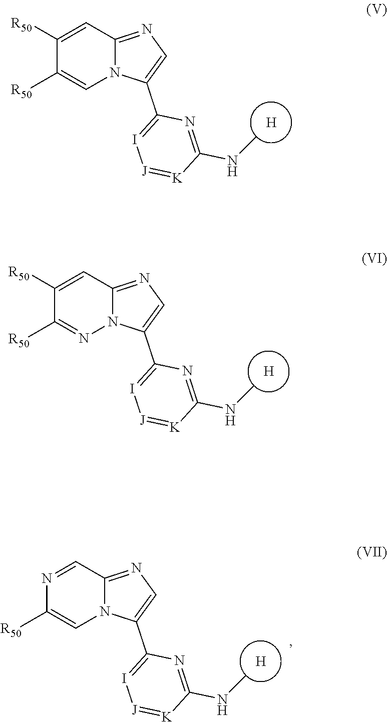

or a salt, ester, solvate, optical isomer, geometric isomer, salt of an isomer, prodrug, or derivative thereof;
wherein:
   I is N or CR₅₁;
   J is N or CR₅₂;
   K is N or CR₅₃;

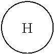

is selected from

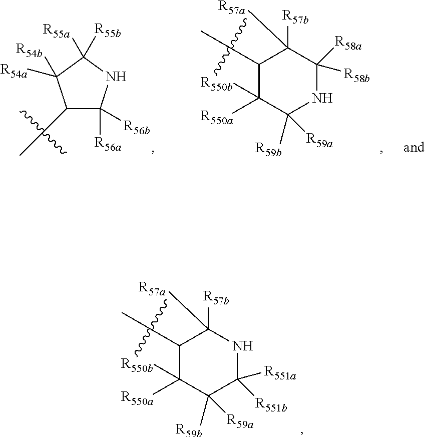

each R₅₀ is independently selected from H, halogen, C₁-C₆ alkyl, C₁-C₆ alkoxy, C₃-C₆ cycloalkyl, spiro-fused cycloalkyl, —O—(C₃-C₆ cycloalkyl), C₃-C₉ heteroaryl, C₂-C₉ heterocyclyl, and —C(═O)NR₅₅₂ₐR_{552b}, wherein C₁-C₆ alkyl and C₁-C₆ alkoxy are each optionally substituted with one or more substituents selected from —OH, deuterium, and halogen, and C₃-C₆ cycloalkyl, spiro-fused cycloalkyl, —O—(C₃-C₆ cycloalkyl), and C₂-C₉ heterocyclyl are each optionally substituted with one or more substituents selected from —OH, C₁-C₆ alkyl, and halogen;
R₅₁, R₅₂, and R₅₃ are each independently selected from H, C₁-C₆ alkoxy, and halogen;
R₅₄ₐ, R_{54b}, R₅₅ₐ, R_{55b}, R₅₆ₐ, R_{56b}, R₅₇ₐ, R_{57b}, R₅₈ₐ, R_{58b}, R₅₉ₐ, R_{59b}, R₅₅₀ₐ, R_{550b}, R₅₅₁ₐ, and R_{551b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms;
R₅₅₂ₐ and R_{552b} are each independently selected from H, C₁-C₆ alkyl, and C₃-C₆ cycloalkyl, wherein C₁-C₆ alkyl and C₃-C₆ cycloalkyl are each optionally substituted with one or more halogen; and
one of I, J, or K is N.

In one embodiment, wherein one or more of R₅₄ₐ, R_{54b}, R₅₅ₐ, R_{55b}, R₅₆ₐ, R_{56b}, R₅₇ₐ, R_{57b}, R₅₈ₐ, R_{58b}, R₅₉ₐ, R_{59b}, R₅₅₀ₐ, R_{550b}, R₅₅₁ₐ, and R_{551b} is selected from halogen, —OH, optionally substituted C₁-C₆ alkyl, and optionally substituted C₁-C₆ alkoxy. In another embodiment, each of R₅₄ₐ, R_{54b}, R₅₅ₐ, R_{55b}, R₅₆ₐ, R_{56b}, R₅₇ₐ, R_{57b}, R₅₈ₐ, R_{58b}, R₅₉ₐ, R_{59b}, R₅₅₀ₐ, R_{550b}, R₅₅₁ₐ, and R_{551b} is H.

### Formula (Ia-5010-WO)

In one embodiment, the compound of Formula (I) is a compound of Formula (Ia-5010-WO):

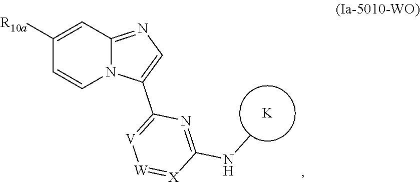

or a salt, ester, solvate, optical isomer, geometric isomer, or salt of an isomer thereof;
wherein:
   V is N or CR₁₁;
   W is N or CR₁₂;
   X is N or CR₁₃;

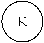

is

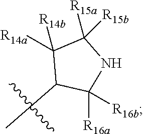

R₁₀ₐ is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₁₁, R₁₂, and R₁₃ are each independently selected from H, C₁-C₆ alkoxy, and halogen;
R₁₄ₐ, R_{14b}, R₁₅ₐ, R_{15b}, R₁₆ₐ, and R_{16b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms;
R₁₈ₐ and R_{18b} are each independently selected from H, C₁-C₆ alkyl, and C₃-C₆ cycloalkyl, wherein C₁-C₆ alkyl and C₃-C₆ cycloalkyl are each optionally substituted with one or more halogen; and
one of V, W, or X is N.

In an embodiment, one or more of R₁₄ₐ, R_{14b}, R₁₅ₐ, R_{15b}, R₁₆ₐ, and R_{16b} in Formula (Ia-5010-WO) is selected from halogen, —OH, optionally substituted C₁-C₆ alkyl, and optionally substituted C₁-C₆ alkoxy. In one embodiment, each of R₁₅ₐ, R_{15b}, R₁₆ₐ, and R_{16b} is H and R₁₄ₐ and/or R_{14b} is halogen. In one embodiment, each of R₁₅ₐ, R_{15b}, R₁₆ₐ, and R_{16b} is H and R₁₄ₐ and/or R_{14b} is F. In one embodiment, each of R_{14b}, R₁₅ₐ, R_{15b}, R₁₆ₐ, and R_{16b} is H and R₁₄ₐ is F.

In an embodiment, R₁₀ₐ in Formula (Ia-5010-WO) is unsubstituted C₁-C₆ alkoxy. In one embodiment, R₁₀ₐ is selected from —OCH₃ and

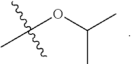 [SMILES: *OC(C)C]

In an embodiment, R₁₀ₐ is unsubstituted —O—(C₃-C₆ cycloalkyl). In one embodiment, R₁₀ₐ is unsubstituted —O—(C₃ cycloalkyl). In another embodiment, R₁₀ₐ is C₁-C₆ alkoxy substituted with one or more halogen. In one embodiment, R₁₀ₐ is selected from

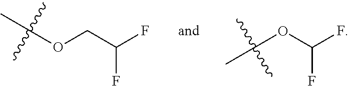 [SMILES: *OCC(F)F ; *OC(F)F]

In one embodiment, R₁₁, R₁₂, and R₁₃ of Formula (Ia-5010-WO), if present, are H.

In one embodiment, the compound of Formula (Ia-5010-WO) is a compound covered by the above formula and shown in Table 1 of the present application.

### Formula (Ib-5010-WO)

In another embodiment, the compound of Formula (I) is a compound of Formula (Ib-5010-WO):

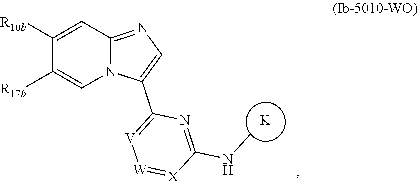

or a salt, ester, solvate, optical isomer, geometric isomer, or salt of an isomer thereof;
wherein:
   V is N or CR₁₁;
   W is N or CR₁₂;
   X is N or CR₁₃;

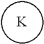

or

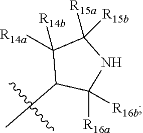

R_{10b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R_{17b} is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₁₁, R₁₂, and R₁₃ are each independently selected from H and halogen;
R₁₄ₐ, R_{14b}, R₁₅ₐ, R_{15b}, R₁₆ₐ, R_{16b}, R₁₈ₐ, and R_{18b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of V, W, or X is N.

In an embodiment, one or more of R₁₄ₐ, R_{14b}, R₁₅ₐ, R_{15b}, R₁₆ₐ, and R_{16b} in Formula (Ib-5010-WO) is selected from halogen, —OH, optionally substituted C₁-C₆ alkyl, and optionally substituted C₁-C₆ alkoxy. In one embodiment, each of R₁₅ₐ, R_{15b}, R₁₆ₐ, and R_{16b} is H and R₁₄ₐ and/or R_{14b} is halogen. In one embodiment, each of R₁₅ₐ, R_{15b}, R₁₆ₐ, and R_{16b} is H and R₁₄ₐ and/or R_{14b} is F. In one embodiment, each of R_{14b}, R₁₅ₐ, R_{15b}, R₁₆ₐ, and R_{16b} is H and R₁₄ₐ is F.

In an embodiment, R_{10b} in Formula (Ib-5010-WO) is H. In another embodiment, R_{10b} is unsubstituted C₁-C₆ alkoxy. In one embodiment, R_{10b} is —OCH₃.

In an embodiment, R_{17b} in Formula (Ib-5010-WO) is C₁-C₆ alkyl substituted with one —OH and/or halogen. In one embodiment, R_{17b} is selected from

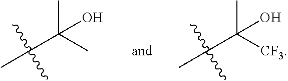

In one embodiment, the compound of Formula (Ib-5010-WO) is a compound covered by the above formula and shown in Table 1 of the present application.

### Formula (Ib-5010-WO50)

In an embodiment, the compound of Formula (I) is a compound of Formula (Ib-5010-WO50):

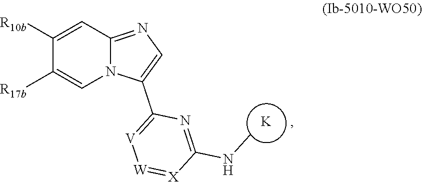

or a salt, ester, solvate, optical isomer, geometric isomer, or salt of an isomer thereof;
wherein:
   V is N or CR₁₁;
   W is N or CR₁₂;
   X is N or CR₁₃;

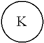

or

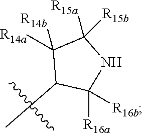

R_{10b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from C₁-C₆ alkyl and halogen;
R_{17b} is selected from halogen, C₁-C₆ alkyl, C₁-C₆ alkoxy, C₃-C₆ cycloalkyl, —O—(C₃-C₆ cycloalkyl), C₃-C₉ heterocyclyl, imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from —OH, C₁-C₆ alkyl, and halogen;
R₁₁, R₁₂, and R₁₃ are each independently selected from H, C₁-C₆ alkoxy, and halogen;
R₁₄ₐ, R_{14b}, R₁₅ₐ, R_{15b}, R₁₆ₐ, R_{16b}, R₁₈ₐ, and R_{18b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of V, W, or X is N.

In an embodiment, each of R₁₄ₐ, R_{14b}, R₁₅ₐ, R_{15b}, R₁₆ₐ, and R_{16b} in Formula (Ib-5010-WO50) is H. In an embodiment, one or more of R₁₄ₐ, R_{14b}, R₁₅ₐ, R_{15b}, R₁₆ₐ, and R_{16b} is selected from halogen, —OH, optionally substituted C₁-C₆ alkyl, and optionally substituted C₁-C₆ alkoxy. In one embodiment, each of R₁₅ₐ, R_{15b}, R₁₆ₐ, and R_{16b} is H and R₁₄ₐ and/or R_{14b} is halogen. In one embodiment, each of R₁₅ₐ, R_{15b}, R₁₆ₐ, and R_{16b} is H and R₁₄ₐ and/or R_{14b} is F. In one embodiment, each of R_{14b}, R₁₅ₐ, R_{15b}, R₁₆ₐ, and R_{16b} is H and R₁₄ₐ is F.

In an embodiment, R_{10b} in Formula (Ib-5010-WO50) is H. In another embodiment, R_{10b} is unsubstituted C₁-C₆ alkoxy. In one embodiment, R_{10b} is —OCH₃.

In one embodiment, R₁₁, R₁₂, and R₁₃, if present in Formula (Ib-5010-WO50), are H. In another embodiment, V is CR₁₁ wherein R₁₁ is F, W is CR₁₂ wherein R₁₂ is H, and X is N.

In an embodiment, R_{17b} in Formula (Ib-5010-WO50) is C₁-C₆ alkyl substituted with one —OH and/or halogen. In one embodiment, R_{17b} is selected from

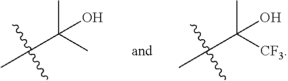

In one embodiment, the compound of Formula (I) is a compound of Formula (Ib-5010-WO50) with the proviso that when R_{17b} is

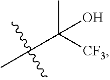 [SMILES: *C(C)(O)C(F)(F)F]

R_{10b} is H. In one embodiment, compound of Formula (I) is a compound of Formula (Ib-5010-WO50) with the proviso that when R_{17b} is

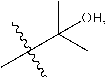 [SMILES: *C(C)(C)O]

at least one of R₁₁, R₁₂, and R₁₃ is present and selected from C₁-C₆ alkoxy and halogen.

In one embodiment, the compound of Formula (Ib-5010-WO50) is not:

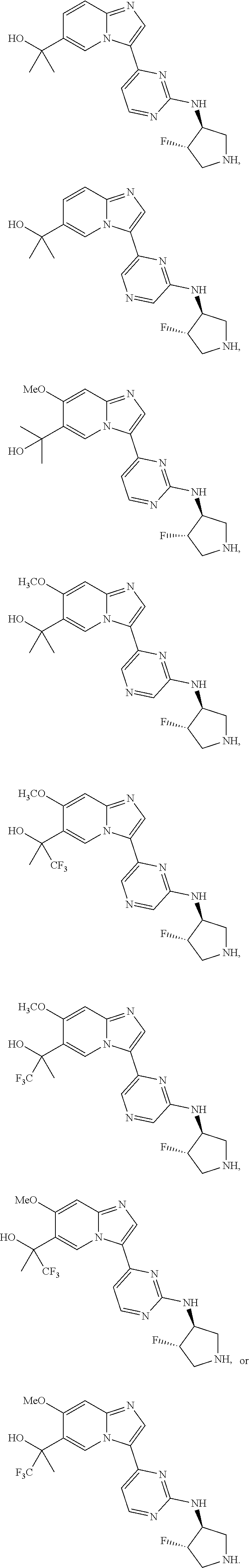

In an embodiment, the compound of Formula (Ib-5010-WO50) is selected from:

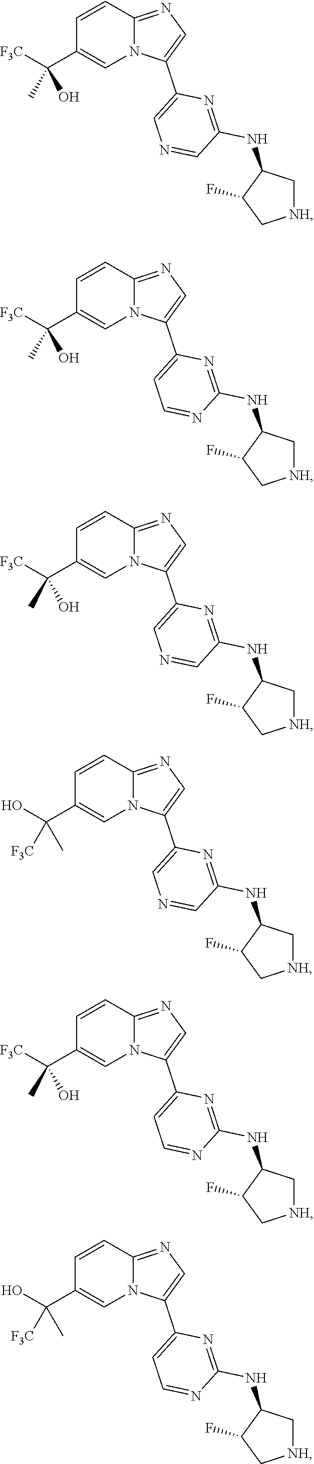 [SMILES: C[C@@](O)(c1ccc2ncc(-c3cncc(N[C@H]4CNC[C@@H]4F)n3)n2c1)C(F)(F)F ; C[C@@](O)(c1ccc2ncc(-c3ccnc(N[C@H]4CNC[C@@H]4F)n3)n2c1)C(F)(F)F ; C[C@](O)(c1ccc2ncc(-c3cncc(N[C@H]4CNC[C@@H]4F)n3)n2c1)C(F)(F)F ; CC(O)(c1ccc2ncc(-c3cncc(N[C@H]4CNC[C@@H]4F)n3)n2c1)C(F)(F)F ; C[C@](O)(c1ccc2ncc(-c3ccnc(N[C@H]4CNC[C@@H]4F)n3)n2c1)C(F)(F)F ; CC(O)(c1ccc2ncc(-c3ccnc(N[C@H]4CNC[C@@H]4F)n3)n2c1)C(F)(F)F]

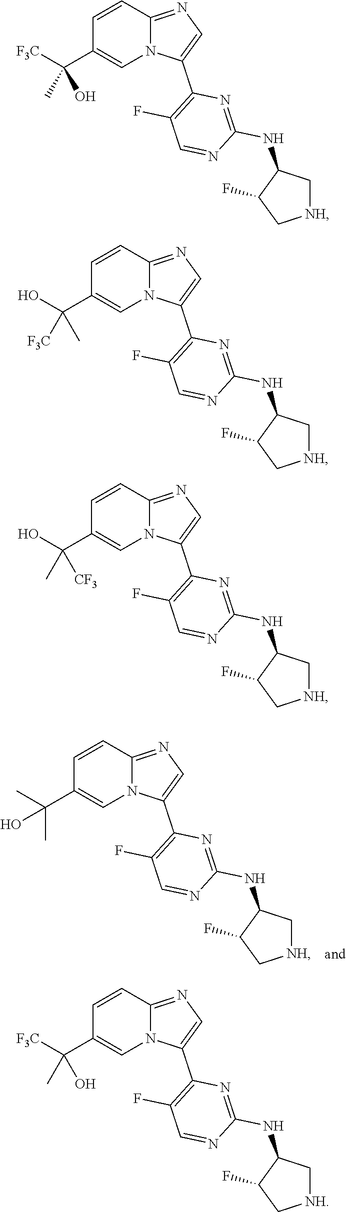 [SMILES: C[C@@](O)(c1ccc2ncc(-c3nc(N[C@H]4CNC[C@@H]4F)ncc3F)n2c1)C(F)(F)F ; CC(O)(c1ccc2ncc(-c3nc(N[C@H]4CNC[C@@H]4F)ncc3F)n2c1)C(F)(F)F ; CC(O)(c1ccc2ncc(-c3nc(N[C@H]4CNC[C@@H]4F)ncc3F)n2c1)C(F)(F)F ; CC(C)(O)c1ccc2ncc(-c3nc(N[C@H]4CNC[C@@H]4F)ncc3F)n2c1 ; CC(O)(c1ccc2ncc(-c3nc(N[C@H]4CNC[C@@H]4F)ncc3F)n2c1)C(F)(F)F]

In an embodiment, the compound of Formula (Ib-5010-WO50) is selected from:

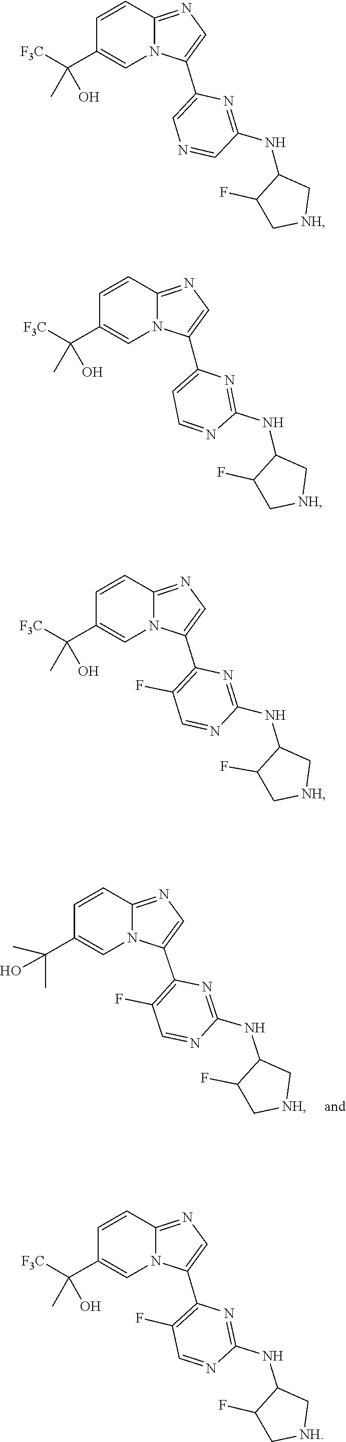 [SMILES: CC(O)(c1ccc2ncc(-c3cncc(NC4CNCC4F)n3)n2c1)C(F)(F)F ; CC(O)(c1ccc2ncc(-c3ccnc(NC4CNCC4F)n3)n2c1)C(F)(F)F ; CC(O)(c1ccc2ncc(-c3nc(NC4CNCC4F)ncc3F)n2c1)C(F)(F)F ; CC(C)(O)c1ccc2ncc(-c3nc(NC4CNCC4F)ncc3F)n2c1 ; CC(O)(c1ccc2ncc(-c3nc(NC4CNCC4F)ncc3F)n2c1)C(F)(F)F]

### Formula (Ic-5010-WO)

In another embodiment, the compound of Formula (I) is a compound of Formula (Ic-5010-WO):

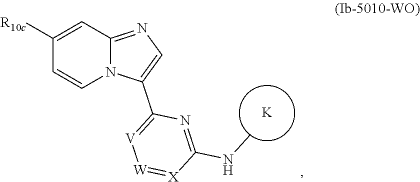

or a salt, ester, solvate, optical isomer, geometric isomer, or salt of an isomer thereof;
wherein:
   V is N or CR₁₁;
   W is N or CR₁₂;
   X is N or CR₁₃;

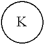

is

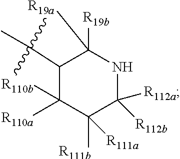

R_{10c} is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₁₁, R₁₂, and R₁₃ are each independently selected from H, C₁-C₆ alkoxy, and halogen;
R₁₉ₐ, R_{19b}, R₁₁₀ₐ, R_{110b}, R₁₁₁ₐ, R_{111b}, R₁₁₂ₐ, and R_{112b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms;
R₁₈ₐ and R_{18b} are each independently selected from H, C₁-C₆ alkyl, and C₃-C₆ cycloalkyl, wherein C₁-C₆ alkyl and C₃-C₆ cycloalkyl are each optionally substituted with one or more halogen; and
one of V, W, or X is N.

In an embodiment, each of R₁₉ₐ, R_{19b}, R₁₁₀ₐ, R_{110b}, R₁₁₁ₐ, R_{111b}, R₁₁₂ₐ, and R_{112b} of Formula (Ic-5010-WO) is H. In another embodiment, one or more R₁₉ₐ, R_{19b}, R₁₁₀ₐ, R_{110b}, R₁₁₁ₐ, R_{111b}, R₁₁₂ₐ, and R_{112b} is selected from halogen, —OH, optionally substituted C₁-C₆ alkyl, and optionally substituted C₁-C₆ alkoxy. In one embodiment, each of R₁₉ₐ, R_{19b}, R₁₁₁ₐ, R_{111b}, R₁₁₂ₐ, and R_{112b} is H and R₁₁₀ₐ and/or R_{110b} is halogen. In one embodiment, each of R₁₉ₐ, R_{19b}, R₁₁₁ₐ, R_{111b}, R₁₁₂ₐ, and R_{112b} is H and R₁₁₀ₐ and/or R_{110b} is F. In one embodiment, R₁₉ₐ, R_{19b}, R_{110b}, R₁₁₁ₐ, R_{111b}, R₁₁₂ₐ, and R_{112b} is H and R₁₁₀ₐ is F.

In an embodiment, R_{10c} in Formula (Ic-5010-WO) is unsubstituted C₁-C₆ alkoxy. In one embodiment, R_{10c} is selected from —OCH₃ and

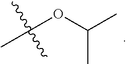 [SMILES: *OC(C)C]

In an embodiment, R_{10c} is unsubstituted —O—(C₃-C₆ cycloalkyl). In one embodiment, R_{10c} is unsubstituted —O—(C₃ cycloalkyl). In another embodiment, R_{10c} is C₁-C₆ alkoxy substituted with one or more halogen.

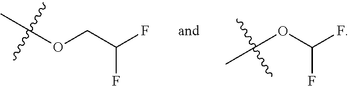 [SMILES: *OCC(F)F ; *OC(F)F]

In one embodiment, R_{10c} is selected from F and F

In one embodiment, R₁₁, R₁₂, and R₁₃ in Formula (Ic-5010-WO), if present, are H.

In an embodiment, the compound of Formula (Ic-5010-WO) is a compound covered by the above formula and shown in Table 1 of the present application.

### Formula (Id-5010-WO)

In another embodiment, the compound of Formula (I) is a compound of Formula (Id-5010-WO):

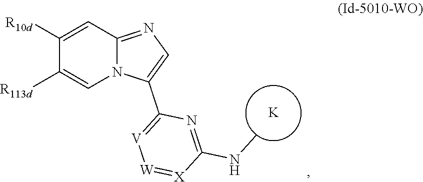

or a salt, ester, solvate, optical isomer, geometric isomer, or salt of an isomer thereof;
wherein:
   V is N or CR₁₁;
   W is N or CR₁₂;
   X is N or CR₁₃;

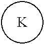

is

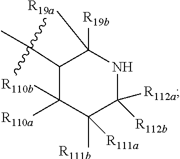

R_{10d} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R_{113d} is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and wherein C₃-C₆ cycloalkyl and —O—(C₃-C₆ cycloalkyl) are each optionally substituted with one or more substituents selected from C₁-C₆ alkyl and halogen;
R₁₁, R₁₂, and R₁₃ are each independently selected from H and halogen;
R₁₈ₐ, R_{18b}, R₁₉ₐ, R_{19b}, R₁₁₀ₐ, R_{110b}, R₁₁₁ₐ, R_{111b}, R₁₁₂ₐ, and R_{112b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of V, W, or X is N.

In an embodiment, each of R₁₉ₐ, R_{19b}, R₁₁₀ₐ, R_{110b}, R₁₁₁ₐ, R_{111b}, R₁₁₂ₐ, and R_{112b} in Formula (Id-5010-WO) is H. In another embodiment, one or more R₁₉ₐ, R_{19b}, R₁₁₀ₐ, R_{110b}, R₁₁₁ₐ, R_{111b}, R₁₁₂ₐ, and R_{112b} is selected from halogen, —OH, optionally substituted C₁-C₆ alkyl, and optionally substituted C₁-C₆ alkoxy. In one embodiment, each of R₁₉ₐ, R_{19b}, R₁₁₁ₐ, R_{111b}, R₁₁₂ₐ, and R_{112b} is H and R₁₁₀ₐ and/or R_{110b} is halogen. In one embodiment, each of R₁₉ₐ, R_{19b}, R₁₁₁ₐ, R_{111b}, R₁₁₂ₐ, and R_{112b} is H and R₁₁₀ₐ and/or R_{110b} is F. In one embodiment, R₁₉ₐ, R_{19b}, R_{110b}, R₁₁₁ₐ, R_{111b}, R₁₁₂ₐ, and R_{112b} is H and R₁₁₀ₐ is F.

In an embodiment, R_{10d} in Formula (Id-5010-WO) is H. In another embodiment, R_{10d} is unsubstituted C₁-C₆ alkoxy. In one embodiment, R_{10d} is —OCH₃.

In an embodiment, R_{113d} of Formula (Id-5010-WO) is C₁-C₆ alkyl substituted with one —OH and/or halogen. In one embodiment, R_{113d} is selected from

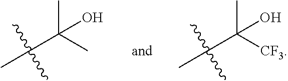

In an embodiment, the compound of Formula (Id-5010-WO) is a compound covered by the above formula and shown in Table 1 of the present application.

### Formula (Id-5010-WO50)

In another embodiment, the compound of Formula (I) is a compound of Formula (Id-5010-WO50):

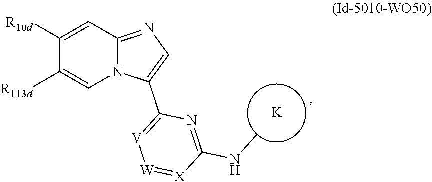

or a salt, ester, solvate, optical isomer, geometric isomer, or salt of an isomer thereof;
wherein:
   V is N or CR₁₁;
   W is N or CR₁₂;
   X is N or CR₁₃;

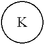

is

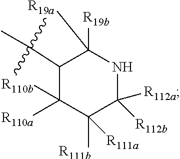

R_{10d} is selected from H, halogen, C₁-C₆ alkyl, C₁-C₆alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and wherein halogen, and C₃-C₆ cycloalkyl and —O—(C₃-C₆ cycloalkyl) are each optionally substituted with one or more substituents selected from C₁-C₆ alkyl and halogen;
R_{113d} is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from —OH, C₁-C₆ alkyl, and halogen,
R₁₁, R₁₂, and R₁₃ are each independently selected from H, C₁-C₆ alkoxy, and halogen; R₁₈ₐ and R_{18b} are each independently selected from H, C₁-C₆ alkyl, and C₃-C₆ cycloalkyl, wherein C₁-C₆ alkyl and C₃-C₆ cycloalkyl are each optionally substituted with one or more halogen;
R₁₉ₐ, R_{19b}, R₁₁₀ₐ, R_{110b}, R₁₁₁ₐ, R_{111b}, R₁₁₂ₐ, and R_{112b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of V, W, or X is N.

In an embodiment, each of Risa, R_{19b}, R₁₁₀ₐ, R_{110b}, R₁₁₁ₐ, R_{111b}, R₁₁₂ₐ, and R_{112b} of Formula (Id-5010-WO50) is H. In another embodiment, one or more R₁₉ₐ, R_{19b}, R₁₁₀ₐ, R_{110b}, R₁₁₁ₐ, R_{111b}, R₁₁₂ₐ, and R_{112b} is selected from halogen, —OH, optionally substituted C₁-C₆ alkyl, and optionally substituted C₁-C₆ alkoxy. In one embodiment, each of R₁₉ₐ, R_{19b}, R₁₁₁ₐ, R_{111b}, R₁₁₂ₐ, and R_{112b} is H and R₁₁₀ₐ and/or R_{110b} is halogen. In one embodiment, each of R₁₉ₐ, R_{19b}, R₁₁₁ₐ, R_{111b}, R₁₁₂ₐ, and R_{112b} is H and R₁₁₀ₐ and/or R_{110b} is F. In one embodiment, R₁₉ₐ, R_{19b}, R_{110b}, R₁₁₁ₐ, R_{111b}, R₁₁₂ₐ, and R_{112b} is H and R₁₁₀ₐ is F.

In an embodiment, R_{10d} of Formula (Id-5010-WO50) is H. In another embodiment, R_{10d} is unsubstituted C₁-C₆ alkoxy. In one embodiment, R_{10d} is —OCH₃.

In one embodiment, R₁₁, R₁₂, and R₁₃ of Formula (Id-5010-WO50), if present, are H. In another embodiment, V is CR₁₁ wherein R₁₁ is F, W is CR₁₂ wherein R₁₂ is H, and X is N.

In an embodiment, R_{113d} of Formula (Id-5010-WO50) is C₁-C₆ alkyl substituted with one —OH and/or halogen. In one embodiment, R_{113d} is selected from

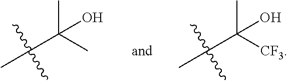

In one embodiment, the compound of Formula (I) is a compound of Formula (Id-5010-WO50) with the proviso that when R_{113d} is

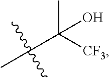 [SMILES: *C(C)(O)C(F)(F)F]

R_{10d} is H. In one embodiment, the compound of Formula (I) is a compound of Formula (Id-5010-WO50) with the proviso that when R_{113d} is

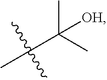 [SMILES: *C(C)(C)O]

at least one of R₁₁, R₁₂, and R₁₃ is present and selected from C₁-C₆ alkoxy and halogen.

In one embodiment, the compound of Formula (Id-5010-WO50) is not:

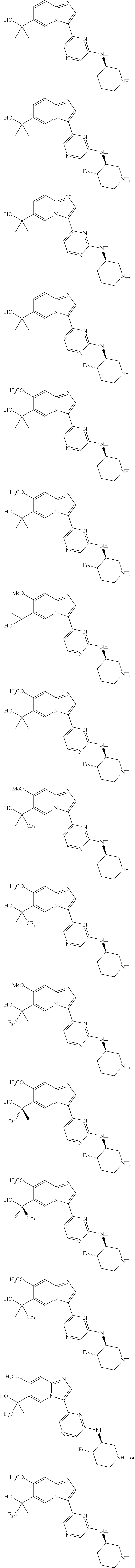

In an embodiment, the compound of Formula (Id-5010-WO50) is selected from:

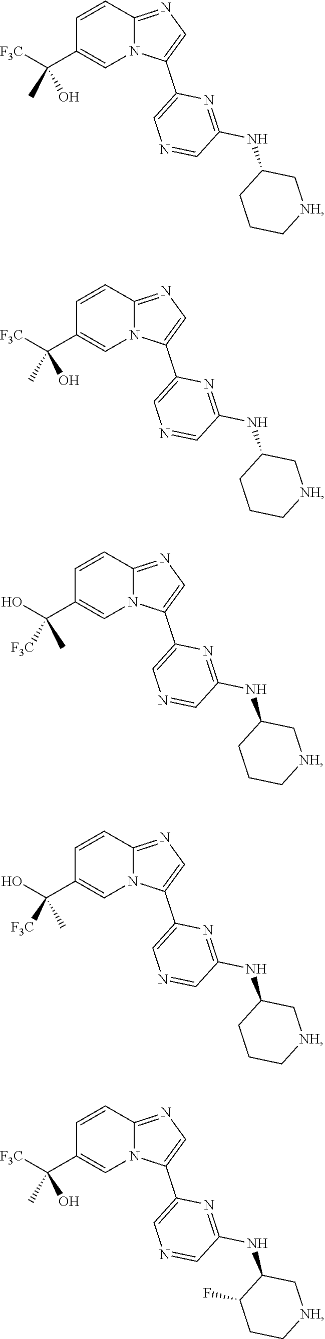 [SMILES: C[C@](O)(c1ccc2ncc(-c3cncc(N[C@H]4CCCNC4)n3)n2c1)C(F)(F)F ; C[C@@](O)(c1ccc2ncc(-c3cncc(N[C@H]4CCCNC4)n3)n2c1)C(F)(F)F ; C[C@@](O)(c1ccc2ncc(-c3cncc(N[C@@H]4CCCNC4)n3)n2c1)C(F)(F)F ; C[C@](O)(c1ccc2ncc(-c3cncc(N[C@@H]4CCCNC4)n3)n2c1)C(F)(F)F ; C[C@@](O)(c1ccc2ncc(-c3cncc(N[C@H]4CNCC[C@@H]4F)n3)n2c1)C(F)(F)F]

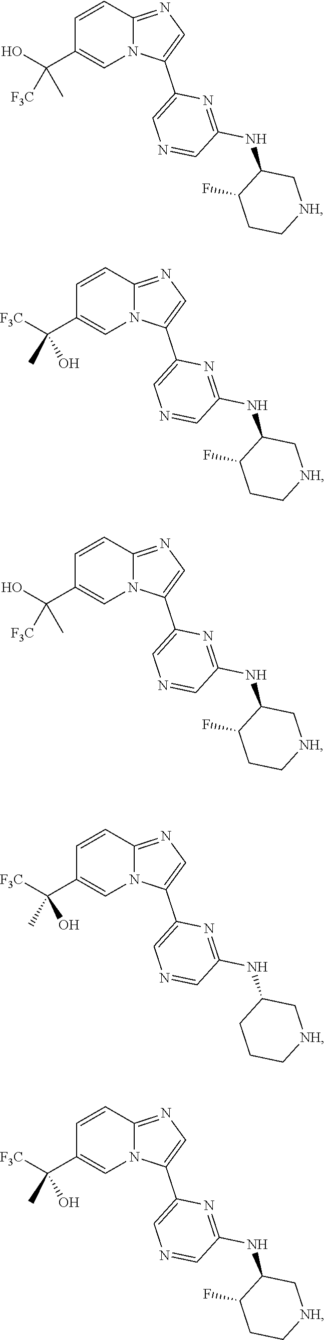 [SMILES: CC(O)(c1ccc2ncc(-c3cncc(N[C@H]4CNCC[C@@H]4F)n3)n2c1)C(F)(F)F ; C[C@](O)(c1ccc2ncc(-c3cncc(N[C@H]4CNCC[C@@H]4F)n3)n2c1)C(F)(F)F ; CC(O)(c1ccc2ncc(-c3cncc(N[C@H]4CNCC[C@@H]4F)n3)n2c1)C(F)(F)F ; C[C@@](O)(c1ccc2ncc(-c3cncc(N[C@H]4CCCNC4)n3)n2c1)C(F)(F)F ; C[C@](O)(c1ccc2ncc(-c3cncc(N[C@H]4CNCC[C@@H]4F)n3)n2c1)C(F)(F)F]

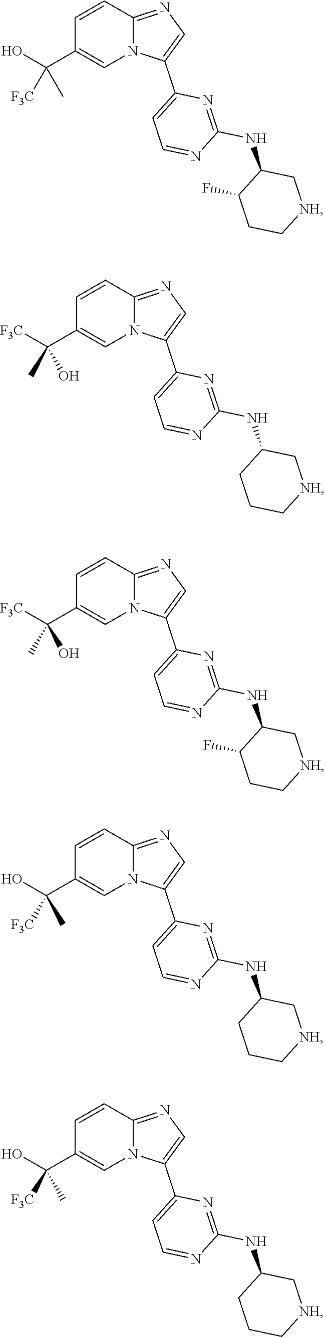 [SMILES: CC(O)(c1ccc2ncc(-c3ccnc(N[C@H]4CNCC[C@@H]4F)n3)n2c1)C(F)(F)F ; C[C@](O)(c1ccc2ncc(-c3ccnc(N[C@H]4CCCNC4)n3)n2c1)C(F)(F)F ; C[C@@](O)(c1ccc2ncc(-c3ccnc(N[C@H]4CNCC[C@@H]4F)n3)n2c1)C(F)(F)F ; C[C@@](O)(c1ccc2ncc(-c3ccnc(N[C@@H]4CCCNC4)n3)n2c1)C(F)(F)F ; C[C@](O)(c1ccc2ncc(-c3ccnc(N[C@@H]4CCCNC4)n3)n2c1)C(F)(F)F]

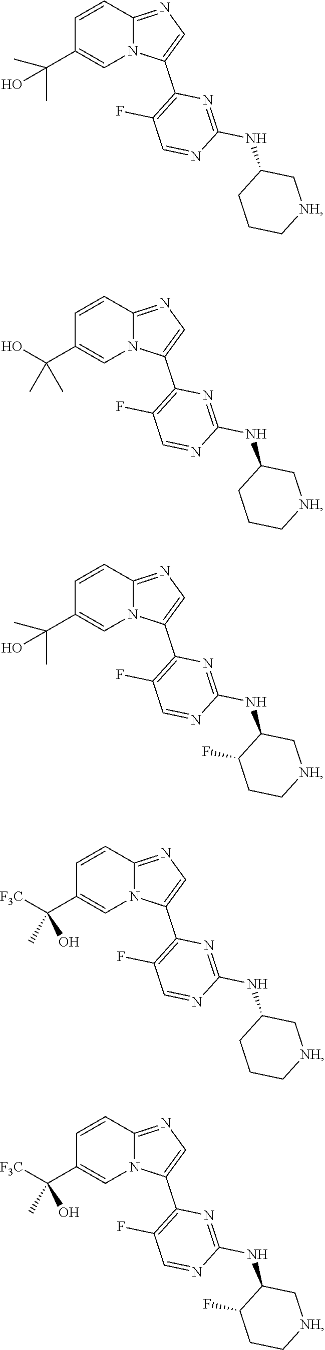 [SMILES: CC(C)(O)c1ccc2ncc(-c3nc(N[C@H]4CCCNC4)ncc3F)n2c1 ; CC(C)(O)c1ccc2ncc(-c3nc(N[C@@H]4CCCNC4)ncc3F)n2c1 ; CC(C)(O)c1ccc2ncc(-c3nc(N[C@H]4CNCC[C@@H]4F)ncc3F)n2c1 ; C[C@@](O)(c1ccc2ncc(-c3nc(N[C@H]4CCCNC4)ncc3F)n2c1)C(F)(F)F ; C[C@@](O)(c1ccc2ncc(-c3nc(N[C@H]4CNCC[C@@H]4F)ncc3F)n2c1)C(F)(F)F]

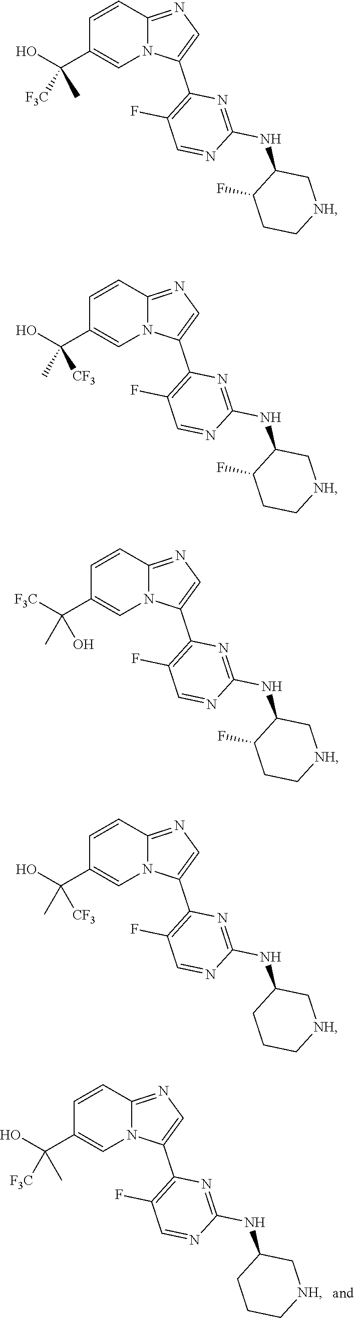 [SMILES: C[C@@](O)(c1ccc2ncc(-c3nc(N[C@H]4CNCC[C@@H]4F)ncc3F)n2c1)C(F)(F)F ; C[C@](O)(c1ccc2ncc(-c3nc(N[C@H]4CNCC[C@@H]4F)ncc3F)n2c1)C(F)(F)F ; CC(O)(c1ccc2ncc(-c3nc(N[C@H]4CNCC[C@@H]4F)ncc3F)n2c1)C(F)(F)F ; CC(O)(c1ccc2ncc(-c3nc(N[C@@H]4CCCNC4)ncc3F)n2c1)C(F)(F)F ; CC(O)(c1ccc2ncc(-c3nc(N[C@@H]4CCCNC4)ncc3F)n2c1)C(F)(F)F ; "and"]

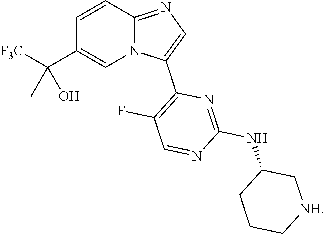 [SMILES: CC(O)(c1ccc2ncc(-c3nc(N[C@H]4CCCNC4)ncc3F)n2c1)C(F)(F)F]

In one embodiment, the compound of Formula (Id-5010-WO50) is selected from:

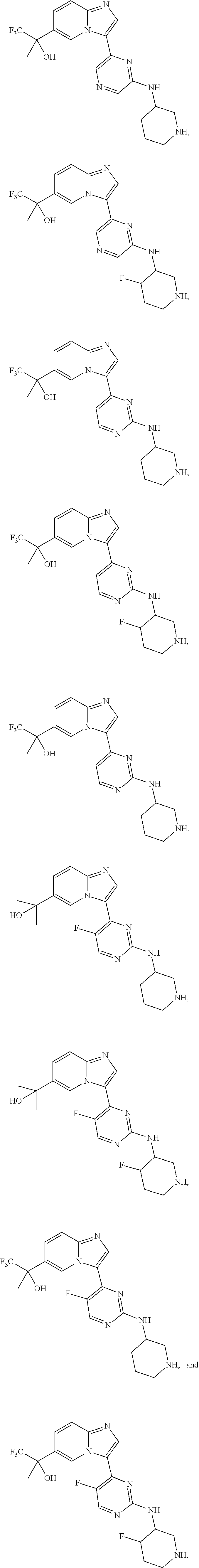

### Formula (IIa-5010-WO)

In one embodiment, the compound of Formula (II) is a compound of Formula (IIa-5010-WO):

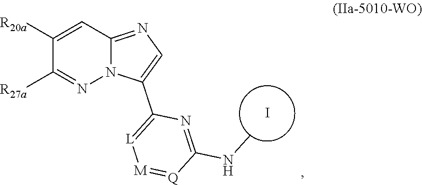

or a salt, ester, solvate, optical isomer, geometric isomer, or salt of an isomer thereof;
wherein:
   L is N or CR₂₁;
   M is N or CR₂₂;
   Q is N or CR₂₃;

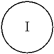

is

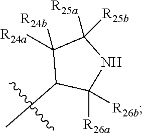

R₂₀ₐ is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₂₇ₐ is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₂₁, R₂₂, and R₂₃ are each independently selected from H and halogen;
R₂₄ₐ, R_{24b}, R₂₅ₐ, R_{25b}, R₂₆ₐ, R_{26b}, R₂₈ₐ, and R_{28b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of L, M, or Q is N.

In an embodiment, one or more of R₂₄ₐ, R_{24b}, R₂₅ₐ, R_{25b}, R₂₆ₐ, and R_{26b} of Formula (IIa-5010-WO) is selected from halogen, —OH, optionally substituted C₁-C₆ alkyl, and optionally substituted C₁-C₆ alkoxy. In one embodiment, each of R₂₅ₐ, R_{25b}, R₂₆ₐ, and R_{26b} is H and R₂₄ₐ and/or R_{24b} is halogen. In one embodiment, each of R₂₅ₐ, R_{25b}, R₂₆ₐ, and R_{26b} is H and R₂₄ₐ and/or R_{24b} is F. In one embodiment, each of R_{24b}, R₂₅ₐ, R_{25b}, R₂₆ₐ, and R_{26b} is H and R₂₄ₐ is F.

In an embodiment, R₂₀ₐ of Formula (IIa-5010-WO) is not H. In an embodiment, R₂₀ₐ is unsubstituted C₁-C₆ alkoxy. In one embodiment, R₂₀ₐ is —OCH₃.

In an embodiment, R₂₇ₐ of Formula (IIa-5010-WO) is unsubstituted C₃-C₆ cycloalkyl. In one embodiment, R₂₇ₐ is unsubstituted C₃ cycloalkyl. In an embodiment, R₂₇ₐ is C₁-C₆ alkyl substituted with one —OH and/or halogen. In one embodiment, R₂₇ₐ is

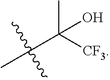 [SMILES: *C(C)(O)C(F)(F)F]

In an embodiment, the compound of Formula (IIa-5010-WO) is a compound covered by the above formula and shown in Table 1 of the present application.

### Formula (Ha-5010-WO50)

In one embodiment, the compound of Formula (II) is a compound of Formula (IIa-5010-WO50):

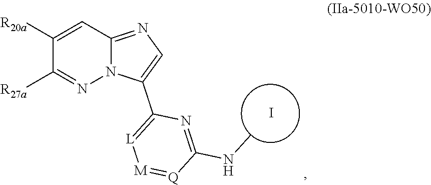

or a salt, ester, solvate, optical isomer, geometric isomer, or salt of an isomer thereof;
wherein:
   L is N or CR₂₁;
   M is N or CR₂₂;
   Q is N or CR₂₃;

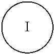

is

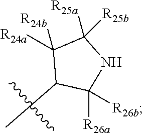

R₂₀ₐ is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from C₁-C₆ alkyl and halogen;
R₂₇ₐ is selected from halogen, C₁-C₆ alkyl, C₁-C₆ alkoxy, C₃-C₆ cycloalkyl, spiro-fused cycloalkyl, —O—(C₃-C₆ cycloalkyl), C₂-C₆ heterocyclyl, imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl, spiro-cycloalkyl, —O—(C₃-C₆ cycloalkyl), and C₂-C₆ heterocyclyl are each optionally substituted with one or more substituents selected from —OH, C₁-C₆ alkyl, and halogen;
R₂₁, R₂₂, and R₂₃ are each independently selected from H, C₁-C₆ alkoxy, and halogen;
R₂₄ₐ, R_{24b}, R₂₅ₐ, R_{25b}, R₂₆ₐ, and R_{26b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms;
R₂₈ₐ and R_{28b} are each independently selected from H, C₁-C₆ alkyl, and C₃-C₆ cycloalkyl, wherein C₁-C₆ alkyl and C₃-C₆ cycloalkyl are each optionally substituted with one or more halogen; and
one of L, M, or Q is N.

In an embodiment, one or more of R₂₄ₐ, R_{24b}, R₂₅ₐ, R_{25b}, R₂₆ₐ, and R_{26b} in Formula (IIa-5010-WO50) is selected from halogen, —OH, optionally substituted C₁-C₆ alkyl, and optionally substituted C₁-C₆ alkoxy. In one embodiment, each of R₂₅ₐ, R_{25b}, R₂₆ₐ, and R_{26b} is H and R₂₄ₐ and/or R_{24b} is halogen. In one embodiment, each of R₂₅ₐ, R_{25b}, R₂₆ₐ, and R_{26b} is H and R₂₄ₐ and/or R_{24b} is F. In one embodiment, each of R_{24b}, R₂₅ₐ, R_{25b}, R₂₆ₐ, and R_{26b} is H and R₂₄ₐ is F.

In an embodiment, R₂₀ₐ in Formula (IIa-5010-WO50) is not H. In an embodiment, R₂₀ₐ is unsubstituted C₁-C₆ alkoxy. In one embodiment, R₂₀ₐ is —OCH₃. In another embodiment, R₂₀ₐ is C₁-C₆ alkoxy substituted with one or more halogen. In one embodiment, R₂₀ₐ is

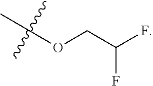 [SMILES: *OCC(F)F]

In another embodiment, R₂₀ₐ is unsubstituted —O—(C₃-C₆ cycloalkyl). In one embodiment, R₂₀ₐ is unsubstituted —O—C₃ cycloalkyl.

In one R₂₁, R₂₂, and R₂₃ in Formula (IIa-5010-WO50), if present, are H. In another embodiment, L is CR₂₁ wherein R₂₁ is selected from F, Cl, and —OCH₃, M is CR₂₂ wherein R₂₂ is H, and Q is N.

In an embodiment, R₂₇ₐ of Formula (IIa-5010-WO50) is unsubstituted C₃-C₆ cycloalkyl. In one embodiment, R₂₇ₐ is unsubstituted C₃ cycloalkyl or

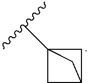 [SMILES: *C12CC(C1)C2]

In another embodiment, R₂₇ₐ is C₃-C₆ cycloalkyl substituted with one or more —CH₃ and/or F. In one embodiment, R₂₇ₐ is selected from

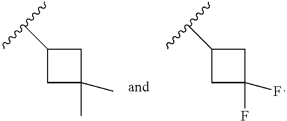

In an embodiment, R₂₇ₐ is C₁-C₆ alkyl substituted with one or more —OH and/or one or more halogen. In one embodiment, R₂₇ₐ is selected from

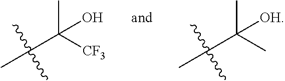

In one embodiment, R₂₇ₐ is spiro-fused cycloalkyl. In one embodiment, R₂₇ₐ is

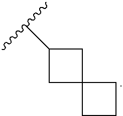 [SMILES: *C1CC2(CCC2)C1]

In another embodiment, R₂₇ₐ is unsubstituted C₂-C₆ heterocyclyl. In one embodiment, R₂₇ₐ is unsubstituted C₂-C₆ heterocyclyl comprising one oxygen atom, one nitrogen atom, or one oxygen atom and one nitrogen atom. In one embodiment, R₂₇ₐ is selected from

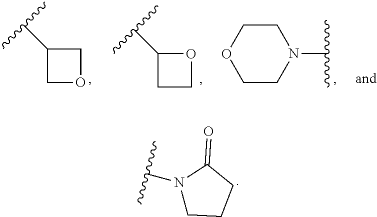

In one embodiment, R₂₇ₐ is

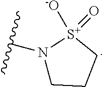

In one embodiment, R₂₇ₐ is

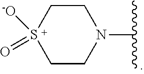

In another embodiment, R₂₇ₐ is a C₂-C₆ heterocyclyl substituted with one or more F. In one embodiment, R₂₇ₐ is

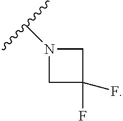 [SMILES: *N1CC(F)(F)C1]

In one embodiment, the compound of Formula (II) is a compound of Formula (IIa-5010-WO50) with the proviso that when R₂₀ₐ is —OCH₃ and R₂₇ₐ is unsubstituted C₃ cycloalkyl, at least one of R₂₁, R₂₂, and R₂₃ is present and selected from C₁-C₆ alkoxy and halogen.

In one embodiment, the compound of Formula (IIa-5010-WO50) is not:

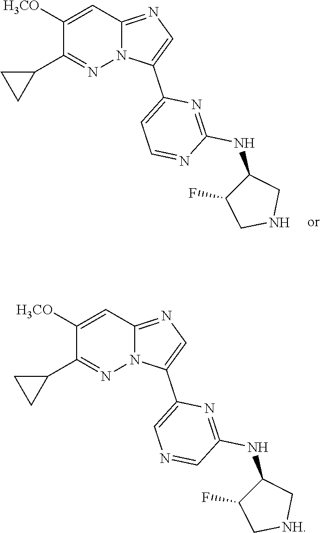 [SMILES: COc1cc2ncc(-c3ccnc(N[C@H]4CNC[C@@H]4F)n3)n2nc1C1CC1 ; "or" ; COc1cc2ncc(-c3cncc(N[C@H]4CNC[C@@H]4F)n3)n2nc1C1CC1]

In an embodiment, the compound of Formula (IIa-5010-WO50) is selected from:

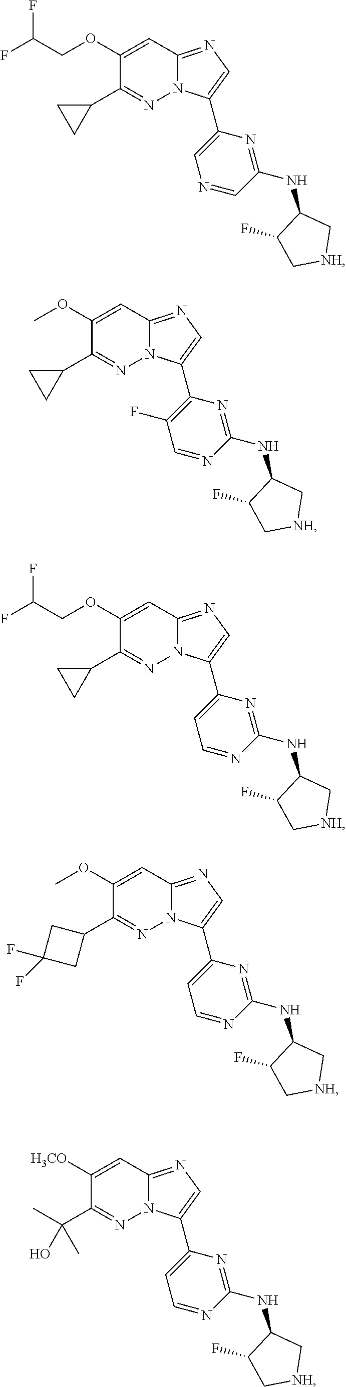 [SMILES: FC(F)COc1cc2ncc(-c3cncc(N[C@H]4CNC[C@@H]4F)n3)n2nc1C1CC1 ; COc1cc2ncc(-c3nc(N[C@H]4CNC[C@@H]4F)ncc3F)n2nc1C1CC1 ; FC(F)COc1cc2ncc(-c3ccnc(N[C@H]4CNC[C@@H]4F)n3)n2nc1C1CC1 ; COc1cc2ncc(-c3ccnc(N[C@H]4CNC[C@@H]4F)n3)n2nc1C1CC(F)(F)C1 ; COc1cc2ncc(-c3ccnc(N[C@H]4CNC[C@@H]4F)n3)n2nc1C(C)(C)O]

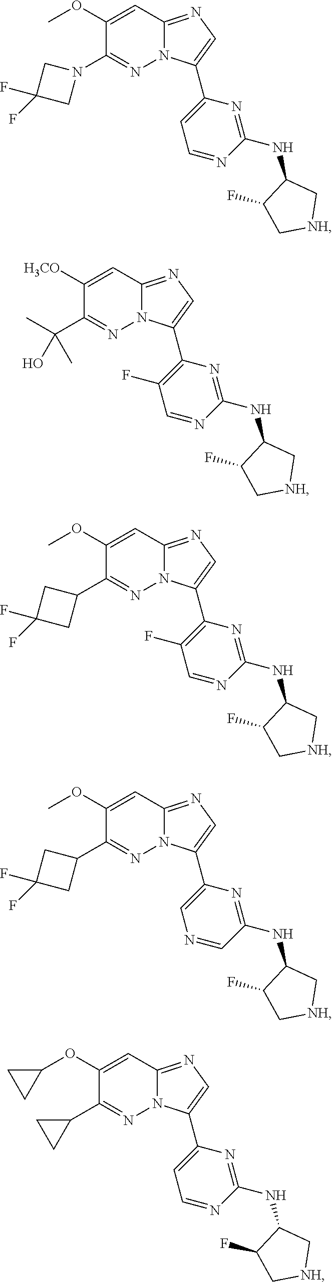 [SMILES: COc1cc2ncc(-c3ccnc(N[C@H]4CNC[C@@H]4F)n3)n2nc1N1CC(F)(F)C1 ; COc1cc2ncc(-c3nc(N[C@H]4CNC[C@@H]4F)ncc3F)n2nc1C(C)(C)O ; COc1cc2ncc(-c3nc(N[C@H]4CNC[C@@H]4F)ncc3F)n2nc1C1CC(F)(F)C1 ; COc1cc2ncc(-c3cncc(N[C@H]4CNC[C@@H]4F)n3)n2nc1C1CC(F)(F)C1 ; F[C@@H]1CNC[C@H]1Nc1nccc(-c2cnc3cc(OC4CC4)c(C4CC4)nn23)n1]

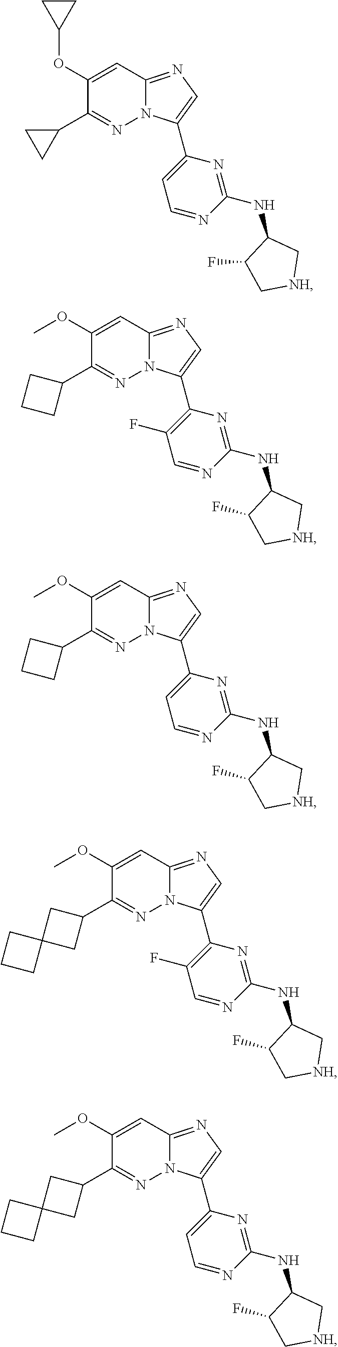 [SMILES: F[C@H]1CNC[C@@H]1Nc1nccc(-c2cnc3cc(OC4CC4)c(C4CC4)nn23)n1 ; COc1cc2ncc(-c3nc(N[C@H]4CNC[C@@H]4F)ncc3F)n2nc1C1CCC1 ; COc1cc2ncc(-c3ccnc(N[C@H]4CNC[C@@H]4F)n3)n2nc1C1CCC1 ; COc1cc2ncc(-c3nc(N[C@H]4CNC[C@@H]4F)ncc3F)n2nc1C1CC2(CCC2)C1 ; COc1cc2ncc(-c3ccnc(N[C@H]4CNC[C@@H]4F)n3)n2nc1C1CC2(CCC2)C1]

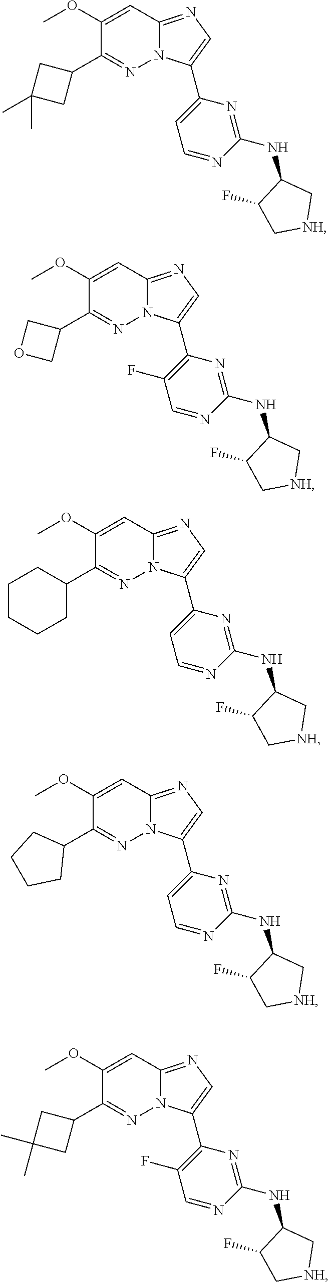 [SMILES: COc1cc2ncc(-c3ccnc(N[C@H]4CNC[C@@H]4F)n3)n2nc1C1CC(C)(C)C1 ; COc1cc2ncc(-c3nc(N[C@H]4CNC[C@@H]4F)ncc3F)n2nc1C1COC1 ; COc1cc2ncc(-c3ccnc(N[C@H]4CNC[C@@H]4F)n3)n2nc1C1CCCCC1 ; COc1cc2ncc(-c3ccnc(N[C@H]4CNC[C@@H]4F)n3)n2nc1C1CCCC1 ; COc1cc2ncc(-c3nc(N[C@H]4CNC[C@@H]4F)ncc3F)n2nc1C1CC(C)(C)C1]

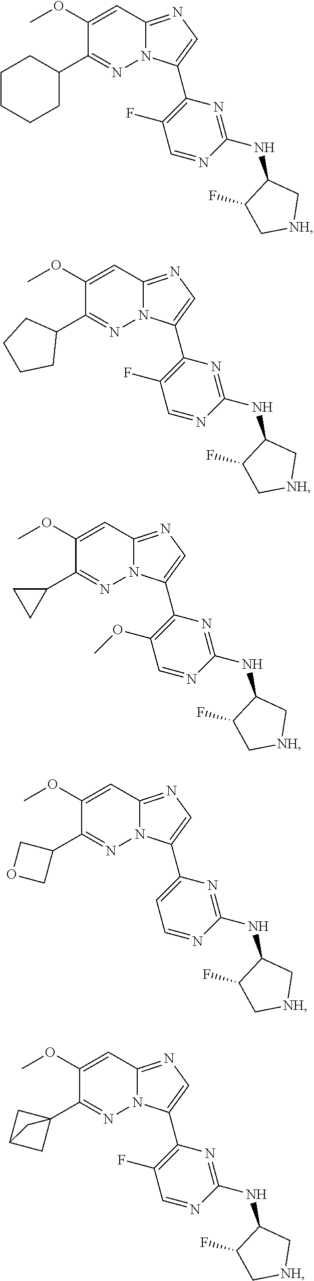 [SMILES: COc1cc2ncc(-c3nc(N[C@H]4CNC[C@@H]4F)ncc3F)n2nc1C1CCCCC1 ; COc1cc2ncc(-c3nc(N[C@H]4CNC[C@@H]4F)ncc3F)n2nc1C1CCCC1 ; COc1cnc(N[C@H]2CNC[C@@H]2F)nc1-c1cnc2cc(OC)c(C3CC3)nn12 ; COc1cc2ncc(-c3ccnc(N[C@H]4CNC[C@@H]4F)n3)n2nc1C1COC1 ; COc1cc2ncc(-c3nc(N[C@H]4CNC[C@@H]4F)ncc3F)n2nc1C12CC(C1)C2]

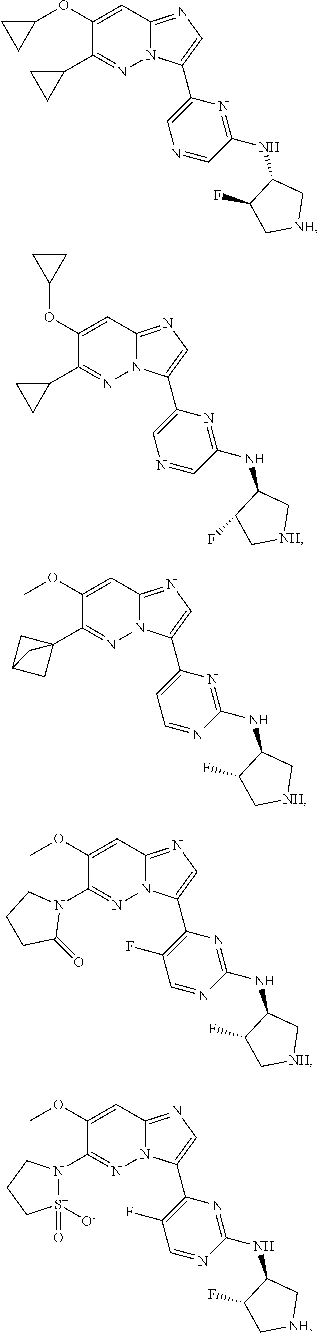 [SMILES: F[C@@H]1CNC[C@H]1Nc1cncc(-c2cnc3cc(OC4CC4)c(C4CC4)nn23)n1 ; F[C@H]1CNC[C@@H]1Nc1cncc(-c2cnc3cc(OC4CC4)c(C4CC4)nn23)n1 ; COc1cc2ncc(-c3ccnc(N[C@H]4CNC[C@@H]4F)n3)n2nc1C12CC(C1)C2 ; COc1cc2ncc(-c3nc(N[C@H]4CNC[C@@H]4F)ncc3F)n2nc1N1CCCC1=O ; COc1cc2ncc(-c3nc(N[C@H]4CNC[C@@H]4F)ncc3F)n2nc1N1CCC[S+]1(=O)[O-]]

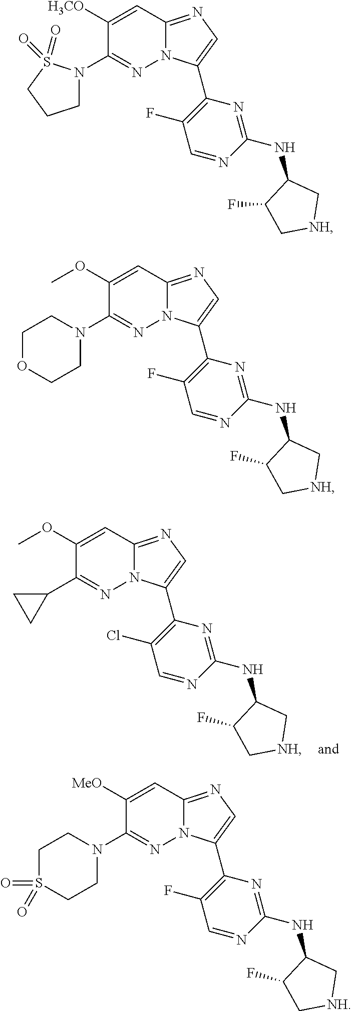 [SMILES: COc1cc2ncc(-c3nc(N[C@H]4CNC[C@@H]4F)ncc3F)n2nc1N1CCCS1(=O)=O ; COc1cc2ncc(-c3nc(N[C@H]4CNC[C@@H]4F)ncc3F)n2nc1N1CCOCC1 ; COc1cc2ncc(-c3nc(N[C@H]4CNC[C@@H]4F)ncc3Cl)n2nc1C1CC1 ; COc1cc2ncc(-c3nc(N[C@H]4CNC[C@@H]4F)ncc3F)n2nc1N1CCS(=O)(=O)CC1]

In an embodiment, the compound of Formula (IIa-5010-WO50) is selected from:

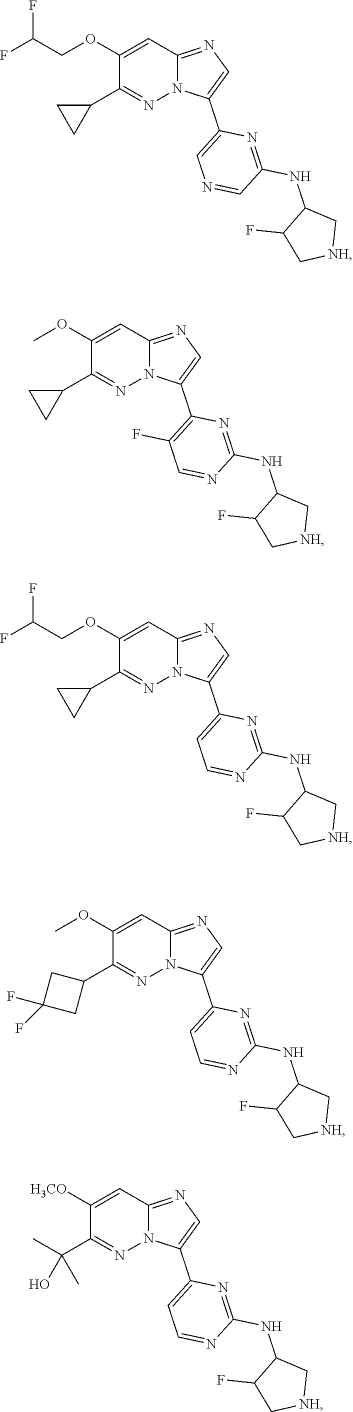 [SMILES: FC(F)COc1cc2ncc(-c3cncc(NC4CNCC4F)n3)n2nc1C1CC1 ; COc1cc2ncc(-c3nc(NC4CNCC4F)ncc3F)n2nc1C1CC1 ; FC(F)COc1cc2ncc(-c3ccnc(NC4CNCC4F)n3)n2nc1C1CC1 ; COc1cc2ncc(-c3ccnc(NC4CNCC4F)n3)n2nc1C1CC(F)(F)C1 ; COc1cc2ncc(-c3ccnc(NC4CNCC4F)n3)n2nc1C(C)(C)O]

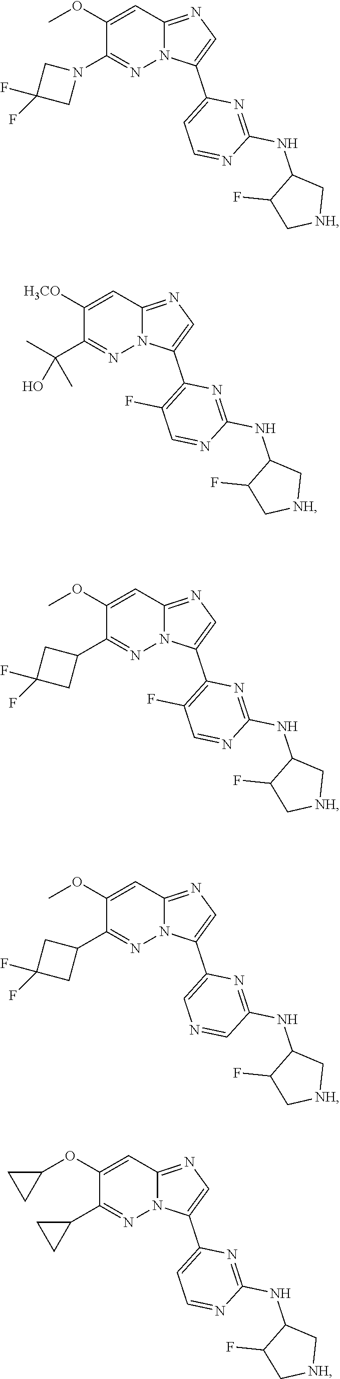 [SMILES: COc1cc2ncc(-c3ccnc(NC4CNCC4F)n3)n2nc1N1CC(F)(F)C1 ; COc1cc2ncc(-c3nc(NC4CNCC4F)ncc3F)n2nc1C(C)(C)O ; COc1cc2ncc(-c3nc(NC4CNCC4F)ncc3F)n2nc1C1CC(F)(F)C1 ; COc1cc2ncc(-c3cncc(NC4CNCC4F)n3)n2nc1C1CC(F)(F)C1 ; FC1CNCC1Nc1nccc(-c2cnc3cc(OC4CC4)c(C4CC4)nn23)n1]

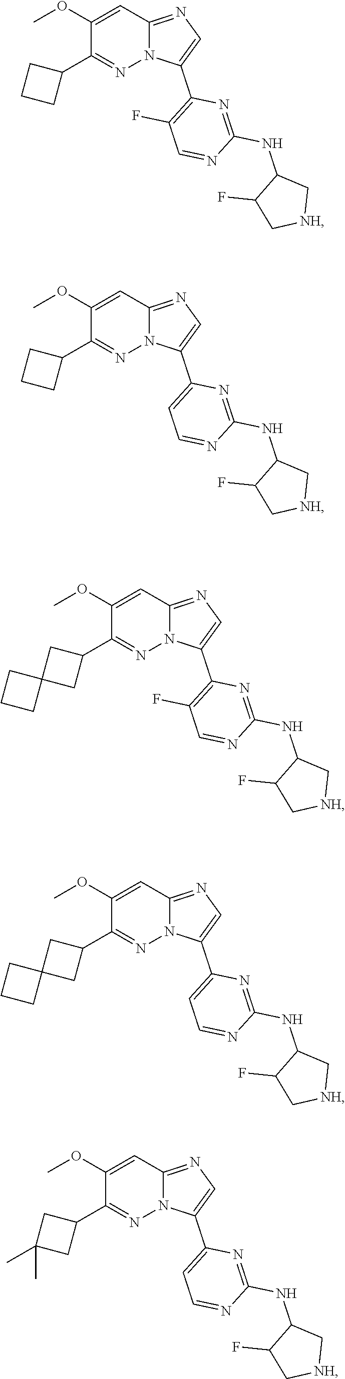 [SMILES: COc1cc2ncc(-c3nc(NC4CNCC4F)ncc3F)n2nc1C1CCC1 ; COc1cc2ncc(-c3ccnc(NC4CNCC4F)n3)n2nc1C1CCC1 ; COc1cc2ncc(-c3nc(NC4CNCC4F)ncc3F)n2nc1C1CC2(CCC2)C1 ; COc1cc2ncc(-c3ccnc(NC4CNCC4F)n3)n2nc1C1CC2(CCC2)C1 ; COc1cc2ncc(-c3ccnc(NC4CNCC4F)n3)n2nc1C1CC(C)(C)C1]

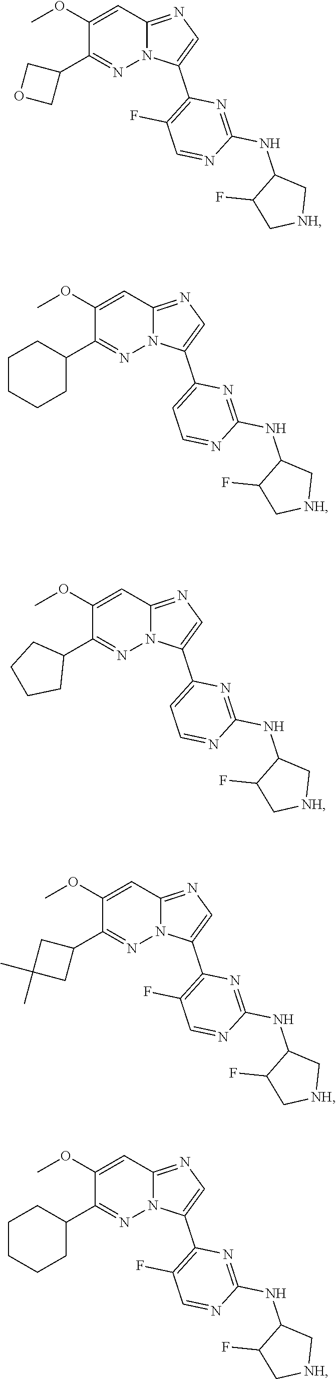 [SMILES: COc1cc2ncc(-c3nc(NC4CNCC4F)ncc3F)n2nc1C1COC1 ; COc1cc2ncc(-c3ccnc(NC4CNCC4F)n3)n2nc1C1CCCCC1 ; COc1cc2ncc(-c3ccnc(NC4CNCC4F)n3)n2nc1C1CCCC1 ; COc1cc2ncc(-c3nc(NC4CNCC4F)ncc3F)n2nc1C1CC(C)(C)C1 ; COc1cc2ncc(-c3nc(NC4CNCC4F)ncc3F)n2nc1C1CCCCC1]

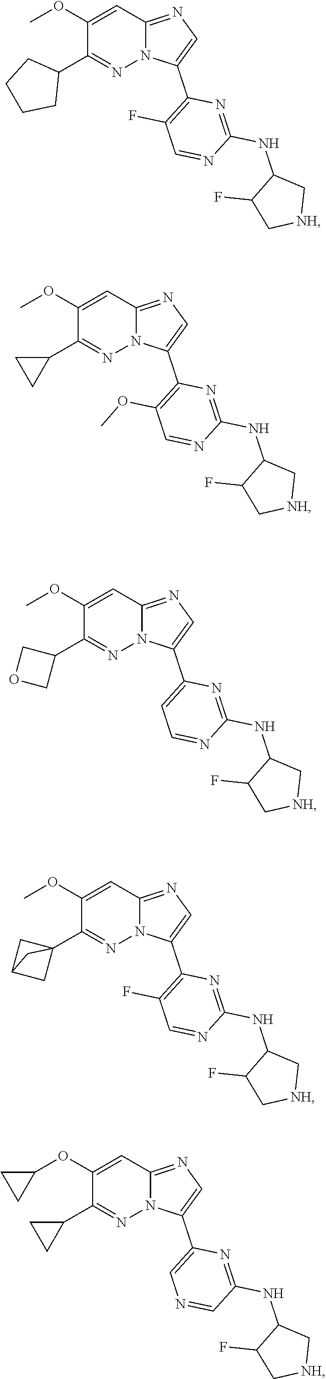 [SMILES: COc1cc2ncc(-c3nc(NC4CNCC4F)ncc3F)n2nc1C1CCCC1 ; COc1cnc(NC2CNCC2F)nc1-c1cnc2cc(OC)c(C3CC3)nn12 ; COc1cc2ncc(-c3ccnc(NC4CNCC4F)n3)n2nc1C1COC1 ; COc1cc2ncc(-c3nc(NC4CNCC4F)ncc3F)n2nc1C12CC(C1)C2 ; FC1CNCC1Nc1cncc(-c2cnc3cc(OC4CC4)c(C4CC4)nn23)n1]

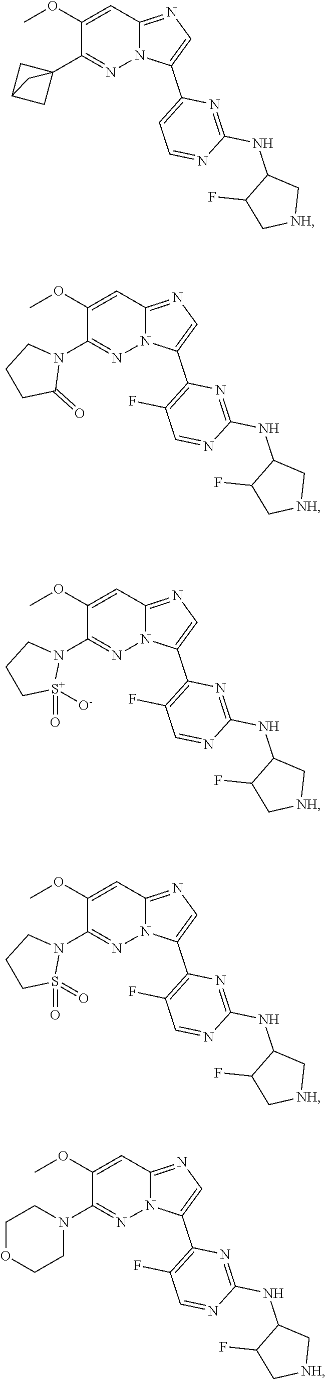 [SMILES: COc1cc2ncc(-c3ccnc(NC4CNCC4F)n3)n2nc1C12CC(C1)C2 ; COc1cc2ncc(-c3nc(NC4CNCC4F)ncc3F)n2nc1N1CCCC1=O ; COc1cc2ncc(-c3nc(NC4CNCC4F)ncc3F)n2nc1N1CCC[S+]1(=O)[O-] ; COc1cc2ncc(-c3nc(NC4CNCC4F)ncc3F)n2nc1N1CCCS1(=O)=O ; COc1cc2ncc(-c3nc(NC4CNCC4F)ncc3F)n2nc1N1CCOCC1]

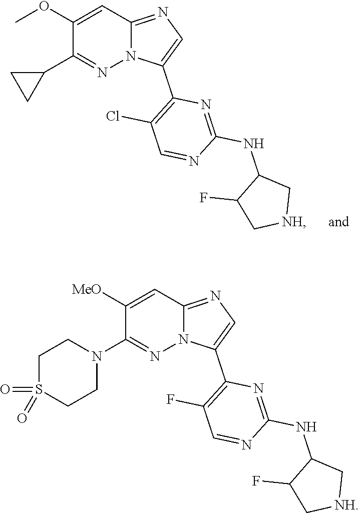 [SMILES: COc1cc2ncc(-c3nc(NC4CNCC4F)ncc3Cl)n2nc1C1CC1 ; COc1cc2ncc(-c3nc(NC4CNCC4F)ncc3F)n2nc1N1CCS(=O)(=O)CC1]

### Formula (IIb-5010-WO)

In another embodiment, the compound of Formula (II) is a compound of Formula (IIb-5010-WO):

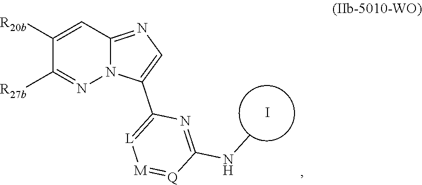

or a salt, ester, solvate, optical isomer, geometric isomer, or salt of an isomer thereof;
   wherein:
   L is N or CR₂₁;
   M is N or CR₂₂;
   Q is N or CR₂₃;

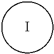

is

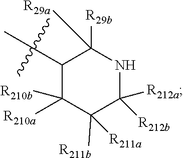

R_{20b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R_{27b} is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₂₁, R₂₂, and R₂₃ are each independently selected from H and halogen;
R₂₈ₐ, R_{28b}, R₂₉ₐ, R_{29b}, R₂₁₀ₐ, R_{210b}, R₂₁₁ₐ, R_{211b}, R₂₁₂ₐ, and R_{212b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of L, M, or Q is N.

In an embodiment, each of R₂₉ₐ, R_{29b}, R₂₁₀ₐ, R_{210b}, R₂₁₁ₐ, R_{211b}, R₂₁₂ₐ, and R_{212b} in Formula (IIb-5010-WO) is H. In another embodiment, one or more of R₂₉ₐ, R_{29b}, R₂₁₀ₐ, R_{210b}, R₂₁₁ₐ, R_{211b}, R₂₁₂ₐ, and R_{212b} is selected from halogen, —OH, optionally substituted C₁-C₆ alkyl, and optionally substituted C₁-C₆ alkoxy. In one embodiment, each of R₂₉ₐ, R_{29b}, R₂₁₁ₐ, R_{211b}, R₂₁₂ₐ, and R_{212b} is H and R₂₁₀ₐ and/or R_{210b} is halogen. In one embodiment, each of R₂₉ₐ, R_{29b}, R₂₁₁ₐ, R_{211b}, R₂₁₂ₐ, and R_{212b} is H and R₂₁₀ₐ and/or R_{210b} is F. In one embodiment, each of R₂₉ₐ, R_{29b}, R_{210b}, R₂₁₁ₐ, R_{211b}, R₂₁₂ₐ, and R_{212b} is H and R₂₁₀ₐ is F.

In an embodiment, R_{20b} in Formula (IIb-5010-WO) is not H. In an embodiment, R_{20b} is unsubstituted C₁-C₆ alkoxy. In one embodiment, R_{20b} is —OCH₃.

In an embodiment, R_{27b} of Formula (IIb-5010-WO) is unsubstituted C₃-C₆ cycloalkyl. In one embodiment, R_{27b} is unsubstituted C₃ cycloalkyl. In an embodiment, R_{27b} is C₁-C₆ alkyl substituted with one —OH and/or halogen. In one embodiment, R_{27b} is

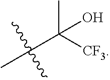 [SMILES: *C(C)(O)C(F)(F)F]

In an embodiment, the compound of Formula (IIb-5010-WO) is a compound covered by the above formula and shown in Table 1 of the present application.

### Formula (IIb-5010-WO50)

In another embodiment, the compound of Formula (II) is a compound of Formula (IIb-5010-WO50):

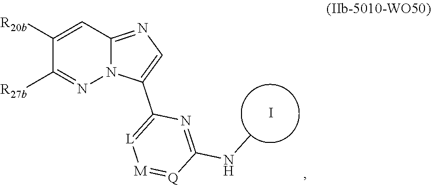

or a salt, ester, solvate, optical isomer, geometric isomer, or salt of an isomer thereof;
   wherein:
   L is N or CR₂₁;
   M is N or CR₂₂;
   Q is N or CR₂₃;

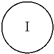

is

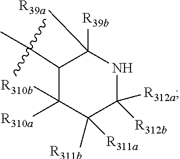

R_{20b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from C₁-C₆ alkyl and halogen;
R_{27b} is selected from halogen, C₁-C₆ alkyl, C₁-C₆ alkoxy, C₃-C₆ cycloalkyl, spiro-cycloalkyl, —O—(C₃-C₆ cycloalkyl), C₂-C₆ heterocyclyl, imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl, spiro-cycloalkyl, —O—(C₃-C₆ cycloalkyl), and C₂-C₆ heterocyclyl are each optionally substituted with one or more substituents selected from —OH, C₁-C₆ alkyl, and halogen,
R₂₁, R₂₂, and R₂₃ are each independently selected from H, C₁-C₆ alkoxy, and halogen;
R₂₉ₐ, R_{29b}, R₂₁₀ₐ, R_{210b}, R₂₁₁ₐ, R_{211b}, R₂₁₂ₐ, and R_{212b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms;
R₂₈ₐ and R_{28b} are each independently selected from H, C₁-C₆ alkyl, and C₃-C₆ cycloalkyl, wherein C₁-C₆ alkyl and C₃-C₆ cycloalkyl are each optionally substituted with one or more halogen; and
one of L, M, or Q is N.

In an embodiment, each of R₂₉ₐ, R_{29b}, R₂₁₀ₐ, R_{210b}, R₂₁₁ₐ, R_{211b}, R₂₁₂ₐ, and R_{212b} in Formula (IIb-5010-WO50) is H. In another embodiment, one or more of R₂₉ₐ, R_{29b}, R₂₁₀ₐ, R_{210b}, R₂₁₁ₐ, R_{211b}, R₂₁₂ₐ, and R_{212b} is selected from halogen, —OH, optionally substituted C₁-C₆ alkyl, and optionally substituted C₁-C₆ alkoxy. In one embodiment, each of R₂₉ₐ, R_{29b}, R₂₁₁ₐ, R_{211b}, R₂₁₂ₐ, and R_{212b} is H and R₂₁₀ₐ and/or R_{210b} is halogen. In one embodiment, each of R₂₉ₐ, R_{29b}, R₂₁₁ₐ, R_{211b}, R₂₁₂ₐ, and R_{212b} is H and R₂₁₀ₐ and/or R_{210b} is F. In one embodiment, each of R₂₉ₐ, R_{29b}, R_{210b}, R₂₁₁ₐ, R_{211b}, R₂₁₂ₐ, and R_{212b} is H and R₂₁₀ₐ is F.

In an embodiment, R_{20b} in Formula (IIb-5010-WO50) is not H. In an embodiment, R_{20b} is unsubstituted C₁-C₆ alkoxy. In one embodiment, R_{20b} is —OCH₃. In one embodiment, R_{20b} is —OCD₃. In another embodiment, R_{20b} is C₁-C₆ alkoxy substituted with one or more F. In one embodiment, R_{20b} is

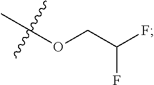 [SMILES: *OCC(F)F]

In another embodiment, R_{20b} is unsubstituted —O—(C₃-C₆ cycloalkyl). In one embodiment, R_{20b} is unsubstituted —O—(C₃ cycloalkyl).

In one embodiment, R₂₁, R₂₂, and R₂₃ of Formula (IIb-5010-WO50), if present, are H. In another embodiment, L is CR₂₁ wherein R₂₁ is selected from F, Cl, and —OCH₃, M is CR₂₂ wherein R₂₂ is H, and Q is N;

In an embodiment, R_{27b} of Formula (IIb-5010-WO50) is unsubstituted C₃-C₆ cycloalkyl. In one embodiment, R_{27b} is unsubstituted C₃ cycloalkyl or

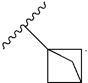 [SMILES: *C12CC(C1)C2]

In an embodiment, R_{27b} is C₁-C₆ alkyl substituted with one or more —OH and/or one or halogen. In one embodiment, R_{27b} is

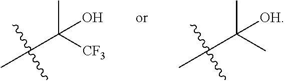

In another embodiment, R_{27b} is C₃-C₆ cycloalkyl substituted with one or more —CH₃, —OH, and/or F. In one embodiment, R_{27b} is selected from

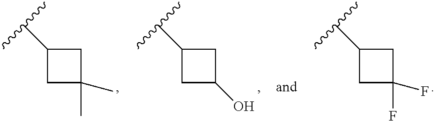

In one embodiment, R_{27b} is spiro-fused cycloalkyl. In one embodiment, R_{27b} is

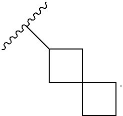 [SMILES: *C1CC2(CCC2)C1]

In another embodiment, R_{27b} is unsubstituted C₂-C₆ heterocyclyl. In one embodiment, R_{27b} is unsubstituted C₂-C₆ heterocyclyl comprising one oxygen atom, one nitrogen atom, or one oxygen atom and one nitrogen atom. In one embodiment, R_{27b} is selected from

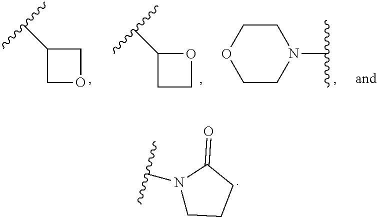

In one embodiment, R_{27b} is

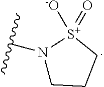

In one embodiment, R_{27b} is

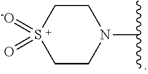

In another embodiment, R_{27b} is a C₂-C₆ heterocyclyl substituted with one or more F and/or —CH₃. In one embodiment, R_{27b} is

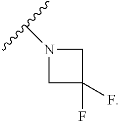 [SMILES: *N1CC(F)(F)C1]

In one embodiment, R_{27b} is o

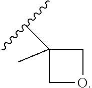 [SMILES: *C1(C)COC1]

In one embodiment, the compound of Formula (II) is a compound of Formula (IIb-5010-WO50) with the proviso that when R_{20b} is —OCH₃ and R_{27b} is unsubstituted C₃ cycloalkyl, at least one of R₂₁, R₂₂, and R₂₃ is present and selected from C₁-C₆ alkoxy and halogen.

In one embodiment, the compound of Formula (IIb-5010-WO50) is not:

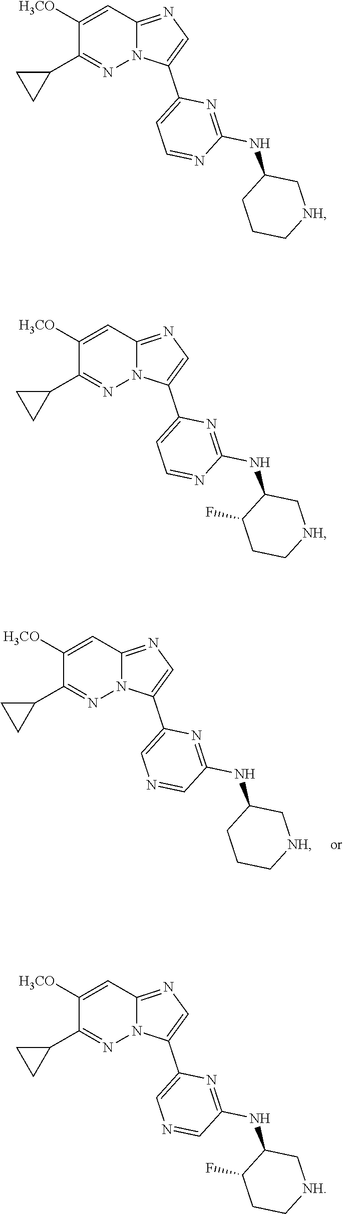 [SMILES: COc1cc2ncc(-c3ccnc(N[C@@H]4CCCNC4)n3)n2nc1C1CC1 ; COc1cc2ncc(-c3ccnc(N[C@H]4CNCC[C@@H]4F)n3)n2nc1C1CC1 ; COc1cc2ncc(-c3cncc(N[C@@H]4CCCNC4)n3)n2nc1C1CC1 ; COc1cc2ncc(-c3cncc(N[C@H]4CNCC[C@@H]4F)n3)n2nc1C1CC1]

In an embodiment, the compound of Formula (IIb-5010-WO50) is selected from:

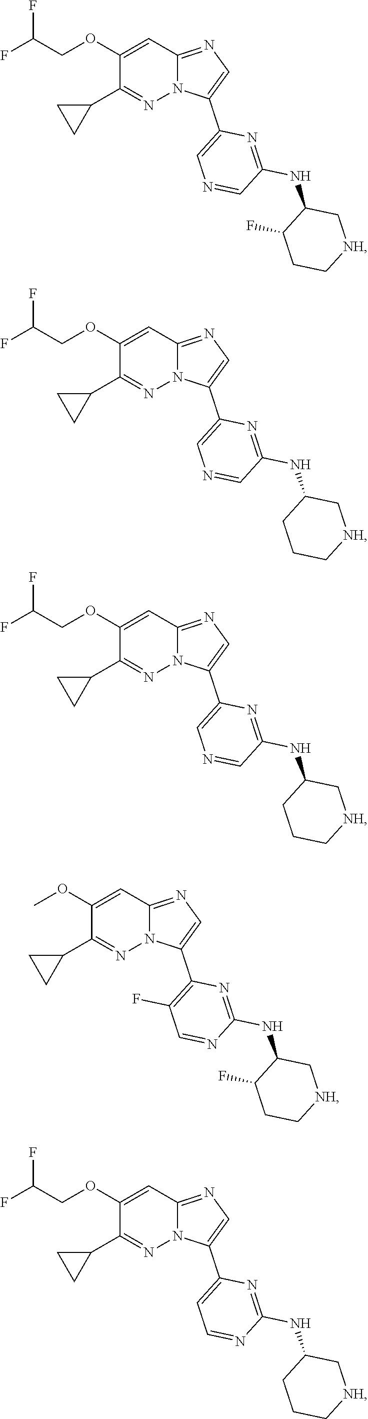 [SMILES: FC(F)COc1cc2ncc(-c3cncc(N[C@H]4CNCC[C@@H]4F)n3)n2nc1C1CC1 ; FC(F)COc1cc2ncc(-c3cncc(N[C@H]4CCCNC4)n3)n2nc1C1CC1 ; FC(F)COc1cc2ncc(-c3cncc(N[C@@H]4CCCNC4)n3)n2nc1C1CC1 ; COc1cc2ncc(-c3nc(N[C@H]4CNCC[C@@H]4F)ncc3F)n2nc1C1CC1 ; FC(F)COc1cc2ncc(-c3ccnc(N[C@H]4CCCNC4)n3)n2nc1C1CC1]

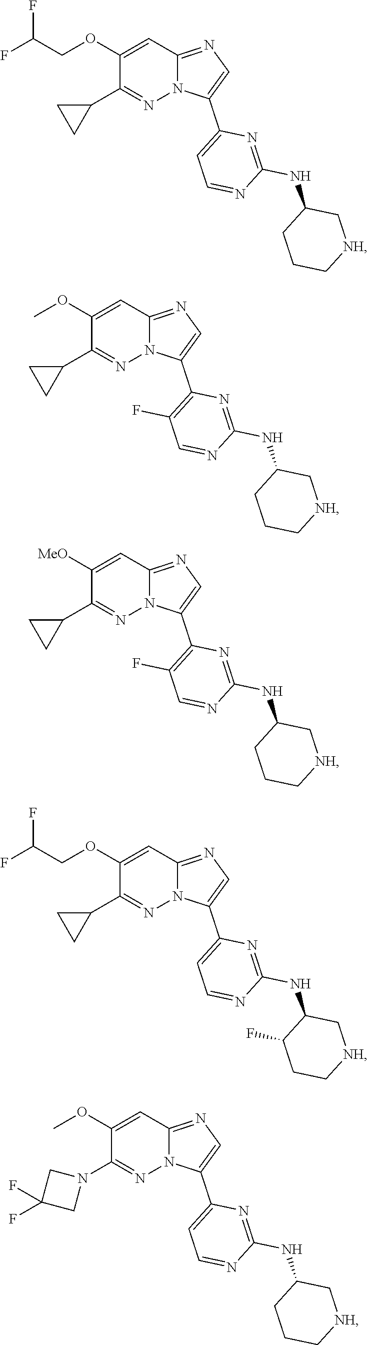 [SMILES: FC(F)COc1cc2ncc(-c3ccnc(N[C@@H]4CCCNC4)n3)n2nc1C1CC1 ; COc1cc2ncc(-c3nc(N[C@H]4CCCNC4)ncc3F)n2nc1C1CC1 ; COc1cc2ncc(-c3nc(N[C@@H]4CCCNC4)ncc3F)n2nc1C1CC1 ; FC(F)COc1cc2ncc(-c3ccnc(N[C@H]4CNCC[C@@H]4F)n3)n2nc1C1CC1 ; COc1cc2ncc(-c3ccnc(N[C@H]4CCCNC4)n3)n2nc1N1CC(F)(F)C1]

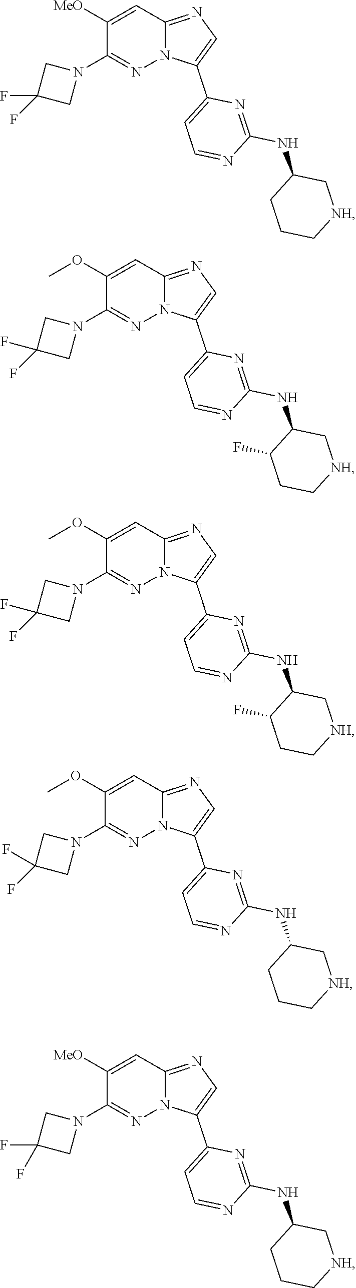 [SMILES: COc1cc2ncc(-c3ccnc(N[C@@H]4CCCNC4)n3)n2nc1N1CC(F)(F)C1 ; COc1cc2ncc(-c3ccnc(N[C@H]4CNCC[C@@H]4F)n3)n2nc1N1CC(F)(F)C1 ; COc1cc2ncc(-c3ccnc(N[C@H]4CNCC[C@@H]4F)n3)n2nc1N1CC(F)(F)C1 ; COc1cc2ncc(-c3ccnc(N[C@H]4CCCNC4)n3)n2nc1N1CC(F)(F)C1 ; COc1cc2ncc(-c3ccnc(N[C@@H]4CCCNC4)n3)n2nc1N1CC(F)(F)C1]

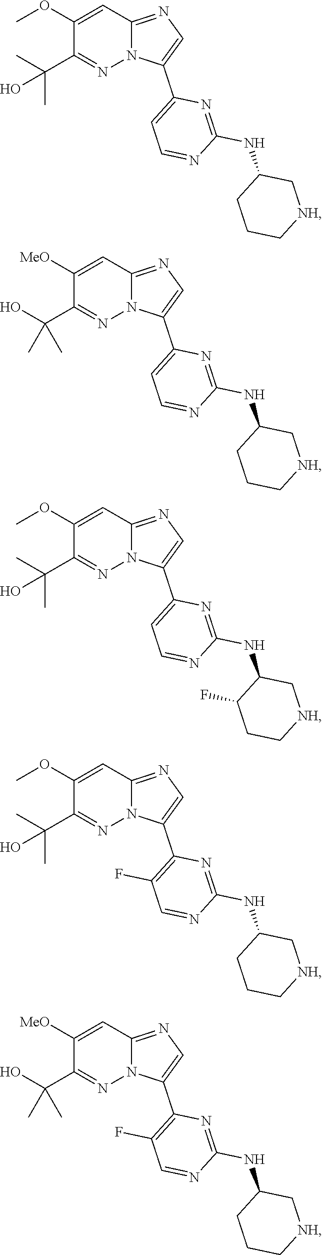 [SMILES: COc1cc2ncc(-c3ccnc(N[C@H]4CCCNC4)n3)n2nc1C(C)(C)O ; COc1cc2ncc(-c3ccnc(N[C@@H]4CCCNC4)n3)n2nc1C(C)(C)O ; COc1cc2ncc(-c3ccnc(N[C@H]4CNCC[C@@H]4F)n3)n2nc1C(C)(C)O ; COc1cc2ncc(-c3nc(N[C@H]4CCCNC4)ncc3F)n2nc1C(C)(C)O ; COc1cc2ncc(-c3nc(N[C@@H]4CCCNC4)ncc3F)n2nc1C(C)(C)O]

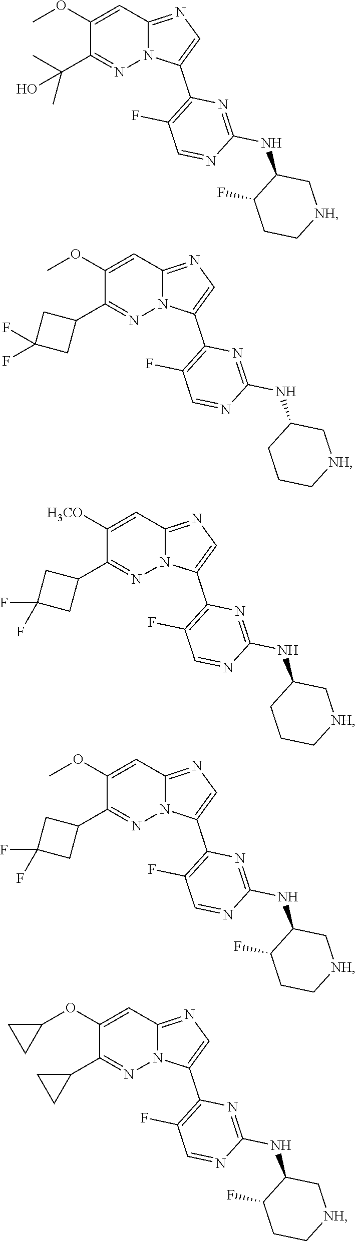 [SMILES: COc1cc2ncc(-c3nc(N[C@H]4CNCC[C@@H]4F)ncc3F)n2nc1C(C)(C)O ; COc1cc2ncc(-c3nc(N[C@H]4CCCNC4)ncc3F)n2nc1C1CC(F)(F)C1 ; COc1cc2ncc(-c3nc(N[C@@H]4CCCNC4)ncc3F)n2nc1C1CC(F)(F)C1 ; COc1cc2ncc(-c3nc(N[C@H]4CNCC[C@@H]4F)ncc3F)n2nc1C1CC(F)(F)C1 ; Fc1cnc(N[C@H]2CNCC[C@@H]2F)nc1-c1cnc2cc(OC3CC3)c(C3CC3)nn12]

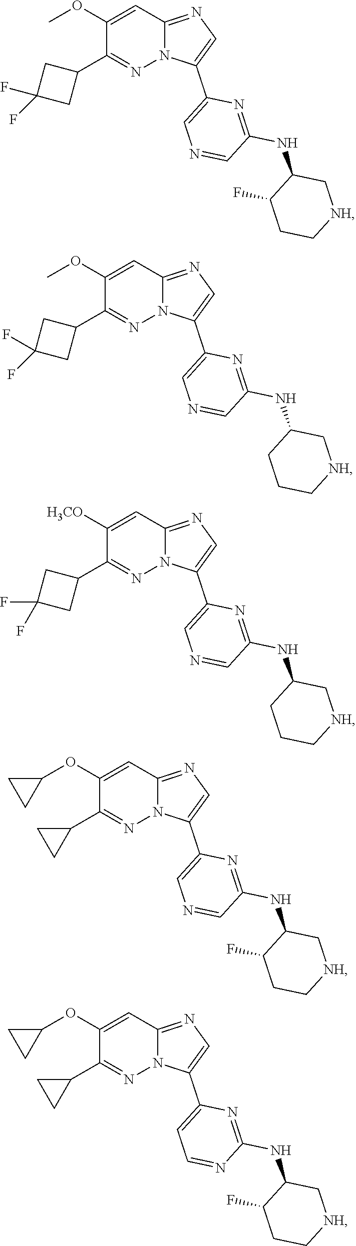 [SMILES: COc1cc2ncc(-c3cncc(N[C@H]4CNCC[C@@H]4F)n3)n2nc1C1CC(F)(F)C1 ; COc1cc2ncc(-c3cncc(N[C@H]4CCCNC4)n3)n2nc1C1CC(F)(F)C1 ; COc1cc2ncc(-c3cncc(N[C@@H]4CCCNC4)n3)n2nc1C1CC(F)(F)C1 ; F[C@H]1CCNC[C@@H]1Nc1cncc(-c2cnc3cc(OC4CC4)c(C4CC4)nn23)n1 ; F[C@H]1CCNC[C@@H]1Nc1nccc(-c2cnc3cc(OC4CC4)c(C4CC4)nn23)n1]

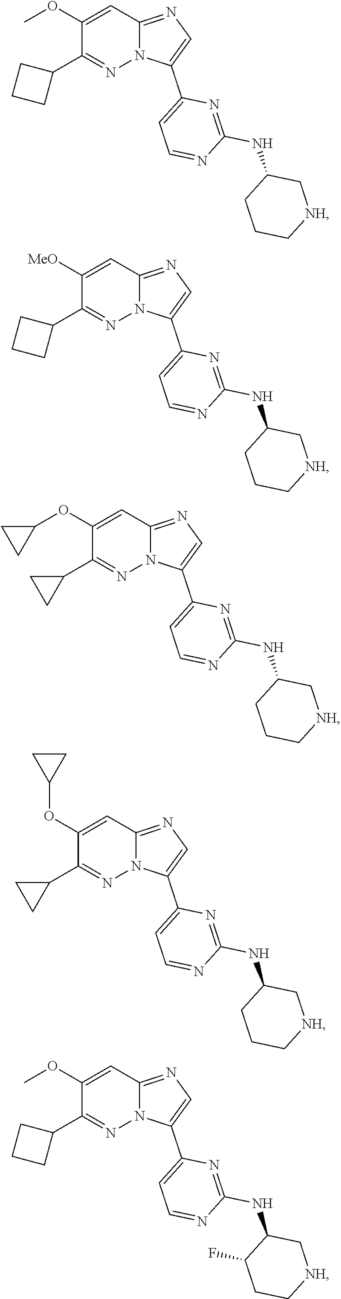 [SMILES: COc1cc2ncc(-c3ccnc(N[C@H]4CCCNC4)n3)n2nc1C1CCC1 ; COc1cc2ncc(-c3ccnc(N[C@@H]4CCCNC4)n3)n2nc1C1CCC1 ; c1cc(-c2cnc3cc(OC4CC4)c(C4CC4)nn23)nc(N[C@H]2CCCNC2)n1 ; c1cc(-c2cnc3cc(OC4CC4)c(C4CC4)nn23)nc(N[C@@H]2CCCNC2)n1 ; COc1cc2ncc(-c3ccnc(N[C@H]4CNCC[C@@H]4F)n3)n2nc1C1CCC1]

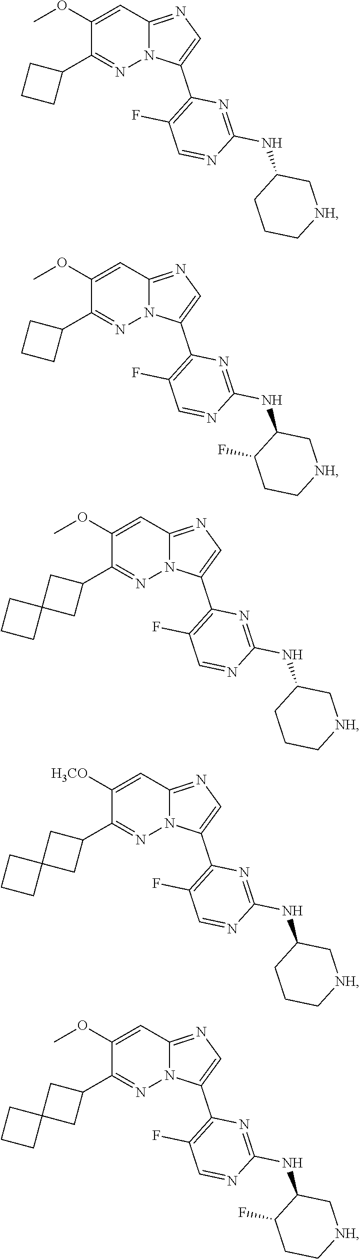 [SMILES: COc1cc2ncc(-c3nc(N[C@H]4CCCNC4)ncc3F)n2nc1C1CCC1 ; COc1cc2ncc(-c3nc(N[C@H]4CNCC[C@@H]4F)ncc3F)n2nc1C1CCC1 ; COc1cc2ncc(-c3nc(N[C@H]4CCCNC4)ncc3F)n2nc1C1CC2(CCC2)C1 ; COc1cc2ncc(-c3nc(N[C@@H]4CCCNC4)ncc3F)n2nc1C1CC2(CCC2)C1 ; COc1cc2ncc(-c3nc(N[C@H]4CNCC[C@@H]4F)ncc3F)n2nc1C1CC2(CCC2)C1]

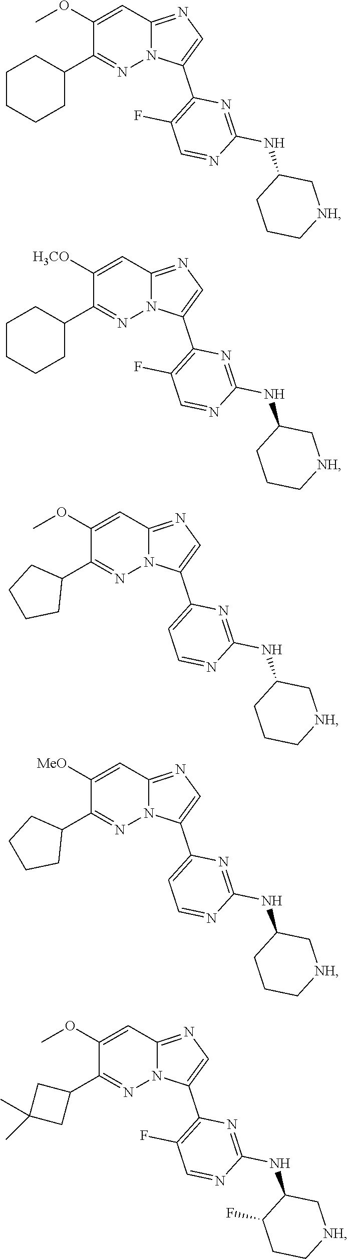 [SMILES: COc1cc2ncc(-c3nc(N[C@H]4CCCNC4)ncc3F)n2nc1C1CCCCC1 ; COc1cc2ncc(-c3nc(N[C@@H]4CCCNC4)ncc3F)n2nc1C1CCCCC1 ; COc1cc2ncc(-c3ccnc(N[C@H]4CCCNC4)n3)n2nc1C1CCCC1 ; COc1cc2ncc(-c3ccnc(N[C@@H]4CCCNC4)n3)n2nc1C1CCCC1 ; COc1cc2ncc(-c3nc(N[C@H]4CNCC[C@@H]4F)ncc3F)n2nc1C1CC(C)(C)C1]

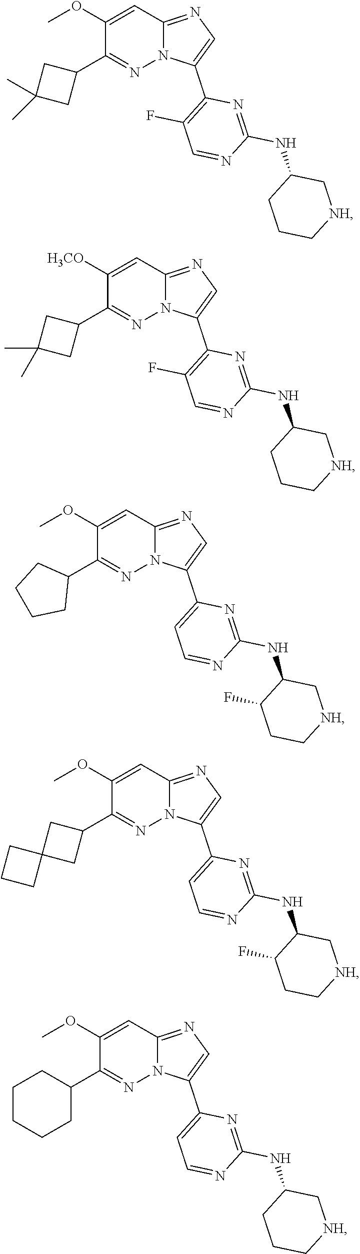 [SMILES: COc1cc2ncc(-c3nc(N[C@H]4CCCNC4)ncc3F)n2nc1C1CC(C)(C)C1 ; COc1cc2ncc(-c3nc(N[C@@H]4CCCNC4)ncc3F)n2nc1C1CC(C)(C)C1 ; COc1cc2ncc(-c3ccnc(N[C@H]4CNCC[C@@H]4F)n3)n2nc1C1CCCC1 ; COc1cc2ncc(-c3ccnc(N[C@H]4CNCC[C@@H]4F)n3)n2nc1C1CC2(CCC2)C1 ; COc1cc2ncc(-c3ccnc(N[C@H]4CCCNC4)n3)n2nc1C1CCCCC1]

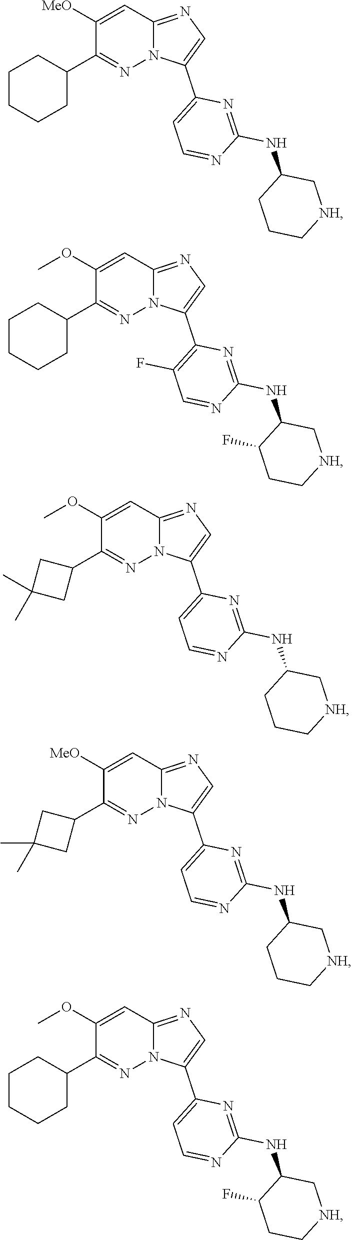 [SMILES: COc1cc2ncc(-c3ccnc(N[C@@H]4CCCNC4)n3)n2nc1C1CCCCC1 ; COc1cc2ncc(-c3nc(N[C@H]4CNCC[C@@H]4F)ncc3F)n2nc1C1CCCCC1 ; COc1cc2ncc(-c3ccnc(N[C@H]4CCCNC4)n3)n2nc1C1CC(C)(C)C1 ; COc1cc2ncc(-c3ccnc(N[C@@H]4CCCNC4)n3)n2nc1C1CC(C)(C)C1 ; COc1cc2ncc(-c3ccnc(N[C@H]4CNCC[C@@H]4F)n3)n2nc1C1CCCCC1]

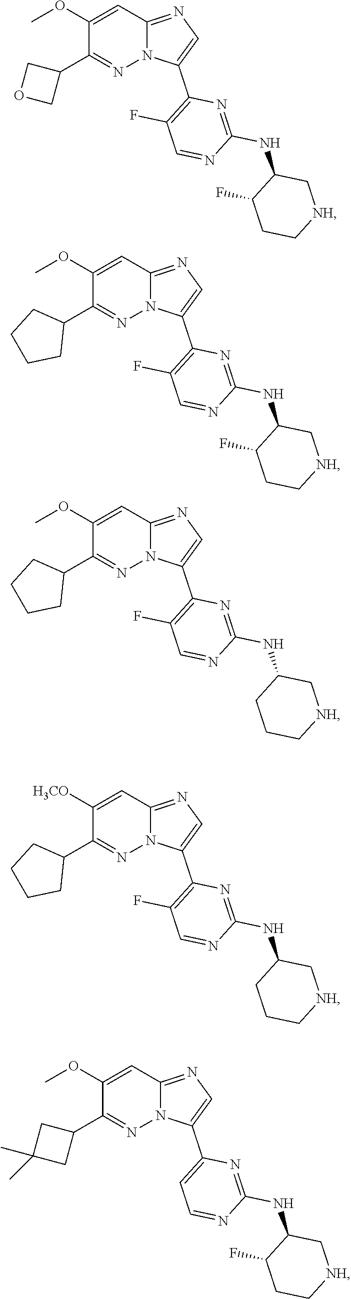 [SMILES: COc1cc2ncc(-c3nc(N[C@H]4CNCC[C@@H]4F)ncc3F)n2nc1C1COC1 ; COc1cc2ncc(-c3nc(N[C@H]4CNCC[C@@H]4F)ncc3F)n2nc1C1CCCC1 ; COc1cc2ncc(-c3nc(N[C@H]4CCCNC4)ncc3F)n2nc1C1CCCC1 ; COc1cc2ncc(-c3nc(N[C@@H]4CCCNC4)ncc3F)n2nc1C1CCCC1 ; COc1cc2ncc(-c3ccnc(N[C@H]4CNCC[C@@H]4F)n3)n2nc1C1CC(C)(C)C1]

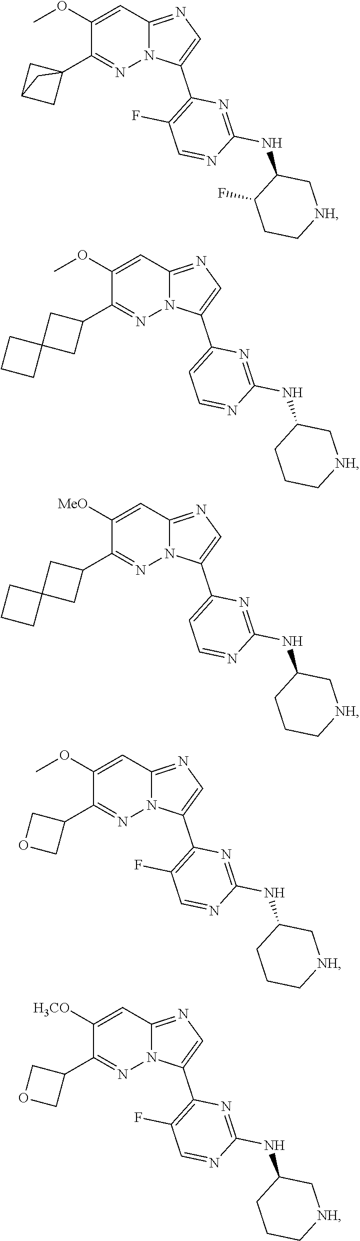 [SMILES: COc1cc2ncc(-c3nc(N[C@H]4CNCC[C@@H]4F)ncc3F)n2nc1C12CC(C1)C2 ; COc1cc2ncc(-c3ccnc(N[C@H]4CCCNC4)n3)n2nc1C1CC2(CCC2)C1 ; COc1cc2ncc(-c3ccnc(N[C@@H]4CCCNC4)n3)n2nc1C1CC2(CCC2)C1 ; COc1cc2ncc(-c3nc(N[C@H]4CCCNC4)ncc3F)n2nc1C1COC1 ; COc1cc2ncc(-c3nc(N[C@@H]4CCCNC4)ncc3F)n2nc1C1COC1]

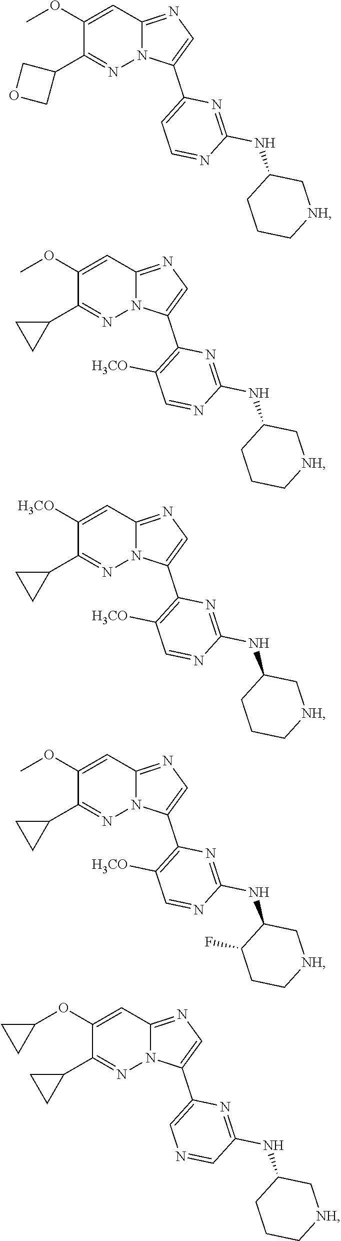 [SMILES: COc1cc2ncc(-c3ccnc(N[C@H]4CCCNC4)n3)n2nc1C1COC1 ; COc1cnc(N[C@H]2CCCNC2)nc1-c1cnc2cc(OC)c(C3CC3)nn12 ; COc1cnc(N[C@@H]2CCCNC2)nc1-c1cnc2cc(OC)c(C3CC3)nn12 ; COc1cnc(N[C@H]2CNCC[C@@H]2F)nc1-c1cnc2cc(OC)c(C3CC3)nn12 ; c1ncc(-c2cnc3cc(OC4CC4)c(C4CC4)nn23)nc1N[C@H]1CCCNC1]

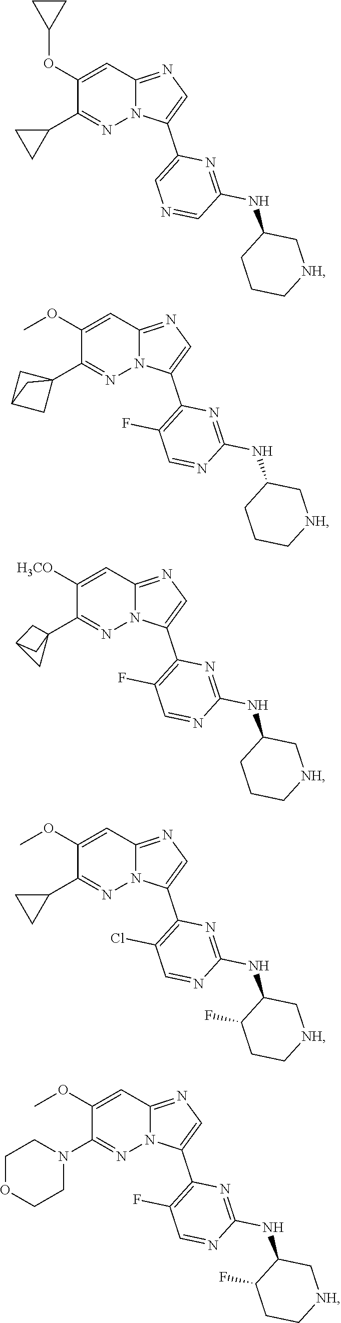 [SMILES: c1ncc(-c2cnc3cc(OC4CC4)c(C4CC4)nn23)nc1N[C@@H]1CCCNC1 ; COc1cc2ncc(-c3nc(N[C@H]4CCCNC4)ncc3F)n2nc1C12CC(C1)C2 ; COc1cc2ncc(-c3nc(N[C@@H]4CCCNC4)ncc3F)n2nc1C12CC(C1)C2 ; COc1cc2ncc(-c3nc(N[C@H]4CNCC[C@@H]4F)ncc3Cl)n2nc1C1CC1 ; COc1cc2ncc(-c3nc(N[C@H]4CNCC[C@@H]4F)ncc3F)n2nc1N1CCOCC1]

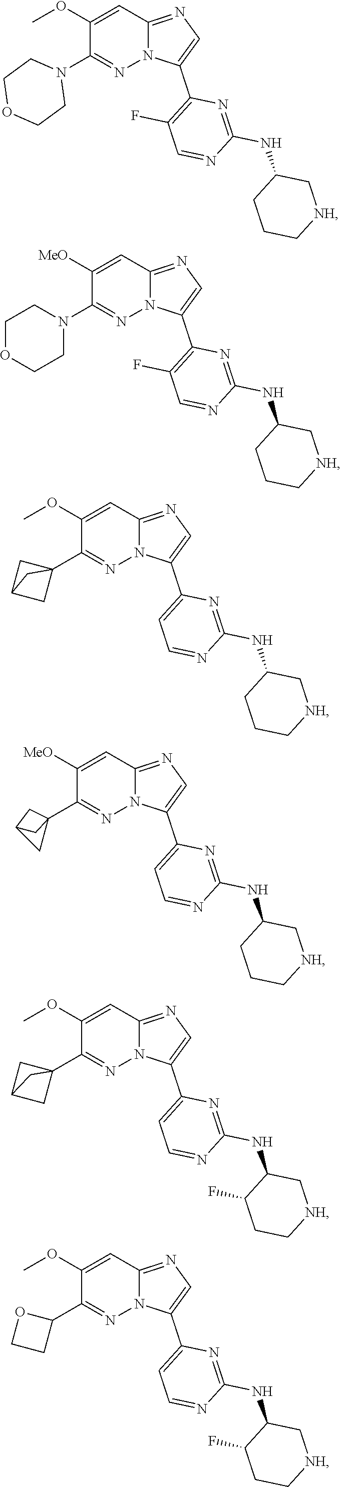 [SMILES: COc1cc2ncc(-c3nc(N[C@H]4CCCNC4)ncc3F)n2nc1N1CCOCC1 ; COc1cc2ncc(-c3nc(N[C@@H]4CCCNC4)ncc3F)n2nc1N1CCOCC1 ; COc1cc2ncc(-c3ccnc(N[C@H]4CCCNC4)n3)n2nc1C12CC(C1)C2 ; COc1cc2ncc(-c3ccnc(N[C@@H]4CCCNC4)n3)n2nc1C12CC(C1)C2 ; COc1cc2ncc(-c3ccnc(N[C@H]4CNCC[C@@H]4F)n3)n2nc1C12CC(C1)C2 ; COc1cc2ncc(-c3ccnc(N[C@H]4CNCC[C@@H]4F)n3)n2nc1C1CCO1]

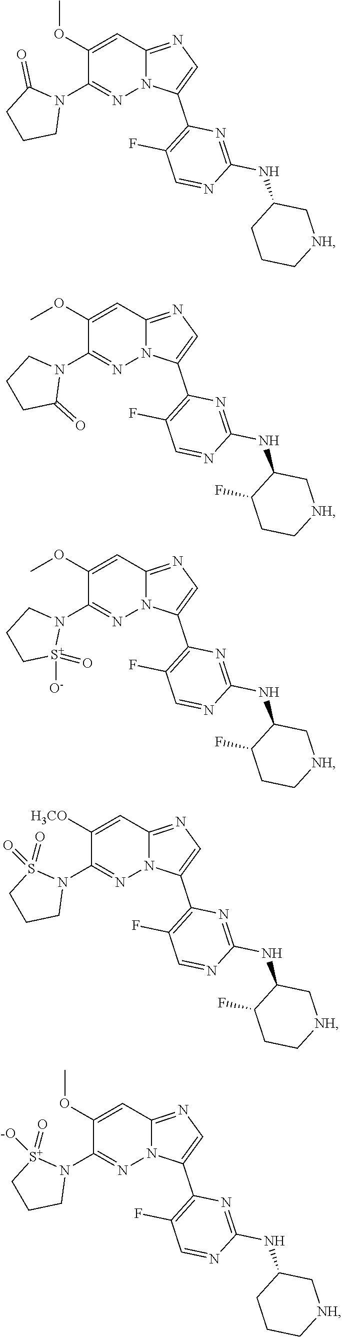

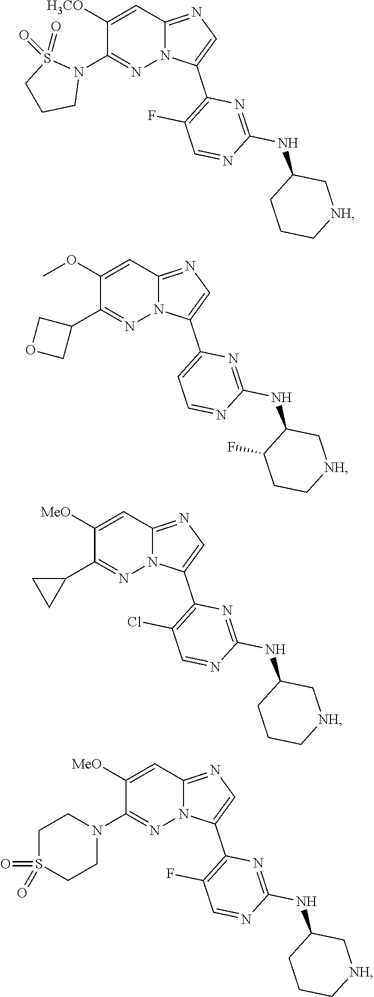 [SMILES: COc1cc2ncc(-c3nc(N[C@@H]4CCCNC4)ncc3F)n2nc1N1CCCS1(=O)=O ; COc1cc2ncc(-c3ccnc(N[C@H]4CNCC[C@@H]4F)n3)n2nc1C1COC1 ; COc1cc2ncc(-c3nc(N[C@@H]4CCCNC4)ncc3Cl)n2nc1C1CC1 ; COc1cc2ncc(-c3nc(N[C@@H]4CCCNC4)ncc3F)n2nc1N1CCS(=O)(=O)CC1]

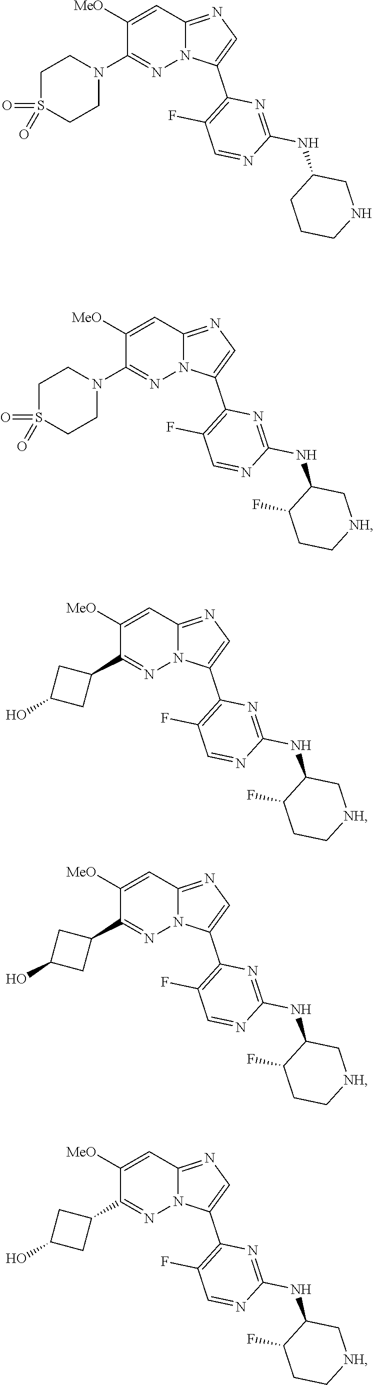 [SMILES: COc1cc2ncc(-c3nc(N[C@H]4CCCNC4)ncc3F)n2nc1N1CCS(=O)(=O)CC1 ; COc1cc2ncc(-c3nc(N[C@H]4CNCC[C@@H]4F)ncc3F)n2nc1N1CCS(=O)(=O)CC1 ; COc1cc2ncc(-c3nc(N[C@H]4CNCC[C@@H]4F)ncc3F)n2nc1[C@H]1C[C@H](O)C1 ; COc1cc2ncc(-c3nc(N[C@H]4CNCC[C@@H]4F)ncc3F)n2nc1[C@H]1C[C@@H](O)C1 ; COc1cc2ncc(-c3nc(N[C@H]4CNCC[C@@H]4F)ncc3F)n2nc1[C@H]1C[C@@H](O)C1]

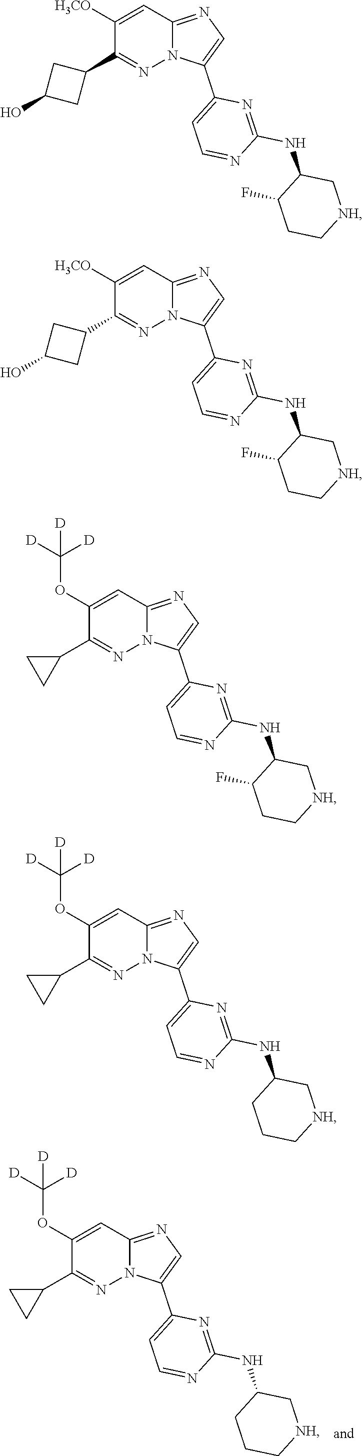

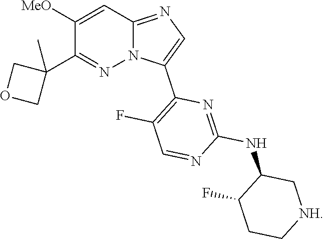 [SMILES: COc1cc2ncc(-c3nc(N[C@H]4CNCC[C@@H]4F)ncc3F)n2nc1C1(C)COC1]

In an embodiment, the compound of Formula (IIb-5010-WO50) is selected from:

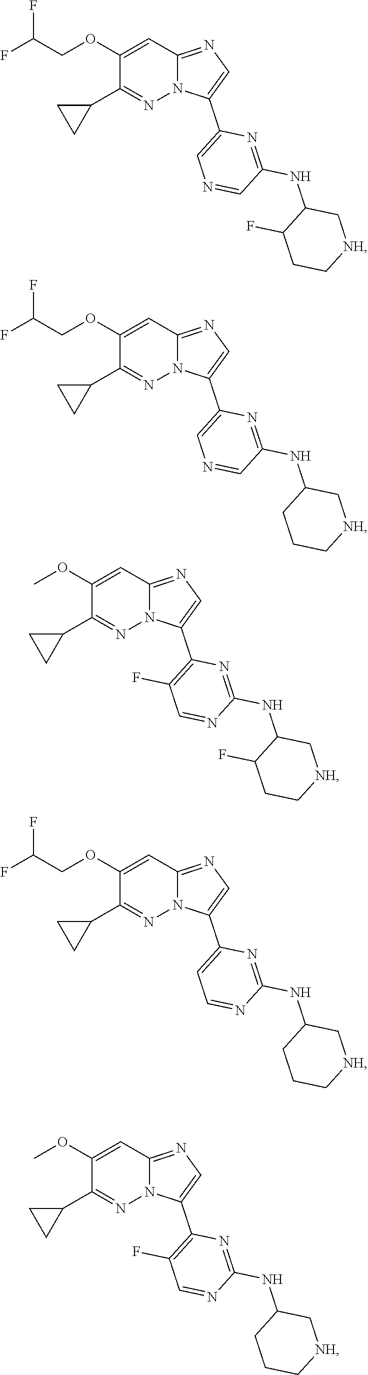 [SMILES: FC(F)COc1cc2ncc(-c3cncc(NC4CNCCC4F)n3)n2nc1C1CC1 ; FC(F)COc1cc2ncc(-c3cncc(NC4CCCNC4)n3)n2nc1C1CC1 ; COc1cc2ncc(-c3nc(NC4CNCCC4F)ncc3F)n2nc1C1CC1 ; FC(F)COc1cc2ncc(-c3ccnc(NC4CCCNC4)n3)n2nc1C1CC1 ; COc1cc2ncc(-c3nc(NC4CCCNC4)ncc3F)n2nc1C1CC1]

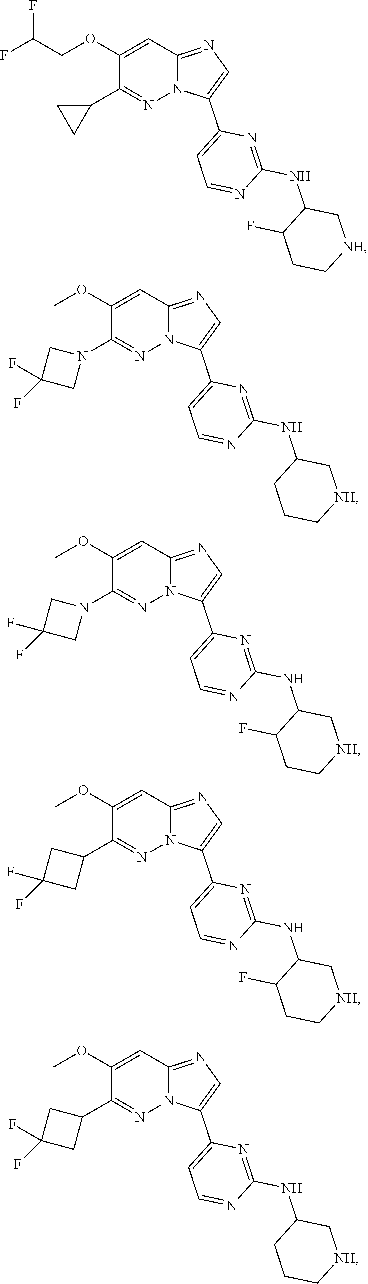 [SMILES: FC(F)COc1cc2ncc(-c3ccnc(NC4CNCCC4F)n3)n2nc1C1CC1 ; COc1cc2ncc(-c3ccnc(NC4CCCNC4)n3)n2nc1N1CC(F)(F)C1 ; COc1cc2ncc(-c3ccnc(NC4CNCCC4F)n3)n2nc1N1CC(F)(F)C1 ; COc1cc2ncc(-c3ccnc(NC4CNCCC4F)n3)n2nc1C1CC(F)(F)C1 ; COc1cc2ncc(-c3ccnc(NC4CCCNC4)n3)n2nc1C1CC(F)(F)C1]

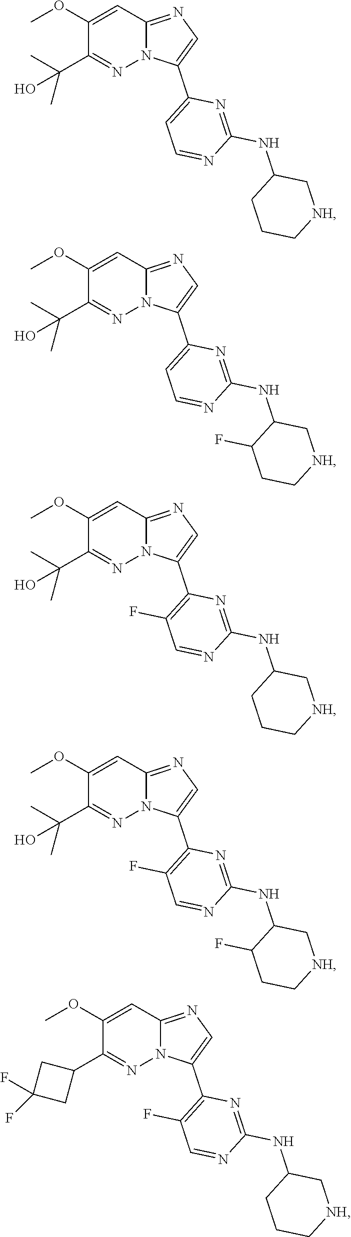 [SMILES: COc1cc2ncc(-c3ccnc(NC4CCCNC4)n3)n2nc1C(C)(C)O ; COc1cc2ncc(-c3ccnc(NC4CNCCC4F)n3)n2nc1C(C)(C)O ; COc1cc2ncc(-c3nc(NC4CCCNC4)ncc3F)n2nc1C(C)(C)O ; COc1cc2ncc(-c3nc(NC4CNCCC4F)ncc3F)n2nc1C(C)(C)O ; COc1cc2ncc(-c3nc(NC4CCCNC4)ncc3F)n2nc1C1CC(F)(F)C1]

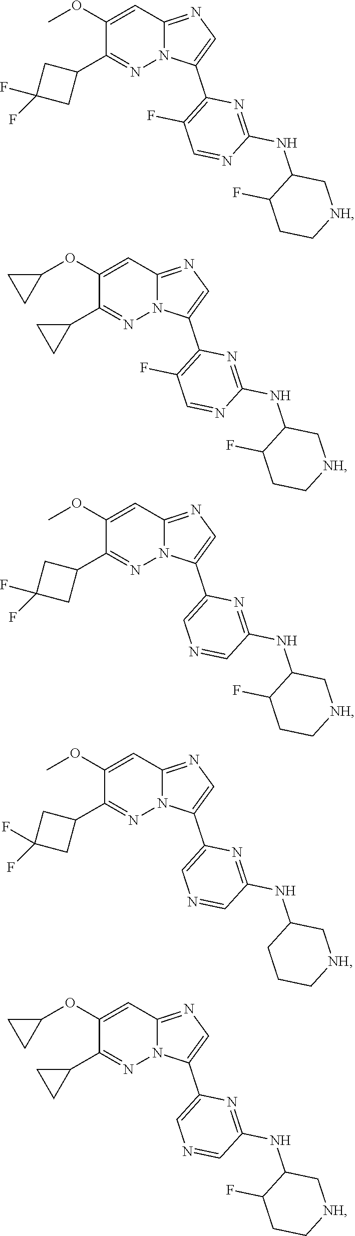 [SMILES: COc1cc2ncc(-c3nc(NC4CNCCC4F)ncc3F)n2nc1C1CC(F)(F)C1 ; Fc1cnc(NC2CNCCC2F)nc1-c1cnc2cc(OC3CC3)c(C3CC3)nn12 ; COc1cc2ncc(-c3cncc(NC4CNCCC4F)n3)n2nc1C1CC(F)(F)C1 ; COc1cc2ncc(-c3cncc(NC4CCCNC4)n3)n2nc1C1CC(F)(F)C1 ; FC1CCNCC1Nc1cncc(-c2cnc3cc(OC4CC4)c(C4CC4)nn23)n1]

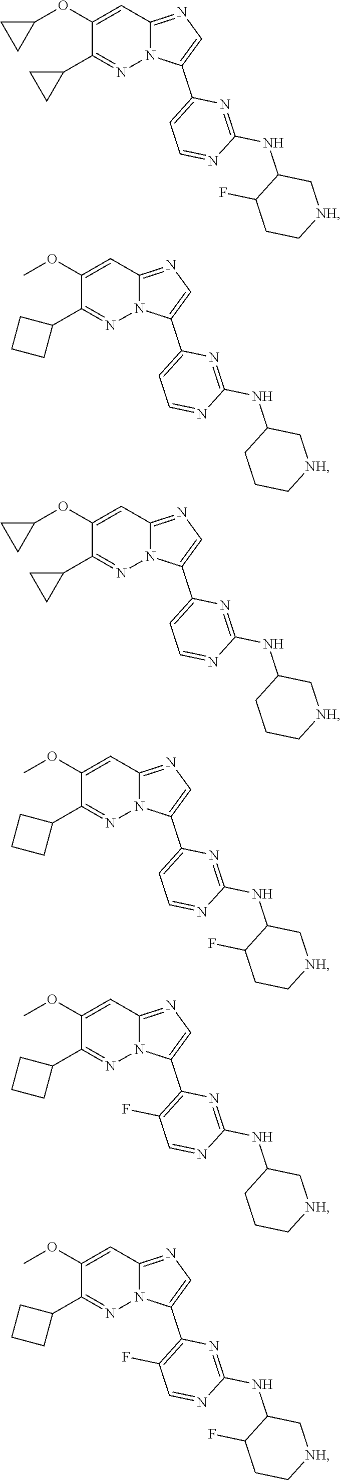 [SMILES: FC1CCNCC1Nc1nccc(-c2cnc3cc(OC4CC4)c(C4CC4)nn23)n1 ; COc1cc2ncc(-c3ccnc(NC4CCCNC4)n3)n2nc1C1CCC1 ; c1cc(-c2cnc3cc(OC4CC4)c(C4CC4)nn23)nc(NC2CCCNC2)n1 ; COc1cc2ncc(-c3ccnc(NC4CNCCC4F)n3)n2nc1C1CCC1 ; COc1cc2ncc(-c3nc(NC4CCCNC4)ncc3F)n2nc1C1CCC1 ; COc1cc2ncc(-c3nc(NC4CNCCC4F)ncc3F)n2nc1C1CCC1]

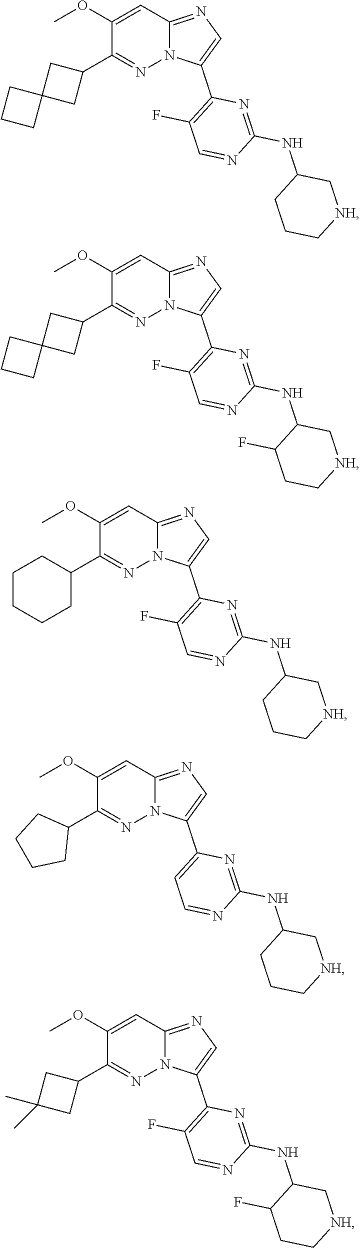 [SMILES: COc1cc2ncc(-c3nc(NC4CCCNC4)ncc3F)n2nc1C1CC2(CCC2)C1 ; COc1cc2ncc(-c3nc(NC4CNCCC4F)ncc3F)n2nc1C1CC2(CCC2)C1 ; COc1cc2ncc(-c3nc(NC4CCCNC4)ncc3F)n2nc1C1CCCCC1 ; COc1cc2ncc(-c3ccnc(NC4CCCNC4)n3)n2nc1C1CCCC1 ; COc1cc2ncc(-c3nc(NC4CNCCC4F)ncc3F)n2nc1C1CC(C)(C)C1]

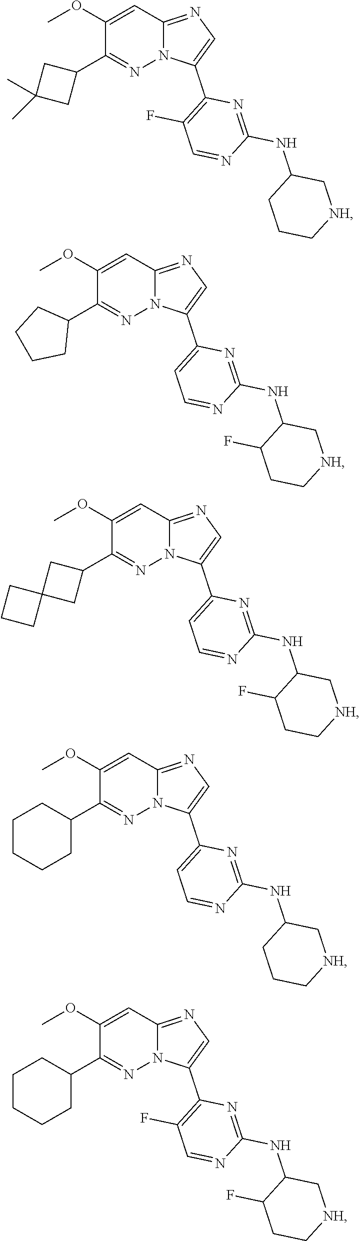 [SMILES: COc1cc2ncc(-c3nc(NC4CCCNC4)ncc3F)n2nc1C1CC(C)(C)C1 ; COc1cc2ncc(-c3ccnc(NC4CNCCC4F)n3)n2nc1C1CCCC1 ; COc1cc2ncc(-c3ccnc(NC4CNCCC4F)n3)n2nc1C1CC2(CCC2)C1 ; COc1cc2ncc(-c3ccnc(NC4CCCNC4)n3)n2nc1C1CCCCC1 ; COc1cc2ncc(-c3nc(NC4CNCCC4F)ncc3F)n2nc1C1CCCCC1]

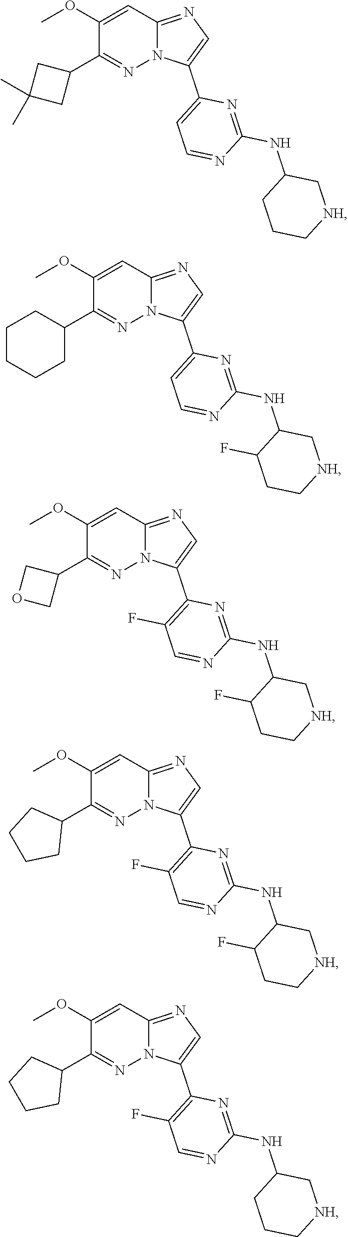 [SMILES: COc1cc2ncc(-c3ccnc(NC4CCCNC4)n3)n2nc1C1CC(C)(C)C1 ; COc1cc2ncc(-c3ccnc(NC4CNCCC4F)n3)n2nc1C1CCCCC1 ; COc1cc2ncc(-c3nc(NC4CNCCC4F)ncc3F)n2nc1C1COC1 ; COc1cc2ncc(-c3nc(NC4CNCCC4F)ncc3F)n2nc1C1CCCC1 ; COc1cc2ncc(-c3nc(NC4CCCNC4)ncc3F)n2nc1C1CCCC1]

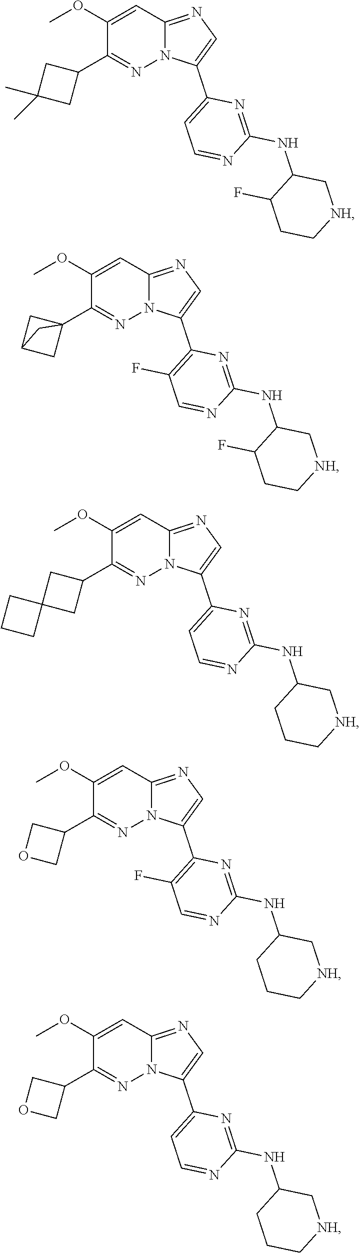 [SMILES: COc1cc2ncc(-c3ccnc(NC4CNCCC4F)n3)n2nc1C1CC(C)(C)C1 ; COc1cc2ncc(-c3nc(NC4CNCCC4F)ncc3F)n2nc1C12CC(C1)C2 ; COc1cc2ncc(-c3ccnc(NC4CCCNC4)n3)n2nc1C1CC2(CCC2)C1 ; COc1cc2ncc(-c3nc(NC4CCCNC4)ncc3F)n2nc1C1COC1 ; COc1cc2ncc(-c3ccnc(NC4CCCNC4)n3)n2nc1C1COC1]

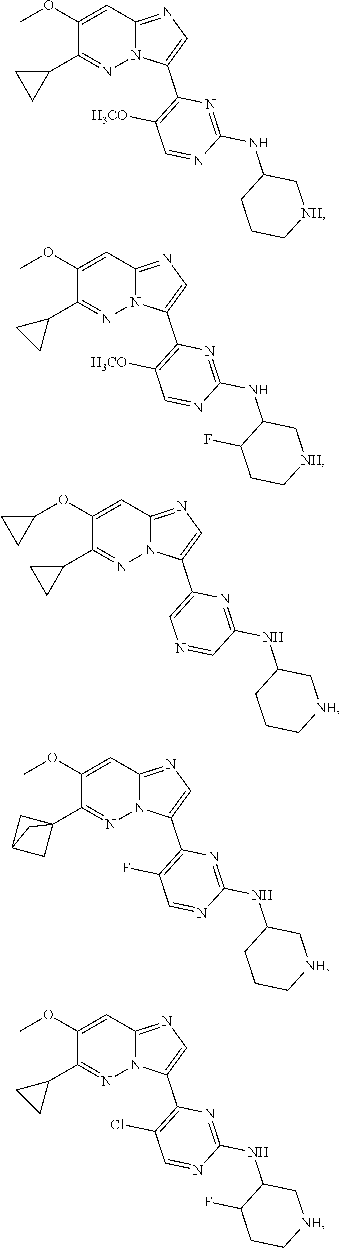 [SMILES: COc1cnc(NC2CCCNC2)nc1-c1cnc2cc(OC)c(C3CC3)nn12 ; COc1cnc(NC2CNCCC2F)nc1-c1cnc2cc(OC)c(C3CC3)nn12 ; c1ncc(-c2cnc3cc(OC4CC4)c(C4CC4)nn23)nc1NC1CCCNC1 ; COc1cc2ncc(-c3nc(NC4CCCNC4)ncc3F)n2nc1C12CC(C1)C2 ; COc1cc2ncc(-c3nc(NC4CNCCC4F)ncc3Cl)n2nc1C1CC1]

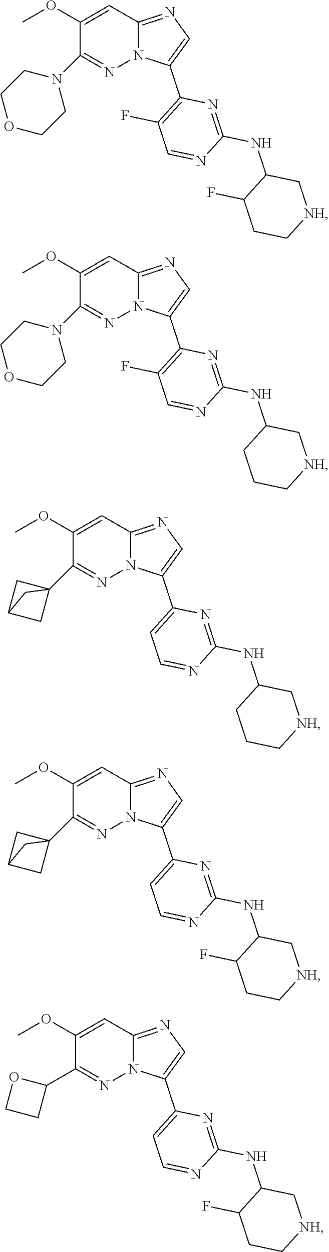 [SMILES: COc1cc2ncc(-c3nc(NC4CNCCC4F)ncc3F)n2nc1N1CCOCC1 ; COc1cc2ncc(-c3nc(NC4CCCNC4)ncc3F)n2nc1N1CCOCC1 ; COc1cc2ncc(-c3ccnc(NC4CCCNC4)n3)n2nc1C12CC(C1)C2 ; COc1cc2ncc(-c3ccnc(NC4CNCCC4F)n3)n2nc1C12CC(C1)C2 ; COc1cc2ncc(-c3ccnc(NC4CNCCC4F)n3)n2nc1C1CCO1]

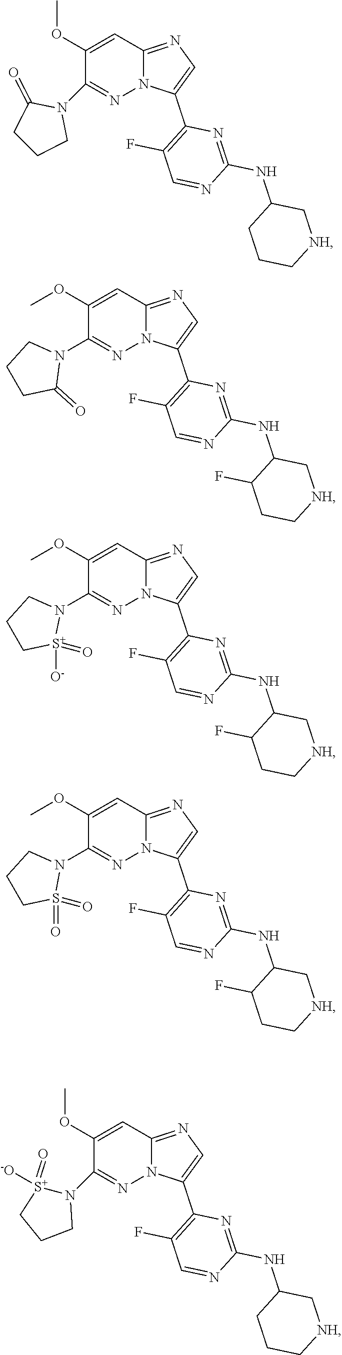

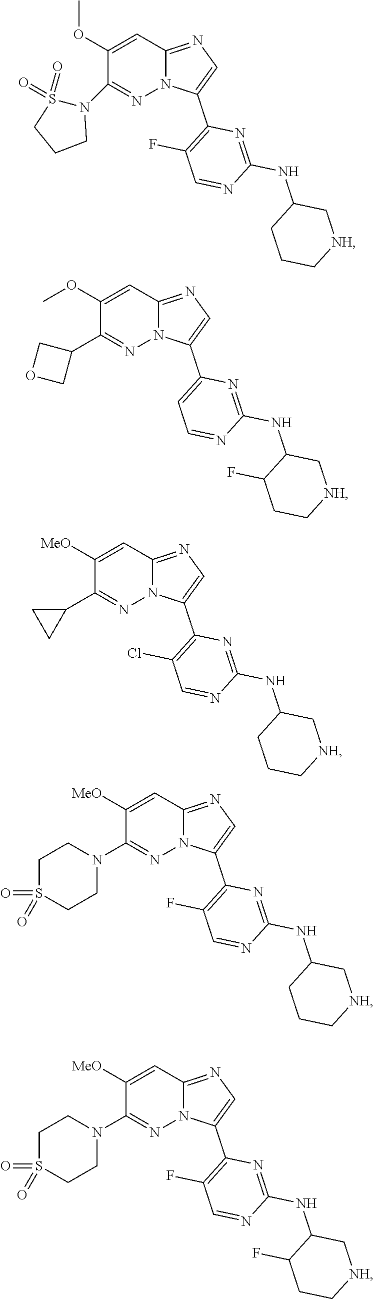 [SMILES: COc1cc2ncc(-c3nc(NC4CCCNC4)ncc3F)n2nc1N1CCCS1(=O)=O ; COc1cc2ncc(-c3ccnc(NC4CNCCC4F)n3)n2nc1C1COC1 ; COc1cc2ncc(-c3nc(NC4CCCNC4)ncc3Cl)n2nc1C1CC1 ; COc1cc2ncc(-c3nc(NC4CCCNC4)ncc3F)n2nc1N1CCS(=O)(=O)CC1 ; COc1cc2ncc(-c3nc(NC4CNCCC4F)ncc3F)n2nc1N1CCS(=O)(=O)CC1]

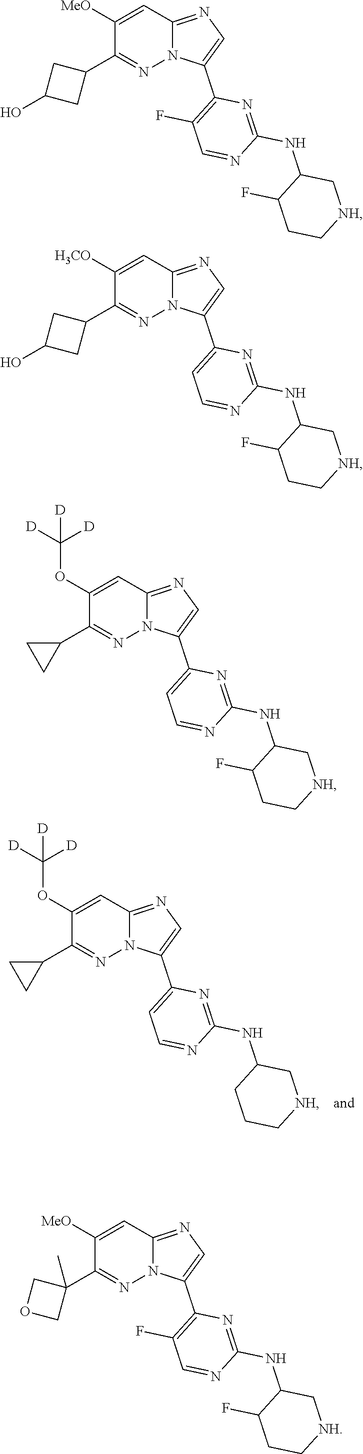 [SMILES: COc1cc2ncc(-c3nc(NC4CNCCC4F)ncc3F)n2nc1C1CC(O)C1 ; COc1cc2ncc(-c3ccnc(NC4CNCCC4F)n3)n2nc1C1CC(O)C1 ; [2H]C([2H])([2H])Oc1cc2ncc(-c3ccnc(NC4CNCCC4F)n3)n2nc1C1CC1 ; [2H]C([2H])([2H])Oc1cc2ncc(-c3ccnc(NC4CCCNC4)n3)n2nc1C1CC1 ; COc1cc2ncc(-c3nc(NC4CNCCC4F)ncc3F)n2nc1C1(C)COC1]

### Formula (IIIa-50100-WO)

In another embodiment, the compound of Formula (III) is a compound of Formula (IIa-5010-WO):

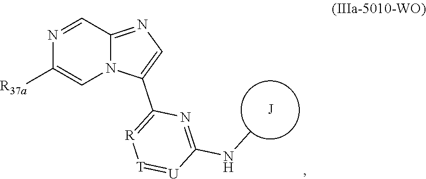

or a salt, ester, solvate, optical isomer, geometric isomer, or salt of an isomer thereof;
wherein:
   R is N or CR₃₁;
   Tis Nor CR₃₂;
   U is N or CR₃₃;

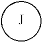

is

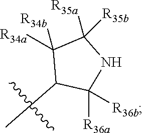

R₃₇ₐ is selected from halogen, C₁-C₆ alkyl, C₁-C₆ alkoxy, C₃-C₆ cycloalkyl, —O—(C₃-C₆ cycloalkyl), imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₃₁, R₃₂, and R₃₃ are each independently selected from H and halogen;
R₃₄ₐ, R_{34b}, R₃₅ₐ, R_{35b}, R₃₆ₐ, R_{36b}, R₃₈ₐ, and R_{38b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and one of R, T, or U is N.

In an embodiment, one or more of R₃₄ₐ, R_{34b}, R₃₅ₐ, R_{35b}, R₃₆ₐ, and R_{36b} in Formula (IIIa-5010-WO) is selected from halogen, —OH, optionally substituted C₁-C₆ alkyl, and optionally substituted C₁-C₆ alkoxy. In one embodiment, each of R₃₅ₐ, R_{35b}, R₃₆ₐ, and R_{36b} is H and R₃₄ₐ and/or R_{34b} is halogen. In one embodiment, each of R₃₅ₐ, R_{35b}, R₃₆ₐ, and R_{36b} is H and R₃₄ₐ and/or R_{34b} is F. In one embodiment, each of R_{34b}, R₃₅ₐ, R_{35b}, R₃₆ₐ, and R_{36b} is H and R₃₄ₐ is F.

In an embodiment, R₃₇ₐ of Formula (IIIa-5010-WO) is C₁-C₆ alkyl substituted with one —OH and/or halogen. In one embodiment, R₃₇ₐ is

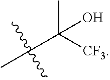 [SMILES: *C(C)(O)C(F)(F)F]

In an embodiment, R₃₇ₐ is 2-pyrrolidinonyl. In one embodiment, R₃₇ₐ is

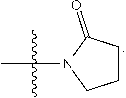 [SMILES: *N1CCCC1=O]

In an embodiment, the compound of Formula (IIIa-5010-WO) is a compound covered by the above formula and shown in Table 1 of the present application.

### Formula (IIIa-5010-WO50)

In another embodiment, the compound of Formula (III) is a compound of Formula (IIIa-5010-WO50):

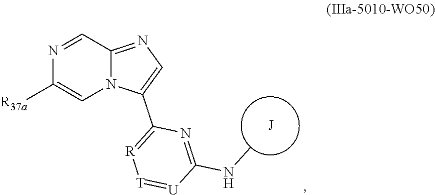

or a salt, ester, solvate, optical isomer, geometric isomer, or salt of an isomer thereof;
wherein:
   R is N or CR₃₁;
   T is N or CR₃₂;
   U is N or CR₃₃;

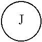

is

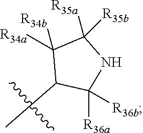

R₃₇ₐ is selected from halogen, C₁-C₆ alkyl, C₁-C₆ alkoxy, C₃-C₆ cycloalkyl, —O—(C₃-C₆ cycloalkyl), C₂-C₆ heterocyclyl, imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH, deuterium, and halogen, and wherein C₃-C₆ cycloalkyl, —O—(C₃-C₆ cycloalkyl), and C₂-C₆ heterocyclyl are each optionally substituted with one or more substituents selected from —OH, C₁-C₆ alkyl, and halogen;
R₃₁, R₃₂, and R₃₃ are each independently selected from H, C₁-C₆ alkoxy, and halogen; R₃₄ₐ, R_{34b}, R₃₅ₐ, R_{35b}, R₃₆ₐ, and R_{36b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms;
R₃₈ₐ and R_{38b} are each independently selected from H, C₁-C₆ alkyl, and C₃-C₆ cycloalkyl, wherein C₁-C₆ alkyl and C₃-C₆ cycloalkyl are each optionally substituted with one or more halogen; and
one of R, T, or U is N.

In one embodiment, R₃₁, R₃₂, and R₃₃ of Formula (IIIa-5010-WO50), if present, are H. In another embodiment, R is CR₃₁ wherein R₃₁ is F, T is CR₃₂ wherein R₃₂ is H, and U is N.

In an embodiment, one or more of R₃₄ₐ, R_{34b}, R₃₅ₐ, R_{35b}, R₃₆ₐ, and R_{36b} in Formula (IIIa-5010-WO50) is selected from halogen, —OH, optionally substituted C₁-C₆ alkyl, and optionally substituted C₁-C₆ alkoxy. In one embodiment, each of R₃₅ₐ, R_{35b}, R₃₆ₐ, and R_{36b} is H and R₃₄ₐ and/or R_{34b} is halogen. In one embodiment, each of R₃₅ₐ, R_{35b}, R₃₆ₐ, and R_{36b} is H and R₃₄ₐ and/or R_{34b} is F. In one embodiment, each of R_{34b}, R₃₅ₐ, R_{35b}, R₃₆ₐ, and R_{36b} is H and R₃₄ₐ is F.

In an embodiment, R₃₇ₐ of Formula (IIIa-5010-WO50) is C₁-C₆ alkyl substituted with one or more —OH and/or one or more halogen. In one embodiment, R₃₇ₐ is

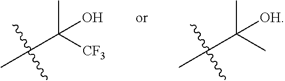

In one embodiment, the compound of Formula (III) is a compound of Formula (IIIa-5010-WO50), with the proviso that R₃₇ₐ is not

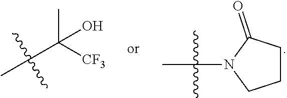

In an embodiment, the compound of Formula (IIIa-5010-WO50) is:

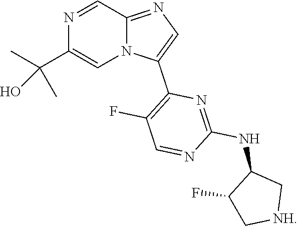 [SMILES: CC(C)(O)c1cn2c(-c3nc(N[C@H]4CNC[C@@H]4F)ncc3F)cnc2cn1]

In an embodiment, the compound of Formula (IIIa-5010-WO50) is:

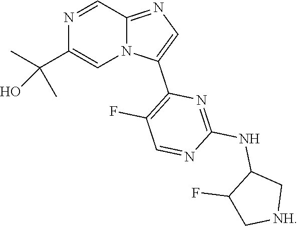 [SMILES: CC(C)(O)c1cn2c(-c3nc(NC4CNCC4F)ncc3F)cnc2cn1]

### Formula (IIb-5010-WO)

In another embodiment, the compound of Formula (III) is a compound of Formula (IIIb-5010-WO):

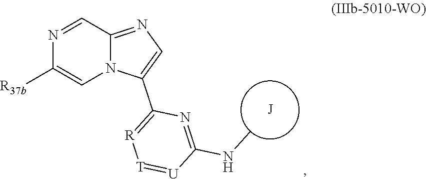

or a salt, ester, solvate, optical isomer, geometric isomer, or salt of an isomer thereof;
wherein:
   R is N or CR₃₁;
   T is N or CR₃₂;
   U is N or CR₃₃;

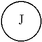

is

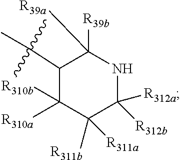

R_{37b} is selected from halogen, C₁-C₆ alkyl, C₁-C₆ alkoxy, C₃-C₆ cycloalkyl, —O—(C₃-C₆ cycloalkyl), imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₃₁, R₃₂, and R₃₃ are each independently selected from H and halogen;
R₃₈ₐ, R_{38b}, R₃₉ₐ, R_{39b}, R₃₁₀ₐ, R_{310b}, R₃₁₁ₐ, R_{311b}, R₃₁₂ₐ, and R_{312b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of R, T, or U is N.

In one embodiment, R₃₁, R₃₂, and R₃₃ of Formula (IIIb-5010-WO), if present, are H. In another embodiment, R is CR₃₁ wherein R₃₁ is F, T is CR₃₂ wherein R₃₂ is H, and U is N.

In an embodiment, each of R₃₉ₐ, R_{39b}, R₃₁₀ₐ, R_{310b}, R₃₁₁ₐ, R_{311b}, R₃₁₂ₐ, and R_{312b} in Formula (IIIb-5010-WO) is H. In another embodiment, one or more of R₃₉ₐ, R_{39b}, R₃₁₀ₐ, R_{310b}, R₃₁₁ₐ, R_{311b}, R₃₁₂ₐ, and R_{312b} is selected from halogen, —OH, optionally substituted C₁-C₆ alkyl, and optionally substituted C₁-C₆ alkoxy. In one embodiment, each of R₃₉ₐ, R_{39b}, R₃₁₁ₐ, R_{311b}, R₃₁₂ₐ, and R_{312b} is H and R₃₁₀ₐ and/or R_{310b} is halogen. In one embodiment, each of R₃₉ₐ, R_{39b}, R₃₁₁ₐ, R_{311b}, R₃₁₂ₐ, and R_{312b} is H and R₃₁₀ₐ and/or R_{310b} is F. In one embodiment, each of R₃₉ₐ, R_{39b}, R_{310b}, R₃₁₁ₐ, R_{311b}, R₃₁₂ₐ, and R_{312b} is H and R₃₁₀ₐ is F.

In an embodiment, R_{37b} of Formula (IIIb-5010-WO) is C₁-C₆ alkyl substituted with one —OH and/or halogen. In one embodiment, R_{37b} is

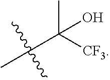 [SMILES: *C(C)(O)C(F)(F)F]

In an embodiment, R_{37b} is 2-pyrrolidinonyl. In one embodiment, R_{37b} is

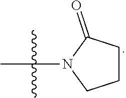 [SMILES: *N1CCCC1=O]

In an embodiment, the compound of Formula (IIIb-5010-WO) is a compound covered by the above formula and shown in Table 1 of the present application.

### Formula (IIb-5010-WO50)

In another embodiment, the compound of Formula (III) is a compound of Formula (IIIb-5010-WO50):

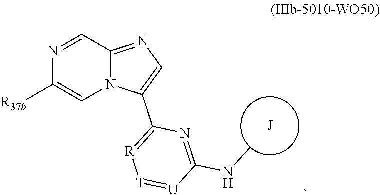

or a salt, ester, solvate, optical isomer, geometric isomer, or salt of an isomer thereof;
wherein:
   R is N or CR₃₁;
   T is N or CR₃₂;
   U is N or CR₃₃;

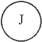

is

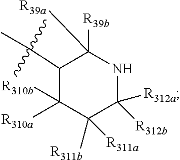

R_{37b} is selected from halogen, C₁-C₆ alkyl, C₁-C₆ alkoxy, C₃-C₆ cycloalkyl, —O—(C₃-C₆ cycloalkyl), C₂-C₆ heterocyclyl, imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH, deuterium, and halogen, and wherein C₃-C₆ cycloalkyl, —O—(C₃-C₆cycloalkyl), and C₂-C₆ heterocyclyl are each optionally substituted with one or more substituents selected from —OH, C₁-C₆ alkyl, and halogen;
R₃₁, R₃₂, and R₃₃ are each independently selected from H, C₁-C₆ alkoxy, and halogen;
R₃₉ₐ, R_{39b}, R₃₁₀ₐ, R_{310b}, R₃₁₁ₐ, R_{311b}, R₃₁₂ₐ, and R_{312b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms;
R₃₈ₐ and R_{38b} are each independently selected from H, C₁-C₆ alkyl, and C₃-C₆ cycloalkyl, wherein C₁-C₆ alkyl and C₃-C₆ cycloalkyl are each optionally substituted with one or more halogen; and
one of R, T, or U is N.

In one embodiment, R₃₁, R₃₂, and R₃₃ of Formula (IIIb-5010-WO50), if present, are H. In another embodiment, R is CR₃₁ wherein R₃₁ is F, T is CR₃₂ wherein R₃₂ is H, and U is N.

In an embodiment, each of R₃₉ₐ, R_{39b}, R₃₁₀ₐ, R_{310b}, R₃₁₁ₐ, R_{311b}, R₃₁₂ₐ, and R_{312b} in Formula (IIIb-5010-WO50) is H. In another embodiment, one or more of R₃₉ₐ, R_{39b}, R₃₁₀ₐ, R_{310b}, R₃₁₁ₐ, R_{311b}, R₃₁₂ₐ, and R_{312b} is selected from halogen, —OH, optionally substituted C₁-C₆ alkyl, and optionally substituted C₁-C₆ alkoxy. In one embodiment, each of R₃₉ₐ, R_{39b}, R₃₁₁ₐ, R_{311b}, R₃₁₂ₐ, and R_{312b} is H and R₃₁₀ₐ and/or R_{310b} is halogen. In one embodiment, each of R₃₉ₐ, R_{39b}, R₃₁₁ₐ, R_{311b}, R₃₁₂ₐ, and R_{312b} is H and R₃₁₀ₐ and/or R_{310b} is F. In one embodiment, each of R₃₉ₐ, R_{39b}, R_{310b}, R₃₁₁ₐ, R_{311b}, R₃₁₂ₐ, and R_{312b} is H and R₃₁₀ₐ is F.

In an embodiment, R_{37b} of Formula (IIIb-5010-WO50) is C₁-C₆ alkyl substituted with one —OH and/or halogen. In one embodiment, R_{37b} is

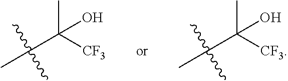

In an embodiment, R_{37b} is unsubstituted C₂-C₆ heterocyclyl. In one embodiment, R_{37b} is unsubstituted C₃ heterocyclyl. In one embodiment, R_{37b} is

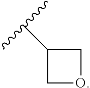 [SMILES: *C1COC1]

In one embodiment, the compound of Formula (III) is a compound of Formula (IIIb-5010-WO50) with the proviso that when R_{37b} is

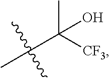 [SMILES: *C(C)(O)C(F)(F)F]

at least one of R₃₁, R₃₂, and R₃₃ is present and selected from C₁-C₆ alkoxy and halogen.

In one embodiment, the compound of Formula (IIIb-5010-WO50) is not:

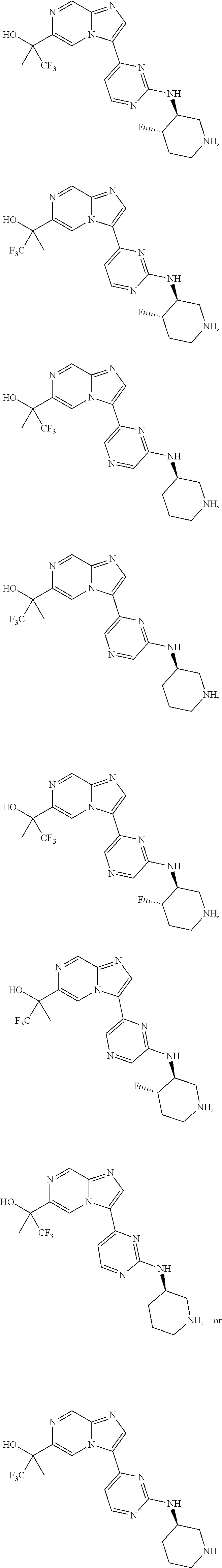

In an embodiment, the compound of Formula (IIIb-5010-WO50) is selected from:

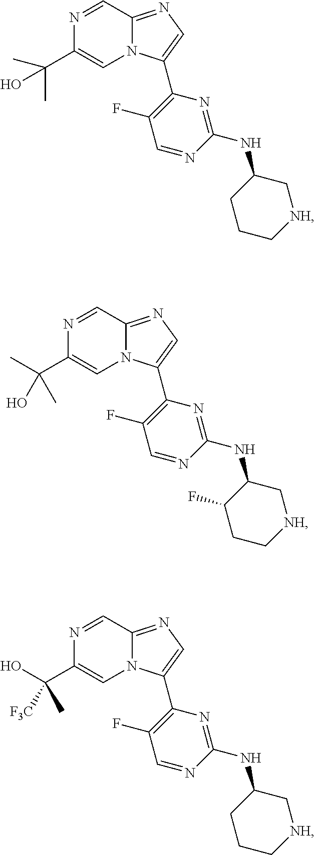 [SMILES: CC(C)(O)c1cn2c(-c3nc(N[C@@H]4CCCNC4)ncc3F)cnc2cn1 ; CC(C)(O)c1cn2c(-c3nc(N[C@H]4CNCC[C@@H]4F)ncc3F)cnc2cn1 ; C[C@@](O)(c1cn2c(-c3nc(N[C@@H]4CCCNC4)ncc3F)cnc2cn1)C(F)(F)F]

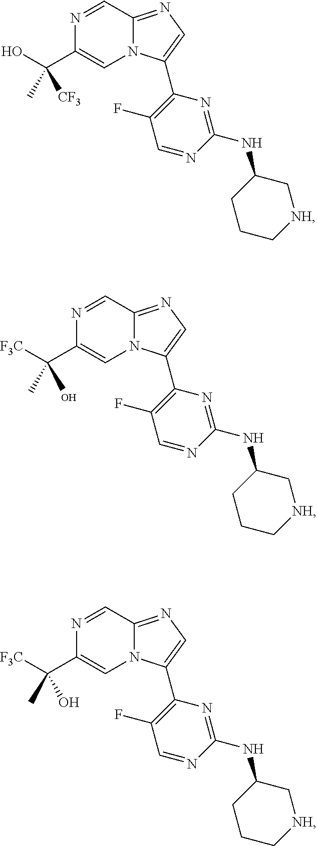 [SMILES: C[C@](O)(c1cn2c(-c3nc(N[C@@H]4CCCNC4)ncc3F)cnc2cn1)C(F)(F)F ; C[C@@](O)(c1cn2c(-c3nc(N[C@@H]4CCCNC4)ncc3F)cnc2cn1)C(F)(F)F ; C[C@](O)(c1cn2c(-c3nc(N[C@@H]4CCCNC4)ncc3F)cnc2cn1)C(F)(F)F]

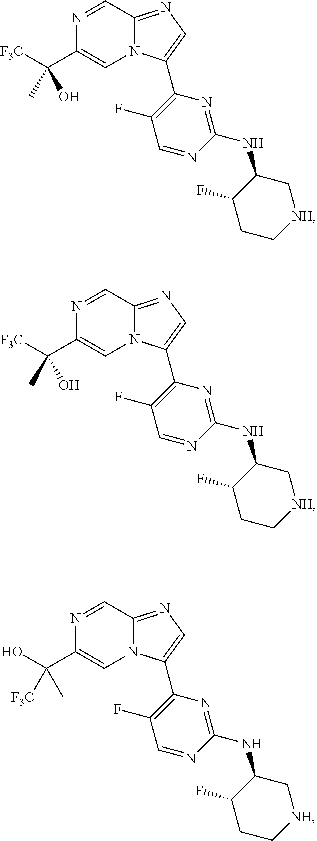 [SMILES: C[C@@](O)(c1cn2c(-c3nc(N[C@H]4CNCC[C@@H]4F)ncc3F)cnc2cn1)C(F)(F)F ; C[C@](O)(c1cn2c(-c3nc(N[C@H]4CNCC[C@@H]4F)ncc3F)cnc2cn1)C(F)(F)F ; CC(O)(c1cn2c(-c3nc(N[C@H]4CNCC[C@@H]4F)ncc3F)cnc2cn1)C(F)(F)F]

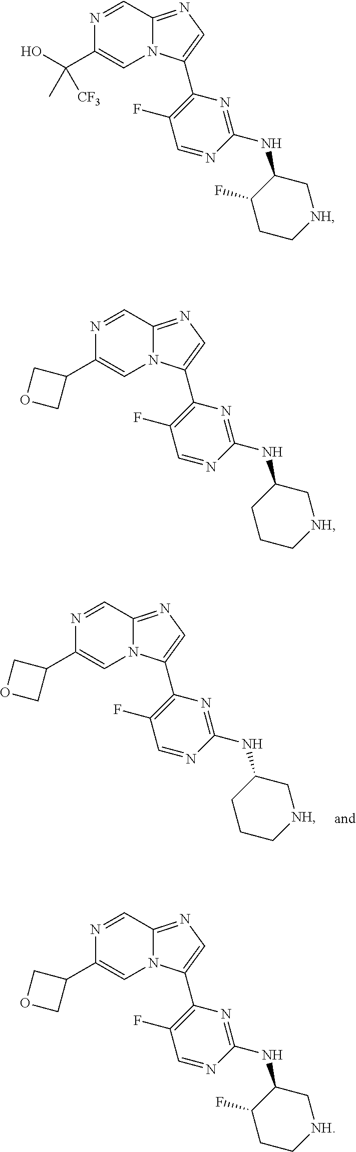 [SMILES: CC(O)(c1cn2c(-c3nc(N[C@H]4CNCC[C@@H]4F)ncc3F)cnc2cn1)C(F)(F)F ; Fc1cnc(N[C@@H]2CCCNC2)nc1-c1cnc2cnc(C3COC3)cn12 ; Fc1cnc(N[C@H]2CCCNC2)nc1-c1cnc2cnc(C3COC3)cn12 ; Fc1cnc(N[C@H]2CNCC[C@@H]2F)nc1-c1cnc2cnc(C3COC3)cn12]

In an embodiment, the compound of Formula (IIIb-5010-WO50) is selected from:

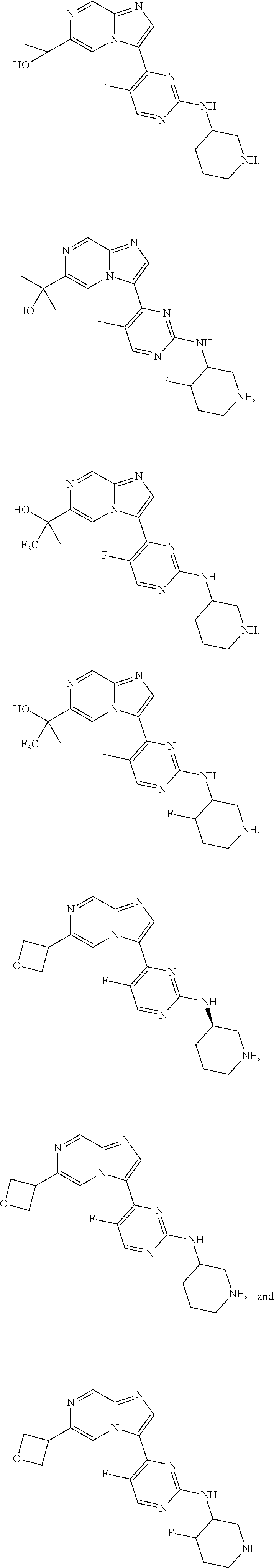

In some embodiments, the compounds described herein, can be in the form of salts, optical and geometric isomers, and salts of isomers. In other embodiments, the compounds can be in various forms, such as uncharged molecules, components of molecular complexes, or non-irritating pharmacologically acceptable salts, including but not limited to hydrochloride, hydrobromide, sulphate, phosphate, nitrate, borate, acetate, maleate, tartrate, and salicylate. In some instances, for acidic compounds, salts can include metals, amines, or organic cations (e.g. quaternary ammonium). In yet other embodiments, simple derivatives of the compounds (e.g., ethers, esters, or amides) which have desirable retention and release characteristics but which are easily hydrolyzed by body pH, enzymes, or other suitable means, can be employed.

In some embodiments, the compounds of the invention having a chiral center and can exist in and be isolated in optically active and racemic forms. In other embodiments, compounds may exhibit polymorphism. Some embodiments of the present invention encompass any racemic, optically active, polymorphic, or stereoisomeric form, or mixtures thereof, of a compound described herein, including isotopically-labeled and radio-labeled compounds. See e.g., Goding, 1986, Monoclonal Antibodies Principles and Practice; Academic Press, p. 104. Such isomers can be isolated by standard resolution techniques, including e.g., fractional crystallization, chiral chromatography, and the like. See e.g., Eliel, E. L. & Wilen S. H., 1993, Stereochemistry in Organic Compounds; John Wiley & Sons, New York. The preparation of optically active forms can be accomplished by any suitable method, including but not limited to, resolution of the racemic form by recrystallization techniques, synthesis from optically-active starting materials, chiral synthesis, or chromatographic separation using a chiral stationary phase.

In some embodiments, compounds disclosed herein have asymmetric centers and can occur as racemates, racemic mixtures, and as individual enantiomers or diastereoisomers, with all isomeric forms as well as mixtures thereof being contemplated for use in the compounds and methods described herein. The compounds contemplated for use in the compounds and methods described herein do not include those that are known in the art to be too unstable to synthesize and/or isolate.

The compounds disclosed herein can also contain unnatural proportions of atomic isotopes at one or more of the atoms that constitute such compounds. For example, the compounds can be radiolabeled with radioactive isotopes, such as for example tritium (³H), iodine-125 (¹²¹I), or carbon-14 (¹⁴C). All isotopic variations of the compounds disclosed herein, whether radioactive or not, are encompassed within the contemplated scope.

In some embodiments, metabolites of the compounds disclosed herein are useful for the methods disclosed herein.

In some embodiments, compounds contemplated herein may be provided in the form of a prodrug. The term “prodrug” refers to a compound that can be converted into a compound (e.g., a biologically active compound) described herein in vivo. Prodrugs can be useful for a variety of reason known in the art, including e.g., ease of administration due e.g., to enhanced bioavailability in oral administration, and the like. The prodrug can also have improved solubility in pharmaceutical compositions over the biologically active compounds. An example, without limitation, of a prodrug is a compound which is administered as an ester (i.e., the “prodrug”) to facilitate transmittal across a cell membrane where water solubility is detrimental to mobility but which then is metabolically hydrolyzed to the carboxylic acid, the active entity, once inside the cell where water solubility is beneficial. Conventional procedures for the selection and preparation of suitable prodrug derivatives are described, for example, in Design of Prodrugs, (ed. H. Bundgaard, Elsevier, 1985), which is hereby incorporated herein by reference for the limited purpose describing procedures and preparation of suitable prodrug derivatives.

Certain compounds disclosed herein can exist in unsolvated forms as well as solvated forms, including hydrated forms. In general, the solvated forms are equivalent to unsolvated forms and are encompassed within the scope of contemplated compounds. Certain compounds of the present invention can exist in multiple crystalline or amorphous forms. In general, all physical forms are equivalent for the compounds and methods contemplated herein and are intended to be within the scope disclosed herein.

In certain embodiments, one or more compounds of the invention (e.g., Formula (I), (II), (III), (V), (VI), (VII), (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)) can be part of a composition and can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, or no more than about 99.99%, from about 0.0001% to about 99%, from about 0.0001% to about 50%, from about 0.01% to about 95%, from about 1% to about 95%, from about 10% to about 90%, or from about 25% to about 75%.

In some embodiments, one or more compounds of the disclosure (e.g., Formula (I), (II), (III), (V), (VI), (VII), (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)) can be purified or isolated in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%.

### Methods for Preparing Compounds of Formula (I, (II), or (III)

In certain embodiments, a compound of Formula (I), (II), (III), (V), (VI), (VII), (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50) can be prepared comprising one or more of the steps set forth in Examples herein. The synthetic routes shown and described in Examples, can for example, be used to prepare Compounds herein, as set forth in Tables, and structurally related compounds.

### Pharmaceutical Compositions and Formulations

Some embodiments of the present invention include compositions comprising one or more compounds of the invention (e.g., Formula (I), (II), (III), (V), (VI), (VII), (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)). In certain embodiments, the composition is a pharmaceutical composition, such as compositions that are suitable for administration to animals (e.g., mammals, primates, monkeys, humans, canine, feline, porcine, mice, rabbits, rats, etc.). In some embodiments, there is provided a pharmaceutical composition comprising a compound disclosed herein and a pharmaceutically acceptable excipient. The compound can be a compound of any of Formulae (I), (II), (III), (V), (VI), (VII), (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50) as disclosed herein, a compound as set forth in Tables, or a pharmaceutically acceptable salt, ester, solvate, optical isomer, geometric isomer, salt of an isomer, prodrug, or derivative thereof. In some embodiments, the compound is set forth in any of Tables herein.

The term “pharmaceutically acceptable salts” is meant to include salts of the active compounds that are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galacturonic acids and the like (see, for example, Berge et al., “Pharmaceutical Salts”, Journal of Pharmaceutical Science, 1977, 66, 1-19). Certain specific compounds disclosed herein contain both basic and acidic functionalities that allow the compounds to be converted into either base or acid addition salts.

Compounds disclosed herein can exist as salts, such as with pharmaceutically acceptable acids. Accordingly, the compounds contemplated herein include such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts can be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Pharmaceutically acceptable salts of the compounds above, where a basic or acidic group is present in the structure, are also included within the scope of compounds contemplated herein. When an acidic substituent is present, such as —NHSO₃H, —COOH and —P(O)(OH)₂, there can be formed the ammonium, sodium, potassium, calcium salt, and the like, for use as the dosage form. Basic groups, such as amino or basic heteroaryl radicals, or pyridyl and acidic salts, such as hydrochloride, hydrobromide, acetate, maleate, palmoate, methanesulfonate, p-toluenesulfonate, and the like, can be used as the dosage form.

Also, in the embodiments in which R—COOH is present, pharmaceutically acceptable esters can be employed, e. g., methyl, ethyl, tert-butyl, pivaloyloxymethyl, and the like, and those esters known in the art for modifying solubility or hydrolysis characteristics for use as sustained release or prodrug formulations.

In some instances, the pharmaceutical composition is non-toxic, does not cause side effects, or both. In some embodiments, there may be inherent side effects (e.g., it may harm the patient or may be toxic or harmful to some degree in some patients).

In some embodiments, one or more compounds of the invention (e.g., Formula (I), (II), (III), (V), (VI), (VII), (la-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)) or can be part of a pharmaceutical composition and can be in an amount of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, the pharmaceutical composition can be presented in a dosage form which is suitable for the topical, subcutaneous, intrathecal, intraperitoneal, oral, parenteral, rectal, cutaneous, nasal, vaginal, or ocular administration route. In other embodiments, the pharmaceutical composition can be presented in a dosage form which is suitable for parenteral administration, a mucosal administration, intravenous administration, subcutaneous administration, topical administration, intradermal administration, oral administration, sublingual administration, intranasal administration, or intramuscular administration. The pharmaceutical composition can be in the form of, for example, tablets, capsules, pills, powders granulates, suspensions, emulsions, solutions, gels (including hydrogels), pastes, ointments, creams, plasters, drenches, delivery devices, suppositories, enemas, injectables, implants, sprays, aerosols or other suitable forms.

In some embodiments, the compounds disclosed herein can be administered orally as tablets, aqueous or oily suspensions, lozenges, troches, powders, granules, emulsions, capsules, syrups or elixirs. The composition for oral use can contain one or more agents selected from the group of sweetening agents, flavoring agents, coloring agents and preserving agents in order to produce pharmaceutically elegant and palatable preparations. Accordingly, there are also provided pharmaceutical compositions comprising a pharmaceutically acceptable carrier or excipient and one or more compounds disclosed herein.

In some embodiments, tablets contain the acting ingredient in admixture with non-toxic pharmaceutically acceptable excipients that are suitable for the manufacture of tablets. These excipients can be, for example, (1) inert diluents, such as calcium carbonate, lactose, calcium phosphate, carboxymethylcellulose, or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets can be uncoated or coated by known techniques to delay disintegration and absorption in the gastrointestinal tract and thereby provide a sustained action over a longer period. For example, a time delay material such as glyceryl monostearate or glyceryl distearate can be employed.

For preparing pharmaceutical compositions from the compounds disclosed herein, pharmaceutically acceptable carriers can be either solid or liquid. Solid form preparations include powders, tablets, pills, capsules, cachets, suppositories, and dispersible granules. A solid carrier can be one or more substance that can also act as diluents, flavoring agents, binders, preservatives, tablet disintegrating agents, or an encapsulating material.

A compound disclosed herein, in the form of a free compound or a pharmaceutically-acceptable pro-drug, metabolite, analogue, derivative, solvate or salt, can be administered, for in vivo application, parenterally by injection or by gradual perfusion over time. Administration can be intravenously, intraperitoneally, intramuscularly, subcutaneously, intracavity, or transdermally. For in vitro studies the compounds can be added or dissolved in an appropriate biologically acceptable buffer and added to a cell or tissue.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds disclosed herein are injectable, sterile solutions, preferably oily or aqueous solutions, as well as suspensions, emulsions, or implants, including suppositories. This suspension can be formulated according to known methods using those suitable dispersing or wetting agents and suspending agents that have been mentioned above. The sterile injectable preparation can also a sterile injectable solution or suspension in a non-toxic parenterally-acceptable diluent or solvent, for example, as a solution in 1,3-butanediol. Among the acceptable vehicles, carriers, and solvents that can be employed are water, Ringer's solution, and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds disclosed herein can also be incorporated into liposomes or administered via transdermal pumps or patches. Pharmaceutical admixtures suitable for use in the pharmaceuticals compositions and methods disclosed herein include those described, for example, in PHARMACEUTICAL SCIENCES (17th Ed., Mack Pub. Co., Easton, PA) and WO 96/05309, the teachings of both of which are hereby incorporated by reference.

In some embodiments, preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, growth factors and inert gases and the like.

Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV, 14th ed. Washington: American Pharmaceutical Association (1975), the contents of which are hereby incorporated by reference. The pH and exact concentration of the various components of the pharmaceutical composition are adjusted according to routine skills in the art. See e.g., Goodman and Gilman (eds.), 1990, THE PHARMACOLOGICAL BASIS FOR THERAPEUTICS (7th ed.).

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents. Aqueous suspensions normally contain the active materials in admixture with excipients suitable for the manufacture of aqueous suspension. Such excipients can be (1) suspending agent such as sodium carboxymethyl cellulose, methyl cellulose, hydroxypropylmethylcellulose, sodium alginate, polyvinylpyrrolidone, gum tragacanth and gum acacia; (2) dispersing or wetting agents which can be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate

Also included are solid form preparations that are intended to be converted, shortly before use, to liquid form preparations for oral administration. Such liquid forms include solutions, suspensions, and emulsions. These preparations can contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

In some embodiments, the pharmaceutical composition can include one or more formulary ingredients. A “formulary ingredient” can be any suitable ingredient (e.g., suitable for the drug(s), for the dosage of the drug(s), for the timing of release of the drugs(s), for the disease, for the disease state, or for the delivery route) including, but not limited to, water (e.g., boiled water, distilled water, filtered water, pyrogen-free water, or water with chloroform), sugar (e.g., sucrose, glucose, mannitol, sorbitol, xylitol, or syrups made therefrom), ethanol, glycerol, glycols (e.g., propylene glycol), acetone, ethers, DMSO, surfactants (e.g., anionic surfactants, cationic surfactants, zwitterionic surfactants, or nonionic surfactants (e.g., polysorbates)), oils (e.g., animal oils, plant oils (e.g., coconut oil or arachis oil), or mineral oils), oil derivatives (e.g., ethyl oleate, glyceryl monostearate, or hydrogenated glycerides), excipients, preservatives (e.g., cysteine, methionine, antioxidants (e.g., vitamins (e.g., A, E, or C), selenium, retinyl palmitate, sodium citrate, citric acid, chloroform, or parabens, (e.g., methyl paraben or propyl paraben)), or combinations thereof.

In certain embodiments, pharmaceutical compositions can be formulated to release the active ingredient (e.g., one or more compounds of the invention such as Formula (I)) substantially immediately upon the administration or any substantially predetermined time or time after administration. Such formulations can include, for example, controlled release formulations such as various controlled release compositions and coatings.

Other formulations (e.g., formulations of a pharmaceutical composition) can, in certain embodiments, include those incorporating the drug (or control release formulation) into food, food stuffs, feed, or drink.

Some compounds can have limited solubility in water and therefore can require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions can be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions disclosed herein can additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

There are provided various pharmaceutical compositions useful for ameliorating certain diseases and disorders. The pharmaceutical compositions according to one embodiment are prepared by formulating a compound disclosed herein in the form of a free compound or a pharmaceutically-acceptable pro-drug, metabolite, analogue, derivative, solvate or salt, either alone or together with other pharmaceutical agents, suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers.

There are provided various pharmaceutical compositions useful for ameliorating certain diseases and disorders. The pharmaceutical compositions according to one embodiment are prepared by formulating a compound disclosed herein in the form of a free compound or a pharmaceutically-acceptable pro-drug, metabolite, analogue, derivative, solvate or salt, either alone or together with other pharmaceutical agents, suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers.

### Methods of Treating and Preventing Disease

In addition to their ability to inhibit IRAK, IRAK inhibitors have been demonstrated to have selectivity for multiple kinases. In some embodiments, compounds described herein have inhibitory action against one or more kinases, such as interleukin-1 receptor-associated kinase (IRAK) and FMS-like tyrosine kinase 3 (FLT3). The inhibitory action against one or more kinases, such as IRAK and FLT3, can allow for treatment and/or prevention of diseases in an animal (e.g., mammals, porcine, canine, avian (e.g., chicken), bovine, feline, primates, rodents, monkeys, rabbits, mice, rats, and humans) using a compound of the invention (e.g., Formula (I)) including, but not limited to hematopoietic cancers (e.g., disorders of hematopoietic stem cells in the bone marrow or disorders related to myeloid lineage), MDS, AML, myeloproliferative disease, and diseases (e.g., hematopoietic cancers) related to mutations in IRAK1, IRAK4, and/or FLT3 (e.g., mutations in the juxtamembrane region of FLT3, mutations in the kinase domain of FLT3, FLT3 point mutations, FLT3 internal tandem duplication mutations, the FLT3-ITD mutation, the D835Y FLT3 mutation, the D835V FLT3 mutation, the F691L FLT3 mutation, or the R834Q FLT3 mutation).

In some embodiments, the compounds of the invention can inhibit the activity of one or more of FLT3, mutations of FLT3 (e.g., mutations in the juxtamembrane region of FLT3, mutations in the kinase domain of FLT3, FLT3 point mutations, FLT3 internal tandem duplication mutations, the FLT3-ITD mutation, the D835Y FLT3 mutation, the D835V FLT3 mutation, the F691L FLT3 mutation, or the R834Q FLT3 mutation), IRAK4 (interleukin-1 receptor associated kinase 4), isoforms of IRAK4, mutations of IRAK4, IRAK1 (interleukin-1 receptor associated kinase 1), isoforms of IRAK1, and/or mutations of IRAK1. In some embodiments, the compounds of the invention can inhibit the activity of one or both of FLT3 and mutations of FLT3 (e.g., mutations in the juxtamembrane region of FLT3, mutations in the kinase domain of FLT3, FLT3 point mutations, FLT3 internal tandem duplication mutations, the FLT3-ITD mutation, the D835Y FLT3 mutation, the D835V FLT3 mutation, the F691L FLT3 mutation, or the R834Q FLT3 mutation) and optionally inhibits one or more of IRAK4, isoforms of IRAK4, mutations of IRAK4, IRAK1, isoforms of IRAK1, or mutations of IRAK1. In some embodiments, the compounds of the invention can inhibit the activity of one or both of FLT3 and mutations of FLT3 (e.g., mutations in the juxtamembrane region of FLT3, mutations in the kinase domain of FLT3, FLT3 point mutations, FLT3 internal tandem duplication mutations, the FLT3-ITD mutation, the D835Y FLT3 mutation, the D835V FLT3 mutation, the F691L FLT3 mutation, or the R834Q FLT3 mutation) and optionally inhibits one or both of IRAK4 and IRAK1, or an isoform or mutation thereof. In some embodiments, the compounds of the invention can inhibit FLT3 in combination with IRAK4, IRAK1, or both IRAK4 and IRAK1. In one embodiment, the compounds of the invention are pan-FLT3 inhibitors. In one embodiment, the compounds of the invention are IRAK1, IRAK4, and pan-FLT3 inhibitors.

In some embodiments, compounds exhibit inhibitory activity against IRAK and/or FLT-3 with activities ≥1 μM, e.g., about 1, 2, 3, 4, 5, 6, 7, 8, 9, 10, 11, 12, 13, 14, 16, 18, 20, 22, 24, 26, 28, 30, 32, 34, 36, 38, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000 nM, or even greater. In some embodiments, the compounds exhibit inhibitory activity against IRAK and/or FLT-3 with activities between 0.1 nM and 1 nM, e.g., about 0.1, 0.2, 0.3, 0.4, 0.5, 0.6, 0.7, 0.8, 0.9 or 1.0 nM. In some embodiments, compounds described herein exhibit inhibitory activity against IRAK and/or FLT-3 with activities ≤0.1 μM, e.g., about 1, 2, 5, 10, 15, 20, 30, 40, 50, 60, 70, 80, 90, or 100 nM. Ranges of values using a combination of any of the values recited herein as upper and/or lower limits are also contemplated, for example, but not limited to, 1-10 nM, 10-100 nM, 1-100 nM, 0.1-1 nM, 0.1-100 nM, 0.1-200 nM, 1-200 nM, 10-200 nM, 100-200 nM, 200-500 nM, 0.1-500 nM, 1-500 nM, 10-500 nM, 500-1000 nM, 0.1-1000 nM, 1-1000 nM, 10-1000 nM, or 100-1000 nM. In some embodiments, the inhibitory activity is less than 0.1 nM, less than 1 nM, less than 10 nM, less than 100 nM, or less than 1000 nM. In some embodiments, the inhibitory activity is in the range of about 1-10 nM, 10-100 nM, 0.1-1 μM, 1-10 μM, 10-100 M, 100-200 μM, 200-500 μM, or even 500-1000 μM. It is understood that for purposes of quantification, the terms “activity,” “inhibitory activity,” “biological activity,” “IRAK activity,” “IRAK1 activity,” “IRAK4 activity,” “FLT-3 activity,” and the like in the context of an inhibitory compound disclosed herein can be quantified in a variety of ways known in the art. Unless indicated otherwise, as used herein such terms refer to IC₅₀ in the customary sense (i.e., concentration to achieve half-maximal inhibition. It is understood that for purposes of quantification, the terms “activity,” “inhibitory activity,” “biological activity,” “IRAK activity,” “IRAK1 activity,” “IRAK4 activity,” “FLT-3 activity,” and the like in the context of an inhibitory compound disclosed herein can be quantified in a variety of ways known in the art. Unless indicated otherwise, as used herein such terms refer to IC₅₀ in the customary sense (i.e., concentration to achieve half-maximal inhibition.

In some embodiments, hematopoietic cancers that can be treated in an animal (e.g., mammals, porcine, canine, avian (e.g., chicken), bovine, feline, primates, rodents, monkeys, rabbits, mice, rats, and humans) using a compound of the invention (e.g., Formula (I)) include, but are not limited to hematopoietic cancers and cancers of the myeloid line of blood cells, cancers with an increased risk of occurrence due to other blood disorders, cancers with an increased risk of occurrence due to chemical exposure (e.g., anti-cancer therapies or occupational chemical exposure), cancers with an increased risk of occurrence due to ionizing radiation (e.g., anti-cancer therapies), cancers evolving from myelodysplastic syndromes, cancers evolving from myeloproliferative disease, and cancers of the B cells.

In some embodiments, hematopoietic cancers that can be treated include, but are not limited to, MDS, AML, lymphoma, leukemia, bone marrow cancer, non-Hodgkin lymphoma, Waldenström's macroglobulinemia, B cell lymphoma, diffuse large B-cell lymphoma (DLBCL) (e.g. ABC DLBCL with MYD88 mutation (e.g., L265P)), follicular lymphoma, or marginal zone lymphoma, or combinations thereof.

In some embodiments, cancers characterized by dysregulated IRAK expression (IRAK1 and/or IRAK4) and/or IRAK-mediated intracellular signaling, can be treated, and include, but are not limited to, glioblastoma multiforme, endometrial cancer, melanoma, prostate cancer, lung cancer, breast cancer, kidney cancer, bladder cancer, basal cell carcinoma, thyroid cancer, squamous cell carcinoma, neuroblastoma, ovarian cancer, renal cell carcinoma, hepatocellular carcinoma, colon cancer, pancreatic cancer, rhabdomyosarcoma, meningioma, gastric cancer, Glioma, oral cancer, nasopharyngeal carcinoma, rectal cancer, stomach cancer, and uterine cancer, and the like, and combinations thereof.

In some embodiments, compounds of the present invention can be used to inhibit targets in the context of additional conditions characterized by over active IRAK1 and/or IRAK4. According to particular aspects of the invention, compounds of the present invention can be used to inhibit over active IRAK1 and/or IRAK4 in conditions such as inflammatory diseases and autoimmune disease, wherein said inflammatory diseases and autoimmune diseases are characterized by over active IRAK1 and/or IRAK4. In some embodiments, inflammatory and autoimmune diseases characterized by dysregulated (e.g., hyperactive) IRAK expression (IRAK1 and/or IRAK4) and/or IRAK-mediated intracellular signaling, can be treated, and include, but are not limited to, chronic inflammation (i.e., associated with viral and bacterial infection), sepsis, rheumatoid arthritis, systemic lupus erythematosus, inflammatory bowel disease, multiple sclerosis, psoriasis, Sjögren's syndrome, Ankylosing spondylitis, systemic sclerosis, Type 1 diabetes mellitus, and the like, and combinations thereof.

In certain embodiments, MDS that can be treated in a subject (e.g., mammals, porcine, canine, avian (e.g., chicken), bovine, feline, primates, rodents, monkeys, rabbits, mice, rats, and humans) using a compound of the invention (e.g., Formula (I)) include but are not limited to MDS with a splicing factor mutation, MDS with a mutation in isocitrate dehydrogenase 1, MDS with a mutation in isocitrate dehydrogenase 2, refractory cytopenia with unilineage dysplasia (e.g., refractory anemia, refractory neutropenia, and refractory thrombocytopenia), refractory anemia with ring sideroblasts, refractory cytopenia with multilineage dysplasia (e.g., refractory cytopenia with multilineage dysplasia and ring sideroblasts and animals with pathological changes not restricted to red cells such as prominent white cell precursor and platelet precursor (megakaryocyte) dysplasia), refractory anemias with excess blasts I and II, 5q-syndrome, megakaryocyte dysplasia with fibrosis, and refractory cytopenia of childhood. In some embodiments, MDS that can be treated include, but are not limited to, MDS that is inherited, MDS with an increased risk of occurrence due to an inherited predisposition, MDS with an increased risk of occurrence due to other blood disorders, MDS with an increased risk of occurrence due to chemical exposure, MDS with an increased risk of occurrence due to ionizing radiation, MDS with an increased risk of occurrence due to cancer treatment (e.g., a combination of radiation and the radiomimetic alkylating agents such as busulfan, nitrosourea, or procarbazine (with a latent period of 5 to 7 years) or DNA topoisomerase inhibitors), MDS evolving from acquired aplastic anemia following immunosuppressive treatment and Fanconi's anemia, MDS with an increased risk due to an mutation in splicing factors, MDS with an increased risk due to a mutation in isocitrate dehydrogenase 1, and MDS with an increased risk due to a mutation in isocitrate dehydrogenase 2. Animals that can be treated include but are not limited to mammals, rodents, primates, monkeys (e.g., macaque, rhesus macaque, pig tail macaque), humans, canine, feline, porcine, avian (e.g., chicken), bovine, mice, rabbits, and rats. In the methods, the term “subject” may refer to both human and non-human subjects. In some instances, the subject is in need of the treatment (e.g., by showing signs of disease or MDS, or by having a low blood cell count).

In some embodiments, MDS that can be treated in a subject (e.g., mammals, porcine, canine, avian (e.g., chicken), bovine, feline, primates, rodents, monkeys, rabbits, mice, rats, and humans) using a compound of the invention (e.g., Formula (I)) include, but are not limited to MDS that can be treated by inhibiting one or more of FLT3 (e.g., using FLT3 inhibitors), mutations of FLT3 (e.g., using inhibitors of FLT3 mutants), IRAK4 (e.g., using IRAK4 inhibitors), mutations of IRAK4 (e.g., using inhibitors of IRAK4 mutants), IRAK1 (e.g., using IRAK 1 inhibitors), and/or mutations of IRAK1 (e.g., using inhibitors of IRAK1 mutant). In certain embodiments, MDS that can be treated include, but are not limited to MDS that can be treated by inhibiting IRAK4 (or its mutations), MDS that can be treated by inhibiting and IRAK1 (or its mutations), or MDS that can be treated by inhibiting IRAK4 (or its mutations) and IRAK1 (or its mutations). In some embodiments, MDS that can be treated include, but are not limited to MDS that can be treated by inhibiting FLT3 in combination with IRAK4, IRAK1, or both IRAK4 and IRAK1. In some embodiments, inhibiting FLT3 in combination with IRAK4, IRAK1, or both IRAK4 and IRAK1 provides for treating tumors with FLT3 mutations, which can be or become resistant to FLT3 inhibitors due to adaptive resistance mechanism(s), e.g., driven by IRAK. In some embodiments, MDS that can be treated is characterized by MDS having enhanced IRAK4-Long expression and/or activity relative to IRAK4-Short, and/or wherein the MDS is not driven by FLT3 mutations but expresses IRAK4-Long, based on the use of IRAK4L and the ratio of IRAK4L to IRAK4S (e.g. as described in U.S. patent application Ser. No. 16/339,692; and Smith, M. A., et al. (2019). “U2AF1 mutations induce oncogenic IRAK4 isoforms and activate innate immune pathways in myeloid malignancies.” Nat Cell Biol 21(5):640-650. DOI: 10.1038/s41556-019-0314-5, both incorporated by reference herein in their entirety).

In some embodiments, AML that can be treated in a subject (e.g., mammals, porcine, canine, avian (e.g., chicken), bovine, feline, primates, rodents, monkeys, rabbits, mice, rats, and humans) using a compound of the invention (e.g., Formula (I)) include, but are not limited to AML that is inherited, AML with an increased risk of occurrence due to an inherited predisposition, AML with one or more recurrent genetic abnormality (e.g., with inversions or translocations, such as MLLT3/MLL which is a translocation between chromosome 9 and 11 (“MLL”) AML with translocation between chromosomes 8 and 21, AML with translocation or inversion in chromosome 16, AML with translocation between chromosomes 9 and 11, APL (M3) with translocation between chromosomes 15 and 17, AML with translocation between chromosomes 6 and 9, AML with translocation or inversion in chromosome 3, and the like), AML (megakaryoblastic) with a translocation between chromosomes 1 and 22, AML with myelodysplasia-related changes, AML related to previous chemotherapy or radiation (such as, for example, alkylating agent-related AML, topoisomerase II inhibitor-related AML, and the like), AML not otherwise categorized (does not fall into above categories—similar to FAB classification; such as, for example, AML minimally differentiated (M0), AML with minimal maturation (M1), AML with maturation (M2), acute myelomonocytic leukemia (M4), acute monocytic leukemia (M5), acute erythroid leukemia (M6), acute megakaryoblastic leukemia (M7), acute basophilic leukemia, acute panmyelosis with fibrosis, and the like), myeloid sarcoma (also known as granulocytic sarcoma, chloroma or extramedullary myeloblastoma), undifferentiated and biphenotypic acute leukemias (also known as mixed phenotype acute leukemias), AML with an increased risk of occurrence due to other blood disorders, AML with an increased risk of occurrence due to chemical exposure, AML with an increased risk of occurrence due to ionizing radiation, AML evolving from myelodysplastic syndromes, AML evolving from myeloproliferative disease, AML with an increased risk due to an FLT3 mutation, AML with an increased risk due to an FLT3 mutation in the juxtamembrane region of FLT3, AML with an increased risk due to an FLT3 mutation of an internal tandem duplication in the juxtamembrane region of FLT3, AML with an increased risk due to an FLT3 mutation in the kinase domain of FLT3, AML with an increased risk due to the FLT3 mutation D835Y, AML with an increased risk due to the FLT3 mutation D835V, AML with an increased risk due to the FLT3 mutation F691L, and AML with an increased risk due to the FLT3 mutation R834Q, and the like. In some embodiments, AML that can be treated include AML that by inhibiting one or more of FLT3 (e.g., using FLT3 inhibitors), mutations of FLT3 (e.g., using inhibitors of FLT3 mutants), IRAK4 (e.g., using IRAK4 inhibitors), mutations of IRAK4 (e.g., using inhibitors of IRAK4 mutants), IRAK1 (e.g., using IRAK 1 inhibitors), and/or mutations of IRAK1 (e.g., using inhibitors of IRAK1 mutant). In certain embodiments, AML that can be treated include, but are not limited to AML that can be treated by inhibiting IRAK4 (or its mutations), MDS that can be treated by inhibiting and IRAK1 (or its mutations), or AML that can be treated by inhibiting IRAK4 (or its mutations) and IRAK1 (or its mutations). In some embodiments, AML that can be treated include, but are not limited to AML that can be treated by inhibiting FLT3 in combination with IRAK4, IRAK1, or both IRAK4 and IRAK1. In some embodiments, inhibiting FLT3 in combination with IRAK4, IRAK1, or both IRAK4 and IRAK1 provides for treating tumors with FLT3 mutations which can be or become resistant to FLT3 inhibitors due to adaptive resistance mechanism(s), e.g. driven by IRAK. In some embodiments, AML that can be treated is characterized by AML having enhanced IRAK4-Long expression and/or activity relative to IRAK4-Short, and/or wherein the AML is not driven by FLT3 mutations but expresses IRAK4-Long, based on the use of IRAK4L and the ratio of IRAK4L to IRAK4S (e.g. as described in U.S. patent application Ser. No. 16/339,692; and Smith, M. A., et al. (2019). “U2AF1 mutations induce oncogenic IRAK4 isoforms and activate innate immune pathways in myeloid malignancies.” Nat Cell Biol 21(5): 640-650. DOI: 10.1038/s41556-019-0314-5, both incorporated by reference herein in their entirety).

In some embodiments, hematopoietic cancers that can be treated in a subject (e.g., mammals, porcine, canine, avian (e.g., chicken), bovine, feline, primates, rodents, monkeys, rabbits, mice, rats, and humans) using a compound of the invention (e.g., Formula (I)) include, but are not limited to hematopoietic cancers (e.g. MDS, AML, DLBCL, and the like, as described previously) that can be treated by inhibiting (e.g., reducing the activity or expression of) one or more of FLT3 (e.g., using FLT3 inhibitors), mutations of FLT3 (e.g., using inhibitors of FLT3 mutants), IRAK4 (e.g., using IRAK4 inhibitors), isoforms of IRAK4, mutations of IRAK4 (e.g., using inhibitors of IRAK4 mutants), IRAK1 (e.g., using IRAK 1 inhibitors), isoforms of IRAK1, or mutations of IRAK1 (e.g., using inhibitors of IRAK1 mutants). In certain embodiments, hematopoietic cancers that can be treated include, but are not limited to cancers that can be treated by inhibiting (e.g., reducing the activity or expression of) FLT3 (or its mutations) and IRAK4 (or its mutations), hematopoietic cancers that can be treated by inhibiting (e.g., reducing the activity or expression of) FLT3 (or its mutations) and IRAK1 (or its mutations), or hematopoietic cancers that can be treated by inhibiting (e.g., reducing the activity or expression of) FLT3 (or its mutations), IRAK4 (or its isoforms or mutations), and IRAK1 (or its isoforms or mutations). In some embodiments, hematopoietic cancer that can be treated include, but are not limited to hematopoietic cancer that can be treated by inhibiting FLT3 in combination with IRAK4, IRAK1, or both IRAK4 and IRAK1. In some embodiments, inhibiting FLT3 in combination with IRAK4, IRAK1, or both IRAK4 and IRAK1 provides for treating tumors with FLT3 mutations which can be or become resistant to FLT3 inhibitors due to adaptive resistance mechanism(s), e.g. driven by IRAK. In some embodiments, hematopoietic cancer that can be treated is characterized by hematopoietic cancer having enhanced IRAK4-Long expression and/or activity relative to IRAK4-Short, and/or wherein the hematopoietic cancer is not driven by FLT3 mutations but expresses IRAK4-Long, based on the use of IRAK4L and the ratio of IRAK4L to IRAK4S (e.g. as described in U.S. patent application Ser. No. 16/339,692; and Smith, M. A., et al. (2019). “U2AF1 mutations induce oncogenic IRAK4 isoforms and activate innate immune pathways in myeloid malignancies.” Nat Cell Biol 21(5): 640-650. DOI: 10.1038/s41556-019-0314-5, both incorporated by reference herein in their entirety).

In some embodiments, cancers that can be treated include, but are not limited to, glioblastoma multiforme, endometrial cancer, melanoma, prostate cancer, lung cancer, breast cancer, kidney cancer, bladder cancer, basal cell carcinoma, thyroid cancer, squamous cell carcinoma, neuroblastoma, ovarian cancer, renal cell carcinoma, hepatocellular carcinoma, colon cancer, pancreatic cancer, rhabdomyosarcoma, meningioma, gastric cancer, Glioma, oral cancer, nasopharyngeal carcinoma, rectal cancer, stomach cancer, and uterine cancer, and the like, and combinations thereof, that can be treated by inhibiting FLT3 in combination with IRAK4, IRAK1, or both IRAK4 and IRAK1. In some embodiments, inhibiting FLT3 in combination with IRAK4, IRAK1, or both IRAK4 and IRAK1 provides for treating tumors with FLT3 mutations which can be or become resistant to FLT3 inhibitors due to adaptive resistance mechanism(s), e.g., driven by IRAK. In some embodiments, cancer that can be treated is characterized by cancer having enhanced IRAK4-Long expression and/or activity relative to IRAK4-Short, and/or wherein the cancer is not driven by FLT3 mutations but expresses IRAK4-Long, based on the use of IRAK4L and the ratio of IRAK4L to IRAK4S (e.g. as described in U.S. patent application Ser. No. 16/339,692; and Smith, M. A., et al. (2019). “U2AF1 mutations induce oncogenic IRAK4 isoforms and activate innate immune pathways in myeloid malignancies.” Nat Cell Biol 21(5): 640-650. DOI: 10.1038/s41556-019-0314-5, both incorporated by reference herein in their entirety).

In some embodiments, inflammatory and autoimmune diseases characterized by dysregulated (e.g., hyperactive) IRAK expression (IRAK1 and/or IRAK4) and/or IRAK-mediated intracellular signaling, that can be treated include, but are not limited to, chronic inflammation (i.e., associated with viral and bacterial infection), sepsis, rheumatoid arthritis, systemic lupus erythematosus, inflammatory bowel disease, multiple sclerosis, psoriasis, Sjögren's syndrome, Ankylosing spondylitis, systemic sclerosis, Type 1 diabetes mellitus, and the like, and combinations thereof, that can be treated by inhibiting FLT3 in combination with IRAK4, IRAK1, or both IRAK4 and IRAK1. In one embodiment, the inflammatory disease that can be treated is Crohn's disease or colitis. In one embodiment, the inflammatory disease that can be treated is atopic dermatitis. In some embodiments, inhibiting FLT3 in combination with IRAK4, IRAK1, or both IRAK4 and IRAK1 treats inflammatory and autoimmune diseases with FLT3 mutations which can be or become resistant to FLT3 inhibitors due to adaptive resistance mechanism(s), e.g., driven by IRAK. In some embodiments, inflammatory and autoimmune disease that can be treated is characterized by inflammatory and autoimmune disease having enhanced IRAK4-Long expression and/or activity relative to IRAK4-Short, and/or wherein the inflammatory and autoimmune disease is not driven by FLT3 mutations but expresses IRAK4-Long, based on the use of IRAK4L and the ratio of IRAK4L to IRAK4S (e.g. as described in U.S. patent application Ser. No. 16/339,692; and Smith, M. A., et al. (2019). “U2AF1 mutations induce oncogenic IRAK4 isoforms and activate innate immune pathways in myeloid malignancies.” Nat Cell Biol 21(5): 640-650. DOI: 10.1038/s41556-019-0314-5, both incorporated by reference herein in their entirety).

As related to treating MDS (e.g., MDS with a splicing factor mutation, MDS with a mutation in isocitrate dehydrogenase 1, or MDS with a mutation in isocitrate dehydrogenase 2), treating can include but is not limited to prophylactic treatment and therapeutic treatment. As such, treatment can include, but is not limited to: preventing MDS (e.g., MDS with a splicing factor mutation, MDS with a mutation in isocitrate dehydrogenase 1, or MDS with a mutation in isocitrate dehydrogenase 2); reducing the risk of MDS (e.g., MDS with a splicing factor mutation, MDS with a mutation in isocitrate dehydrogenase 1, or MDS with a mutation in isocitrate dehydrogenase 2); ameliorating or relieving symptoms of MDS (e.g., MDS with a splicing factor mutation, MDS with a mutation in isocitrate dehydrogenase 1, or MDS with a mutation in isocitrate dehydrogenase 2); eliciting a bodily response against MDS (e.g., MDS with a splicing factor mutation, MDS with a mutation in isocitrate dehydrogenase 1, or MDS with a mutation in isocitrate dehydrogenase 2); inhibiting the development or progression of MDS (e.g., MDS with a splicing factor mutation, MDS with a mutation in isocitrate dehydrogenase 1, or MDS with a mutation in isocitrate dehydrogenase 2); inhibiting or preventing the onset of symptoms associated with MDS (e.g., MDS with a splicing factor mutation, MDS with a mutation in isocitrate dehydrogenase 1, or MDS with a mutation in isocitrate dehydrogenase 2); reducing the severity of MDS (e.g., MDS with a splicing factor mutation, MDS with a mutation in isocitrate dehydrogenase 1, or MDS with a mutation in isocitrate dehydrogenase 2); causing a regression of MDS (e.g., MDS with a splicing factor mutation, MDS with a mutation in isocitrate dehydrogenase 1, or MDS with a mutation in isocitrate dehydrogenase 2) or one or more of the symptoms associated with MDS (e.g., an increase in blood cell count); causing remission of MDS (e.g., MDS with a splicing factor mutation, MDS with a mutation in isocitrate dehydrogenase 1, or MDS with a mutation in isocitrate dehydrogenase 2); causing remission of MDS (e.g., MDS with a splicing factor mutation, MDS with a mutation in isocitrate dehydrogenase 1, or MDS with a mutation in isocitrate dehydrogenase 2) by preventing or minimizing FLT3 mutations (e.g., internal tandem duplication mutations or the D835Y mutation); preventing relapse of MDS (e.g., MDS with a splicing factor mutation, MDS with a mutation in isocitrate dehydrogenase 1, or MDS with a mutation in isocitrate dehydrogenase 2); or preventing relapse of MDS (e.g., MDS with a splicing factor mutation, MDS with a mutation in isocitrate dehydrogenase 1, or MDS with a mutation in isocitrate dehydrogenase 2) in animals or humans that have intrinsic or acquired resistance to other MDS treatments. In some embodiments, treating does not include prophylactic treatment of MDS (e.g., preventing or ameliorating future MDS).

As related to treating hematopoietic cancer (e.g., acute myeloid leukemia, lymphoma, leukemia, bone marrow cancer, non-Hodgkin lymphoma, or Waldenström's macroglobulinemia, B cell lymphoma, diffuse large B-cell lymphoma (DLBCL), DLBCL MYD88 mutation (e.g., ABC DLBCL with MYD88 mutation L265P), follicular lymphoma, or marginal zone lymphoma, and combinations thereof, and the like), treating can include but is not limited to prophylactic treatment and therapeutic treatment. As such, treatment can include, but is not limited to: preventing cancer (e.g., acute myeloid leukemia, lymphoma, leukemia, bone marrow cancer, non-Hodgkin lymphoma, or Waldenström's macroglobulinemia, B cell lymphoma, diffuse large B-cell lymphoma (DLBCL), DLBCL MYD88 mutation, follicular lymphoma, or marginal zone lymphoma, and combinations thereof, and the like); reducing the risk of cancer (e.g., acute myeloid leukemia, lymphoma, leukemia, bone marrow cancer, non-Hodgkin lymphoma, or Waldenström's macroglobulinemia, B cell lymphoma, diffuse large B-cell lymphoma (DLBCL), DLBCL MYD88 mutation, follicular lymphoma, or marginal zone lymphoma, and combinations thereof, and the like); ameliorating or relieving symptoms of cancer (e.g., acute myeloid leukemia, lymphoma, leukemia, bone marrow cancer, non-Hodgkin lymphoma, or Waldenström's macroglobulinemia, B cell lymphoma, diffuse large B-cell lymphoma (DLBCL), DLBCL MYD88 mutation, follicular lymphoma, or marginal zone lymphoma, and combinations thereof, and the like); eliciting a bodily response against cancer (e.g., acute myeloid leukemia, lymphoma, leukemia, bone marrow cancer, non-Hodgkin lymphoma, or Waldenström's macroglobulinemia, B cell lymphoma, diffuse large B-cell lymphoma (DLBCL), DLBCL MYD88 mutation, follicular lymphoma, or marginal zone lymphoma, and combinations thereof, and the like); inhibiting the development or progression of cancer (e.g., acute myeloid leukemia, lymphoma, leukemia, bone marrow cancer, non-Hodgkin lymphoma, or Waldenström's macroglobulinemia, B cell lymphoma, diffuse large B-cell lymphoma (DLBCL), DLBCL MYD88 mutation, follicular lymphoma, or marginal zone lymphoma, and combinations thereof, and the like); inhibiting or preventing the onset of symptoms associated with cancer (e.g., acute myeloid leukemia, lymphoma, leukemia, bone marrow cancer, non-Hodgkin lymphoma, or Waldenström's macroglobulinemia, B cell lymphoma, diffuse large B-cell lymphoma (DLBCL), DLBCL MYD88 mutation, follicular lymphoma, or marginal zone lymphoma, and combinations thereof, and the like); reducing the severity of cancer (e.g., acute myeloid leukemia, lymphoma, leukemia, bone marrow cancer, non-Hodgkin lymphoma, Waldenström's macroglobulinemia, B cell lymphoma, diffuse large B-cell lymphoma (DLBCL), DLBCL MYD88 mutation, follicular lymphoma, or marginal zone lymphoma, and combinations thereof, and the like); causing a regression of cancer (e.g., acute myeloid leukemia, lymphoma, leukemia, bone marrow cancer, non-Hodgkin lymphoma, Waldenström's macroglobulinemia, B cell lymphoma, diffuse large B-cell lymphoma (DLBCL), DLBCL MYD88 mutation, follicular lymphoma, or marginal zone lymphoma, and combinations thereof, and the like) or one or more of the symptoms associated with cancer (e.g., a decrease in tumor size); causing remission of cancer (e.g., acute myeloid leukemia, lymphoma, leukemia, bone marrow cancer, non-Hodgkin lymphoma, Waldenstrom's macroglobulinemia, B cell lymphoma, diffuse large B-cell lymphoma (DLBCL), DLBCL MYD88 mutation, follicular lymphoma, or marginal zone lymphoma, and combinations thereof, and the like); causing remission of cancer (e.g., acute myeloid leukemia, lymphoma, leukemia, bone marrow cancer, non-Hodgkin lymphoma, Waldenstrom's macroglobulinemia, B cell lymphoma, diffuse large B-cell lymphoma (DLBCL), DLBCL MYD88 mutation, follicular lymphoma, or marginal zone lymphoma, and combinations thereof, and the like) by preventing or minimizing FLT3 mutations (e.g., internal tandem duplication mutations or the D835Y mutation); causing remission of acute myeloid leukemia by preventing or minimizing FLT3 mutations (e.g., internal tandem duplication mutations or the D835Y mutation); preventing relapse of cancer (e.g., acute myeloid leukemia, lymphoma, leukemia, bone marrow cancer, non-Hodgkin lymphoma, Waldenstrom's macroglobulinemia, B cell lymphoma, diffuse large B-cell lymphoma (DLBCL), DLBCL MYD88 mutation, follicular lymphoma, or marginal zone lymphoma, and combinations thereof, and the like); preventing relapse of cancer (e.g., acute myeloid leukemia, lymphoma, leukemia, bone marrow cancer, non-Hodgkin lymphoma, Waldenstrom's macroglobulinemia, B cell lymphoma, diffuse large B-cell lymphoma (DLBCL), DLBCL MYD88 mutation, follicular lymphoma, or marginal zone lymphoma, and combinations thereof, and the like) in animals that have intrinsic or acquired resistance to other cancer treatments (e.g., from some FLT3 inhibitors or from MLL); or preventing relapse of acute myeloid leukemia in animals that have intrinsic or acquired resistance to other cancer treatments (e.g., from some FLT3 inhibitors or from MLL). In some embodiments, treating does not include prophylactic treatment of cancer (e.g., preventing or ameliorating future cancer).

Treatment of a subject can occur using any suitable administration method (such as those disclosed herein) and using any suitable amount of a compound of the invention (e.g., Formula (I)). In some embodiments, methods of treatment comprise treating an animal or human for MDS (e.g., MDS with a splicing factor mutation, MDS with a mutation in isocitrate dehydrogenase 1, or MDS with a mutation in isocitrate dehydrogenase 2). In some embodiments, methods of treatment comprise treating an animal or human for a hematopoietic cancer (e.g., acute myeloid leukemia, lymphoma, leukemia, bone marrow cancer, non-Hodgkin lymphoma, Waldenstrom's macroglobulinemia Waldenstrom's macroglobulinemia, B cell lymphoma, diffuse large B-cell lymphoma (DLBCL), DLBCL MYD88 mutation, follicular lymphoma, or marginal zone lymphoma, and combinations thereof, and the like). Other embodiments include treatment after one or more of having a blood disorder, having myelodysplastic syndrome, having myeloproliferative disease, an occurrence of chemical exposure, an exposure to ionizing radiation, or a treatment for a hematopoietic cancer (e.g., with chemotherapy, ionizing radiation, or both). Some embodiments of the invention include a method for treating a subject (e.g., an animal such as a human or primate) with a composition comprising a compound of the invention (e.g., Formula (I)) (e.g., a pharmaceutical composition) which comprises one or more administrations of one or more such compositions; the compositions may be the same or different if there is more than one administration.

In some embodiments, the method of treatment includes administering to a subject an effective amount of a composition comprising a compound of the invention (e.g., Formula (I), (II), (III), (V), (VI), (VII), (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)). As used herein, the term “effective amount” refers to a dosage or a series of dosages sufficient to affect treatment (e.g., to treat MDS such as but not limited to MDS (e.g., MDS with a splicing factor mutation, MDS with a mutation in isocitrate dehydrogenase 1, or MDS with a mutation in isocitrate dehydrogenase 2); or to treat a hematopoietic cancer, such as but not limited to acute myeloid leukemia, lymphoma, leukemia, bone marrow cancer, non-Hodgkin lymphoma, Waldenstrom's macroglobulinemia, B cell lymphoma, diffuse large B-cell lymphoma (DLBCL), DLBCL MYD88 mutation, follicular lymphoma, or marginal zone lymphoma, and combinations thereof, and the like) in a subject. In some embodiments, an effective amount can encompass a therapeutically effective amount, as disclosed herein. In certain embodiments, an effective amount can vary depending on the subject and the particular treatment being affected. The exact amount that is required can, for example, vary from subject to subject, depending on the age and general condition of the subject, the particular adjuvant being used (if applicable), administration protocol, and the like. As such, the effective amount can, for example, vary based on the particular circumstances, and an appropriate effective amount can be determined in a particular case. An effective amount can, for example, include any dosage or composition amount disclosed herein. In some embodiments, an effective amount of at least one compound of the invention (which can be administered to a subject such as mammals, primates, monkeys or humans) can be an amount of about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some embodiments, the dosage can be about 0.5 mg/kg body weight or about 6.5 mg/kg body weight. In some instances, an effective amount of at least one compound of the invention (e.g., Formula (I) such as but not limited to Compounds 1-77, 209-214, and 1a-115a, as listed in Tables 1, 6, and 49) (which can be administered to a subject such as mammals, rodents, mice, rabbits, feline, porcine, or canine) can be an amount of about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. In some embodiments, an effective amount of at least one compound of the invention (which can be administered to an animal such as mammals, primates, monkeys or humans) can be an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg. In regard to some conditions, the dosage can be about 20 mg/kg human body weight or about 100 mg/kg human body weight. In some instances, an effective amount of at least one compound of the invention (which can be administered to an animal such as mammals, rodents, mice, rabbits, feline, porcine, or canine) can be an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg.

In some embodiments, the treatments can also include one or more of surgical intervention, chemotherapy, radiation therapy, hormone therapies, immunotherapy, and adjuvant systematic therapies. Adjuvants may include but are not limited to chemotherapy (e.g., temozolomide), radiation therapy, antiangiogenic therapy (e.g., bevacizumab), and hormone therapies, such as administration of LHRH agonists; anti-estrogens, such as tamoxifen; high-dose progestogens; aromatase inhibitors, and/or adrenalectomy. Chemotherapy can be used as a single-agent or as a combination with known or new therapies.

In some embodiments, the administration to a subject of at least one compound of the invention (e.g., Formula (I)) is an adjuvant cancer therapy or part of an adjuvant cancer therapy. Adjuvant treatments include treatments by the mechanisms disclosed herein and of cancers as disclosed herein, including, but not limited to tumors. Corresponding primary therapies can include, but are not limited to, surgery, chemotherapy, or radiation therapy. In some instances, the adjuvant treatment can be a combination of chemokine receptor antagonists with traditional chemotoxic agents or with immunotherapy that increases the specificity of treatment to the cancer and potentially limits additional systemic side effects. In still other embodiments, a compound of the invention (e.g., Formula (I)) can be used as adjuvant with other chemotherapeutic agents. The use of a compound of the invention (e.g., Formula (I)) may, in some instances, reduce the duration of the dose of both drugs and drug combinations reducing the side effects.

In some embodiments, the administration to a subject may decrease the incidence of one or more symptoms associated with MDS/AML/a type of hematopoietic cancer. In some embodiments, the administration may decrease marrow failure, immune dysfunction, transformation to overt leukemia, or combinations thereof in said subject, as compared to a subject not receiving said composition.

In some embodiments, the method may decrease a marker of viability of MDS cells or cancer cells in a subject. In one aspect, the method may decrease a marker of viability of MDS, AML, and/or cancer cells. The marker may be selected from survival over time, proliferation, growth, migration, formation of colonies, chromatic assembly, DNA binding, RNA metabolism, cell migration, cell adhesion, inflammation, or a combination thereof.

### Combination Therapies

In one embodiment, the compounds of Formula (I) are administered with one or more therapeutic agents. Exemplary therapeutic agents include, but are not limited to, a CDK inhibitor, a BCL2 inhibitor, a PTEFb inhibitor, a DNA polymerase inhibitor, a cytidine deaminase inhibitor, a DNA methyltransferase (DNMT) inhibitor, an immunomodulatory imide, a cereblon modulator, a purine nucleoside antimetabolite, a Type II topoisomerase inhibitor, a DNA intercalator, a hedgehog antagonist, an IDH2 inhibitor, an IDH1 inhibitor, a ribonucleotide reductase inhibitor, an adenosine deaminase inhibitor, a Mek 1/2 inhibitor, an ERK 1/2 inhibitor, an AKT inhibitor, a PTPN11 inhibitor, an SHP2 inhibitor, a glucocorticoid steroid, a menin inhibitor, an MDM2 inhibitor, a BTK inhibitor, and a mutant/inactivated p53 reactivator.

In one embodiment, the therapeutic agent comprises a BCL2 inhibitor. In one embodiment, the BCL2 inhibitor is venetoclax or a salt thereof. In one embodiment, the therapeutic agent comprises a DNA polymerase inhibitor. In one embodiment, the DNA polymerase inhibitor is cytidine. In one embodiment, the therapeutic agent comprises a cytidine deaminase inhibitor. In one embodiment, the cytidine deaminase inhibitor is zebularine. In one embodiment, the therapeutic agent comprises a DNMT inhibitor. In one embodiment, the DNMT inhibitor is zebularine, decitabine, or 5-azacitidine. In one embodiment, the therapeutic agent comprises an immunomodulatory imide (cereblon modulator). In one embodiment, the immunomodulatory imide (cereblon modulator) is lenalidomide. In one embodiment, the therapeutic agent comprises a purine nucleoside antimetabolite. In one embodiment, the purine nucleoside antimetabolite is clofarabine. In one embodiment, the therapeutic agent comprises a Type II topoisomerase inhibitor/DNA intercalator. In one embodiment, the Type II topoisomerase inhibitor/DNA intercalator is vosaroxin. In one embodiment, the therapeutic agent comprises a hedgehog antagonist. In one embodiment, the hedgehog antagonist is glasdegib. In one embodiment, the therapeutic agent comprises an IDH1 inhibitor. In one embodiment, the IDH1 inhibitor is ivosidenib. In one embodiment, the therapeutic agent comprises an IDH2 inhibitor. In one embodiment, the IDH2 inhibitor is enasidenib. In one embodiment, the therapeutic agent comprises a ribonucleotide reductase inhibitor. In one embodiment, the ribonucleotide reductase inhibitor is gemcitabine. In one embodiment, the therapeutic agent comprises an adenosine deaminase inhibitor. In one embodiment, the adenosine deaminase inhibitor is cladribine. In one embodiment, the therapeutic agent comprises a Mek 1/2 inhibitor. In one embodiment, the Mek 1/2 inhibitor is trametinib. In one embodiment, the therapeutic agent comprises an ERK 1/2 inhibitor. In one embodiment, the ERK 1/2 inhibitor is ulixertinib. In one embodiment, the therapeutic agent comprises an AKT inhibitor. In one embodiment, the AKT inhibitor is capivasertib (AZD5363). In one embodiment, the therapeutic agent comprises a PTPN11/SHP2 inhibitor. In one embodiment, the PTPN11/SHP2 inhibitor is TNO-155. In one embodiment, the therapeutic agent comprises a glucocorticoid steroid. In one embodiment, the glucocorticoid steroid is prednisolone. In one embodiment, the therapeutic agent comprises a menin inhibitor. In one embodiment, the menin inhibitor is SNDX-5613. In one embodiment, the therapeutic agent comprises an MDM2 inhibitor. In one embodiment, the MDM2 inhibitor is navtemadlin (AMG 232, KRT-232). In one embodiment, the therapeutic agent comprises a BTK inhibitor. In one embodiment, the BTK inhibitor is selected from ibrutinib, acalabrutinib, and zanubrutinib. In one embodiment, the therapeutic agent comprises a mutant/inactivated p53 reactivator. In one embodiment, the mutant/inactivated p53 reactivator is Eprenetapopt (APR-246).

In one embodiment, the therapeutic agent comprises a CDK inhibitor. The CDK inhibitor can be any CDK inhibitor known to a person of ordinary skill in the art. In one embodiment, the CDK inhibitor is a CKD1, CKD2, CDK3, CDK4, CDK5, CDK6, CDK7, CDK8, CDK9, CDK10, CDK11, CDK12, or CDK13 inhibitor or a combination thereof.

In one embodiment, the CDK inhibitor comprises an inhibitor described in one of the following patents or patent applications: US 20210332071, US 20210330653, WO 2021214253, WO 2021178595, WO 2021207632, U.S. Pat. No. 8,685,660, US 20200361906, U.S. Ser. No. 10/695,346, U.S. Ser. No. 11/142,507, WO 2021198439, WO 2021201170, U.S. Pat. No. 8,153,632, U.S. Ser. No. 11/013,743, U.S. Ser. No. 11/135,198, US 20210299111, WO 2021190637, WO 2021188855, WO 2021188849, US 20210292299, U.S. Ser. No. 11/124,836, U.S. Ser. No. 10/961,527, US 20210284629, US 20210283265, WO 2021183994, WO 2021181233, U.S. Ser. No. 11/116,755, WO 2021176045, WO 2021177816, WO 2021176049, WO 2021176349, US 20210275522, US 20210275491, US 20210277037, U.S. Ser. No. 11/111,250, WO 2021142448, WO 2021172359, WO 2021174195, US 20210260209, US 20210261609, US 20210261636, US 20210261546, WO 2021168341, U.S. Ser. No. 11/014,911, U.S. Pat. Nos. 9,932,344, 8,415,355, U.S. Ser. No. 11/091,485, U.S. Ser. No. 11/091,490, US 20210246422, US 20210246138, US 20210244715, U.S. Ser. No. 11/083,722, U.S. Ser. No. 11/083,728, US 20210238226, US 20190142835, WO 2021155006, WO 2021152107, WO 2021155192, U.S. Ser. No. 10/294,234, U.S. Ser. No. 11/077,156, WO 2021148793, WO 2021149817, US 20210228529, US 20210228546, US 20210228723, U.S. Ser. No. 10/568,887, US 20190209549, U.S. Ser. No. 11/072,596, US 20210222133, U.S. Ser. No. 10/336,760, WO 2021144302, US 20210196796, U.S. Ser. No. 11/066,404, US 20210213029, US 20210213012, U.S. Pat. Nos. 9,642,835, 8,673,972, US 20210205304, WO 2021108648, WO 2020140054, U.S. Ser. No. 11/053,238, U.S. Ser. No. 11/052,087, U.S. Ser. No. 10/245,251, WO 2021133601, WO 2021133957, WO 2021127133, WO 2021124106, WO 2021122745, U.S. Ser. No. 10/849,903, US 20210186979, U.S. Ser. No. 11/040,957, WO 2021115335, U.S. Ser. No. 11/034,710, WO 2021110136, WO 2021110731, WO 2021108927, WO 2021110122, US 20210171554, U.S. Ser. No. 10/966,977, US 20210171498, U.S. Ser. No. 11/028,087, US 20210161909, WO 2021108581, U.S. Ser. No. 10/300,073, WO 2021102234, WO 2021102410, U.S. Ser. No. 11/014,906, U.S. Ser. No. 11/013,728, WO 2021092672, US 20210147424, US 20180147202, US 20210145974, U.S. Ser. No. 11/007,174, US 20210139459, US 20210139474, US 20210139436, US 20210139483, U.S. Ser. No. 10/221,140, US 20210128555, WO 2021087183, WO 2021084540, WO 2021087138, WO 2021087044, WO 2021079273, WO 2021053667, U.S. Ser. No. 10/729,692, WO 2021072475, WO 2021074338, WO 2021073593, U.S. Ser. No. 10/857,156, U.S. Ser. No. 10/870,651, U.S. Ser. No. 10/981,919, U.S. Ser. No. 10/202,392, WO 2021068867, WO 2021072017, WO 2021067569, US 20210093730, WO 2021067792, US 20210101881, U.S. Ser. No. 10/131,679, U.S. Ser. No. 10/730,887, WO 2021061695, WO 2021061752, WO 2021057867, WO 2021055705, WO 2021055014, WO 2021047573, US 20210070761, U.S. Ser. No. 10/946,012, WO 2021045586, WO 2021045585, WO 2021045582, WO 2021043190, U.S. Ser. No. 10/774,047, U.S. Ser. No. 10/941,126, U.S. Ser. No. 10/308,648, US 20210053969, U.S. Pat. No. 8,598,186, U.S. Ser. No. 10/927,113, US 20210047292, U.S. Ser. No. 10/829,490, WO 2021030843, WO 2021030620, WO 2021030623, U.S. Ser. No. 10/918,648, WO 2021023104, WO 2021026349, US 20210041441, U.S. Ser. No. 10/913,983, US 20210032596, WO 2021016663, U.S. Ser. No. 10/047,070, WO 2021014360, U.S. Ser. No. 10/899,742, WO 2021009701, WO 2021011796, WO 2021011864, WO 2021011802, US 20210015819, US 20210015817, US 20200129489, U.S. Ser. No. 10/273,252, U.S. Pat. No. 9,498,471, WO 2021003314, US 20200405809, U.S. Ser. No. 10/786,578, WO 2020263830, WO 2020259556, WO 2020263186, WO 2020259463, U.S. Ser. No. 10/835,531, WO 2020253458, WO 2020256637, WO 2020256868, WO 2020257615, US 20200397772, U.S. Ser. No. 10/871,495, US 20200392139, U.S. Ser. No. 10/730,870, U.S. Ser. No. 10/758,541, U.S. Ser. No. 10/799,506, US 20200347036, WO 2020245402, WO 2020244612, US 20200384027, US 20200383984, US 20200377904, WO 2020188100, WO 2020239558, WO 2020240360, WO 2020237025, US 20200369715, U.S. Ser. No. 10/844,021, US 20200361853, US 20200361943, WO 2020228513, U.S. Ser. No. 10/835,535, US 20200353107, WO 2020224568, WO 2020224609, WO 2020222668, WO 2020223609, US 20200347079, US 20200345736, US 20200345699, U.S. Ser. No. 10/702,527, US 20200339944, US 20200339615, US 20200339556, US 20200338209, WO 2020219650, WO 2020218518, WO 2020219926, WO 2019217581, US 20200331909, US 20200323851, WO 2020207260, WO 2020206583, U.S. Ser. No. 10/233,188, WO 2020205486, WO 2020202232, WO 2020206137, WO 2020206034, US 20200317693, US 20180280407, WO 2020193802, US 20200297704, U.S. Ser. No. 10/195,200, WO 2020191002, U.S. Ser. No. 10/780,179, WO 2020183307, US 20200289520, US 20200289506, U.S. Ser. No. 10/774,053, US 20200281918, U.S. Ser. No. 10/654,831, U.S. Ser. No. 10/479,785, WO 2020180706, U.S. Ser. No. 10/766,884, U.S. Ser. No. 10/767,162, WO 2020176794, WO 2020176510, WO 2020176505, U.S. Ser. No. 10/689,347, U.S. Ser. No. 10/736,902, U.S. Ser. No. 10/738,067, U.S. Pat. No. 9,913,844, US 20180135044, WO 2020159980, WO 2020160537, WO 2020160157, WO 2020157709, U.S. Ser. No. 10/618,905, US 20200237743, WO 2020152629, U.S. Ser. No. 10/722,505, U.S. Pat. No. 7,850,990, WO 2020148635, WO 2020092720, U.S. Pat. No. 9,845,331, U.S. Ser. No. 10/717,749, US 20200222478, WO 2020146355, US 20200216548, US 20200216450, WO 2020140052, WO 2020135507, WO 2020138370, WO 2020140055, WO 2020140098, WO 2020125513, WO 2020128878, WO 2020132259, US 20200197536, U.S. Ser. No. 10/132,797, US 20200190075, US 20200179451, US 20200181164, WO 2020108407, U.S. Pat. No. 9,746,457, U.S. Ser. No. 10/660,896, WO 2020099470, US 20200155519, US 20200155550, US 20200155526, U.S. Ser. No. 10/071,985, US 20200147089, US 20200147235, U.S. Ser. No. 10/555,931, WO 2020092528, WO 2020092621, US 20200131189, WO 2020053664, WO 2020091688, US 20200129473, U.S. Pat. No. 9,498,543, U.S. Ser. No. 10/633,374, US 20200123174, US 20200121803, US 20200108142, WO 2020070296, WO 2020060238, WO 2020058820, WO 2020058458, WO 2020052627, WO 2020052772, US 20200078362, U.S. Ser. No. 10/047,052, U.S. Ser. No. 10/317,405, U.S. Ser. No. 10/314,842, WO 2020041243, U.S. Ser. No. 10/570,141, US 20200054635, U.S. Ser. No. 10/383,873, U.S. Pat. No. 9,758,539, US 20200048228, US 20140309224, U.S. Ser. No. 10/519,136, WO 2020023917, WO 2020023768, US 20200016156, U.S. Ser. No. 10/532,103, US 20200010902, US 20200009134, WO 2020010158, US 20200000932, US 20200002432, US 20200002421, U.S. Ser. No. 10/513,507, WO 2019241636, WO 2019238088, WO 2019236901, US 20190374550, US 20190369104, WO 2019230654, U.S. Pat. No. 8,329,683, U.S. RE47,739, WO 2019222521, US 20190343961, U.S. Ser. No. 10/464,927, U.S. Ser. No. 10/463,690, WO 2019209825, U.S. Pat. No. 8,864,743, US 20150291562, WO 2019195959, US 20190310259, US 20190290637, US 20190292602, US 20170067116, U.S. Ser. No. 10/190,104, US 20160264552, US 20180135135, U.S. Ser. No. 10/413,552, WO 2019170055, US 20190275049, WO 2019168446, US 20190270967, US 20190248774, WO 2019159126, WO 2019150181, US 20180098963, US 20190224189, US 20160024084, WO 2019143719, WO 2019143730, U.S. Ser. No. 10/357,493, WO 2019138354, U.S. Ser. No. 10/351,578, U.S. Ser. No. 10/342,798, WO 2019129232, US 20190192522, U.S. Ser. No. 10/294,457, U.S. Ser. No. 10/323,035, US 20190175560, US 20190160021, U.S. Ser. No. 10/308,654, U.S. Ser. No. 10/308,602, WO 2019103050, WO 2019104065, US 20190153107, US 20190153108, US 20190151325, U.S. Pat. No. 9,617,225, U.S. Ser. No. 10/292,986, US 20190144876, U.S. Ser. No. 10/285,979, US 20190133980, WO 2019082124, US 20190125864, U.S. Ser. No. 10/273,240, WO 2019075011, US 20190105340, US 20190105309, WO 2019057141, U.S. Pat. No. 9,878,994, WO 2019054865, US 20190085375, US 20140271459, US 20190062340, WO 2019037742, U.S. Ser. No. 10/214,542, U.S. Ser. No. 10/214,492, WO 2019035904, WO 2019034147, U.S. Ser. No. 10/206,908, US 20160184311, U.S. Ser. No. 10/202,377, US 20190031650, WO 2019015690, WO 2019015689, US 20190022235, U.S. Ser. No. 10/179,770, WO 2019007321, US 20180370991, US 20180371021, U.S. Ser. No. 10/030,018, WO 2018228990, WO 2018218633, US 20180340148, U.S. Pat. No. 9,416,131, WO 2018013867, WO 2018202866, US 20160361314, US 20180298024, US 20180280392, US 20180271891, US 20180057497, WO 2018156812, WO 2018157069, US 20180221382, US 20180215731, U.S. Ser. No. 10/039,771, US 20180208989, US 20130035336, US 20180200279, WO 2018121766, US 20180179524, U.S. Ser. No. 10/005,836, US 20180170897, U.S. Pat. Nos. 9,982,045, 9,376,465, WO 2018089902, US 20180127748, U.S. Pat. Nos. 9,669,034, 6,933,315, WO 2018081211, WO 2018081204, U.S. Pat. Nos. 9,957,484, 9,957,273, 9,957,251, US 20180104330, WO 2018055492, U.S. Pat. No. 9,925,192, US 20160375024, WO 2018045956, US 20150322528, U.S. Pat. Nos. 9,907,753, 9,902,716, US 20180049997, U.S. Pat. Nos. 9,890,429, 9,884,849, US 20180028686, US 20180029985, U.S. Pat. No. 9,877,954, US 20180015153, US 20110086349, U.S. Pat. No. 9,862,717, US 20180000771, WO 2018001270, WO 2018005445, US 20170368069, WO 2017214335, WO 2017211245, U.S. Pat. Nos. 9,828,373, 9,827,309, 9,822,182, 9,814,714, US 20170312339, US 20170314077, WO 2017185662, U.S. Pat. Nos. 9,073,922, 9,790,189, 9,782,406, WO 2017164230, U.S. Pat. Nos. 8,742,205, 9,770,445, WO 2017160568, WO 2017149502, U.S. Pat. No. 9,745,325, WO 2017133701, WO 2017133542, U.S. Pat. No. 9,585,970, WO 2017130219, US 20170202893, U.S. Pat. No. 9,708,293, WO 2017114351, US 20140031302, US 20170174713, WO 2017100432, US 20170157212, WO 2017094026, U.S. Pat. Nos. 9,670,213, 9,670,161, US 20170152269, U.S. Pat. No. 9,611,313, US 20170128424, WO 2017060322, U.S. Pat. Nos. 9,650,358, 9,629,863, 9,498,532, US 20170106082, US 20170100569, U.S. Pat. No. 9,579,283, US 20170049899, US 20170037004, WO 2017012599, US 20170008904, US 20160368980, WO 2016192630, US 20160346334, U.S. Pat. Nos. 9,499,492, 9,241,941, WO 2016173557, U.S. Pat. No. 9,475,825, WO 2016123054, U.S. Pat. No. 9,458,106, WO 2016150902, US 20150148345, US 20160271156, WO 2016146591, US 20160256458, US 20160256448, WO 2016135046, U.S. Pat. Nos. 9,429,566, 9,422,307, 8,754,050, WO 2016127963, U.S. Pat. Nos. 9,415,118, 9,408,847, 9,408,848, WO 2016112177, U.S. Pat. Nos. 9,359,306, 8,623,885, 9,353,116, WO 2016080750, WO 2016077922, U.S. Pat. Nos. 9,346,813, 9,340,524, 8,566,072, 9,328,112, US 20160113911, WO 2016041618, US 20160060352, WO 2016030439, US 20160046672, WO 2016015605, US 20160022642, WO 2016012982, US 20160016951, US 20140303167, U.S. Pat. Nos. 9,173,938, 9,155,724, US 20150283073, WO 2015154038, WO 2015154064, U.S. Pat. No. 8,518,948, US 20150266878, US 20150259300, US 20150254433, US 20150246946, U.S. Pat. Nos. 9,108,926, 9,096,608, 8,815,879, US 20150174207, U.S. Pat. Nos. 9,062,088, 9,062,039, 8,716,299, US 20080112888, WO 2015066452, U.S. Pat. Nos. 9,044,474, 9,040,529, 9,029,345, 9,023,857, US 20130058987, U.S. Pat. Nos. 9,016,221, 8,999,955, 8,865,176, 8,987,275, 8,841,312, 8,969,375, 8,969,556, US 20150056191, US 20150051227, U.S. Pat. Nos. 8,716,296, 8,946,226, 8,507,511, US 20150010475, US 20140378525, U.S. Pat. Nos. 8,916,557, 8,912,194, US 20140356322, U.S. Pat. No. 8,895,605, WO 2014124258, U.S. Pat. Nos. 8,048,872, 8,546,400, US 20140303386, US 20140303163, WO 2014154723, US 20110287086, U.S. Pat. No. 7,582,642, US 20140287454, WO 2014141289, U.S. Pat. No. 8,822,526, WO 2014128523, U.S. Pat. Nos. 8,809,350, 8,802,686, US 20140221243, US 20140206028, U.S. Pat. No. 8,784,806, US 20140200233, U.S. Pat. No. 8,735,412, US 20140134648, US 20140107481, U.S. Pat. No. 8,691,820, US 20140079665, US 20100215644, U.S. Pat. No. 8,658,662, WO 2013169793, US 20140031325, WO 2014013231, US 20140005070, US 20140004565, WO 2013188355, U.S. Pat. Nos. 8,507,498, 8,252,812, US 20130324530, U.S. Pat. Nos. 8,592,147, 8,592,581, 8,592,583, 8,586,598, WO 2013170147, U.S. Pat. Nos. 8,580,793, 8,569,356, 8,563,741, 8,541,461, US 20130237582, US 20130230856, WO 2013124867, U.S. Pat. No. 8,513,440, US 20130210846, WO 2013106494, US 20130171073, U.S. Pat. No. 8,476,278, WO 2013082660, U.S. Pat. No. 8,450,342, WO 2013071415, U.S. Pat. Nos. 8,435,970, 8,431,583, WO 2013059582, U.S. Pat. No. 8,426,403, WO 2013056132, WO 2013048734, US 20130079345, U.S. Pat. Nos. 8,404,692, 8,404,718, WO 2013036684, U.S. Pat. Nos. 8,389,521, 8,383,813, 8,304,418, 8,367,687, 8,361,467, US 20130023497, U.S. Pat. No. 8,357,673, US 20130017210, WO 2012101065, U.S. Pat. No. 8,344,018, WO 2012168720, U.S. Pat. Nos. 8,258,316, 7,700,609, 8,283,357, 8,277,807, US 20120245158, U.S. Pat. No. 7,531,531, WO 2012123889, US 20120238546, US 20100324327, US 20120220624, WO 2012101064, WO 2012101062, U.S. Pat. Nos. 7,976,517, 8,222,256, 8,216,571, US 20110130380, U.S. Pat. Nos. 8,207,136, 8,207,180, US 20120157433, US 20120156138, US 20120149708, U.S. Pat. No. 8,088,771, US 20120142685, WO 2012069972, WO 2012047017, US 20100129357, WO 2012066065, US 20120121692, U.S. Pat. Nos. 8,134,000, 8,124,764, US 20110151469, U.S. Pat. Nos. 7,605,175, 8,084,027, 8,076,479, 8,067,424, 8,067,461, 7,344,716, US 20110262525, WO 2011127222, US 20110251379, U.S. Pat. Nos. 8,021,831, 8,017,735, 7,998,972, US 20110171203, US 20110160645, US 20110159111, U.S. Pat. Nos. 7,507,734, 7,957,910, US 20110129456, U.S. Pat. No. 7,947,695, US 20110104256, US 20110091524, US 20080176964, U.S. Pat. Nos. 7,279,469, 7,902,361, US 20110053918, US 20090005374, U.S. Pat. Nos. 7,897,619, 7,432,260, US 20110046127, U.S. Pat. No. 7,888,341, US 20030073677, US 20110035814, US 20110014117, WO 2010124009, U.S. Pat. No. 7,863,289, WO 2010136705, US 20100298376, US 20100292320, US 20100286038, U.S. Pat. Nos. 7,816,350, 7,807,705, 7,807,368, US 20100240686, U.S. Pat. Nos. 7,786,306, 7,772,207, 7,517,644, 7,745,450, 7,745,428, 7,078,591, US 20100104534, U.S. Pat. No. 7,700,346, WO 2010034863, U.S. Pat. No. 7,682,785, US 20100063049, US 20100056524, US 20100048597, WO 2010013466, U.S. Pat. No. 7,655,652, US 20100021420, U.S. Pat. Nos. 7,645,775, 7,645,762, US 20100004243, U.S. Pat. No. 7,642,266, US 20090325931, U.S. Pat. No. 7,638,518, US 20090318446, US 20090318430, U.S. Pat. Nos. 7,625,732, 7,157,455, 7,612,079, 7,544,689, 7,067,661, US 20060281736, U.S. Pat. Nos. 7,235,561, 7,226,920, WO 2009115591, US 20090233928, US 20090226431, US 20090221581, US 20090208991, US 20050186261, WO 2009095265, WO 2009047298, U.S. Pat. No. 7,557,110, US 20090170847, WO 2009010298, US 20090142337, US 20090130118, U.S. Pat. No. 7,388,010, WO 2007033208, US 20080188524, US 20090105687, US 20090099160, U.S. Pat. Nos. 7,511,063, 7,511,136, US 20090081645, US 20050209292, US 20090076268, U.S. Pat. No. 7,501,257, WO 2009022104, WO 2009020580, U.S. Pat. No. 7,485,638, US 20090029992, US 20090030005, U.S. Pat. No. 6,610,677, WO 2008132138, U.S. Pat. No. 7,189,716, US 20070238745, US 20080312223, U.S. Pat. Nos. 7,465,728, 6,710,227, US 20080293785, U.S. Pat. Nos. 7,456,191, 6,916,798, WO 2008137139, US 20080280906, U.S. Pat. No. 7,449,544, US 20080275063, U.S. Pat. Nos. 7,446,195, 7,446,105, WO 2008130569, U.S. Pat. No. 7,442,697, WO 2008120098, WO 2008115499, U.S. Pat. Nos. 7,388,015, 7,427,626, WO 2008073304, U.S. Pat. Nos. 7,329,799, 7,407,745, 7,393,953, US 20080153822, US 20080146555, WO 2007044401, U.S. Pat. Nos. 6,821,990, 7,041,824, 7,354,946, 7,348,335, 7,335,674, WO 2008021210, US 20080026992, US 20080027052, U.S. Pat. No. 7,312,225, US 20070287718, WO 2007139732, WO 2007110649, US 20070275382, U.S. Pat. No. 7,300,943, WO 2007123686, U.S. Pat. Nos. 7,288,547, 7,279,473, WO 2007022241, U.S. Pat. No. 7,268,231, WO 2007098090, WO 2007098089, WO 2007097109, WO 2007095389, U.S. Pat. No. 7,258,981, WO 2007054725, US 20070179161, U.S. Pat. No. 7,250,515, WO 2007081060, US 20070167466, US 20070155816, U.S. Pat. Nos. 7,232,826, 7,081,454, 7,208,598, 6,645,990, 6,822,097, US 20070021419, U.S. Pat. No. 7,166,602, US 20070004684, U.S. Pat. Nos. 7,153,964, 6,914,062, US 20060269482, US 20060252748, US 20040147561, US 20060241297, US 20060239973, WO 2006106046, WO 2006105386, U.S. Pat. No. 7,109,220, US 20060194883, US 20060148828, US 20060147922, WO 2006070202, WO 2004066935, U.S. Pat. No. 7,008,953, US 20060142312, U.S. Pat. No. 6,838,464, US 20060135589, US 20050125054, US 20060078535, U.S. Pat. No. 6,635,640, US 20060111378, WO 2006051951, WO 2006024945, U.S. Pat. Nos. 7,026,313, 6,982,260, US 20040077601, US 20050288307, US 20050277656, US 20050276866, US 20050272755, US 20040029151, US 20050267066, U.S. Pat. No. 6,627,633, US 20050261260, US 20050136177, US 20040219214, US 20050222163, U.S. Pat. No. 6,953,783, US 20050222054, U.S. Pat. Nos. 6,949,558, 6,667,311, WO 2005002576, WO 2005083096, WO 2004078925, U.S. Pat. No. 6,939,872, US 20050175592, U.S. Pat. Nos. 6,927,031, 6,899,731, US 20050164976, US 20050153991, U.S. Pat. No. 6,838,558, WO 2005044274, US 20050090529, US 20040082613, US 20050070591, US 20040180844, US 20030157704, U.S. Pat. Nos. 6,863,647, 6,858,709, 6,849,631, US 20050004120, WO 2004113353, US 20040254094, US 20040248905, WO 2004107240, US 20040242869, US 20040225077, U.S. Pat. Nos. 6,812,232, 6,720,427, US 20040185506, US 20040186288, U.S. Pat. No. 6,747,046, US 20040180043, US 20040180848, US 20040176431, US 20040156826, US 20040152651, US 20040138245, U.S. Pat. No. 6,756,385, US 20040110775, US 20040110770, U.S. Pat. Nos. 6,747,128, 6,743,785, WO 2004004730, WO 2004031158, U.S. Pat. No. 6,720,332, WO 2004028571, U.S. Pat. Nos. 6,716,831, 6,713,267, 6,710,052, 6,706,718, US 20040048849, US 20030187007, U.S. Pat. Nos. 6,696,546, 6,683,095, US 20040010027, U.S. Pat. Nos. 6,677,345, 6,630,464, US 20030229105, U.S. Pat. No. 6,569,878, US 20030215861, U.S. Pat. No. 6,649,608, US 20030064426, WO 2003091700, U.S. Pat. Nos. 6,642,231, 6,632,820, 6,620,818, US 20030166016, U.S. Pat. Nos. 6,596,694, 6,586,203, US 20030119816, US 20030113897, US 20030114504, U.S. Pat. Nos. 6,579,903, 6,576,647, 6,573,044, 6,043,030, US 20030049602, US 20030100477, US 20030032177, WO 2003030909, U.S. Pat. No. 6,319,918, WO 2003027299, US 20030060397, U.S. Pat. No. 6,504,034, WO 2002074742, WO 2002053096, US 20030018005, U.S. Pat. No. 6,500,846, WO 2002100401, U.S. Pat. No. 6,486,166, WO 2002072085, U.S. Pat. Nos. 6,462,069, 6,451,618, 6,420,345, WO 2002051849, U.S. Pat. Nos. 6,413,974, 6,414,013, 6,407,103, WO 2001083716, U.S. Pat. Nos. 5,672,508, 6,291,504, WO 2001038532, WO 2001027080, U.S. Pat. Nos. 6,303,618, 6,290,951, WO 2001055148, WO 2001053293, U.S. Pat. Nos. 6,001,868, 6,197,804, WO 1999066055, U.S. Pat. No. 6,013,646, WO 1999043676, U.S. Pat. Nos. 5,767,258, 5,733,920, and any INPADOC family member of each of the above references, each of which is incorporated herein by reference in its entirety. In another embodiment, the CDK inhibitor comprises an inhibitor described in: Alsfouk, A., Journal of Enzyme Inhibition and Medicinal Chemistry, 2021, 36(1):693-706; Goel, B. et al., Curr. Top. Med. Chem., 2020, 20(17):1535-1563; Heptinstall, A. B. et al., Future Med. Chem., 2018, 10(11):1369-1388; Sanchez-Martinez, C. et al., Bioorganic & Medicinal Chemistry Letters, 2019, 29:126637; Di Sante, G. et al., Expert Review of Anticancer Therapy, 2019, 19(7): 569-587; Whittaker, S. R. et al., Pharmacology & Therapeutics, 2017, 173:83-105; Chou, J. et al., Cancer Discovery, 2020, 10:351-370; Galbraith, M. D. et al., Transcription, 2019, 10(2):118-136; Goel, B. et al., Current Topics in Medicinal Chemistry, 2020, 20:1535-1563; Heptinstall, A. B. et al., Future Medicinal Chemistry, 2018, 10(11): 1369-1388; each of which is incorporated herein by reference in its entirety.

In one embodiment, the CDK inhibitor is a CDK9 inhibitor. In one embodiment, the CDK9 inhibitor is Atuveciclib (BAY-1143572) or BAY-1251152 (VIP152). In one embodiment, BAY-1251152 (VIP152) is a selective CDK9 inhibitor while Atuveciclib (BAY-1143572) is a CDK9/PTEFb inhibitor. In one embodiment, the CDK inhibitor is a CDK4/6 inhibitor. In one embodiment, the CDK4/6 inhibitor is Palbociclib. In one embodiment, the CDK inhibitor is a CDK7 inhibitor. In one embodiment, the CDK7 inhibitor is THZ1.

Exemplary CDK inhibitors include, but are not limited to: Compound 21 (PMID 27326333) CYC065; YKL-1-116; i-CDK9; JH-VII-49; JH-XI-10-02; SEL120-34A; MM-D37K; PF-06873600; BEY-1007; BEY-1107; birociclib (XZP-3297); FCN-437; TP-1287; BEBT-209; TQB-3616; AMG-925 (FLX-925); CS3002; HS-10342; terameprocol (EM-1421); NU-6102; CGP-60474; BMS-265246; NU-6027; Purvalanol A; Purvalanol B; RGB-286147; Indirubin; 7-Hydroxystaurosporine; BS-194; PHA-690509; Inhibitor IV, FCN437c;

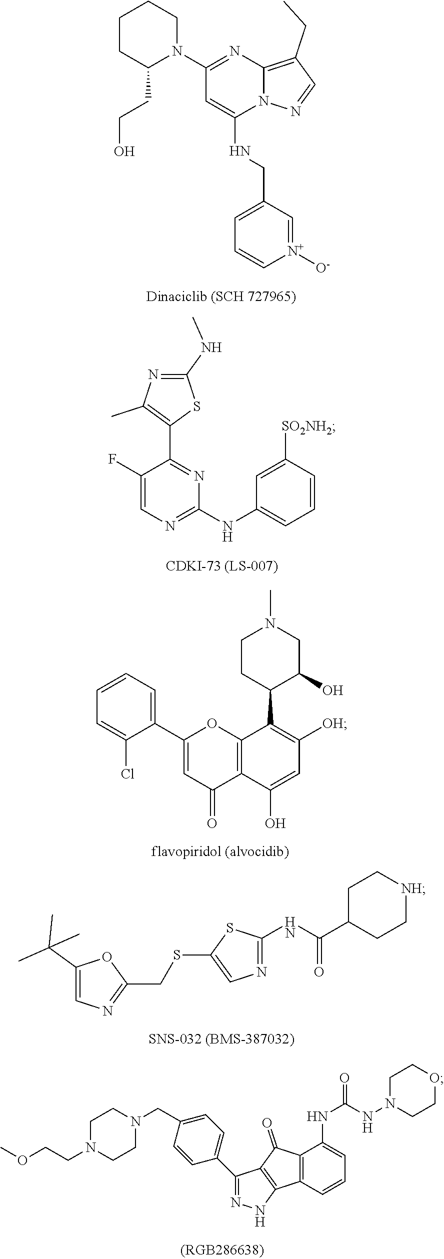 [SMILES: CCc1cnn2c(NCc3ccc[n+]([O-])c3)cc(N3CCCC[C@H]3CCO)nc12 ; CNc1nc(C)c(-c2nc(Nc3cccc(S(N)(=O)=O)c3)ncc2F)s1 ; CN1CC[C@H](c2c(O)cc(O)c3c(=O)cc(-c4ccccc4Cl)oc23)[C@H](O)C1 ; CC(C)(C)c1cnc(CSc2cnc(NC(=O)C3CCNCC3)s2)o1 ; COCCN1CCN(Cc2ccc(-c3n[nH]c4c3C(=O)c3c(NC(=O)NN5CCOCC5)cccc3-4)cc2)CC1]

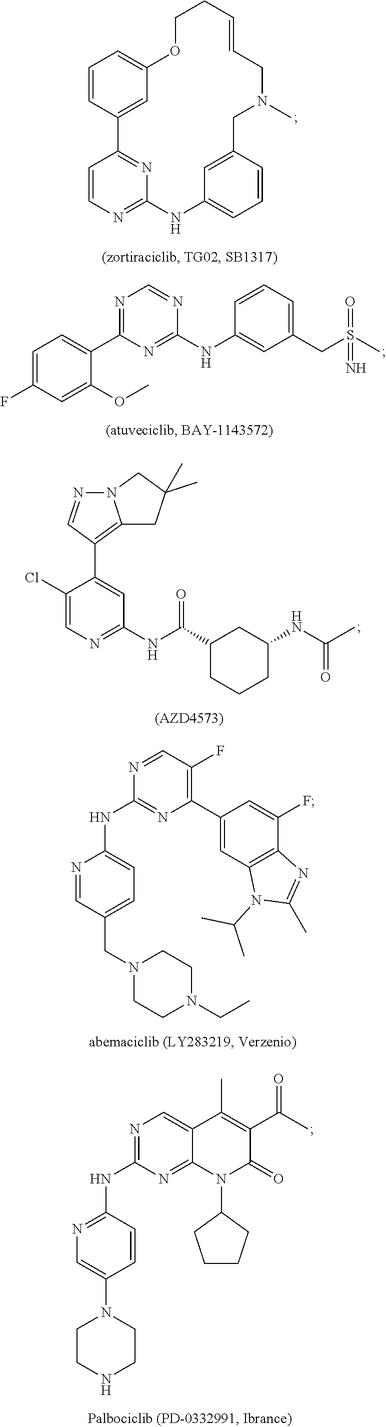

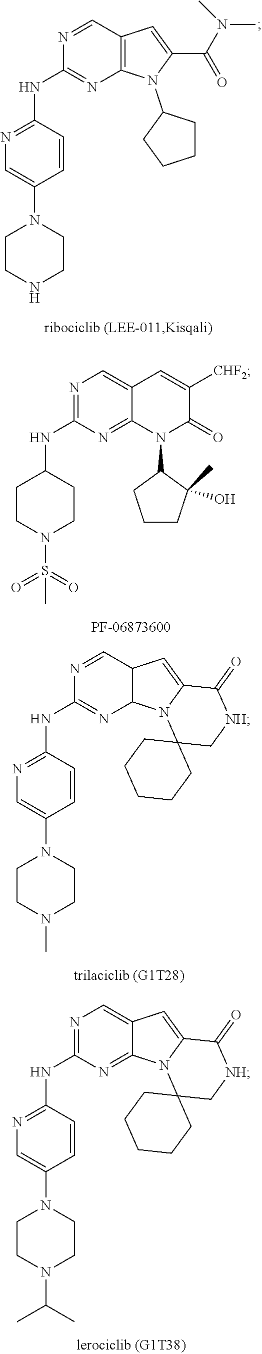

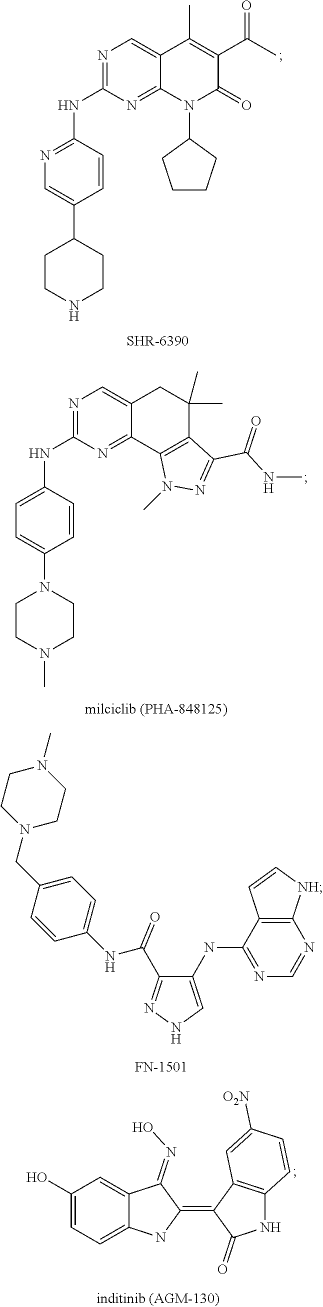

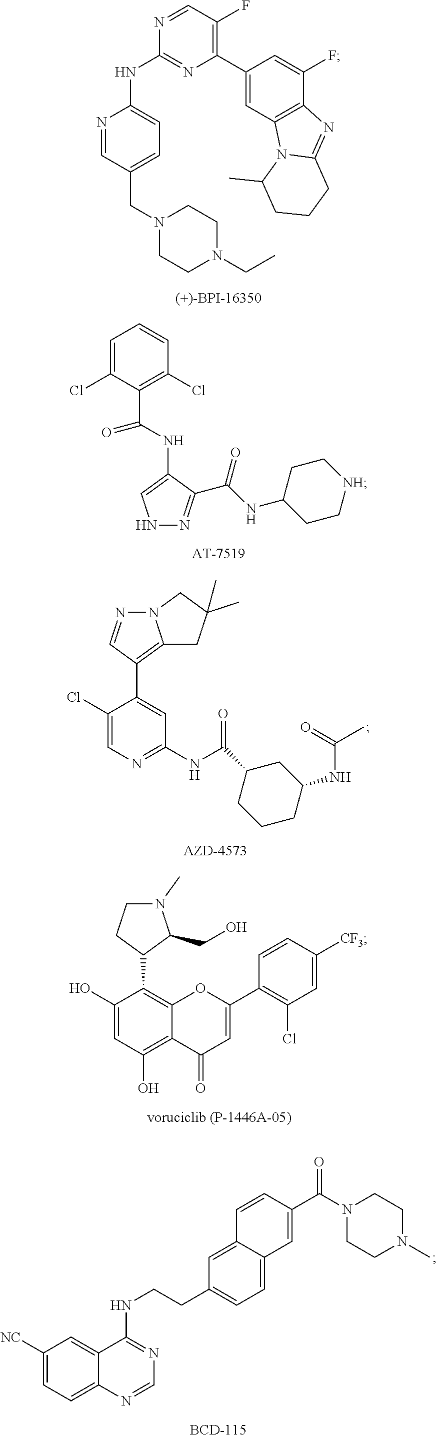

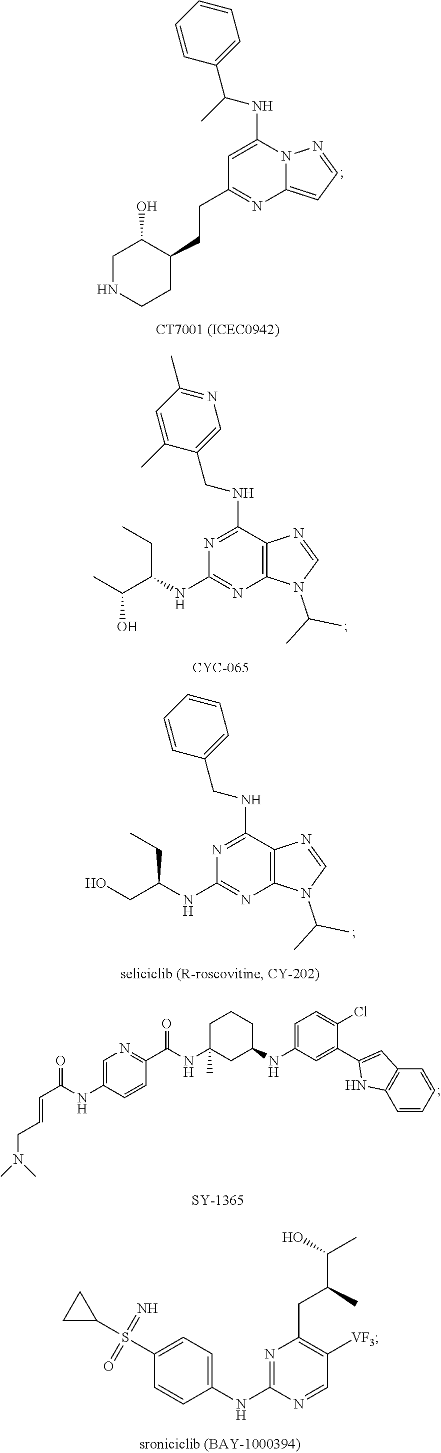

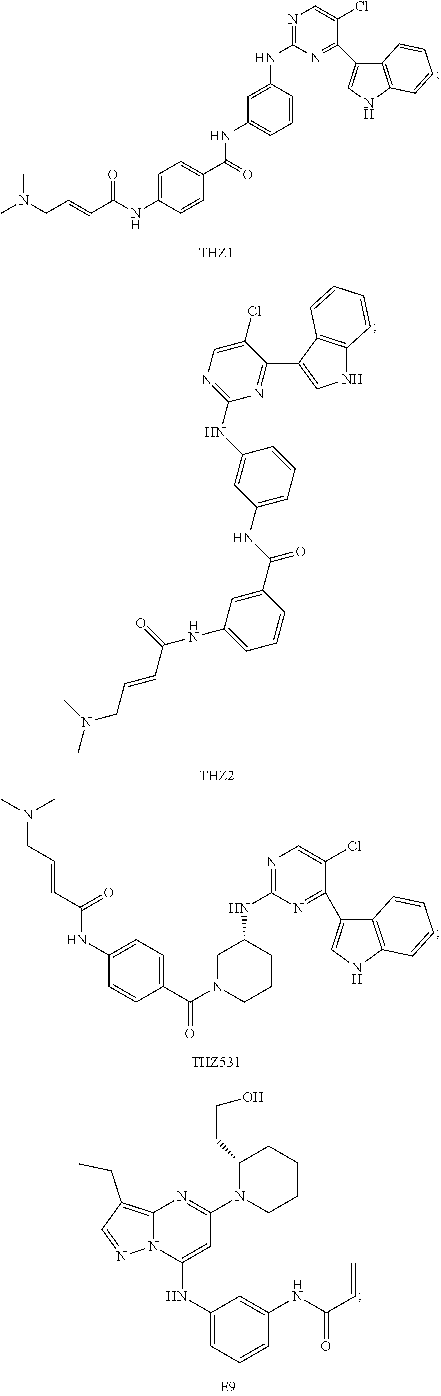 [SMILES: CN(C)C/C=C/C(=O)Nc1ccc(C(=O)Nc2cccc(Nc3ncc(Cl)c(-c4c[nH]c5ccccc45)n3)c2)cc1 ; CN(C)C/C=C/C(=O)Nc1cccc(C(=O)Nc2cccc(Nc3ncc(Cl)c(-c4c[nH]c5ccccc45)n3)c2)c1 ; CN(C)C/C=C/C(=O)Nc1ccc(C(=O)N2CCC[C@@H](Nc3ncc(Cl)c(-c4c[nH]c5ccccc45)n3)C2)cc1 ; C=CC(=O)Nc1cccc(Nc2cc(N3CCCC[C@H]3CCO)nc3c(CC)cnn23)c1]

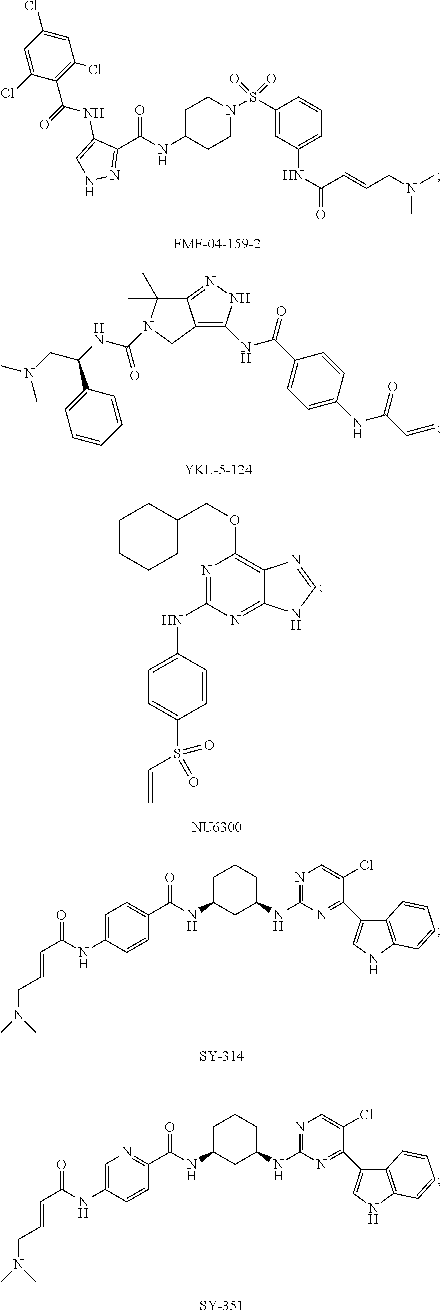

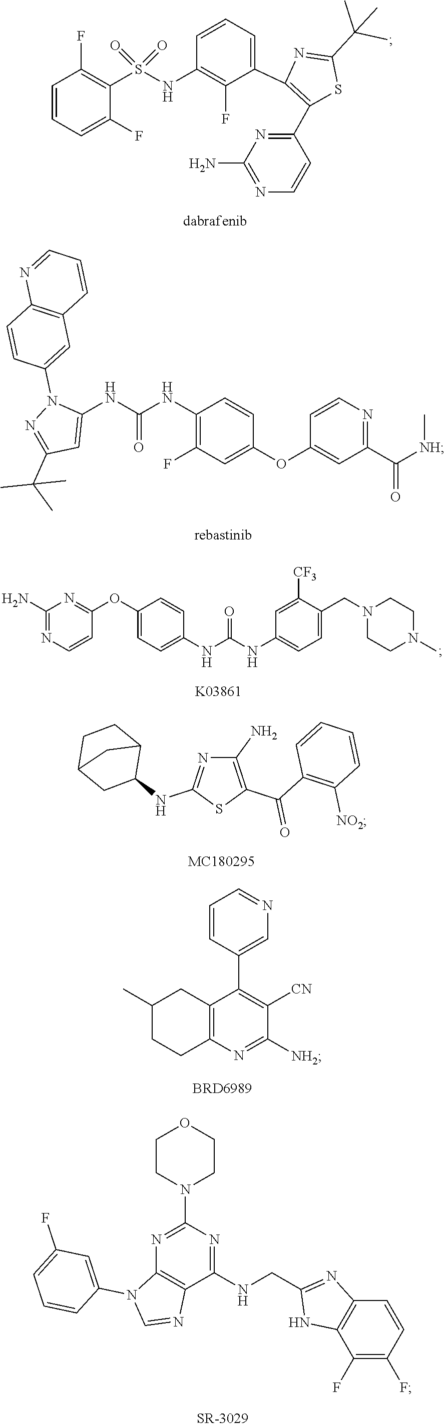

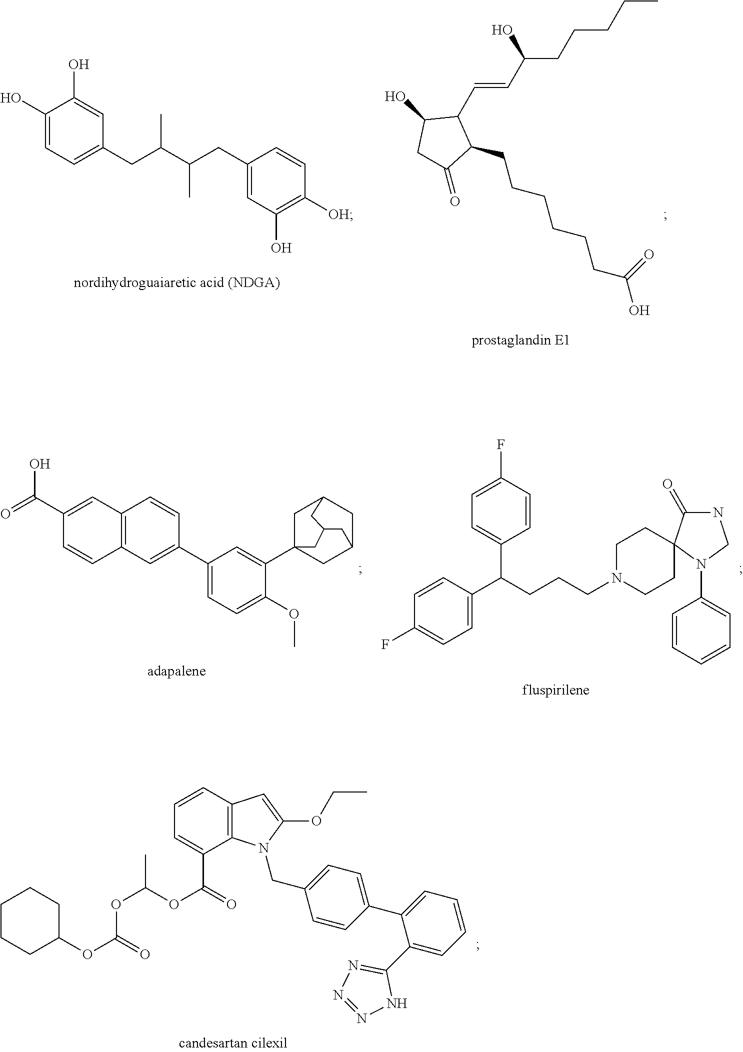 [SMILES: CC(Cc1ccc(O)c(O)c1)C(C)Cc1ccc(O)c(O)c1 ; CCCCC[C@H](O)/C=C/C1[C@H](O)CC(=O)[C@@H]1CCCCCCC(=O)O ; COc1ccc(-c2ccc3cc(C(=O)O)ccc3c2)cc1C12CC3CC(CC(C3)C1)C2 ; O=C1NCN(c2ccccc2)C12CCN(CCCC(c1ccc(F)cc1)c1ccc(F)cc1)CC2 ; CCOc1cc2cccc(C(=O)OC(C)OC(=O)OC3CCCCC3)c2n1Cc1ccc(-c2ccccc2-c2nnn[nH]2)cc1]

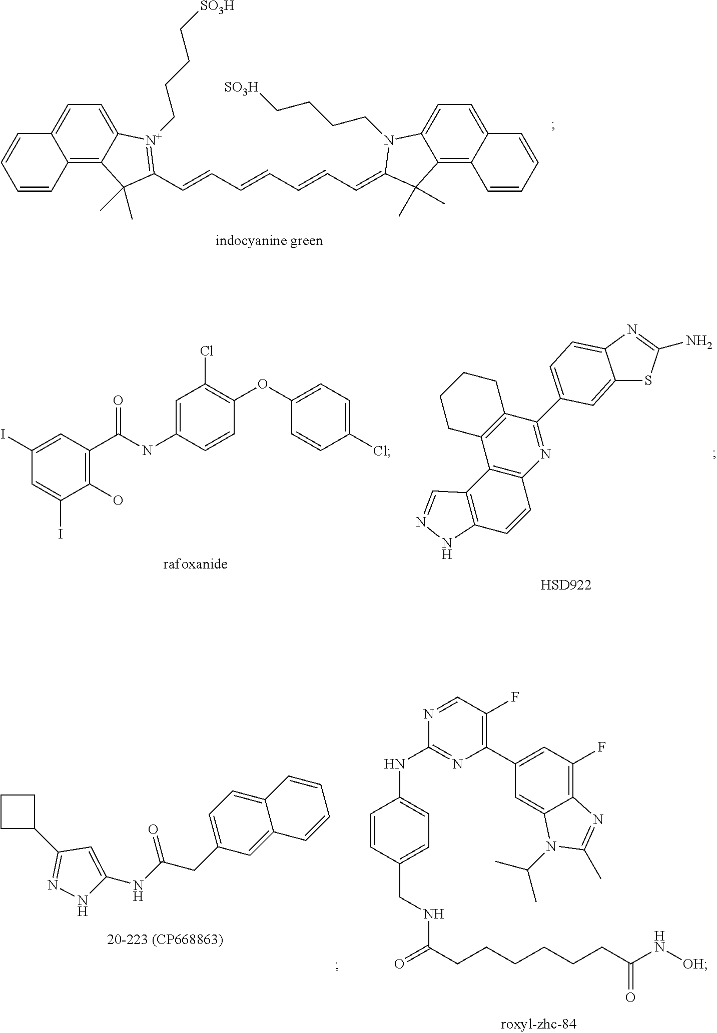

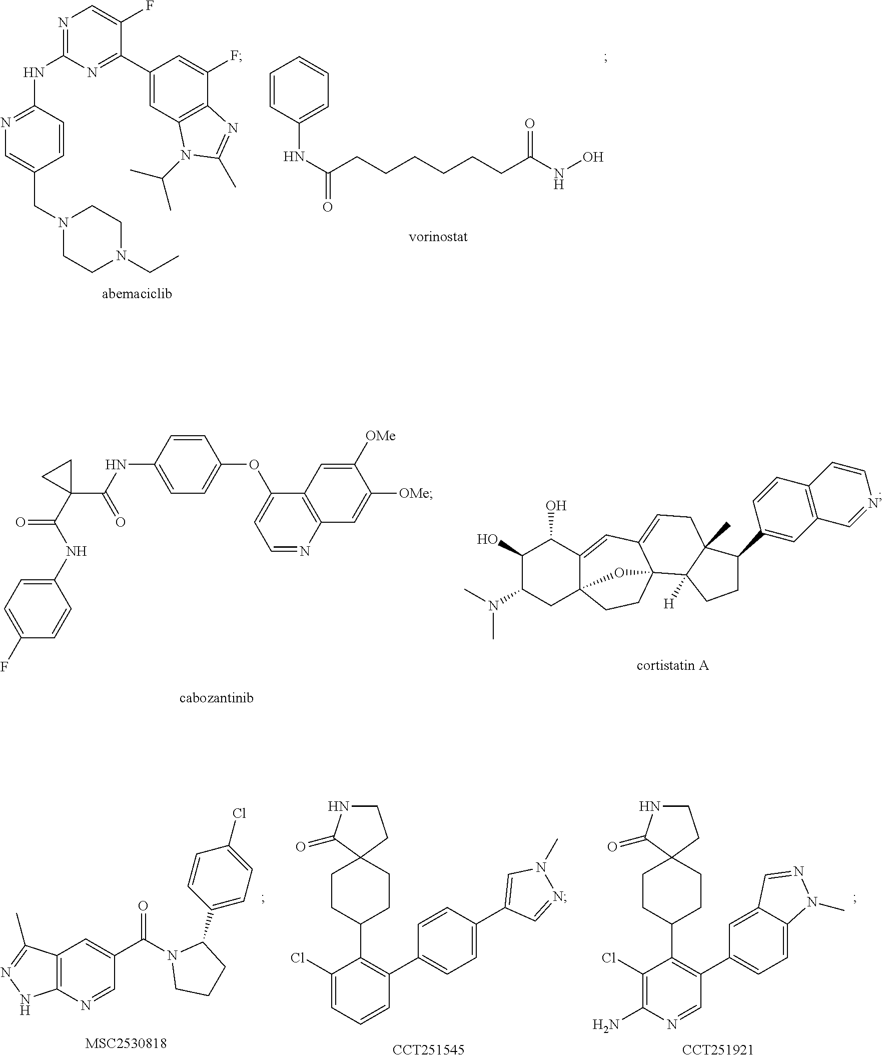

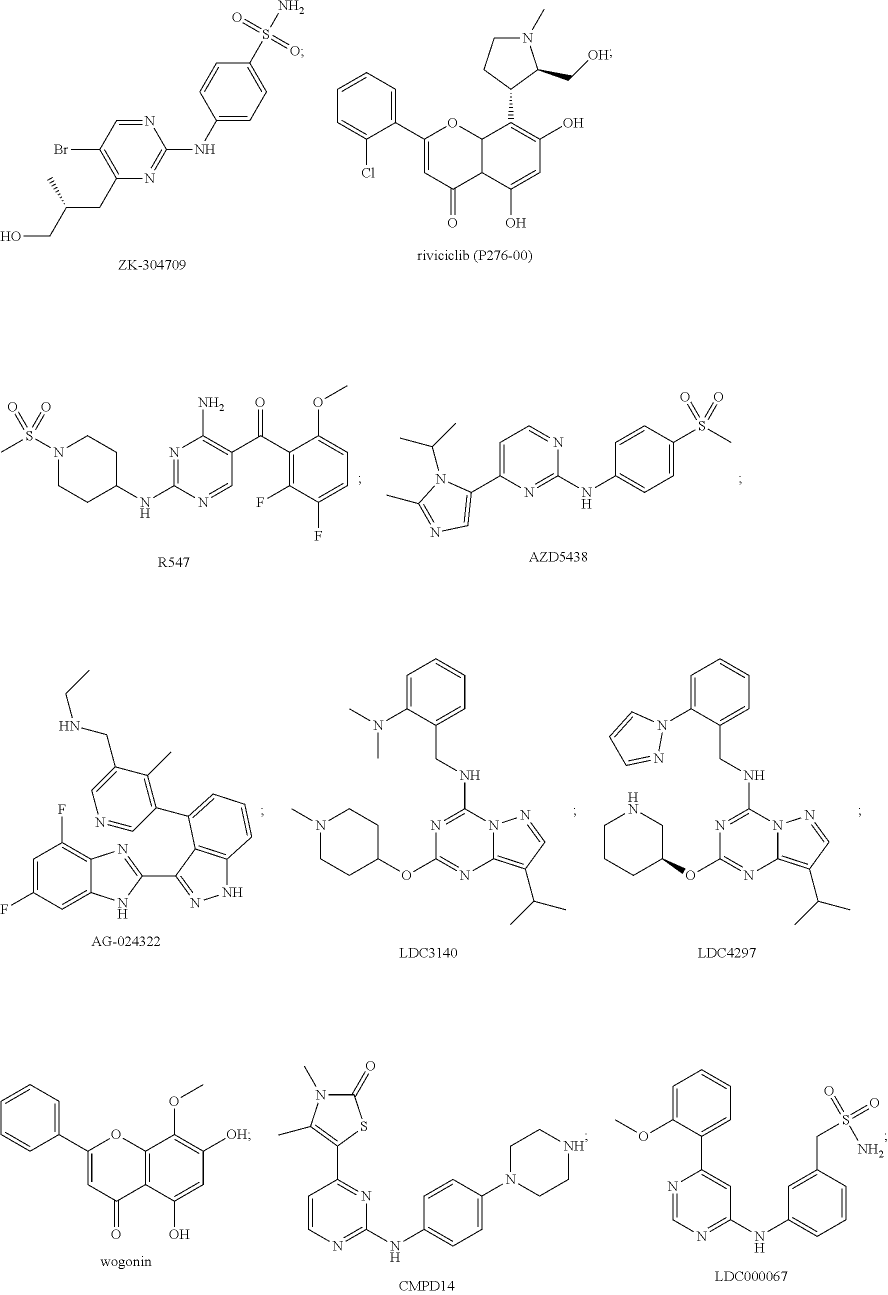

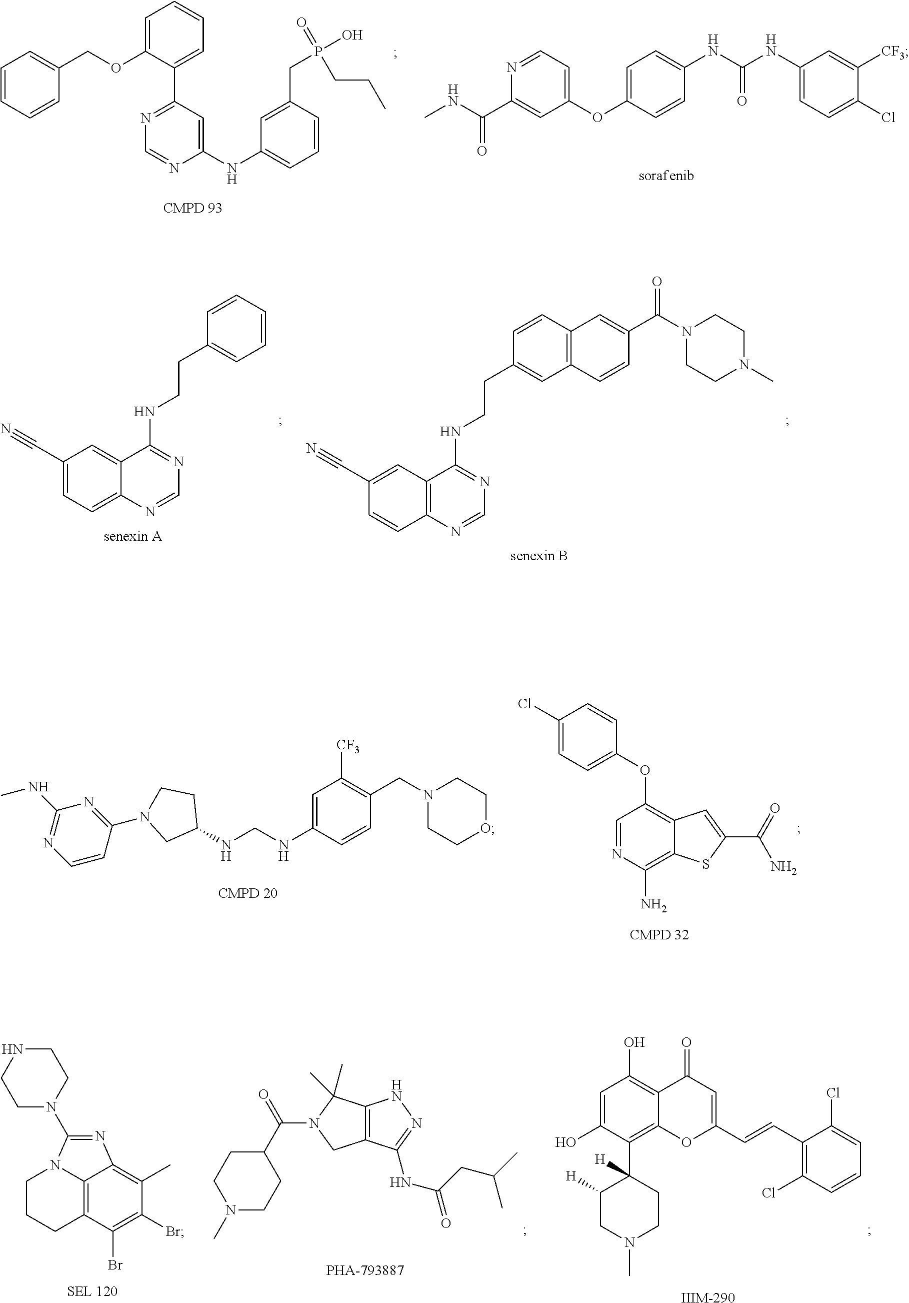

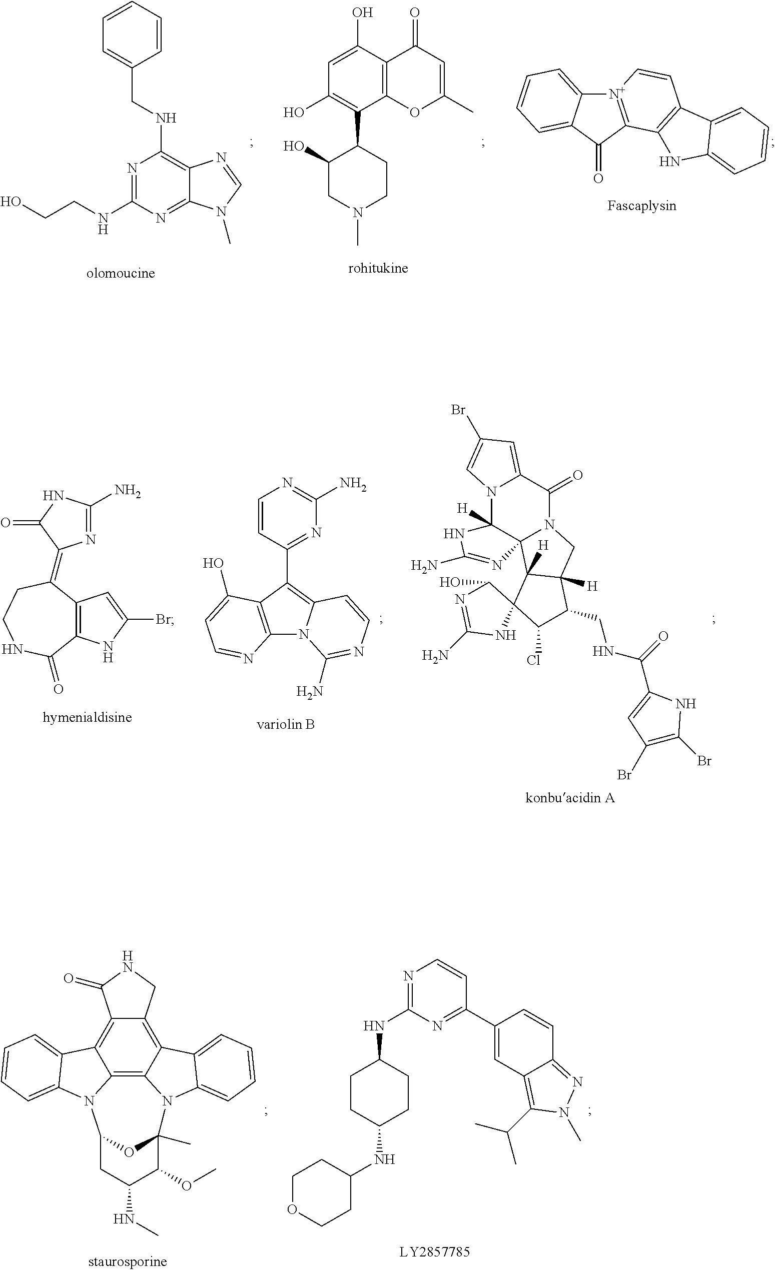

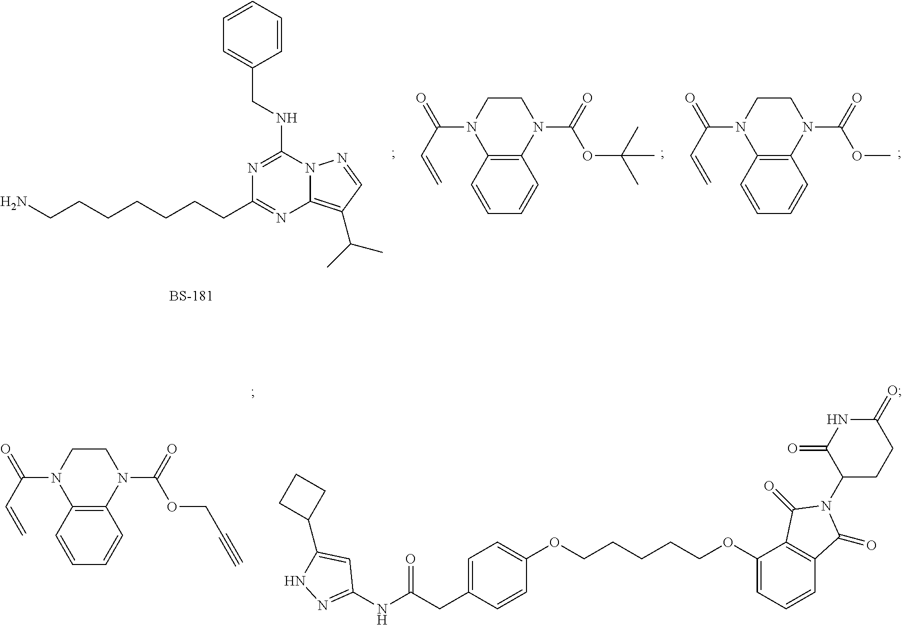

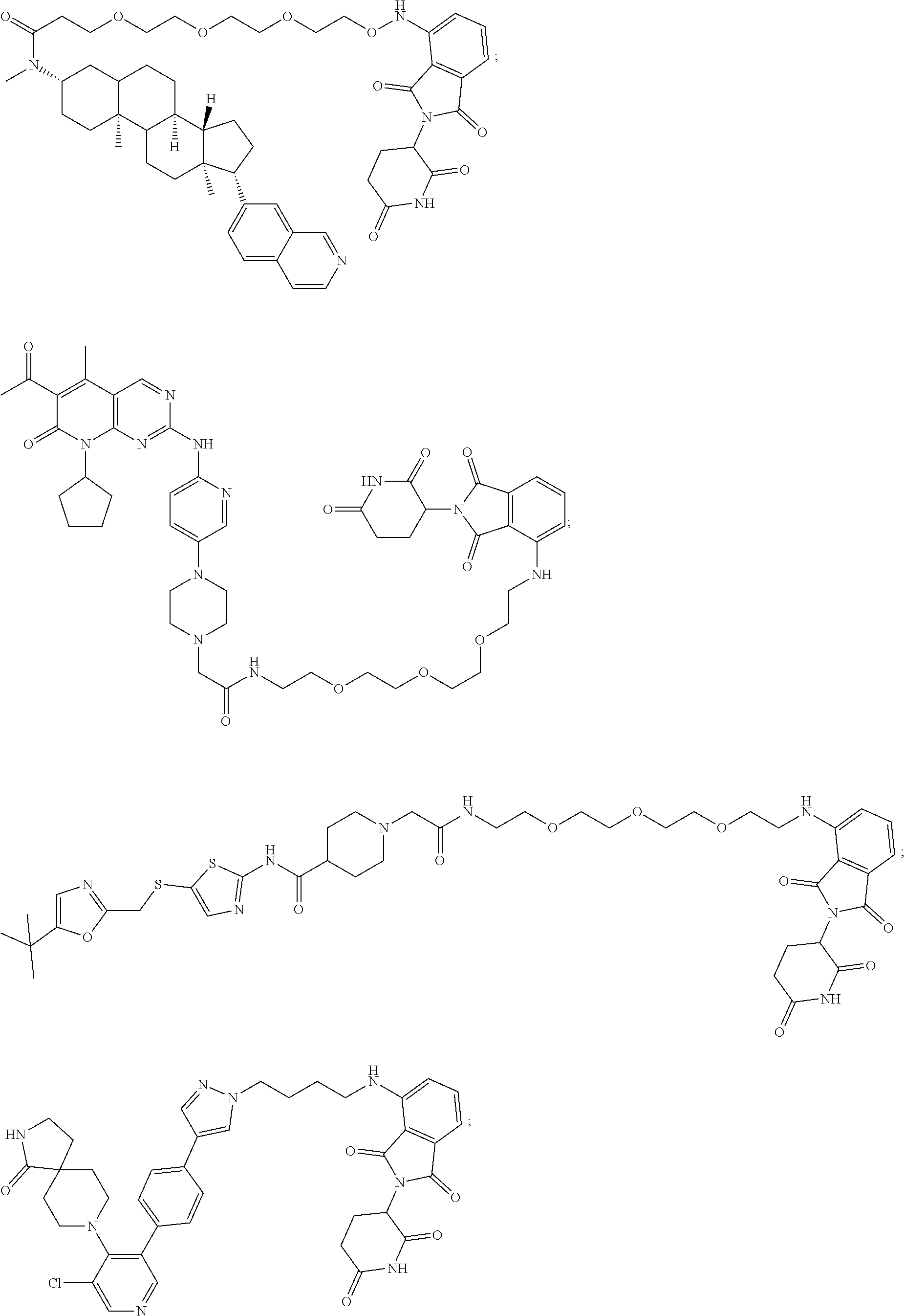

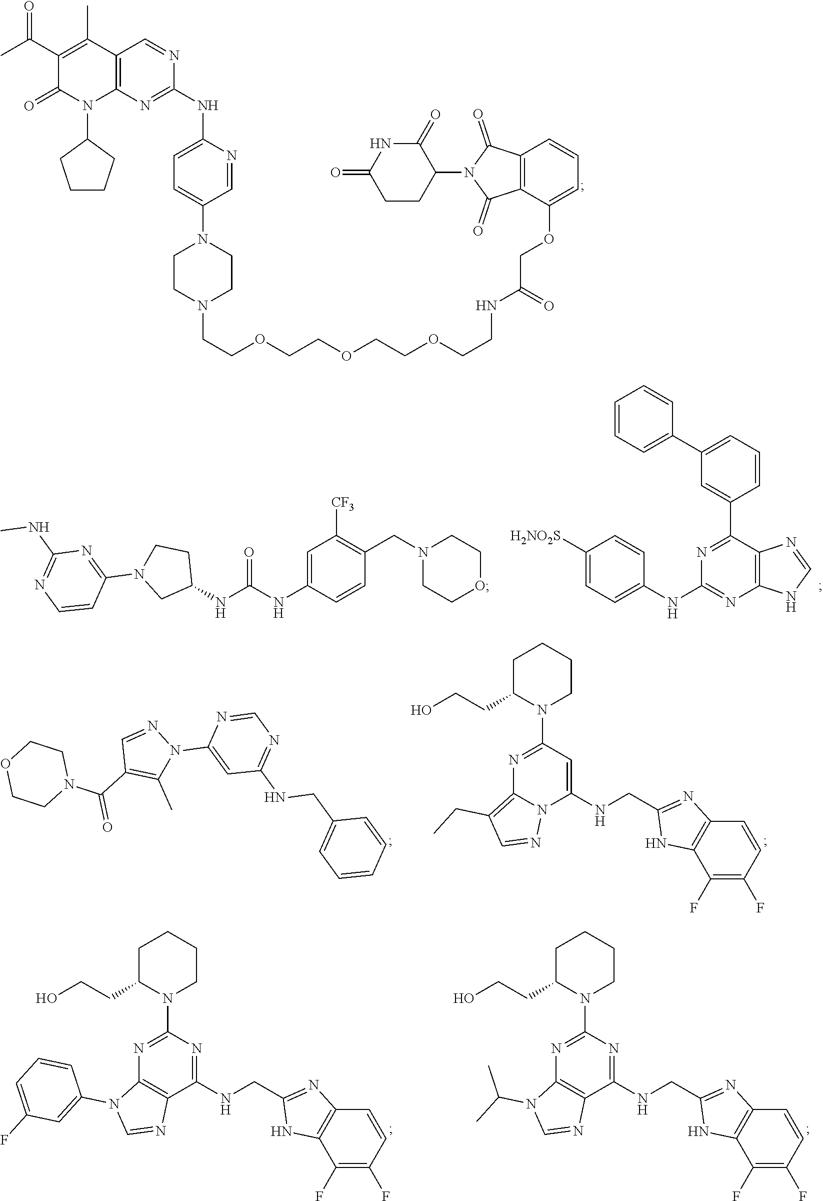

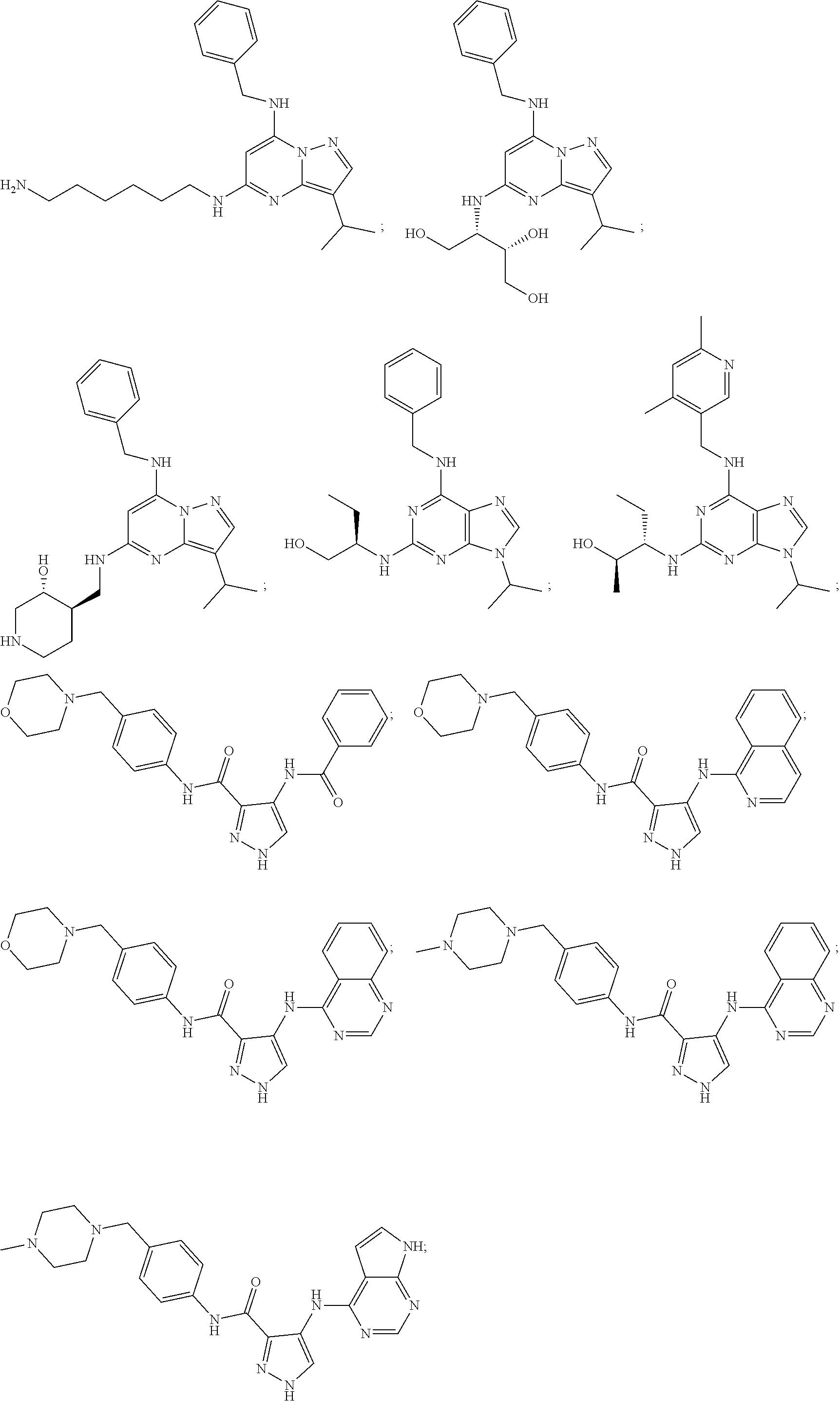

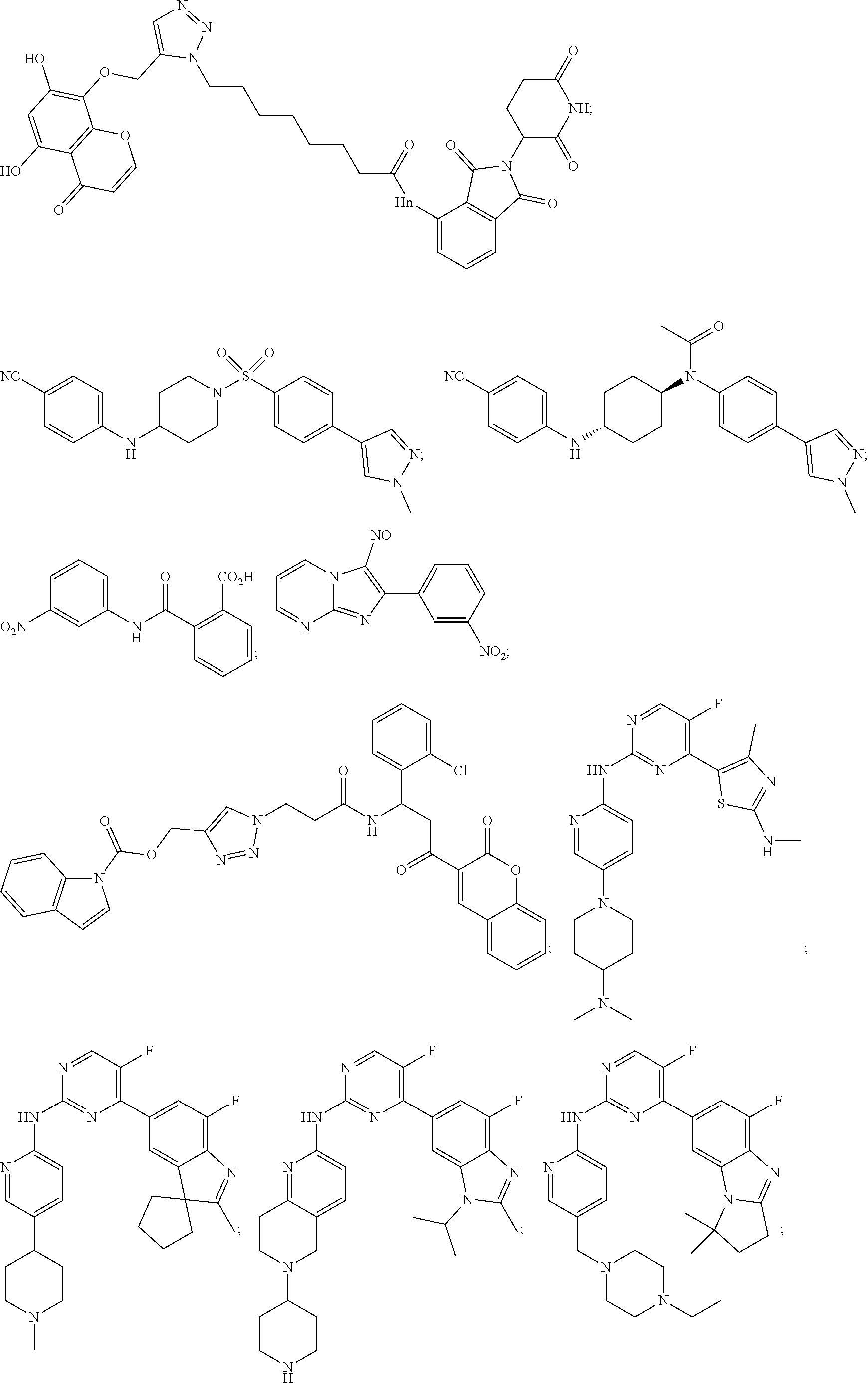

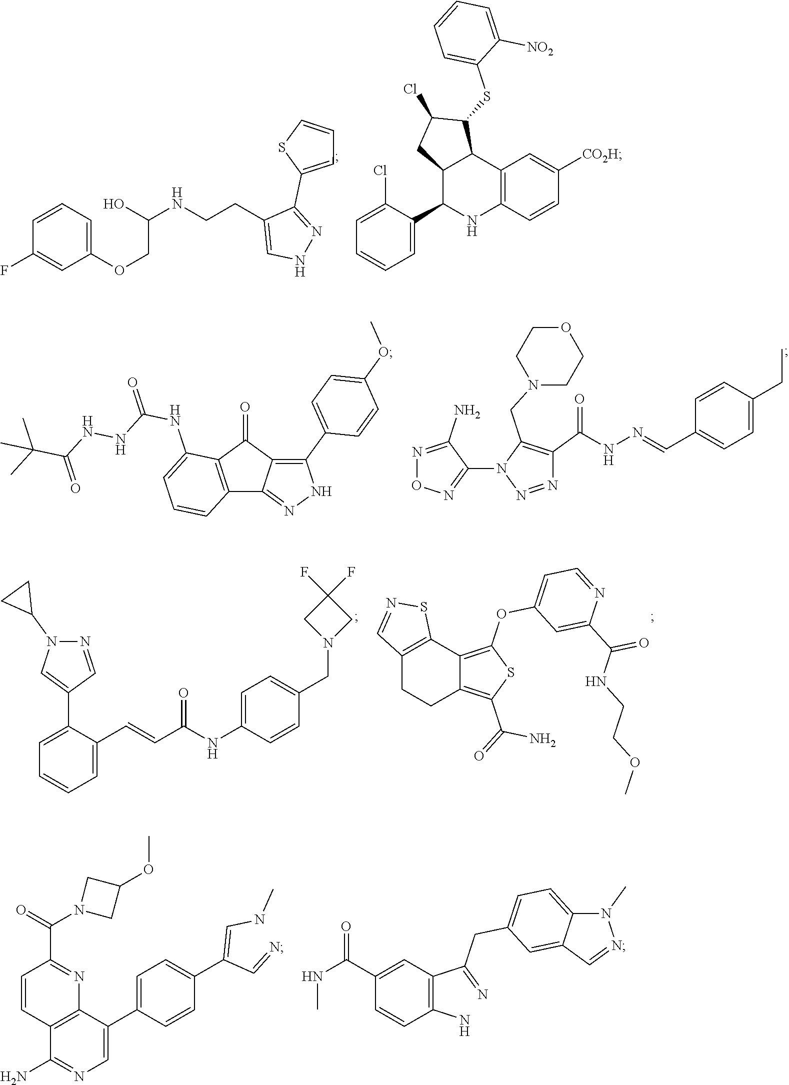

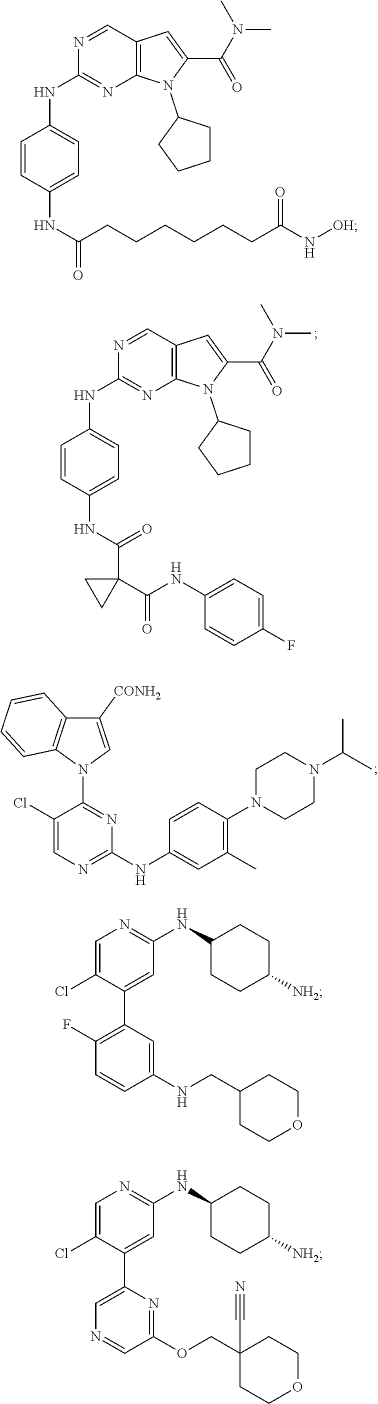

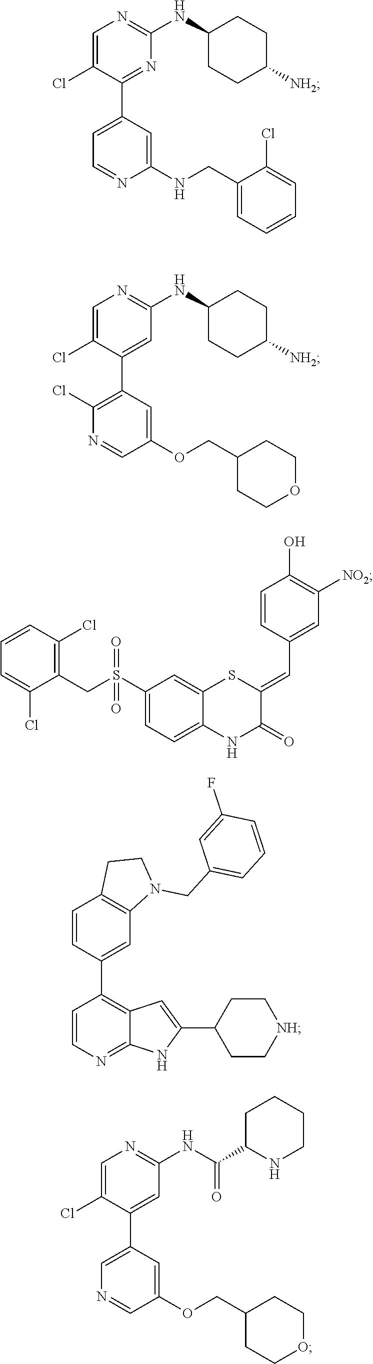

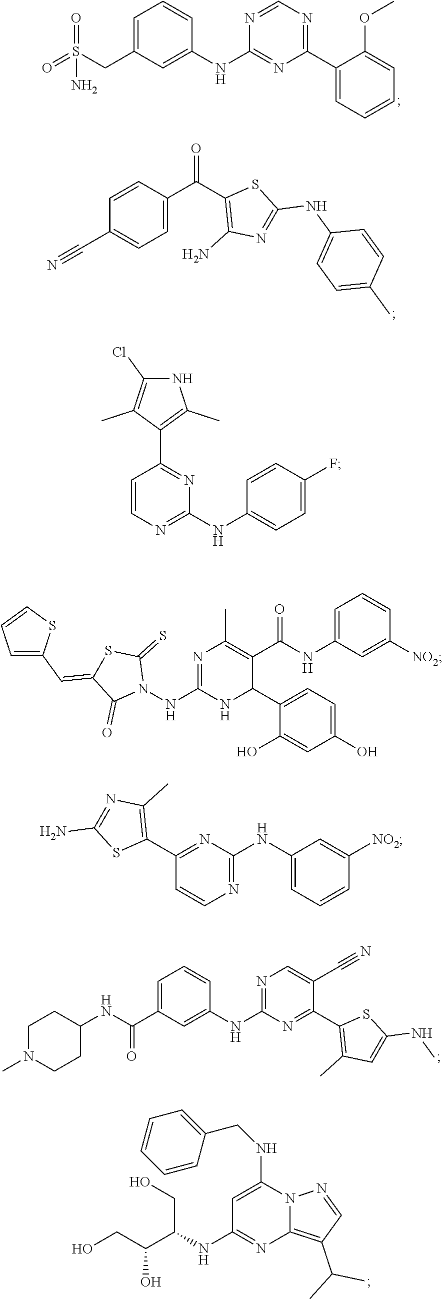

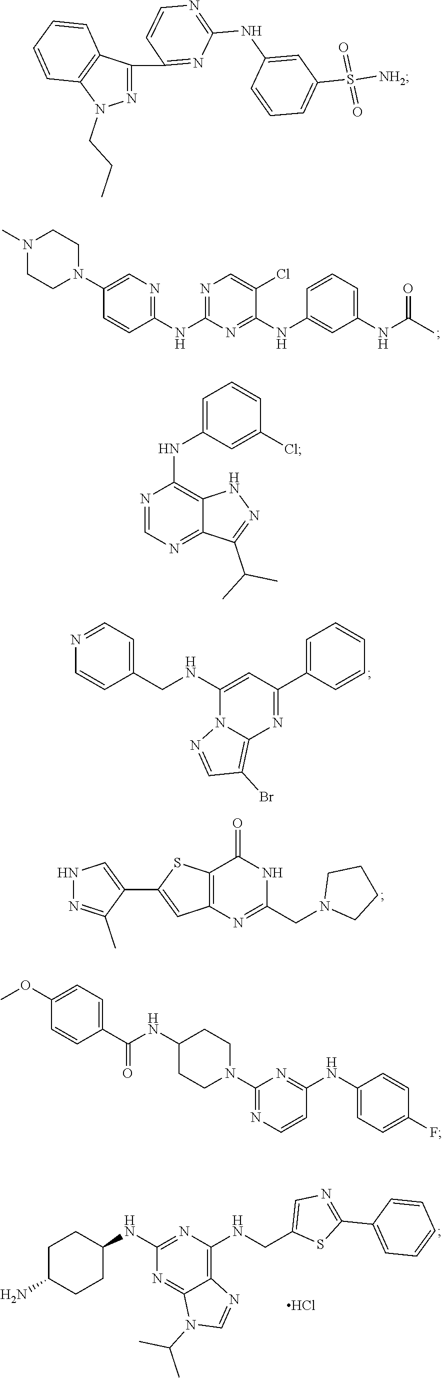

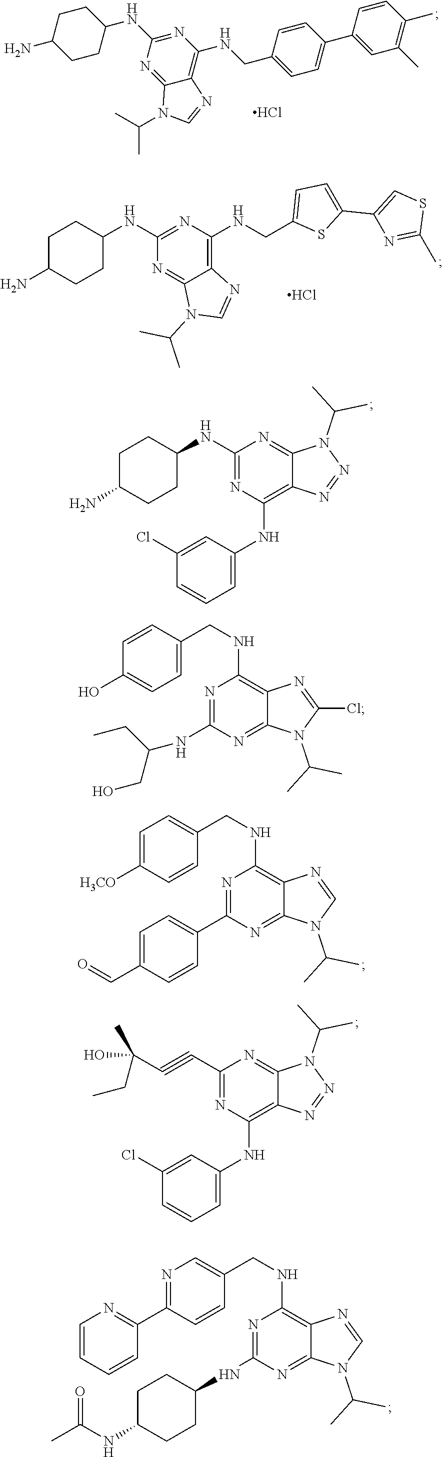

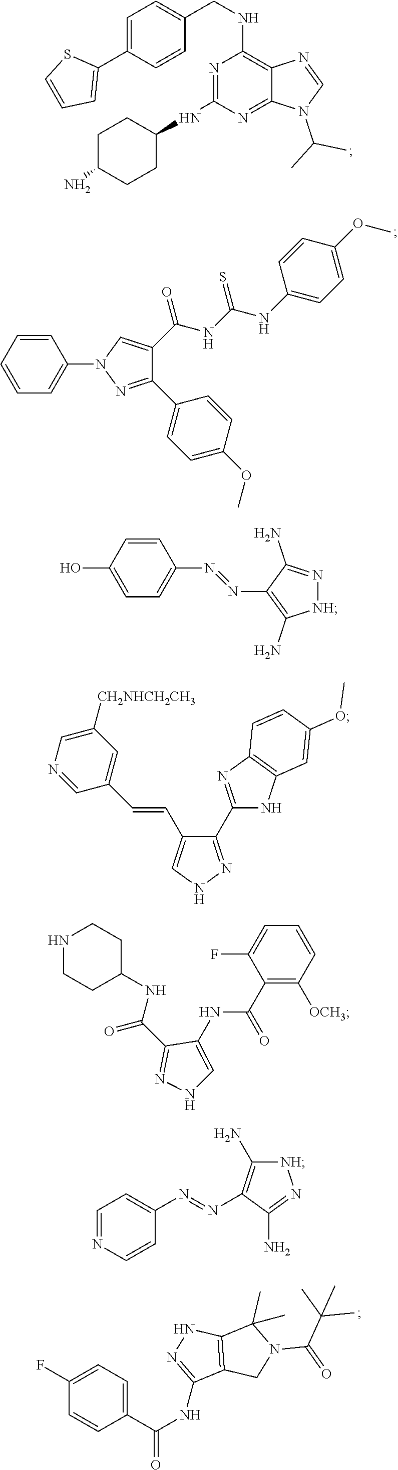

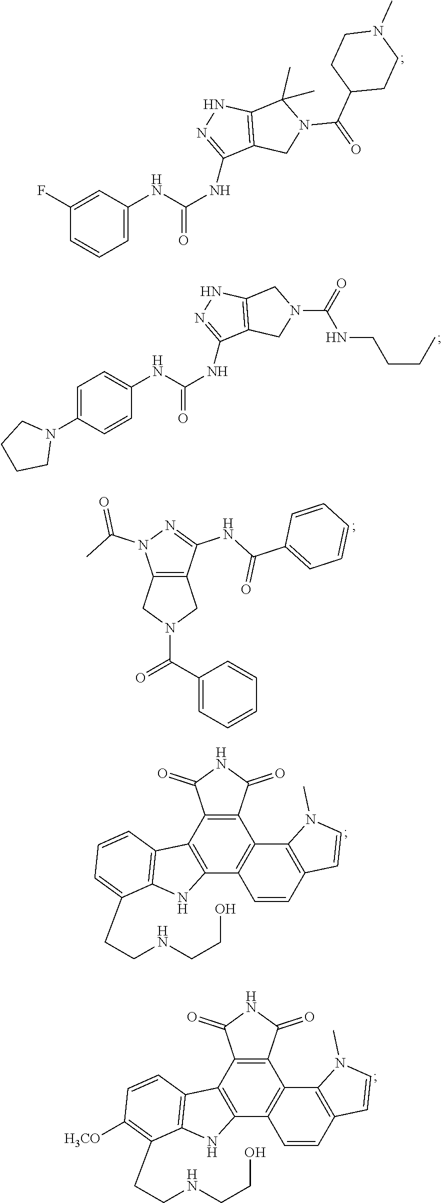

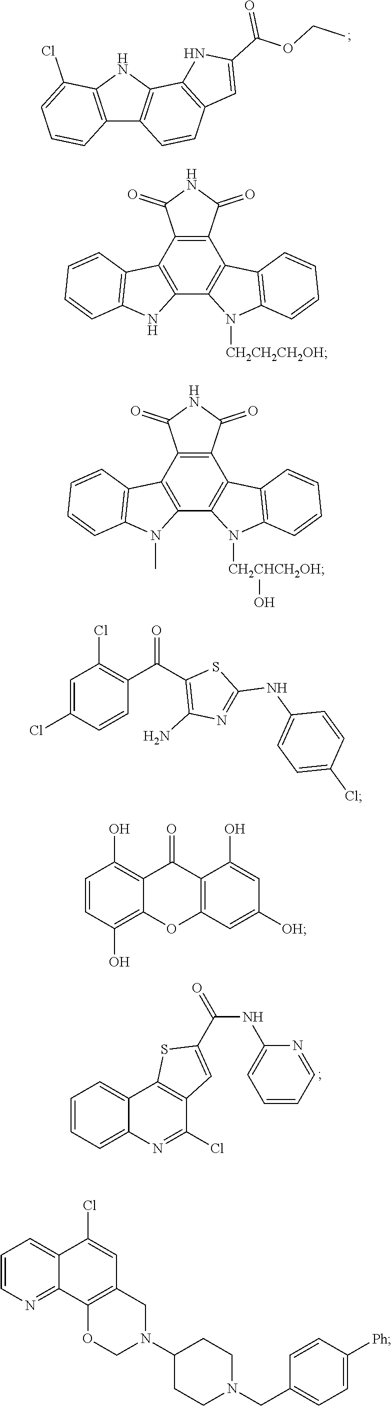

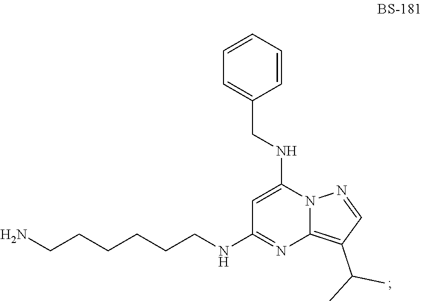 [SMILES: CC(C)c1cnn2c(NCc3ccccc3)cc(NCCCCCCN)nc12]

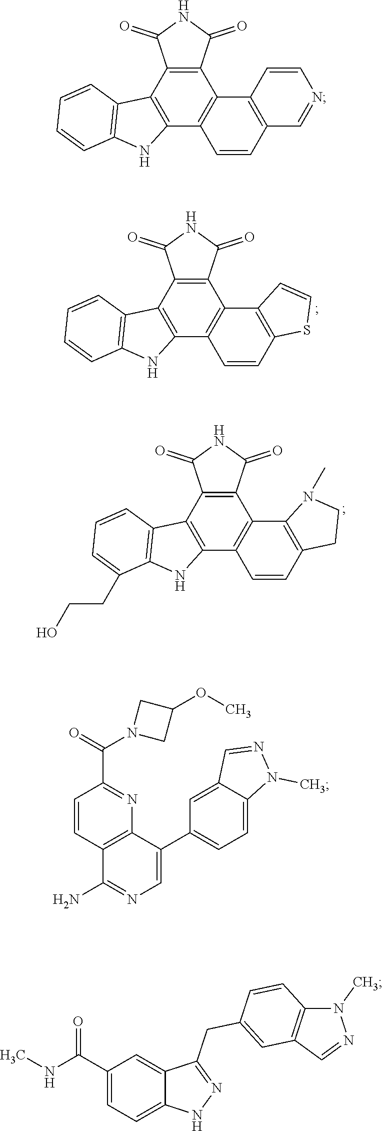 [SMILES: O=C1NC(=O)c2c1c1c3ccncc3ccc1c1[nH]c3ccccc3c21 ; O=C1NC(=O)c2c1c1c3ccsc3ccc1c1[nH]c3ccccc3c21 ; CN1CCc2ccc3c(c4c(c5c6cccc(CCO)c6[nH]c35)C(=O)NC4=O)c21 ; COC1CN(C(=O)c2ccc3c(N)ncc(-c4ccc5c(cnn5C)c4)c3n2)C1 ; CNC(=O)c1ccc2[nH]nc(Cc3ccc4c(cnn4C)c3)c2c1]

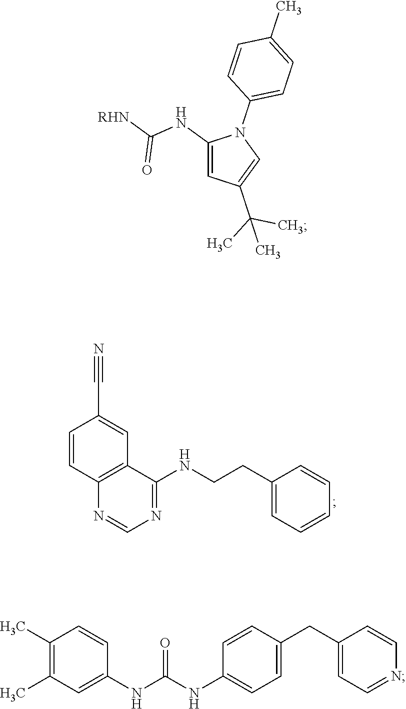

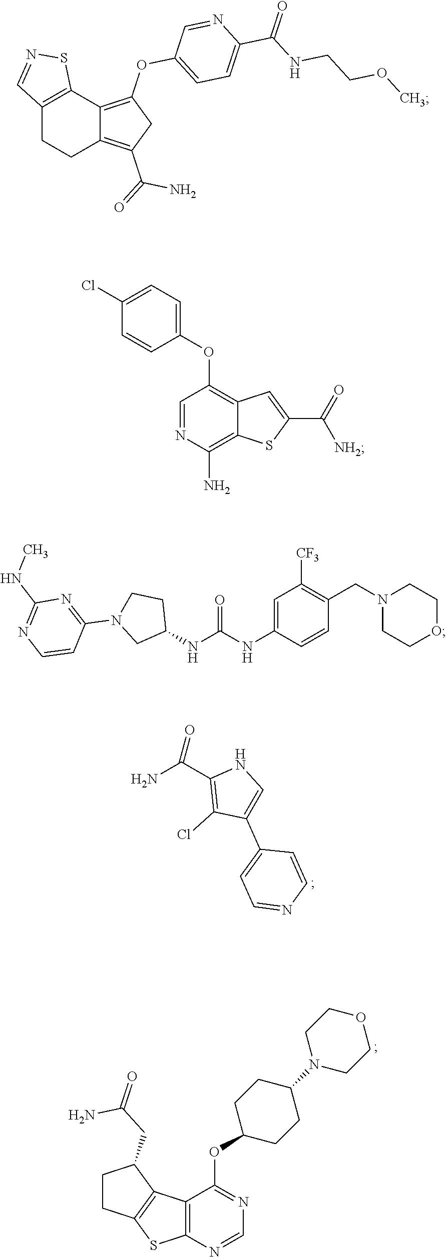 [SMILES: COCCNC(=O)c1ccc(OC2=C3C(=C(C(N)=O)C2)CCc2cnsc23)cn1 ; NC(=O)c1cc2c(Oc3ccc(Cl)cc3)cnc(N)c2s1 ; CNc1nccc(N2CC[C@H](NC(=O)Nc3ccc(CN4CCOCC4)c(C(F)(F)F)c3)C2)n1 ; NC(=O)c1[nH]cc(-c2ccncc2)c1Cl ; NC(=O)C[C@H]1CCc2sc3ncnc(O[C@H]4CC[C@H](N5CCOCC5)CC4)c3c21]

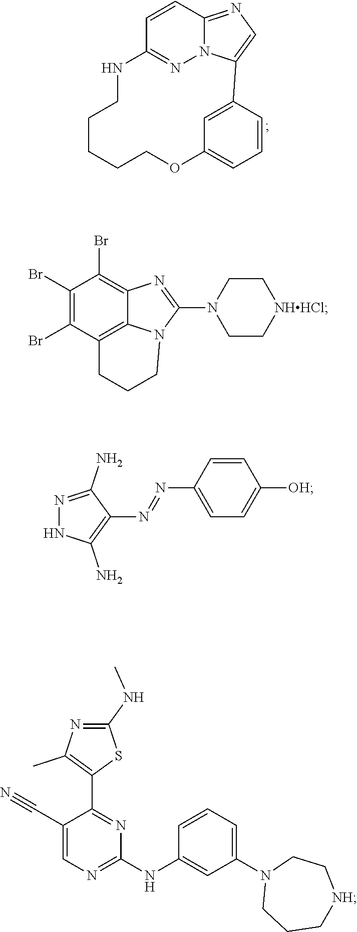

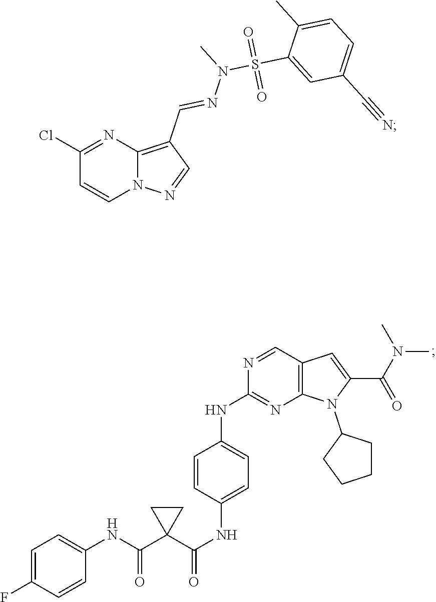 [SMILES: Cc1ccc(C#N)cc1S(=O)(=O)N(C)/N=C/c1cnn2ccc(Cl)nc12 ; CN(C)C(=O)c1cc2cnc(Nc3ccc(NC(=O)C4(C(=O)Nc5ccc(F)cc5)CC4)cc3)nc2n1C1CCCC1]

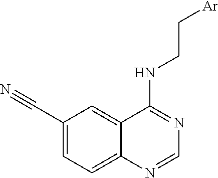

N wherein Ar is

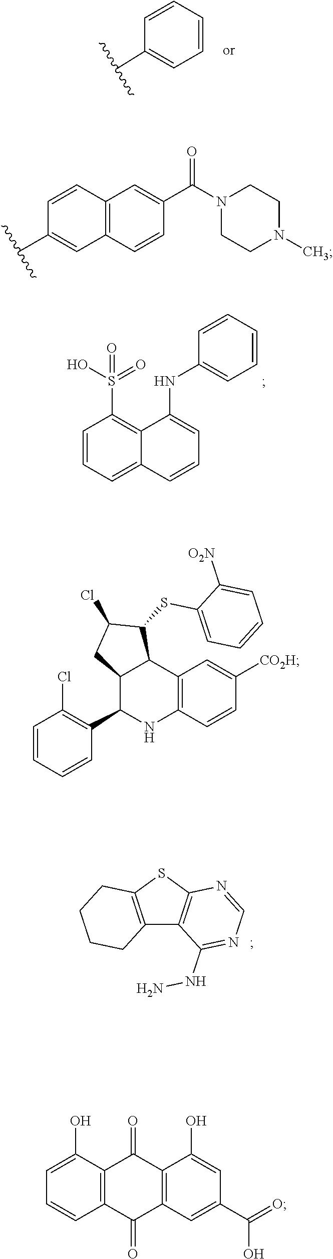

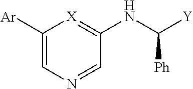

wherein X is N, Y is —C(═O)H, and Ar is

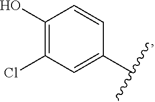 [SMILES: *c1ccc(O)c(Cl)c1]

### X is N, Y is —CH₂OH, and Ar is

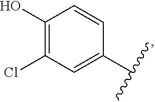 [SMILES: *c1ccc(O)c(Cl)c1]

### or X is CH, Y is —CH₂OH, and Ar is;

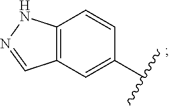

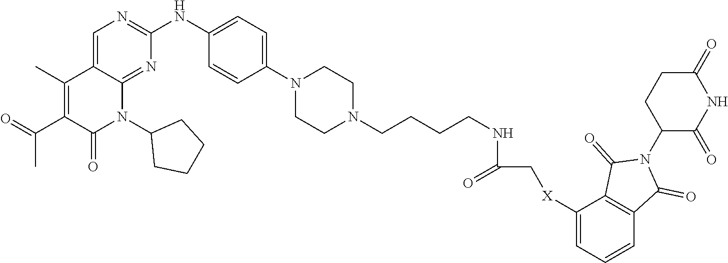

wherein X is NH or O;

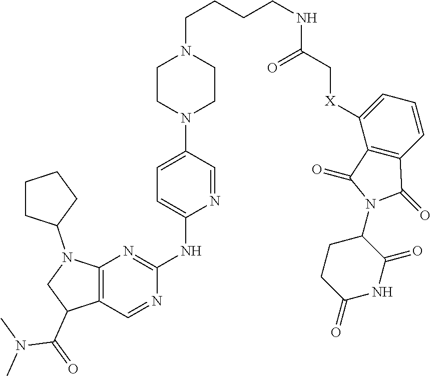

wherein X is NH or O;

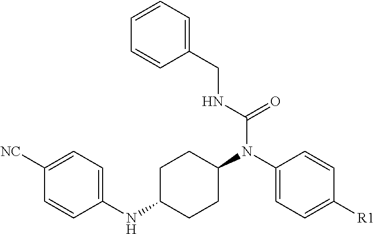

wherein R¹ is or

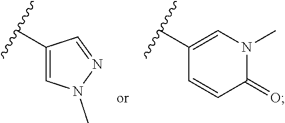

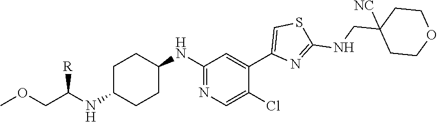

wherein R is H or —CH₃;

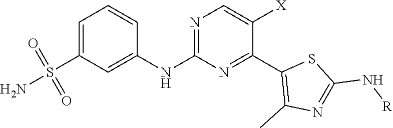

wherein R is —CH₃ and X is F, R is H and X is F, or R is —CH₃ and X is Cl;

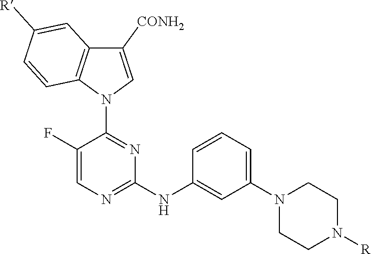

wherein R is tetrahydro-pyran-4-yl and R′ is H, R is —CH₂CH₃ and R′ is —OCH₃. R is isopropyl and R′ is H, or R is —CH₂CH₃ and R′ is F;

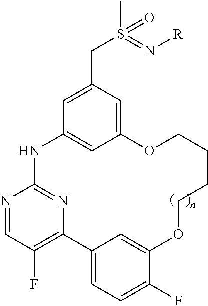

wherein R is t-butyl carboxyl and n is 1 or R is H and n is 2;

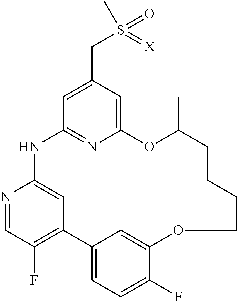

wherein X is NH or 0;

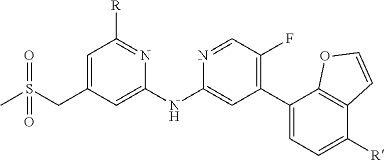

wherein R is H and R′ is F, R is F and R′ is F, or R is H and R′ is H;

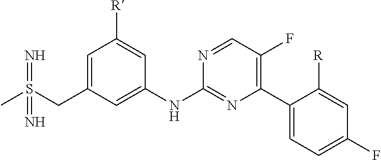

wherein R is —OCH₃ and R′ is F, R is F and R′ is SF₅, or R is —OCH₃ and R′ is —SF₅;

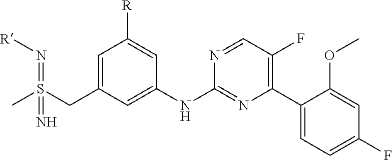

wherein R is F and R′ is —CH₃ or R is —SF₅ and R′ is H;

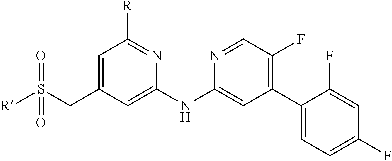

wherein R is —CF₃ and R′ is —CH₃ or R is H and R′ is cyclopropyl;

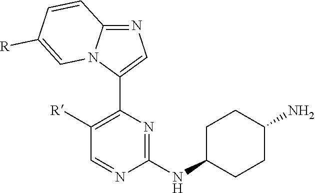

wherein R is 3-fluoroailin-1yl and R′ is F or R is phenyl and R′ is —CH₃;

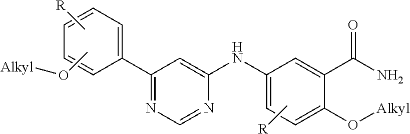

wherein R is H or F and Alkyl is —CH₃ or —CH₂CH₃;

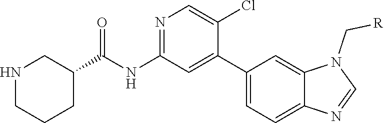

wherein R is 3-fluorophenyl or morpholin-4yl;

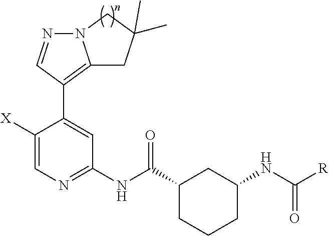

wherein R is cyclopropan-1-ol-1-yl, X is Cl, and n is 1 or R is tetrahydrofuran-3yl, X is Cl and n is 1, or R is —CH₃, X is F and n is 2, or R is cyclopropane-1-1-yl, X is F and n is 1, or oxatan-3-yl, X is —CH₃, and n is 1;

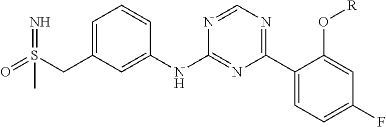

wherein R is 1,2-oxazol-3yl or 3,4-difluorobenzen-1yl;

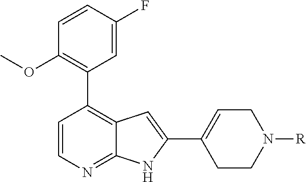

wherein R is H, C(═O)NHCH₃, —SO₂NH₂, SO₂CH₃, or 2,3-dihydroxpropan-1yl;

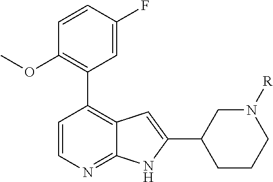

wherein R is H, CH₃, 2-aminoethyan-1yl, 3-aminopropan-1yl, or 2,3-dihydroxpropan-1yl;

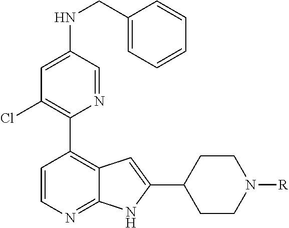

wherein R is H or —CH₃;

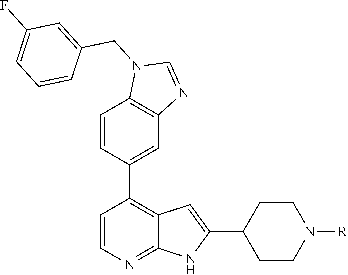

wherein R is H, C(═O)NHCH₃, or —SO₂CH₃;

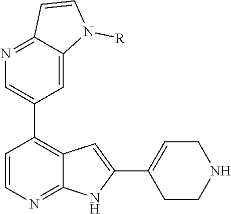

wherein R is 3-fluorobenzyl or 3-fluoropyridin-3yl;

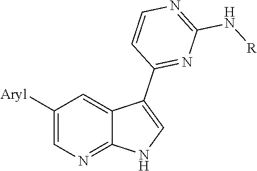

wherein Aryl is 4-fluorophenyl, 4-trifluoromethylphenyl, 3-fluorophenyl, 4-methylphenyl, 2-ethylphenyl, or 3-pyridyl and R is H, cyclopropyl, cylcopentyl, or cycloheptyl;

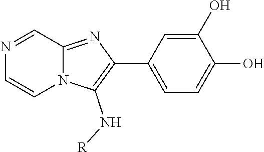

H wherein R is 2-phenylethan-1yl or (furan-2-yl)methyl;

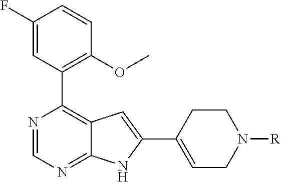

wherein R is H or —C(═O)CH₂OH;

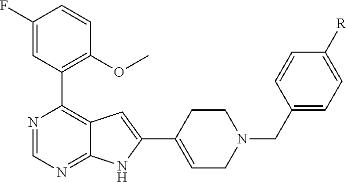

wherein R is —NHC(═O)CH₃ or —NHSO₂CH₃;

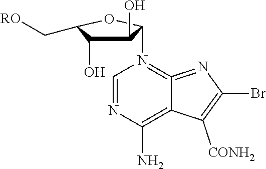

wherein R is H or isobutyl;

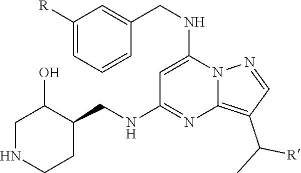

wherein R is H and R′ is —CH₃ or R is —CN and R′ is H;

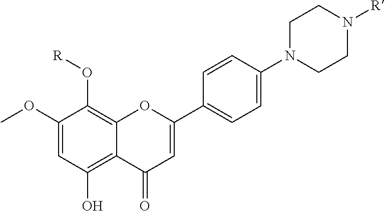

wherein R is 3,4-dimethyl-1H-pyrazol-4-yl and R′ is —CH₃ or R is piperazin-1yl and R′ is H;

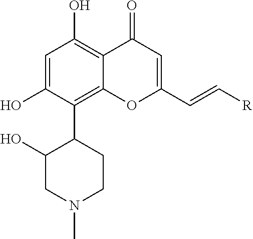

wherein R is 2,6-dichlorophenyl, 2,3,4,5,6-tetrafluorophenyl, or 3-fluorophenyl;

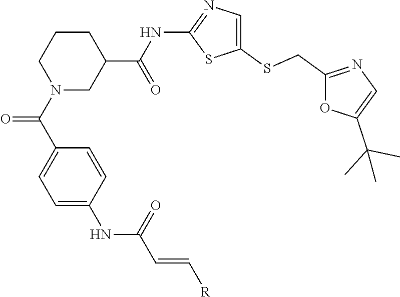

wherein R is —CH₂NCH₃ or H;

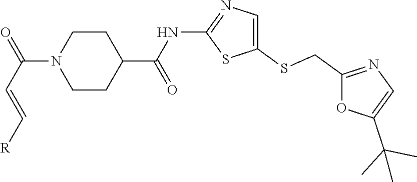

wherein R is —CH₂N(CH₃)₂ or H;

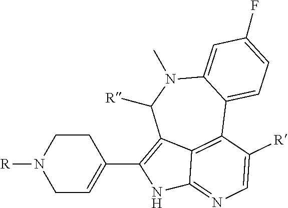

wherein R is H, —SO₂CH₃, —CH₂C(═O)N(CH₃)₂, 4-carboxylic acid-cyclobutan-1yl, or (2(hydroxymethy)pyrrolidine-1-yl)-2-one-ethan-1yl, R′ is H or F, and R″ is H or —CH₂CH₃;

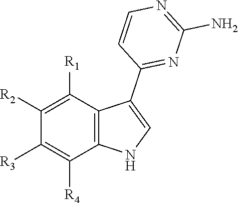

wherein R₁ is —OH, R₂ is H, R₃ is H, and R₄ is H (meridianin A), R₁ is —OH, R₂ is H, R₃ is Br, and R₄ is H (meridianin B), R₁ is H, R₂ is Br, R₃ is H, and R₄ is H (meridianin C), R₁ is H, R₂ is H, R₃ is Br, and R₄ is H (meridianin D), or R₁ is —OH, R₂ is H, R³ is H, and R₄ is Br (meridianin E); and

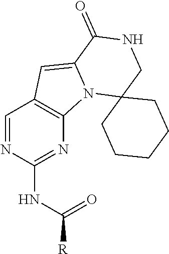

wherein R is piperidin-3yl, pyrrolodin-3yl, or morpholin-2yl.

In one embodiment, the therapeutic agent comprises a BCL2 inhibitor and a DNMT inhibitor. In one embodiment, the therapeutic agent comprises venetoclax, or a salt thereof, and 5-azacitidine, or a salt thereof.

In some embodiments, the treatments disclosed herein can include use of other drugs (e.g., antibiotics) or therapies for treating disease, e.g. MDS/AML/a type of hematopoietic cancer. For example, antibiotics can be used to treat infections and can be combined with a compound of the invention to treat disease (e.g., infections). In other embodiments, intravenous immunoglobulin (IVIG) therapy can be used as part of the treatment regime (i.e., in addition to administration of the compound(s) of the invention). For example, treatment regimens for various types of cancers can involve one or more elements selected from chemotherapy, targeted therapy, alternative therapy, immunotherapy, and the like.

Accordingly, in some embodiments, the compounds and/or compositions described herein can be used in one or more administrations to a subject, in combination with one or more BCL2 inhibitor, BTK inhibitor, chemotherapy, targeted therapy, alternative therapy, immunotherapy, DNA methyltransferase inhibitor/hypomethylating agent, anthracycline, histone deacetylase (HDAC) inhibitor, purine nucleoside analogue (antimetabolite), isocitrate dehydrogenase 1 or 2 (IDH1 and/or IDH2) inhibitor, antibody-drug conjugate, mAbs/immunotherapy, CAR-T cell therapy, Plk inhibitor, MEK inhibitor, CDK9 inhibitor, CDK8 inhibitor, retinoic acid receptor agonist, TP53 activator, smoothened receptor antagonist, ERK inhibitor, PI3K inhibitor, mTOR inhibitor, glucocorticoid receptor modulator, or EZH2 inhibitor, and the like, or one or more combinations thereof, where the compositions may be the same or different if there is more than one administration. In some embodiments, if there is more than one administration at least one composition used for at least one administration is different from the composition of at least one other administration. In one embodiment, a composition comprising a compound of Formula (I), (II), or (III) is administered to the subject separately from a composition comprising a therapeutic drug described elsewhere herein.

In particular, IRAK inhibitors have been demonstrated to have synergistic effects when administered in combination with an apoptosis modulator/inhibitor, such as a BCL2 inhibitor. As described in U.S. Patent Publication 2020/0199123 (incorporated herein by reference in its entirety), an exemplary apoptosis/BCL2 inhibitor has been shown to have a synergistic effect when used in combination with an exemplary IRAK inhibitor in multiple AMIL cell lines. Venetoclax was used as a representative apoptosis/BCL2 inhibitor.

When a concentration of an exemplary IRAK inhibitor was combined with venetoclax, the potency of venetoclax was increased by an unexpectedly high ~50-fold. According to particular aspects of the invention, this synergistic combination allows for increased efficacy of venetoclax at lower doses, to provide for avoiding at least some of the toxicity observed in the clinic. According to particular aspects, the degree of interaction is dependent on the dose ratio combination that is used, with lower concentrations of the exemplary IRAK inhibitor providing larger shifts in the venetoclax IC50. This unexpected and dramatic shift in the venetoclax IC50 is substantially more than an additive response and demonstrates the unexpected synergistic interaction of the two drugs even in cell lines that do not express activated FLT3 mutants.

Accordingly, the present invention encompasses methods for treating a disease or disorder which is responsive to inhibition of IRAK, comprising administration to a subject of a composition comprising an IRAK inhibiting compound, wherein some embodiments of the method can further involve administration of an apoptotic modulator. The apoptotic modulator may comprise a BTK and/or a BCL2 inhibitor. BTK and BCL2 inhibitors may be, for example, those known in the art. In some embodiments, the method may comprise the step of administering to the subject an apoptotic modulator. In some embodiments, the apoptotic modulator may comprise a BCL2 inhibitor selected from ABT-263 (Navitoclax), ABT-737, ABT-199 (venetoclax), GDC-0199, GX15-070 (Obatoclax) (all available from Abbott Laboratories), HA14-1, SI, 2-methoxy antimycin A3, gossypol, AT-101, apogossypol, WEHI-539, A-1155463, BXI-61, BXI-72, TW37, MIM1, UMI-77, and the like, and combinations thereof. One skilled in the art would appreciate that there are many known BCL2 inhibitors which can be used in accordance with the present invention. In some embodiments, the BCL2 inhibitor comprises venetoclax.

In some embodiments, the administration step comprises administration to a subject of a composition comprising an IRAK inhibiting compound and a BCL2 inhibitor. In some embodiments, the administration step comprises administration of a composition comprising an IRAK inhibiting compound in combination with a composition comprising a BCL2 inhibitor.

In some embodiments, the IRAK inhibiting compound is selected from Compounds 1-77, 209-214, 1a-115a, or a salt, isomer, derivative or analog thereof, and the BCL2 inhibitor is venetoclax, or a salt, isomer, derivative or analog thereof.

In some embodiments, the method can further involve administration to a subject of an immune modulator. The immune modulator can include, for example, Lenalidomide (Revlamid; Celgene Corporation). In some embodiments, the method can involve administration of an epigenetic modulator. The epigenetic modulator can include, for example, a hypomethylating agent such as azacitidine, decitabine, or a combination thereof.

In some embodiments, the compounds and/or compositions described herein can be used in one or more administrations to a subject, together with or in combination with one or more BTK inhibitors, such as, for example, ibrutinib, or a salt, isomer, derivative or analog thereof.

For example, the compounds and/or compositions described herein can be used in one or more administrations, together with or in combination with a DNA methyltransferase inhibitor/hypomethylating agent, such as, for example, azacytidine, decitabine, cytarabine (ara-C; cytosine arabinoside), and/or guadecitabine; an anthracycline, such as, for example, daunorubicin, idarubicin, doxorubicin, mitoxantrone, epirubicin, and/or CPX-351 (a combination cytarabine and daunorubicin in a fixed 5:1 molar ratio), and the like; a histone deacetylase (HDAC) inhibitor, such as, for example, vorinostat, panobinostat, valproic acid, and/or pracinostat, and the like; a purine nucleoside analogue (antimetabolite), such as, for example, fludarabine, cladribine, and/or clofarabine, and the like; an isocitrate dehydrogenase 1 or 2 (IDH1 and/or IDH2) inhibitor, such as, for example, ivosidenib (Tibsovo, for more information, see McCafferty, E. H. et al., Drugs & Therapy Perspectives, 2019, 35:160-166, which is incorporated herein by reference), AGI-6780, BAY1436032, FT-2102, IDH305, AGI-5198, ML309 (AGI-5027), GSK 321, and DC_H31, and/or enasidenib (Idhifa, for more information, see Dugan, J. et al., Expert Review of Clinical Pharmacology, 2018, 11:755-760, which is incorporated herein by reference), and the like; an antibody-drug conjugate, such as, for example, Anti-CD33 (e.g. Ac225-lintuzumab, vadastuximab, or gemtuzumab-ozogamicin) and/or Anti-CD45 (e.g. I¹³¹-apamistamab), and the like; an mAbs/Immunotherapy, such as, for example, Anti-CD70 (e.g. ARGX-110, cusatuzumab), a bispecific antibody (e.g. floteuzumab (CD123×CD3)), Anti-CTLA4 (e.g. ipilimumab), Anti-PD1/PDL1 (e.g. nivolumab, pembrolizumab, atezolizumab, avelumab, PDR001, MBG453), and/or Anti-CD47 (e.g. 5F9 (Magrolimab, for more information see Sallman, D. A. et al., Blood, 2019, 134:569, which is incorporated by reference herein)), and the like; a Plk inhibitor, such as, for example, volasertib and/or rigosertib, and the like; a MEK inhibitor, such as, for example, trametinib, cobimetinib, selumetinib, pimasertib, and/or refametinib, and the like; a CDK inhibitor such as Alvociclib, Atuveciclib, Palbociclib, Ribociclib, and/or Zotiraciclib; a CDK9 inhibitor, such as, for example, alvocidib, Bay 1143572, Dinaciclib (SCH 727965), SNS-032 (BMS-387032), TG02, CDKI-73 (LS-007), LY2857785, and/or voruciclib, and the like (for more information on CDK9 inhibitors, see Boffo, S. et al., Journal of Experimental & Clinical Cancer Research, 2018, 37:36, which is incorporated herein by reference); a CDK8 inhibitor, such as, for example, SEL120, and the like; a retinoic acid receptor agonist, such as, for example, ATRA (all-trans retinoic acid) and/or SY-1425 (a selective RARα agonist), Tamibarotene, Adapalene, Bexarotene, and the like; a TP53 activator (including a nonfunctional mutant TP53 reactivator), such as, for example, APR-246 (Eprenetapopt; for more information, see Ceder, S. et al., EMBO Mol. Med., 2021, 13:e10852, which is incorporated herein by reference), APR-548, RETRA, and/or PC14586 and the like; a CELMoD, such as Lenalidomide, Pomalidomide, CC-92480, CC-90009, Avadomide, and/or Iberdomide; a smoothened receptor antagonist, such as, for example, glasdegib, and the like; an ERK inhibitor, such as, for example, an ERK2/MAPK1 or ERK1/MAPK3 inhibitor, such as, for example, ulixertinib (for more information, see Sullivan, R. J. et al., Cancer Discovery, 2018 8:185-195, which is incorporated herein by reference), SCH772984, ravoxertinib, MK-8353, PD98059, and/or VTX-11e, and the like; a PI3K inhibitor, such as, for example, copanlisib, gedatolisib, pictilisib, fimepinostat (CUDC-907), alpelisib, leniolisib (CDZ-173), pilaralisib (XL147, SAR245408), and/or bimiralisib (PQR-309), and the like; an mTOR inhibitor, such as, for example, onatasertib, sirolimus, temsirolimus, bimiralisib (PQR-309), sapanisertib (TAK-228, INK-128), ridaforolimus (MK-8669, AP-23573), everolimus, and/or vistusertib (AZD2014), and the like; a steroid or glucocorticoid receptor modulator, such as, for example, an agonist comprising prednisolone, beclometasone, methylprednisolone, prednisone, fluticasone, budesonide, dexamethasone, and/or cortisol, and/or an antagonist comprising mifepristone, miricorilant, and/or onapristone, and/or another binding ligand comprising vamorolone (VBP15), and the like; and/or an EZH2 inhibitor, such as, for example, tazemetostat, and the like. In some embodiments, compounds and pharmaceutical compositions including the same can be used in prevention of secondary malignancies when used in combination with an EZH2 inhibitor.

In an embodiment, the compounds and/or compositions described herein can be used together with, or in combination with, a hedgehog (Hh) inhibitor, such as Daurismo (glasdegib maleate, for more information see Wolska-Washer, A. et al., Future Oncology, 2019, 15:3219-3232, which is incorporated herein by reference), Vismodegib, Erismodegib, Erivedge, Sonidegib, Odomzo, Saridegib, Exelexis, and/or Taladegib; a BCL-2 inhibitor such as venetoclax (Venclexta), navitoclax, WEHI-539, and/or A-1331852; a DNA methyltransferase inhibitor/hypomethylating agent such as decitabine (for more information, see Stresemann, C. International Journal of Cancer, 2008, 123:8-13, which is incorporated herein by reference) or Cytarabine (for more information, see Löwenberg, B. et al., N. Engl. J. Med., 2011, 364:1027-1036, which is incorporated herein by reference); a Topoisomerase I inhibitor such as Topotecan and/or Irinotecan; a Topoisomerase II inhibitor such as Mitoxantrone, Doxorubicin, and/or Daunorubicin; an aminopeptidase/Leukotriene A4 hydrolase inhibitor such as Bestatin (Ubenimex, for more information, see Hitzerd, S. M. et al., Amino Acids, 2014, 46:793-808, which is incorporated herein by reference), Ubenimex, and/or tosedostat; a FLT3/Axl/ALK inhibitor such as Xospata (Gilteritinib, for more information, see Dhillon, S., Drugs, 2019, 79:331-339, which is incorporated herein by reference) and/or ASP2215; a FLT3/KIT/PDGFR, PKC, and/or KDR inhibitor such as Rydapt (Midostaurin, for more information, see Sheridan, C., Nature Biotechnology, 2017, 35:696-698, which is incorporated herein by reference); a Syk inhibitor such as fostamatinib (R788), entospletinib (GS-9973, for more information, see Walker, A. R. et al., Blood, 2016, 128:2831, which is incorporated by reference herein), cerdulatinib (PRT062070), and/or TAK-659; an E-selectin inhibitor such as Uproleselan (for more information, see Barbier, V. et al., Nature Commun., 2020, 11:2042); an NEDD8-activator such as Pevonedistat (for more information, see Swords, R. T. et al., British J. Haematology, 2015, 169: 534-543, which is incorporated by reference herein); an MDM2 inhibitor such as idasanutlin (for more information, see Lehmann, C. et al., Journal of Hematology & Oncology, 2016, 9:50, which is incorporated by reference herein), AMG-232, and/or CGM-097; a PLK1 inhibitor such as Onvansertib, BI2536, and/or Volasertib (for more information, see Van den Bossche, J. et al., Medicinal Research Reviews, 2016, 36:749-786, which is incorporated herein by reference); an Aura A inhibitor such as Alisertib (MLN8237; for more information, see Goldberg, S. L. et al., Leukemia Research Reports, 2014, 3:58-61, which is incorporated by reference herein), MLN8054, TAS-119, and/or erbumine (LY3295668); an aurora kinase inhibitor such as Alisertib, Danusertib, Barasertib, and/or Ilorasertib; an EGFR inhibitor such as Erlotinib, Dacomitinib, and/or Varlitinib; an AuroraB/C/VEGFR1/2/3/FLT3/CSF-1R/Kit/PDGFRA/B inhibitor such as Ilorasertib (ABT-348; for more information, see Garcia-Manero, G. et al., Investigational New Drugs, 2015, 33:870-880, which is incorporated by reference herein); an AKT 1, 2, and/or 3 inhibitor such as Uprosertib (for more information, see Darici, S. et al., J. Clin. Med., 2020, 9:2934, which is incorporated by reference herein), Afuresertib (GSK2110183), CCT128930, Miransertib (ARQ 092), Capivasertib (AZD5363), GSK690693, Ipatasertib (GDC-0068), BAY1125976, and/or Oridonin (NSC-250682); a ABL1/2/SRC/EPHA2/LCK/YES1/KIT/PDGFRB/FYN inhibitor such as Dasatinib; a farnesyltransferase inhibitor such as tipifarnib (for more information, see Epling-Burnette, P. K. et al., Expert Opinion on Investigational Drugs, 2010, 19:689-698, which is incorporated by reference herein), lonafarnib, manumycin A, gingerol, gliotoxin, and/or a-hydroxy farnesyl phosphoric acid; a BRAF/MAP2K1/MAP2K2 inhibitor such as Trametinib; a Menin-KMT2A/MLL inhibitor such as Ko-539 and/or SNDX-5613 (for more information on Ko-539 and SNDX-5613, see Gundry, M. C. et al., Cancer Cell, 2020, 37:267-269, which is incorporated by reference herein); an anti-metabolite such as Cytarabine, Floxuridine, 5-Fluorouracil, Prexasertib, Raltitrexed, and/or Methotrexate; and/or a multikinase inhibitor such as Dasatinib.

In one embodiment, the compounds and/or compositions described herein are used in one or more administrations, together with or in combination with Lenalidomide which is a highly effective treatment for myelodysplastic syndrome (MDS) with deletion of chromosome 5q (del(5q)). Lenalidomide induces the ubiquitination of casein kinase 1A1 (CK1α) by the E3 ubiquitin ligase CUL4-RBX1-DDB1-CRBN (known as CRL4CRBN), resulting in CK1α degradation. CK1α is encoded by a gene within the common deleted region for del(5q) MDS and haploinsufficient expression sensitizes cells to lenalidomide therapy, providing a mechanistic basis for the therapeutic window of lenalidomide in del(5q) MDS. In one embodiment, the compounds and/or compositions described herein are used in one or more administrations, together with or in combination with Cytarabine (ara-C, cytosine arabinoside), which has been used for the treatment of acute myeloid leukemia (AML) for more than three decades. It was initially used in remission-induction therapy at a dose of 100 to 200 mg per square meter of body-surface area. From about 1975 to 1985, investigators began evaluating the use of high-dose cytarabine therapy, given in a dose of 3000 mg per square meter twice daily for 6 days. In single-group studies, high response rates were noted among patients with relapse and promising results were reported for those with a new diagnosis of AML. However, more recent studies have demonstrated that induction therapy with cytarabine at lower dosages already produces maximal antileukemic effects for all response end points, suggesting a plateau in the dose-response relationship above this dose level and thus suggesting that high-dose cytarabine results in excessive toxic effects without therapeutic benefit. In one embodiment, the compounds and/or compositions described herein are used in one or more administrations, together with or in combination with a hypomethylating agent such as Azacitidine, Decitabine and/or Venclexta. DNA methylation is the modification of DNA nucleotides by addition of a methyl group. A hypomethylating agent (or demethylating agent) is a drug that inhibits DNA methylation. Because DNA methylation affects cellular function through successive generations of cells without changing the underlying DNA sequence, hypomethylating agents are considered a type of epigenetic therapy. Currently available hypomethylating agents block the activity of DNA methyltransferase (DNA methyltransferase inhibitors/DNMT inhibitors). Two members of the class, azacitidine and decitabine, are FDA-approved for use in the United States in myelodysplastic syndrome. Azacitidine, marketed as Vidaza, is used mainly in the treatment of myelodysplastic syndrome, for which it received approval by the U.S. Food and Drug Administration (FDA) on May 19, 2004. In two randomized controlled trials comparing azacitidine to supportive treatment, 16% of subjects with myelodysplastic syndrome who were randomized to receive azacitidine had a complete or partial normalization of blood cell counts and bone marrow morphology, compared to none who received supportive care, and about two-thirds of patients who required blood transfusions no longer needed them after receiving azacitidine. Azacitidine can also be used for the treatment of acute myeloid leukemia as a hypomethylating agent. Decitabine has shown significant clinical benefits in the treatment of myelodysplastic syndrome (MDS) by depleting DNA methyltransferase enzymes and inducing DNA demethylation and epigenetic reprogramming in vitro. Venclexta is a selective small-molecule inhibitor of BCL-2, an antiapoptotic protein. The overexpression of BCL-2 in cancer cells is associated with tumor-cell survival and resistance to chemotherapy. Therefore, BCL-2 inhibitors such as Venclexta facilitate apoptosis by binding directly to the BCL-2 protein, displacing proapoptotic proteins, and triggering mitochondrial outer-membrane permeabilization and caspase activation. In one embodiment, the compounds and/or compositions described herein are used in one or more administrations, together with or in combination with an anti-CD47 Monoclonal Antibody such as Magrolimab. Monoclonal antibodies against CD47 are designed to interfere with recognition of CD47 by the SIRPα receptor on macrophages, thus blocking the “don't eat me” signal used by cancer cells to avoid being ingested by macrophages. Magrolimab is a first-in-class investigational monoclonal antibody against CD47 and macrophage checkpoint inhibitor which is being developed in several hematologic and solid tumor malignancies, including MDS. Magrolimab has been granted Fast Track Designation by the FDA for the treatment of MDS, AML, diffuse large B-cell lymphoma (DLBCL) and follicular lymphoma. In one embodiment, the compounds and/or compositions described herein are used in one or more administrations, together with or in combination with an SYK inhibitor such as Entospletinib. Spleen tyrosine kinase (SYK) is a nonreceptor cytoplasmic tyrosine kinase primarily expressed in cells of hematopoietic lineage. Constitutive activation of SYK in AML has been reported and targeted inhibition of SYK induced differentiation in vitro and demonstrated anti-leukemia activity in AML mouse models. SYK has also been shown to directly phosphorylate the FLT3 receptor, modulating its activation and possibly promoting its role in leukemogenesis. Entospletinib is an orally bioavailable, selective inhibitor of SYK shown to be clinically active in B-cell malignancies. In one embodiment, the compounds and/or compositions described herein are used in one or more administrations, together with or in combination with an E-selectin inhibitor such as Uproleselan. E-selectin directly triggers signaling pathways that promote malignant cell survival and regeneration. Using acute AML mouse models, it was shown that AML blasts release inflammatory mediators that upregulate endothelial niche E-selectin expression. Alterations in cell-surface glycosylation associated with oncogenesis enhances AML blast binding to E-selectin and enable promotion of pro-survival signaling through AKT/NF-κB pathways. In vivo AML blasts with highest E-selectin binding potential are 12-fold more likely to survive chemotherapy and main contributors to disease relapse. Therapeutic blockade of E-selectin using small molecule mimetic Uproleselan effectively inhibits this niche-mediated pro-survival signaling, dampens AML blast regeneration, and strongly synergizes with chemotherapy, doubling the duration of mouse survival over chemotherapy alone. In one embodiment, the compounds and/or compositions described herein are used in one or more administrations, together with or in combination with a CDK9 inhibitor such as Alvocidib. The cyclin-dependent kinase 9 (CDK9) pathway is dysregulated in AML and therefore targeting this pathway is an attractive approach to treat AML. Inhibition of CDK9 leads to downregulation of cell survival genes regulated by super enhancers such as MCL-1, MYC, and cyclin D1. As CDK9 inhibitors are nonselective, predictive biomarkers that may help identify patients most likely to respond to CDK9 inhibitors are now being utilized, with the goal of improving efficacy and safety. Alvocidib is a multi-serine threonine cyclin-dependent kinase inhibitor with demonstrable in vitro and clinical activity in AML when combined in a timed sequential chemotherapy regimen. In one embodiment, the compounds and/or compositions described herein are used in one or more administrations, together with or in combination with a Menin-KMT2A (MLL) inhibitor such as Ko-539 and/or SNDX-5613. When overexpressed in murine hematopoietic progenitors, Meningioma-1 (MN1) causes an aggressive AML characterized by an aberrant myeloid precursor-like gene expression program that shares features of KMT2A-rearranged (KMT2A-r) leukemia, including high levels of Hoxa and Meis1 gene expression. Menin (Men1) is also critical for the self-renewal of MN1-driven AML through the maintenance of a distinct gene expression program. Genetic inactivation of Men1 led to a decrease in the number of functional leukemia-initiating cells. Pharmacologic inhibition of the KMT2A-Menin interaction has been shown to decrease colony-forming activity, induce differentiation programs in MN1-driven murine leukemia, and decrease leukemic burden in a human AML xenograft. These results nominate Menin inhibition as a promising therapeutic strategy in MN1-driven leukemia. A phase 2 clinical trial of SNDX-5613 will recruit patients according to disease and molecular genetics (MLLr AML, NPMlc AML, or MLLr acute lymphoid leukemia) while KO-539 is recruiting patients for a phase 1 study for relapsed/refractory AML. Both compounds showed excellent pharmacokinetic properties and low toxicity profiles in pre-clinical studies. In one embodiment, the compounds and/or compositions described herein are used in one or more administrations, together with or in combination with a nonfunctional mutant TP53 reactivator such as Eprenetapopt (APR-246). TP53 gene mutations are detected in approximately 10%-20% of patients with de novo myelodysplastic syndromes (MDS) or acute myeloid leukemia (AMIL) and 30%-40% of patients with therapy-related disease. Treatment outcomes for patients with TP53 mutations are poor with available therapies. Hypomethylating agents (HMAs), such as azacitidine and decitabine, yield statistically similar complete remission (CR) rates of approximately 15%-20% in patients with either TP53-mutant or wild-type MDS. However, remissions in TP53-mutant patients are brief with a median overall survival (OS) ranging from 5 to 12 months reflecting the significant unmet medical need for targeted therapies for patients with TP53-mutant MDS and AML. Eprenetapopt (APR-246) is converted to methylene quinuclidinone (MQ) that targets mutant p53 protein and perturbs cellular antioxidant balance. APR-246 is currently being tested in a phase III clinical trial in myelodysplastic syndrome (MDS).

In some embodiments, the one or more therapeutic agents can be in the form of salts, optical and geometric isomers, and salts of isomers. In other embodiments, the therapeutic agent can be in various forms, such as uncharged molecules, components of molecular complexes, or non-irritating pharmacologically acceptable salts, including but not limited to hydrochloride, hydrobromide, sulphate, phosphate, nitrate, borate, acetate, maleate, tartrate, and salicylate. In some instances, for acidic compounds, salts can include metals, amines, or organic cations (e.g. quaternary ammonium). In yet other embodiments, simple derivatives of the therapeutic agents (e.g., ethers, esters, or amides) which have desirable retention and release characteristics but which are easily hydrolyzed by body pH, enzymes, or other suitable means, can be employed.

In some embodiments, the therapeutic agent has a chiral center and can exist in and be isolated in optically active and racemic forms. In other embodiments, the therapeutic agent may exhibit polymorphism. Some embodiments of the present disclosure encompass any racemic, optically active, polymorphic, or stereoisomeric form, or mixtures thereof, of a compound described herein, including isotopically-labeled and radio-labeled compounds. See e.g., Goding, 1986, Monoclonal Antibodies Principles and Practice; Academic Press, p. 104. Such isomers can be isolated by standard resolution techniques, including e.g., fractional crystallization, chiral chromatography, and the like. See e.g., Eliel, E. L. & Wilen S. H., 1993, Stereochemistry in Organic Compounds; John Wiley & Sons, New York. The preparation of optically active forms can be accomplished by any suitable method, including but not limited to, resolution of the racemic form by recrystallization techniques, synthesis from optically-active starting materials, chiral synthesis, or chromatographic separation using a chiral stationary phase.

In some embodiments, the therapeutic agent has asymmetric centers and can occur as racemates, racemic mixtures, and as individual enantiomers or diastereoisomers, with all isomeric forms as well as mixtures thereof being contemplated for use in the compounds and methods described herein. The compounds contemplated for use in the compounds and methods described herein do not include those that are known in the art to be too unstable to synthesize and/or isolate.

The therapeutic agents disclosed herein can also contain unnatural proportions of atomic isotopes at one or more of the atoms that constitute such compounds. For example, the compounds can be radiolabeled with radioactive isotopes, such as for example tritium (³H), iodine-125 (¹²⁵I), or carbon-14 (¹⁴C). All isotopic variations of the compounds disclosed herein, whether radioactive or not, are encompassed within the contemplated scope.

In some embodiments, metabolites of the therapeutic agents disclosed herein are useful for the methods disclosed herein.

In some embodiments, the therapeutic agents contemplated herein may be provided in the form of a prodrug. The term “prodrug” refers to a compound that can be converted into a compound (e.g., a biologically active compound) described herein in vivo. Prodrugs can be useful for a variety of reason known in the art, including e.g., ease of administration due e.g., to enhanced bioavailability in oral administration, and the like. The prodrug can also have improved solubility in pharmaceutical compositions over the biologically active compounds. An example, without limitation, of a prodrug is a compound which is administered as an ester (i.e., the “prodrug”) to facilitate transmittal across a cell membrane where water solubility is detrimental to mobility but which then is metabolically hydrolyzed to the carboxylic acid, the active entity, once inside the cell where water solubility is beneficial. Conventional procedures for the selection and preparation of suitable prodrug derivatives are described, for example, in Design of Prodrugs, (ed. H. Bundgaard, Elsevier, 1985), which is hereby incorporated herein by reference for the limited purpose describing procedures and preparation of suitable prodrug derivatives.

Certain the therapeutic agent disclosed herein can exist in unsolvated forms as well as solvated forms, including hydrated forms. In general, the solvated forms are equivalent to unsolvated forms and are encompassed within the scope of contemplated compounds. Certain the therapeutic agents of the present disclosure can exist in multiple crystalline or amorphous forms. In general, all physical forms are equivalent for the compounds and methods contemplated herein and are intended to be within the scope disclosed herein.

Further therapies are described below and are contemplated in combination therapies in the context of the present invention.

### Chemotherapy/Targeted Therapy/Alternative Therapy

Cancers are commonly treated with chemotherapy and/or targeted therapy and/or alternative therapy. Chemotherapies act by indiscriminately targeting rapidly dividing cells, including healthy cells as well as tumor cells, whereas targeted cancer therapies rather act by interfering with specific molecules, or molecular targets, which are involved in cancer growth and progression. Targeted therapy generally targets cancer cells exclusively, having minimal damage to normal cells. Chemotherapies and targeted therapies which are approved and/or in the clinical trial stage are known to those skilled in the art. Any such compound can be utilized in the practice of the present invention.

For example, approved chemotherapies include abitrexate (Methotrexate Injection), abraxane (Paclitaxel Injection), adcetris (Brentuximab Vedotin Injection), adriamycin (Doxorubicin), adrucil Injection (5-FU (fluorouracil)), afinitor (Everolimus), afinitor Disperz (Everolimus), alimta (PEMETREXED), alkeran Injection (Melphalan Injection), alkeran Tablets (Melphalan), aredia (Pamidronate), arimidex (Anastrozole), aromasin (Exemestane), arranon (Nelarabine), arzerra (Ofatumumab Injection), avastin (Bevacizumab), beleodaq (Belinostat Injection), bexxar (Tositumomab), BiCNU (Carmustine), blenoxane (Bleomycin), blincyto (Blinatumoma b Injection), bosulif (Bosutinib), busulfex Injection (Busulfan Injection), campath (Alemtuzumab), camptosar (Irinotecan), caprelsa (Vandetanib), casodex (Bicalutamide), CeeNU (Lomustine), CeeNU Dose Pack (Lomustine), cerubidine (Daunorubicin), clolar (Clofarabine Injection), cometriq (Cabozantinib), cosmegen (Dactinomycin), cotellic (Cobimetinib), cyramza (Ramucirumab Injection), cytosarU (Cytarabine), cytoxan (Cytoxan), cytoxan Injection (Cyclophosphamide Injection), dacogen (Decitabine), daunoXome (Daunorubicin Lipid Complex Injection), decadron (Dexamethasone), depoCyt (Cytarabine Lipid Complex Injection), dexamethasone Intensol (Dexamethasone), dexpak Taperpak (Dexamethasone), docefrez (Docetaxel), doxil (Doxorubicin Lipid Complex Injection), droxia (Hydroxyurea), DTIC (Decarbazine), eligard (Leuprolide), ellence (Ellence (epirubicin)), eloxatin (Eloxatin (oxaliplatin)), elspar (Asparaginase), emeyt (Estramustine), erbitux (Cetuximab), erivedge (Vismodegib), erwinaze (Asparaginase Erwinia chrysanthemi), ethyol (Amifostine), etopophos (Etoposide Injection), eulexin (Flutamide), fareston (Toremifene), farydak (Panobinostat), faslodex (Fulvestrant), femara (Letrozole), firmagon (Degarelix Injection), fludara (Fludarabine), folex (Methotrexate Injection), folotyn (Pralatrexate Injection), FUDR (FUDR (floxuridine)), gazyva (Obinutuzumab Injection), gemzar (Gemcitabine), gilotrif (Afatinib), gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Ibrance (Palbociclib), Iclusig (Ponatinib), Idamycin PFS (Idarubicin), Ifex (Ifosfamide), Imbruvica (Ibrutinib), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Keytruda (Pembrolizumab Injection), Kyprolis (Carfilzomib), Lanvima (Lenvatinib), Leukeran (Chlorambucil), Leukine (Sargramostim), Leustatin (Cladribine), Lonsurf (Trifluridine and Tipiracil), Lupron (Leuprolide), Lupron Depot (Leuprolide), Lupron DepotPED (Leuprolide), Lynparza (Olaparib), Lysodren (Mitotane), Marqibo Kit (Vincristine Lipid Complex Injection), Matulane (Procarbazine), Megace (Megestrol), Mekinist (Trametinib; for more information, see Borthakur, G. et al., Blood, 2012, 120:677, which is incorporated by reference herein), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin, for more information, see Norsworthy, K. J. et al., Oncologist, 2018, 23:1103-1108, which is incorporated herein by reference), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone, for more information, see Fox, E. J., Neurology, 2004, 28(12 Suppl 6):S15-8, which is incorporated herein by reference), Odomzo (Sonidegib), Oncaspar (Pegaspargase), Oncovin (Vincristine), Ontak (Denileukin Diftitox), onxol (Paclitaxel Injection), opdivo (Nivolumab Injection), panretin (Alitretinoin), paraplatin (Carboplatin), perj eta (Pertuzumab Injection), platinol (Cisplatin), platinol (Cisplatin Injection), platinolAQ (Cisplatin), platinolAQ (Cisplatin Injection), pomalyst (Pomalidomide), prednisone Intensol (Prednisone), proleukin (Aldesleukin), purinethol (Mercaptopurine), reclast (Zoledronic acid), revlimid (Lenalidomide; for more information see Kr6nke, J. et al., Nature, 2015, 523:183-188, which is incorporated by reference herein), actimid (Pomalidomid), rheumatrex (Methotrexate), rituxan (Rituximab), roferonA alfaa (Interferon alfa-2a), rubex (Doxorubicin), sandostatin (Octreotide), sandostatin LAR Depot (Octreotide), soltamox (Tamoxifen), sprycel (Dasatinib; for more information, see Duong, V. H. et al., Leukemia Research, 2013, 37:300-304, which is incorporated herein by reference), sterapred (Prednisone), sterapred DS (Prednisone), stivarga (Regorafenib), supprelin LA (Histrelin Implant), sutent (Sunitinib), sylatron (Peginterferon Alfa-2b Injection (Sylatron)), sylvant (Siltuximab Injection), synribo (Omacetaxine Injection), tabloid (Thioguanine), taflinar (Dabrafenib), tarceva (Erlotinib), targretin Capsules (Bexarotene), tasigna (Decarbazine), taxol (Paclitaxel Injection), taxotere (Docetaxel), temodar (Temozolomide), temodar (Temozolomide Injection), tepadina (Thiotepa), thalomid (Thalidomide), theraCys BCG (BCG), thioplex (Thiotepa), TICE BCG (BCG), toposar (Etoposide Injection), torisel (Temsirolimus), treanda (Bendamustine hydrochloride), trelstar (Triptorelin Injection), trexall (Methotrexate), trisenox (Arsenic trioxide), tykerb (lapatinib), unituxin (Dinutuximab Injection), valstar (Valrubicin Intravesical), vantas (Histrelin Implant), vectibix (Panitumumab), velban (Vinblastine), velcade (Bortezomib), vepesid (Etoposide), vepesid (Etoposide Injection), vesanoid (Tretinoin), vidaza (Azacitidine), vincasar PFS (Vincristine), vincrex (Vincristine), votrient (Pazopanib), vumon (Teniposide), wellcovorin IV (Leucovorin Injection), xalkori (Crizotinib), xeloda (Capecitabine), xtandi (Enzalutamide), yervoy (Ipilimumab Injection), yondelis (Trabectedin Injection), zaltrap (Ziv-aflibercept Injection), zanosar (Streptozocin), zelboraf (Vemurafenib), zevalin (Ibritumomab Tiuxetan), zoladex (Goserelin), zolinza (Vorinostat), zometa (Zoledronic acid), zortress (Everolimus), zydelig (Idelalisib), zykadia (Ceritinib), zytiga (Abiraterone), and the like, in addition to analogs and derivatives thereof. For example, approved targeted therapies include ado-trastuzumab emtansine (Kadcyla), afatinib (Gilotrif), aldesleukin (Proleukin), alectinib (Alecensa), alemtuzumab (Campath), axitinib (Inlyta), bosutinib (Bosulif), brentuximab vedotin (Adcetris), cabozantinib (Cabometyx [tablet], Cometriq [capsule]), canakinumab (Ilaris), carfilzomib (Kyprolis), ceritinib (Zykadia), cetuximab (Erbitux), cobimetinib (Cotellic), crizotinib (Xalkori), dabrafenib (Tafinlar), daratumumab (Darzalex), dasatinib (Sprycel), denosumab (Xgeva), dinutuximab (Unituxin), elotuzumab (Empliciti), erlotinib (Tarceva, for more information, see Boehrer, S. et al., Blood, 2008, 111:2170-2180, which is incorporated by reference herein), everolimus (Afinitor), gefitinib (Iressa), ibritumomab tiuxetan (Zevalin), ibrutinib (Imbruvica), idelalisib (Zydelig), imatinib (Gleevec), ipilimumab (Yervoy), ixazomib (Ninlaro), lapatinib (Tykerb), lenvatinib (Lenvima), necitumumab (Portrazza), nilotinib (Tasigna), nivolumab (Opdivo), obinutuzumab (Gazyva), ofatumumab (Arzerra, HuMax-CD20), olaparib (Lynparza),osimertinib (Tagrisso), palbociclib (Ibrance), panitumumab (Vectibix), panobinostat (Farydak), pazopanib (Votrient), pembrolizumab (Keytruda), pertuzumab (Perjeta), ponatinib (Iclusig), ramucirumab (Cyramza), rapamycin, regorafenib (Stivarga), rituximab (Rituxan, Mabthera), romidepsin (Istodax), ruxolitinib (Jakafi), siltuximab (Sylvant), sipuleucel-T (Provenge), sirolimus, sonidegib (Odomzo), sorafenib (Nexavar), sunitinib, tamoxifen, temsirolimus (Torisel), tocilizumab (Actemra), tofacitinib (Xeljanz), tositumomab (Bexxar), trametinib (Mekinist), trastuzumab (Herceptin), vandetanib (Caprelsa), vemurafenib (Zelboraf), venetoclax (Venclexta), vismodegib (Erivedge), vorinostat (Zolinza), ziv-aflibercept (Zaltrap), and the like, in addition to analogs and derivatives thereof. In an embodiment, the approved chemotherapy is an anthracycline, such as Doxorubicen, Daunarubicin, Epirubicin, and/or Idarubicin. In one embodiment, the approved chemotherapy is selected from Azacitidine (for more information, see Keating, G. M., Drugs, 2012, 72:1111-1136, which is incorporated herein by reference), Venclexta (for more information, see Raedler, L. A., Journal of Hematology Oncology Pharmacy, 2017, 7:53-55, which is incorporated herein by reference)

Those skilled in the art can determine appropriate chemotherapy and/or targeted therapy and/or alternative therapy options, including treatments that have been approved and those that in clinical trials or otherwise under development. Some targeted therapies are also immunotherapies. Any relevant chemotherapy, target therapy, and alternative therapy treatment strategies can be utilized, alone or in combination with one or more additional cancer therapy, in the practice of the present invention.

### Immunotherapy

In some embodiments, immunotherapies include cell-based immunotherapies, such as those involving cells which effect an immune response (such as, for example, lymphocytes, macrophages, natural killer (NK) cells, dendritic cells, cytotoxic T lymphocytes (CTL), antibodies and antibody derivatives (such as, for example, monoclonal antibodies, conjugated monoclonal antibodies, polyclonal antibodies, antibody fragments, radiolabeled antibodies, chemolabeled antibodies, etc.), immune checkpoint inhibitors, vaccines (such as, for example, cancer vaccines (e.g. tumor cell vaccines, antigen vaccines, dendritic cell vaccines, vector-based vaccines, etc.), e.g. oncophage, sipuleucel-T, and the like), immunomodulators (such as, for example, interleukins, cytokines, chemokines, etc.), topical immunotherapies (such as, for example, imiquimod, and the like), injection immunotherapies, adoptive cell transfer, oncolytic virus therapies (such as, for example, talimogene laherparepvec (T-VEC), and the like), immunosuppressive drugs, helminthic therapies, other non-specific immunotherapies, and the like. Immune checkpoint inhibitor immunotherapies are those that target one or more specific proteins or receptors, such as PD-1, PD-L1, CTLA-4, and the like. Immune checkpoint inhibitor immunotherapies include ipilimumab (Yervoy), nivolumab (Opdivo), pembrolizumab (Keytruda), and the like. Non-specific immunotherapies include cytokines, interleukins, interferons, and the like. In some embodiments, an immunotherapy assigned or administered to a subject can include an interleukin, and/or interferon (IFN), and/or one or more suitable antibody-based reagent, such as denileukin diftitox and/or administration of an antibody-based reagent selected from the group consisting of ado-trastuzumab emtansine, alemtuzumab, atezolizumab, bevacizumab, blinatumomab, brentuximab vedotin, cetuximab, catumaxomab, gemtuzumab, ibritumomab tiuxetan, ipilimumab, natalizumab, nimotuzumab, nivolumab, ofatumumab, panitumumab, pembrolizumab, rituximab, tositumomab, trastuzumab, vivatuxin, and the like. In some embodiments, an immunotherapy assigned or administered to a subject can include an indoleamine 2,3-dioxygenase (IDO) inhibitor, adoptive T-cell therapy, virotherapy (T-VEC), and/or any other immunotherapy whose efficacy extensively depends on anti-tumor immunity.

Those skilled in the art can determine appropriate immunotherapy options, including treatments that have been approved and those that in clinical trials or otherwise under development. Any relevant immunotherapy treatment strategies, alone or in combination with one or more additional cancer therapy, can be utilized in the practice of the present invention.

### Other Cancer Treatments

In addition to chemotherapies, targeted therapies, alternative therapies, and immunotherapies, cancer can additionally be treated by other strategies. These include surgery, radiation therapy, hormone therapy, stem cell transplant, precision medicine, and the like; such treatments and the compounds and compositions utilized therein are known to those skilled in the art. Any such treatment strategies can be utilized in the practice of the present invention.

Alternative treatment strategies have also been used with various types of cancers. Such treatment can be used alone or in combination with any other treatment modality. These include exercise, massage, relaxation techniques, yoga, acupuncture, aromatherapy, hypnosis, music therapy, dietary changes, nutritional and dietary supplements, and the like; such treatments are known to those skilled in the art. Any such treatment strategies can be utilized, alone or in combination with one or more additional cancer therapy, in the practice of the present invention.

### Dosage and Administration Routes

Other embodiments of the invention can include methods of administering or treating an animal, which can involve treatment with an amount of at least one compound of the invention (e.g., Formula (I)) that is effective to treat the disease, condition, or disorder that the organism has, or is suspected of having, or is susceptible to, or to bring about a desired physiological effect. In some embodiments, the composition or pharmaceutical composition comprises at least one compound of the invention (e.g., Formula (I), (II), (III), (V), (VI), (VII), (Ja-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)) which can be administered to an animal (e.g., mammals, primates, monkeys, or humans) in an amount of about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight or about 6.5 mg/kg human body weight. In some instances, some subjects (e.g., mammals, mice, rabbits, feline, porcine, or canine) can be administered a dosage of about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. Of course, those skilled in the art will appreciate that it is possible to employ many concentrations in the methods of the present invention, and using, in part, the guidance provided herein, will be able to adjust and test any number of concentrations in order to find one that achieves the desired result in a given circumstance. In some embodiments, a dose or a therapeutically effective dose of a compound disclosed herein will be that which is sufficient to achieve a plasma concentration of the compound or its active metabolite(s) within a range set forth herein, e.g., about 1-10 nM, 10-100 nM, 0.1-1 μM, 1-10 μM, 10-100 μM, 100-200 μM, 200-500 μM, or even 500-1000 μM, preferably about 1-10 nM, 10-100 nM, or 0.1-1 μM. Without wishing to be bound by any theory, it is believed that such compounds are indicated in the treatment or management of hematopoietic cancers, such as, for example, MDS and/or AML and/or DLBCL, etc., as described herein.

In other embodiments, the compounds and/or pharmaceutical compounds of the invention (e.g., compounds of Formula (I), (II), or (III) and pharmaceutical compositions including the same) can be administered in combination with one or more other therapeutic agents for a given disease, condition, or disorder.

The compounds and pharmaceutical compositions are preferably prepared and administered in dose units. Solid dose units are tablets, capsules and suppositories. For treatment of a subject, depending on activity of the compound, manner of administration, nature and severity of the disease or disorder, age and body weight of the subject, different daily doses can be used.

Under certain circumstances, however, higher or lower daily doses can be appropriate. The administration of the daily dose can be carried out both by single administration in the form of an individual dose unit or else several smaller dose units and also by multiple administrations of subdivided doses at specific intervals.

The compounds and pharmaceutical compositions contemplated herein can be administered locally or systemically in a therapeutically effective dose. Amounts effective for this use will, of course, depend on the severity of the disease or disorder and the weight and general state of the subject. Typically, dosages used in vitro can provide useful guidance in the amounts useful for in situ administration of the pharmaceutical composition, and animal models can be used to determine effective dosages for treatment of particular disorders.

Various considerations are described, e. g., in Langer, 1990, Science, 249: 1527; Goodman and Gilman's (eds.), 1990, Id., each of which is herein incorporated by reference and for all purposes. Dosages for parenteral administration of active pharmaceutical agents can be converted into corresponding dosages for oral administration by multiplying parenteral dosages by appropriate conversion factors. As to general applications, the parenteral dosage in mg/mL times 1.8=the corresponding oral dosage in milligrams (“mg”). As to oncology applications, the parenteral dosage in mg/mL times 1.6=the corresponding oral dosage in mg. An average adult weighs about 70 kg. See e.g., Miller-Keane, 1992, Encyclopedia & Dictionary of Medicine, Nursing & Allied Health, 5th Ed., (W. B. Saunders Co.), pp. 1708 and 1651.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In some embodiments, the compounds and/or pharmaceutical compositions can include a unit dose of one or more compounds of the invention (e.g., compounds of Formula (I), (II), or (III) and pharmaceutical compositions including the same) in combination with a pharmaceutically acceptable carrier and, in addition, can include other medicinal agents, pharmaceutical agents, carriers, adjuvants, diluents, and excipients. In certain embodiments, the carrier, vehicle or excipient can facilitate administration, delivery and/or improve preservation of the composition. In other embodiments, the one or more carriers, include but are not limited to, saline solutions such as normal saline, Ringer's solution, PBS (phosphate-buffered saline), and generally mixtures of various salts including potassium and phosphate salts with or without sugar additives such as glucose. Carriers can include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In other embodiments, the one or more excipients can include, but are not limited to water, saline, dextrose, glycerol, ethanol, or the like, and combinations thereof. Nontoxic auxiliary substances, such as wetting agents, buffers, or emulsifiers may also be added to the composition. Oral formulations can include such normally employed excipients as, for example, pharmaceutical grades of mannitol, lactose, starch, magnesium stearate, sodium saccharine, cellulose, and magnesium carbonate.

The quantity of active component in a unit dose preparation can be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The compounds of the invention (e.g., compounds according to Formula (I), (II), or (III)) can be administered to subjects by any number of suitable administration routes or formulations. The compounds of the invention (e.g., Formula (I), (II), (III), (V), (VI), (VII), (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)) of the invention can also be used to treat subjects for a variety of diseases. Subjects include but are not limited to mammals, primates, monkeys (e.g., macaque, rhesus macaque, or pig tail macaque), humans, canine, feline, bovine, porcine, avian (e.g., chicken), mice, rabbits, and rats. As used herein, the term “subject”, unless stated otherwise, encompasses both human and non-human subjects.

The route of administration of the compounds of the invention (e.g., Formula (I)) can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, subcutaneous administration, topical administration, intradermal administration, oral administration, sublingual administration, intranasal administration, or intramuscular administration. The choice of administration route can depend on the compound identity (e.g., the physical and chemical properties of the compound) as well as the age and weight of the animal, the particular disease (e.g., cancer or MDS), and the severity of the disease (e.g., stage or severity of cancer or MDS). Of course, combinations of administration routes can be administered, as desired.

Some embodiments of the invention include a method for providing a subject with a composition comprising one or more compounds of the invention (e.g., Formula (I)) described herein (e.g., a pharmaceutical composition) which comprises one or more administrations of one or more such compositions; the compositions may be the same or different if there is more than one administration.

### Methods of Increasing Survivability of a Subject

In yet another aspect, the present disclosure provides a method of increasing survivability in a subject diagnosed with acute myeloid leukemia (AML) or suspected of having AML, the method comprising administering to the subject a therapeutically effective amount of a compound of Formula (I), (II), (III), (V), (VI), or (VII) (including a compound of Formula (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)) or a salt, ester, solvate, optical isomer, geometric isomer, or salt of an isomer thereof or a composition comprising a compound of Formula (I), (II), (III), (V), (VI), or (VII) (including a compound of Formula (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)), or a salt, ester, solvate, optical isomer, geometric isomer, or salt of an isomer thereof. In one embodiment, the survivability of the subject is increased compared to a subject treated with a therapeutically effective amount of the standard of care for AML. In one embodiment, the standard of care for AML comprises gilteritinib or a pharmaceutically acceptable salt thereof.

In one embodiment, the method comprises administering to the subject the therapeutically effective amount of the compound of Formula (I), (II), (III), (V), (VI), or (VII) (including a compound of Formula (Ia-5010-WO), (Ib-5010-WO), (Ic-5010-WO), (Id-5010-WO), (Ib-5010-WO50), (Id-5010-WO50), (IIa-5010-WO), (IIb-5010-WO), (11a-5010-WO50), (IIb-5010-WO50), (IIIa-5010-WO), (IIIb-5010-WO), (IIIa-5010-WO50), or (IIIb-5010-WO50)), or a salt, ester, solvate, optical isomer, geometric isomer, or salt of an isomer thereof or the a composition comprising a compound of Formula (I), (II), (III), (V), (VI), or (VII) (including a compound of Formula (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-50O-WO), (IIIb-5010-WO), (IIIa-501O-WO50), or (IIIb-5010-WO50)), or a salt, ester, solvate, optical isomer, geometric isomer, or salt of an isomer thereof about every 6 hours, every 12 hours, every 18 hours, once a day, every other day, every 3 days, every 4 days, every 5 days, every 6 days, or once a week. In one embodiment, the administration comprises parenteral administration, a mucosal administration, intravenous administration, subcutaneous administration, topical administration, intradermal administration, oral administration, sublingual administration, intranasal administration, or intramuscular administration. In one embodiment, the compound is administered to the subject in an amount of from about 0.005 mg/kg subject body weight to about 1,000 mg/kg subject body weight. In one embodiment, a lower dosage of the compound of Formula (I), (II), (III), (V), (VI), or (VII) (including a compound of Formula (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)), or a salt, ester, solvate, optical isomer, geometric isomer, or salt of an isomer thereof or a composition comprising a compound of Formula (I), (II), (III), (V), (VI), or (VII) (including a compound of Formula (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)) or a salt, ester, solvate, optical isomer, geometric isomer, or salt of an isomer thereof improves survivability in the subject compared to the dosage of the standard of care. In one embodiment, the method comprising administering to the subject a therapeutically effective amount of Compound 51 or a salt, ester, solvate, optical isomer, geometric isomer, or salt of an isomer thereof. In another embodiment, the method comprising administering to the subject a composition comprising a therapeutically effective amount of Compound 51 or a salt, ester, solvate, optical isomer, geometric isomer, or salt of an isomer thereof.

In one embodiment, the AML comprises AML with a splicing factor mutation, AML having enhanced IRAK4-Long expression and/or activity relative to IRAK4-Short, and/or AML which is not driven by FLT3 mutations but expresses IRAK4-Long.

In one embodiment, the method further comprises administering to the subject one or more additional therapies selected from: a chemotherapy agent, a BCL2 inhibitor, an immune modulator, a BTK inhibitor, a DNA methyltransferase inhibitor/hypomethylating agent, an anthracycline, a histone deacetylase (HDAC) inhibitor, a purine nucleoside analogue (antimetabolite), an isocitrate dehydrogenase 1 or 2 (IDH1 and/or IDH2) inhibitor, an antibody-drug conjugate, an mAbs/immunotherapy, a Plk inhibitor, a MEK inhibitor, a CDK inhibitor, a CDK9 inhibitor, a CDK8 inhibitor, a retinoic acid receptor agonist, a TP53 activator, a CELMoD, a smoothened receptor antagonist, an ERK inhibitor including an ERK2/MAPK1 or ERK1/MAPK3 inhibitor, a PI3K inhibitor, an mTOR inhibitor, a steroid or glucocorticoid, a steroid or glucocorticoid receptor modulator, an EZH2 inhibitor, a hedgehog (Hh) inhibitor, a Topoisomerase I inhibitor, a Topoisomerase II inhibitor, an aminopeptidase/Leukotriene A4 hydrolase inhibitor, a FLT3/Axl/ALK inhibitor, a FLT3/KIT/PDGFR, PKC, and/or KDR inhibitor, a Syk inhibitor, an E-selectin inhibitor, an NEDD8-activator, an MDM2 inhibitor, a PLK1 inhibitor, an Aura A inhibitor, an aurora kinase inhibitor, an EGFR inhibitor, an AuroraB/C/VEGFR1/2/3/FLT3/CSF-1R/Kit/PDGFRA/B inhibitor, an AKT 1, 2, and/or 3 inhibitor, a ABL1/2/SRC/EPHA2/LCK/YES1/KIT/PDGFRB/FYN inhibitor, a farnesyltransferase inhibitor, a BRAF/MAP2K1/MAP2K2 inhibitor, a Menin-KMT2A/MLL inhibitor, and a multikinase inhibitor.

In one embodiment, the AML is responsive to at least one of BCL2 inhibition, BTK inhibition, CDK inhibition, and DNA methyltransferase inhibition; or the AML is sensitive to anti-inflammatory glucocorticoids. In one embodiment, the additional therapy is at least one of a BCL2 inhibitor, a BTK inhibitor, a glucocorticoid, a CDK inhibitor, and a DNA methyltransferase inhibitor.

In one embodiment, the BCL2 inhibitor is venetoclax or a pharmaceutically acceptable salt thereof. In one embodiment, the AML is BCL2 inhibitor resistant. In one embodiment, the AML is venetoclax resistant. In one embodiment, the AML is BCL2 inhibitor resistant refractory AML. In one embodiment, the AML is venetoclax resistant refractory AML. In one embodiment, the AML is BCL2 inhibitor resistant relapsed AML. In one embodiment, the AML is venetoclax resistant relapsed AML.

In one embodiment, the BTK inhibitor is ibrutinib or a pharmaceutically acceptable salt thereof. In one embodiment, the AML is BTK inhibitor resistant. In one embodiment, the AML is ibrutinib resistant.

In one embodiment, the glucocorticoid is selected from dexamethasone, methylprednisolone, prednisolone, or a pharmaceutically acceptable salt of any one thereof. In one embodiment, the AML is sensitive to anti-inflammatory glucocorticoids. In one embodiment, the AML is dexamethasone, methylprednisolone, or prednisolone resistant.

In one embodiment, the CDK inhibitor is selected from CDK4/6 inhibitor palbociclib, CDK7 inhibitor THZ1, and/or CDK9 inhibitors BAY1251152 and atuveciclib, or a pharmaceutically acceptable salt of any one thereof. In one embodiment, the AML is CDK inhibitor resistant. In one embodiment, the AML is palbociclib, THZ1, BAY 12511152, or atuveciclib resistant.

In one embodiment, the DNA methyltransferase inhibitor is azacitidine or a pharmaceutically acceptable salt thereof. In one embodiment, the AML is DNA methyltransferase inhibitor resistant. In one embodiment, the AML is azacitidine resistant.

In one embodiment, the AML is BCL2 inhibitor and DNA methyltransferase inhibitor resistant. In one embodiment, the AML is venetoclax and azacitidine resistant. In one embodiment, the BCL2 inhibitor is venetoclax or a pharmaceutically acceptable salt thereof and the DNA methyltransferase inhibitor is azacitidine or a pharmaceutically acceptable salt thereof.

In one embodiment, the AML is FLT3 inhibitor resistant. In one embodiment, the AML is FLT3 inhibitor resistant refractory AMIL. In one embodiment, the AML is FLT3 inhibitor resistant relapsed AML.

In one embodiment, the compound of Formula (I), (II), (III), (V), (VI), or (VII) (including a compound of Formula (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)), or a salt, ester, solvate, optical isomer, geometric isomer, or salt of an isomer thereof or the composition comprising a compound of Formula (I), (II), (III), (V), (VI), or (VII) (including a compound of Formula (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)), or a salt, ester, solvate, optical isomer, geometric isomer, or salt of an isomer thereof and the one or more additional therapies are administered together in one administration or composition. In another embodiment, the compound of Formula (I), (II), (III), (V), (VI), or (VII) (including a compound of Formula (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)), or a salt, ester, solvate, optical isomer, geometric isomer, or salt of an isomer thereof or the composition comprising a compound of Formula (I), (II), (III), (V), (VI), or (VII) (including a compound of Formula (Ia-5010-WO), (Ib-5010-WO), (Ic-5010-WO), (Id-5010-WO), (Ib-5010-WO50), (Id-5010-WO50), (IIa-5010-WO), (IIb-5010-WO), (IIa-5010-WO50), (IIb-5010-WO50), (IIIa-5010-WO), (IIIb-5010-WO), (IIIa-5010-WO50), or (IIIb-5010-WO50)), or a salt, ester, solvate, optical isomer, geometric isomer, or salt of an isomer thereof and the one or more additional therapies are administered separately in more than one administration or more than one composition.

In one embodiment, the survivability is increased by inhibiting at least one of IRAK1, IRAK4, and FLT3 in the subject. In one embodiment, the survivability is increased by inhibiting at least two of IRAK1, IRAK4, and FLT3 in the subject. In one embodiment, the survivability is increased by inhibiting IRAK1 and IRAK4 in the subject. In one embodiment, the survivability is increased by inhibiting IRAK1, IRAK4, and FLT3 in the subject. In one embodiment, the FLT3 is selected from at least one of WT FLT3, activated FLT3, and mutated FLT3. In one embodiment, the mutated FLT3 is D835Y mutated FLT3 or F691L mutated FLT3.

In one embodiment, subject is a human. In one embodiment, the survivability of the human subject is increased by about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, or about 20 years compared to a human subject treated with a therapeutically effective amount of the standard of care for AML.

In another embodiment, the subject is a non-human mammal engrafted with AML cells. In one embodiment, the subject is a mouse engrafted with AML cells. In one embodiment, the AML cells are MOLM14-FLT3-ITD(D835Y) cells. In one embodiment, the survivability of the mouse subject is increased by about 1 day, about 2 days, about 5 days, about 10 days, about 15 days, about 20 days, about 25 days, about 30 days, about 35 days, about 40 days, about 45 days, about 50 days, about 55 days, about 60 days, about 65 days, about 70 days, about 75 days, about 80 days, about 85 days, or about 90 days compared to a mouse subject treated with a therapeutically effective amount of the standard of care for AML.

### Toxicity

The ratio between toxicity and therapeutic effect for a particular compound is its therapeutic index and can be expressed as the ratio between LD₅₀ (the amount of compound lethal in 50% of the population) and ED50 (the amount of compound effective in 50% of the population). Compounds that exhibit high therapeutic indices are preferred. Therapeutic index data obtained from in vitro assays, cell culture assays and/or animal studies can be used in formulating a range of dosages for use in humans. The dosage of such compounds preferably lies within a range of plasma concentrations that include the ED50 with little or no toxicity. The dosage can vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual practitioner in view of the patient's condition and the particular method in which the compound is used. For in vitro formulations, the exact formulation and dosage can be chosen by the individual practitioner in view of the patient's condition and the particular method in which the compound is used.

Having described the invention in detail, it will be apparent that modifications, variations, and equivalent embodiments are possible without departing from the scope of the invention defined in the appended claims. Furthermore, it should be appreciated that all examples in the present disclosure are provided as non-limiting examples.

The following clauses describe certain embodiments.

Clause 1. A compound of formula (I), (II), or (III):

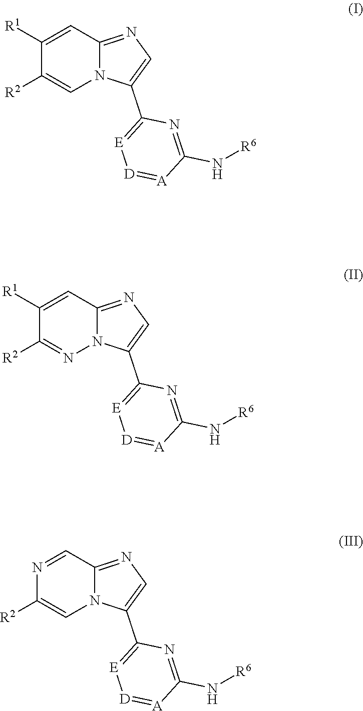

or a salt, ester, solvate, optical isomer, geometric isomer, salt of an isomer, prodrug, or derivative thereof,
wherein: A is selected from N and CR⁵; D is selected from N and CR⁴; E is selected from N and CR³; at least one of A, D, and E is N; R¹, R², R³, R⁴, and R⁵ are each independently selected from H, halogen, hydroxy, oxo, —CN, —C(═O)H, —C(═O)OH, C₁-C₇ alkyl, C₂-C₇ alkenyl, C₂-C₇ alkynyl, C₁-C₇ alkoxy, —C(═O)NR³¹R³², cycloalkyl, spiro-fused cycloalkyl, heterocyclyl, aryl, heteroaryl, or fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more of halogen, hydroxy, oxo, —C(═O)H, —C(═O)OH, nitro (—NO₂), —NH₂, —N(CH₃)₂, cyano (—CN), ethynyl (—CCH), propynyl, —SO₃H, heterocyclyl, aryl, heteroaryl, pyrrolyl, piperidyl, piperazinyl, morpholinyl, —C(═O)-morpholin-4-yl, —C(═O)NH₂, —C(═O)N(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl; R⁶ is

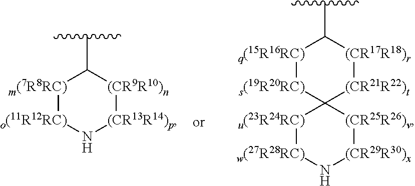

or C₃-C₆ cycloalkyl substituted with one or more —NR³³R³⁴; R⁷, R⁸, R⁹, R¹⁰, R¹¹, R¹², R¹³, and R¹⁴ are each independently selected from H, halogen, hydroxy, oxo, —CN, —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more halogen; R¹⁵, R¹⁶, R¹⁷, R¹⁸, R¹⁹, R²⁰, R²¹, R²², R²³, R²⁴, R²⁵, R²⁶, R²⁷, R²⁹, R²⁹, and R³⁰ are independently selected from H, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more halogen; R₃₁ and R₃₂ are each independently selected from H, C₁-C₆ alkyl, and C₃-C₆ cycloalkyl, wherein C₁-C₆ alkyl and C₃-C₆ cycloalkyl are optionally substituted with one or more halogen; R₃₃ and R³⁴ are each independently selected from H and C₁-C₆ alkyl; and m, n, o, p, q, r, s, t, u, v, w, and x are independently selected from 0, 1, 2, 3, 4, or 5, where q+r+s+t is at least 1, and where u+v+w+x is at least 1.

Clause 2. The compound of clause 1, wherein at least one of R⁷, R⁸, R⁹, R¹⁰, R¹¹, R¹², R¹³, and R¹⁴ is not H.

Clause 3. The compound of clause 1 or 2, wherein the compound of Formula (I), (II), or (III) is a compound of Formula (V), (VI), or (VII):

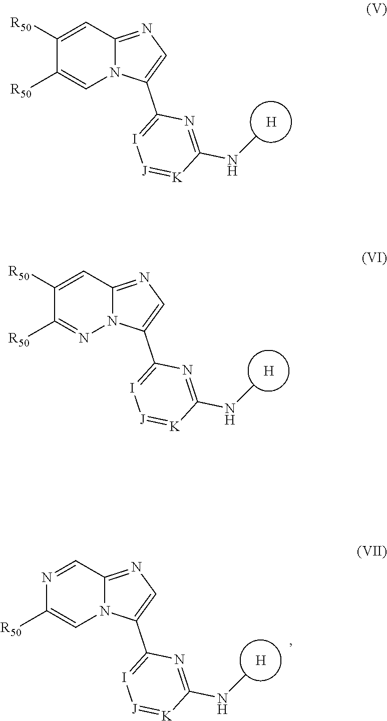

or a salt, ester, solvate, optical isomer, geometric isomer, or salt of an isomer thereof;
wherein: I is N or CR₅₁; J is N or CR₅₂; K is N or CR₅₃;

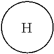

is selected from

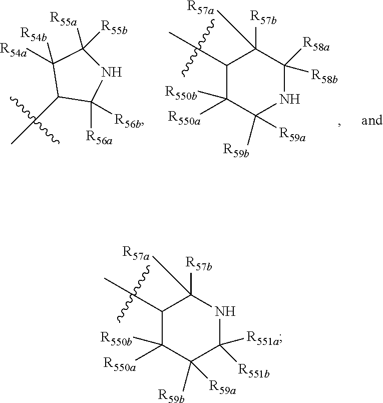

each R₅₀ is independently selected from H, halogen, C₁-C₆ alkyl, C₁-C₆ alkoxy, C₃-C₆ cycloalkyl, spiro-fused cycloalkyl, —O—(C₃-C₆ cycloalkyl), C₃-C₉ heteroaryl, C₂-C₉ heterocyclyl, and —C(═O)NR₅₅₂ₐR_{552b}, wherein C₁-C₆ alkyl and C₁-C₆ alkoxy are each optionally substituted with one or more substituents selected from —OH and halogen, and C₃-C₆ cycloalkyl, spiro-fused cycloalkyl, —O—(C₃-C₆ cycloalkyl), and C₂-C₉ heterocyclyl are each optionally substituted with one or more substituents selected from C₁-C₆ alkyl and halogen; R₅₁, R₅₂, and R₅₃ are each independently selected from H, C₁-C₆ alkoxy, and halogen; R₅₄ₐ, R_{54b}, R₅₅ₐ, R_{55b}, R₅₆ₐ, R_{56b}, R₅₇ₐ, R_{57b}, R₅₈ₐ, R_{58b}, R₅₉ₐ, R_{59b}, R₅₅₀ₐ, R_{550b}, R₅₅₁ₐ, and R_{551b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R₅₅₂ₐ and R_{552b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of I, J, or K is N.

Clause 4. The compound of clause 3, wherein one or more of R₅₄ₐ, R_{54b}, R₅₅ₐ, R_{55b}, R₅₆ₐ, R_{56b}, R₅₇ₐ, R_{57b}, R₅₈ₐ, R_{58b}, R₅₉ₐ, R_{59b}, R₅₅₀ₐ, R_{550b}, R₅₅₁ₐ, and R_{551b} is selected from halogen, —OH, optionally substituted C₁-C₆ alkyl, and optionally substituted C₁-C₆ alkoxy.

Clause 5. The compound of clause 1 or 2, wherein the compound of Formula (I) is a compound of Formula (Ib-5010-PR50):

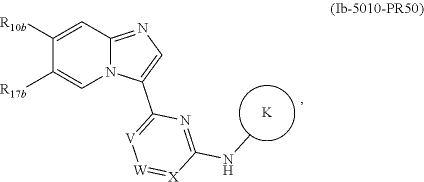

or a salt, ester, solvate, optical isomer, geometric isomer, or salt of an isomer thereof;
wherein: V is N or CR₁₁; W is N or CR₁₂; X is N or CR₁₃;

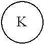

is

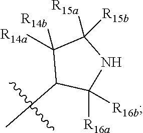

R_{10b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen; R_{17b} is selected from halogen, C₁-C₆ alkyl, C₁-C₆ alkoxy, C₃-C₆ cycloalkyl, —O—(C₃-C₆ cycloalkyl), C₃-C₉ heterocyclyl, imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen; R₁₁, R₁₂, and R₁₃ are each independently selected from H, C₁-C₆ alkoxy, and halogen; R₁₄ₐ, R_{14b}, R₁₅ₐ, R_{15b}, R₁₆ₐ, R_{16b}, R₁₈ₐ, and R_{18b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and one of V, W, or X is N.

Clause 6. The compound of clause 5, wherein at least one of (i)-(v) applies: (i) each of R_{14b}, R₁₅ₐ, R_{15b}, R₁₆ₐ, and R_{16b} is H and R₁₄ₐ is F; (ii) R₁₁, R₁₂, and R₁₃, if present, are H; (iii) V is CR₁₁ wherein Ru is F, W is CR₁₂ wherein R₁₂ is H, and X is N; (iv) R_{10b} is selected from H and —OCH₃; and (v) R_{17b} is selected from

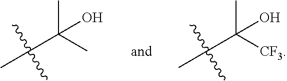

Clause 7. The compound of clause 5 or 6, wherein the compound of Formula (Ib-5010-PR50) is selected from:

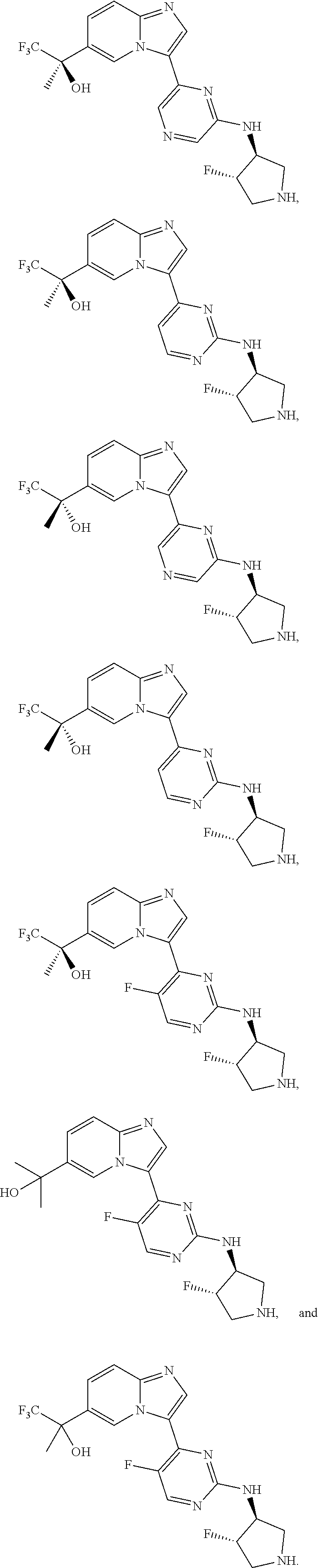

Clause 8. The compound of clause 1 or 2, wherein the compound of Formula (I) is a compound of Formula (Id-5010-PR50):

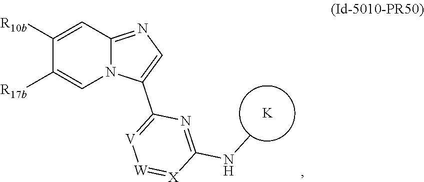

or a salt, ester, solvate, optical isomer, geometric isomer, or salt of an isomer thereof;
wherein: V is N or CR₁₁; W is N or CR₁₂; X is N or CR₁₃;

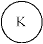

is

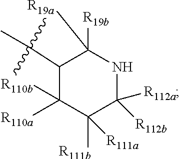

R_{10d} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen; R_{113d} is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and wherein C₃-C₆ cycloalkyl and —O—(C₃-C₆ cycloalkyl) are each optionally substituted with one or more substituents selected from C₁-C₆ alkyl and halogen; R₁₁, R₁₂, and R₁₃ are each independently selected from H, C₁-C₆ alkoxy, and halogen; R₁₈ₐ and R_{18b} are each independently selected from H, C₁-C₆ alkyl, and C₃-C₆ cycloalkyl, wherein C₁-C₆ alkyl and C₃-C₆ cycloalkyl are each optionally substituted with one or more halogen; R₁₉ₐ, R_{19b}, R₁₁₀ₐ, R_{110b}, R₁₁₁ₐ, R_{111b}, R₁₁₂ₐ, and R_{112b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and one of V, W, or X is N.

Clause 9. The compound of clause 8, wherein at least one of (i)-(vi) applies: (i) each of R₁₉ₐ, R_{19b}, R₁₁₀ₐ, R_{110b}, R₁₁₁ₐ, R_{111b}, R₁₁₂ₐ, and R_{112b} is H; (ii) each of R₁₉ₐ, R_{19b}, R_{110b}, R₁₁₁ₐ, R_{111b}, R₁₁₂ₐ, and R_{112b} is H and R₁₁₀ₐ is F; (iii) R₁₁, R₁₂, and R₁₃, if present, are H; (iv) V is CR₁₁ wherein R₁₁ is F, W is CR₁₂ wherein R₁₂ is H, and X is N; (v) R₁₀ₐ is selected from H and —OCH₃; and (vi) R_{113d} is selected from

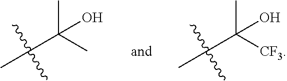

Clause 10. The compound of clause 8 or 9, wherein the compound of Formula (Id) is selected from:

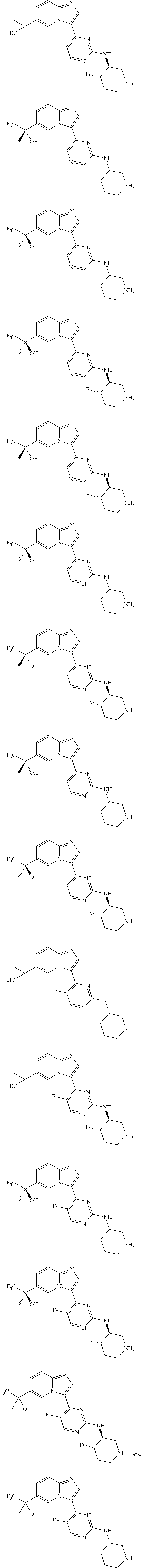

Clause 11. The compound of clause 1 or 2, wherein the compound of Formula (II) is a compound of Formula (IIa-5010-PR50):

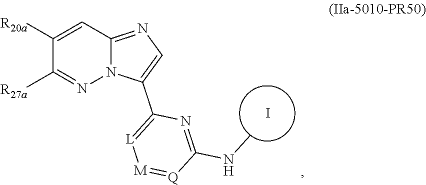

or a salt, ester, solvate, optical isomer, geometric isomer, or salt of an isomer thereof;
wherein: L is N or CR₂₁; M is N or CR₂₂; Q is N or CR₂₃;

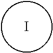

is

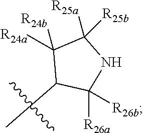

R₂₀ₐ is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen; R₂₇ₐ is selected from halogen, C₁-C₆ alkyl, C₁-C₆ alkoxy, C₃-C₆ cycloalkyl, spiro-fused cycloalkyl, —O—(C₃-C₆ cycloalkyl), C₂-C₆ heterocyclyl, imidazolyl, triazolyl, and —C(═O)NR₂₈ₐR_{28b}, wherein C₁-C₆ alkyl and C₁-C₆ alkoxy are each optionally substituted with one or more substituents selected from —OH and halogen, and C₃-C₆ cycloalkyl, spiro-cycloalkyl, —O—(C₃-C₆ cycloalkyl), and C₂-C₆ heterocyclyl are each optionally substituted with one or more substituents selected from C₁-C₆ alkyl and halogen; R₂₁, R₂₂, and R₂₃ are each independently selected from H, C₁-C₆ alkoxy, and halogen; R₂₄ₐ, R_{24b}, R₂₅ₐ, R_{25b}, R₂₆ₐ, and R_{26b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R₂₈ₐ and R_{28b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of L, M, or Q is N.

Clause 12. The compound of clause 11, wherein at least one of (i)-(v) applies: (i) each of R_{24b}, R₂₅ₐ, R_{25b}, R₂₆ₐ, and R_{26b} is H and R₂₄ₐ is F; (ii) R₂₁, R₂₂, and R₂₃, if present, are H; (iii) L is CR₂₁ wherein R₂₁ is selected from F, Cl, and —OCH₃, M is CR₂₂ wherein R₂₂ is H, and Q is N; (iv) R₂₀ₐ is selected from —OCH₃, unsubstituted —O—(C₃ cycloalkyl), and

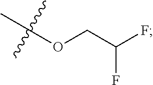 [SMILES: *OCC(F)F]

and
(v) R₂₇ₐ is selected from unsubstituted C₃-C₆ cycloalkyl,

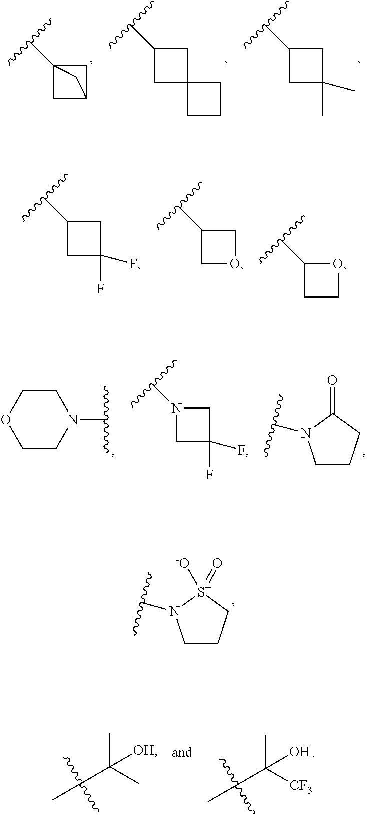

Clause 13. The compound of clause 11 or 12, wherein the compound of Formula (IIa-5010-PR50) is selected from:

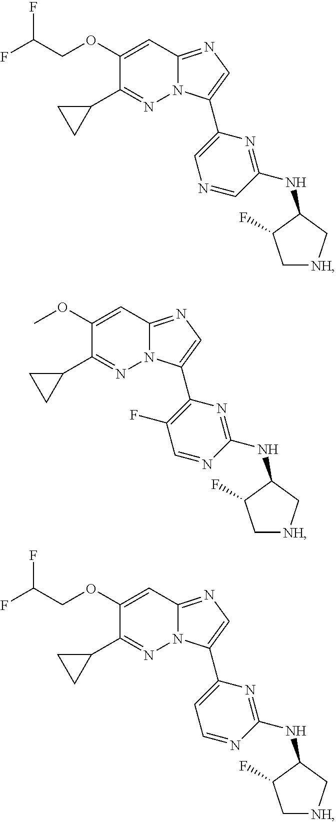 [SMILES: FC(F)COc1cc2ncc(-c3cncc(N[C@H]4CNC[C@@H]4F)n3)n2nc1C1CC1 ; COc1cc2ncc(-c3nc(N[C@H]4CNC[C@@H]4F)ncc3F)n2nc1C1CC1 ; FC(F)COc1cc2ncc(-c3ccnc(N[C@H]4CNC[C@@H]4F)n3)n2nc1C1CC1]

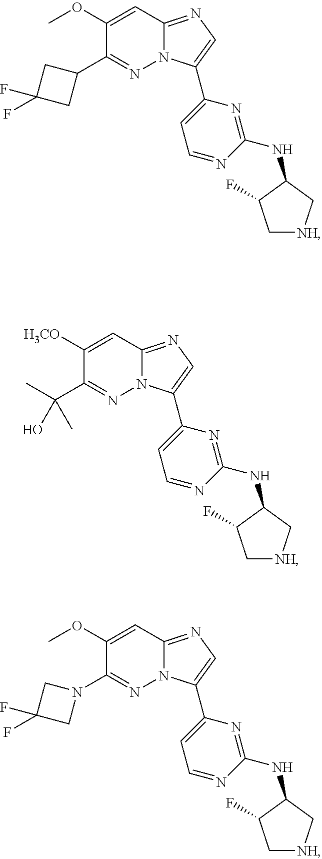 [SMILES: COc1cc2ncc(-c3ccnc(N[C@H]4CNC[C@@H]4F)n3)n2nc1C1CC(F)(F)C1 ; COc1cc2ncc(-c3ccnc(N[C@H]4CNC[C@@H]4F)n3)n2nc1C(C)(C)O ; COc1cc2ncc(-c3ccnc(N[C@H]4CNC[C@@H]4F)n3)n2nc1N1CC(F)(F)C1]

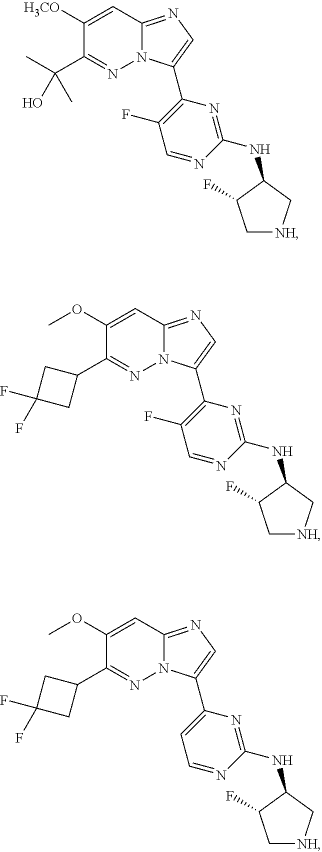 [SMILES: COc1cc2ncc(-c3nc(N[C@H]4CNC[C@@H]4F)ncc3F)n2nc1C(C)(C)O ; COc1cc2ncc(-c3nc(N[C@H]4CNC[C@@H]4F)ncc3F)n2nc1C1CC(F)(F)C1 ; COc1cc2ncc(-c3ccnc(N[C@H]4CNC[C@@H]4F)n3)n2nc1C1CC(F)(F)C1]

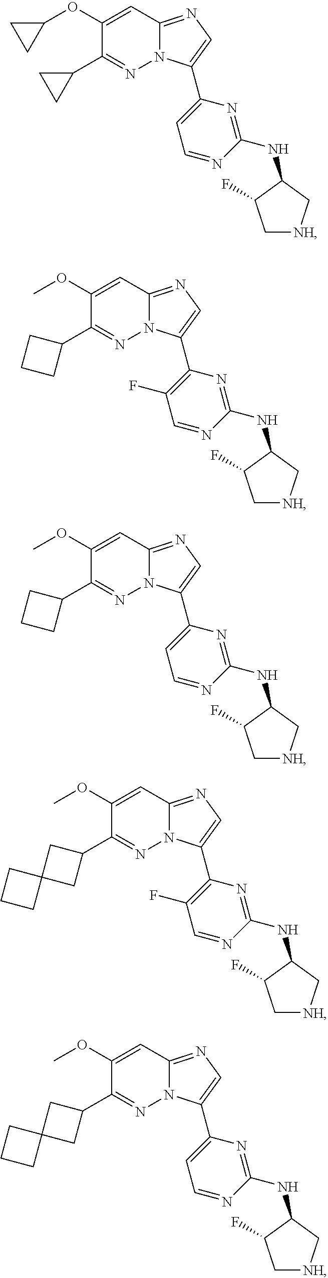 [SMILES: F[C@H]1CNC[C@@H]1Nc1nccc(-c2cnc3cc(OC4CC4)c(C4CC4)nn23)n1 ; COc1cc2ncc(-c3nc(N[C@H]4CNC[C@@H]4F)ncc3F)n2nc1C1CCC1 ; COc1cc2ncc(-c3ccnc(N[C@H]4CNC[C@@H]4F)n3)n2nc1C1CCC1 ; COc1cc2ncc(-c3nc(N[C@H]4CNC[C@@H]4F)ncc3F)n2nc1C1CC2(CCC2)C1 ; COc1cc2ncc(-c3ccnc(N[C@H]4CNC[C@@H]4F)n3)n2nc1C1CC2(CCC2)C1]

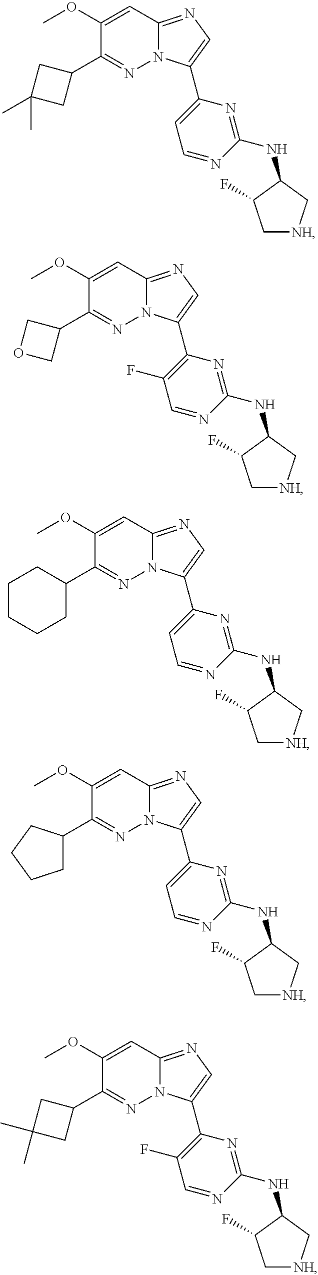 [SMILES: COc1cc2ncc(-c3ccnc(N[C@H]4CNC[C@@H]4F)n3)n2nc1C1CC(C)(C)C1 ; COc1cc2ncc(-c3nc(N[C@H]4CNC[C@@H]4F)ncc3F)n2nc1C1COC1 ; COc1cc2ncc(-c3ccnc(N[C@H]4CNC[C@@H]4F)n3)n2nc1C1CCCCC1 ; COc1cc2ncc(-c3ccnc(N[C@H]4CNC[C@@H]4F)n3)n2nc1C1CCCC1 ; COc1cc2ncc(-c3nc(N[C@H]4CNC[C@@H]4F)ncc3F)n2nc1C1CC(C)(C)C1]

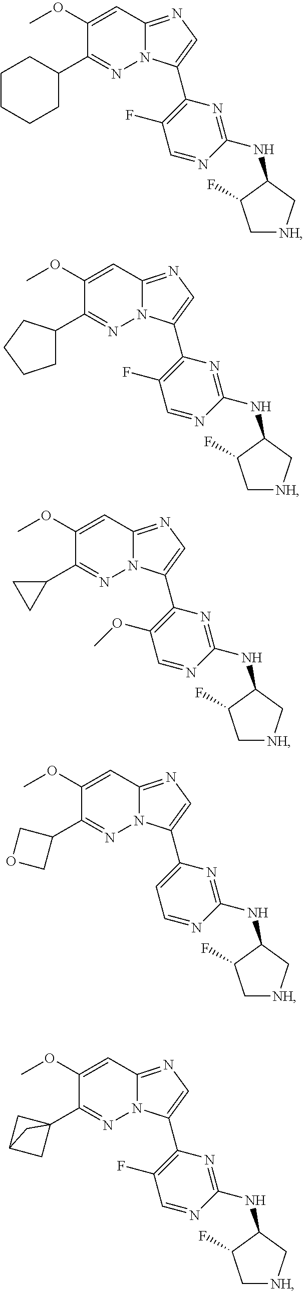 [SMILES: COc1cc2ncc(-c3nc(N[C@H]4CNC[C@@H]4F)ncc3F)n2nc1C1CCCCC1 ; COc1cc2ncc(-c3nc(N[C@H]4CNC[C@@H]4F)ncc3F)n2nc1C1CCCC1 ; COc1cnc(N[C@H]2CNC[C@@H]2F)nc1-c1cnc2cc(OC)c(C3CC3)nn12 ; COc1cc2ncc(-c3ccnc(N[C@H]4CNC[C@@H]4F)n3)n2nc1C1COC1 ; COc1cc2ncc(-c3nc(N[C@H]4CNC[C@@H]4F)ncc3F)n2nc1C12CC(C1)C2]

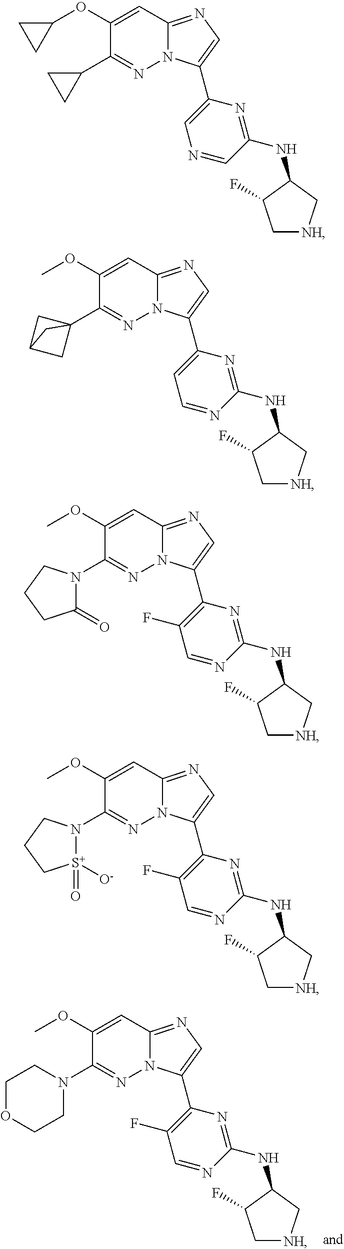

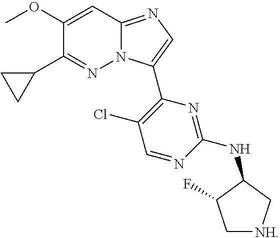 [SMILES: COc1cc2ncc(-c3nc(N[C@H]4CNC[C@@H]4F)ncc3Cl)n2nc1C1CC1]

Clause 14. The compound of clause 1 or 2, wherein the compound of Formula (II) is a compound of Formula (IIb-5010-WO50):

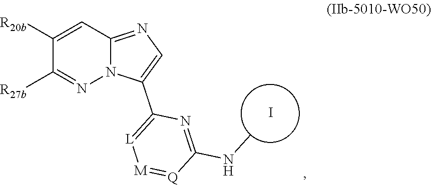

or a salt, ester, solvate, optical isomer, geometric isomer, or salt of an isomer thereof;
wherein: L is N or CR₂₁; M is N or CR₂₂; Q is N or CR₂₃;

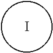

is

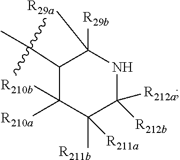

R_{20b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen; R_{27b} is selected from halogen, C₁-C₆ alkyl, C₁-C₆ alkoxy, C₃-C₆ cycloalkyl, spiro-cycloalkyl, —O—(C₃-C₆ cycloalkyl), C₂-C₆ heterocyclyl, imidazolyl, triazolyl, and —C(═O)NR₂₈ₐR_{28b}, wherein C₁-C₆ alkyl and C₁-C₆ alkoxy are each optionally substituted with one or more substituents selected from —OH and halogen, and C₃-C₆ cycloalkyl, spiro-cycloalkyl, —O—(C₃-C₆ cycloalkyl), and C₂-C₆ heterocyclyl are each optionally substituted with one or more substituents selected from C₁-C₆ alkyl and halogen; R₂₁, R₂₂, and R₂₃ are each independently selected from H, C₁-C₆ alkoxy, and halogen; R₂₉ₐ, R_{29b}, R₂₁₀ₐ, R_{210b}, R₂₁₁ₐ, R_{211b}, R₂₁₂ₐ, and R_{212b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R₂₈ₐ and R_{28b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of L, M, or Q is N.

Clause 15. The compound of clause 14, wherein at least one of (i)-(vi) applies: (i) each of R₂₉ₐ, R_{29b}, R₂₁₀ₐ, R_{211b}, R₂₁₁ₐ, R_{211b}, R₂₁₂ₐ, and R_{212b} is H; (ii) each of R₂₉ₐ, R_{29b}, R_{210b}, R₂₁₁ₐ, R_{211b}, R₂₁₂ₐ, and R_{212b} is H and R₂₁₀ₐ is F; (iii) R₂₁, R₂₂, and R₂₃, if present, are H; (iv) L is CR₂₁ wherein R₂₁ is selected from F, Cl, and —OCH₃, M is CR₂₂ wherein R₂₂ is H, and Q is N; (v) R_{20b} is selected from —OCH₃, unsubstituted —O—(C₃ cycloalkyl), and

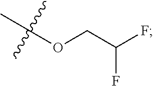 [SMILES: *OCC(F)F]

and (vi) R_{27b} is selected from unsubstituted C₃-C₆ cycloalkyl,

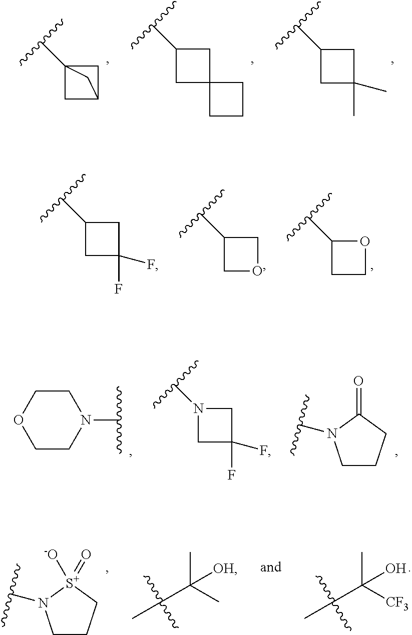

Clause 16. The compound of clause 14 or 15, wherein the compound of Formula (IIb) is selected from:

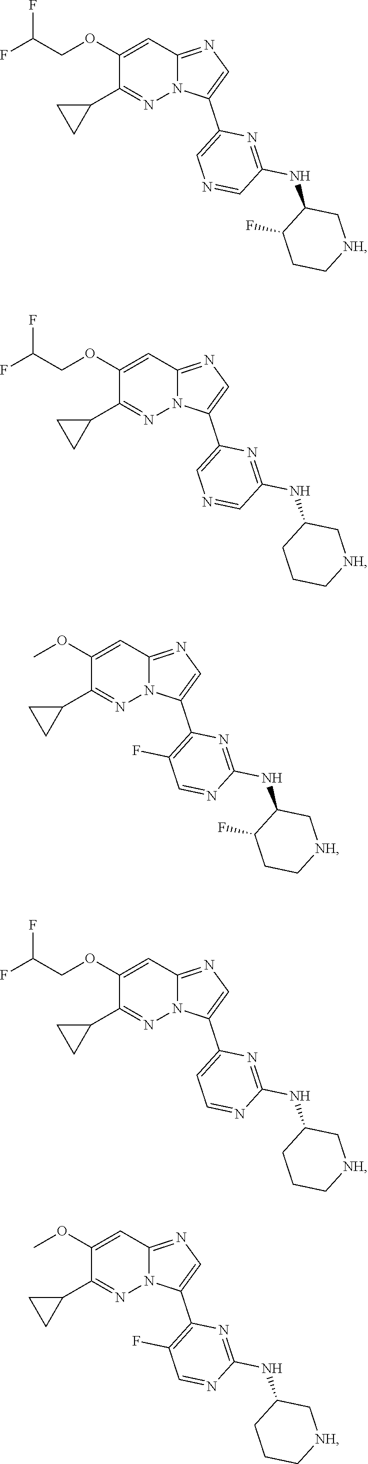 [SMILES: FC(F)COc1cc2ncc(-c3cncc(N[C@H]4CNCC[C@@H]4F)n3)n2nc1C1CC1 ; FC(F)COc1cc2ncc(-c3cncc(N[C@H]4CCCNC4)n3)n2nc1C1CC1 ; COc1cc2ncc(-c3nc(N[C@H]4CNCC[C@@H]4F)ncc3F)n2nc1C1CC1 ; FC(F)COc1cc2ncc(-c3ccnc(N[C@H]4CCCNC4)n3)n2nc1C1CC1 ; COc1cc2ncc(-c3nc(N[C@H]4CCCNC4)ncc3F)n2nc1C1CC1]

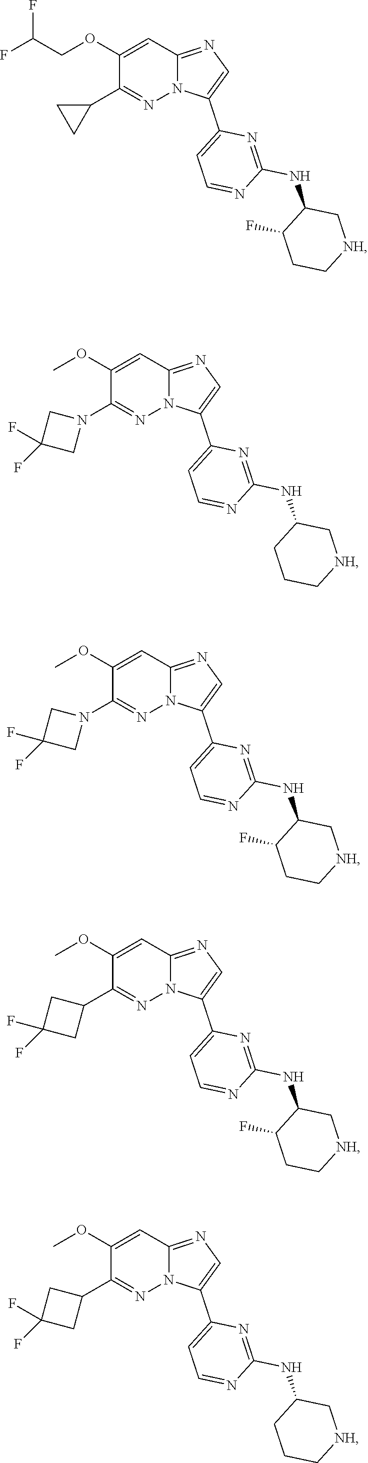 [SMILES: FC(F)COc1cc2ncc(-c3ccnc(N[C@H]4CNCC[C@@H]4F)n3)n2nc1C1CC1 ; COc1cc2ncc(-c3ccnc(N[C@H]4CCCNC4)n3)n2nc1N1CC(F)(F)C1 ; COc1cc2ncc(-c3ccnc(N[C@H]4CNCC[C@@H]4F)n3)n2nc1N1CC(F)(F)C1 ; COc1cc2ncc(-c3ccnc(N[C@H]4CNCC[C@@H]4F)n3)n2nc1C1CC(F)(F)C1 ; COc1cc2ncc(-c3ccnc(N[C@H]4CCCNC4)n3)n2nc1C1CC(F)(F)C1]

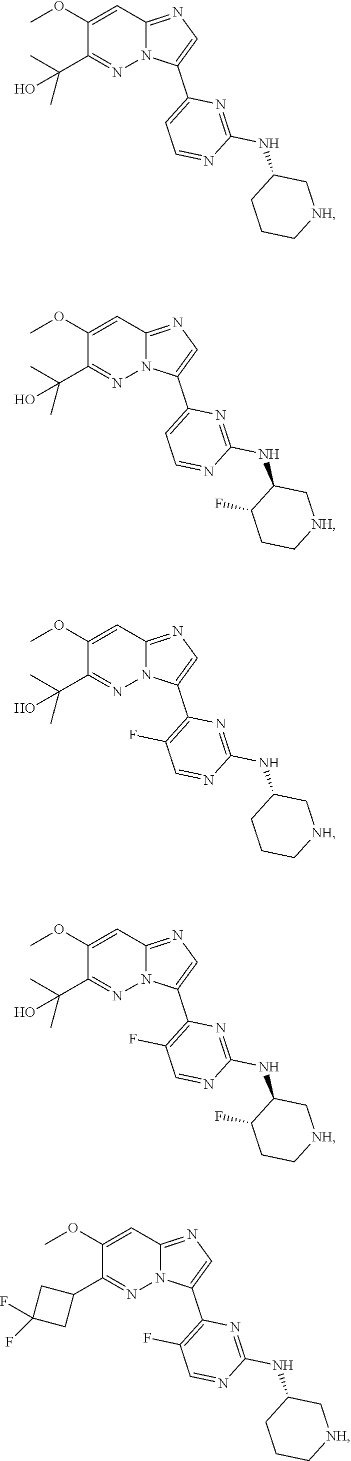 [SMILES: COc1cc2ncc(-c3ccnc(N[C@H]4CCCNC4)n3)n2nc1C(C)(C)O ; COc1cc2ncc(-c3ccnc(N[C@H]4CNCC[C@@H]4F)n3)n2nc1C(C)(C)O ; COc1cc2ncc(-c3nc(N[C@H]4CCCNC4)ncc3F)n2nc1C(C)(C)O ; COc1cc2ncc(-c3nc(N[C@H]4CNCC[C@@H]4F)ncc3F)n2nc1C(C)(C)O ; COc1cc2ncc(-c3nc(N[C@H]4CCCNC4)ncc3F)n2nc1C1CC(F)(F)C1]

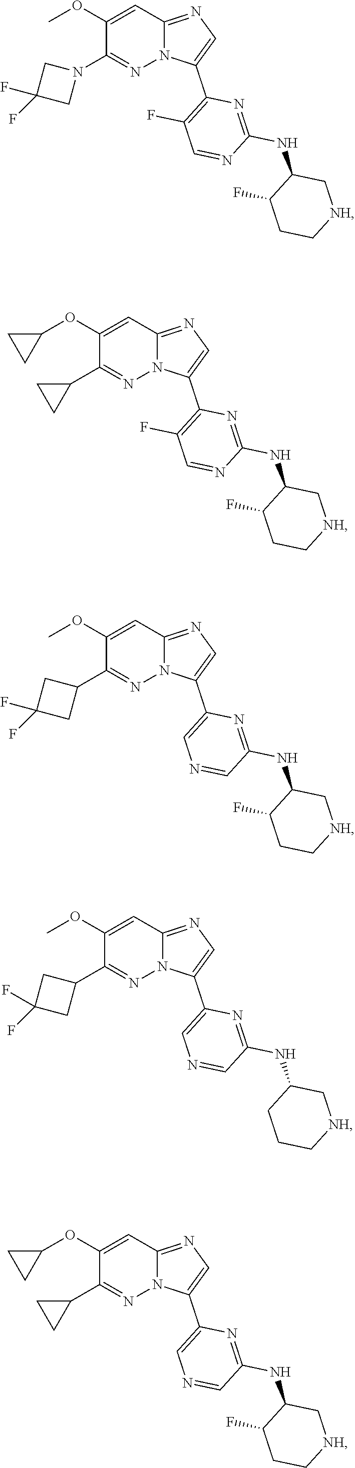 [SMILES: COc1cc2ncc(-c3nc(N[C@H]4CNCC[C@@H]4F)ncc3F)n2nc1N1CC(F)(F)C1 ; Fc1cnc(N[C@H]2CNCC[C@@H]2F)nc1-c1cnc2cc(OC3CC3)c(C3CC3)nn12 ; COc1cc2ncc(-c3cncc(N[C@H]4CNCC[C@@H]4F)n3)n2nc1C1CC(F)(F)C1 ; COc1cc2ncc(-c3cncc(N[C@H]4CCCNC4)n3)n2nc1C1CC(F)(F)C1 ; F[C@H]1CCNC[C@@H]1Nc1cncc(-c2cnc3cc(OC4CC4)c(C4CC4)nn23)n1]

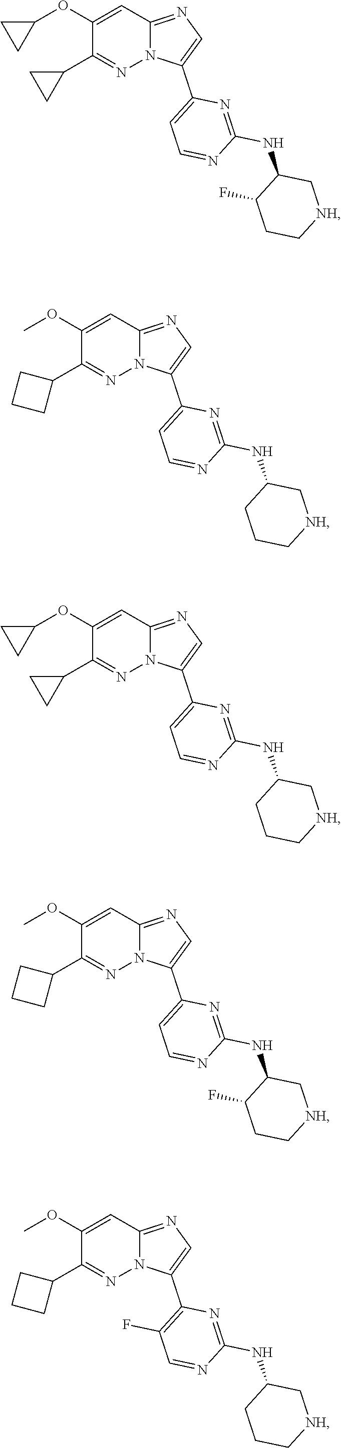 [SMILES: F[C@H]1CCNC[C@@H]1Nc1nccc(-c2cnc3cc(OC4CC4)c(C4CC4)nn23)n1 ; COc1cc2ncc(-c3ccnc(N[C@H]4CCCNC4)n3)n2nc1C1CCC1 ; c1cc(-c2cnc3cc(OC4CC4)c(C4CC4)nn23)nc(N[C@H]2CCCNC2)n1 ; COc1cc2ncc(-c3ccnc(N[C@H]4CNCC[C@@H]4F)n3)n2nc1C1CCC1 ; COc1cc2ncc(-c3nc(N[C@H]4CCCNC4)ncc3F)n2nc1C1CCC1]

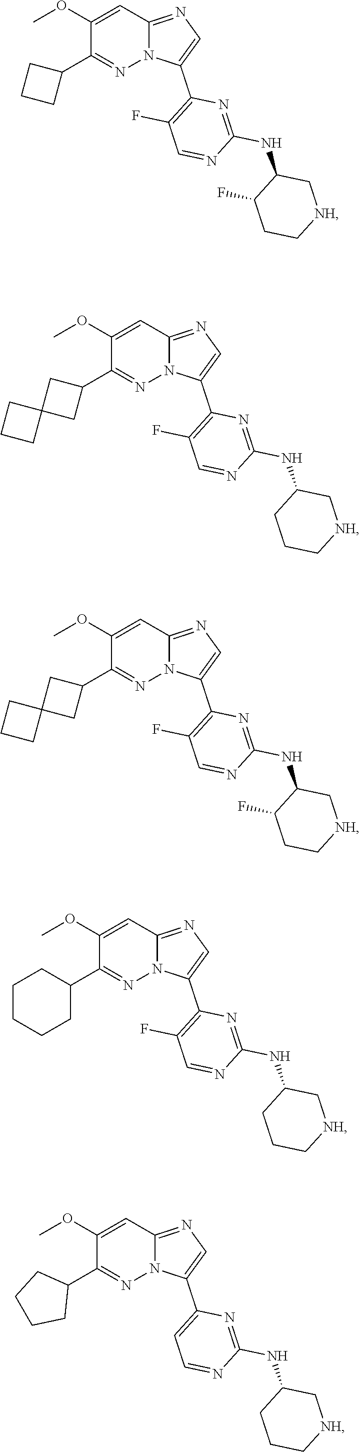 [SMILES: COc1cc2ncc(-c3nc(N[C@H]4CNCC[C@@H]4F)ncc3F)n2nc1C1CCC1 ; COc1cc2ncc(-c3nc(N[C@H]4CCCNC4)ncc3F)n2nc1C1CC2(CCC2)C1 ; COc1cc2ncc(-c3nc(N[C@H]4CNCC[C@@H]4F)ncc3F)n2nc1C1CC2(CCC2)C1 ; COc1cc2ncc(-c3nc(N[C@H]4CCCNC4)ncc3F)n2nc1C1CCCCC1 ; COc1cc2ncc(-c3ccnc(N[C@H]4CCCNC4)n3)n2nc1C1CCCC1]

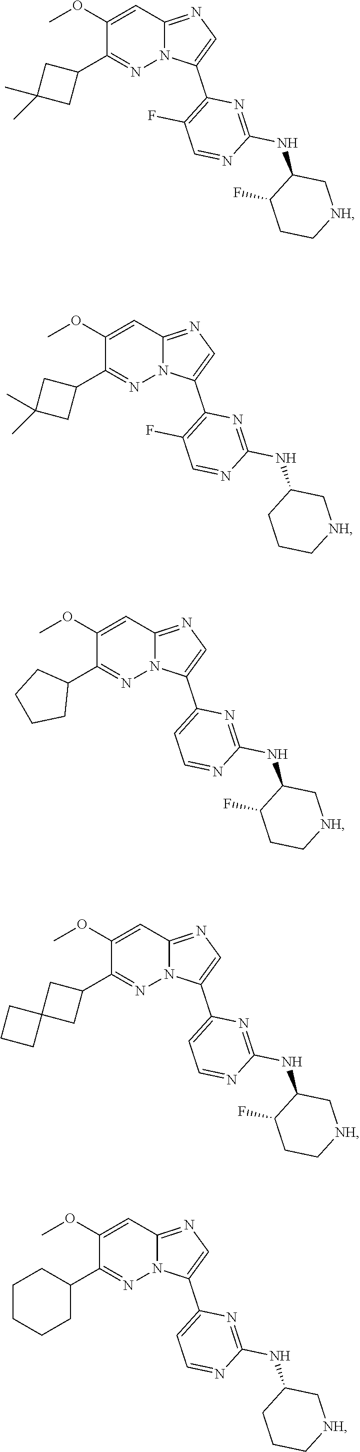 [SMILES: COc1cc2ncc(-c3nc(N[C@H]4CNCC[C@@H]4F)ncc3F)n2nc1C1CC(C)(C)C1 ; COc1cc2ncc(-c3nc(N[C@H]4CCCNC4)ncc3F)n2nc1C1CC(C)(C)C1 ; COc1cc2ncc(-c3ccnc(N[C@H]4CNCC[C@@H]4F)n3)n2nc1C1CCCC1 ; COc1cc2ncc(-c3ccnc(N[C@H]4CNCC[C@@H]4F)n3)n2nc1C1CC2(CCC2)C1 ; COc1cc2ncc(-c3ccnc(N[C@H]4CCCNC4)n3)n2nc1C1CCCCC1]

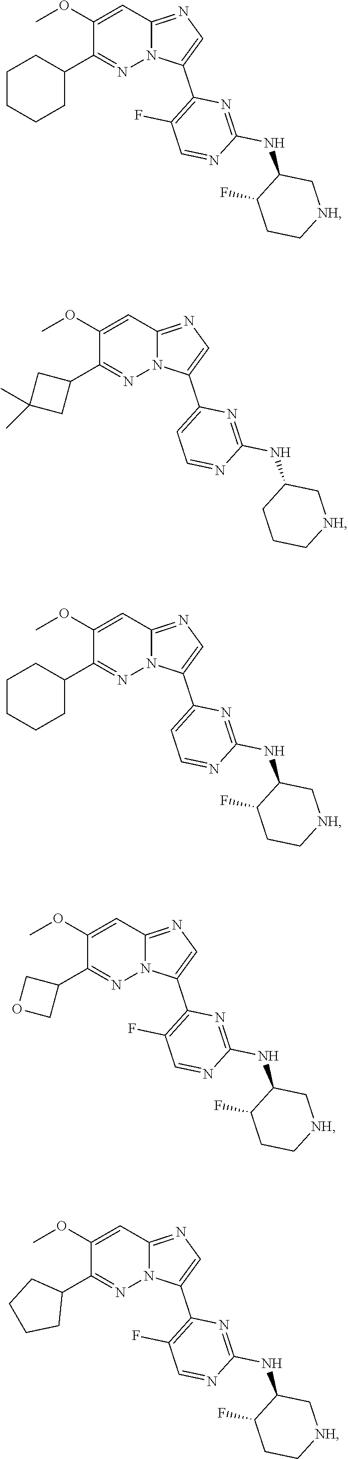 [SMILES: COc1cc2ncc(-c3nc(N[C@H]4CNCC[C@@H]4F)ncc3F)n2nc1C1CCCCC1 ; COc1cc2ncc(-c3ccnc(N[C@H]4CCCNC4)n3)n2nc1C1CC(C)(C)C1 ; COc1cc2ncc(-c3ccnc(N[C@H]4CNCC[C@@H]4F)n3)n2nc1C1CCCCC1 ; COc1cc2ncc(-c3nc(N[C@H]4CNCC[C@@H]4F)ncc3F)n2nc1C1COC1 ; COc1cc2ncc(-c3nc(N[C@H]4CNCC[C@@H]4F)ncc3F)n2nc1C1CCCC1]

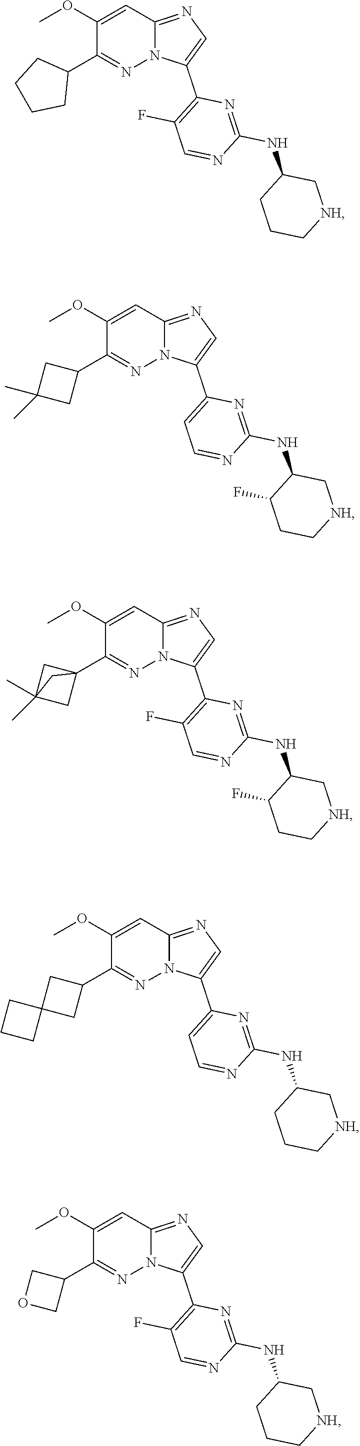

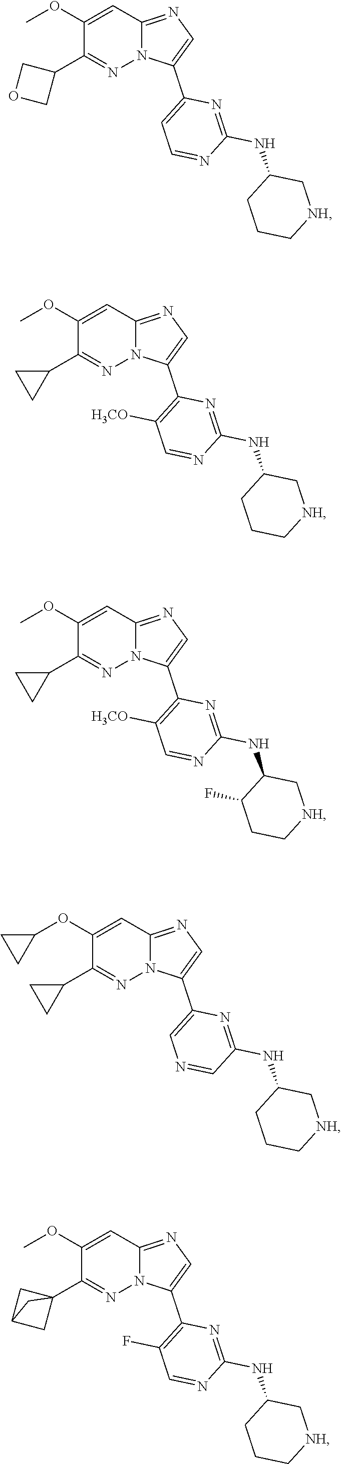 [SMILES: COc1cc2ncc(-c3ccnc(N[C@H]4CCCNC4)n3)n2nc1C1COC1 ; COc1cnc(N[C@H]2CCCNC2)nc1-c1cnc2cc(OC)c(C3CC3)nn12 ; COc1cnc(N[C@H]2CNCC[C@@H]2F)nc1-c1cnc2cc(OC)c(C3CC3)nn12 ; c1ncc(-c2cnc3cc(OC4CC4)c(C4CC4)nn23)nc1N[C@H]1CCCNC1 ; COc1cc2ncc(-c3nc(N[C@H]4CCCNC4)ncc3F)n2nc1C12CC(C1)C2]

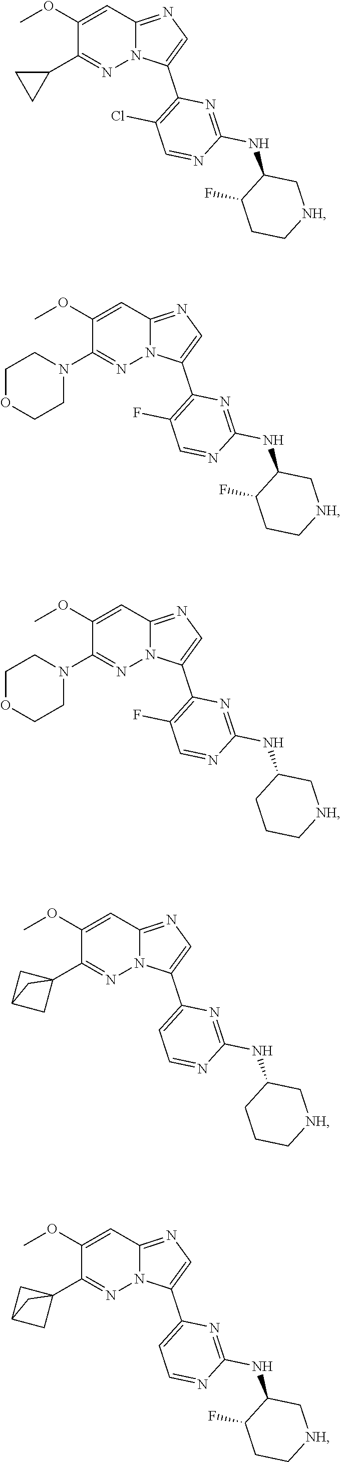 [SMILES: COc1cc2ncc(-c3nc(N[C@H]4CNCC[C@@H]4F)ncc3Cl)n2nc1C1CC1 ; COc1cc2ncc(-c3nc(N[C@H]4CNCC[C@@H]4F)ncc3F)n2nc1N1CCOCC1 ; COc1cc2ncc(-c3nc(N[C@H]4CCCNC4)ncc3F)n2nc1N1CCOCC1 ; COc1cc2ncc(-c3ccnc(N[C@H]4CCCNC4)n3)n2nc1C12CC(C1)C2 ; COc1cc2ncc(-c3ccnc(N[C@H]4CNCC[C@@H]4F)n3)n2nc1C12CC(C1)C2]

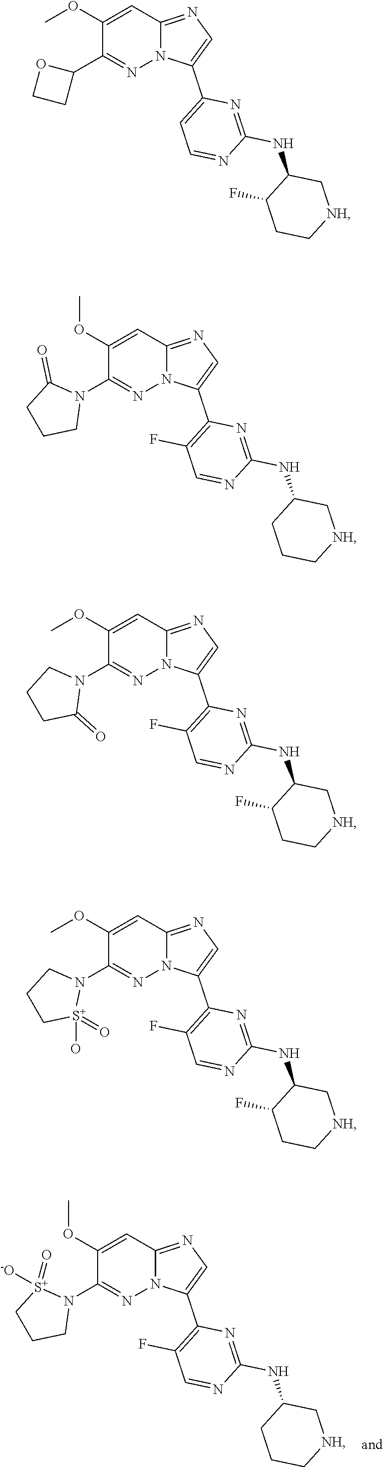

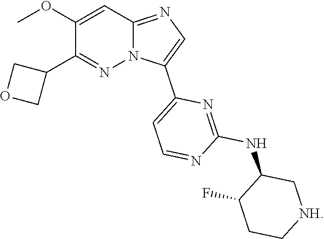 [SMILES: COc1cc2ncc(-c3ccnc(N[C@H]4CNCC[C@@H]4F)n3)n2nc1C1COC1]

Clause 17. The compound of clause 1 or 2, wherein the compound of Formula (III) is a compound of Formula (IIa-5010-PR50):

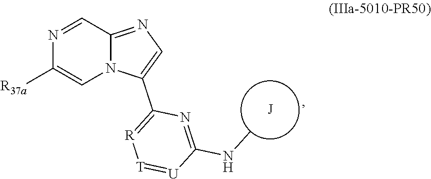

or a salt, ester, solvate, optical isomer, geometric isomer, or salt of an isomer thereof;
wherein: R is N or CR₃₁; T is N or CR₃₂; U is N or CR₃₃;

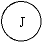

is

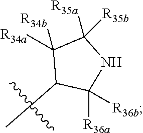

R₃₇ₐ is selected from halogen, C₁-C₆ alkyl, C₁-C₆ alkoxy, C₃-C₆ cycloalkyl, —O—(C₃-C₆ cycloalkyl), imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen; R₃₁, R₃₂, and R₃₃ are each independently selected from H, C₁-C₆ alkoxy, and halogen; R₃₄ₐ, R_{34b}, R₃₅ₐ, R_{35b}, R₃₆ₐ, and R^{36b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R₃₈ₐ and R_{38b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of R, T, or U is N.

Clause 18. The compound of clause 17, wherein at least one of (i)-(iv) applies: (i) each of R_{34b}, R₃₅ₐ, R_{35b}, R₃₆ₐ, and R_{36b} is H and R₃₄ₐ is F; (ii) R₃₁, R₃₂, and R₃₃, if present, are H; (iii) R is CR₃₁ wherein R₃₁ is F, T is CR₃₂ wherein R₃₂ is H, and U is N; and (iv) R₃₇ₐ is selected from

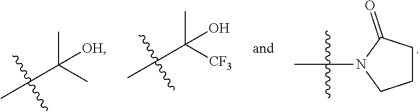

Clause 19. The compound of clause 17 or 18, wherein the compound of Formula IIIa is:

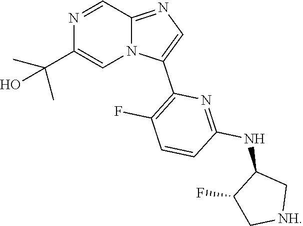 [SMILES: CC(C)(O)c1cn2c(-c3nc(N[C@H]4CNC[C@@H]4F)ccc3F)cnc2cn1]

Clause 20. The compound of clause 1 or 2, wherein the compound of Formula (III) is a compound of Formula (IIIb-5010-PR50):

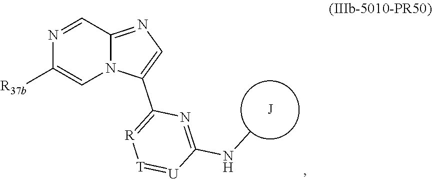

or a salt, ester, solvate, optical isomer, geometric isomer, or salt of an isomer thereof;
wherein: R is N or CR³; T is N or CR₃₂; U is N or CR₃₃;

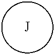

is

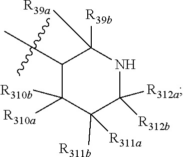

R^{37b} is selected from halogen, C₁-C₆ alkyl, C₁-C₆ alkoxy, C₃-C₆ cycloalkyl, —O—(C₃-C₆ cycloalkyl), imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen; R₃₁, R₃₂, and R₃₃ are each independently selected from H, C₁-C₆ alkoxy, and halogen; R₃₉ₐ, R_{39b}, R₃₁₀ₐ, R_{310b}, R₃₁₁ₐ, R_{311b}, R₃₁₂ₐ, and R_{312b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R₃₈ₐ and R_{38b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of R, T, or U is N.

Clause 21. The compound of clause 20, wherein at least one of (i)-(v) applies: (i) each of R₃₉ₐ, R_{39b}, R₃₁₀ₐ, R_{310b}, R₃₁₁ₐ, R_{311b}, R₃₁₂ₐ, and R_{312b} is H; (ii) each of R₃₉ₐ, R_{39b}, R_{310b}, R₃₁₁ₐ, R_{311b}, R₃₁₂ₐ, and R_{312b} is H and R₃₁₀ₐ is F; (iii) R₃₁, R₃₂, and R₃₃, if present, are H; (iv) R is CR₃₁ wherein R³¹ is F, T is CR₃₂ wherein R₃₂ is H, and U is N; and (v) R_{37b} is selected from

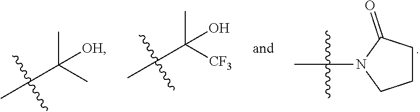

Clause 22. The compound of clause 20 or 21, wherein the compound of Formula (IIIb) is selected from:

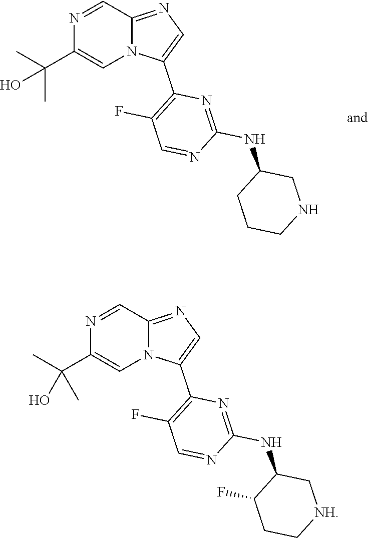 [SMILES: CC(C)(O)c1cn2c(-c3nc(N[C@@H]4CCCNC4)ncc3F)cnc2cn1 ; CC(C)(O)c1cn2c(-c3nc(N[C@H]4CNCC[C@@H]4F)ncc3F)cnc2cn1]

Clause 23. The compound of any one of clauses 1 to 22, wherein the compound is an inhibitor of at least one of IRAK1, IRAK4, and FLT3.

Clause 24. The compound of any one of clauses 1 to 23, wherein the compound is an inhibitor of at least two of IRAK1, IRAK4, and FLT3.

Clause 25a. The compound of any one of clauses 1 to 24, wherein the compound is an inhibitor of IRAK1 and IRAK4.

Clause 25b. The compound of any one of clauses 1 to 24, wherein the compound is an inhibitor of IRAK1 and IRAK4, and does not inhibit FLT3.

Clause 26. The compound of any one of clauses 1 to 24, wherein the compound is an inhibitor of IRAK1, IRAK4, and FLT3.

Clause 27. The compound of any one of clauses 23, 24, or 26, wherein FLT3 is selected from WT FLT3, activated FLT3, and mutated FLT3.

Clause 28. The compound of clause 27, wherein the mutated FLT3 is D835Y mutated FLT3 or F691L mutated FLT3

Clause 29. A composition comprising a compound of any one of clauses 1 to 28, wherein the composition further comprises a formulary ingredient, an adjuvant, or a carrier.

Clause 30. The composition of clause 29, wherein the composition is used in combination with one or more of: a chemotherapy agent, a BCL2 inhibitor, an immune modulator, a BTK inhibitor, a DNA methyltransferase inhibitor/hypomethylating agent, an anthracycline, a histone deacetylase (HDAC) inhibitor, a purine nucleoside analogue (antimetabolite), an isocitrate dehydrogenase 1 or 2 (IDH1 and/or IDH2) inhibitor, an antibody-drug conjugate, an mAbs/immunotherapy, a Plk inhibitor, a MEK inhibitor, a CDK inhibitor, a CDK9 inhibitor, a CDK8 inhibitor, a retinoic acid receptor agonist, a TP53 activator, a CELMoD, a smoothened receptor antagonist, an ERK inhibitor including an ERK2/MAPK1 or ERK1/MAPK3 inhibitor, a PI3K inhibitor, an mTOR inhibitor, a steroid or glucocorticoid, a steroid or glucocorticoid receptor modulator, an EZH2 inhibitor, a hedgehog (Hh) inhibitor, a Topoisomerase I inhibitor, a Topoisomerase II inhibitor, an aminopeptidase/Leukotriene A4 hydrolase inhibitor, a FLT3/Axl/ALK inhibitor, a FLT3/KIT/PDGFR, PKC, and/or KDR inhibitor, a Syk inhibitor, an E-selectin inhibitor, an NEDD8-activator, an MDM2 inhibitor, a PLK1 inhibitor, an Aura A inhibitor, an aurora kinase inhibitor, an EGFR inhibitor, an AuroraB/C/VEGFR1/2/3/FLT3/CSF-1R/Kit/PDGFRA/B inhibitor, an AKT 1, 2, and/or 3 inhibitor, a ABL1/2/SRC/EPHA2/LCK/YES1/KIT/PDGFRB/FYN inhibitor, a farnesyltransferase inhibitor, a BRAF/MAP2K1/MAP2K2 inhibitor, a Menin-KMT2A/MLL inhibitor, and a multikinase inhibitor.

Clause 31. The composition of clause 30, wherein the composition is used in combination with at least one of a BCL2 inhibitor, a BTK inhibitor, a glucocorticoid, a CDK inhibitor, and a DNA methyltransferase inhibitor.

Clause 32. The composition of clause 31, wherein the BCL2 inhibitor is venetoclax or a pharmaceutically acceptable salt thereof.

Clause 33. The composition of clause 31, wherein the BTK inhibitor is ibrutinib or a pharmaceutically acceptable salt thereof.

Clause 34. The composition of clause 31, wherein the glucocorticoid is selected from dexamethasone, methylprednisolone, prednisolone or a pharmaceutically acceptable salt of any one thereof.

Clause 35. The composition of clause 31, wherein the CDK inhibitor is a CDK4 inhibitor, a CDK6 inhibitor, a CDK7 inhibitor, and/or a CDK9 inhibitor.

Clause 36. The composition of clause 35, wherein the CDK inhibitor is selected from CDK4/6 inhibitor Palbociclib, CDK7 inhibitor THZ1, and/or CDK9 inhibitors BAY1251152 and Atuveciclib, or a pharmaceutically acceptable salt of any one thereof.

Clause 37. The composition of clause 31, wherein the DNA methyltransferase inhibitor is azacitidine or a pharmaceutically acceptable salt thereof.

Clause 38. A method of treating a disease or disorder in a subject, the method comprising administering to the subject a therapeutically effective amount of a compound of any one of clauses 1 to 28 or a composition of any one of clauses 29 to 37.

Clause 39. The method of clause 38, wherein the method comprises administering to the subject a composition comprising the therapeutically effective amount of the compound of clause 1 and a formulary ingredient, an adjuvant, or a carrier.

Clause 40. The method of clause 38 or 39, wherein the disease or disorder is responsive to at least one of interleukin-1 receptor-associated kinase (IRAK) inhibition and fins-like tyrosine kinase 3 (FLT3) inhibition.

Clause 41. The method of any one of clauses 38 to 40, wherein the administration comprises parenteral administration, a mucosal administration, intravenous administration, subcutaneous administration, topical administration, intradermal administration, oral administration, sublingual administration, intranasal administration, or intramuscular administration.

Clause 42. The method of any one of clauses 38 to 41, wherein the compound is administered to the subject in an amount of from about 0.005 mg/kg subject body weight to about 1,000 mg/kg subject body weight.

Clause 43. The method of any one of clauses 38 to 42, wherein the disease or disorder comprises a hematopoietic cancer.

Clause 44. The method of any one of clauses 38 to 42, wherein the disease or disorder comprises myelodysplastic syndrome (MDS) and/or acute myeloid leukemia (AML).

Clause 45. The method of any one of clauses 38 to 42, wherein the disease or disorder comprises lymphoma, leukemia, chronic lymphocytic leukemia (CLL), chronic myeloid leukemia (CML), acute lymphoblastic leukemia (ALL), bone marrow cancer, non-Hodgkin lymphoma, Waldenstrom's macroglobulinemia, B cell lymphoma, diffuse large B-cell lymphoma (DLBCL), DLBCL with MYD88 mutation, follicular lymphoma, or marginal zone lymphoma.

Clause 46. The method of any one of clauses 38 to 42, wherein the disease or disorder comprises at least one cancer selected from glioblastoma multiforme, endometrial cancer, melanoma, prostate cancer, lung cancer, breast cancer, kidney cancer, bladder cancer, basal cell carcinoma, thyroid cancer, squamous cell carcinoma, neuroblastoma, ovarian cancer, renal cell carcinoma, hepatocellular carcinoma, colon cancer, pancreatic cancer, rhabdomyosarcoma, meningioma, gastric cancer, Glioma, oral cancer, nasopharyngeal carcinoma, rectal cancer, stomach cancer, and uterine cancer, or combinations thereof.

Clause 47. The method of any one of clauses 38 to 42, wherein the disease or disorder comprises one or more inflammatory diseases or autoimmune disease selected from chronic inflammation, sepsis, rheumatoid arthritis, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, colitis, one or more inflammatory diseases or autoimmune disease characterized by overactive IRAK1 and/or IRAK4, or combinations thereof.

Clause 48. The method of any one of clauses 38 to 42, wherein the disease or disorder comprises: (i) MDS, MDS with a splicing factor mutation, MDS with a mutation in isocitrate dehydrogenase 1, MDS with a mutation in isocitrate dehydrogenase 2; or (ii) AML with a splicing factor mutation, AML having enhanced IRAK4-Long expression and/or activity relative to IRAK4-Short, and/or wherein the AML is not driven by FLT3 mutations but expresses IRAK4-Long.

Clause 49. The method of clause 48, wherein the MDS with a splicing factor mutation comprises MDS with a splicing factor mutation in U2AF1 or SF3B1 and the AML splicing factor mutation comprises AML with a splicing factor mutation in U2AF1 or SF3B1.

Clause 50. The method of any one of clauses 38 to 42, wherein the disease or disorder comprises DLBCL, and wherein the DLBCL comprises a L265P MYD88 mutant (ABC) subtype of DLBCL or a S219C MYD88 mutant (GCB) subtype of DLBCL.

Clause 51. The method of any one of clauses 38 to 50, further comprising administering to the subject one or more additional therapies selected from: a chemotherapy agent, a BCL2 inhibitor, an immune modulator, a BTK inhibitor, a DNA methyltransferase inhibitor/hypomethylating agent, an anthracycline, a histone deacetylase (HDAC) inhibitor, a purine nucleoside analogue (antimetabolite), an isocitrate dehydrogenase 1 or 2 (IDH1 and/or IDH2) inhibitor, an antibody-drug conjugate, an mAbs/immunotherapy, a Plk inhibitor, a MEK inhibitor, a CDK inhibitor, a CDK9 inhibitor, a CDK8 inhibitor, a retinoic acid receptor agonist, a TP53 activator, a CELMoD, a smoothened receptor antagonist, an ERK inhibitor including an ERK2/MAPK1 or ERK1/MAPK3 inhibitor, a PI3K inhibitor, an mTOR inhibitor, a steroid or glucocorticoid, a steroid or glucocorticoid receptor modulator, an EZH2 inhibitor, a hedgehog (Hh) inhibitor, a Topoisomerase I inhibitor, a Topoisomerase II inhibitor, an aminopeptidase/Leukotriene A4 hydrolase inhibitor, a FLT3/Axl/ALK inhibitor, a FLT3/KIT/PDGFR, PKC, and/or KDR inhibitor, a Syk inhibitor, an E-selectin inhibitor, an NEDD8-activator, an MDM2 inhibitor, a PLK1 inhibitor, an Aura A inhibitor, an aurora kinase inhibitor, an EGFR inhibitor, an AuroraB/C/VEGFR1/2/3/FLT3/CSF-1R/Kit/PDGFRA/B inhibitor, an AKT 1, 2, and/or 3 inhibitor, a ABL1/2/SRC/EPHA2/LCK/YES1/KIT/PDGFRB/FYN inhibitor, a farnesyltransferase inhibitor, a BRAF/MAP2K1/MAP2K2 inhibitor, a Menin-KMT2A/MLL inhibitor, and a multikinase inhibitor.

Clause 52. The method of any one of clauses 38 to 51, wherein the disease or disorder is responsive to at least one of BCL2 inhibition, BTK inhibition, CDK inhibition, and DNA methyltransferase inhibition; or wherein the disease or disorder is sensitive to anti-inflammatory glucocorticoids.

Clause 53. The method of clause 52, wherein the additional therapy is at least one of a BCL2 inhibitor, a BTK inhibitor, a glucocorticoid, a CDK inhibitor, and a DNA methyltransferase inhibitor.

Clause 54. The method of clause 53, wherein the BCL2 inhibitor is venetoclax or a pharmaceutically acceptable salt thereof.

Clause 55. The method of any one of clauses 38 to 54, wherein the disease or disorder is a BCL2 inhibitor resistant disease or disorder.

Clause 56. The method of any one of clauses 38 to 54, wherein the disease or disorder is a venetoclax resistant disease or disorder.

Clause 57. The method of any one of clauses 38 to 54, wherein the disease or disorder is BCL2 inhibitor resistant acute myeloid leukemia (AML).

Clause 58. The method of any one of clauses 38 to 54, wherein the disease or disorder is venetoclax resistant acute myeloid leukemia (AML).

Clause 59. The method of any one of clauses 38 to 54, wherein the disease or disorder is BCL2 inhibitor resistant refractory acute myeloid leukemia (AML).

Clause 60. The method of any one of clauses 38 to 54, wherein the disease or disorder is venetoclax resistant refractory acute myeloid leukemia (AML).

Clause 61. The method of any one of clauses 38 to 54, wherein the disease or disorder is BCL2 inhibitor resistant relapsed acute myeloid leukemia (AML).

Clause 62. The method of any one of clauses 38 to 54, wherein the disease or disorder is venetoclax resistant relapsed acute myeloid leukemia (AML).

Clause 63. The method of clause 53, wherein the BTK inhibitor is ibrutinib or a pharmaceutically acceptable salt thereof.

Clause 64. The method of any one of clauses 38 to 53, wherein the disease or disorder is a BTK inhibitor resistant disease or disorder.

Clause 65. The method of any one of clauses 38 to 53, wherein the disease or disorder is an ibrutinib resistant disease or disorder.

Clause 66. The method of clause 53, wherein the glucocorticoid is selected from dexamethasone, methylprednisolone, prednisolone, or a pharmaceutically acceptable salt of any one thereof.

Clause 67. The method of any one of clauses 38 to 53, wherein the disease or disorder is sensitive to anti-inflammatory glucocorticoids.

Clause 68. The method of any one of clauses 38 to 53, wherein the disease or disorder is a dexamethasone, methylprednisolone, or prednisolone resistant disease or disorder.

Clause 69. The method of clause 53, wherein the CDK inhibitor is selected from CDK4/6 inhibitor palbociclib, CDK7 inhibitor THZ1, and/or CDK9 inhibitors BAY1251152 and atuveciclib, or a pharmaceutically acceptable salt of any one thereof.

Clause 70. The method of any one of clauses 38 to 53, wherein the disease or disorder is a CDK inhibitor resistant disease or disorder.

Clause 71. The method of any one of clauses 38 to 53, wherein the disease or disorder is a palbociclib, THZ1, BAY 12511152, or atuveciclib resistant disease or disorder.

Clause 72. The method of clause 53, wherein the DNA methyltransferase inhibitor is azacitidine or a pharmaceutically acceptable salt thereof.

Clause 73. The method of any one of clauses 38 to 53, wherein the disease or disorder is a DNA methyltransferase inhibitor resistant disease or disorder.

Clause 74. The method of any one of clauses 38 to 53, wherein the disease or disorder is an azacitidine resistant disease or disorder.

Clause 75. The method of any one of clauses 38 to 53, wherein the disease or disorder is a BCL2 inhibitor and DNA methyltransferase inhibitor resistant disease or disorder.

Clause 76. The method of any one of clauses 38 to 53, wherein the disease or disorder is a venetoclax and azacitidine resistant disease or disorder.

Clause 77. The method of clause 53, wherein the BCL2 inhibitor is venetoclax or a pharmaceutically acceptable salt thereof and the DNA methyltransferase inhibitor is azacitidine or a pharmaceutically acceptable salt thereof.

Clause 78. The method of any one of clauses 38 to 53, wherein the disease or disorder is a FLT3 inhibitor resistant disease or disorder.

Clause 79. The method of any one of clauses 38 to 53, wherein the disease or disorder is FLT3 inhibitor resistant acute myeloid leukemia (AML).

Clause 80. The method of any one of clauses 38 to 53, wherein the disease or disorder is FLT3 inhibitor resistant refractory acute myeloid leukemia (AML).

Clause 81. The method of any one of clauses 38 to 53, wherein the disease or disorder is FLT3 inhibitor resistant relapsed acute myeloid leukemia (AML).

Clause 82. The method of clause 53, wherein the compound of any one of clauses 1 to 28 or the composition of any one of clauses 29 to 37 and the one or more additional therapies are administered together in one administration or composition.

Clause 83. The method of clause 53, wherein the compound of any one of clauses 1 to 28 or the composition of any one of clauses 29 to 37 and the one or more additional therapies are administered separately in more than one administration or more than one composition.

Clause 84. The method of any one of clauses 38 to 83, wherein the disease or disorder is alleviated by inhibiting at least one of IRAK1, IRAK4, and FLT3 in the subject

Clause 85. The method of any one of clauses 38 to 84, wherein the disease or disorder is alleviated by inhibiting at least two of IRAK1, IRAK4, and FLT3 in the subject.

Clause 86. The method of any one of clauses 38 to 85, wherein the disease or disorder is alleviated by inhibiting IRAK1 and IRAK4 in the subject.

Clause 87. The method of any one of clauses 38 to 85, wherein the disease or disorder is alleviated by inhibiting IRAK1, IRAK4, and FLT3 in the subject.

Clause 88. The method of any one of clauses 84, 85, or 87, wherein FLT3 is selected from WT FLT3, activated FLT3, and mutated FLT3.

Clause 89. The method of clause 88, wherein the mutated FLT3 is D835Y mutated FLT3 or F691L mutated FLT3.

Clause 90. The method of any one of clauses 38 to 83, wherein the compound or composition inhibits at least one of IRAK1, IRAK4, and FLT3 in the subject.

Clause 91. The method of any one of clauses 38 to 83, wherein the compound or composition inhibits at least two of IRAK1, IRAK4, and FLT3 in the subject.

Clause 92. The method of any one of clauses 38 to 83, wherein the compound or composition inhibits IRAK1 and IRAK4 in the subject.

Clause 101. A compound of formula (I), (II), or (III):

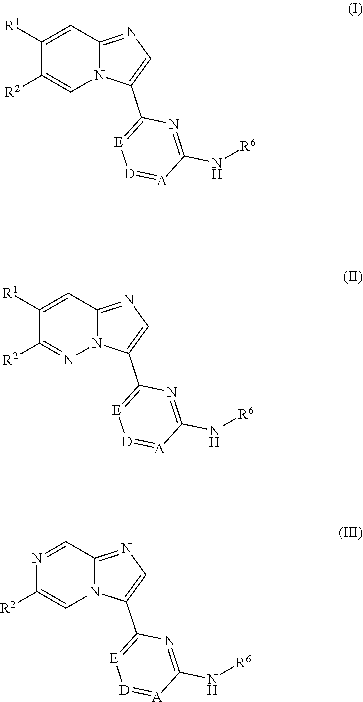

or a salt, ester, solvate, optical isomer, geometric isomer, salt of an isomer, prodrug, or derivative thereof,
wherein: A is selected from N and CR⁵; D is selected from N and CR⁴; E is selected from N and CR³; at least one of A, D, and E is N; R¹, R², R³, R⁴, and R⁵ are each independently selected from H, deuterium, halogen, hydroxy, oxo, —CN, —C(═O)H, —C(═O)OH, C₁-C₇ alkyl, C₂-C₇ alkenyl, C₂-C₇ alkynyl, C₁-C₇ alkoxy, —C(═O)NR³¹R³², cycloalkyl, —O-cycloalkyl, spiro-fused cycloalkyl, heterocyclyl, aryl, heteroaryl, or fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, —O-cycloalkyl, spiro-fused cycloalkyl, heterocyclyl, aryl, heteroaryl, or fused ring heteroaryl is optionally substituted with one or more of deuterium, halogen, hydroxy, oxo, —C(═O)H, —C(═O)OH, nitro (—NO₂), —NH₂, —N(CH₃)₂, cyano (—CN), ethynyl (—CCH), propynyl, —SO₃H, cycloalkyl, heterocyclyl, aryl, heteroaryl, pyrrolyl, piperidyl, piperazinyl, morpholinyl, —C(═O)-morpholin-4-yl, —C(═O)NH₂, —C(═O)N(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl; R⁶ is

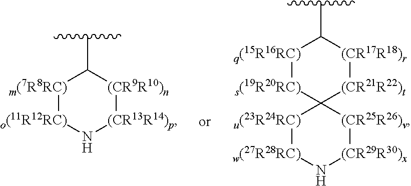

or C₃-C₆ cycloalkyl substituted with one or more —NR¹³R³⁴; R⁷, R⁸, R⁹, R¹⁰, R¹¹, R¹², R¹³, and R¹⁴ are each independently selected from H, deuterium, halogen, hydroxy, oxo, —CN, —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more halogen; R¹⁵, R¹⁶, R¹⁷, R¹⁸, R¹⁹, R²⁰, R²¹, R²², R²³, R²⁴, R²⁵, R²⁶, R²⁷, R²⁹, R²⁹, and R³⁰ are independently selected from H, deuterium, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more halogen; R₃₁ and R₃₂ are each independently selected from H, C₁-C₆ alkyl, and C₃-C₆ cycloalkyl, wherein C₁-C₆ alkyl and C₃-C₆ cycloalkyl are optionally substituted with one or more halogen; R₃₃ and R³⁴ are each independently selected from H and C₁-C₆ alkyl; and m, n, o, p, q, r, s, t, u, v, w, and x are independently selected from 0, 1, 2, 3, 4, or 5, where q+r+s+t is at least 1, and where u+v+w+x is at least 1.

Clause 102. The compound of clause 101, wherein at least one of R⁷, R⁸, R⁹, R¹⁰, R¹¹, R¹², R¹³, and R¹⁴ is not H.

Clause 103. The compound of clause 101 or 102, wherein the compound of Formula (I), (II), or (III) is a compound of Formula (V), (VI), or (VII):

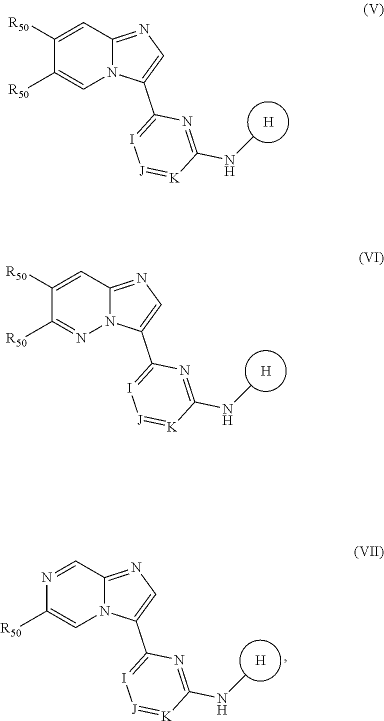

or a salt, ester, solvate, optical isomer, geometric isomer, or salt of an isomer thereof;
wherein: I is N or CR₅₁; J is N or CR₅₂; K is N or CR₅₃;

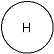

is selected from

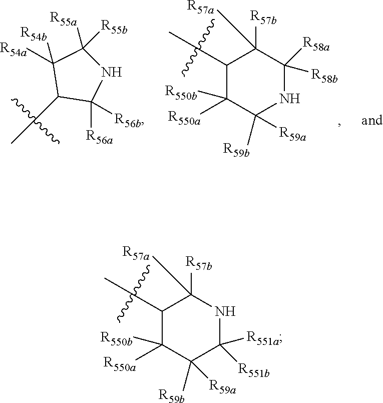

each R₅₀ is independently selected from H, halogen, C₁-C₆ alkyl, C₁-C₆ alkoxy, C₃-C₆ cycloalkyl, spiro-fused cycloalkyl, —O—(C₃-C₆ cycloalkyl), C₃-C₉ heteroaryl, C₂-C₉ heterocyclyl, and —C(═O)NR₅₅₂ₐR_{552b}, wherein C₁-C₆ alkyl and C₁-C₆ alkoxy are each optionally substituted with one or more substituents selected from —OH, deuterium, and halogen, and wherein C₃-C₆ cycloalkyl, spiro-fused cycloalkyl, —O—(C₃-C₆ cycloalkyl), and C₂-C₉ heterocyclyl are each optionally substituted with one or more substituents selected from —OH, C₁-C₆ alkyl, and halogen; R₅₁, R₅₂, and R₅₃ are each independently selected from H, C₁-C₆ alkoxy, and halogen; R₅₄ₐ, R_{54b}, R₅₅ₐ, R_{55b}, R₅₆ₐ, R_{56b}, R₅₇ₐ, R_{57b}, R₅₈ₐ, R_{58b}, R₅₉ₐ, R_{59b}, R₅₅₀ₐ, R_{550b}, R₅₅₁ₐ, and R_{551b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R₅₅₂ₐ and R_{552b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of I, J, or K is N.

Clause 104. The compound of clause 103, wherein one or more of R₅₄ₐ, R_{54b}, R₅₅ₐ, R_{55b}, R₅₆ₐ, R_{56b}, R₅₇ₐ, R_{57b}, R₅₅ₐ, R_{58b}, R₅₉ₐ, R_{59b}, R₅₅₀ₐ, R_{550b}, R₅₅₁ₐ, and R_{551b} is selected from halogen, —OH, optionally substituted C₁-C₆ alkyl, and optionally substituted C₁-C₆ alkoxy.

Clause 105. The compound of clause 101 or 102, wherein the compound of Formula (I) is a compound of Formula (Ib-5010-WO50):

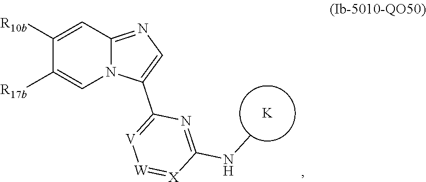

or a salt, ester, solvate, optical isomer, geometric isomer, or salt of an isomer thereof;
wherein: V is N or CR₁₁; W is N or CR₁₂; X is N or CR₁₃;

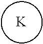

is

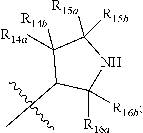

R_{10b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from C₁-C₆ alkyl and halogen; R_{17b} is selected from halogen, C₁-C₆ alkyl, C₁-C₆ alkoxy, C₃-C₆ cycloalkyl, —O—(C₃-C₆ cycloalkyl), C₃-C₉ heterocyclyl, imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from —OH, C₁-C₆ alkyl, and halogen; R₁₁, R₁₂, and R₁₃ are each independently selected from H, C₁-C₆ alkoxy, and halogen; R₁₄ₐ, R_{14b}, R₁₅ₐ, R_{15b}, R₁₆ₐ, R_{16b}, R₁₈ₐ, and R_{18b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and one of V, W, or X is N.

Clause 106. The compound of clause 105, wherein at least one of (i)-(v) applies: (i) each of R_{14b}, R₁₅ₐ, R_{15b}, R₁₆ₐ, and R_{16b} is H and R₁₄ₐ is F; (ii) R₁₁, R₁₂, and R₁₃, if present, are H; (iii) V is CR₁₁ wherein R₁₁ is F, W is CR₁₂ wherein R₁₂ is H, and X is N; (iv) R_{10b} is selected from H and —OCH₃; and (v) R_{17b} is selected from

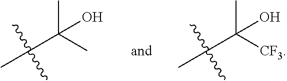

Clause 107. The compound of clause 105 or 106, with the provisos that: when R_{17b} is

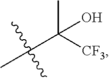 [SMILES: *C(C)(O)C(F)(F)F]

R_{10b} is H; and when R_{17b} is

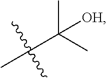 [SMILES: *C(C)(C)O]

at least one of R₁₁, R₁₂, and R₁₃ is present and selected from C₁-C₆ alkoxy and halogen.

Clause 108. The compound of any one of clauses 105 to 107, wherein the compound of Formula (Ib-5010-WO50) is selected from:

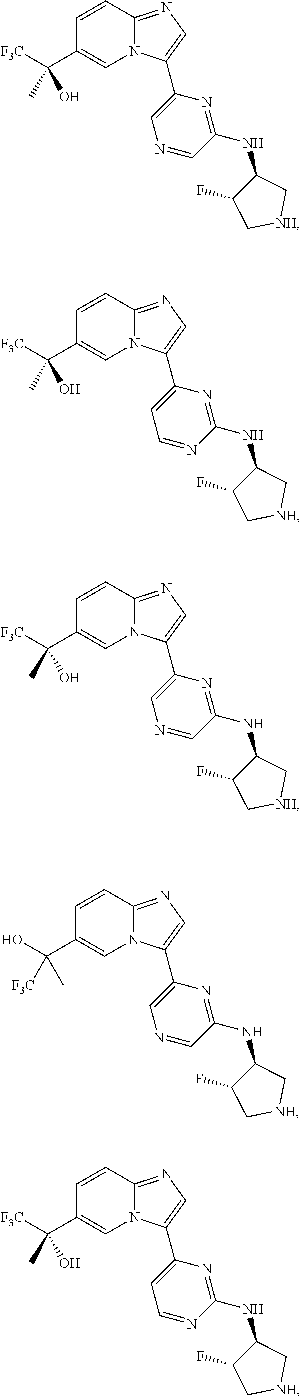 [SMILES: C[C@@](O)(c1ccc2ncc(-c3cncc(N[C@H]4CNC[C@@H]4F)n3)n2c1)C(F)(F)F ; C[C@@](O)(c1ccc2ncc(-c3ccnc(N[C@H]4CNC[C@@H]4F)n3)n2c1)C(F)(F)F ; C[C@](O)(c1ccc2ncc(-c3cncc(N[C@H]4CNC[C@@H]4F)n3)n2c1)C(F)(F)F ; CC(O)(c1ccc2ncc(-c3cncc(N[C@H]4CNC[C@@H]4F)n3)n2c1)C(F)(F)F ; C[C@](O)(c1ccc2ncc(-c3ccnc(N[C@H]4CNC[C@@H]4F)n3)n2c1)C(F)(F)F]

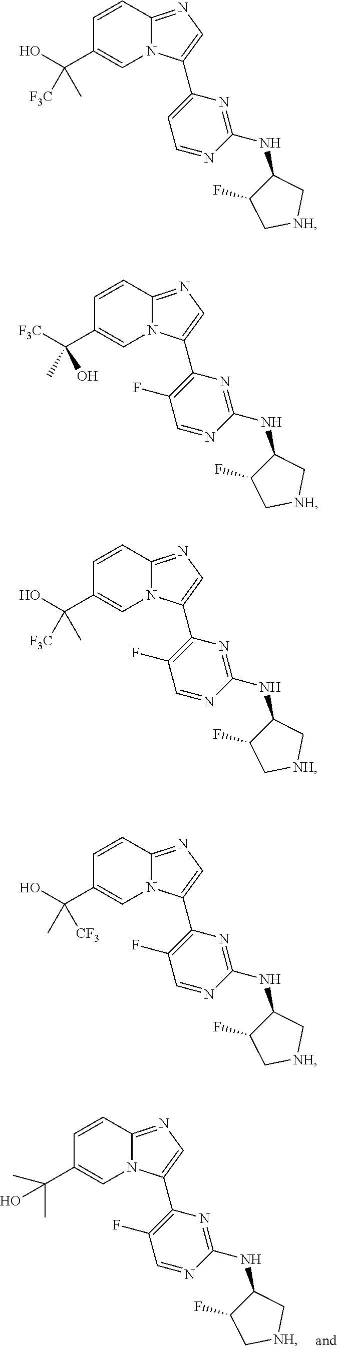 [SMILES: CC(O)(c1ccc2ncc(-c3ccnc(N[C@H]4CNC[C@@H]4F)n3)n2c1)C(F)(F)F ; C[C@@](O)(c1ccc2ncc(-c3nc(N[C@H]4CNC[C@@H]4F)ncc3F)n2c1)C(F)(F)F ; CC(O)(c1ccc2ncc(-c3nc(N[C@H]4CNC[C@@H]4F)ncc3F)n2c1)C(F)(F)F ; CC(O)(c1ccc2ncc(-c3nc(N[C@H]4CNC[C@@H]4F)ncc3F)n2c1)C(F)(F)F ; CC(C)(O)c1ccc2ncc(-c3nc(N[C@H]4CNC[C@@H]4F)ncc3F)n2c1]

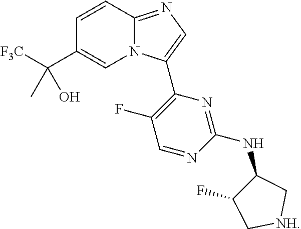 [SMILES: CC(O)(c1ccc2ncc(-c3nc(N[C@H]4CNC[C@@H]4F)ncc3F)n2c1)C(F)(F)F]

Clause 109. The compound of clause 101 or 102, wherein the compound of Formula (I) is a compound of Formula (Id-5010-WO50):

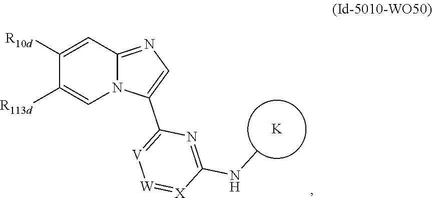

or a salt, ester, solvate, optical isomer, geometric isomer, or salt of an isomer thereof;
wherein: V is N or CR₁₁; W is N or CR₁₂; X is N or CR₁₃;

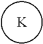

is

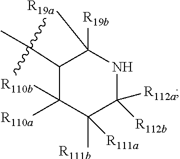

R_{10d} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and halogen, and C₃-C₆ cycloalkyl and wherein —O—(C₃-C₆ cycloalkyl) are each optionally substituted with one or more substituents selected from C₁-C₆ alkyl and halogen; R_{113d} is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from —OH, C₁-C₆ alkyl, and halogen; R₁₁, R₁₂, and R₁₃ are each independently selected from H, C₁-C₆ alkoxy, and halogen; R₁₈ₐ and R_{18b} are each independently selected from H, C₁-C₆ alkyl, and C₃-C₆ cycloalkyl, wherein C₁-C₆ alkyl and C₃-C₆ cycloalkyl are each optionally substituted with one or more halogen; R₁₉ₐ, R_{19b}, R₁₁₀ₐ, R_{110b}, R₁₁₁ₐ, R_{111b}, R₁₁₂ₐ, and R_{112b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and one of V, W, or X is N.

Clause 110. The compound of clause 109, wherein at least one of (i)-(vi) applies: (i) each of R₁₉ₐ, R_{19b}, R₁₁₀ₐ, R_{110b}, R₁₁₁ₐ, R_{111b}, R₁₁₂ₐ, and R_{112b} is H; (ii) each of R₁₉ₐ, R_{19b}, R_{110b}, R₁₁₁ₐ, R_{111b}, R₁₁₂ₐ, and R_{112b} is H and R₁₁₀ₐ is F; (iii) R₁₁, R₁₂, and R₁₃, if present, are H; (iv) V is CR₁₁ wherein R₁ is F, W is CR₁₂ wherein R₁₂ is H, and X is N; (v) R₁₀ₐ is selected from H and —OCH₃; and (vi) R_{113d} is selected from

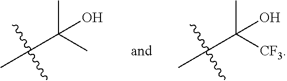

Clause 111. The compound of clause 109 or 110, with the provisos that: when R_{113d}

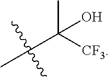 [SMILES: *C(C)(O)C(F)(F)F]

R_{10d} is H; and when R_{113d} is

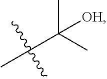 [SMILES: *C(C)(C)O]

at least one of R₁₁, R₁₂, and R₁₃ is present and selected from C₁-C₆ alkoxy and halogen.

Clause 112. The compound of any one of clauses 109 to 111, wherein the compound of Formula (Id-5010-WO50) is selected from:

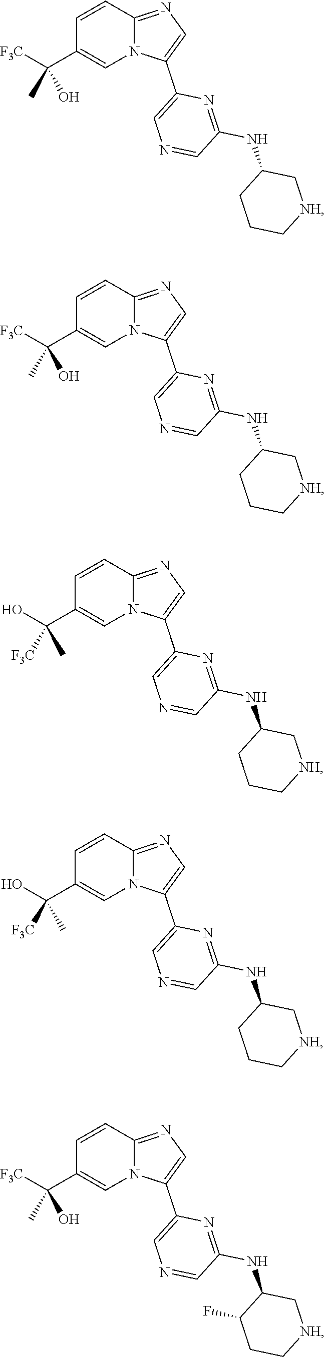 [SMILES: C[C@](O)(c1ccc2ncc(-c3cncc(N[C@H]4CCCNC4)n3)n2c1)C(F)(F)F ; C[C@@](O)(c1ccc2ncc(-c3cncc(N[C@H]4CCCNC4)n3)n2c1)C(F)(F)F ; C[C@@](O)(c1ccc2ncc(-c3cncc(N[C@@H]4CCCNC4)n3)n2c1)C(F)(F)F ; C[C@](O)(c1ccc2ncc(-c3cncc(N[C@@H]4CCCNC4)n3)n2c1)C(F)(F)F ; C[C@@](O)(c1ccc2ncc(-c3cncc(N[C@H]4CNCC[C@@H]4F)n3)n2c1)C(F)(F)F]

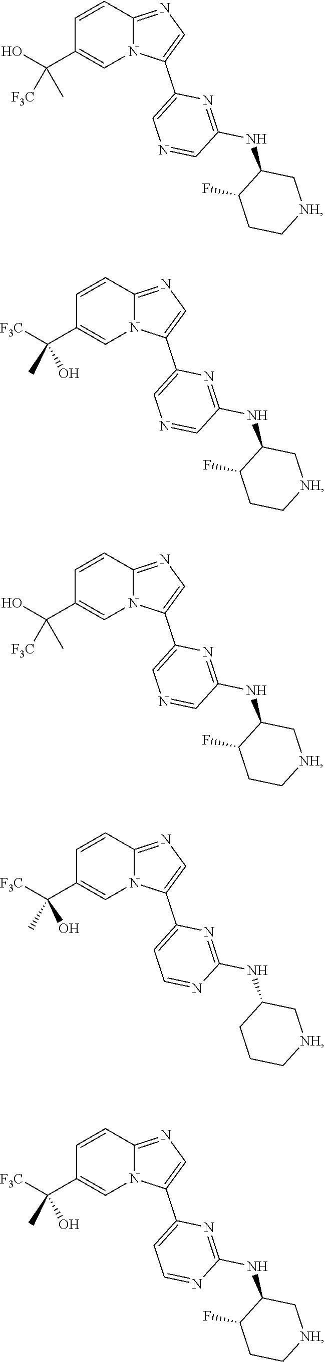 [SMILES: CC(O)(c1ccc2ncc(-c3cncc(N[C@H]4CNCC[C@@H]4F)n3)n2c1)C(F)(F)F ; C[C@](O)(c1ccc2ncc(-c3cncc(N[C@H]4CNCC[C@@H]4F)n3)n2c1)C(F)(F)F ; CC(O)(c1ccc2ncc(-c3cncc(N[C@H]4CNCC[C@@H]4F)n3)n2c1)C(F)(F)F ; C[C@@](O)(c1ccc2ncc(-c3ccnc(N[C@H]4CCCNC4)n3)n2c1)C(F)(F)F ; C[C@](O)(c1ccc2ncc(-c3ccnc(N[C@H]4CNCC[C@@H]4F)n3)n2c1)C(F)(F)F]

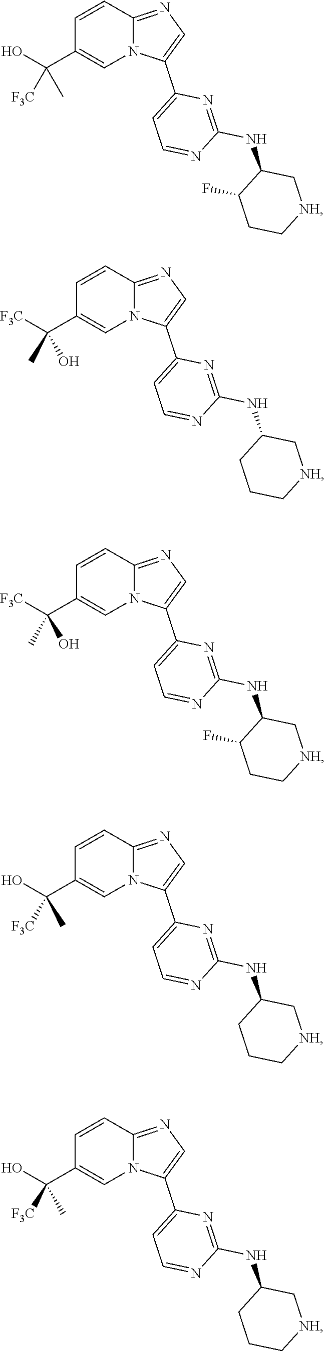 [SMILES: CC(O)(c1ccc2ncc(-c3ccnc(N[C@H]4CNCC[C@@H]4F)n3)n2c1)C(F)(F)F ; C[C@](O)(c1ccc2ncc(-c3ccnc(N[C@H]4CCCNC4)n3)n2c1)C(F)(F)F ; C[C@@](O)(c1ccc2ncc(-c3ccnc(N[C@H]4CNCC[C@@H]4F)n3)n2c1)C(F)(F)F ; C[C@@](O)(c1ccc2ncc(-c3ccnc(N[C@@H]4CCCNC4)n3)n2c1)C(F)(F)F ; C[C@](O)(c1ccc2ncc(-c3ccnc(N[C@@H]4CCCNC4)n3)n2c1)C(F)(F)F]

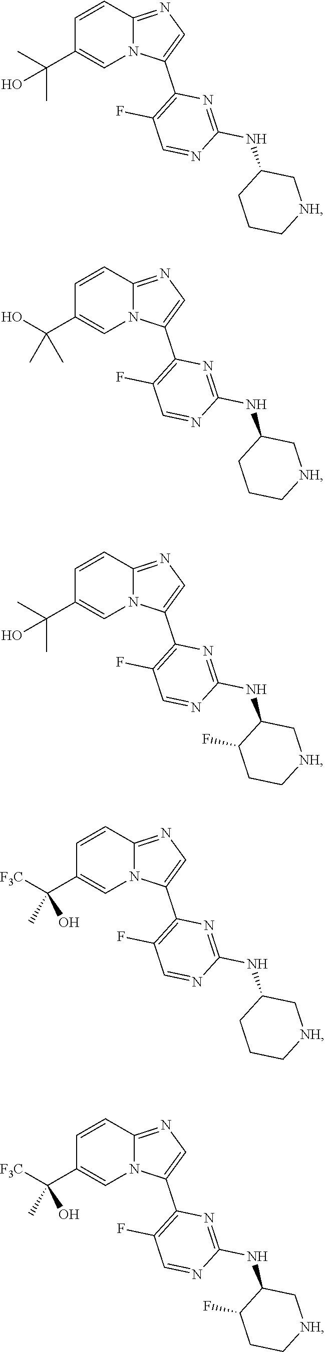 [SMILES: CC(C)(O)c1ccc2ncc(-c3nc(N[C@H]4CCCNC4)ncc3F)n2c1 ; CC(C)(O)c1ccc2ncc(-c3nc(N[C@@H]4CCCNC4)ncc3F)n2c1 ; CC(C)(O)c1ccc2ncc(-c3nc(N[C@H]4CNCC[C@@H]4F)ncc3F)n2c1 ; C[C@@](O)(c1ccc2ncc(-c3nc(N[C@H]4CCCNC4)ncc3F)n2c1)C(F)(F)F ; C[C@@](O)(c1ccc2ncc(-c3nc(N[C@H]4CNCC[C@@H]4F)ncc3F)n2c1)C(F)(F)F]

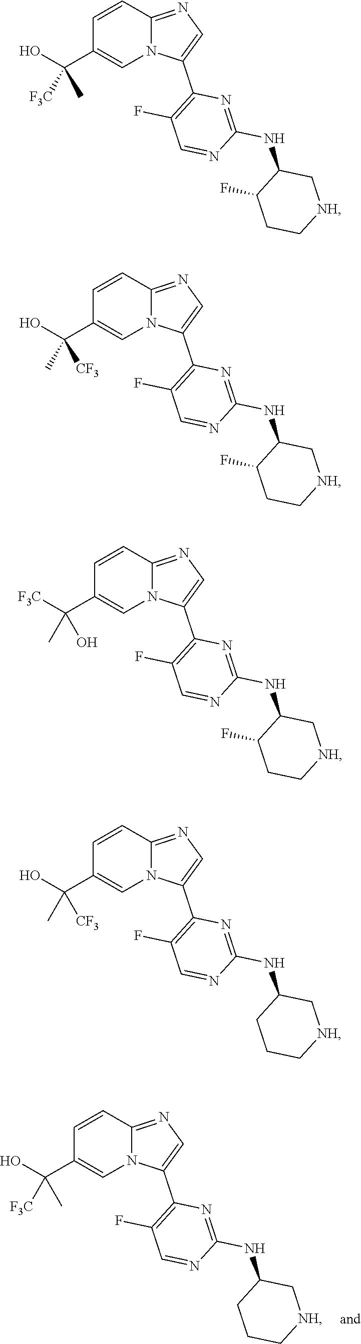 [SMILES: C[C@@](O)(c1ccc2ncc(-c3nc(N[C@H]4CNCC[C@@H]4F)ncc3F)n2c1)C(F)(F)F ; C[C@](O)(c1ccc2ncc(-c3nc(N[C@H]4CNCC[C@@H]4F)ncc3F)n2c1)C(F)(F)F ; CC(O)(c1ccc2ncc(-c3nc(N[C@H]4CNCC[C@@H]4F)ncc3F)n2c1)C(F)(F)F ; CC(O)(c1ccc2ncc(-c3nc(N[C@@H]4CCCNC4)ncc3F)n2c1)C(F)(F)F ; CC(O)(c1ccc2ncc(-c3nc(N[C@@H]4CCCNC4)ncc3F)n2c1)C(F)(F)F ; "and"]

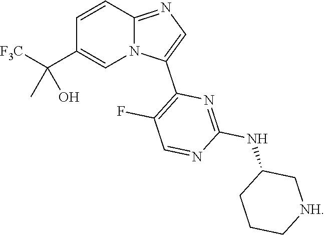 [SMILES: CC(O)(c1ccc2ncc(-c3nc(N[C@H]4CCCNC4)ncc3F)n2c1)C(F)(F)F]

Clause 113. The compound of clause 101 or 102, wherein the compound of Formula is a compound of Formula (IIa-5010-WO50):

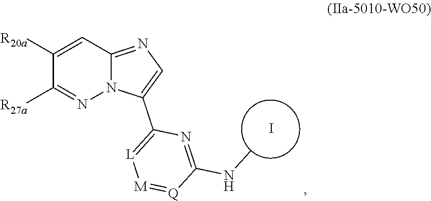

or a salt, ester, solvate, optical isomer, geometric isomer, or salt of an isomer thereof;
wherein: L is N or CR₂₁; M is N or CR₂₂; Q is N or CR₂₃;

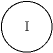

is

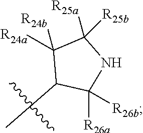

R₂₀ₐ is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from C₁-C₆ alkyl and halogen; R₂₇ₐ is selected from halogen, C₁-C₆ alkyl, C₁-C₆ alkoxy, C₃-C₆ cycloalkyl, spiro-fused cycloalkyl, —O—(C₃-C₆ cycloalkyl), C₂-C₆ heterocyclyl, imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl, spiro-cycloalkyl, —O—(C₃-C₆ cycloalkyl), and C₂-C₆ heterocyclyl are each optionally substituted with one or more substituents selected from —OH, C₁-C₆ alkyl, and halogen; R₂₁, R₂₂, and R₂₃ are each independently selected from H, C₁-C₆ alkoxy, and halogen; R₂₄ₐ, R_{24b}, R₂₅ₐ, R_{25b}, R₂₆ₐ, and R_{26b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R₂₈ₐ and R_{28b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of L, M, or Q is N.

Clause 114. The compound of clause 113, wherein at least one of (i)-(v) applies: (i) each of R_{24b}, R₂₅ₐ, R_{25b}, R₂₆ₐ, and R_{26b} is H and R₂₄ₐ is F; (ii) R₂₁, R₂₂, and R₂₃, if present, are H; (iii) L is CR₂₁ wherein R₂₁ is selected from F, Cl, and —OCH₃, M is CR₂₂ wherein R₂₂ is H, and Q is N; (iv) R₂₀ₐ is selected from —OCH₃, unsubstituted —O—(C₃ cycloalkyl),

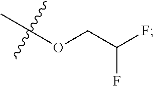 [SMILES: *OCC(F)F]

and
(v) R₂₇ₐ is selected from unsubstituted C₃-C₆ cycloalkyl,

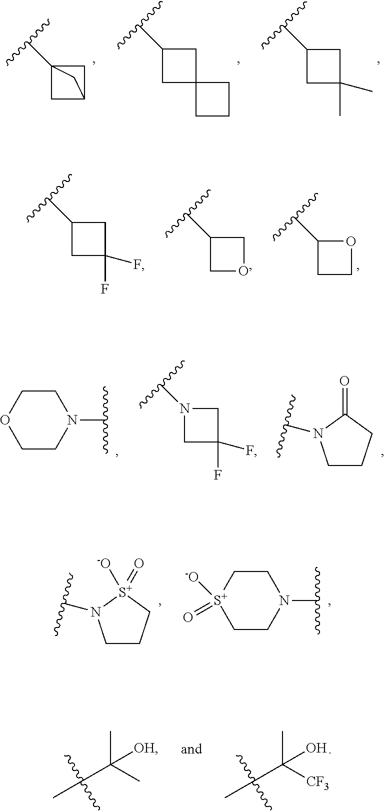

Clause 115. The compound of clause 113 or 114, with the proviso that: when R₂₀ₐ is —OCH₃ and R₂₇ₐ is unsubstituted C₃ cycloalkyl, at least one of R₂₁, R₂₂, and R₂₃ is present and selected from C₁-C₆ alkoxy and halogen.

Clause 116. The compound of any one of clauses 113 to 115, wherein the compound of Formula (IIa-5010-WO50) is selected from:

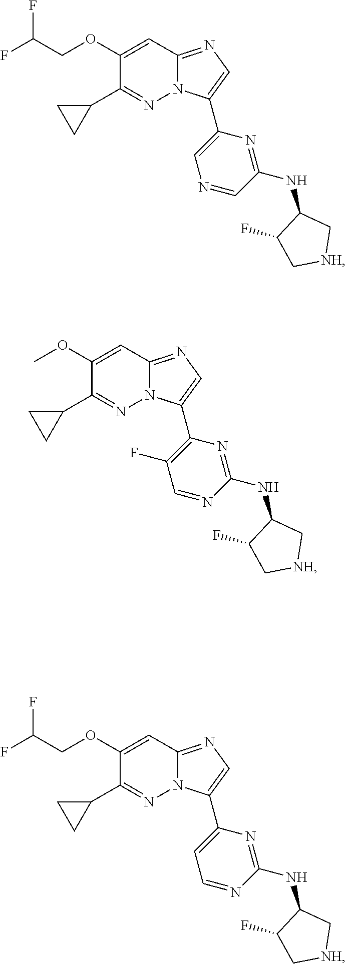 [SMILES: FC(F)COc1cc2ncc(-c3cncc(N[C@H]4CNC[C@@H]4F)n3)n2nc1C1CC1 ; COc1cc2ncc(-c3nc(N[C@H]4CNC[C@@H]4F)ncc3F)n2nc1C1CC1 ; FC(F)COc1cc2ncc(-c3ccnc(N[C@H]4CNC[C@@H]4F)n3)n2nc1C1CC1]

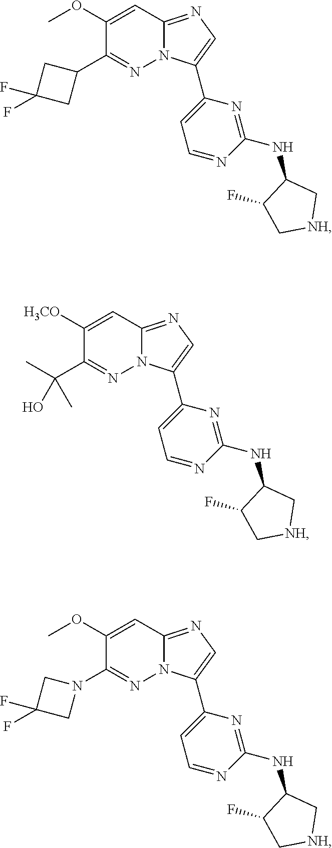 [SMILES: COc1cc2ncc(-c3ccnc(N[C@H]4CNC[C@@H]4F)n3)n2nc1C1CC(F)(F)C1 ; COc1cc2ncc(-c3ccnc(N[C@H]4CNC[C@@H]4F)n3)n2nc1C(C)(C)O ; COc1cc2ncc(-c3ccnc(N[C@H]4CNC[C@@H]4F)n3)n2nc1N1CC(F)(F)C1]

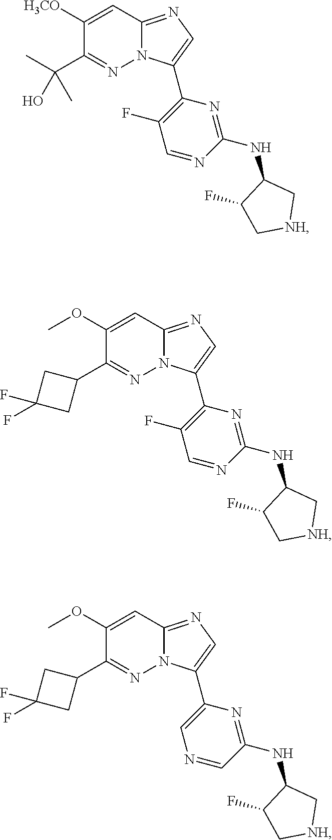 [SMILES: COc1cc2ncc(-c3nc(N[C@H]4CNC[C@@H]4F)ncc3F)n2nc1C(C)(C)O ; COc1cc2ncc(-c3nc(N[C@H]4CNC[C@@H]4F)ncc3F)n2nc1C1CC(F)(F)C1 ; COc1cc2ncc(-c3cncc(N[C@H]4CNC[C@@H]4F)n3)n2nc1C1CC(F)(F)C1]

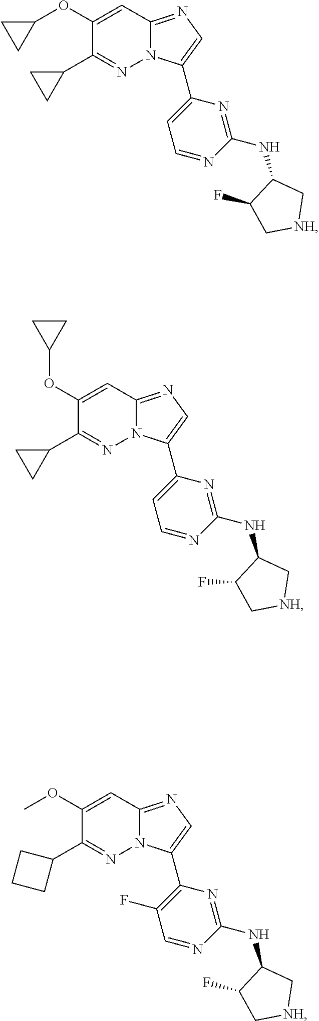 [SMILES: F[C@@H]1CNC[C@H]1Nc1nccc(-c2cnc3cc(OC4CC4)c(C4CC4)nn23)n1 ; F[C@H]1CNC[C@@H]1Nc1nccc(-c2cnc3cc(OC4CC4)c(C4CC4)nn23)n1 ; COc1cc2ncc(-c3nc(N[C@H]4CNC[C@@H]4F)ncc3F)n2nc1C1CCC1]

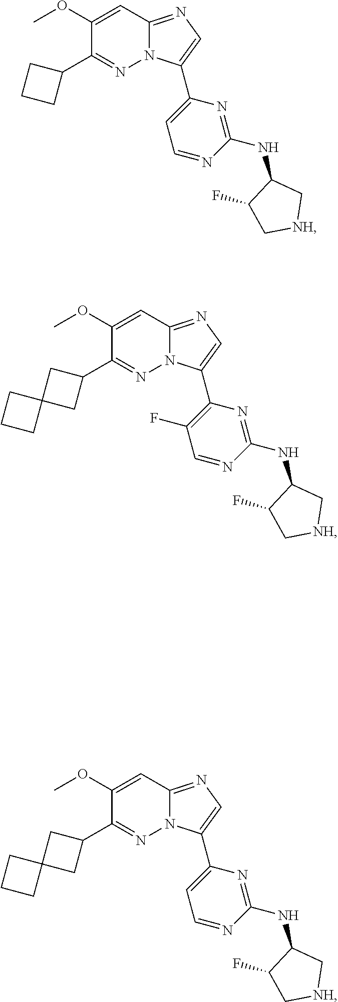 [SMILES: COc1cc2ncc(-c3ccnc(N[C@H]4CNC[C@@H]4F)n3)n2nc1C1CCC1 ; COc1cc2ncc(-c3nc(N[C@H]4CNC[C@@H]4F)ncc3F)n2nc1C1CC2(CCC2)C1 ; COc1cc2ncc(-c3ccnc(N[C@H]4CNC[C@@H]4F)n3)n2nc1C1CC2(CCC2)C1]

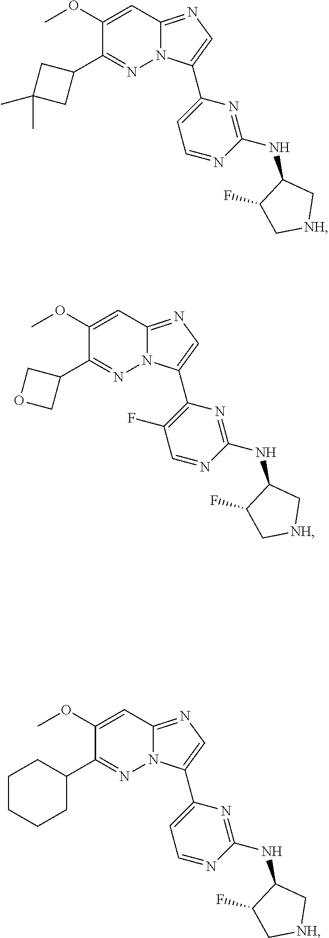 [SMILES: COc1cc2ncc(-c3ccnc(N[C@H]4CNC[C@@H]4F)n3)n2nc1C1CC(C)(C)C1 ; COc1cc2ncc(-c3nc(N[C@H]4CNC[C@@H]4F)ncc3F)n2nc1C1COC1 ; COc1cc2ncc(-c3ccnc(N[C@H]4CNC[C@@H]4F)n3)n2nc1C1CCCCC1]

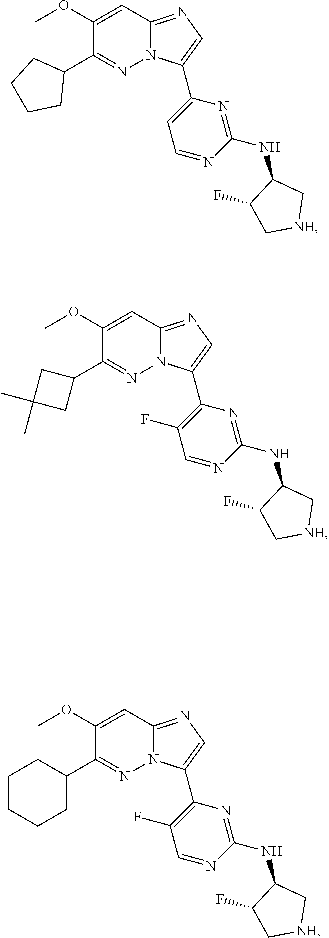 [SMILES: COc1cc2ncc(-c3ccnc(N[C@H]4CNC[C@@H]4F)n3)n2nc1C1CCCC1 ; COc1cc2ncc(-c3nc(N[C@H]4CNC[C@@H]4F)ncc3F)n2nc1C1CC(C)(C)C1 ; COc1cc2ncc(-c3nc(N[C@H]4CNC[C@@H]4F)ncc3F)n2nc1C1CCCCC1]

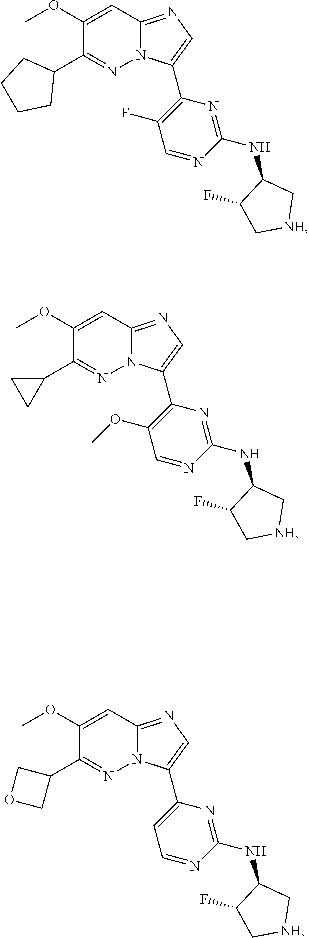 [SMILES: COc1cc2ncc(-c3nc(N[C@H]4CNC[C@@H]4F)ncc3F)n2nc1C1CCCC1 ; COc1cnc(N[C@H]2CNC[C@@H]2F)nc1-c1cnc2cc(OC)c(C3CC3)nn12 ; COc1cc2ncc(-c3ccnc(N[C@H]4CNC[C@@H]4F)n3)n2nc1C1COC1]

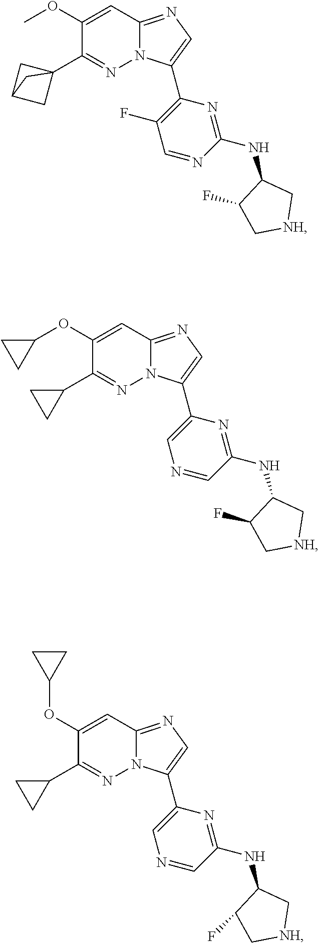 [SMILES: COc1cc2ncc(-c3nc(N[C@H]4CNC[C@@H]4F)ncc3F)n2nc1C12CC(C1)C2 ; F[C@@H]1CNC[C@H]1Nc1cncc(-c2cnc3cc(OC4CC4)c(C4CC4)nn23)n1 ; F[C@H]1CNC[C@@H]1Nc1cncc(-c2cnc3cc(OC4CC4)c(C4CC4)nn23)n1]

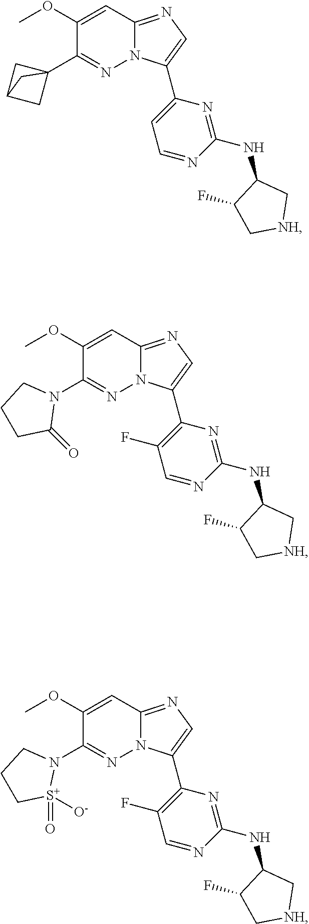 [SMILES: COc1cc2ncc(-c3ccnc(N[C@H]4CNC[C@@H]4F)n3)n2nc1C12CC(C1)C2 ; COc1cc2ncc(-c3nc(N[C@H]4CNC[C@@H]4F)ncc3F)n2nc1N1CCCC1=O ; COc1cc2ncc(-c3nc(N[C@H]4CNC[C@@H]4F)ncc3F)n2nc1N1CCC[S+]1(=O)[O-]]

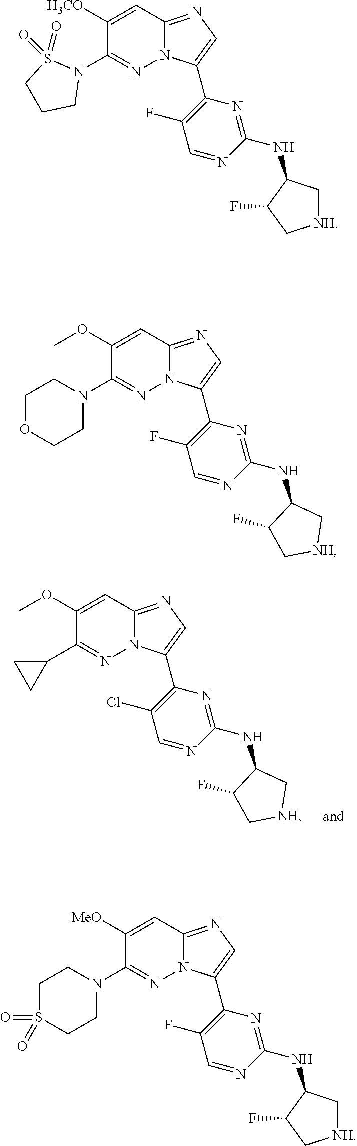 [SMILES: COc1cc2ncc(-c3nc(N[C@H]4CNC[C@@H]4F)ncc3F)n2nc1N1CCCS1(=O)=O ; COc1cc2ncc(-c3nc(N[C@H]4CNC[C@@H]4F)ncc3F)n2nc1N1CCOCC1 ; COc1cc2ncc(-c3nc(N[C@H]4CNC[C@@H]4F)ncc3Cl)n2nc1C1CC1 ; COc1cc2ncc(-c3nc(N[C@H]4CNC[C@@H]4F)ncc3F)n2nc1N1CCS(=O)(=O)CC1]

Clause 17. The compound of clause 101 or 102, wherein the compound of Formula (II) is a compound of Formula (IIb-5010-WO50):

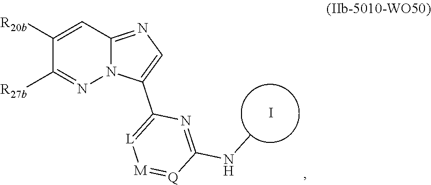

or a salt, ester, solvate, optical isomer, geometric isomer, or salt of an isomer thereof;
wherein: L is N or CR₂₁; M is N or CR₂₂; Q is N or CR₂₃;

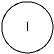

is

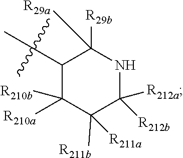

R_{20b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from C₁-C₆ alkyl and halogen; R_{27b} is selected from halogen, C₁-C₆ alkyl, C₁-C₆ alkoxy, C₃-C₆ cycloalkyl, spiro-cycloalkyl, —O—(C₃-C₆ cycloalkyl), C₂-C₆ heterocyclyl, imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl, spiro-cycloalkyl, —O—(C₃-C₆ cycloalkyl), and C₂-C₆ heterocyclyl are each optionally substituted with one or more substituents selected from —OH, C₁-C₆ alkyl, and halogen; R₂₁, R₂₂, and R₂₃ are each independently selected from H, C₁-C₆ alkoxy, and halogen; R₂₉ₐ, R_{29b}, R₂₁₀ₐ, R_{210b}, R_{210b}, R_{211b}, R₂₁₂ₐ, and R_{212b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R₂₈ₐ and R_{28b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of L, M, or Q is N.

Clause 118. The compound of clause 117, wherein at least one of (i)-(vi) applies: (i) each of R₂₉ₐ, R_{29b}, R₂₁₀ₐ, R_{210b}, R₂₁₁ₐ, R_{211b}, R₂₁₂ₐ, and R_{212b} is H; (ii) each of R₂₉ₐ, R_{29b}, R_{210b}, R₂₁₁ₐ, R_{211b}, R₂₁₂ₐ, and R_{212b} is H and R₂₁₀ₐ is F; (iii) R₂₁, R₂₂, and R₂₃, if present, are H; (iv) L is CR₂₁ wherein R₂₁ is selected from F, Cl, and —OCH₃, M is CR₂₂ wherein R₂₂ is H, and Q is N; (v) R_{20b} is selected from —OCH₃, —OCD₃, unsubstituted —O—(C₃ cycloalkyl), and

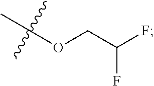 [SMILES: *OCC(F)F]

and (vi) R_{27b} is selected from unsubstituted C₃-C₆ cycloalkyl,

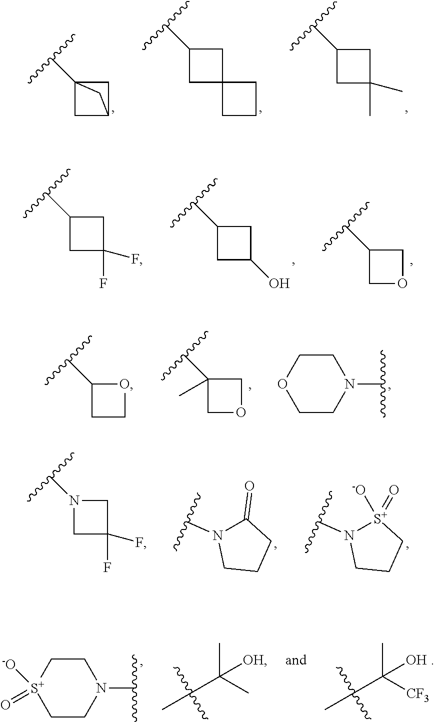

Clause 119. The compound of clause 117 or 118, with the proviso that: when R_{20b} is —OCH₃ and R_{27b} is unsubstituted C₃ cycloalkyl, at least one of R₂₁, R₂₂, and R₂₃ is present and selected from C₁-C₆ alkoxy and halogen.

Clause 120. The compound of any one of clauses 117 to 119, wherein the compound of Formula (IIb-5010-WO50) is selected from:

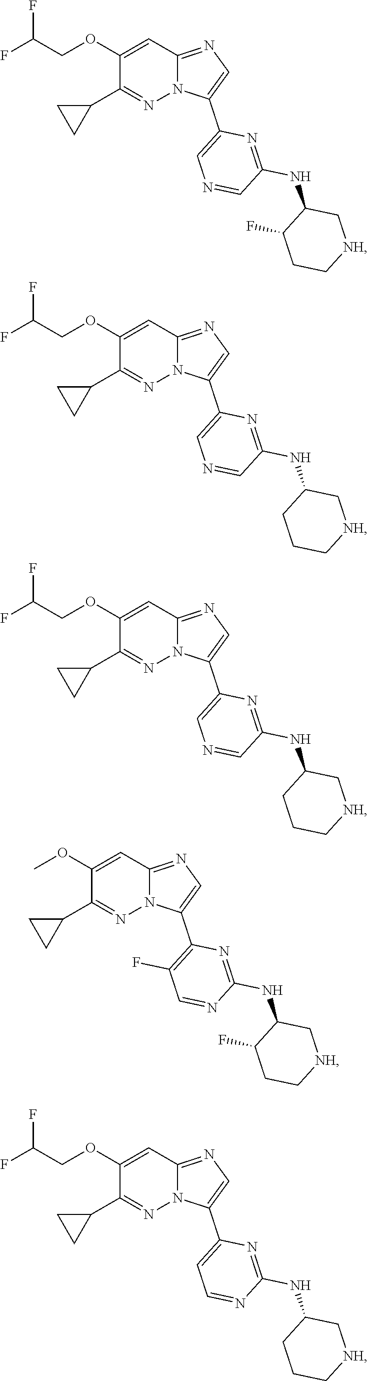 [SMILES: FC(F)COc1cc2ncc(-c3cncc(N[C@H]4CNCC[C@@H]4F)n3)n2nc1C1CC1 ; FC(F)COc1cc2ncc(-c3cncc(N[C@H]4CCCNC4)n3)n2nc1C1CC1 ; FC(F)COc1cc2ncc(-c3cncc(N[C@@H]4CCCNC4)n3)n2nc1C1CC1 ; COc1cc2ncc(-c3nc(N[C@H]4CNCC[C@@H]4F)ncc3F)n2nc1C1CC1 ; FC(F)COc1cc2ncc(-c3ccnc(N[C@H]4CCCNC4)n3)n2nc1C1CC1]

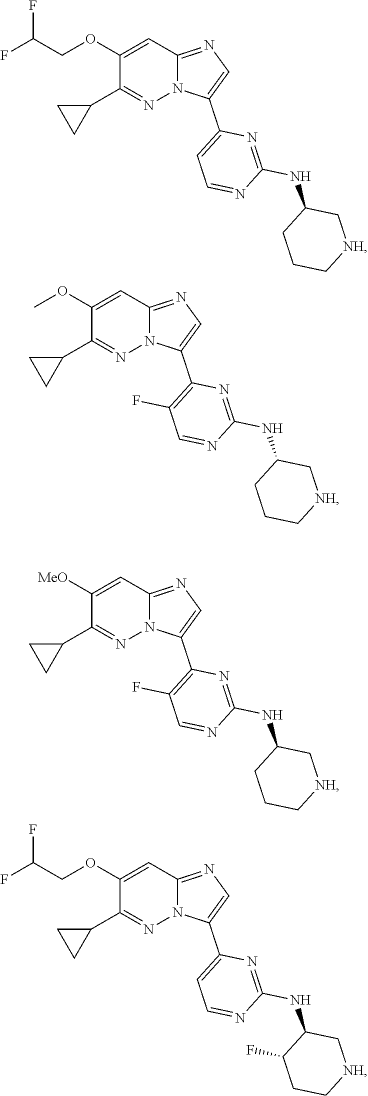 [SMILES: FC(F)COc1cc2ncc(-c3ccnc(N[C@@H]4CCCNC4)n3)n2nc1C1CC1 ; COc1cc2ncc(-c3nc(N[C@H]4CCCNC4)ncc3F)n2nc1C1CC1 ; COc1cc2ncc(-c3nc(N[C@@H]4CCCNC4)ncc3F)n2nc1C1CC1 ; FC(F)COc1cc2ncc(-c3ccnc(N[C@H]4CNCC[C@@H]4F)n3)n2nc1C1CC1]

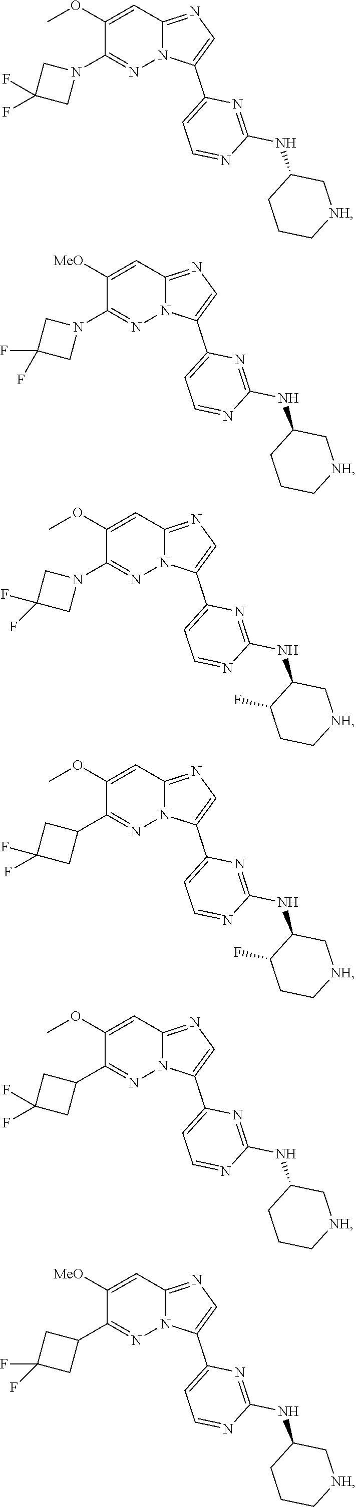 [SMILES: COc1cc2ncc(-c3ccnc(N[C@H]4CCCNC4)n3)n2nc1N1CC(F)(F)C1 ; COc1cc2ncc(-c3ccnc(N[C@@H]4CCCNC4)n3)n2nc1N1CC(F)(F)C1 ; COc1cc2ncc(-c3ccnc(N[C@H]4CNCC[C@@H]4F)n3)n2nc1N1CC(F)(F)C1 ; COc1cc2ncc(-c3ccnc(N[C@H]4CNCC[C@@H]4F)n3)n2nc1C1CC(F)(F)C1 ; COc1cc2ncc(-c3ccnc(N[C@H]4CCCNC4)n3)n2nc1C1CC(F)(F)C1 ; COc1cc2ncc(-c3ccnc(N[C@@H]4CCCNC4)n3)n2nc1C1CC(F)(F)C1]

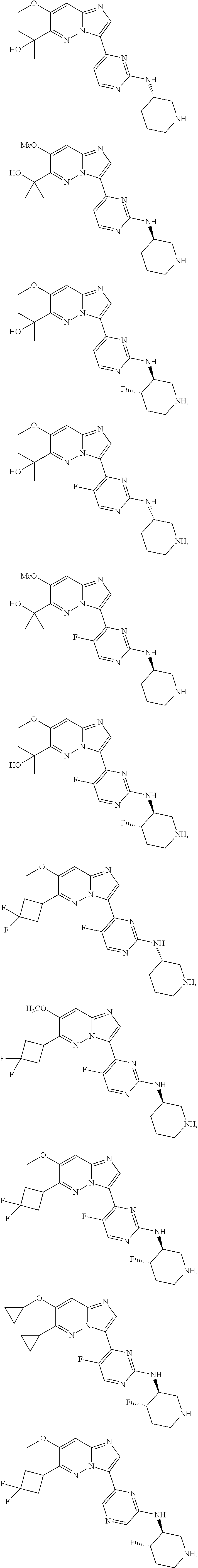

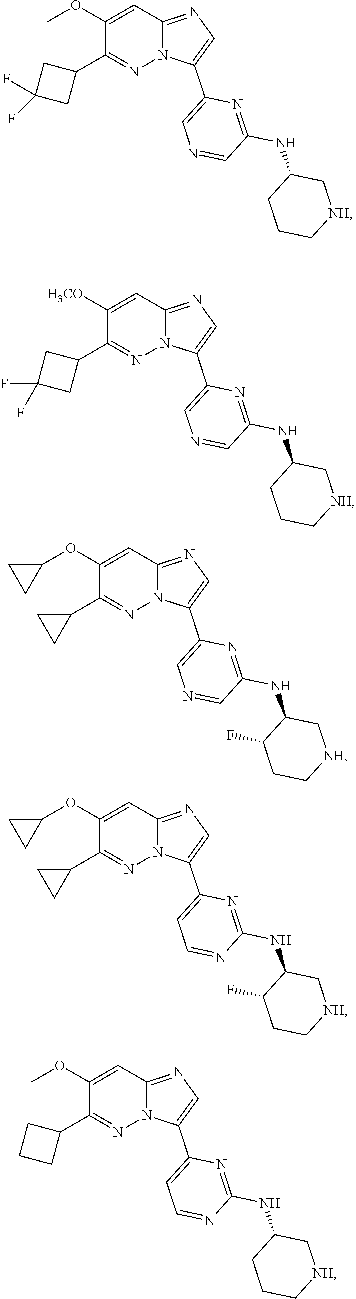 [SMILES: COc1cc2ncc(-c3cncc(N[C@H]4CCCNC4)n3)n2nc1C1CC(F)(F)C1 ; COc1cc2ncc(-c3cncc(N[C@@H]4CCCNC4)n3)n2nc1C1CC(F)(F)C1 ; F[C@H]1CCNC[C@@H]1Nc1cncc(-c2cnc3cc(OC4CC4)c(C4CC4)nn23)n1 ; F[C@H]1CCNC[C@@H]1Nc1nccc(-c2cnc3cc(OC4CC4)c(C4CC4)nn23)n1 ; COc1cc2ncc(-c3ccnc(N[C@H]4CCCNC4)n3)n2nc1C1CCC1]

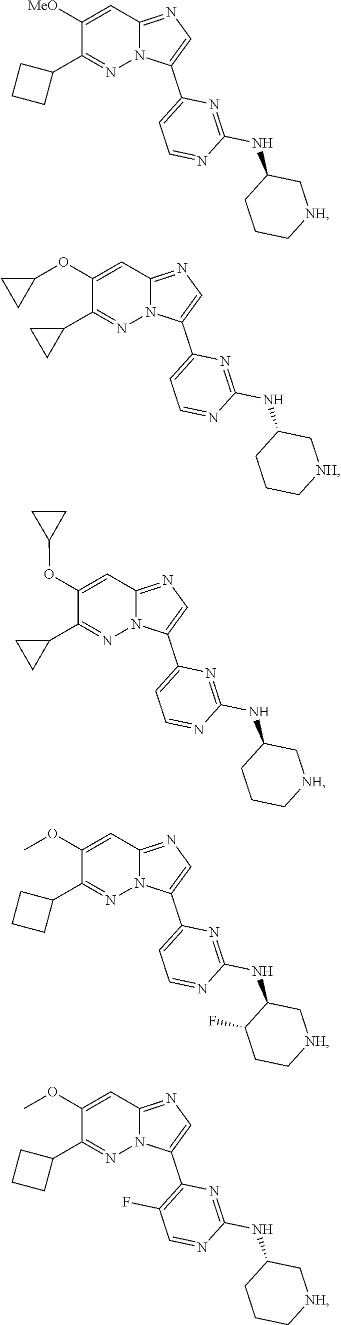 [SMILES: COc1cc2ncc(-c3ccnc(N[C@@H]4CCCNC4)n3)n2nc1C1CCC1 ; c1cc(-c2cnc3cc(OC4CC4)c(C4CC4)nn23)nc(N[C@H]2CCCNC2)n1 ; c1cc(-c2cnc3cc(OC4CC4)c(C4CC4)nn23)nc(N[C@@H]2CCCNC2)n1 ; COc1cc2ncc(-c3ccnc(N[C@H]4CNCC[C@@H]4F)n3)n2nc1C1CCC1 ; COc1cc2ncc(-c3nc(N[C@H]4CCCNC4)ncc3F)n2nc1C1CCC1]

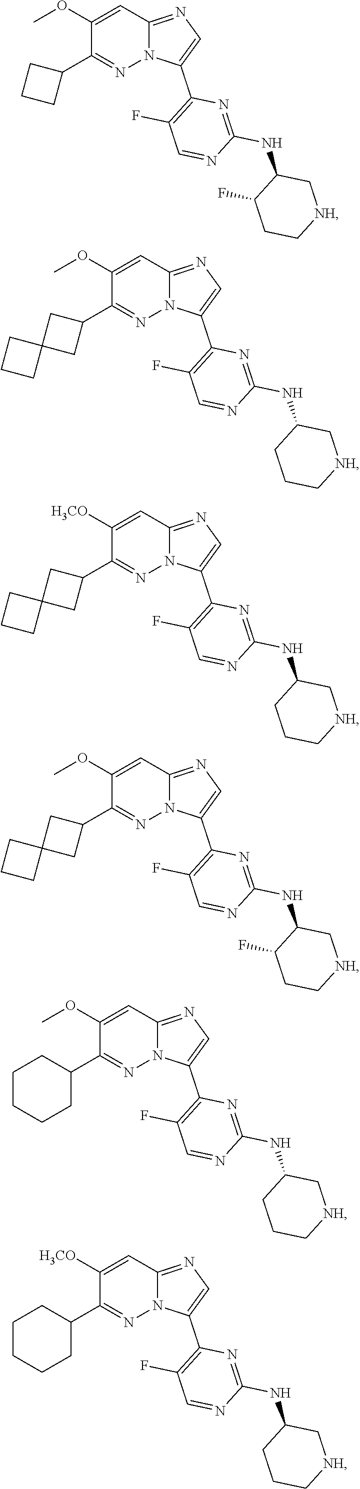 [SMILES: COc1cc2ncc(-c3nc(N[C@H]4CNCC[C@@H]4F)ncc3F)n2nc1C1CCC1 ; COc1cc2ncc(-c3nc(N[C@H]4CCCNC4)ncc3F)n2nc1C1CC2(CCC2)C1 ; COc1cc2ncc(-c3nc(N[C@@H]4CCCNC4)ncc3F)n2nc1C1CC2(CCC2)C1 ; COc1cc2ncc(-c3nc(N[C@H]4CNCC[C@@H]4F)ncc3F)n2nc1C1CC2(CCC2)C1 ; COc1cc2ncc(-c3nc(N[C@H]4CCCNC4)ncc3F)n2nc1C1CCCCC1 ; COc1cc2ncc(-c3nc(N[C@@H]4CCCNC4)ncc3F)n2nc1C1CCCCC1]

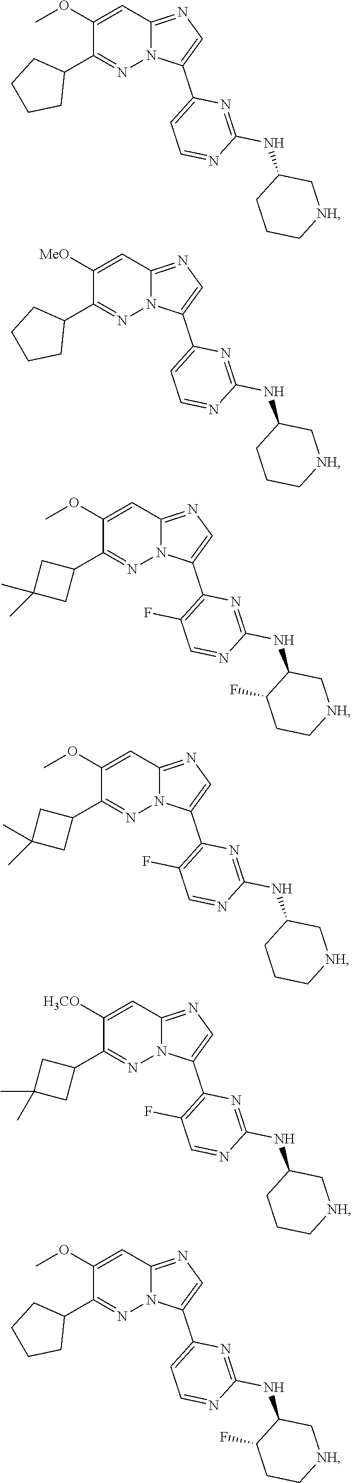 [SMILES: COc1cc2ncc(-c3ccnc(N[C@H]4CCCNC4)n3)n2nc1C1CCCC1 ; COc1cc2ncc(-c3ccnc(N[C@@H]4CCCNC4)n3)n2nc1C1CCCC1 ; COc1cc2ncc(-c3nc(N[C@H]4CNCC[C@@H]4F)ncc3F)n2nc1C1CC(C)(C)C1 ; COc1cc2ncc(-c3nc(N[C@H]4CCCNC4)ncc3F)n2nc1C1CC(C)(C)C1 ; COc1cc2ncc(-c3nc(N[C@@H]4CCCNC4)ncc3F)n2nc1C1CC(C)(C)C1 ; COc1cc2ncc(-c3ccnc(N[C@H]4CNCC[C@@H]4F)n3)n2nc1C1CCCC1]

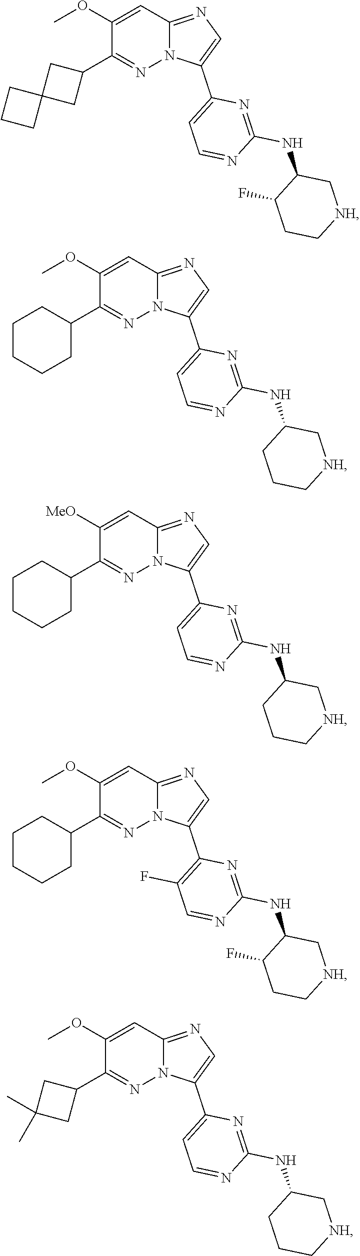 [SMILES: COc1cc2ncc(-c3ccnc(N[C@H]4CNCC[C@@H]4F)n3)n2nc1C1CC2(CCC2)C1 ; COc1cc2ncc(-c3ccnc(N[C@H]4CCCNC4)n3)n2nc1C1CCCCC1 ; COc1cc2ncc(-c3ccnc(N[C@@H]4CCCNC4)n3)n2nc1C1CCCCC1 ; COc1cc2ncc(-c3nc(N[C@H]4CNCC[C@@H]4F)ncc3F)n2nc1C1CCCCC1 ; COc1cc2ncc(-c3ccnc(N[C@H]4CCCNC4)n3)n2nc1C1CC(C)(C)C1]

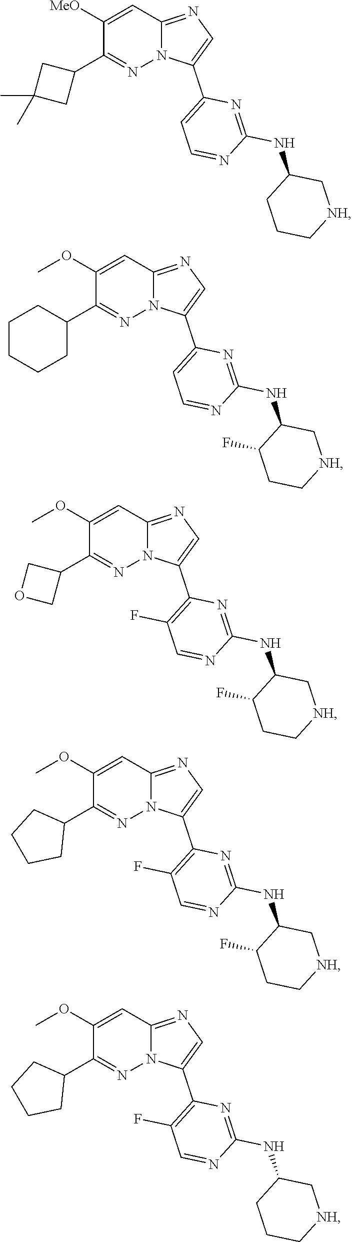 [SMILES: COc1cc2ncc(-c3ccnc(N[C@@H]4CCCNC4)n3)n2nc1C1CC(C)(C)C1 ; COc1cc2ncc(-c3ccnc(N[C@H]4CNCC[C@@H]4F)n3)n2nc1C1CCCCC1 ; COc1cc2ncc(-c3nc(N[C@H]4CNCC[C@@H]4F)ncc3F)n2nc1C1COC1 ; COc1cc2ncc(-c3nc(N[C@H]4CNCC[C@@H]4F)ncc3F)n2nc1C1CCCC1 ; COc1cc2ncc(-c3nc(N[C@H]4CCCNC4)ncc3F)n2nc1C1CCCC1]

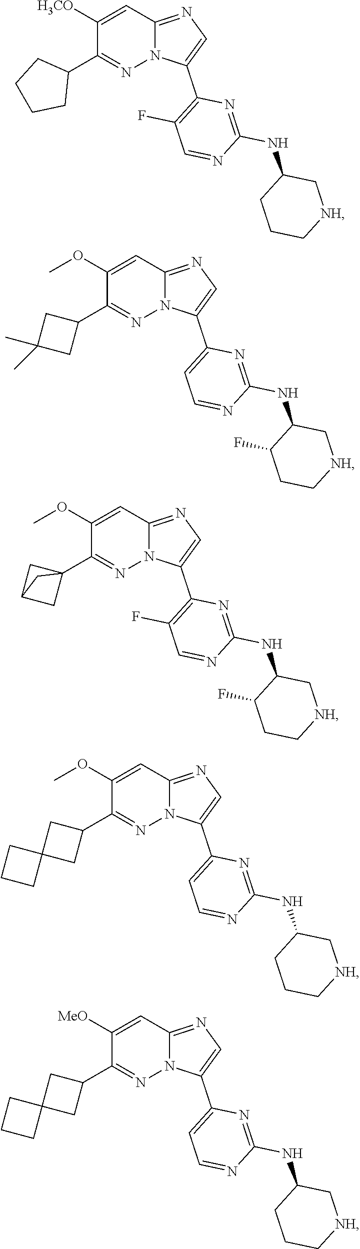 [SMILES: COc1cc2ncc(-c3nc(N[C@@H]4CCCNC4)ncc3F)n2nc1C1CCCC1 ; COc1cc2ncc(-c3ccnc(N[C@H]4CNCC[C@@H]4F)n3)n2nc1C1CC(C)(C)C1 ; COc1cc2ncc(-c3nc(N[C@H]4CNCC[C@@H]4F)ncc3F)n2nc1C12CC(C1)C2 ; COc1cc2ncc(-c3ccnc(N[C@H]4CCCNC4)n3)n2nc1C1CC2(CCC2)C1 ; COc1cc2ncc(-c3ccnc(N[C@@H]4CCCNC4)n3)n2nc1C1CC2(CCC2)C1]

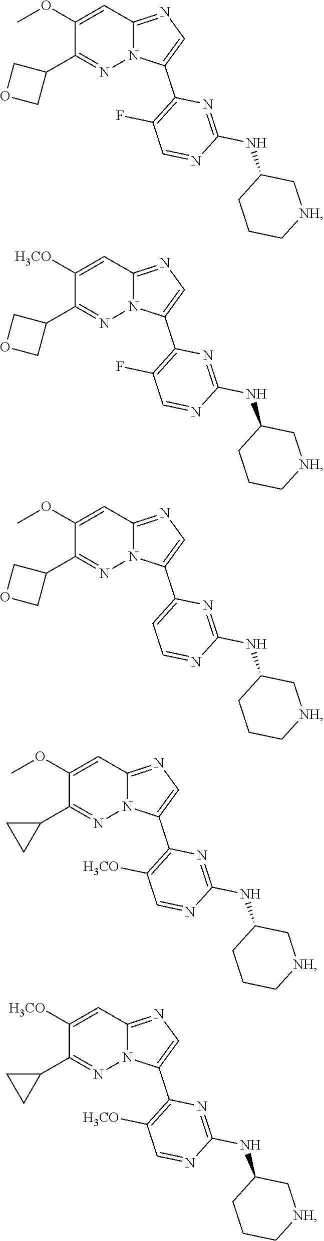 [SMILES: COc1cc2ncc(-c3nc(N[C@H]4CCCNC4)ncc3F)n2nc1C1COC1 ; COc1cc2ncc(-c3nc(N[C@@H]4CCCNC4)ncc3F)n2nc1C1COC1 ; COc1cc2ncc(-c3ccnc(N[C@H]4CCCNC4)n3)n2nc1C1COC1 ; COc1cnc(N[C@H]2CCCNC2)nc1-c1cnc2cc(OC)c(C3CC3)nn12 ; COc1cnc(N[C@@H]2CCCNC2)nc1-c1cnc2cc(OC)c(C3CC3)nn12]

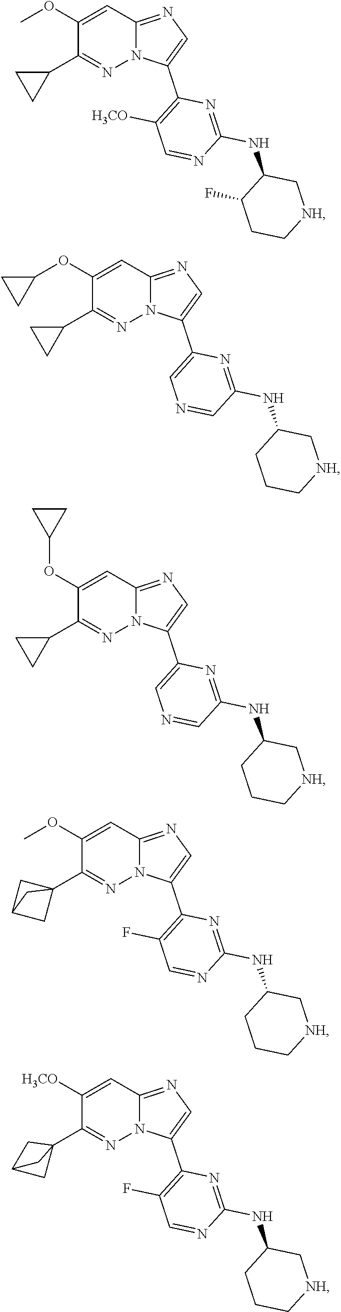 [SMILES: COc1cnc(N[C@H]2CNCC[C@@H]2F)nc1-c1cnc2cc(OC)c(C3CC3)nn12 ; c1ncc(-c2cnc3cc(OC4CC4)c(C4CC4)nn23)nc1N[C@H]1CCCNC1 ; c1ncc(-c2cnc3cc(OC4CC4)c(C4CC4)nn23)nc1N[C@@H]1CCCNC1 ; COc1cc2ncc(-c3nc(N[C@H]4CCCNC4)ncc3F)n2nc1C12CC(C1)C2 ; COc1cc2ncc(-c3nc(N[C@@H]4CCCNC4)ncc3F)n2nc1C12CC(C1)C2]

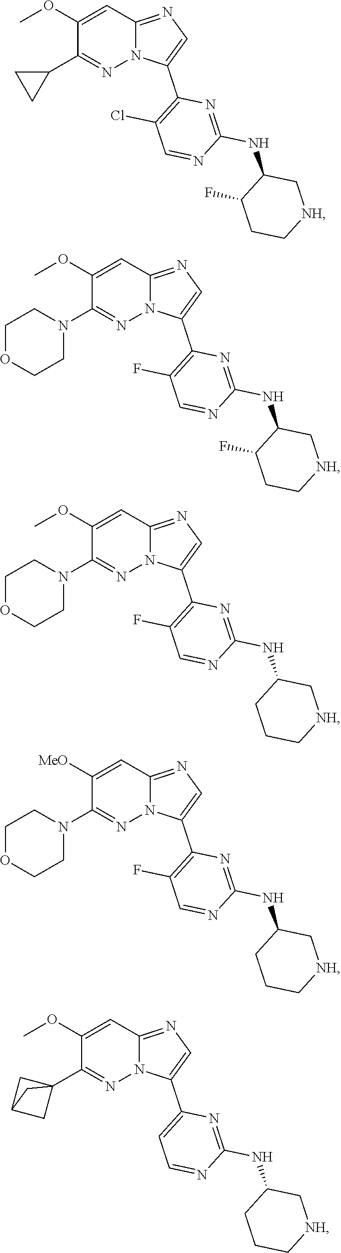 [SMILES: COc1cc2ncc(-c3nc(N[C@H]4CNCC[C@@H]4F)ncc3Cl)n2nc1C1CC1 ; COc1cc2ncc(-c3nc(N[C@H]4CNCC[C@@H]4F)ncc3F)n2nc1N1CCOCC1 ; COc1cc2ncc(-c3nc(N[C@H]4CCCNC4)ncc3F)n2nc1N1CCOCC1 ; COc1cc2ncc(-c3nc(N[C@@H]4CCCNC4)ncc3F)n2nc1N1CCOCC1 ; COc1cc2ncc(-c3ccnc(N[C@H]4CCCNC4)n3)n2nc1C12CC(C1)C2]

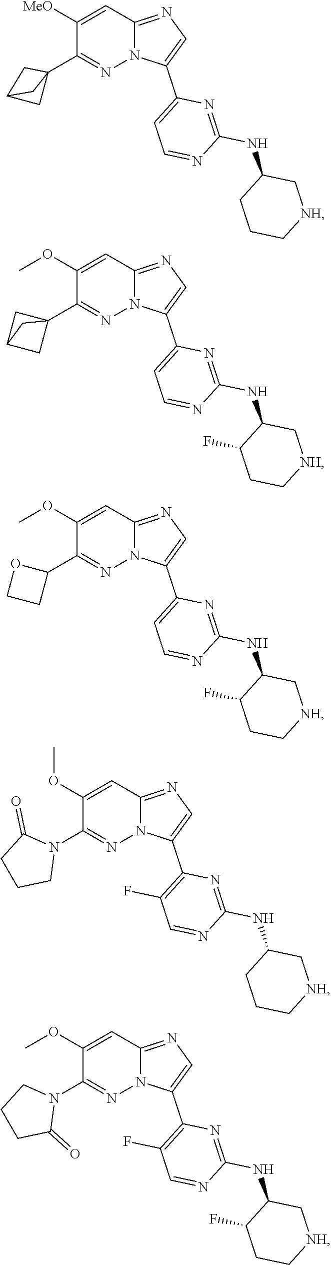 [SMILES: COc1cc2ncc(-c3ccnc(N[C@@H]4CCCNC4)n3)n2nc1C12CC(C1)C2 ; COc1cc2ncc(-c3ccnc(N[C@H]4CNCC[C@@H]4F)n3)n2nc1C12CC(C1)C2 ; COc1cc2ncc(-c3ccnc(N[C@H]4CNCC[C@@H]4F)n3)n2nc1C1CCO1 ; COc1cc2ncc(-c3nc(N[C@H]4CCCNC4)ncc3F)n2nc1N1CCCC1=O ; COc1cc2ncc(-c3nc(N[C@H]4CNCC[C@@H]4F)ncc3F)n2nc1N1CCCC1=O]

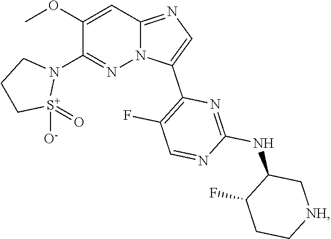 [SMILES: COc1cc2ncc(-c3nc(N[C@H]4CNCC[C@@H]4F)ncc3F)n2nc1N1CCC[S+]1(=O)[O-]]

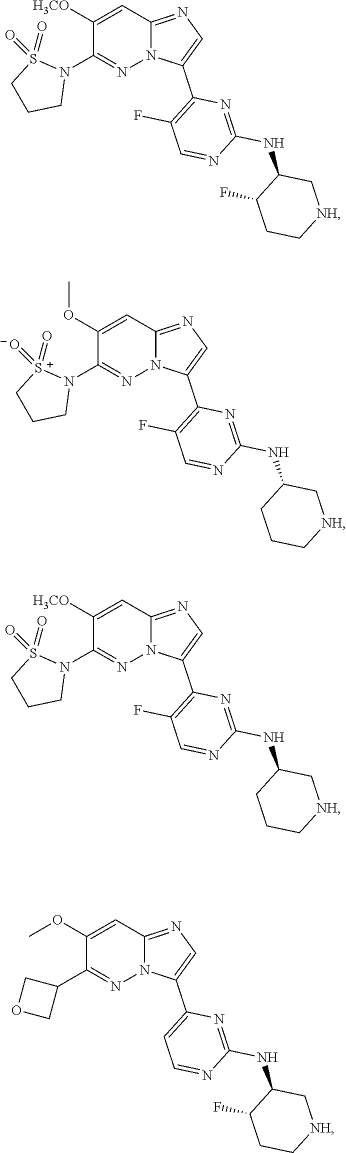

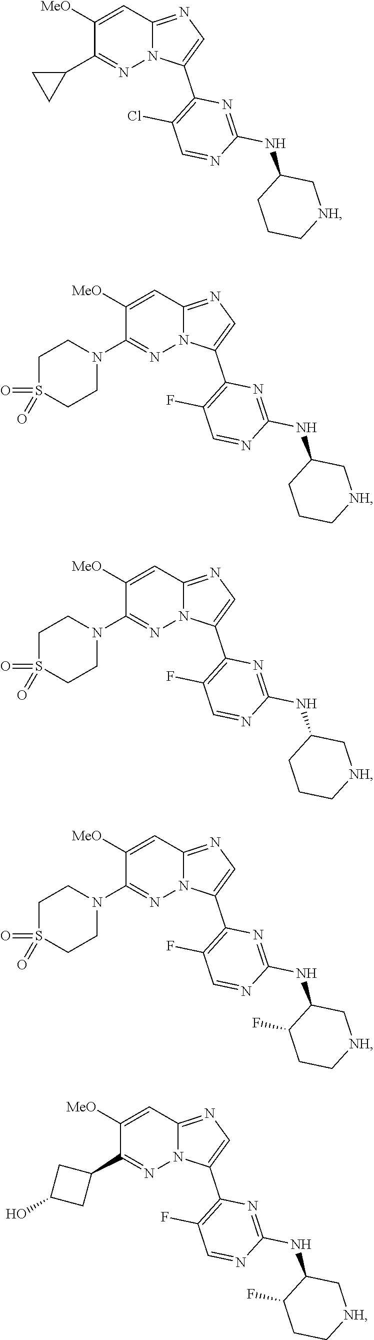 [SMILES: COc1cc2ncc(-c3nc(N[C@@H]4CCCNC4)ncc3Cl)n2nc1C1CC1 ; COc1cc2ncc(-c3nc(N[C@@H]4CCCNC4)ncc3F)n2nc1N1CCS(=O)(=O)CC1 ; COc1cc2ncc(-c3nc(N[C@H]4CCCNC4)ncc3F)n2nc1N1CCS(=O)(=O)CC1 ; COc1cc2ncc(-c3nc(N[C@H]4CNCC[C@@H]4F)ncc3F)n2nc1N1CCS(=O)(=O)CC1 ; COc1cc2ncc(-c3nc(N[C@H]4CNCC[C@@H]4F)ncc3F)n2nc1[C@H]1C[C@H](O)C1]

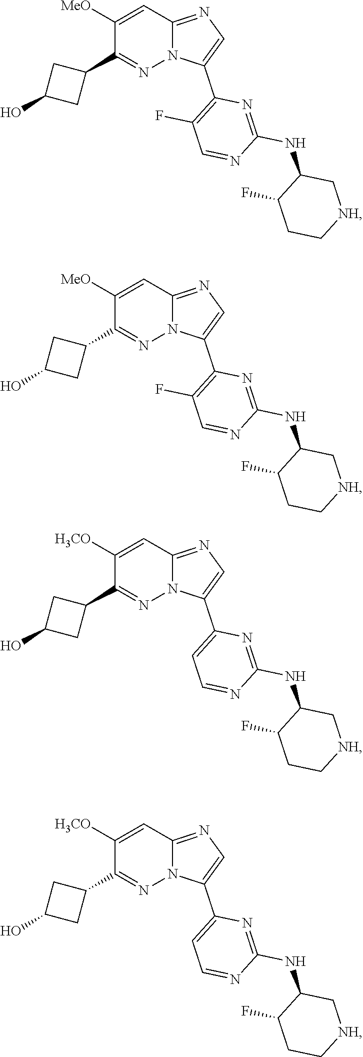 [SMILES: COc1cc2ncc(-c3nc(N[C@H]4CNCC[C@@H]4F)ncc3F)n2nc1[C@H]1C[C@@H](O)C1 ; COc1cc2ncc(-c3nc(N[C@H]4CNCC[C@@H]4F)ncc3F)n2nc1[C@H]1C[C@@H](O)C1 ; COc1cc2ncc(-c3ccnc(N[C@H]4CNCC[C@@H]4F)n3)n2nc1[C@H]1C[C@@H](O)C1 ; COc1cc2ncc(-c3ccnc(N[C@H]4CNCC[C@@H]4F)n3)n2nc1[C@H]1C[C@@H](O)C1]

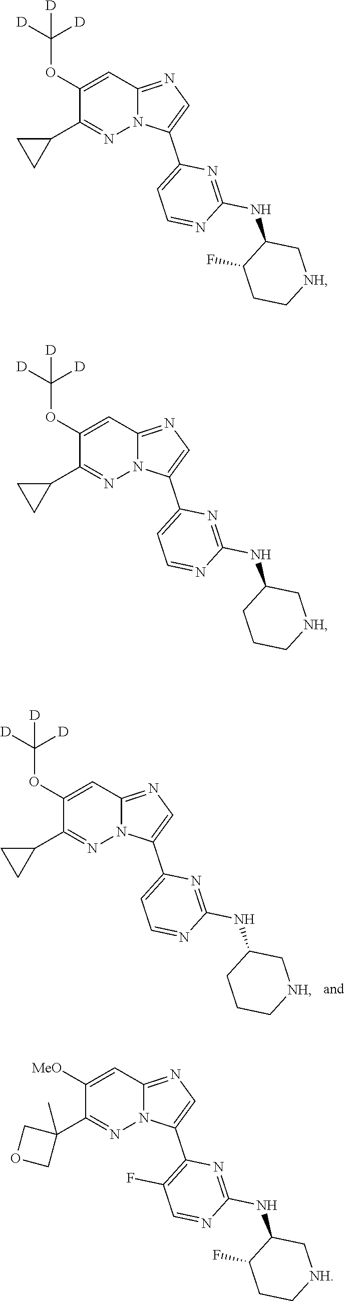 [SMILES: [2H]C([2H])([2H])Oc1cc2ncc(-c3ccnc(N[C@H]4CNCC[C@@H]4F)n3)n2nc1C1CC1 ; [2H]C([2H])([2H])Oc1cc2ncc(-c3ccnc(N[C@@H]4CCCNC4)n3)n2nc1C1CC1 ; [2H]C([2H])([2H])Oc1cc2ncc(-c3ccnc(N[C@H]4CCCNC4)n3)n2nc1C1CC1 ; COc1cc2ncc(-c3nc(N[C@H]4CNCC[C@@H]4F)ncc3F)n2nc1C1(C)COC1]

Clause 121. The compound of clause 101 or 102, wherein the compound of Formula (III) is a compound of Formula (IIIa-5010-WO50):

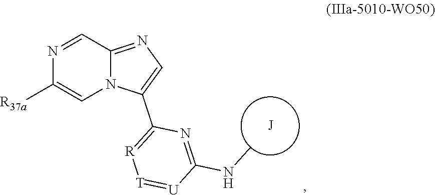

or a salt, ester, solvate, optical isomer, geometric isomer, or salt of an isomer thereof;
wherein: R is N or CR₃₁; T is N or CR₃₂; U is N or CR₃₃;

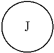

is

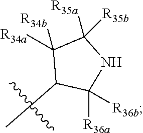

R₃₇ₐ is selected from halogen, C₁-C₆ alkyl, C₁-C₆ alkoxy, C₃-C₆ cycloalkyl, —O—(C₃-C₆ cycloalkyl), C₂-C₆ heterocyclyl, imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH, deuterium, and halogen, and wherein C₃-C₆ cycloalkyl, —O—(C₃-C₆ cycloalkyl), and C₂-C₆ heterocyclyl are each optionally substituted with one or more substituents selected from —OH, C₁-C₆ alkyl, and halogen; R³¹, R₃₂, and R₃₃ are each independently selected from H, C₁-C₆ alkoxy, and halogen; R₃₄ₐ, R_{34b}, R₃₅ₐ, R_{35b}, R₃₆ₐ, and R_{36b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R₃₈ₐ and R_{38b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of R, T, or U is N.

Clause 122. The compound of clause 121, wherein at least one of (i)-(iv) applies: (i) each of R_{34b}, R₃₅ₐ, R_{35b}, R₃₆ₐ, and R_{36b} is H and R₃₄ₐ is F, (ii) R₃₁, R₃₂, and R₃₃, if present, are H; (iii) R is CR₃₁ wherein R₃₁ is F, T is CR₃₂ wherein R₃₂ is H, and U is N; and (iv) R₃₇ₐ is selected from

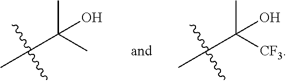

Clause 123. The compound of clause 121 or 122, with the proviso that R₃₇ₐ is not

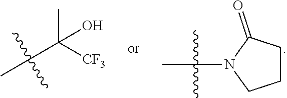

Clause 124. The compound of any one of clauses 121 to 123, wherein the compound of Formula (IIIa-5010-WO50) is:

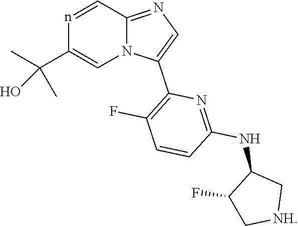

Clause 125. The compound of clause 101 or 102, wherein the compound of Formula (III) is a compound of Formula (IIIb-5010-WO50):

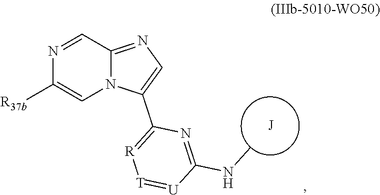

or a salt, ester, solvate, optical isomer, geometric isomer, or salt of an isomer thereof;
wherein: R is N or CR₃₁; T is N or CR₃₂; U is N or CR₃₃;

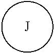

is

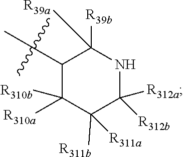

R_{37b} is selected from halogen, C₁-C₆ alkyl, C₁-C₆ alkoxy, C₃-C₆ cycloalkyl, —O—(C₃-C₆ cycloalkyl), C₂-C₆ heterocyclyl, imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH, deuterium, and halogen, and wherein C₃-C₆ cycloalkyl, —O—(C₃-C₆ cycloalkyl), and C₂-C₆ heterocyclyl are each optionally substituted with one or more substituents selected from —OH, C₁-C₆ alkyl, and halogen; R₃₁, R₃₂, and R₃₃ are each independently selected from H, C₁-C₆ alkoxy, and halogen; R₃₉ₐ, R_{39b}, R₃₁₀ₐ, R_{310b}, R₃₁₁ₐ, R_{311b}, R₃₁₂ₐ, and R_{312b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; R^{38a} and R_{38b} are each independently selected from H, C₁-C₆ alkyl, and C₃-C₆ cycloalkyl, wherein C₁-C₆ alkyl and C₃-C₆ cycloalkyl are each optionally substituted with one or more halogen; and one of R, T, or U is N.

Clause 126. The compound of clause 125, wherein at least one of (i)-(v) applies: (i) each of R₃₉ₐ, R_{39b}, R₃₁₀ₐ, R_{310b}, R₃₁₁ₐ, R_{311b}, R₃₁₂ₐ, and R_{312b} is H; (ii) each of R₃₉ₐ, R_{39b}, R_{310b}, R₃₁₁ₐ, R_{311b}, R₃₁₂ₐ, and R_{312b} is H and R₃₁₀ₐ is F; (iii) R₃₁, R₃₂, and R₃₃, if present, are H; (iv) R is CR₃₁ wherein R₃₁ is F, T is CR₃₂ wherein R₃₂ is H, and U is N; and (v) R_{37b} is selected from

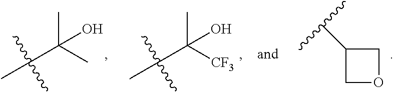

Clause 127. The compound of clause 125 or 126, with the proviso that: when R^{37b} is

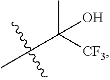 [SMILES: *C(C)(O)C(F)(F)F]

at least one of R₃₁, R₃₂, and R₃₃ is present and selected from C₁-C₆ alkoxy and halogen.

Clause 128. The compound of any one of clauses 125 to 127, wherein the compound of Formula (IIIb-5010-WO50) is selected from:

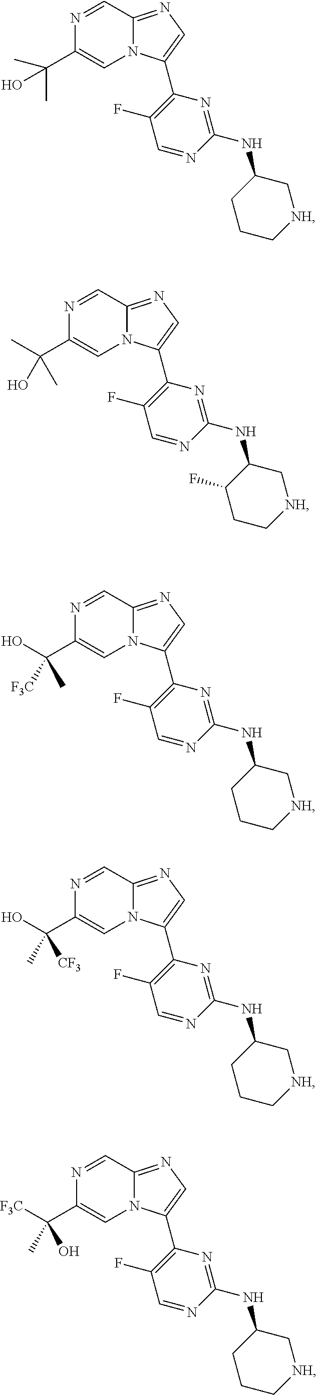 [SMILES: CC(C)(O)c1cn2c(-c3nc(N[C@@H]4CCCNC4)ncc3F)cnc2cn1 ; CC(C)(O)c1cn2c(-c3nc(N[C@H]4CNCC[C@@H]4F)ncc3F)cnc2cn1 ; C[C@@](O)(c1cn2c(-c3nc(N[C@@H]4CCCNC4)ncc3F)cnc2cn1)C(F)(F)F ; C[C@](O)(c1cn2c(-c3nc(N[C@@H]4CCCNC4)ncc3F)cnc2cn1)C(F)(F)F ; C[C@@](O)(c1cn2c(-c3nc(N[C@@H]4CCCNC4)ncc3F)cnc2cn1)C(F)(F)F]

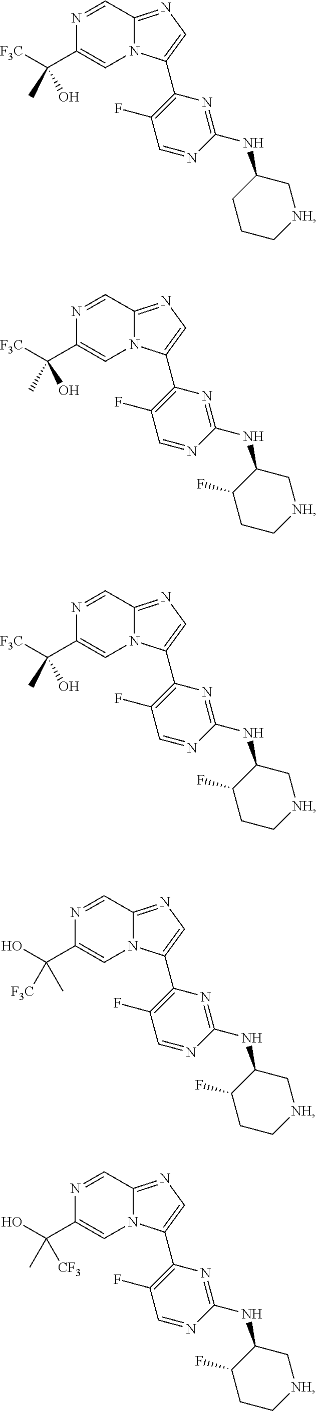 [SMILES: C[C@](O)(c1cn2c(-c3nc(N[C@@H]4CCCNC4)ncc3F)cnc2cn1)C(F)(F)F ; C[C@@](O)(c1cn2c(-c3nc(N[C@H]4CNCC[C@@H]4F)ncc3F)cnc2cn1)C(F)(F)F ; C[C@](O)(c1cn2c(-c3nc(N[C@H]4CNCC[C@@H]4F)ncc3F)cnc2cn1)C(F)(F)F ; CC(O)(c1cn2c(-c3nc(N[C@H]4CNCC[C@@H]4F)ncc3F)cnc2cn1)C(F)(F)F ; CC(O)(c1cn2c(-c3nc(N[C@H]4CNCC[C@@H]4F)ncc3F)cnc2cn1)C(F)(F)F]

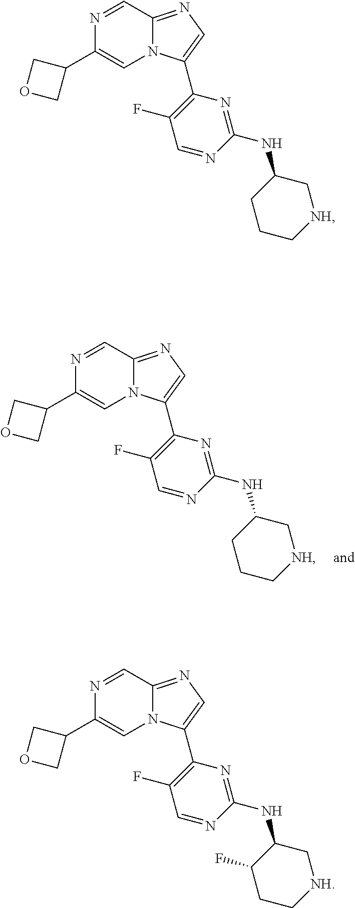 [SMILES: Fc1cnc(N[C@@H]2CCCNC2)nc1-c1cnc2cnc(C3COC3)cn12 ; Fc1cnc(N[C@H]2CCCNC2)nc1-c1cnc2cnc(C3COC3)cn12 ; Fc1cnc(N[C@H]2CNCC[C@@H]2F)nc1-c1cnc2cnc(C3COC3)cn12]

Clause 129. The compound of any one of clauses 101 to 128, wherein the compound is an inhibitor of at least one of IRAK1, IRAK4, and FLT3.

Clause 130. The compound of any one of clauses 101 to 129, wherein the compound is an inhibitor of at least two of IRAK1, IRAK4, and FLT3.

Clause 131. The compound of any one of clauses 101 to 130, wherein the compound is an inhibitor of IRAK1 and IRAK4 and not an inhibitor of FLT3.

Clause 132. The compound of any one of clauses 101 to 130, wherein the compound is an inhibitor of each of IRAK1, IRAK4, and FLT3.

Clause 133. The compound of any one of clauses 129, 130, or 131, wherein FLT3 is selected from WT FLT3, activated FLT3, and mutated FLT3.

Clause 134. The compound of clause 133, wherein the mutated FLT3 is D835Y mutated FLT3 or F691L mutated FLT3.

Clause 135. A composition comprising a compound of any one of clauses 101 to 134, wherein the composition further comprises a formulary ingredient, an adjuvant, or a carrier.

Clause 136. The composition of clause 135, wherein the composition is used in combination with one or more of: a chemotherapy agent, a BCL2 inhibitor, an immune modulator, a BTK inhibitor, a DNA methyltransferase inhibitor/hypomethylating agent, an anthracycline, a histone deacetylase (HDAC) inhibitor, a purine nucleoside analogue (antimetabolite), an isocitrate dehydrogenase 1 or 2 (IDH1 and/or IDH2) inhibitor, an antibody-drug conjugate, an mAbs/immunotherapy, a Plk inhibitor, a MEK inhibitor, a CDK inhibitor, a CDK9 inhibitor, a CDK8 inhibitor, a retinoic acid receptor agonist, a TP53 activator, a CELMoD, a smoothened receptor antagonist, an ERK inhibitor including an ERK2/MAPK1 or ERK1/MAPK3 inhibitor, a PI3K inhibitor, an mTOR inhibitor, a steroid or glucocorticoid, a steroid or glucocorticoid receptor modulator, an EZH2 inhibitor, a hedgehog (Hh) inhibitor, a Topoisomerase I inhibitor, a Topoisomerase II inhibitor, an aminopeptidase/Leukotriene A4 hydrolase inhibitor, a FLT3/Axl/ALK inhibitor, a FLT3/KIT/PDGFR, PKC, and/or KDR inhibitor, a Syk inhibitor, an E-selectin inhibitor, an NEDD8-activator, an MDM2 inhibitor, a PLK1 inhibitor, an Aura A inhibitor, an aurora kinase inhibitor, an EGFR inhibitor, an AuroraB/C/VEGFR1/2/3/FLT3/CSF-1R/Kit/PDGFRA/B inhibitor, an AKT 1, 2, and/or 3 inhibitor, a ABL1/2/SRC/EPHA2/LCK/YES1/KIT/PDGFRB/FYN inhibitor, a farnesyltransferase inhibitor, a BRAF/MAP2K1/MAP2K2 inhibitor, a Menin-KMT2A/MLL inhibitor, and a multikinase inhibitor.

Clause 137. The composition of clause 136, wherein the composition is used in combination with at least one of a BCL2 inhibitor, a BTK inhibitor, a glucocorticoid, a CDK inhibitor, and a DNA methyltransferase inhibitor.

Clause 138. The composition of clause 137 wherein the BCL2 inhibitor is venetoclax or a pharmaceutically acceptable salt thereof.

Clause 139. The composition of clause 137, wherein the BTK inhibitor is ibrutinib or a pharmaceutically acceptable salt thereof.

Clause 140. The composition of clause 137, wherein the glucocorticoid is selected from dexamethasone, methylprednisolone, prednisolone or a pharmaceutically acceptable salt of any one thereof.

Clause 141. The composition of clause 137, wherein the CDK inhibitor is a CDK4 inhibitor, a CDK6 inhibitor, a CDK7 inhibitor, and/or a CDK9 inhibitor.

Clause 142. The composition of clause 137, wherein the CDK inhibitor is selected from CDK4/6 inhibitor Palbociclib, CDK7 inhibitor THZ1, and/or CDK9 inhibitors BAY1251152 and Atuveciclib, or a pharmaceutically acceptable salt of any one thereof.

Clause 143. The composition of clause 137, wherein the DNA methyltransferase inhibitor is azacitidine or a pharmaceutically acceptable salt thereof.

Clause 144. A method of treating a disease or disorder in a subject, the method comprising administering to the subject a therapeutically effective amount of a compound of any one of clauses 101 to 134 or a composition of any one of clauses 135 to 143.

Clause 145. The method of clause 144, wherein the method comprises administering to the subject a composition comprising the therapeutically effective amount of the compound of clause 101 and a formulary ingredient, an adjuvant, or a carrier.

Clause 146. The method of clause 144 or 145, wherein the disease or disorder is responsive to at least one of interleukin-1 receptor-associated kinase (IRAK) inhibition and fins-like tyrosine kinase 3 (FLT3) inhibition.

Clause 147. The method of any one of clauses 144 to 146, wherein the administration comprises parenteral administration, a mucosal administration, intravenous administration, subcutaneous administration, topical administration, intradermal administration, oral administration, sublingual administration, intranasal administration, or intramuscular administration.

Clause 148. The method of any one of clauses 144 to 147, wherein the compound is administered to the subject in an amount of from about 0.005 mg/kg subject body weight to about 1,000 mg/kg subject body weight.

Clause 149. The method of any one of clauses 144 to 148, wherein the disease or disorder comprises a hematopoietic cancer.

Clause 150. The method of any one of clause 149, wherein the hematopoietic cancer is selected from myelodysplastic syndrome (MDS), acute myeloid leukemia (AML), lymphoma, leukemia, chronic lymphocytic leukemia (CLL), chronic myeloid leukemia (CML), acute lymphoblastic leukemia (ALL), non-Hodgkin lymphoma, Waldenstrom's macroglobulinemia, B cell lymphoma, diffuse large B-cell lymphoma (DLBCL), DLBCL with MYD88 mutation, follicular lymphoma, and marginal zone lymphoma.

Clause 151. The method of clause 150, wherein the hematopoietic cancer is selected from MDS and AML.

Clause 152. The method of any one of clauses 144 to 148, wherein the disease or disorder comprises at least one cancer selected from glioblastoma multiforme, endometrial cancer, melanoma, prostate cancer, lung cancer, breast cancer, kidney cancer, bladder cancer, basal cell carcinoma, thyroid cancer, squamous cell carcinoma, neuroblastoma, ovarian cancer, renal cell carcinoma, hepatocellular carcinoma, colon cancer, pancreatic cancer, rhabdomyosarcoma, meningioma, gastric cancer, Glioma, oral cancer, nasopharyngeal carcinoma, rectal cancer, stomach cancer, and uterine cancer, or combinations thereof.

Clause 153. The method of any one of clauses 144 to 148, wherein the disease or disorder comprises one or more inflammatory diseases or autoimmune disease selected from chronic inflammation, sepsis, rheumatoid arthritis, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, colitis, and atopic dermatitis.

Clause 154. The method of any one of clauses 44 to 48, wherein the disease or disorder comprises: (i) MDS, MDS with a splicing factor mutation, MDS with a mutation in isocitrate dehydrogenase 1, MDS with a mutation in isocitrate dehydrogenase 2; or (ii) AML with a splicing factor mutation, AML having enhanced IRAK4-Long expression and/or activity relative to IRAK4-Short, and/or wherein the AML is not driven by FLT3 mutations but expresses IRAK4-Long.

Clause 155. The method of clause 154, wherein the MDS with a splicing factor mutation comprises MDS with a splicing factor mutation in U2AF1 or SF3B1 and the AML splicing factor mutation comprises AML with a splicing factor mutation in U2AF1 or SF3B1.

Clause 156. The method of any one of clauses 144 to 148, wherein the disease or disorder comprises DLBCL, and wherein the DLBCL comprises a L265P MYD88 mutant (ABC) subtype of DLBCL or a S219C MYD88 mutant (GCB) subtype of DLBCL.

Clause 157. The method of any one of clauses 144 to 156, further comprising administering to the subject one or more additional therapies selected from: a chemotherapy agent, a BCL2 inhibitor, an immune modulator, a BTK inhibitor, a DNA methyltransferase inhibitor/hypomethylating agent, an anthracycline, a histone deacetylase (HDAC) inhibitor, a purine nucleoside analogue (antimetabolite), an isocitrate dehydrogenase 1 or 2 (IDH1 and/or IDH2) inhibitor, an antibody-drug conjugate, an mAbs/immunotherapy, a Plk inhibitor, a MEK inhibitor, a CDK inhibitor, a CDK9 inhibitor, a CDK8 inhibitor, a retinoic acid receptor agonist, a TP53 activator, a CELMoD, a smoothened receptor antagonist, an ERK inhibitor including an ERK2/MAPK1 or ERK1/MAPK3 inhibitor, a PI3K inhibitor, an mTOR inhibitor, a steroid or glucocorticoid, a steroid or glucocorticoid receptor modulator, an EZH2 inhibitor, a hedgehog (Hh) inhibitor, a Topoisomerase I inhibitor, a Topoisomerase II inhibitor, an aminopeptidase/Leukotriene A4 hydrolase inhibitor, a FLT3/Axl/ALK inhibitor, a FLT3/KIT/PDGFR, PKC, and/or KDR inhibitor, a Syk inhibitor, an E-selectin inhibitor, an NEDD8-activator, an MDM2 inhibitor, a PLK1 inhibitor, an Aura A inhibitor, an aurora kinase inhibitor, an EGFR inhibitor, an AuroraB/C/VEGFR1/2/3/FLT3/CSF-1R/Kit/PDGFRA/B inhibitor, an AKT 1, 2, and/or 3 inhibitor, a ABL1/2/SRC/EPHA2/LCK/YES1/KIT/PDGFRB/FYN inhibitor, a farnesyltransferase inhibitor, a BRAF/MAP2K1/MAP2K2 inhibitor, a Menin-KMT2A/MILL inhibitor, and a multikinase inhibitor.

Clause 158. The method of any one of clauses 144 to 157, wherein the disease or disorder is responsive to at least one of BCL2 inhibition, BTK inhibition, CDK inhibition, and DNA methyltransferase inhibition; or wherein the disease or disorder is sensitive to anti-inflammatory glucocorticoids.

Clause 159. The method of clause 158, wherein the additional therapy is at least one of a BCL2 inhibitor, a BTK inhibitor, a glucocorticoid, a CDK inhibitor, and a DNA methyltransferase inhibitor.

Clause 160. The method of clause 159, wherein the BCL2 inhibitor is venetoclax or a pharmaceutically acceptable salt thereof.

Clause 161. The method of any one of clauses 144 to 159, wherein the disease or disorder is a BCL2 inhibitor resistant disease or disorder.

Clause 162. The method of any one of clauses 144 to 159, wherein the disease or disorder is a venetoclax resistant disease or disorder.

Clause 163. The method of any one of clauses 144 to 159, wherein the disease or disorder is BCL2 inhibitor resistant acute myeloid leukemia (AML).

Clause 164. The method of any one of clauses 144 to 159, wherein the disease or disorder is venetoclax resistant acute myeloid leukemia (AML)

Clause 165. The method of any one of clauses 144 to 159, wherein the disease or disorder is BCL2 inhibitor resistant refractory acute myeloid leukemia (AML).

Clause 166. The method of any one of clauses 144 to 159, wherein the disease or disorder is venetoclax resistant refractory acute myeloid leukemia (AML).

Clause 167. The method of any one of clauses 144 to 159, wherein the disease or disorder is BCL2 inhibitor resistant relapsed acute myeloid leukemia (AML).

Clause 168. The method of any one of clauses 144 to 159, wherein the disease or disorder is venetoclax resistant relapsed acute myeloid leukemia (AML).

Clause 169. The method of clause 159, wherein the BTK inhibitor is ibrutinib or a pharmaceutically acceptable salt thereof.

Clause 170. The method of any one of clauses 144 to 159, wherein the disease or disorder is a BTK inhibitor resistant disease or disorder.

Clause 171. The method of any one of clauses 144 to 159, wherein the disease or disorder is an ibrutinib resistant disease or disorder.

Clause 172. The method of clause 159, wherein the glucocorticoid is selected from dexamethasone, methylprednisolone, prednisolone, or a pharmaceutically acceptable salt of any one thereof.

Clause 173. The method of any one of clauses 144 to 159, wherein the disease or disorder is sensitive to anti-inflammatory glucocorticoids.

Clause 174. The method of any one of clauses 144 to 159, wherein the disease or disorder is a dexamethasone, methylprednisolone, or prednisolone resistant disease or disorder.

Clause 175. The method of clause 159, wherein the CDK inhibitor is selected from CDK4/6 inhibitor palbociclib, CDK7 inhibitor THZ1, and/or CDK9 inhibitors BAY1251152 and atuveciclib, or a pharmaceutically acceptable salt of any one thereof.

Clause 176. The method of any one of clauses 144 to 159, wherein the disease or disorder is a CDK inhibitor resistant disease or disorder.

Clause 177. The method of any one of clauses 144 to 159, wherein the disease or disorder is a palbociclib, THZ1, BAY 12511152, or atuveciclib resistant disease or disorder.

Clause 178. The method of clause 159, wherein the DNA methyltransferase inhibitor is azacitidine or a pharmaceutically acceptable salt thereof.

Clause 179. The method of any one of clauses 144 to 159, wherein the disease or disorder is a DNA methyltransferase inhibitor resistant disease or disorder.

Clause 180. The method of any one of clauses 144 to 159, wherein the disease or disorder is an azacitidine resistant disease or disorder.

Clause 181. The method of any one of clauses 144 to 159, wherein the disease or disorder is a BCL2 inhibitor and DNA methyltransferase inhibitor resistant disease or disorder.

Clause 182. The method of any one of clauses 144 to 159, wherein the disease or disorder is a venetoclax and azacitidine resistant disease or disorder.

Clause 183. The method of clause 159, wherein the BCL2 inhibitor is venetoclax or a pharmaceutically acceptable salt thereof and the DNA methyltransferase inhibitor is azacitidine or a pharmaceutically acceptable salt thereof.

Clause 184. The method of any one of clauses 144 to 159, wherein the disease or disorder is a FLT3 inhibitor resistant disease or disorder.

Clause 185. The method of any one of clauses 144 to 159, wherein the disease or disorder is FLT3 inhibitor resistant acute myeloid leukemia (AML).

Clause 186. The method of any one of clauses 144 to 159, wherein the disease or disorder is FLT3 inhibitor resistant refractory acute myeloid leukemia (AML)

Clause 187. The method of any one of clauses 144 to 159, wherein the disease or disorder is FLT3 inhibitor resistant relapsed acute myeloid leukemia (AML).

Clause 188. The method of clause 157, wherein the compound of any one of clauses 101 to 134 or the composition of any one of clauses 135 to 143 and the one or more additional therapies are administered together in one administration or composition.

Clause 189. The method of clause 157, wherein the compound of any one of clauses 101 to 134 or the composition of any one of clauses 135 to 143 and the one or more additional therapies are administered separately in more than one administration or more than one composition.

Clause 190. The method of any one of clauses 144 to 189, wherein the disease or disorder is alleviated by inhibiting at least one of IRAK1, IRAK4, and FLT3 in the subject.

Clause 191. The method of any one of clauses 144 to 190, wherein the disease or disorder is alleviated by inhibiting at least two of IRAK1, IRAK4, and FLT3 in the subject.

Clause 192. The method of any one of clauses 144 to 191, wherein the disease or disorder is alleviated by inhibiting IRAK1 and IRAK4 in the subject.

93. The method of any one of clauses 144 to 191, wherein the disease or disorder is alleviated by inhibiting IRAK1, IRAK4, and FLT3 in the subject.

Clause 194. The method of any one of clauses 184, 185, or 187, wherein FLT3 is selected from WT FLT3, activated FLT3, and mutated FLT3.

Clause 195. The method of clause 194, wherein the mutated FLT3 is D835Y mutated FLT3 or F691L mutated FLT3.

Clause 196. The method of any one of clauses 144 to 195, wherein the compound or composition inhibits at least one of IRAK1, IRAK4, and FLT3 in the subject.

Clause 197. The method of any one of clauses 144 to 196, wherein the compound or composition inhibits at least two of IRAK1, IRAK4, and FLT3 in the subject.

Clause 198. The method of any one of clauses 144 to 197, wherein the compound or composition inhibits IRAK1 and IRAK4 in the subject and does not inhibit FLT3 in the subject.

Clause 199. The method of any one of clauses 144 to 197, wherein the compound inhibits IRAK1, IRAK4, and FLT3 in the subject.

Clause 200. The method of any one of clauses 196, 197, or 199, wherein FLT3 is selected from WT FLT3, activated FLT3, and mutated FLT3.

Clause 201. The method of clause 200, wherein the mutated FLT3 is D835Y mutated FLT3 or F691L mutated FLT3.

Clause 202. The method of any one of clauses 144 to 200, wherein the compound is a compound of any one of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof.

Clause 203. A method of increasing survivability in a subject diagnosed with acute myeloid leukemia (AML) or suspected of having AML, the method comprising administering to the subject a therapeutically effective amount of a compound of any one of clauses 101 to 134 or the composition of any one of clauses 135 to 143.

Clause 204. The method of clause 203, wherein the survivability of the subject is increased compared to a subject treated with a therapeutically effective amount of the standard of care for AML.

Clause 205. The method of clause 204, wherein the standard of care for AML comprises gilteritinib or a pharmaceutically acceptable salt thereof.

Clause 206. The method of any one of clauses 203 to 205, wherein the subject is a human.

Clause 207. The method of clause 206, wherein the survivability of the subject is increased by about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, or about 20 years compared to a subject treated with a therapeutically effective amount of the standard of care for AML.

Clause 208. The method of any one of clauses 203 to 207, comprising administering to the subject the therapeutically effective amount of a compound of any one of clauses 101 to 134 or the composition of any one of clauses 135 to 143 about every 6 hours, every 12 hours, every 18 hours, once a day, every other day, every 3 days, every 4 days, every 5 days, every 6 days, or once a week.

Clause 209. The method of any one of clauses 203 to 208, further comprising administering to the subject one or more additional therapies selected from: a chemotherapy agent, a BCL2 inhibitor, an immune modulator, a BTK inhibitor, a DNA methyltransferase inhibitor/hypomethylating agent, an anthracycline, a histone deacetylase (HDAC) inhibitor, a purine nucleoside analogue (antimetabolite), an isocitrate dehydrogenase 1 or 2 (IDH1 and/or IDH2) inhibitor, an antibody-drug conjugate, an mAbs/immunotherapy, a Plk inhibitor, a MEK inhibitor, a CDK inhibitor, a CDK9 inhibitor, a CDK8 inhibitor, a retinoic acid receptor agonist, a TP53 activator, a CELMoD, a smoothened receptor antagonist, an ERK inhibitor including an ERK2/MAPK1 or ERK1/MAPK3 inhibitor, a PI3K inhibitor, an mTOR inhibitor, a steroid or glucocorticoid, a steroid or glucocorticoid receptor modulator, an EZH2 inhibitor, a hedgehog (Hh) inhibitor, a Topoisomerase I inhibitor, a Topoisomerase II inhibitor, an aminopeptidase/Leukotriene A4 hydrolase inhibitor, a FLT3/Axl/ALK inhibitor, a FLT3/KIT/PDGFR, PKC, and/or KDR inhibitor, a Syk inhibitor, an E-selectin inhibitor, an NEDD8-activator, an MDM2 inhibitor, a PLK1 inhibitor, an Aura A inhibitor, an aurora kinase inhibitor, an EGFR inhibitor, an AuroraB/C/VEGFR1/2/3/FLT3/CSF-1R/Kit/PDGFRA/B inhibitor, an AKT 1, 2, and/or 3 inhibitor, a ABL1/2/SRC/EPHA2/LCK/YES1/KIT/PDGFRB/FYN inhibitor, a farnesyltransferase inhibitor, a BRAF/MAP2K1/MAP2K2 inhibitor, a Menin-KMT2A/MLL inhibitor, and a multikinase inhibitor.

Clause 210. The method of clause 209, wherein the additional therapy is at least one of a BCL2 inhibitor, a BTK inhibitor, a glucocorticoid, a CDK inhibitor, and a DNA methyltransferase inhibitor.

Clause 211. The method of clause 210, wherein the BCL2 inhibitor is venetoclax or a pharmaceutically acceptable salt thereof, the BTK inhibitor is ibrutinib or a pharmaceutically acceptable salt thereof, the glucocorticoid is selected from dexamethasone, methylprednisolone, prednisolone, or a pharmaceutically acceptable salt of any one thereof, the CDK inhibitor is selected from CDK4/6 inhibitor palbociclib, CDK7 inhibitor THZ1, and/or CDK9 inhibitors BAY1251152 and atuveciclib, or a pharmaceutically acceptable salt of any one thereof, and the DNA methyltransferase inhibitor is azacitidine or a pharmaceutically acceptable salt thereof.

Clause 212. The method of any one of clauses 203 to 211, wherein the AML is selected from: BCL2 inhibitor resistant AIL, BCL2 inhibitor resistant refractory AML, BCL2 inhibitor resistant relapsed AML, FLT3 inhibitor resistant AML, FLT3 inhibitor resistant refractory AMIL, or FLT3 inhibitor resistant relapsed AML.

Clause 213. The method of clause 209, wherein the compound of any one of clauses 101 to 134 or the composition of any one of clauses 135 to 143 and the one or more additional therapies are administered together in one administration or composition.

Clause 214. The method of clause 209, wherein the compound of any one of clauses 101 to 134 or the composition of any one of clauses 135 to 143 and the one or more additional therapies are administered separately in more than one administration or more than one composition.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### List of Abbreviations

In the accompanying procedures and schemes, abbreviations are used with the following meanings unless otherwise indicated: Ac=acetate; aq, aq.=aqueous; Ar=aryl; BOC, Boc=t-butyloxycarbonyl; Bn=benzyl; BSA=bovine serum albumin; Bu=butyl, t-Bu=tert-butyl; BuLi, n-BuLi=n-butyllithium; CBZ, Cbz=Benzyloxycarbonyl; conc, conc.=concentrated; c-Bu=cyclobutyl; c-Pr=cyclopropyl; Cy=cyclohexyl; DAST=(diethylamino)sulfur trifluoride; dba=dibenzylideneacetone; DCM=dichloromethane; DIAD=diisopropylazodicarboxylate; DIBAL, DIBAL-H=diisobutylaluminum hydride; DIEA=diisopropylethylamine; DMAC, DMA=dimethylacetamide; DME=1,2-dimethoxyethane; DMEM=Dulbecco's modified eagle medium; DMAP=4-dimethylaminopyridine; DMF=N,N-dimethylformamide; DMSO=dimethylsulfoxide; eq.=equivalent(s); EDC=N-[3-(dimethylamino)propyl]-N-ethylcarbodiimide; EDTA=ethylenediaminetetraacetic acid; ESI=electrospray ionization; Et=ethyl; EtOAc=ethyl acetate; EtOH=ethanol; FBS=Fetal Bovine Serum; h, hr=hour; HATU=N-[(dimethylamino)-1H-1,2,3-triazolo[4,5-b]pyridin-1-ylmethylene]-N-methylmethanaminium hexafluorophosphate N-oxide; HOAc=acetic acid; HOAt=3H-[1,2,3]-triazolo[4,5-b]pyridin-3-ol; HOBt=1H-benzotriazol-1-ol; HPLC=High pressure liquid chromatography; HTRF=homogenous time resolved fluorescence; IPA, i-PrOH=isopropanol; iPr=isopropyl; LAH=lithium aluminum hydride; LCMS=liquid chromatography-mass spectroscopy; LHMDS=lithium bis(trimethylsilyl)amide; Me=methyl; MeOH=methanol; min, min.=minute; μW=microwave; NaHMDS=sodium bis(trimethylsilyl)amide; NIS=1-iodoopyrrolidine-2,5-dione; NBS=1-bromopyrrolidine-2,5-dione; NCS=1-chloropyrrolidine-2,5-dione; NMP=N-methylpyrrolidinone; NMR=nuclear magnetic resonance; OMs, mesyl=methanesulfonyl; Oxone, OXONE=potassium peroxymonosulfate; PBS=phosphate buffered saline; Pd₂dba₃=tris(dibenzylidineacetone)dipalladium; Pd(dppf)Cl₂=[1,1′-bis(diphenylphosphino)ferrocene]dichloropalladium(II); Pd/C=palladium on activated carbon; Ph=phenyl; PMB=4-methoxybenzyl; PMBCI=1-(chloromethyl)-4-methoxybenzene; Pr=propyl; Py=pyridyl; QPhos=(1,2,3,4,5-pentaphenyl-1′-(di-tert-butylphosphino)ferrocene; RT, rt=room temperature; RuPhos Pd G3=(2-dicyclohexylphosphino-2′,6′-diisopropoxy-1,1′-biphenyl)[2-(2′-amino-1,1′-biphenyl)]palladium(II)methanesulfonate; sat.=saturated; TBAF=tetrabutylammonium fluoride; TBAI=tetrabutylammonium iodide; t-Bu=tert-butyl; TFA=trifluoroacetic acid; THE=tetrahydrofuran; TLC=thin layer chromatography; prep TLC=preparative thin layer chromatography; Tosyl=toluenesulfonyl; triflate, OTf=trifluoromethanesulfonate; triflic=trifluoromethanesulfonic; Xantphos=4,5-bis(diphenylphosphino)-9,9-dimethylxanthene; XPhos Pd G2 or XPhos-PD-G2=chloro(2-dicyclohexylphosphino-2′,4′,6′-triisopropyl-1,1′-biphenyl)[2-(2′-amino-1,1′-biphenyl)]palladium(II).

### General Methods

Unless otherwise stated, all reactions were carried out under an atmosphere of dry nitrogen in dried glassware. Indicated reaction temperatures refer to those of the reaction bath, while room temperature (rt) is noted as 25° C. Unless otherwise noted, all solvents were of anhydrous quality purchased from Aldrich Chemical Co. and were used as received. Commercially available starting materials and reagents were purchased from commercial suppliers and were used as received.

Analytical thin layer chromatography (TLC) was performed with Sigma Aldrich TLC plates (5×20 cm, 60 Å, 250 μm). Visualization was accomplished by irradiation under a 254 nm UV lamp. Chromatography on silica gel was performed using forced flow (liquid) of the indicated solvent system on Biotage KP-Sil pre-packed cartridges and using the Biotage SP-1 automated chromatography system. ¹H NMR spectra were recorded on a Varian Inova 400 MHz spectrometer. Chemical shifts are reported in ppm with the solvent resonance as the internal standard (DMSO-d6 2.50 ppm for ¹H). Data are reported as follows: chemical shift, multiplicity (s=singlet, d=doublet, t=triplet, q=quartet, quint=quintet, br=broad, m=multiplet), coupling constants, and number of protons. Low resolution mass spectra (electrospray ionization) were acquired on an Agilent Technologies 6130 quadrupole spectrometer coupled to the HPLC system. Unless otherwise noted, all LCMS ions listed are [M+H]⁺. If needed, products were purified via semi-preparative HPLC using the columns and mobile phases noted. Samples were analyzed for purity on an Agilent 1200 series LC/MS equipped with a Luna® C18 reverse phase (3 micron, 3×75 mm) column having a flow rate of 0.8-1.0 mL/min over a 7 minute gradient and an 8.5 minute run time (Method 1). Unless otherwise noted, the mobile phase was a mixture of acetonitrile (0.025% TFA) and H₂O (0.05% TFA), with temperature maintained at 50° C. Purity of final compounds was determined to be >95% using a 3 μL injection with quantitation by AUC at 220 and 254 nm (Agilent Diode Array Detector).

### Example 1

### Exemplary Synthetic Procedure #1 (Intermediates A-P)

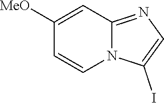 [SMILES: COc1ccn2c(I)cnc2c1]

### Intermediate A, 3-iodo-7-methoxyimidazo[1,2-a]pyridine

### Step A. 3-iodo-7-methoxyimidazo[1,2-a]pyridine

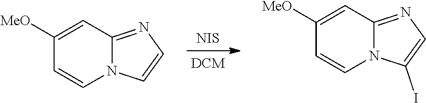

To a cooled 0° C. solution of 7-methoxyimidazo[1,2-a]pyridine (0.500 g, 3.37 mmol) in dichloromethane (5.0 mL) was added 1-iodopyrrolidine-2,5-dione (0.911 g, 4.05 mmol). The resulting mixture was then stirred for 3 hours while slowly warming to room temperature. The mixture was then concentrated under reduced pressure to give a crude product that was washed with ethyl acetate (3×5 mL) and filtered. The resulting solid was collected and dried under reduced pressure to provide the title compound: LCMS m/z 275.0 [M+H]⁺; ¹H NMR (400 MHz, CD₃OD) δ 8.15 (d, J=7.5 Hz, 1H), 7.49 (s, 1H), 6.94 (d, J=2.4 Hz, 1H), 6.79 (dd, J=2.5, 7.5 Hz, 1H), 3.92 (s, 3H).

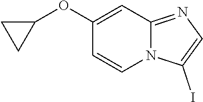 [SMILES: Ic1cnc2cc(OC3CC3)ccn12]

### Intermediate B, 7-cyclopropoxyimidazo[1,2-a]pyridine

### Step A. 6-chloro-7-fluoroimidazo[1,2-a]pyridine

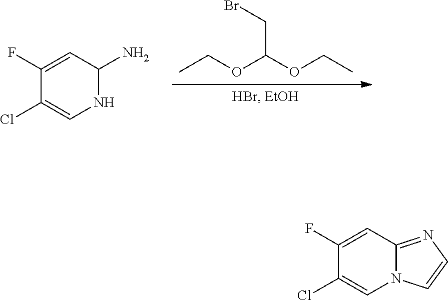

To a solution of 5-chloro-4-fluoro-pyridin-2-amine (2.00 g, 13.7 mmol) in ethanol (20 mL) were added 2-bromo-1,1-diethoxy-ethane (6.19 g, 31.4 mmol, 4.72 mL) and a solution of hydrogen bromide in acetic acid (33% v/v, 4.14 g, 17.1 mmol, 2.78 mL). The resulting reaction was stirred at 80° C. for 15 hours. The reaction was then cooled to room temperature, poured into saturated aqueous sodium bicarbonate solution (20 mL), and extracted with ethyl acetate (3×20 mL). The combined organic extracts were washed with saturated aqueous sodium chloride solution (20 mL), dried over sodium sulfate, filtered, and concentrated under reduced pressure. The resulting crude product was purified by flash chromatography on silica gel (0-60% ethyl acetate in petroleum ether) to provide the title compound: LCMS m/z 171.1 [M+H]⁺; ¹H NMR (400 MHz, CD₃OD) δ 8.65-8.85 (m, 1H), 7.83 (br d, J=6.2 Hz, 1H), 7.58 (dd, J=5.5, 1.4 Hz, 1H), 7.41 (br t, J=10.1 Hz, 1H).

### Step B. 6-chloro-7-cyclopropoxyimidazo[1,2-a]pyridine

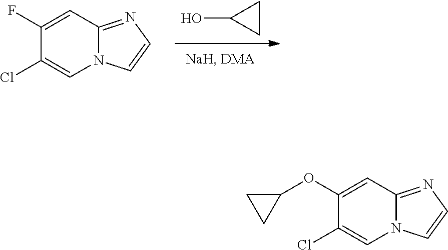

Sodium hydride (0.211 g, 5.28 mmol, 60% purity) was added in portions to a cooled 0° C. solution of cyclopropanol (0.306 g, 5.28 mmol) in N,N-dimethylacetamide (10 mL). The resulting reaction mixture was then stirred for 30 minutes while warming to room temperature. 6-Chloro-7-fluoroimidazo[1,2-a]pyridine (0.300 g, 1.76 mmol) was then added, and the mixture was stirred for an additional 16 hours under nitrogen atmosphere. The reaction mixture was then cooled to 0° C., quenched by addition of water (10 mL), and extracted with ethyl acetate (3×10 mL). The combined organic extracts were washed with saturated aqueous sodium chloride solution (10 mL), dried over sodium sulfate, filtered, and concentrated under reduced pressure. The resulting crude product was purified by flash chromatography on silica gel (0-100% ethyl acetate in petroleum ether) to provide the title compound: LCMS m/z 209.1 [M+H]⁺.

### Step C. 7-cyclopropoxyimidazo[1,2-a]pyridine

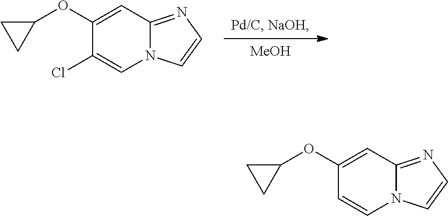 [SMILES: "MeOH" ; Clc1cn2ccnc2cc1OC1CC1 ; "Pd/C, NaOH," ; c1cn2ccc(OC3CC3)cc2n1]

A mixture of 6-chloro-7-(cyclopropoxy)imidazo[1,2-a]pyridine (0.110 g, 0.527 mmol), sodium hydroxide (0.063 g, 1.58 mmol), and 10% palladium on carbon (0.124 g, 0.105 mmol) in methanol (5 mL) was purged with hydrogen, and was then stirred at room temperature for 16 hours under hydrogen atmosphere. The mixture was then filtered and concentrated under reduced pressure. The resulting crude product was purified by flash chromatography on silica gel (0-100% ethyl acetate in petroleum ether) to provide the title compound: LCMS m/z 175.3 [M+H]⁺; ¹H NMR (400 MHz, CD₃OD) δ 8.33 (d, J=7.5 Hz, 1H), 7.72 (s, 1H), 7.45-7.53 (m, 1H), 7.21 (d, J=2.1 Hz, 1H), 6.69 (dd, J=7.4, 2.3 Hz, 1H), 3.86-4.00 (m, 1H), 0.90-0.96 (m, 2H), 0.77-0.85 (m, 2H).

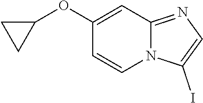 [SMILES: Ic1cnc2cc(OC3CC3)ccn12]

### Intermediate C, 7-cyclopropoxy-3-iodoimidazo[1,2-a]pyridine

### Step A. 7-cyclopropoxy-3-iodoimidazo[1,2-a]pyridine

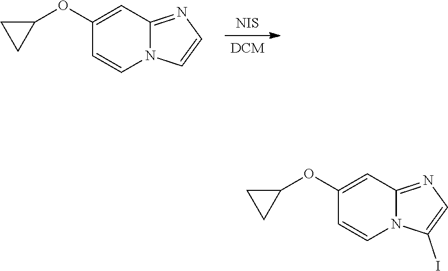

To a cooled 0° C. solution of 7-cyclopropoxyimidazo[1,2-a]pyridine (0.180 g, 1.03 mmol) in acetonitrile (5 mL) was added 1-iodopyrrolidine-2,5-dione (0.232 g, 1.03 mmol). The resulting mixture was stirred for 2 hours while slowly warming to room temperature. The reaction mixture was then filtered and concentrated under reduced pressure. The resulting crude product was purified by prep-TLC (dichloromethane: methanol=10:1) to provide the title compound: LCMS m/z 301.0 [M+H]⁺; ¹H NMR (400 MHz, CD₃OD) δ 8.22-8.19 (s, 1H), 7.58 (s, 1H), 7.24 (d, J=2.1 Hz, 1H), 6.85-6.82 (m, 1H), 4.12-4.09 (m, 1H), 0.95-0.89 (m, 2H), 0.84-0.78 (m, 2H).

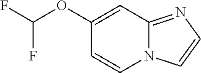 [SMILES: FC(F)Oc1ccn2ccnc2c1]

### Intermediate D, 7-(difluoromethoxy)imidazo[1,2-a]pyridine

### Step A. imidazo[1,2-a]pyridin-7-ol

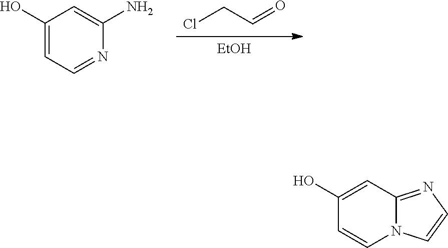

A mixture of 2-aminopyridin-4-ol (6.00 g, 54.5 mmol) and 2-chloroacetaldehyde (12.83 g, 163.5 mmol, 10.52 mL) in ethanol (40 mL) was stirred at 100° C. for 16 hours. The reaction mixture was then cooled to room temperature, filtered, and concentrated under reduced pressure. The resulting crude product was purified by HPLC (Phenomenex Luna C18 column, 15 micron, 250×70 mm; 0-10% acetonitrile in water containing 0.05% hydrochloric acid) to provide the title compound: LCMS m/z 135.1 [M+H]⁺; ¹H NMR (400 MHz, DMSO-d₆) δ 8.67 (d, J=7.4 Hz, 1H), 8.06 (d, J=2.1 Hz, 1H), 7.89 (d, J=2.3 Hz, 1H), 6.98-7.26 (m, 2H).

### Step B. 7-(difluoromethoxy)imidazo[1,2-a]pyridine

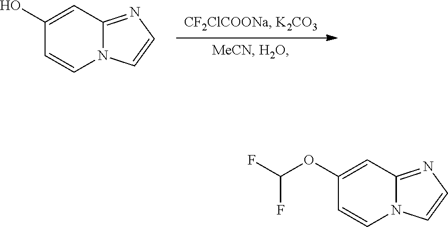

A mixture of imidazo[1,2-a]pyridin-7-ol (1.50 g, 11.2 mmol), sodium 2-chloro-2,2-difluoroacetate (8.52 g, 55.9 mmol), and potassium carbonate (3.09 g, 22.37 mmol) in water (10 mL) and acetonitrile (50 mL) was purged with nitrogen, and was then stirred at 110° C. for 16 hours under nitrogen atmosphere. The reaction was then cooled to room temperature, filtered, and concentrated under reduced pressure. The resulting crude product was purified by flash chromatography (Agela C18 column, 0-30% methanol in aqueous 10 mM NaHCO₃) to provide the title compound: LCMS m/z 185.1 [M+H]⁺; ¹H NMR (400 MHz, DMSO-d₆) δ 8.61 (d, J 7.4 Hz, 1H), 7.93 (s, 1H), 7.54-7.60 (m, 1H), 7.20-7.44 (m, 2H), 6.84 (dd, J=7.4, 2.4 Hz, 1H).

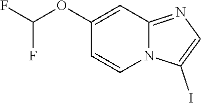 [SMILES: FC(F)Oc1ccn2c(I)cnc2c1]

### Intermediate E, 7-(difluoromethoxy)-3-iodoimidazo[1,2-a]pyridine

### Step A. 7-(difluoromethoxy)-3-iodoimidazo[1,2-a]pyridine

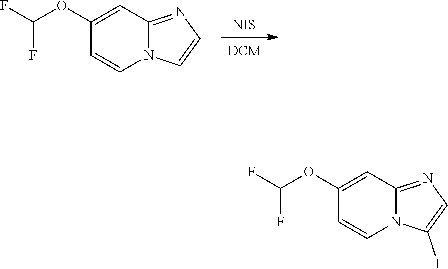

To a cooled 0° C. solution of 7-(difluoromethoxy)imidazo[1,2-a]pyridine (0.300 g, 1.63 mmol) in acetonitrile (8 mL) was added 1-iodopyrrolidine-2,5-dione (0.440 g, 1.96 mmol). The resulting mixture was stirred for 1 hour while slowly warming to room temperature, and was then concentrated under reduced pressure. The resulting crude product was purified by flash chromatography on silica gel (0-70% ethyl acetate in petroleum ether) to provide the title compound: LCMS m/z 310.8 [M+H]⁺; ¹H NMR (400 MHz, CD₃OD) δ 8.94 (d, J=7.5 Hz, 1H), 8.25 (s, 1H), 7.87 (d, J=3.5 Hz, 1H), 7.72-7.43 (m, 2H).

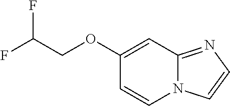 [SMILES: FC(F)COc1ccn2ccnc2c1]

### Intermediate F, 7-(2,2-difluoroethoxy)imidazo[1,2-a]pyridine

### Step A. 7-(2,2-difluoroethoxy)imidazo[1,2-a]pyridine

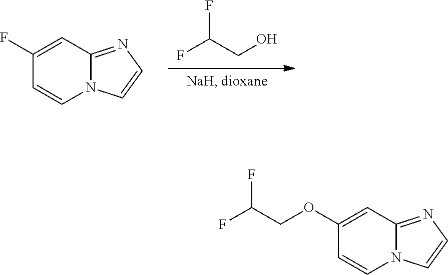

To a cooled 0° C. solution of 2,2-difluoroethanol (9.04 g, 110 mmol) in dioxane (50 mL) was added sodium hydride (4.41 g, 110 mmol, 60% purity). The resulting mixture was stirred at 0° C. for 30 minutes. 7-Fluoroimidazo[1,2-a]pyridine (3.00 g, 22.0 mmol) was then added, and the resulting mixture was warmed to 80° C. and stirred for 10 hours. The reaction mixture was then cooled to room temperature, quenched by addition of ice water (200 mL), and extracted with ethyl acetate (4×200 mL). The organic extracts were combined, washed with saturated aqueous sodium chloride solution (200 mL), dried over sodium sulfate, filtered, and concentrated under reduced pressure. The resulting crude product was purified by flash chromatography on silica gel (0-100% ethyl acetate in petroleum ether) to provide the title compound: LCMS m/z 198.9 [M+H]⁺; ¹H NMR (400 MHz, CD₃OD) δ 8.31 (d, J=7.5 Hz, 1H), 7.69 (s, 1H), 7.43 (d, J=1.4 Hz, 1H), 6.94 (d, J=2.4 Hz, 1H), 6.70 (dd, J=2.5, 7.5 Hz, 1H), 6.40-6.07 (m, 1H), 4.33 (dt, J=3.7, 13.7 Hz, 2H).

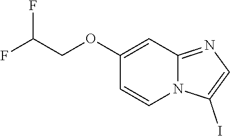 [SMILES: FC(F)COc1ccn2c(I)cnc2c1]

### Intermediate G, 7-(2, 2-difluoroethoxy)-3-iodoimidazo[1,2-a]pyridine

### Step A. 7-(2, 2-difluoroethoxy)-3-iodoimidazo[1,2-a]pyridine

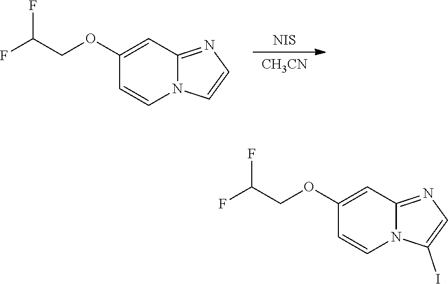

To a cooled 0° C. solution of 7-(2, 2-difluoroethoxy)imidazo[1,2-a]pyridine (0.500 g, 2.52 mmol) in acetonitrile (10 mL) was added 1-iodopyrrolidine-2,5-dione (0.568 g, 2.52 mmol). The resulting mixture was stirred for 5 hours while slowly warming to room temperature, and was then concentrated under reduced pressure. The resulting crude product was purified by flash chromatography on silica gel (0-75% ethyl acetate in petroleum ether) to provide the title compound: LCMS m/z 324.8 [M+H]⁺.

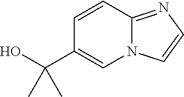

### Intermediate H, 2-(imidazo[1,2-a]pyridin-6-yl)propan-2-ol

### Step A. 2-(imidazo[1,2-a]pyridin-6-yl)propan-2-ol

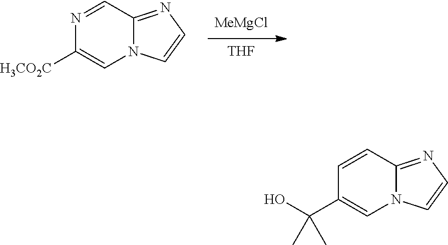

To a cooled 0° C. solution of methyl imidazo[1,2-a]pyridine-6-carboxylate (1.0 g, 5.7 mmol) in tetrahydrofuran (20 mL) was added a solution of methyl magnesium bromide in diethyl ether (3.0 M, 7.57 mL, 22.7 mmol). The resulting reaction mixture was stirred for 2 hours while warming to room temperature. The reaction was then cooled to 0° C., quenched by addition of water (20 mL), and extracted with ethyl acetate (2×15 mL). The combined organic extracts were washed with saturated aqueous sodium chloride solution (2×15 mL), dried over sodium sulfate, filtered, and concentrated under reduced pressure to provide the title compound: LCMS m/z 177.2 [M+H]⁺; ¹H NMR (400 MHz, CD₃OD) δ 8.51 (s, 1H), 7.83 (s, 1H), 7.55 (d, J=1.1 Hz, 1H), 7.53-7.49 (m, 1H), 7.48-7.43 (m, 1H), 1.64-1.57 (m, 6H).

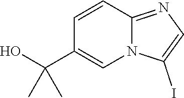

### Intermediate I, 2-(3-iodoimidazo[1,2-a]pyridin-6-yl)propan-2-ol

### Step A. 2-(3-iodoimidazo[1,2-a]pyridin-6-yl)propan-2-ol

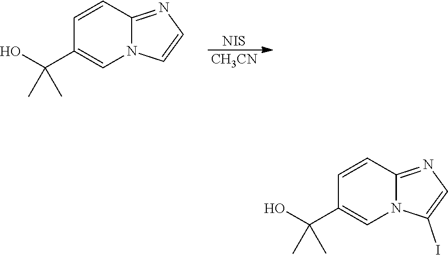

To a cooled 0° C. solution of 2-imidazo[1,2-a]pyridin-6-ylpropan-2-ol (1.50 g, 8.51 mmol) in acetonitrile (30 mL) was added 1-iodopyrrolidine-2,5-dione (2.11 g, 9.36 mmol). The resulting mixture was stirred for 12 hours while slowly warming to room temperature, and was then concentrated under reduced pressure. The resulting crude product was purified by flash chromatography on silica gel (0-60% methanol in ethyl acetate) to provide the title compound: LCMS m/z 302.99 [M+H]⁺; ¹H NMR (400 MHz, CD₃OD) δ=8.44-8.38 (s, 1H), 7.67 (s, 1H), 7.61-7.56 (m, 1H), 7.54-7.50 (m, 1H), 1.69-1.55 (m, 6H).

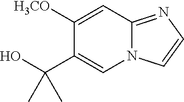

### Intermediate J, 2-(7-methoxyimidazo[1,2-a]pyridin-6-yl)propan-2-ol

### Step A. 5-bromo-4-methoxypyridin-2-amine

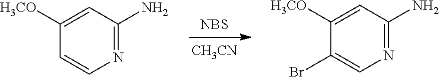

To a cooled 0° C. solution of 4-methoxypyridin-2-amine (106.0 g, 853.9 mmol) in acetonitrile (2000 mL) was added 1-bromopyrrolidine-2,5-dione (155.0 g, 871.0 mmol). The resulting mixture was stirred for 2 hours while slowly warming to room temperature. The reaction was then concentrated under reduced pressure, diluted with saturated aqueous sodium bicarbonate solution (600 mL), and extracted with dichloromethane (2×500 mL). The combined organic extracts were washed with saturated aqueous sodium chloride solution (2×300 mL), dried over sodium sulfate, filtered, and concentrated under reduced pressure to provide the title compound: ¹H NMR (400 MHz, DMSO-d₆) δ 7.84 (s, 1H), 6.13 (s, 1H), 6.05 (br s, 2H), 3.80 (s, 3H).

### Step B. 6-bromo-7-methoxyimidazo[1,2-a]pyridine

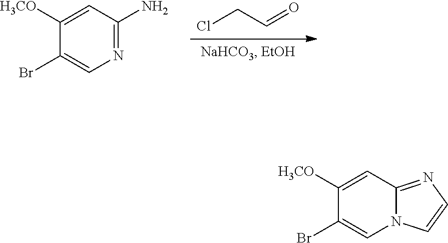

To a solution of 5-bromo-4-methoxypyridin-2-amine (10.0 g, 49.3 mmol) and 2-chloroacetaldehyde (48.33 g, 246.3 mmol, 39.61 mL) in ethanol (150 mL) was added sodium bicarbonate (10.34 g, 123.1 mmol). The resulting reaction mixture was heated at 80° C. for 15 hours. The reaction mixture was then cooled to room temperature, diluted with water (100 mL), and extracted with ethyl acetate (3×100 mL). The combined organic extracts were washed with saturated aqueous sodium chloride solution (2×100 mL), dried over sodium sulfate, filtered, and concentrated under reduced pressure. The resulting crude product was purified by flash chromatography on silica gel (0-10% methanol in dichloromethane) to provide the title compound: LCMS m/z 227.0 [M+H]⁺; ¹H NMR (400 MHz, DMSO-d₆) δ 9.03-8.74 (m, 1H), 7.78-7.62 (m, 1H), 7.44 (s, 1H), 7.09 (s, 1H), 3.91 (s, 3H).

### Step C. Methyl 7-methoxyimidazo[1,2-a]pyridine-6-carboxylate

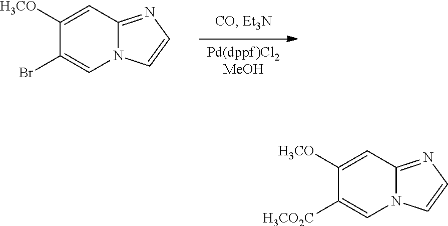 [SMILES: COc1cc2nccn2cc1Br ; "Pd(dppf)Cl2MeOH" ; COC(=O)c1cn2ccnc2cc1OC]

To a solution of 6-bromo-7-methoxyimidazo[1,2-a]pyridine (8.00 g, 35.2 mmol) in methanol (250 mL) and toluene (250 mL) were added triethylamine (10.70 g, 105.7 mmol) and [1,1-bis(diphenylphosphino)ferrocene]dichloropalladium(II) (2.58 g, 3.52 mmol), in that order. The resulting reaction mixture was heated at 80° C. under a carbon monoxide atmosphere (3 MPa) for 16 hours. The reaction mixture was then cooled to room temperature and concentrated under reduced pressure. The resulting crude product was purified by flash chromatography on silica gel (0-20% methanol in ethyl acetate) to provide the title compound: LCMS m/z 207.2 [M+H]⁺; ¹H NMR (400 MHz, CDCl₃) δ 8.69 (s, 1H), 7.54 (s, 1H), 7.46 (s, 1H), 6.92 (s, 1H), 3.92 (s, 3H), 3.90 (s, 3H).

### Step D. 2-(7-methoxyimidazo[1,2-a]pyridin-6-yl)propan-2-ol

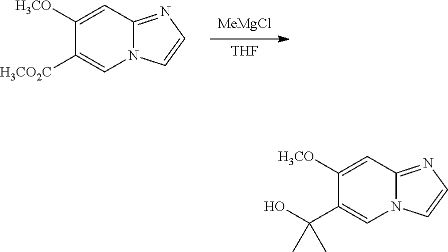

To a cooled 0° C. solution of methyl 7-methoxyimidazo[1,2-a]pyridine-6-carboxylate (3.00 g, 14.6 mmol) in tetrahydrofuran (100 mL) was added a solution of methyl magnesium bromide in diethyl ether (3.0 M, 19.4 mL, 58.2 mmol). The resulting reaction mixture was stirred for 2 hours while slowly warming to room temperature. The reaction mixture was then quenched by addition water (20 mL) at 0° C., and extracted with ethyl acetate (3×30 mL). The combined organic extracts were washed with saturated aqueous sodium chloride solution (2×15 mL), dried over anhydrous sodium sulfate, filtered, and concentrated under reduced pressure to provide the title compound: ¹H NMR (400 MHz, CD₃OD) δ 8.40 (s, 1H), 7.62 (s, 1H), 7.33 (s, 1H), 6.86 (s, 1H), 3.89-3.85 (s, 3H), 1.61-1.53 (m, 6H).

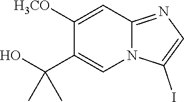 [SMILES: COc1cc2ncc(I)n2cc1C(C)(C)O]

### Intermediate K, 2-(3-iodo-7-methoxyimidazo[1,2-a]pyridin-6-yl)propan-2-ol

### Step A. 2-(3-iodo-7-methoxyimidazo[1,2-a]pyridin-6-yl)propan-2-ol

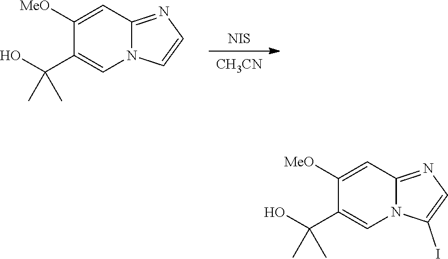

To a cooled 0° C. solution of 2-(7-methoxyimidazo[1,2-a]pyridin-6-yl)propan-2-ol (1.00 g, 4.85 mmol) in acetonitrile (10 mL) was added 1-iodopyrrolidine-2,5-dione (1.09 g, 4.85 mmol). The resulting mixture was stirred for 2 hours while warming to room temperature, and was then concentrated under reduced pressure. The crude product thus obtained was triturated with ethyl acetate to give a mixture that was then filtered. The solids were collected and dried under reduced pressure to provide the title compound: LCMS m/z 332.9 [M+H]⁺; ¹H NMR (400 MHz, CD₃OD) δ 8.45-8.37 (m, 1H), 7.56-7.44 (m, 1H), 7.02-6.93 (m, 1H), 3.99 (s, 3H), 1.69-1.61 (m, 6H).

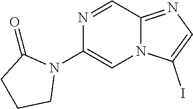 [SMILES: O=C1CCCN1c1cn2c(I)cnc2cn1]

### Intermediate L, 1-(3-iodoimidazo[1,2-a]pyrazin-6-yl)pyrrolidin-2-one

### Step A. 1-(imidazo[1,2-a]pyrazin-6-yl)pyrrolidin-2-one

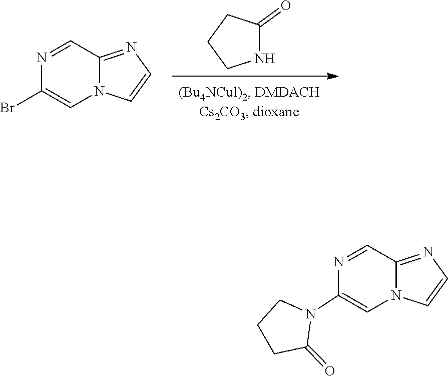 [SMILES: Brc1cn2ccnc2cn1 ; O=C1CCCN1 ; O=C1CCCN1c1cn2ccnc2cn1]

A mixture of 6-bromoimidazo[1,2-a]pyrazine (5.00 g, 25.3 mmol), pyrrolidin-2-one (2.58 g, 30.3 mmol), cesium carbonate (24.7 g, 75.8 mmol), (1R,2R)—N1,N2-dimethylcyclohexane-1,2-diamine (3.59 g, 25.4 mmol), and bis[(tetrabutylammonium iodide)copper(I) iodide](1.41 g, 1.26 mmol) in dioxane (70 mL) was purged with nitrogen, and was then heated at 120° C. for 2 hours under nitrogen atmosphere. The reaction was then cooled to room temperature, diluted with water (50 mL), and extracted with ethyl acetate (3×50 mL). The organic extracts were combined, washed with saturated aqueous sodium chloride solution (20 mL), dried over sodium sulfate, filtered, and concentrated under reduced pressure. The resulting crude product was purified by flash silica gel chromatography on silica gel (0-80% ethyl acetate in petroleum ether) to provide the title compound: LCMS m/z 203.3 [M+H]⁺; ¹H NMR (400 MHz, CD₃OD), δ 9.31-9.41 (m, 1H), 8.82-8.91 (m, 1H), 8.03-8.11 (m, 1H), 7.80 (d, 1H), 4.11-4.20 (m, 2H), 2.67 (t, 2H), 2.17-2.24 (m, 2H).

### Step B. 1-(3-iodoimidazo[1,2-a]pyrazin-6-yl)pyrrolidin-2-one

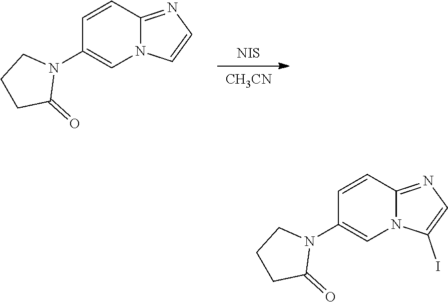

To a solution of 1-(imidazo[1,2-a]pyrazin-6-yl)pyrrolidin-2-one (0.680 g, 3.36 mmol) in acetonitrile (10 mL) was added 1-iodopyrrolidine-2,5-dione (0.757 g, 3.36 mmol). The resulting mixture was stirred at room temperature for 16 hours, and was then filtered, rinsed with ethyl acetate (3×10 mL), and dried under reduced pressure to provide the title compound: LCMS m/z 329.1 [M+H]⁺; ¹H NMR (400 MHz, CD₃OD) δ 9.36-9.35 (s, 1H), 8.80 (s, 1H), 7.89 (s, 1H), 4.21-4.17 (t, J=7.2 Hz, 2H), 2.72-2.70 (m, 2H), 2.24-2.20 (m, 2H).

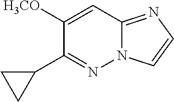 [SMILES: COc1cc2nccn2nc1C1CC1]

### Intermediate M, 6-cyclopropyl-7-methoxyimidazo[1,2-b]pyridazine

### Step A. 6-chloro-3-cyclopropyl-4-methoxypyridazine

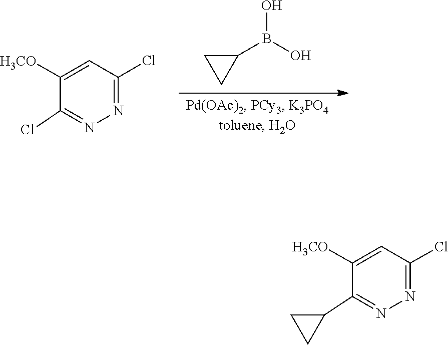 [SMILES: COc1cc(Cl)nnc1Cl ; OB(O)C1CC1 ; COc1cc(Cl)nnc1C1CC1]

A mixture of 3,6-dichloro-4-methoxy-pyridazine (10.00 g, 55.86 mmol), cyclopropylboronic acid (7.20 g, 83.8 mmol), potassium phosphate (35.57 g, 167.6 mmol), palladium(II)acetate (1.25 g, 5.59 mmol), and tricyclohexylphosphine (2.35 g, 8.38 mmol, 2.72 mL) in toluene (100 mL) and water (10 mL) was degassed and purged with nitrogen three times, and was then stirred at 100° C. for 16 hours under nitrogen atmosphere. The reaction was then cooled to room temperature and concentrated under reduced pressure. The resulting crude product was diluted with water (50 mL) and extracted with ethyl acetate (3×80 mL). The combined organic layers were then washed with saturated aqueous sodium chloride solution (60 mL), dried over sodium sulfate, filtered, and concentrated under reduced pressure. The resulting crude product was purified by flash chromatography on silica gel (0-20% ethyl acetate in petroleum ether) to provide the title compound: ¹H NMR (400 MHz, CDCl₃) δ 6.69 (s, 1H), 3.87 (s, 3H), 2.33-2.25 (m, 1H), 1.22-1.17 (m, 2H), 1.07-0.96 (m, 2H).

### Step B. 6-cyclopropyl-5-methoxy-N-(4-methoxybenzyl)pyridazin-3-amine

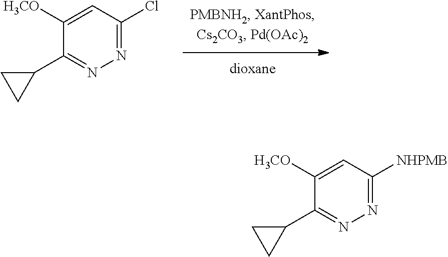

A mixture of 6-chloro-3-cyclopropyl-4-methoxy-pyridazine (4.00 g, 21.7 mmol), (4-methoxyphenyl)methanamine (8.92 g, 65.0 mmol, 8.41 mL), 4,5-bis(diphenylphosphino)-9,9-dimethyl-9H-xanthene (1.88 g, 3.25 mmol), palladium(II)acetate (0.730 g, 3.25 mmol), and cesium carbonate (21.18 g, 65.00 mmol) in dioxane (50 mL) was degassed and purged with nitrogen three times, and was then stirred at 120° C. for 16 hours under nitrogen atmosphere. The reaction was then cooled to room temperature, diluted with water (40 mL), and extracted with ethyl acetate (3×40 mL). The combined organic layers were washed with saturated aqueous sodium chloride solution (50 mL), dried over sodium sulfate, filtered, and concentrated under reduced pressure. The resulting crude product was purified by flash chromatography on silica gel (0-30% ethyl acetate in petroleum ether) to provide the title compound: LCMS m/z 286.2 [M+H]⁺.

### Step C. 6-cyclopropyl-5-methoxypyridazin-3-amine

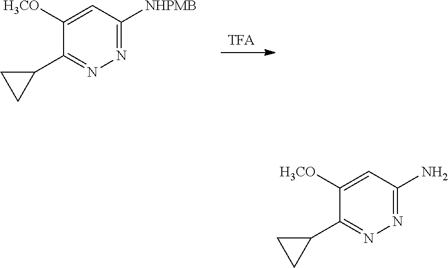

A solution of 6-cyclopropyl-5-methoxy-N-[(4-methoxyphenyl)methyl]pyridazin-3-amine (3.00 g, 10.5 mmol) in trifluoroacetic acid (30 mL) was stirred at room temperature for 16 hours. Methanol was added, causing precipitation of a solid. The resulting mixture was filtered, and the mother liquor was concentrated under reduced pressure to provide the title compound: LCMS m/z 166.1 [M+H]⁺.

### Step D. 6-cyclopropyl-7-methoxyimidazo[1,2-b]pyridazine

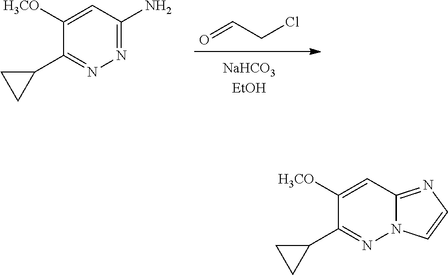

To a solution of 6-cyclopropyl-5-methoxy-pyridazin-3-amine (1.00 g, 6.05 mmol) and 2-chloroacetaldehyde (4.75 g, 60.5 mmol, 3.89 mL) in ethanol (2 mL) was added sodium bicarbonate (1.02 g, 12.1 mmol). The resulting reaction mixture was stirred at 80° C. for 4 hours, and was then cooled to room temperature and concentrated under reduced pressure. The resulting residue was diluted with water (10 mL) and extracted with ethyl acetate (3×10 mL). The combined organic layers were then washed with saturated aqueous sodium chloride solution (5 mL), dried over sodium sulfate, filtered, and concentrated under reduced pressure. The resulting crude product was purified by flash chromatography on silica gel (0-50% ethyl acetate in petroleum ether) to give the title compound: LCMS m/z 166.1 [M+H]⁺; ¹H NMR (400 MHz, CDCl₃) δ 7.64-7.52 (m, 1H), 7.40-7.29 (m, 1H), 7.02 (s, 1H), 3.88 (s, 3H), 2.44-2.19 (m, 1H), 1.10-1.02 (m, 2H), 1.00-0.92 (m, 2H).

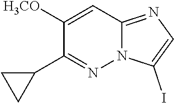 [SMILES: COc1cc2ncc(I)n2nc1C1CC1]

### Intermediate N, 6-cyclopropyl-3-iodo-7-methoxyimidazo[1,2-b]pyridazine

### Step A. 6-cyclopropyl-3-iodo-7-methoxyimidazo[1,2-b]pyridazine

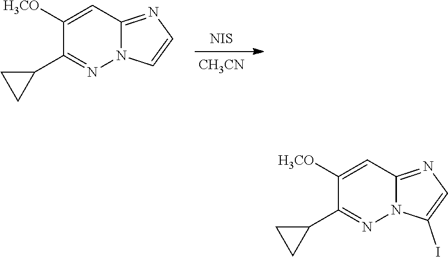

To a cooled 0° C. solution of 6-cyclopropyl-7-methoxy-imidazo[1,2-b]pyridazine (0.450 g, 2.38 mmol) in acetonitrile (5 mL) was added 1-iodopyrrolidine-2,5-dione (1.07 g, 4.76 mmol). The resulting mixture was stirred for 3 hours while warming to room temperature. The mixture was then filtered and concentrated under reduced pressure. The crude product thus obtained was triturated with ethyl acetate (2×5 mL) to give a mixture that was filtered and dried under reduced pressure to provide the title compound: LCMS m/z 346.1 [M+H]⁺; ¹H NMR (400 MHz, DMSO-d₆) δ 7.58 (s, 1H), 7.44 (s, 1H), 3.96 (s, 1H), 2.90-2.71 (in, 1H), 1.08-1.06 (m, 4H).

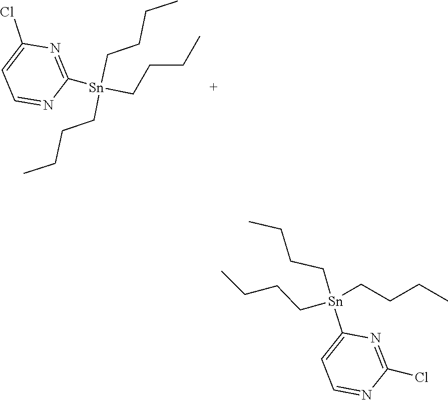 [SMILES: CCC[CH2][Sn]([CH2]CCC)([CH2]CCC)[c]1nccc(Cl)n1 ; CCC[CH2][Sn]([CH2]CCC)([CH2]CCC)[c]1ccnc(Cl)n1]

### Intermediate O, 4-chloro-2-(tributylstannyl)pyrimidine; and Intermediate P, 2-chloro-4-(tributylstannyl)pyrimidine

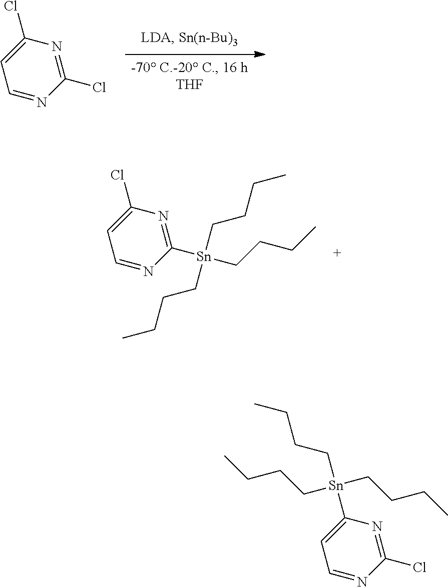 [SMILES: Clc1ccnc(Cl)n1 ; CCC[CH2][Sn]([CH2]CCC)([CH2]CCC)[c]1nccc(Cl)n1 ; CCC[CH2][Sn]([CH2]CCC)([CH2]CCC)[c]1ccnc(Cl)n1]

To a cooled 0° C. solution of lithium diisopropylamide (2.0 M in tetrahydrofuran, 25.17 mL) in tetrahydrofuran (50 mL) was added tributylstannane (11.68 g, 40.27 mmol, 10.62 mL). The resulting reaction was stirred at 0° C. for 30 minutes, and was then cooled to −70° C. A solution of 2,4-dichloropyrimidine (5.00 g, 33.6 mmol) in tetrahydrofuran (20 mL) was then added in a dropwise manner. The reaction was stirred at −70° C. for 4 hours, and was then warmed to room temperature and stirred for an additional 12 hours. The reaction was then quenched by addition of saturated aqueous ammonium chloride solution (50 mL). The resulting mixture was stirred at room temperature for 30 minutes, and was then extracted with ethyl acetate (3×60 mL). The organic extracts were combined, washed with saturated aqueous sodium chloride solution (50 mL), dried over sodium sulfate, filtered, and concentrated under reduced pressure. The resulting crude product was purified by flash chromatography on silica gel (0-10% ethyl acetate in petroleum ether) to give a product that was further purified by HPLC (Welch Xtimate C18 column, 10 micron, 250×70 mm; 5-10% acetonitrile in aqueous 10 mM ammonium bicarbonate) to provide the title compounds:

4-chloro-2-(tributylstannyl)pyrimidine: LCMS m/z 405.1 [M+H]⁺; ¹H NMR (400 MHz, CDCl₃) δ 8.46 (d, J=5.5 Hz, 1H), 7.14-7.00 (m, 1H), 1.62-1.43 (m, 6H), 1.30-1.22 (m, 6H), 1.20-1.04 (m, 6H), 0.81 (t, J=7.3 Hz, 9H).

2-chloro-4-(tributylstannyl)pyrimidine: LCMS m/z 405.1 [M+H]⁺; ¹H NMR (400 MHz, CDCl₃) δ 8.35 (d, J=4.8 Hz, 1H), 7.34 (d, J=5.2 Hz, 1H), 1.63-1.44 (m, 6H), 1.38 (br s, 6H), 1.22-1.07 (m, 6H), 0.89 (t, J=7.2 Hz, 9H).

### Example 2

### Exemplary Compounds 1-84 of the Disclosure

Example 2 provides exemplary compounds of the disclosure in Table 1 prepared using the synthetic procedures described herein.

TABLE 1
Exemplary compounds of the disclosure

| Compound | |
|---|---|
| # | Structure |
| 1 | 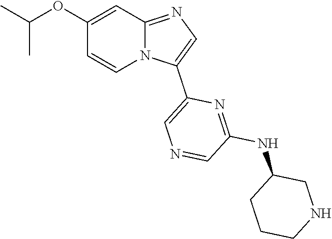 [SMILES: CC(C)Oc1ccn2c(-c3cncc(N[C@@H]4CCCNC4)n3)cnc2c1] |
| 2 | 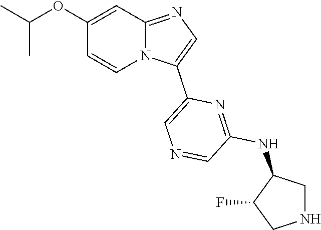 [SMILES: CC(C)Oc1ccn2c(-c3cncc(N[C@H]4CNC[C@@H]4F)n3)cnc2c1] |
| 3 | 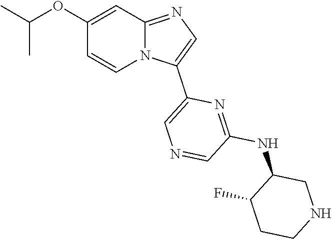 [SMILES: CC(C)Oc1ccn2c(-c3cncc(N[C@H]4CNCC[C@@H]4F)n3)cnc2c1] |
| 4 | 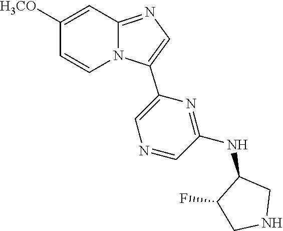 [SMILES: COc1ccn2c(-c3cncc(N[C@H]4CNC[C@@H]4F)n3)cnc2c1] |
| 5 | 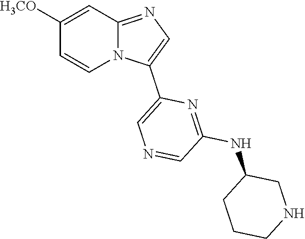 [SMILES: COc1ccn2c(-c3cncc(N[C@@H]4CCCNC4)n3)cnc2c1] |
| 6 | 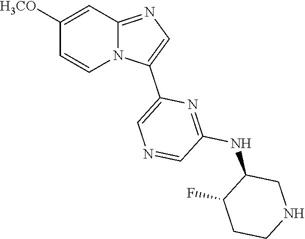 [SMILES: COc1ccn2c(-c3cncc(N[C@H]4CNCC[C@@H]4F)n3)cnc2c1] |
| 7 | 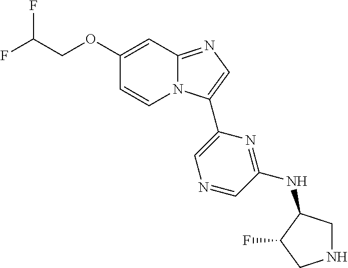 [SMILES: FC(F)COc1ccn2c(-c3cncc(N[C@H]4CNC[C@@H]4F)n3)cnc2c1] |
| 8 | 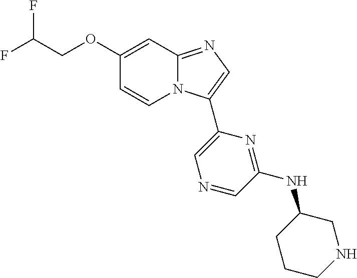 [SMILES: FC(F)COc1ccn2c(-c3cncc(N[C@@H]4CCCNC4)n3)cnc2c1] |
| 9 | 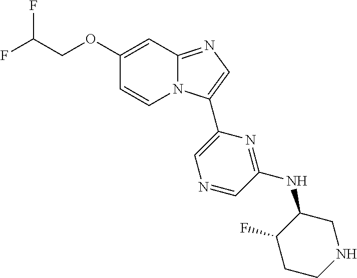 [SMILES: FC(F)COc1ccn2c(-c3cncc(N[C@H]4CNCC[C@@H]4F)n3)cnc2c1] |
| 10 | 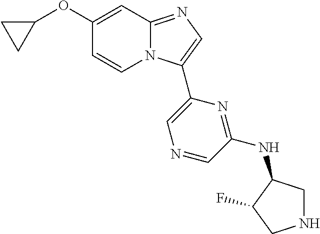 [SMILES: F[C@H]1CNC[C@@H]1Nc1cncc(-c2cnc3cc(OC4CC4)ccn23)n1] |
| 11 | 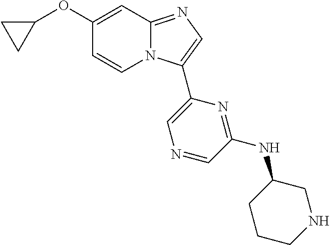 [SMILES: c1cn2c(-c3cncc(N[C@@H]4CCCNC4)n3)cnc2cc1OC1CC1] |
| 12 | 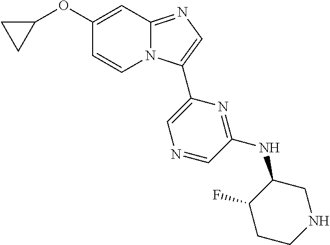 [SMILES: F[C@H]1CCNC[C@@H]1Nc1cncc(-c2cnc3cc(OC4CC4)ccn23)n1] |
| 13 | 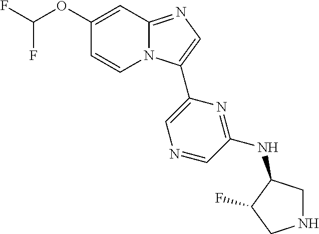 [SMILES: FC(F)Oc1ccn2c(-c3cncc(N[C@H]4CNC[C@@H]4F)n3)cnc2c1] |
| 14 | 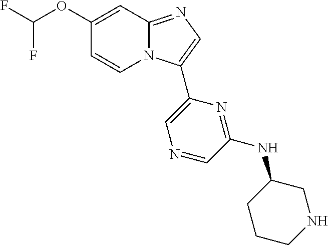 [SMILES: FC(F)Oc1ccn2c(-c3cncc(N[C@@H]4CCCNC4)n3)cnc2c1] |
| 15 | 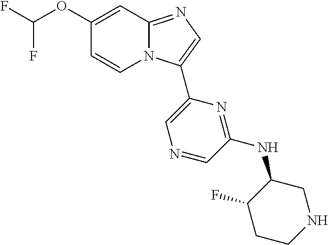 [SMILES: FC(F)Oc1ccn2c(-c3cncc(N[C@H]4CNCC[C@@H]4F)n3)cnc2c1] |
| 16 | 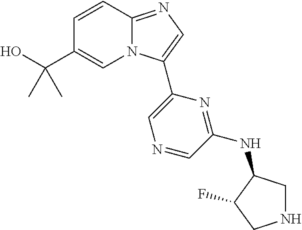 [SMILES: CC(C)(O)c1ccc2ncc(-c3cncc(N[C@H]4CNC[C@@H]4F)n3)n2c1] |
| 17 | 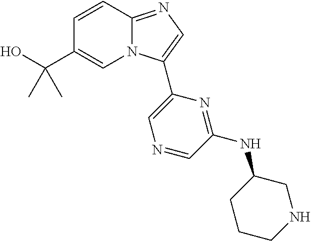 [SMILES: CC(C)(O)c1ccc2ncc(-c3cncc(N[C@@H]4CCCNC4)n3)n2c1] |
| 18 | 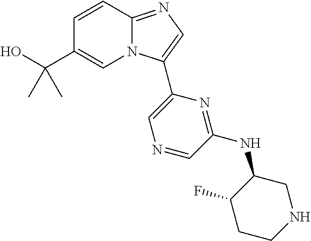 [SMILES: CC(C)(O)c1ccc2ncc(-c3cncc(N[C@H]4CNCC[C@@H]4F)n3)n2c1] |
| 19 | 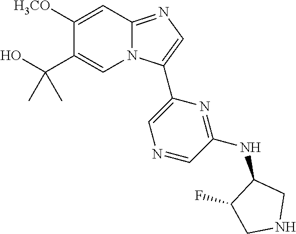 [SMILES: COc1cc2ncc(-c3cncc(N[C@H]4CNC[C@@H]4F)n3)n2cc1C(C)(C)O] |
| 20 | 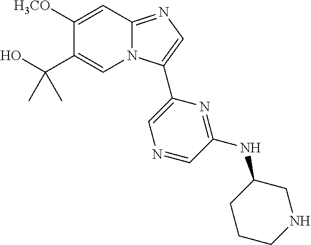 [SMILES: COc1cc2ncc(-c3cncc(N[C@@H]4CCCNC4)n3)n2cc1C(C)(C)O] |
| 21 | 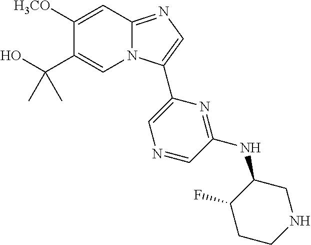 [SMILES: COc1cc2ncc(-c3cncc(N[C@H]4CNCC[C@@H]4F)n3)n2cc1C(C)(C)O] |
| 22 | 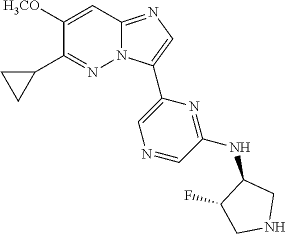 [SMILES: COc1cc2ncc(-c3cncc(N[C@H]4CNC[C@@H]4F)n3)n2nc1C1CC1] |
| 23 | 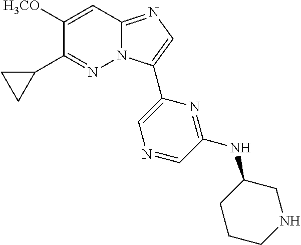 [SMILES: COc1cc2ncc(-c3cncc(N[C@@H]4CCCNC4)n3)n2nc1C1CC1] |
| 24 | 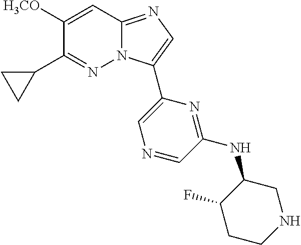 [SMILES: COc1cc2ncc(-c3cncc(N[C@H]4CNCC[C@@H]4F)n3)n2nc1C1CC1] |
| 25 | 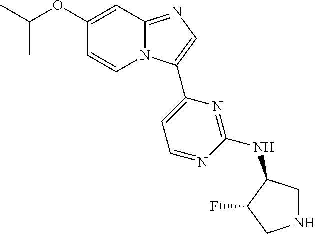 [SMILES: CC(C)Oc1ccn2c(-c3ccnc(N[C@H]4CNC[C@@H]4F)n3)cnc2c1] |
| 26 | 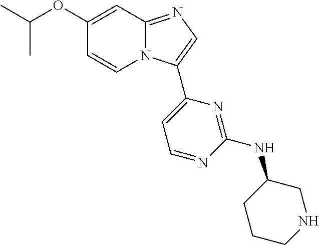 [SMILES: CC(C)Oc1ccn2c(-c3ccnc(N[C@@H]4CCCNC4)n3)cnc2c1] |
| 27 | 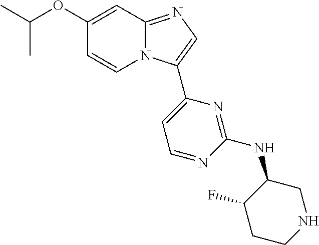 [SMILES: CC(C)Oc1ccn2c(-c3ccnc(N[C@H]4CNCC[C@@H]4F)n3)cnc2c1] |
| 28 | 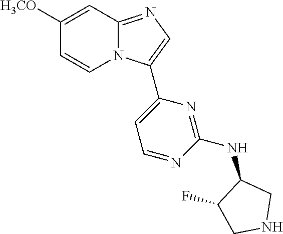 [SMILES: COc1ccn2c(-c3ccnc(N[C@H]4CNC[C@@H]4F)n3)cnc2c1] |
| 29 | 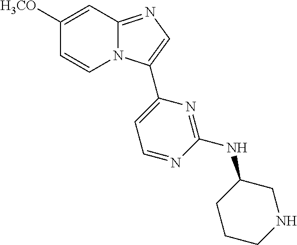 [SMILES: COc1ccn2c(-c3ccnc(N[C@@H]4CCCNC4)n3)cnc2c1] |
| 30 | 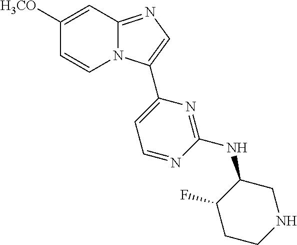 [SMILES: COc1ccn2c(-c3ccnc(N[C@H]4CNCC[C@@H]4F)n3)cnc2c1] |
| 31 | 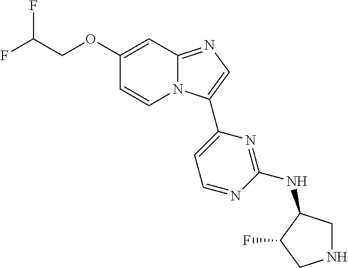 [SMILES: FC(F)COc1ccn2c(-c3ccnc(N[C@H]4CNC[C@@H]4F)n3)cnc2c1] |
| 32 | 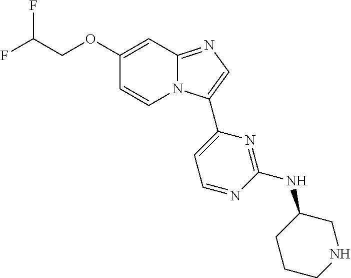 [SMILES: FC(F)COc1ccn2c(-c3ccnc(N[C@@H]4CCCNC4)n3)cnc2c1] |
| 33 | 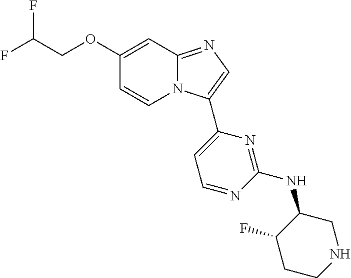 [SMILES: FC(F)COc1ccn2c(-c3ccnc(N[C@H]4CNCC[C@@H]4F)n3)cnc2c1] |
| 34 | 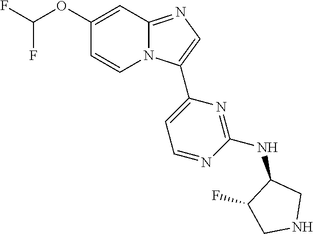 [SMILES: FC(F)Oc1ccn2c(-c3ccnc(N[C@H]4CNC[C@@H]4F)n3)cnc2c1] |
| 35 | 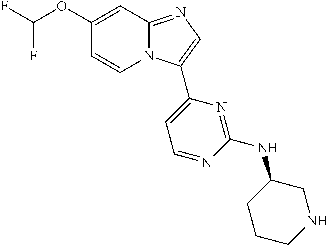 [SMILES: FC(F)Oc1ccn2c(-c3ccnc(N[C@@H]4CCCNC4)n3)cnc2c1] |
| 36 | 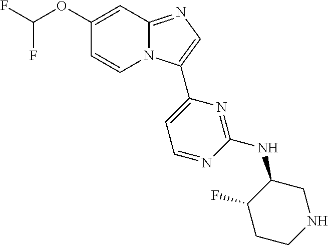 [SMILES: FC(F)Oc1ccn2c(-c3ccnc(N[C@H]4CNCC[C@@H]4F)n3)cnc2c1] |
| 37 | 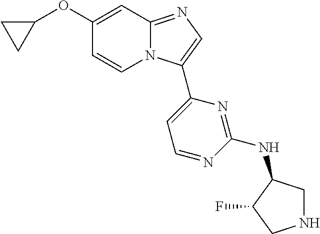 [SMILES: F[C@H]1CNC[C@@H]1Nc1nccc(-c2cnc3cc(OC4CC4)ccn23)n1] |
| 38 | 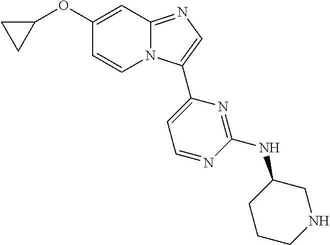 [SMILES: c1cc(-c2cnc3cc(OC4CC4)ccn23)nc(N[C@@H]2CCCNC2)n1] |
| 39 | 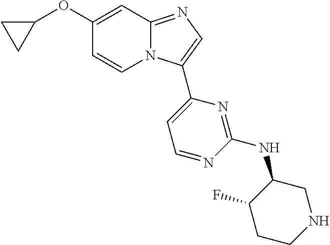 [SMILES: F[C@H]1CCNC[C@@H]1Nc1nccc(-c2cnc3cc(OC4CC4)ccn23)n1] |
| 40 | 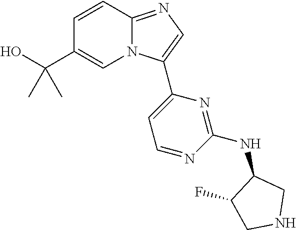 [SMILES: CC(C)(O)c1ccc2ncc(-c3ccnc(N[C@H]4CNC[C@@H]4F)n3)n2c1] |
| 41 | 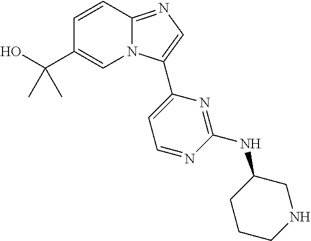 [SMILES: CC(C)(O)c1ccc2ncc(-c3ccnc(N[C@@H]4CCCNC4)n3)n2c1] |
| 43 | 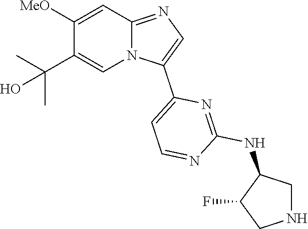 [SMILES: COc1cc2ncc(-c3ccnc(N[C@H]4CNC[C@@H]4F)n3)n2cc1C(C)(C)O] |
| 44 | 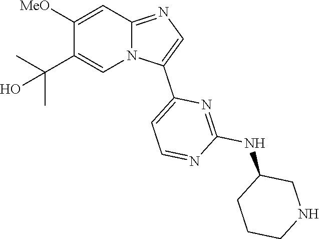 [SMILES: COc1cc2ncc(-c3ccnc(N[C@@H]4CCCNC4)n3)n2cc1C(C)(C)O] |
| 45 | 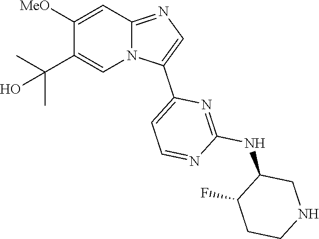 [SMILES: COc1cc2ncc(-c3ccnc(N[C@H]4CNCC[C@@H]4F)n3)n2cc1C(C)(C)O] |
| 46 | 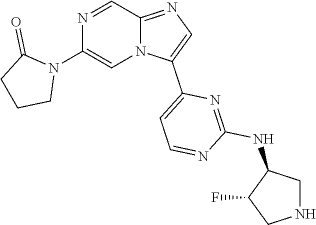 [SMILES: O=C1CCCN1c1cn2c(-c3ccnc(N[C@H]4CNC[C@@H]4F)n3)cnc2cn1] |
| 47 | 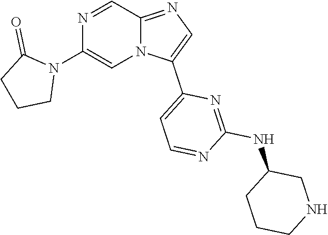 [SMILES: O=C1CCCN1c1cn2c(-c3ccnc(N[C@@H]4CCCNC4)n3)cnc2cn1] |
| 48 | 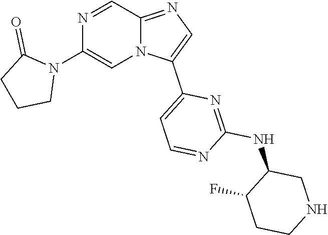 [SMILES: O=C1CCCN1c1cn2c(-c3ccnc(N[C@H]4CNCC[C@@H]4F)n3)cnc2cn1] |
| 49 | 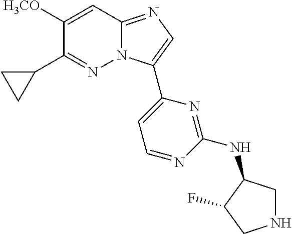 [SMILES: COc1cc2ncc(-c3ccnc(N[C@H]4CNC[C@@H]4F)n3)n2nc1C1CC1] |
| 50 | 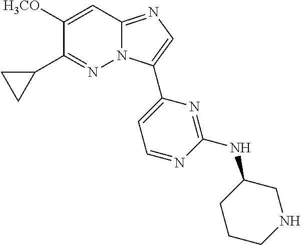 [SMILES: COc1cc2ncc(-c3ccnc(N[C@@H]4CCCNC4)n3)n2nc1C1CC1] |
| 51 | 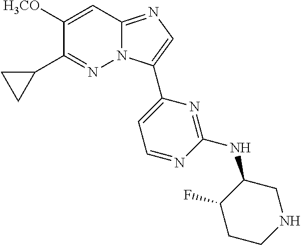 [SMILES: COc1cc2ncc(-c3ccnc(N[C@H]4CNCC[C@@H]4F)n3)n2nc1C1CC1] |
| 52 | 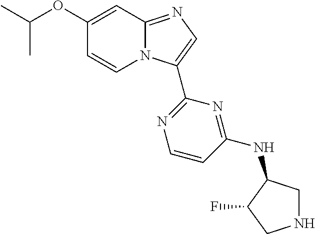 [SMILES: CC(C)Oc1ccn2c(-c3nccc(N[C@H]4CNC[C@@H]4F)n3)cnc2c1] |
| 53 | 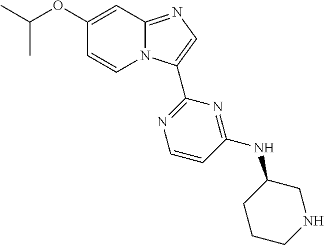 [SMILES: CC(C)Oc1ccn2c(-c3nccc(N[C@@H]4CCCNC4)n3)cnc2c1] |
| 54 | 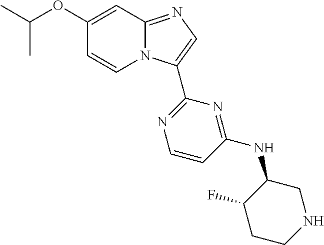 [SMILES: CC(C)Oc1ccn2c(-c3nccc(N[C@H]4CNCC[C@@H]4F)n3)cnc2c1] |
| 55 | 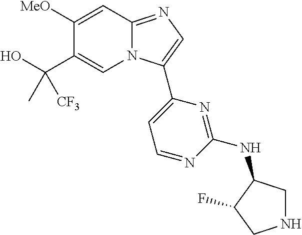 [SMILES: COc1cc2ncc(-c3ccnc(N[C@H]4CNC[C@@H]4F)n3)n2cc1C(C)(O)C(F)(F)F] |
| 56 | 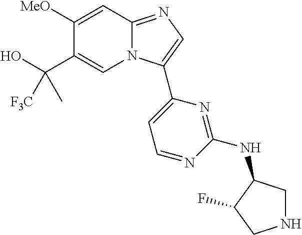 [SMILES: COc1cc2ncc(-c3ccnc(N[C@H]4CNC[C@@H]4F)n3)n2cc1C(C)(O)C(F)(F)F] |
| 57 | 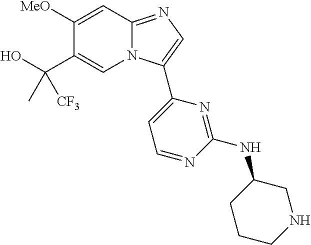 [SMILES: COc1cc2ncc(-c3ccnc(N[C@@H]4CCCNC4)n3)n2cc1C(C)(O)C(F)(F)F] |
| 58 | 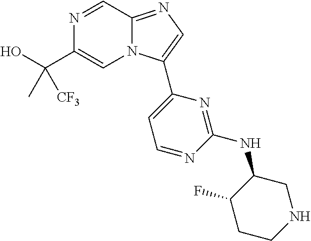 [SMILES: CC(O)(c1cn2c(-c3ccnc(N[C@H]4CNCC[C@@H]4F)n3)cnc2cn1)C(F)(F)F] |
| 59 | 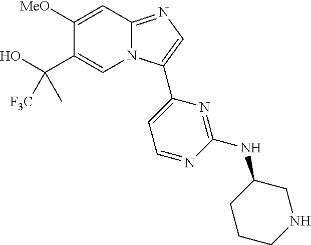 [SMILES: COc1cc2ncc(-c3ccnc(N[C@@H]4CCCNC4)n3)n2cc1C(C)(O)C(F)(F)F] |
| 60 | 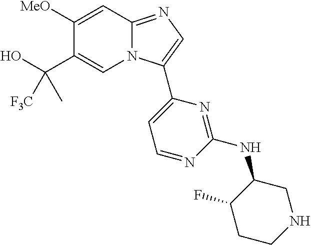 [SMILES: COc1cc2ncc(-c3ccnc(N[C@H]4CNCC[C@@H]4F)n3)n2cc1C(C)(O)C(F)(F)F] |
| 61 | 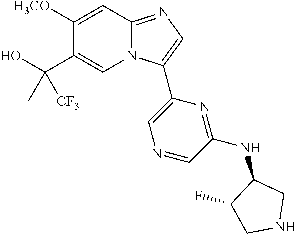 [SMILES: COc1cc2ncc(-c3cncc(N[C@H]4CNC[C@@H]4F)n3)n2cc1C(C)(O)C(F)(F)F] |
| 62 | 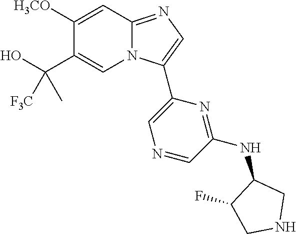 [SMILES: COc1cc2ncc(-c3cncc(N[C@H]4CNC[C@@H]4F)n3)n2cc1C(C)(O)C(F)(F)F] |
| 63 | 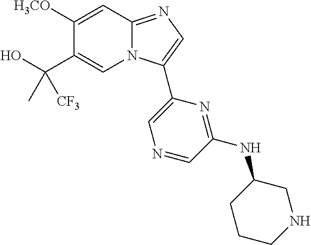 [SMILES: COc1cc2ncc(-c3cncc(N[C@@H]4CCCNC4)n3)n2cc1C(C)(O)C(F)(F)F] |
| 64 | 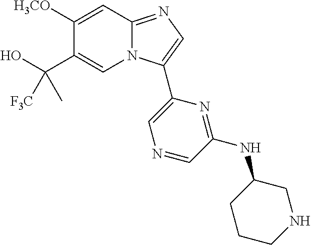 [SMILES: COc1cc2ncc(-c3cncc(N[C@@H]4CCCNC4)n3)n2cc1C(C)(O)C(F)(F)F] |
| 65 | 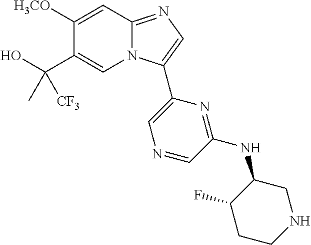 [SMILES: COc1cc2ncc(-c3cncc(N[C@H]4CNCC[C@@H]4F)n3)n2cc1C(C)(O)C(F)(F)F] |
| 66 | 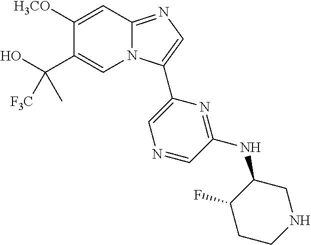 [SMILES: COc1cc2ncc(-c3cncc(N[C@H]4CNCC[C@@H]4F)n3)n2cc1C(C)(O)C(F)(F)F] |
| 67 | 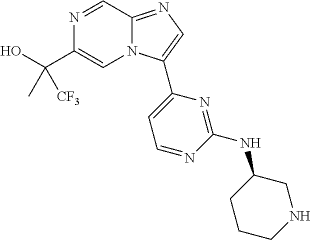 [SMILES: CC(O)(c1cn2c(-c3ccnc(N[C@@H]4CCCNC4)n3)cnc2cn1)C(F)(F)F] |
| 68 | 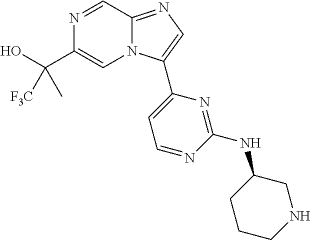 [SMILES: CC(O)(c1cn2c(-c3ccnc(N[C@@H]4CCCNC4)n3)cnc2cn1)C(F)(F)F] |
| 69 | 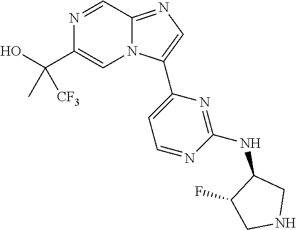 [SMILES: CC(O)(c1cn2c(-c3ccnc(N[C@H]4CNC[C@@H]4F)n3)cnc2cn1)C(F)(F)F] |
| 70 | 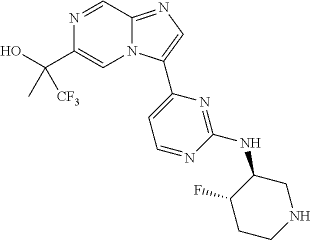 [SMILES: CC(O)(c1cn2c(-c3ccnc(N[C@H]4CNCC[C@@H]4F)n3)cnc2cn1)C(F)(F)F] |
| 71 | 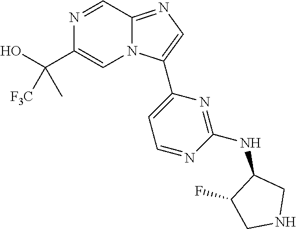 [SMILES: CC(O)(c1cn2c(-c3ccnc(N[C@H]4CNC[C@@H]4F)n3)cnc2cn1)C(F)(F)F] |
| 72 | 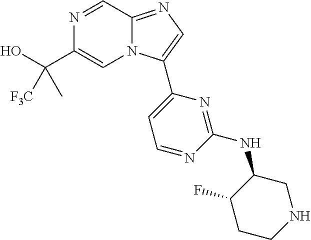 [SMILES: CC(O)(c1cn2c(-c3ccnc(N[C@H]4CNCC[C@@H]4F)n3)cnc2cn1)C(F)(F)F] |
| 73 | 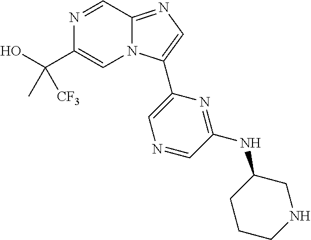 [SMILES: CC(O)(c1cn2c(-c3cncc(N[C@@H]4CCCNC4)n3)cnc2cn1)C(F)(F)F] |
| 74 | 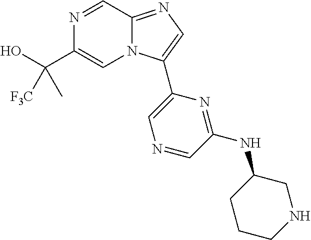 [SMILES: CC(O)(c1cn2c(-c3cncc(N[C@@H]4CCCNC4)n3)cnc2cn1)C(F)(F)F] |
| 75 | 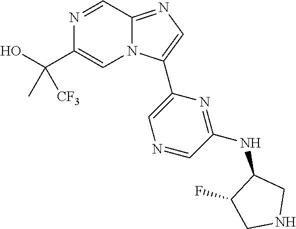 [SMILES: CC(O)(c1cn2c(-c3cncc(N[C@H]4CNC[C@@H]4F)n3)cnc2cn1)C(F)(F)F] |
| 76 | 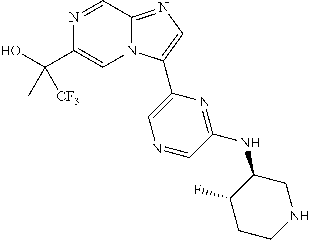 [SMILES: CC(O)(c1cn2c(-c3cncc(N[C@H]4CNCC[C@@H]4F)n3)cnc2cn1)C(F)(F)F] |
| 77 | 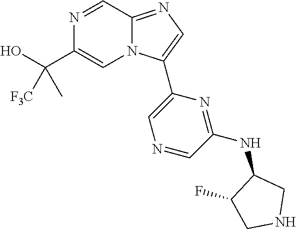 [SMILES: CC(O)(c1cn2c(-c3cncc(N[C@H]4CNC[C@@H]4F)n3)cnc2cn1)C(F)(F)F] |
| 78 | 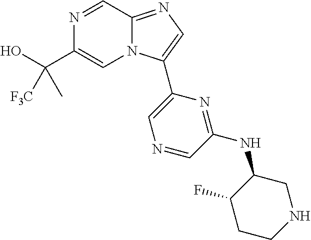 [SMILES: CC(O)(c1cn2c(-c3cncc(N[C@H]4CNCC[C@@H]4F)n3)cnc2cn1)C(F)(F)F] |
| 79 | 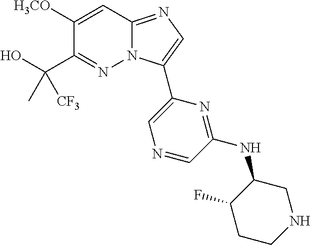 [SMILES: COc1cc2ncc(-c3cncc(N[C@H]4CNCC[C@@H]4F)n3)n2nc1C(C)(O)C(F)(F)F] |
| 80 | 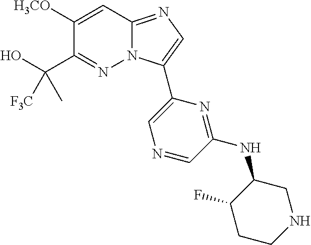 [SMILES: COc1cc2ncc(-c3cncc(N[C@H]4CNCC[C@@H]4F)n3)n2nc1C(C)(O)C(F)(F)F] |
| 81 | 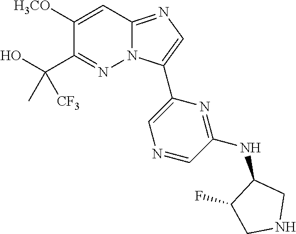 [SMILES: COc1cc2ncc(-c3cncc(N[C@H]4CNC[C@@H]4F)n3)n2nc1C(C)(O)C(F)(F)F] |
| 82 | 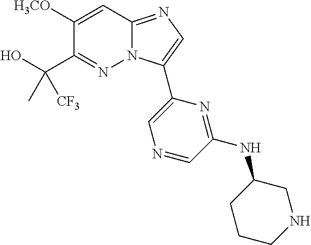 [SMILES: COc1cc2ncc(-c3cncc(N[C@@H]4CCCNC4)n3)n2nc1C(C)(O)C(F)(F)F] |
| 83 | 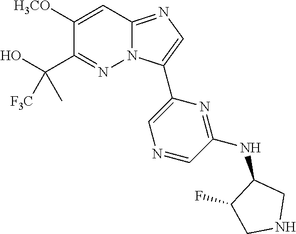 [SMILES: COc1cc2ncc(-c3cncc(N[C@H]4CNC[C@@H]4F)n3)n2nc1C(C)(O)C(F)(F)F] |
| 84 | 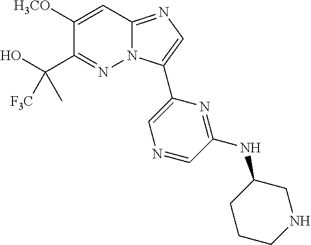 [SMILES: COc1cc2ncc(-c3cncc(N[C@@H]4CCCNC4)n3)n2nc1C(C)(O)C(F)(F)F] |

### Example 3

### Exemplary Synthetic Procedure #1 (Compounds 1a-3a)

### Compound 1a, 4-(6-cyclopropyl-7-methoxyimidazo[1,2-b]pyridazin-3-yl)-5-fluoro-N-((3S,4S)-4-fluoropiperidin-3-yl)pyrimidin-2-amine

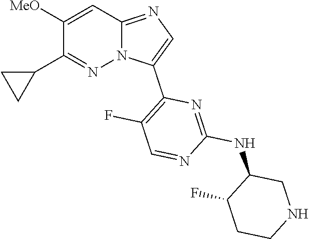 [SMILES: COc1cc2ncc(-c3nc(N[C@H]4CNCC[C@@H]4F)ncc3F)n2nc1C1CC1]

### Step A. 6-chloro-3-cyclopropyl-4-methoxypyridazine

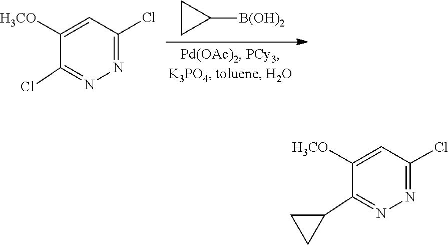

A mixture of 3,6-dichloro-4-methoxy-pyridazine (10.00 g, 55.86 mmol), cyclopropylboronic acid (7.20 g, 83.8 mmol), potassium phosphate (35.57 g, 167.6 mmol), palladium(II)acetate (1.25 g, 5.59 mmol), and tricyclohexylphosphine (2.35 g, 8.38 mmol, 2.72 mL) in toluene (100 mL) and water (10 mL) was degassed and purged with nitrogen three times, and was then stirred at 100° C. for 16 hours under nitrogen atmosphere. The reaction was then cooled to room temperature and concentrated under reduced pressure. The resulting crude product was diluted with water (50 mL) and extracted with ethyl acetate (3×80 mL). The combined organic layers were then washed with saturated aqueous sodium chloride solution (60 mL), dried over sodium sulfate, filtered, and concentrated under reduced pressure. The resulting crude product was purified by flash chromatography on silica gel (0-20% ethyl acetate in petroleum ether) to provide the title compound: ¹H NMR (400 MHz, CDCl₃) δ 6.69 (s, 1H), 3.87 (s, 3H), 2.33-2.25 (m, 1H), 1.22-1.17 (m, 2H), 1.07-0.96 (m, 2H).

### Step B. 6-cyclopropyl-5-methoxy-N-(4-methoxybenzyl)pyridazin-3-amine

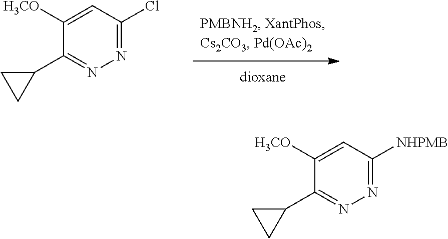

A mixture of 6-chloro-3-cyclopropyl-4-methoxy-pyridazine (4.00 g, 21.7 mmol), (4-methoxyphenyl)methanamine (8.92 g, 65.0 mmol, 8.41 mL), 4,5-bis(diphenylphosphino)-9,9-dimethyl-9H-xanthene (1.88 g, 3.25 mmol), palladium(II)acetate (0.730 g, 3.25 mmol), and cesium carbonate (21.18 g, 65.00 mmol) in dioxane (50 mL) was degassed and purged with nitrogen, and was then stirred at 120° C. for 16 hours under nitrogen atmosphere. The reaction was then cooled to room temperature, diluted with water (40 mL), and extracted with ethyl acetate (3×40 mL). The combined organic layers were washed with saturated aqueous sodium chloride solution (50 mL), dried over sodium sulfate, filtered, and concentrated under reduced pressure. The resulting crude product was purified by flash chromatography on silica gel (0-30% ethyl acetate in petroleum ether) to provide the title compound: LCMS m/z 286.2 [M+H]⁺.

### Step C. 6-cyclopropyl-5-methoxypyridazin-3-amine

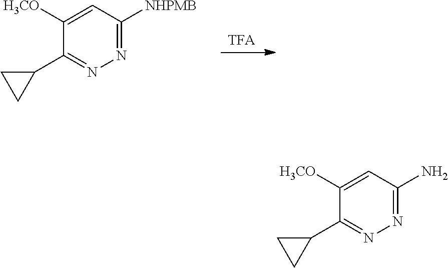

A solution of 6-cyclopropyl-5-methoxy-N-[(4-methoxyphenyl)methyl]pyridazin-3-amine (3.00 g, 10.5 mmol) in trifluoroacetic acid (30 mL) was stirred at 20° C. for 16 hours. Methanol was added, causing precipitation of a solid. The resulting mixture was filtered, and the mother liquor was concentrated under reduced pressure to provide the title compound: LCMS m/z 166.1 [M+H]⁺.

### Step D. 3-(2-bromo-5-fluoropyrimidin-4-yl)-6-cyclopropyl-7-methoxyimidazo[1,2-b]pyridazine

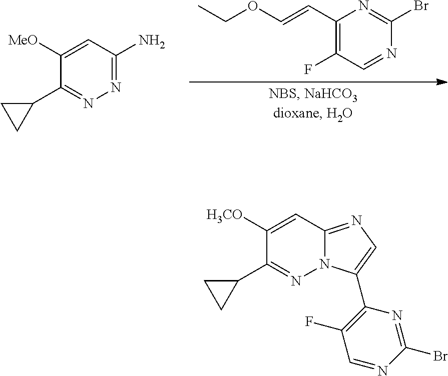

To a cooled 0° C. solution of 2-bromo-4-[(E)-2-ethoxyvinyl]-5-fluoro-pyrimidine (0.411 g, 1.66 mmol) in dioxane (9 mL) and water (3 mL) was added 1-bromopyrrolidine-2,5-dione (0.323 g, 1.82 mmol). The resulting reaction was then removed from the cold bath and stirred for 30 minutes while warming to room temperature. 6-Cyclopropyl-5-methoxy-pyridazin-3-amine (0.250 g, 1.51 mmol) and sodium bicarbonate (0.254.27 g, 3.03 mmol) were then added, and the reaction was heated to 80° C. for 2 hours. The reaction was then cooled to room temperature, diluted with water (8 mL), and extracted with ethyl acetate (3×10 mL). The organic extracts were combined, washed with saturated aqueous sodium chloride solution (8 mL), dried over sodium sulfate, filtered, and concentrated under reduced pressure. The resulting crude product was purified by flash chromatography on silica gel (0-60% ethyl acetate in petroleum ether) to provide the title compound: LCMS m/z 364.1 [M+H]⁺; ¹H NMR (400 MHz, CD₃OD) δ 8.50 (d, J=2.8 Hz, 1H), 8.11 (d, J=2.8 Hz, 1H), 7.25 (s, 1H), 3.97 (s, 3H), 2.41-2.40 (m, 1H), 1.23-1.18 (m, 2H), 1.05-0.99 (m, 2H).

### Step E. (3S,4S)-tert-butyl 3-((4-(6-cyclopropyl-7-methoxyimidazo[1,2-b]pyridazin-3-yl)-5-fluoropyrimidin-2-yl)amino)-4-fluoropiperidine-1-carboxylate

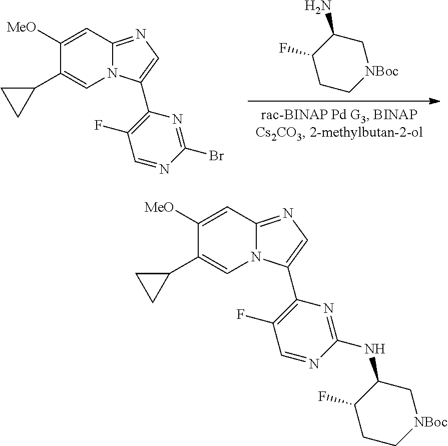

A mixture of 3-(2-bromo-5-fluoro-pyrimidin-4-yl)-6-cyclopropyl-7-methoxy-imidazo[1,2-b]pyridazine (0.025 g, 0.069 mmol), tert-butyl (3S,4S)-3-amino-4-fluoro-piperidine-1-carboxylate (0.018 g, 0.082 mmol), [2-(2-aminophenyl)phenyl]-methylsulfonyloxypalladium[1-(2-diphenylphosphanyl-1-naphthyl)-2-naphthyl]-diphenylphosphane (0.007 g, 0.007 mmol), (R)-(+)-2,2′-Bis(diphenylphosphino)-1,1′-binaphthyl (0.006 g, 0.010 mmol), and cesium carbonate (0.067 g, 0.206 mmol) in 2-methylbutan-2-ol (2 mL) was purged with nitrogen, and was then heated at 80° C. for 2 hours under nitrogen atmosphere. The reaction mixture was then cooled to room temperature, filtered, and concentrated under reduced pressure to provide the title compound: LCMS m/z 502.5 [M+H]⁺.

### Step F. 4-(6-cyclopropyl-7-methoxyimidazo[1,2-b]pyridazin-3-yl)-5-fluoro-N-((3S,4S)-4-fluoropiperidin-3-yl)pyrimidin-2-amine

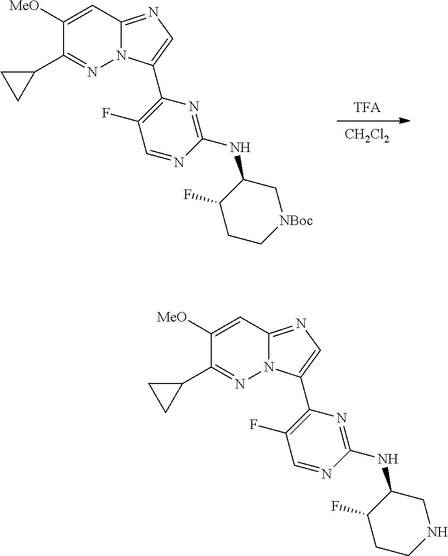 [SMILES: COc1cc2ncc(-c3nc(N[C@H]4CN(C(=O)OC(C)(C)C)CC[C@@H]4F)ncc3F)n2nc1C1CC1 ; "CH2Cl2" ; COc1cc2ncc(-c3nc(N[C@H]4CNCC[C@@H]4F)ncc3F)n2nc1C1CC1 ; "TFA"]

To a solution of tert-butyl (3S,4S)-3-[[4-(6-cyclopropyl-7-methoxy-imidazo[1,2-b]pyridazin-3-yl)-5-fluoro-pyrimidin-2-yl]amino]-4-fluoro-piperidine-1-carboxylate (0.030 g, 0.060 mmol) in dichloromethane (2 mL) was added trifluoroacetic acid (0.770 g, 6.75 mmol, 0.50 mL). The resulting reaction was stirred at room temperature for one hour, and was then concentrated under reduced pressure. The crude product thus obtained was purified by HPLC (Phenomenex Luna C18 column, 3 micron, 80×30 mm; 5-35% acetonitrile in water containing 0.1% trifluoroacetic acid) to provide the title compound: LCMS m/z 402.1 [M+H]⁺; ¹H NMR (400 MHz, CD₃OD) δ 8.41-8.40 (m, 1H), 8.20-8.17 (m, 1H), 7.52-7.46 (m, 1H), 4.84-4.72 (m, 1H), 4.36 (m, 1H), 4.06 (s, 3H), 3.51 (td, J=3.6, 12.8 Hz, 1H), 3.40-3.33 (m, 1H), 3.13-3.02 (m, 2H), 2.48-2.44 (m, 1H), 2.40-2.27 (m, 1H), 2.03-1.89 (m, 1H), 1.18-1.07 (m, 4H).

The compounds in Table 2 were all prepared using the synthetic procedures described in Example 3.

TABLE 2
Additional compounds prepared according to Example 3.

| Compound # | Structure | IUPAC Name | LCMS |
|---|---|---|---|
| 2a | 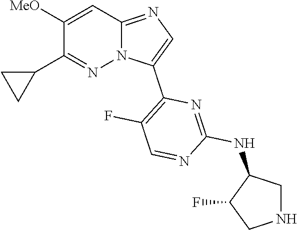 [SMILES: COc1cc2ncc(-c3nc(N[C@H]4CNC[C@@H]4F)ncc3F)n2nc1C1CC1] | 4-(6-cyclopropyl-7- methoxyimidazo[1,2- b]pyridazin-3-yl)-5- fluoro-N-((3S,4S)-4- fluoropyrrolidin-3- yl)pyrimidin-2-amine | 388.0 |
| 3a | 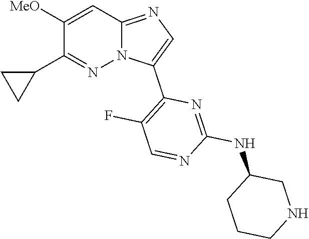 [SMILES: COc1cc2ncc(-c3nc(N[C@@H]4CCCNC4)ncc3F)n2nc1C1CC1] | (R)-4-(6-cyclopropyl-7- methoxyimidazo[1,2- b]pyridazin-3-yl)-5- fluoro-N-(piperidin-3- yl)pyrimidin-2-amine | 384.1 |

### Example 4

### Exemplary Synthetic Procedure #2 (Compounds 4a-6a)

### Compound 4a, 4-(6-cyclopropyl-7-(2,2-difluoroethoxy)imidazo[1,2-b]pyridazin-3-yl)-N-((3 S,4S)-4-fluoropiperidin-3-yl)pyrimidin-2-amine

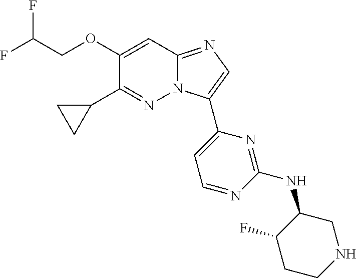 [SMILES: FC(F)COc1cc2ncc(-c3ccnc(N[C@H]4CNCC[C@@H]4F)n3)n2nc1C1CC1]

### Step A. 6-chloro-4-(2,2-difluoroethoxy)pyridazin-3-amine

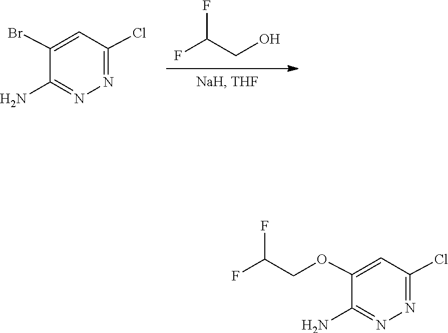

A solution of 2,2-difluoroethanol (21.65 g, 263.86 mmol) and tetrahydrofuran (300 mL) was purged with nitrogen and cooled to 0° C. Sodium hydride (14.39 g, 359.8 mmol, 60% purity) was then added in portions, and the resulting mixture was removed from the cold bath and stirred for 1 hour while warming to room temperature. 4-Bromo-6-chloro-pyridazin-3-amine (50.00 g, 239.9 mmol) was then added, and the resulting reaction was heated to 40° C. for 3 hours. The reaction was then cooled to room temperature, quenched with water (200 mL), and extracted with ethyl acetate (3×300 mL). The organic extracts were combined, washed with saturated aqueous sodium chloride solution (2×300 mL), dried over sodium sulfate, filtered, and concentrated under reduced pressure. The resulting crude product was purified by flash chromatography on silica gel (0-50% ethyl acetate in petroleum ether) to provide the title compound: LCMS m/z 210.1 M+H]⁺. ¹H NMR (400 MHz, CD₃OD) δ 7.09 (s, 1H), 6.48-6.08 (tt, J=³0.6, 54.4, 1H), 4.53-4.37 (td, J=3.6 15.0, 2H).

### Step B. 3-bromo-6-chloro-4-(2,2-difluoroethoxy)pyridazine

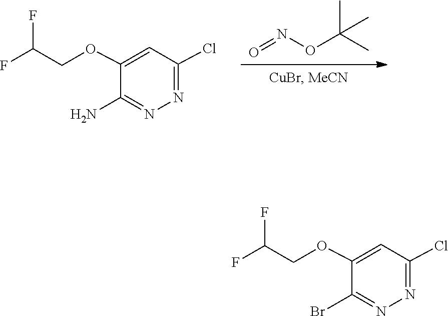

A mixture of 6-chloro-4-(2,2-difluoroethoxy)pyridazin-3-amine (31.00 g, 147.9 mmol), copper(I)bromide (27.76 g, 193.5 mmol, 5.89 mL), and acetonitrile (300 mL), was purged with nitrogen and cooled to 0° C. Tert-butyl nitrite (40.12 g, 389.0 mmol, 46.27 mL) was then added in several portions over 30 minutes. The resulting reaction was warmed to room temperature and stirred for 16 hours. The reaction was then quenched by addition of 2-[2-[bis(carboxymethyl)amino]ethyl-(carboxymethyl)amino]acetic acid (1 g), diluted with water (200 mL), and extracted with ethyl acetate (3×300 mL). The organic extracts were combined, washed with saturated aqueous sodium chloride solution (3×200 mL), dried over sodium sulfate, filtered, and concentrated under reduced pressure. The resulting crude product was purified by flash chromatography on silica gel (0-50% ethyl acetate in petroleum ether) to provide the title compound: LCMS m/z 272.9 [M+H]⁺; ¹H NMR (400 MHz, DMSO-d₆) δ 6.97 (s, 1H), 5.83-5.52 (tt, J=3.6 54.0, 1H), 3.90-3.79 (td, J=3.6 14.8, 2H).

### Step C. 6-chloro-3-cyclopropyl-4-(2,2-difluoroethoxy)pyridazine

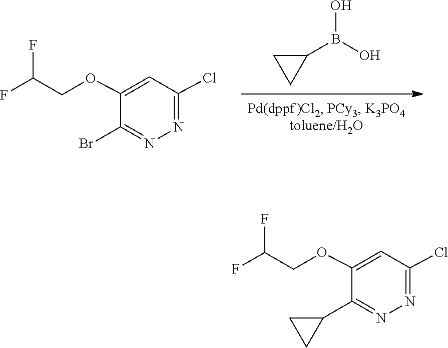 [SMILES: FC(F)COc1cc(Cl)nnc1Br ; OB(O)C1CC1 ; "Pd(dppf)Cl2, PCy3, K3PO4toluene/H2O" ; FC(F)COc1cc(Cl)nnc1C1CC1]

A 2 L three-necked flask equipped with a stirrer bar was charged with 3-bromo-6-chloro-4-(2,2-difluoroethoxy)pyridazine (19.0 g, 69.5 mmol), cyclopropylboronic acid (2.98 g, 34.7 mmol), [1,1-bis(diphenylphosphino)ferrocene]dichloropalladium(II) (5.08 g, 6.95 mmol), tricyclohexylphosphine (2.92 g, 10.4 mmol, 3.38 mL), tripotassium phosphate (2.5 M, 83.4 mL), toluene (200 mL) and water (20 mL). The resulting reaction mixture was purged with nitrogen and heated at 80° C. for 16 hours. The reaction was then cooled to room temperature and extracted with ethyl acetate (3×300 mL). The organic extracts were combined, washed with saturated aqueous sodium chloride solution (3×100 mL), dried over sodium sulfate, filtered, and concentrated under reduced pressure. The resulting crude product was purified by flash chromatography on silica gel (0-50% ethyl acetate in petroleum ether) to provide the title compound: LCMS m/z 235.1 [M+H]⁺; ¹H NMR (400 MHz, CD₃OD) δ 7.34 (s, 1H), 6.45-6.16 (tt, J=3.6 54.0, 1H), 4.53-4.43 (td, J=3.6 14.8, 2H), 2.48-2.40 (m, 1H), 1.22-1.08 (m, 4H).

### Step D. tert-butyl (6-cyclopropyl-5-(2,2-difluoroethoxy)pyridazin-3-yl)carbamate

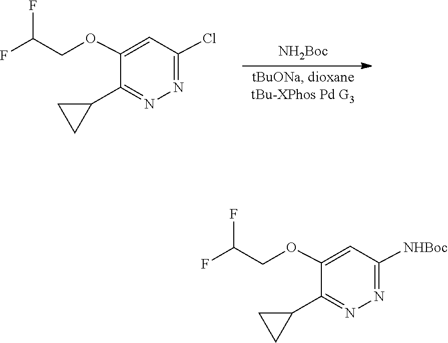 [SMILES: FC(F)COc1cc(Cl)nnc1C1CC1 ; "tBuONa, dioxanetBu-XPhos Pd G3" ; CC(C)(C)OC(=O)Nc1cc(OCC(F)F)c(C2CC2)nn1 ; "NH2Boc"]

A 2 L three-necked flask equipped with a stirrer bar was charged with 6-chloro-3-cyclopropyl-4-(2,2-difluoroethoxy)pyridazine (11.00 g, 46.88 mmol), tert-butyl carbamate (10.98 g, 93.76 mmol), [2-(2-aminophenyl)phenyl]-methylsulfonyloxy-palladium di-tert-butyl-[2-(2,4,6-triisopropylphenyl)phenyl]phosphane (3.72 g, 4.69 mmol), dioxane (100 mL), and sodium tert-butoxide solution (2 M in THF, 46.88 mL). The resulting reaction mixture was then purged with nitrogen and stirred at 100° C. for 16 hours under nitrogen atmosphere. The reaction was then cooled to room temperature, diluted with water (100 mL), and extracted with ethyl acetate (3×300 mL). The organic extracts were combined, washed with saturated aqueous sodium chloride solution (3×200 mL), dried over sodium sulfate, filtered, and concentrated under reduced pressure. The resulting crude product was purified by flash chromatography on silica gel (0-100% ethyl acetate in petroleum ether) to provide the title compound: LCMS m/z 316.1 [M+H]⁺.

### Step E. 6-cyclopropyl-5-(2,2-difluoroethoxy)pyridazin-3-amine

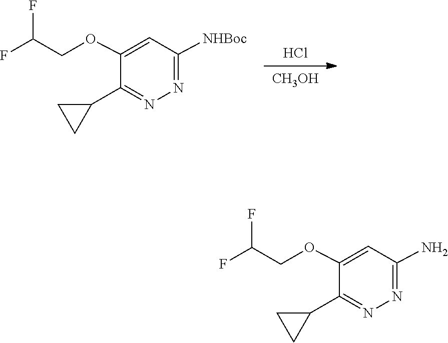

A 2 L three-necked flask equipped with a stirrer bar was charged with tert-butyl N-[6-cyclopropyl-5-(2,2-difluoroethoxy)pyridazin-3-yl]carbamate (14.00 g, 44.40 mmol) and a solution of hydrochloric acid in methanol (4 M, 140 mL). The resulting reaction was then stirred at 40° C. for 16 hours. The reaction was then cooled to room temperature and concentrated under reduced pressure to provide the title compound: LCMS m/z 216.2 [M+H]⁺.

### Step F. 6-cyclopropyl-7-(2,2-difluoroethoxy)imidazo[1,2-b]pyridazine

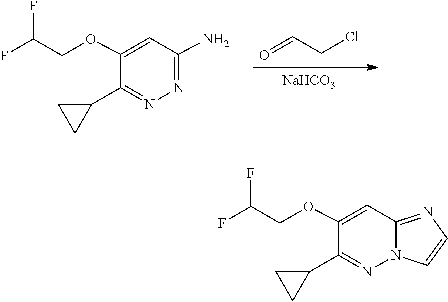

To a solution of 6-cyclopropyl-5-(2,2-difluoroethoxy)pyridazin-3-amine (6.00 g, 27.9 mmol) and 2-chloroacetaldehyde (27.36 g, 139.4 mmol, 22.42 mL, 40% purity) in ethanol (60 mL) was added sodium bicarbonate (4.68 g, 55.8 mmol). The resulting reaction mixture was heated at 80° C. for 4 hours. The reaction was then cooled to room temperature, diluted with water (50 mL), and extracted with ethyl acetate (3×50 mL). The organic extracts were combined, washed with saturated aqueous sodium chloride solution (3×30 mL), dried over sodium sulfate, filtered, and concentrated under reduced pressure. The resulting crude product was purified by flash chromatography on silica gel (0-100% ethyl acetate in petroleum ether) to provide the title compound: LCMS m/z 240.2 [M+H]⁺; ¹H NMR (400 MHz, CD₃OD) δ 7.81-7.77 (s, 1H), 7.50-7.43 (s, 1H), 7.28 (s, 1H), 6.45-6.16 (tt, J=3.6 54.0, 1H), 4.53-4.43 (td, J=3.6 14.8, 2H), 2.49-2.40 (m, 1H), 1.14-1.06 (m, 4H).

### Step G. 6-cyclopropyl-7-(2,2-difluoroethoxy)-3-iodoimidazo[1,2-b]pyridazine

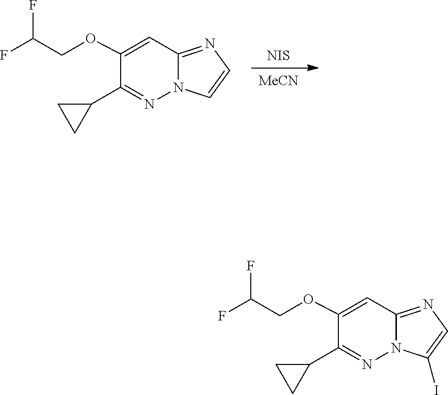

To a cooled 0° C. solution of 6-cyclopropyl-7-(2,2-difluoroethoxy)imidazo[1,2-b]pyridazine (1.00 g, 4.18 mmol) in acetonitrile (1 mL) was added 1-iodopyrrolidine-2,5-dione (1.03 g, 4.60 mmol). The resulting reaction was removed from the cold bath and stirred for 1 hour while warming to room temperature. The reaction was then concentrated under reduced pressure. The resulting crude product was triturated with ethyl acetate (3×10 mL) to provide the title compound: LCMS m/z 366 [M+H]⁺; ¹H NMR (400 MHz, CD₃OD) δ 7.54 (s, 1H), 7.32 (s, 1H), 6.49-6.15 (tt, J=3.6 54.0, 1H), 4.52-4.41 (td, J=3.6 14.8, 2H), 2.55-2.45 (m, 1H), 1.23-1.10 (m, 4H).

### Step H. 2-chloro-4-(tributylstannyl)pyrimidine

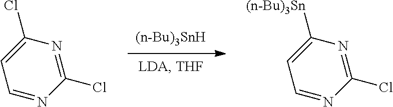

To a cooled 0° C. solution of tetrahydrofuran (400 mL) and a solution of (diisopropylamino)lithium (2 M in THF, 503.43 mL) was added tributylstannane (233.63 g, 805.49 mmol, 212.39 mL) dropwise over 30 minutes. The reaction was then cooled to −70° C. A solution of 2,4-dichloropyrimidine (100 g, 671.24 mmol) in tetrahydrofuran (600 mL) was then added dropwise. The resulting reaction was stirred at −70° C. for 4 hours, and was then warmed at room temperature and stirred for 12 hours. The reaction was then cooled to 0° C., quenched with saturated aqueous ammonium hydrochloride solution (300 mL) and saturated aqueous potassium fluoride solution (300 mL), and stirred 30 minutes while warming to room temperature. The resulting mixture was extracted with ethyl acetate (3×500 mL). The combined organic layers were washed with saturated aqueous sodium chloride solution (300 mL), dried over sodium sulfate, filtered, and concentrated under reduced pressure. The resulting crude product was purified by flash chromatography on silica gel (0-5% ethyl acetate in petroleum ether). The product thus obtained was further purified by HPLC (Phenomenex Luna C18 column, 5 micron, 250×70 mm; 60-90% acetonitrile in water containing 0.04% ammonia; carbonic acid) to provide the title compound: LCMS m/z 405.1 [M+H]⁺; ¹H NMR (400 MHz, CDCl₃) δ 8.27 (d, J=5.2 Hz, 1H), 7.28 (d, J=4.6 Hz, 1H), 1.54-1.41 (m, 6H), 1.25 (qd, J=7.3, 14.8 Hz, 6H), 1.16-1.02 (m, 6H), 0.82 (t, J=7.3 Hz, 9H).

### Step I. 3-(2-chloropyrimidin-4-yl)-6-cyclopropyl-7-(2,2-difluoroethoxy)imidazo[1,2-b]pyridazine

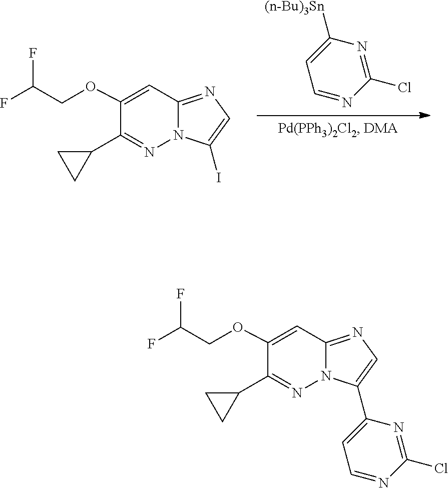

A mixture of 6-cyclopropyl-7-(2,2-difluoroethoxy)-3-iodo-imidazo[1,2-b]pyridazine (0.800 g, 2.19 mmol), tributyl-(2-chloropyrimidin-4-yl)stannane (1.33 g, 3.29 mmol), and [1,1-bis(diphenylphosphino)ferrocene]dichloropalladium(II) (0.154 g, 0.219 mmol) in N,N-dimethylacetamide (5 mL) was purged with nitrogen and heated at 100° C. for 16 hours under nitrogen atmosphere. The reaction was then cooled to room temperature, diluted with water (20 mL), and extracted with ethyl acetate (3×10 mL). The organic extracts were combined, washed with saturated aqueous sodium chloride solution (3×3 mL), dried over sodium sulfate, filtered, and concentrated under reduced pressure. The resulting crude product was purified by flash chromatography on silica gel (0-100% ethyl acetate in petroleum ether) to give the title compound: LCMS m/z 352.1 [M+H]⁺.

### Step G. (3S, 4S)-tert-butyl 3-((4-(6-cyclopropyl-7-(2,2-difluoroethoxy)imidazo[1,2-b]pyridazin-3-yl)pyrimidin-2-yl)amino)-4-fluoropiperidine-1-carboxylate

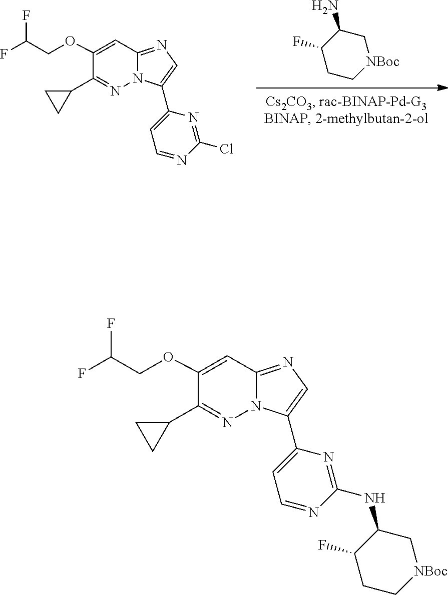

A mixture of 3-(2-chloropyrimidin-4-yl)-6-cyclopropyl-7-(2,2-difluoroethoxy)imidazo[1,2-b]pyridazine (0.050 g, 0.142 mmol), tert-butyl (3S,4S)-3-amino-4-fluoro-piperidine-1-carboxylate (0.047 g, 0.213 mmol), cesium carbonate (0.046 g, 0.142 mmol), (R)-(+)-2,2′-bis(diphenylphosphino)-1,1′-binaphthyl (0.009 g, 0.014 mmol), and [2-(2-aminophenyl)phenyl]-methylsulfonyloxy-palladium[1-(2-diphenylphosphanyl-1-naphthyl)-2-naphthyl]-diphenyl-phosphane (0.014 g, 0.014 mmol) in 2-methylbutan-2-ol (1 mL) was purged with nitrogen, and was then heated at 80° C. for 16 hours under nitrogen atmosphere. The reaction was then cooled to room temperature, filtered, and concentrated under reduced pressure. The resulting crude product was purified by prep-TLC (silica gel, 100% ethyl acetate) to give the title compound: LCMS m/z 534.3 [M+H]⁺.

### Step K. 4-(6-cyclopropyl-7-(2,2-difluoroethoxy)imidazo[1,2-b]pyridazin-3-yl)-N-((3S,4S)-4-fluoropiperidin-3-yl)pyrimidin-2-amine

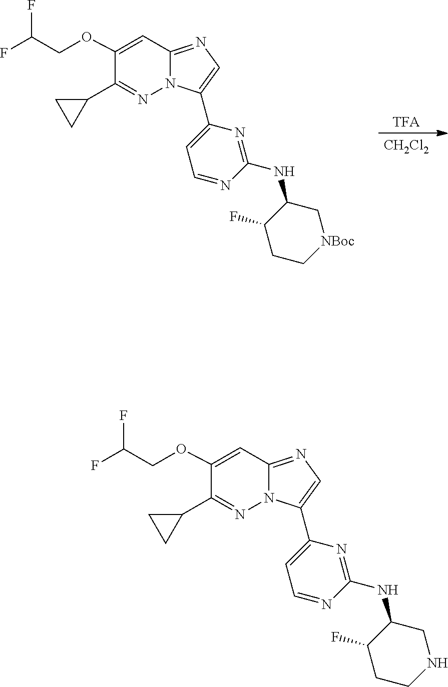 [SMILES: CC(C)(C)OC(=O)N1CC[C@H](F)[C@@H](Nc2nccc(-c3cnc4cc(OCC(F)F)c(C5CC5)nn34)n2)C1 ; "CH2Cl2" ; "TFA" ; FC(F)COc1cc2ncc(-c3ccnc(N[C@H]4CNCC[C@@H]4F)n3)n2nc1C1CC1]

To a solution of tert-butyl (3S,4S)-3-[[4-[6-cyclopropyl-7-(2,2-difluoroethoxy)imidazo[1,2-b]pyridazin-3-yl]pyrimidin-2-yl]amino]-4-fluoro-piperidine-1-carboxylate (0.050 g, 0.094 mmol) in dichloromethane (2.0 mL) was added trifluoroacetic acid (0.770 g, 6.75 mmol, 0.50 mL). The resulting reaction was stirred at room temperature for 1 hour, and was then concentrated under reduced pressure. The crude product thus obtained was purified by HPLC (Phenomenex Luna C18 column, 3 micron, 80×30 mm; 1-30% acetonitrile in water containing 0.1% hydrochloric acid) to give the title compound: LCMS m/z 434.1 [M+H]⁺. ¹H NMR (400 MHz, CD₃OD) δ 9.39 (br s, 1H), 8.65 (d, J=6.1 Hz, 1H), 8.08 (s, 1H), 7.91 (s, 1H), 6.59-6.28 (tt, J=3.6 54.0, 1H), 5.11-4.90 (m, 2H), 4.83-4.61 (td, J=3.6 14.8, 2H), 3.73 (br d, J=12.3 Hz, 1H), 3.58 (br d, J=11.6 Hz, 1H), 3.29-3.20 (m, 2H), 2.75-2.66 (m, 1H), 2.56-2.44 (m, 1H), 2.29 (br s, 1H), 1.42-1.34 (m, 4H).

The compounds in Table 3 were all prepared using the synthetic procedures described in Example 4.

TABLE 3
Additional compounds prepared according to Example 4.

| Compound # | Structure | IUPAC Name | LCMS |
|---|---|---|---|
| 5a | 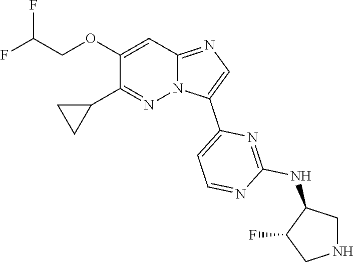 [SMILES: FC(F)COc1cc2ncc(-c3ccnc(N[C@H]4CNC[C@@H]4F)n3)n2nc1C1CC1] | 4-(6-cyclopropyl-7-(2,2- difluoroethoxy)imidazo[1,2- b]pyridazin-3-yl)-N- ((3S,4S)-4-fluoropyrrolidin- 3-yl)pyrimidin-2-amine | 420.1 |
| 6a | 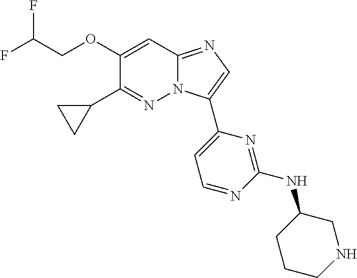 [SMILES: FC(F)COc1cc2ncc(-c3ccnc(N[C@@H]4CCCNC4)n3)n2nc1C1CC1] | (R)-4-(6-cyclopropyl-7- (2,2- difluoroethoxy)imidazo[1,2- b]pyridazin-3-yl)-N- (piperidin-3-yl)pyrimidin-2- amine | 416.1 |

### Example 5

### Exemplary Synthetic Procedure #3 (Compounds 7a-9a)

### Compound 7a, 6-(6-cyclopropyl-7-(2,2-difluoroethoxy)imidazo[1,2-b]pyridazin-3-yl)-N-((3 S,4S)-4-fluoropiperidin-3-yl)pyrazin-2-amine

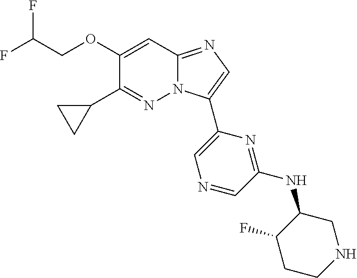 [SMILES: FC(F)COc1cc2ncc(-c3cncc(N[C@H]4CNCC[C@@H]4F)n3)n2nc1C1CC1]

### Step A. 3-(6-bromopyrazin-2-yl)-6-cyclopropyl-7-(2,2-difluoroethoxy)imidazo[1,2-b]pyridazine

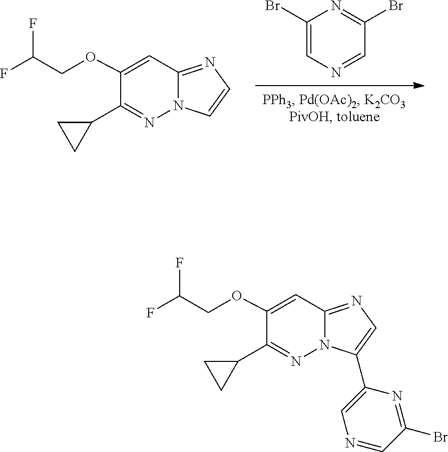

A mixture of 6-cyclopropyl-7-(2,2-difluoroethoxy)imidazo[1,2-b]pyridazine (0.500 g, 2.09 mmol), 2,6-dibromopyrazine (0.994 g, 4.18 mmol), triphenylphosphine (0.082 g, 0.314 mmol), palladium(II)acetate (0.047 g, 0.209 mmol), 2,2-dimethylpropanoic acid (0.064 g, 0.627 mmol, 72 uL), and potassium carbonate (0.867 g, 6.27 mmol) in toluene (10 mL) was purged with nitrogen, and was then heated at 100° C. for 16 hours under nitrogen atmosphere. The reaction was then cooled to room temperature, filtered, and concentrated under reduced pressure. The resulting crude product was purified by flash chromatography on silica gel (0-100% ethyl acetate in petroleum ether) to provide the title compound: LCMS m/z 396.0 [M+H]⁺.

### Step B. (3S,4S)-tert-butyl 3-((6-(6-cyclopropyl-7-(2,2-difluoroethoxy)imidazo[1,2-b]pyridazin-3-yl)pyrazin-2-yl)amino)-4-fluoropiperidine-1-carboxylate

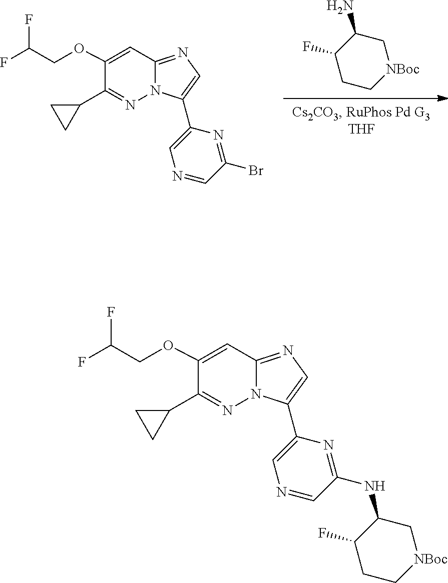

A mixture of 3-(6-bromopyrazin-2-yl)-6-cyclopropyl-7-(2,2-difluoroethoxy)imidazo[1,2-b]pyridazine (0.030 g, 0.076 mmol), tert-butyl (3S,4S)-3-amino-4-fluoro-piperidine-1-carboxylate (0.025 g, 0.114 mmol), (2-dicyclohexylphosphino-2′,6′-diisopropoxy-1,1′-bipHenyl)[2-(2′-amino-1,1′-biphenyl)]palladium(II)methanesulfonate (0.006 g, 0.008 mmol), and cesium carbonate (0.025 g, 0.076 mmol) in tetrahydrofuran (3 mL) was purged with nitrogen, and was then heated at 80° C. for 2 hours under nitrogen atmosphere. The reaction was then cooled to room temperature and concentrated under reduced pressure. The resulting crude product was purified by prep-TLC (silica gel, 10% ethyl acetate in petroleum ether) to provide the title compound: LCMS m/z 534.24 [M+H]⁺.

### Step C. 6-(6-cyclopropyl-7-(2,2-difluoroethoxy)imidazo[1,2-b]pyridazin-3-yl)-N-((3S,4S)-4-fluoropiperidin-3-yl)pyrazin-2-amine

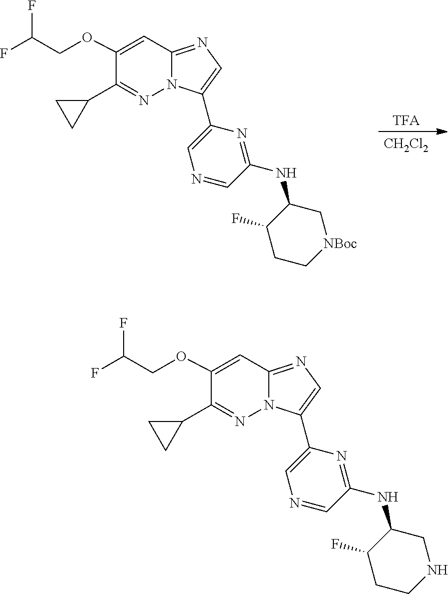 [SMILES: CC(C)(C)OC(=O)N1CC[C@H](F)[C@@H](Nc2cncc(-c3cnc4cc(OCC(F)F)c(C5CC5)nn34)n2)C1 ; "CH2Cl2" ; "TFA" ; FC(F)COc1cc2ncc(-c3cncc(N[C@H]4CNCC[C@@H]4F)n3)n2nc1C1CC1]

To a solution of tert-butyl (3S,4S)-3-[[6-[6-cyclopropyl-7-(2,2-difluoroethoxy)imidazo[1,2-b]pyridazin-3-yl]pyrazin-2-yl]amino]-4-fluoro-piperidine-1-carboxylate (0.040 g, 0.075 mmol) in dichloromethane (1.5 mL) was added trifluoroacetic acid (0.770 g, 6.75 mmol, 0.50 mL). The resulting reaction was stirred at room temperature for 2 hours, and was then concentrated under reduced pressure. The crude product thus obtained was purified by HPLC (Phenomenex Luna C18 column, 3 micron, 70×30 mm; 5-35% acetonitrile in water containing 0.1% trifluoroacetic acid) to provide the title compound: LCMS m/z 434.18 [M+H]⁺; ¹H NMR (400 MHz, CD₃OD) δ 8.91 (s, 1H), 8.41 (s, 1H), 7.98 (s, 1H), 7.61 (s, 1H), 6.53-6.24 (tt, J=3.6 54.0, 1H), 4.95 (m, 1H), 4.62-4.53 (m, 3H), 3.73 (td, J=3.5, 12.7 Hz, 1H), 3.51 (br d, J=13.1 Hz, 1H), 3.23-3.20 (m, 1H), 3.16-3.11 (m, 1H), 2.66-2.59 (m, 1H), 2.49 (m, 1H), 2.13 (m, 1H), 1.30-1.26 (m, 4H).

The compounds in Table 4 were all prepared using the synthetic procedures described in Example 5.

TABLE 4
Additional compounds prepared according to Example 5.

| Compound # | Structure | IUPAC Name | LCMS |
|---|---|---|---|
| 8a | 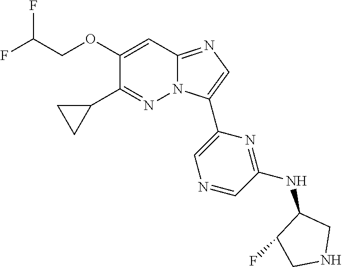 [SMILES: FC(F)COc1cc2ncc(-c3cncc(N[C@H]4CNC[C@@H]4F)n3)n2nc1C1CC1] | 6-(6-cyclopropyl-7-(2,2- difluoroethoxy)imidazo [1,2-b]pyridazin-3-yl)-N- ((3S,4S)-4- fluoropyrrolidin-3- yl)pyrazin-2-amine | 420.17 |
| 9a | 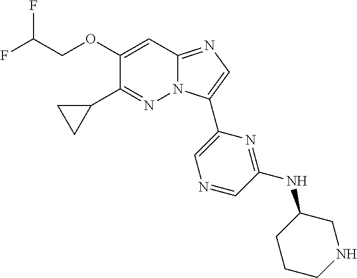 [SMILES: FC(F)COc1cc2ncc(-c3cncc(N[C@@H]4CCCNC4)n3)n2nc1C1CC1] | (R)-6-(6-cyclopropyl-7- (2,2- difluoroethoxy)imidazo [1,2-b]pyridazin-3-yl)-N- (piperidin-3-yl)pyrazin-2- amine | 416.19 |

### Example 6

### Exemplary Synthetic Procedure #4 (Compounds 10a-12a)

### Compound 10a, 4-(7-cyclopropoxy-6-cyclopropylimidazo[1,2-b]pyridazin-3-yl)-N-((3S,4S)-4-fluoropyrrolidin-3-yl)pyrimidin-2-amine

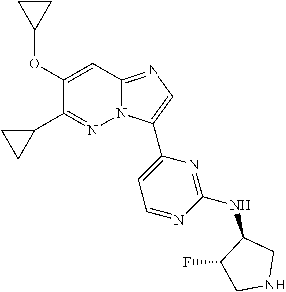 [SMILES: F[C@H]1CNC[C@@H]1Nc1nccc(-c2cnc3cc(OC4CC4)c(C4CC4)nn23)n1]

### Step A. 4-bromo-6-chloropyridazin-3-amine

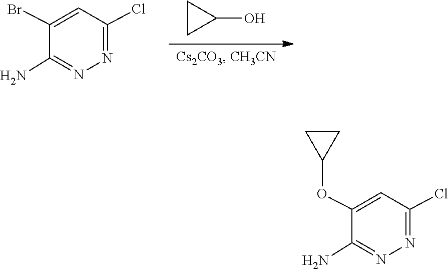

A mixture of 4-bromo-6-chloro-pyridazin-3-amine (30.00 g, 143.9 mmol), cyclopropanol (12.54 g, 215.9 mmol), and cesium carbonate (93.79 g, 287.9 mmol) in acetonitrile (1000 mL) was purged with nitrogen, and was then heated at 80° C. for 16 hours under nitrogen atmosphere. The reaction was then cooled to room temperature, quenched with water (100 mL), and extracted with ethyl acetate (3×100 mL). The organic extracts were combined, washed with saturated aqueous sodium chloride solution (3×100 mL), dried over anhydrous sodium sulfate, filtered, and concentrated under reduced pressure. The resulting crude product was purified by flash chromatography on silica gel (0-100% ethyl acetate in petroleum ether) to provide the title compound: LCMS m/z 186.00 [M+H]⁺.

### Step B. 3-bromo-6-chloro-4-cyclopropoxypyridazine

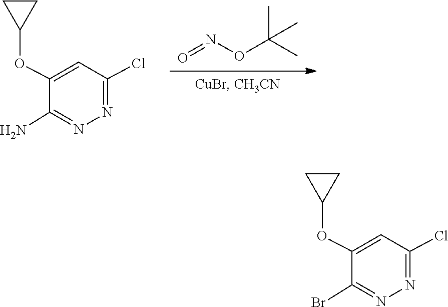

To a cooled 0° C. mixture of 6-chloro-4-(cyclopropoxy)pyridazin-3-amine (15.00 g, 80.81 mmol) and copper(I)bromide (15.19 g, 105.9 mmol) in acetonitrile (160 mL) was added dropwise tert-butyl nitrite (21.92 g, 212.5 mmol, 25.28 mL). The resulting reaction mixture was stirred at room temperature for 16 hours under nitrogen atmosphere. The reaction was then diluted with water (50 mL) and extracted with ethyl acetate (3×100 mL). The organic extracts were combined, washed with saturated aqueous sodium chloride solution (100 mL), dried over sodium sulfate, filtered, and concentrated under reduced pressure. The resulting crude product was purified by flash chromatography on silica gel (0-25% ethyl acetate in petroleum ether) to provide the title compound: LCMS m/z 250.85 [M+H]⁺; ¹HNMR (400 MHz, CD₃OD) δ 7.65 (s, 1H), 4.17-4.12 (m, 1H), 0.99-0.95 (m, 2H), 0.91-0.87 (m, 2H).

### Step C. 6-chloro-4-cyclopropoxy-3-cyclopropylpyridazine

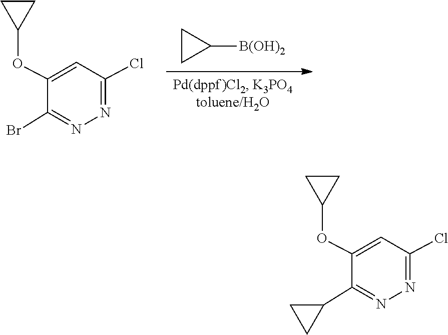 [SMILES: Clc1cc(OC2CC2)c(Br)nn1 ; OB(O)C1CC1 ; "Pd(dppf)Cl2, K3PO4toluene/H2O" ; Clc1cc(OC2CC2)c(C2CC2)nn1]

A mixture of 3-bromo-6-chloro-4-(cyclopropoxy)pyridazine (9.00 g, 36.1 mmol), cyclopropylboronic acid (3.72 g, 43.3 mmol), [1,1-bis(diphenylphosphino)ferrocene]dichloropalladium(II) (2.64 g, 3.61 mmol), and aqueous potassium phosphate solution (2.5 M, 43.29 mL) in toluene (90 mL) was purged with nitrogen, and was then heated at 100° C. for 16 hours under nitrogen atmosphere. The reaction was then cooled to room temperature, diluted with water (50 mL), and extracted with ethyl acetate (3×50 mL). The organic extracts were combined, washed with saturated aqueous sodium chloride solution (50 mL), dried over sodium sulfate, filtered, and concentrated under reduced pressure. The resulting crude product was purified by flash chromatography on silica gel (0-20% ethyl acetate in petroleum ether) to provide the title compound: LCMS m/z 211.15 [M+H]⁺; ¹HNMR (400 MHz, CD₃OD) δ 7.48 (s, 1H), 4.08-4.03 (m, 1H), 2.37-2.30 (m, 1H), 1.17-1.13 (m, 2H), 1.09-1.04 (m, 2H), 0.98-0.93 (m, 2H), 0.89-0.84 (m, 2H).

### Step D. tert-butyl (5-cyclopropoxy-6-cyclopropylpyridazin-3-yl)carbamate

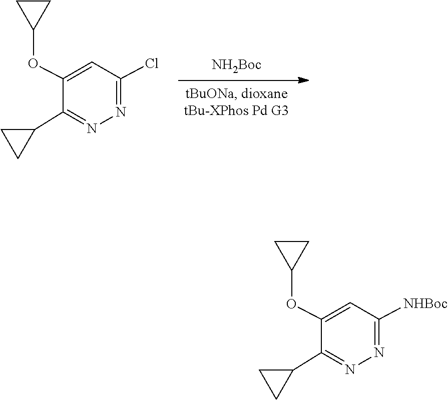 [SMILES: Clc1cc(OC2CC2)c(C2CC2)nn1 ; "tBuONa, dioxanetBu-XPhos Pd G3" ; CC(C)(C)OC(=O)Nc1cc(OC2CC2)c(C2CC2)nn1 ; "NH2Boc"]

A mixture of 6-chloro-4-(cyclopropoxy)-3-cyclopropyl-pyridazine (6.00 g, 28.5 mmol), tert-butyl carbamate (10.01 g, 85.45 mmol), sodium tert-butoxide solution (2 M in THF, 42.72 mL), and [2-(2-aminophenyl)phenyl]-methylsulfonyloxy-palladium ditert-butyl-[2-(2,4,6-triisopropylphenyl)phenyl]phosphane (1.13 g, 1.42 mmol) in dioxane (50 mL) was purged with nitrogen, and was then heated at 100° C. for 16 hours under nitrogen atmosphere. The reaction was then cooled to room temperature, diluted with water (60 mL), and extracted with ethyl acetate (3×50 mL). The organic extracts were combined, washed with saturated aqueous sodium chloride solution (50 mL), dried over sodium sulfate, filtered, and concentrated under reduced pressure. The resulting crude product was purified by flash chromatography on silica gel (0-20% ethyl acetate in petroleum ether) to provide the title compound: LCMS m/z 292.15 [M+H]⁺.

### Step E. 5-cyclopropoxy-6-cyclopropylpyridazin-3-amine

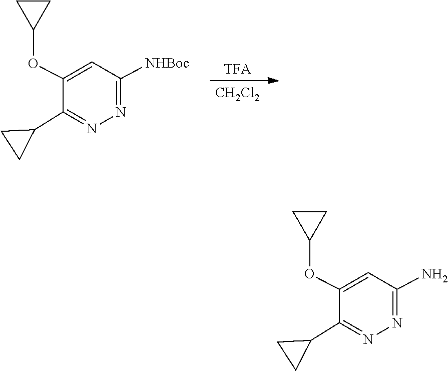 [SMILES: CC(C)(C)OC(=O)Nc1cc(OC2CC2)c(C2CC2)nn1 ; "CH2Cl2" ; Nc1cc(OC2CC2)c(C2CC2)nn1 ; "TFA"]

To a solution of tert-butyl N-[5-(cyclopropoxy)-6-cyclopropyl-pyridazin-3-yl]carbamate (1.00 g, 3.43 mmol) in dichloromethane (20 mL) was added trifluoroacetic acid (20 mL). The resulting reaction was stirred at room temperature for 1 hour, and was then concentrated under reduced pressure to provide the title compound: LCMS m/z 192.2 [M+H]⁺.

### Step F. 7-cyclopropoxy-6-cyclopropylimidazo[1,2-b]pyridazine

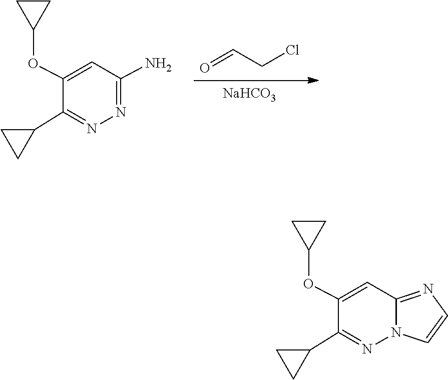

To a solution of 5-(cyclopropoxy)-6-cyclopropyl-pyridazin-3-amine (1.50 g, 4.91 mmol) in ethanol (15 mL) were added 2-chloroacetaldehyde (4.82 g, 24.6 mmol, 3.95 mL, 40% purity) and sodium bicarbonate (825.65 mg, 9.83 mmol). The resulting reaction was heated 80° C. for 4 hours. The reaction was then cooled to room temperature, diluted with water (20 mL), and extracted with ethyl acetate (3×30 mL). The organic extracts were combined, washed with saturated aqueous sodium chloride solution (30 mL), dried over sodium sulfate, filtered, and concentrated under reduced pressure. The resulting crude product was purified by flash chromatography on silica gel (0-60% ethyl acetate in petroleum ether) to provide the title compound: LCMS m/z 216.00 [M+H]⁻; ¹HNMR (400 MHz, CD₃OD) δ 8.09 (d, J=1.9 Hz, 1H), 7.86 (d, J=2.3 Hz, 1H), 7.82-7.79 (s, 1H), 4.20-4.15 (m, 1H), 2.52-2.43 (m, 1H), 1.23-1.16 (m, 4H), 1.07-1.00 (m, 2H), 1.00-0.93 (m, 2H).

### Step G. 7-cyclopropoxy-6-cyclopropyl-3-iodoimidazo[1,2-b]pyridazine

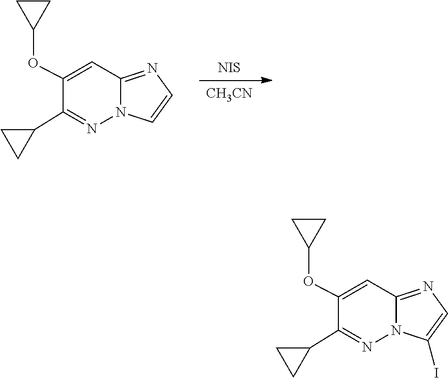

To a cooled 0° C. solution of 7-(cyclopropoxy)-6-cyclopropyl-imidazo[1,2-b]pyridazine (0.700 g, 3.25 mmol) in acetonitrile (8 mL) was added 1-iodopyrrolidine-2,5-dione (804.82 mg, 3.58 mmol). The resulting reaction was stirred for 4 hours while slowly warming to room temperature. The reaction was then diluted with water (10 mL), basified to pH ~9 by addition of aqueous sodium hydroxide solution (2 M, 2 mL), and extracted with ethyl acetate (3×10 mL). The organic extracts were combined, washed with saturated aqueous sodium chloride solution (10 mL), dried over sodium sulfate, filtered, and concentrated under reduced pressure to provide the title compound: LCMS m/z 342.00 [M+H]⁺; ¹HNMR (400 MHz, CD₃OD) δ 7.52 (s, 1H), 7.48 (s, 1H), 4.03-4.00 (m, 1H), 2.70-2.30 (m, 1H), 1.17-1.14 (m, 2H), 1.10-1.06 (m, 2H), 0.72-0.95 (m, 2H), 0.88-0.85 (m, 2H).

### Step H. 3-(2-chloropyrimidin-4-yl)-7-cyclopropoxy-6-cyclopropylimidazo[1,2-b]pyridazine

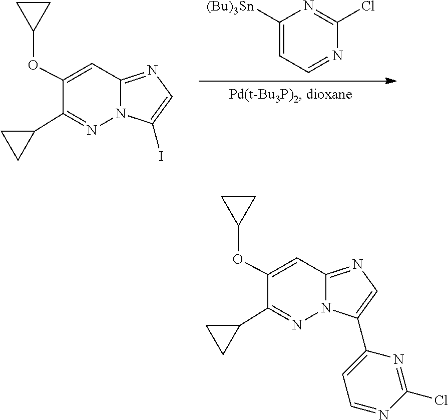

A mixture of 7-(cyclopropoxy)-6-cyclopropyl-3-iodo-imidazo[1,2-b]pyridazine PGP (0.500 g, 1.47 mmol), tributyl-(2-chloropyrimidin-4-yl)stannane (1.18 g, 2.93 mmol), and di-(tert-butylphosphine)palladium (0.075 g, 0.147 mmol) in dioxane (4 mL) was purged with nitrogen, and was then heated at 100° C. for 16 hours under nitrogen atmosphere. The reaction was then cooled to room temperature, diluted with water (30 mL), and extracted with ethyl acetate (3×30 mL). The organic extracts were combined, washed with saturated aqueous sodium chloride solution (30 mL), dried over sodium sulfate, filtered, and concentrated under reduced pressure. The resulting crude product was purified by flash chromatography on silica gel (0-40% ethyl acetate in petroleum ether) to provide the title compound: LCMS m/z 328.10 [M+H]⁺.

### Step I. tert-butyl (3S,4S)-3-((4-(7-cyclopropoxy-6-cyclopropylimidazo[1,2-b]pyridazin-3-yl)pyrimidin-2-yl)amino)-4-fluoropyrrolidine-1-carboxylate

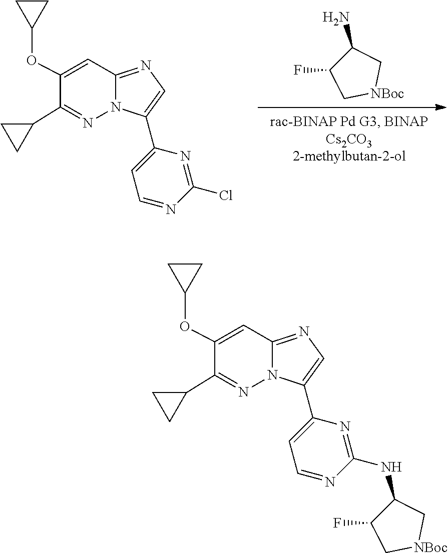

A mixture of 3-(2-chloropyrimidin-4-yl)-7-(cyclopropoxy)-6-cyclopropyl-imidazo[1,2-b]pyridazine (0.040 g, 0.122 mmol), tert-butyl (3S,4S)-3-amino-4-fluoro-pyrrolidine-1-carboxylate (0.037 g, 0.183 mmol), methanesulfonato[2,2-bis(diphenylphosphino)-1,1-binaphthyl](2-amino-1,1-biphenyl-2-yl)palladium(II) (0.012 g, 0.012 mmol), (R)-(+)-2,2′-bis(diphenylphosphino)-1,1′-binaphthyl (0.011 g, 0.018 mmol), and cesium carbonate (0.099 g, 0.305 mmol) in 2-methylbutan-2-ol (2.5 mL) was purged with nitrogen, and was then heated at 80° C. for 4 hours under nitrogen atmosphere. The reaction was then cooled to room temperature, filtered, and concentrated under reduced pressure to provide the title compound: LCMS m/z 496.25 [M+H]⁻.

### Step J. 4-(7-cyclopropoxy-6-cyclopropylimidazo[1,2-b]pyridazin-3-yl)-N-((3S,4S)-4-fluoropyrrolidin-3-yl)pyrimidin-2-amine

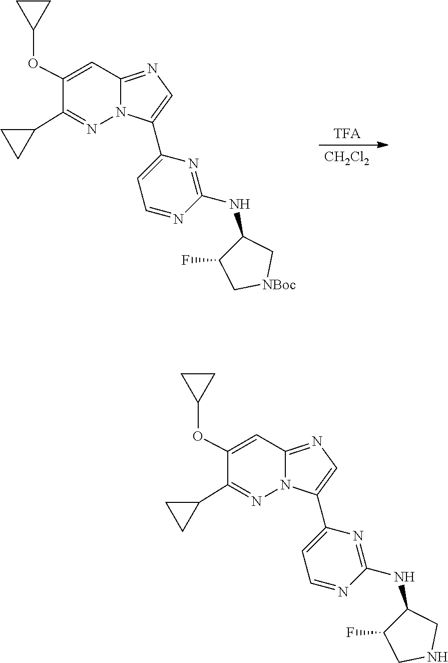 [SMILES: CC(C)(C)OC(=O)N1C[C@H](Nc2nccc(-c3cnc4cc(OC5CC5)c(C5CC5)nn34)n2)[C@@H](F)C1 ; "CH2Cl2" ; "TFA" ; F[C@H]1CNC[C@@H]1Nc1nccc(-c2cnc3cc(OC4CC4)c(C4CC4)nn23)n1]

To a solution of tert-butyl (3S,4S)-3-[[4-[7-(cyclopropoxy)-6-cyclopropyl-imidazo[1,2-b]pyridazin-3-yl]pyrimidin-2-yl]amino]-4-fluoro-pyrrolidine-1-carboxylate (0.068 g, 0.137 mmol) in dichloromethane (2 mL) was added trifluoroacetic acid (0.770 g, 6.75 mmol, 0.50 mL). The resulting reaction was stirred at room temperature for 1 hour, and was then concentrated under reduced pressure. The crude product thus obtained was purified by HPLC (Phenomenex Luna C18 column, 3 micron, 100×40 mm; 10-40% acetonitrile in water containing 0.1% trifluoroacetic acid) to provide the title compound: LCMS m/z 396.0 [M+H]⁺; ¹HNMR (400 MHz, CD₃OD) δ 8.54 (d, J=5.4 Hz, 1H), 8.51 (s, 1H), 7.85-7.84 (m, 2H), 5.54-5.38 (d, J=49.6, 1H), 4.81-4.77 (m, 1H), 4.20-4.17 (m, 1H), 3.90-3.60 (m, 4H), 2.57-2.51 (m, 1H), 1.31-1.23 (m, 4H), 1.05-0.97 (m, 4H).

The compounds in Table 5 were all prepared using the synthetic procedures described in Example 6.

TABLE 5
Additional compounds prepared according to Example 6.

| Compound # | Structure | IUPAC Name | LCMS |
|---|---|---|---|
| 11a | 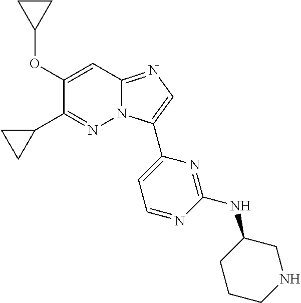 [SMILES: c1cc(-c2cnc3cc(OC4CC4)c(C4CC4)nn23)nc(N[C@@H]2CCCNC2)n1] | (R)-4-(7-cyclopropoxy- 6- cyclopropylimidazo[1,2- b]pyridazin-3-yl)-N- (piperidin-3- yl)pyrimidin-2-amine | 392.1 |
| 12a | 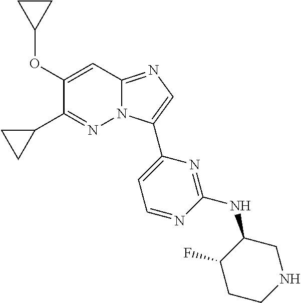 [SMILES: F[C@H]1CCNC[C@@H]1Nc1nccc(-c2cnc3cc(OC4CC4)c(C4CC4)nn23)n1] | 4-(7-cyclopropoxy-6- cyclopropylimidazo[1,2- b]pyridazin-3-yl)-N- ((3S,4S)-4- fluoropiperidin-3- yl)pyrimidin-2-amine | 410.1 |

### Example 7

### Exemplary Synthetic Procedure #5 (Compounds 13a-15a)

### Compound 13a, 6-(7-cyclopropoxy-6-cyclopropylimidazo[1,2-b]pyridazin-3-yl)-N-((3S,4S)-4-fluoropiperidin-3-yl)pyrazin-2-amine

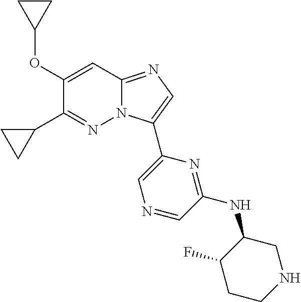 [SMILES: F[C@H]1CCNC[C@@H]1Nc1cncc(-c2cnc3cc(OC4CC4)c(C4CC4)nn23)n1]

### Step A. 3-(6-bromopyrazin-2-yl)-7-cyclopropoxy-6-cyclopropylimidazo[1,2-b]pyridazine

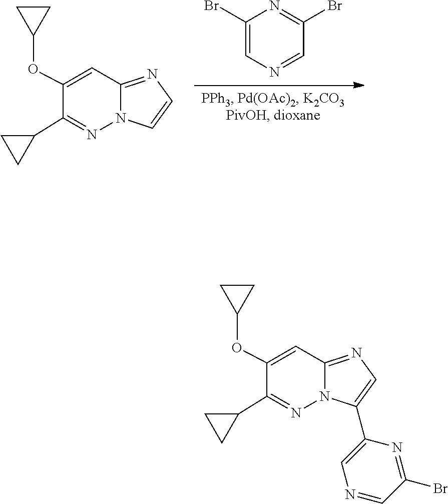

A mixture of 7-(cyclopropoxy)-6-cyclopropyl-imidazo[1,2-b]pyridazine (0.290 g, 1.35 mmol), 2,6-dibromopyrazine (0.641 g, 2.69 mmol), triphenylphosphine (0.053 g, 0.202 mmol), palladium(II)acetate (0.030 g, 0.135 mmol), 2,2-dimethylpropanoic acid (0.206 g, 2.02 mmol, 0.232 mL), and potassium carbonate (0.559 g, 4.04 mmol) in dioxane (10 mL) was purged with argon, and was then heated at 100° C. for 16 hours under argon atmosphere. The reaction was then cooled to the room temperature, diluted with water (40 mL), and extracted with ethyl acetate (3×40 mL). The organic extracts were combined, washed with saturated aqueous sodium chloride solution (40 mL), dried over sodium sulfate, filtered, and concentrated under reduced pressure. The resulting crude product was purified by flash chromatography on silica gel (0-90% ethyl acetate in petroleum ether) to provide the title compound: LCMS m/z 372.0 [M+H]⁺.

### Step B. tert-butyl (3S,4S)-3-((6-(7-cyclopropoxy-6-cyclopropylimidazo[1,2-b]pyridazin-3-yl)pyrazin-2-yl)amino)-4-fluoropiperidine-1-carboxylate

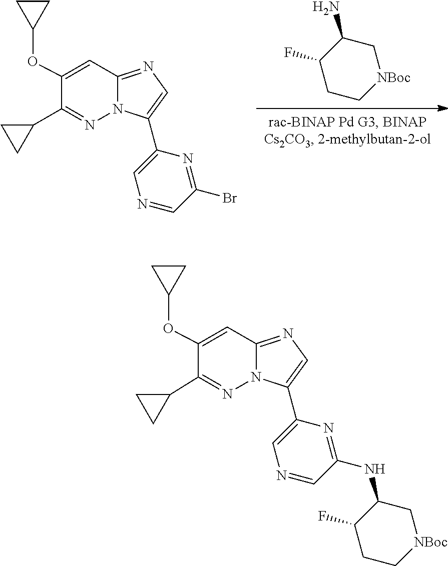

A mixture of 3-(6-bromopyrazin-2-yl)-7-(cyclopropoxy)-6-cyclopropyl-imidazo[1,2-b]pyridazine (0.025 g, 0.067 mmol), tert-butyl (3S,4S)-3-amino-4-fluoro-piperidine-1-carboxylate (0.022 g, 0.101 mmol), [2-(2-aminophenyl)phenyl]-methylsulfonyloxy-palladium[1-(2-diphenylphosphanyl-1-naphthyl)-2-naphthyl]-diphenyl-phosphane (0.007 g, 0.007 mmol), (R)-(+)-2,2′-bis(diphenylphosphino)-1,1′-binaphthyl (0.006 g, 0.010 mmol), and cesium carbonate (0.066 g, 0.201 mmol) in 2-methylbutan-2-ol (2 mL) was purged with nitrogen, and was then heated at 80° C. for 2 hours under nitrogen atmosphere. The reaction was then cooled to the room temperature, filtered, and concentrated under reduced pressure to provide the title compound: LCMS m/z 510.26 [M+H]⁻.

### Step C. 6-(7-cyclopropoxy-6-cyclopropylimidazo[1,2-b]pyridazin-3-yl)-N-((3S,4S)-4-fluoropiperidin-3-yl)pyrazin-2-amine

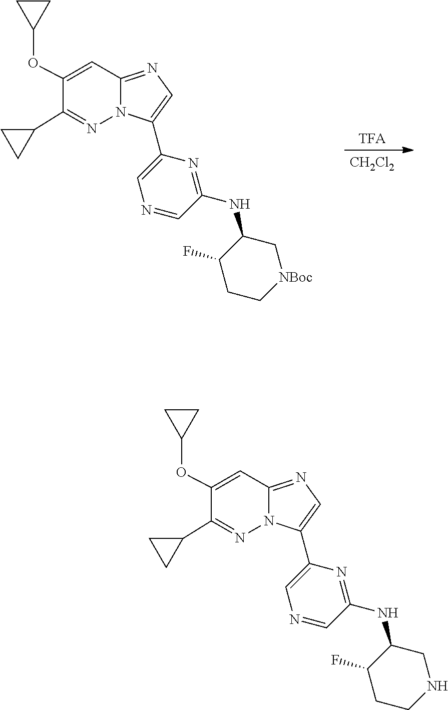 [SMILES: CC(C)(C)OC(=O)N1CC[C@H](F)[C@@H](Nc2cncc(-c3cnc4cc(OC5CC5)c(C5CC5)nn34)n2)C1 ; "CH2Cl2" ; "TFA" ; F[C@H]1CCNC[C@@H]1Nc1cncc(-c2cnc3cc(OC4CC4)c(C4CC4)nn23)n1]

To a solution of tert-butyl (3S,4S)-3-[[6-[7-(cyclopropoxy)-6-cyclopropyl-imidazo[1,2-b]pyridazin-3-yl]pyrazin-2-yl]amino]-4-fluoro-piperidine-1-carboxylate (0.050 g, 0.098 mmol) in dichloromethane (1.5 mL) was added trifluoroacetic acid (0.770 g, 6.75 mmol, 0.50 mL). The resulting reaction was stirred at room temperature for 1 hour, and was then concentrated under reduced pressure. The crude product thus obtained was purified by HPLC (Phenomenex Luna C18 column, 3 micron, 150×30 mm; 1-40% acetonitrile in water containing 0.1% trifluoroacetic acid) to provide the title compound: LCMS m/z 410.20 [M+H]⁻; ¹H NMR (400 MHz, CD₃OD) δ 8.90 (s, 1H), 8.46 (s, 1H), 8.00 (s, 1H), 7.81 (s, 1H), 4.95 (br dd, J=4.4, 8.5 Hz, 1H), 4.54 (dq, J=4.4, 8.8 Hz, 1H), 4.18 (tt, J=2.9, 5.9 Hz, 1H), 3.73 (td, J=3.6, 12.9 Hz, 1H), 3.56-3.48 (m, 1H), 3.28-3.19 (m, 1H), 3.13 (dd, J=9.1, 12.8 Hz, 1H), 2.61-2.39 (m, 2H), 2.20-2.07 (m, 1H), 1.30-1.22 (m, 4H), 1.08-0.94 (m, 4H).

The compounds in Table 6 were all prepared using the synthetic procedures described in Example 7.

TABLE 6
Additional compounds prepared according to Example 7.

| Compound # | Structure | IUPAC Name | LCMS |
|---|---|---|---|
| 14a | 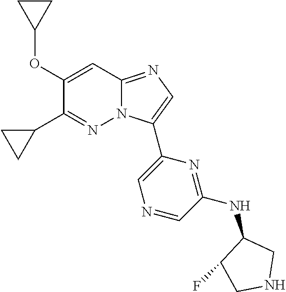 [SMILES: F[C@H]1CNC[C@@H]1Nc1cncc(-c2cnc3cc(OC4CC4)c(C4CC4)nn23)n1] | 6-(7-cyclopropoxy-6- cyclopropylimidazo[1,2- b]pyridazin-3-yl)-N- ((3S,4S)-4- fluoropyrrolidin-3- yl)pyrazin-2-amine | 396.1 |
| 15a | 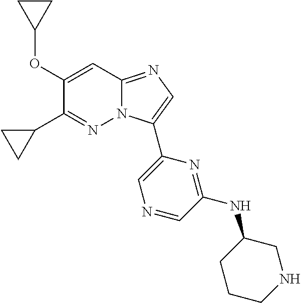 [SMILES: c1ncc(-c2cnc3cc(OC4CC4)c(C4CC4)nn23)nc1N[C@@H]1CCCNC1] | (R)-6-(7-cyclopropoxy-6- cyclopropylimidazo[1,2- b]pyridazin-3-yl)-N- (piperidin-3-yl)pyrazin-2- amine | 392.2 |

### Example 8

### Exemplary Synthetic Procedure #6 (Compounds 16a-18a)

### Compound 16a, 4-(6-(3,3-difluoroazetidin-1-yl)-7-methoxyimidazo[1,2-b]pyridazin-3-yl)-N-((3 S,4S)-4-fluoropyrrolidin-3-yl)pyrimidin-2-amine

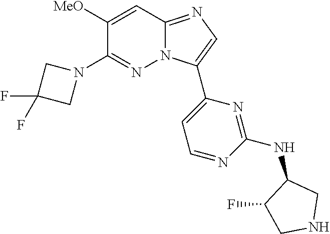 [SMILES: COc1cc2ncc(-c3ccnc(N[C@H]4CNC[C@@H]4F)n3)n2nc1N1CC(F)(F)C1]

### Step A. 6-chloro-3-(3,3-difluoroazetidin-1-yl)-4-methoxypyridazine

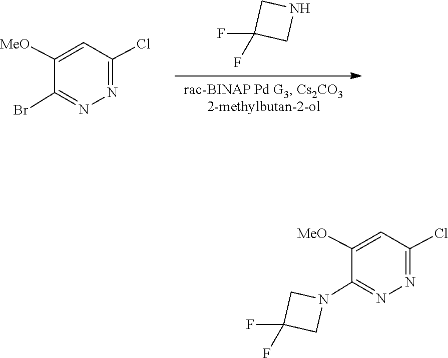

A mixture of 3-bromo-6-chloro-4-methoxy-pyridazine (4.40 g, 19.7 mmol), 3,3-difluoroazetidine (5.10 g, 39.4 mmol), [2-(2-aminophenyl)phenyl]-methylsulfonyloxypalladium[1-(2-diphenylphosphanyl-1-naphthyl)-2-naphthyl]-diphenylphosphane (0.977 g, 0.985 mmol), and cesium carbonate (12.83 g, 39.38 mmol) in 2-methylbutan-2-ol (3 mL) was purged with nitrogen, and was then heated at 90° C. for 16 hours under nitrogen atmosphere. The reaction was then cooled to room temperature, filtered, and concentrated under reduced pressure. The resulting crude product was purified by flash chromatography on silica gel (0-100% ethyl acetate in petroleum ether) to provide the title compound: LCMS m/z 235.9 [M+H]⁺.

### Step B. tert-butyl (6-(3,3-difluoroazetidin-1-yl)-5-methoxypyridazin-3-yl)carbamate

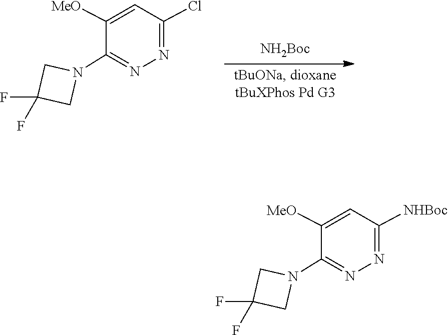 [SMILES: COc1cc(Cl)nnc1N1CC(F)(F)C1 ; "tBuONa, dioxanetBuXPhos Pd G3" ; COc1cc(NC(=O)OC(C)(C)C)nnc1N1CC(F)(F)C1 ; "NH2Boc"]

A mixture of 6-chloro-3-(3,3-difluoroazetidin-1-yl)-4-methoxypyridazine (1.50 g, 6.37 mmol), tert-butyl carbamate (1.49 g, 12.7 mmol), sodium tert-butoxide solution (2 M in THF, 6.37 mL), and [2-(2-aminophenyl)phenyl]-methylsulfonyloxypalladiumditert-butyl-[2-(2,4,6-triisopropylphenyl)phenyl]phosphane (0.506 g, 0.637 mmol) in dioxane (20 mL) was purged with nitrogen, and was then heated at 100° C. for 16 hours under nitrogen atmosphere. The reaction was then cooled to room temperature, diluted with water (40 mL), and extracted with ethyl acetate (3×30 mL). The organic extracts were combined, washed with saturated aqueous sodium chloride solution (3×20 mL), dried over sodium sulfate, filtered, and concentrated under reduced pressure. The resulting crude product was purified by flash chromatography on silica gel (0-100% ethyl acetate in petroleum ether) to provide the title compound LCMS m/z 317.2 [M+H]⁺.

### Step C. 6-(3,3-difluoroazetidin-1-yl)-5-methoxypyridazin-3-amine

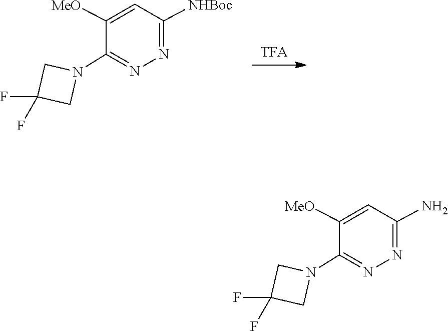 [SMILES: COc1cc(NC(=O)OC(C)(C)C)nnc1N1CC(F)(F)C1 ; COc1cc(N)nnc1N1CC(F)(F)C1 ; "TFA"]

A mixture of tert-butyl N-[6-(3,3-difluoroazetidin-1-yl)-5-methoxy-pyridazin-3-yl]carbamate (1.00 g, 3.16 mmol) in trifluoroacetic acid (10 mL) was stirred at 0° C. for 30 minutes. The reaction was then concentrated under reduced pressure to provide the title compound: LCMS m/z 217.1 [M+H]⁺.

### Step D. 6-(3,3-difluoroazetidin-1-yl)-7-methoxyimidazo[1,2-b]pyridazine

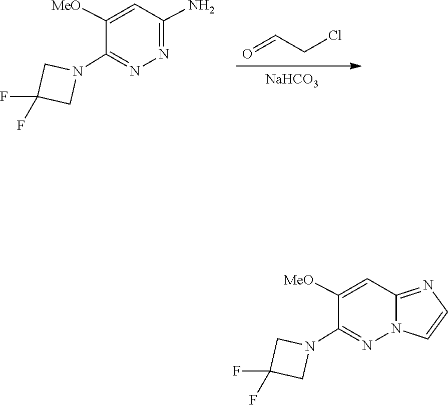

To a solution of 6-(3,3-difluoroazetidin-1-yl)-5-methoxy-pyridazin-3-amine (0.600 g, 2.78 mmol) and 2-chloroacetaldehyde (2.72 g, 13.9 mmol, 2.23 mL, 40% purity) in ethanol (20 mL) was added sodium bicarbonate (0.466 g, 5.55 mmol). The resulting reaction was heated at 80° C. for 4 hours. The reaction was then cooled to room temperature, diluted with water (40 mL), and extracted with ethyl acetate (3×30 mL). The organic extracts were combined, washed with saturated aqueous sodium chloride solution (3×20 mL), dried over sodium sulfate, filtered, and concentrated under reduced pressure. The resulting crude product was purified by flash chromatography on silica gel (0-100% ethyl acetate in petroleum ether) to provide the title compound LCMS m/z 241.1 [M+H]⁺.

### Step E. 6-(3,3-difluoroazetidin-1-yl)-3-iodo-7-methoxyimidazo[1,2-b]pyridazine

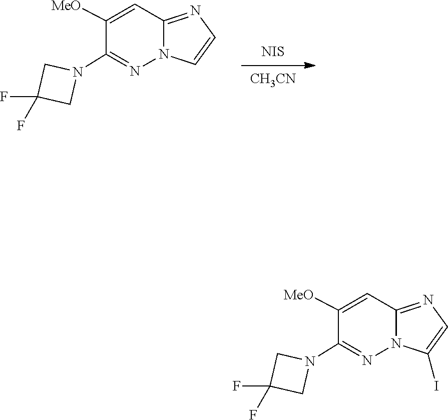

To a cooled 0° C. solution of 6-(3,3-difluoroazetidin-1-yl)-7-methoxy-imidazo[1,2-b]pyridazine (0.380 g, 1.58 mmol) in acetonitrile (4 mL) was added 1-iodopyrrolidine-2,5-dione (391.50 mg, 1.74 mmol). The resulting reaction was stirred for 1 hour while warming to room temperature, and was then filtered and concentrated under reduced pressure. The crude product thus obtained was triturated with ethyl acetate (3×5 mL) to provide the title compound: LCMS m/z 367.0 [M+H]⁺; ¹H NMR (400 MHz, CD₃OD) δ 7.39 (s, 1H), 7.11 (s, 1H), 4.56 (t, J=12.3 Hz, 4H), 3.96 (s, 3H).

### Step F. 3-(2-chloropyrimidin-4-yl)-6-(3,3-difluoroazetidin-1-yl)-7-methoxyimidazo[1,2-b]pyridazine

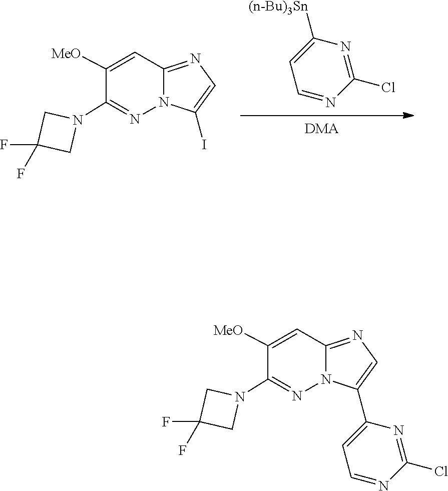

A mixture of tributyl-(2-chloropyrimidin-4-yl)stannane (0.562 g, 1.39 mmol), 6-(3,3-difluoroazetidin-1-yl)-3-iodo-7-methoxy-imidazo[1,2-b]pyridazine (0.340 g, 0.929 mmol), and [1,1-bis(diphenylphosphino)ferrocene]dichloropalladium(II) (0.065 g, 0.093 mmol) in N,N-dimethylacetamide (8 mL) was purged with nitrogen, and was then heated at 100° C. for 16 hours under nitrogen atmosphere. The reaction was then cooled to room temperature, quenched with saturated aqueous ammonium chloride solution (10 mL) and saturated aqueous potassium fluoride solution (10 mL), and extracted with ethyl acetate (3×10 mL). The organic extracts were combined, washed with saturated aqueous sodium chloride solution (10 mL), dried over sodium sulfate, filtered, and concentrated under reduced pressure. The resulting crude product was purified by flash chromatography on silica gel (0-10% methanol in dichloromethane) to provide the title compound: LCMS m/z 352.9 [M+H]⁺.

### Step G. (3S,4S)-tert-butyl 3-((4-(6-(3,3-difluoroazetidin-1-yl)-7-methoxyimidazo[1,2-b]pyridazin-3-yl)pyrimidin-2-yl)amino)-4-fluoropyrrolidine-1-carboxylate

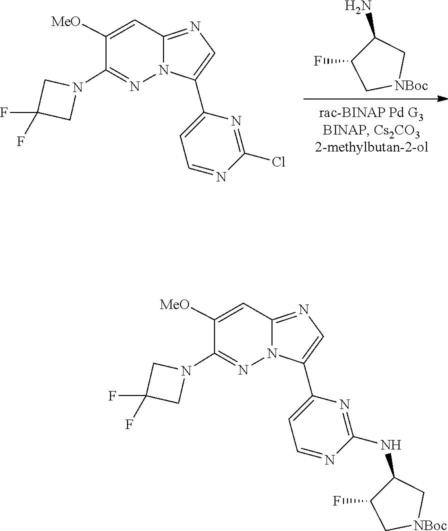

A mixture of 3-(2-chloropyrimidin-4-yl)-6-(3,3-difluoroazetidin-1-yl)-7-methoxy-imidazo[1,2-b]pyridazine (0.045 g, 0.128 mmol), tert-butyl (3S,4S)-3-amino-4-fluoro-pyrrolidine-1-carboxylate (0.031 g, 0.153 mmol), cesium carbonate (0.104 g, 0.319 mmol), [2-(2-aminophenyl)phenyl]-methylsulfonyloxypalladium[1-(2-diphenylphosphanyl-1-naphthyl)-2-naphthyl]-diphenylphosphane (0.013 g, 0.013 mmol), and (R)-(+)-2,2′-bis(diphenylphosphino)-1,1′-binaphthyl (0.012 g, 0.019 mmol) in 2-methylbutan-2-ol (1 mL) was purged with nitrogen, and was then heated at 80° C. for 6 hours under nitrogen atmosphere. The reaction was then cooled to room temperature, filtered, and concentrated under reduced pressure to provide the title compound: LCMS m/z 521.5 [M+H]⁺.

### Step H. 4-(6-(3,3-difluoroazetidin-1-yl)-7-methoxyimidazo[1,2-b]pyridazin-3-yl)-N-((3S,4S)-4-fluoropyrrolidin-3-yl)pyrimidin-2-amine

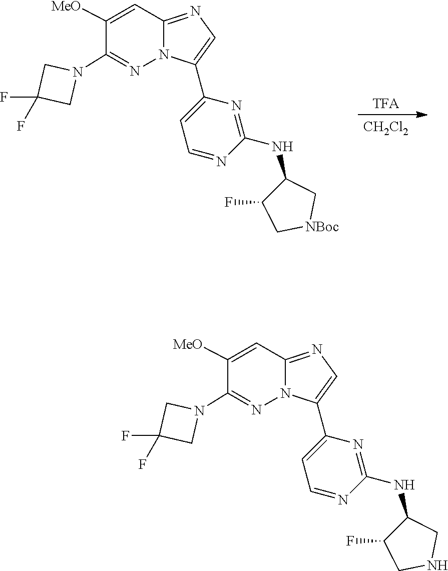 [SMILES: COc1cc2ncc(-c3ccnc(N[C@H]4CN(C(=O)OC(C)(C)C)C[C@@H]4F)n3)n2nc1N1CC(F)(F)C1 ; "CH2Cl2" ; "TFA" ; COc1cc2ncc(-c3ccnc(N[C@H]4CNC[C@@H]4F)n3)n2nc1N1CC(F)(F)C1]

To a solution of tert-butyl (3S,4S)-3-[[4-[6-(3,3-difluoroazetidin-1-yl)-7-methoxy-imidazo[1,2-b]pyridazin-3-yl]pyrimidin-2-yl]amino]-4-fluoro-pyrrolidine-1-carboxylate (0.050 g, 0.096 mmol) in dichloromethane (1 mL) was added trifluoroacetic acid (0.50 mL). The resulting reaction was stirred at room temperature for 1 hour, and was then concentrated under reduced pressure. The crude product thus obtained was purified by HPLC (Phenomenex Luna C18 column, 3 micron, 75×30 mm; 1-30% acetonitrile in water containing 0.1% trifluoroacetic acid) to provide the title compound: LCMS m/z 421.1 [M+H]⁺; ¹H NMR (400 MHz, CD₃OD) δ 8.53 (d, J=5.3 Hz, 1H), 8.38 (br s, 1H), 7.95 (d, J=5.4 Hz, 1H), 7.42 (s, 1H), 5.54-5.38 (d, J=50.4 Hz, 1H), 4.73 (m, 5H), 4.08 (s, 3H), 3.91-3.74 (m, 2H), 3.72-3.57 (m, 2H).

The compounds in Table 7 were all prepared using the synthetic procedures described in Example 8.

TABLE 7
Additional compounds prepared according to Example 8.

| Compound # | Structure | IUPAC Name | LCMS |
|---|---|---|---|
| 17a | 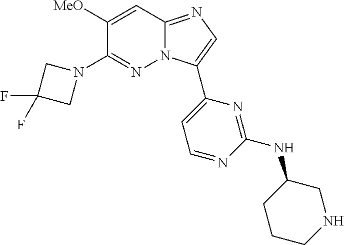 [SMILES: COc1cc2ncc(-c3ccnc(N[C@@H]4CCCNC4)n3)n2nc1N1CC(F)(F)C1] | (R)-4-(6-(3,3- difluoroazetidin-1-yl)-7- methoxyimidazo[1,2- b]pyridazin-3-yl)-N- (piperidin-3-yl)pyrimidin- 2-amine | 417.1 |
| 18a | 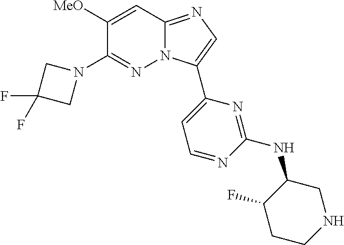 [SMILES: COc1cc2ncc(-c3ccnc(N[C@H]4CNCC[C@@H]4F)n3)n2nc1N1CC(F)(F)C1] | 4-(6-(3,3-difluoroazetidin- 1-yl)-7- methoxyimidazo[1,2- b]pyridazin-3-yl)-N- ((3S,4S)-4-fluoropiperidin- 3-yl)pyrimidin-2-amine | 434.1 |

### Example 9

### Exemplary Synthetic Procedure #7 (Compounds 19a-21a)

### Compound 19a, 4-(6-(3,3-difluorocyclobutyl)-7-methoxyimidazo[1,2-b]pyridazin-3-yl)-N-((3 S,4S)-4-fluoropiperidin-3-yl)pyrimidin-2-amine

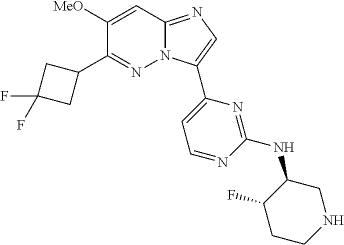 [SMILES: COc1cc2ncc(-c3ccnc(N[C@H]4CNCC[C@@H]4F)n3)n2nc1C1CC(F)(F)C1]

### Step A. 6-chloro-3-(3,3-difluorocyclobutyl)-4-methoxypyridazine

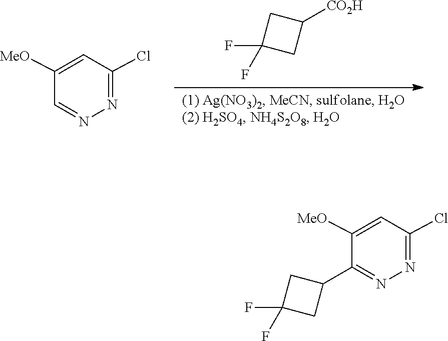 [SMILES: COc1cnnc(Cl)c1 ; "(1) Ag(NO3)2, MeCN, sulfolane, H2O(2) H2SO4, NH4S2O8, H2O" ; O=C(O)C1CC(F)(F)C1 ; COc1cc(Cl)nnc1C1CC(F)(F)C1]

To a solution of 3-chloro-5-methoxy-pyridazine (15.00 g, 103.8 mmol) in sulfolane (30 mL), water (150 mL), and acetonitrile (30 mL) were added 3,3-difluorocyclobutanecarboxylic acid (28.24 g, 207.5 mmol) and silver nitrate (17.63 g, 103.8 mmol). The resulting reaction mixture was heated to 50° C. A solution of sulfuric acid (29.4 g, 300 mmol, 16.0 mL) in water (45 mL) was then added, followed by dropwise addition of a solution of ammonia sulfoxy hydrogen sulfate (39.00 g, 170.9 mmol, 37.14 mL) in water (45 mL) over 35 minutes. The reaction was warmed to 70° C. and stirred for 20 minutes, and was then cooled to room temperature and stirred for 24 hours. The reaction was then cooled to 0° C., neutralized to pH=7 by addition of aqueous sodium hydroxide solution (30% v/v, 15 mL), diluted with water (100 mL), and extracted with ethyl acetate (3×200 mL). The organic extracts were combined, washed with saturated aqueous sodium chloride solution (3×100 mL), dried over anhydrous sodium sulfate, filtered, and concentrated under reduced pressure. The resulting crude product was purified by flash chromatography on silica gel (0-100% ethyl acetate in petroleum ether) to provide the title compound: LCMS m/z 235.1 [M+H]⁺; ¹H NMR (400 MHz, CD₃OD) δ 7.35 (s, 1H), 4.00 (s, 3H), 3.76-3.68 (m, 1H), 3.07-2.90 (m, 4H).

### Step B. tert-butyl (6-(3,3-difluorocyclobutyl)-5-methoxypyridazin-3-yl)carbamate

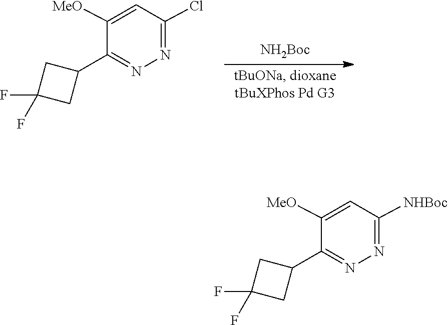 [SMILES: COc1cc(Cl)nnc1C1CC(F)(F)C1 ; "tBuONa, dioxanetBuXPhos Pd G3" ; COc1cc(NC(=O)OC(C)(C)C)nnc1C1CC(F)(F)C1 ; "NH2Boc"]

A mixture of 6-chloro-3-(3,3-difluorocyclobutyl)-4-methoxy-pyridazine (4.50 g, 19.2 mmol), tert-butyl carbamate (6.74 g, 57.5 mmol), sodium tert-butoxide solution (2 M in THF, 19.2 mL), and [2-(2-aminophenyl)phenyl]-methylsulfonyloxypalladium ditert-butyl-[2-(2,4,6-triisopropylphenyl)phenyl]phosphane (1.52 g, 1.92 mmol) in dioxane (100 mL) was purged with nitrogen, and was then heated at 100° C. for 16 hours under nitrogen atmosphere. The reaction was then cooled to the room temperature, diluted with water (200 mL), and extracted with ethyl acetate (3×200 mL). The organic extracts were combined, washed with saturated aqueous sodium chloride solution (3×100 mL), dried over sodium sulfate, filtered, and concentrated under reduced pressure. The resulting crude product was purified by flash chromatography on silica gel (0-100% ethyl acetate in petroleum ether) to provide the title compound: LCMS m/z 316.1 [M+H]⁺.

### Step C. 6-(3,3-difluorocyclobutyl)-5-methoxypyridazin-3-amine

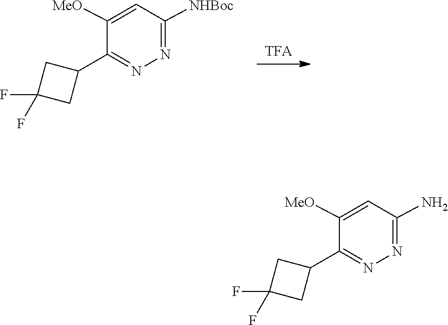 [SMILES: COc1cc(NC(=O)OC(C)(C)C)nnc1C1CC(F)(F)C1 ; COc1cc(N)nnc1C1CC(F)(F)C1 ; "TFA"]

A solution of tert-butyl N-[6-(3,3-difluorocyclobutyl)-5-methoxy-pyridazin-3-yl]carbamate (6.00 g, 19.0 mmol) in trifluoroacetic acid (20 mL) was stirred at 0° C. for 30 minutes under nitrogen atmosphere. The reaction was then concentrated under reduced pressure to provide the title compound: LCMS m/z 216.1 [M+H]⁺.

### Step D. 6-(3,3-difluorocyclobutyl)-7-methoxyimidazo[1,2-b]pyridazine

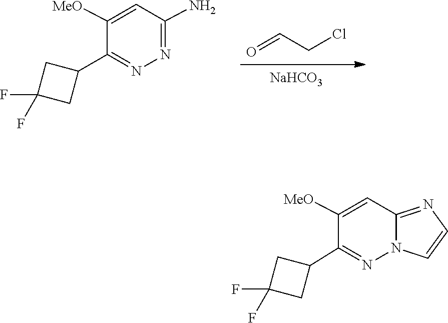

To a solution of 6-(3,3-difluorocyclobutyl)-5-methoxy-pyridazin-3-amine (4.00 g, 18.6 mmol) and 2-chloroacetaldehyde (18.24 g, 92.94 mmol, 14.95 mL, 40% purity) in ethanol (20 mL) was added sodium bicarbonate (3.12 g, 37.2 mmol). The resulting reaction mixture was stirred at 80° C. for 4 hours. The reaction was then cooled to room temperature, diluted with water (50 mL), and extracted with ethyl acetate (3×50 mL). The organic extracts were combined, washed with saturated aqueous sodium chloride solution (3×30 mL), dried over sodium sulfate, filtered, and concentrated under reduced pressure. The resulting crude product was purified by flash chromatography on silica gel (0-100% ethyl acetate in petroleum ether) to provide the title compound: LCMS m/z 240.1 [M+H]⁺; ¹H NMR (400 MHz, CD₃OD) δ 7.92-7.89 (m, 1H), 7.51 (d, J=1.4 Hz, 1H), 7.26 (s, 1H), 3.96 (s, 3H), 3.76-3.65 (m, 1H), 3.03-2.90 (m, 4H).

### Step E. 6-(3,3-difluorocyclobutyl)-3-iodo-7-methoxyimidazo[1,2-b]pyridazine

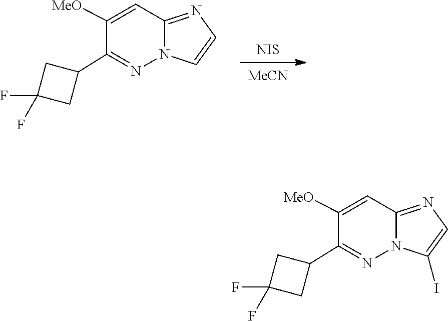

To a cooled 0° C. solution of 6-(3,3-difluorocyclobutyl)-7-methoxy-imidazo[1,2-b]pyridazine (1.00 g, 4.18 mmol) in acetonitrile (10 mL) was added 1-iodopyrrolidine-2,5-dione (1.03 g, 4.60 mmol). The resulting reaction was stirred for 1 hour while warming to room temperature. The reaction was then filtered and concentrated under reduced pressure to provide the title compound: LCMS m/z 365.8 [M+H]⁺; ¹H NMR (400 MHz, CD₃OD) δ 7.80 (s, 1H), 7.42 (s, 1H), 4.04 (s, 3H), 3.80-3.79 (m, 1H), 3.11-3.00 (m, 4H).

### Step F. 3-(2-chloropyrimidin-4-yl)-6-(3,3-difluorocyclobutyl)-7-methoxyimidazo[1,2-b]pyridazine

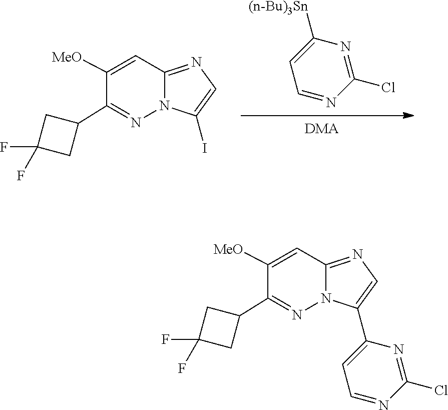

A mixture of 6-(3,3-difluorocyclobutyl)-3-iodo-7-methoxy-imidazo[1,2-b]pyridazine (0.700 g, 1.92 mmol), tributyl-(2-chloropyrimidin-4-yl)stannane (1.16 g, 2.88 mmol), and [1,1-bis(diphenylphosphino)ferrocene]dichloropalladium(II) (0.135 g, 0.192 mmol) in N,N-dimethylacetamide (10 mL) was purged with nitrogen, and was then heated at 100° C. for 16 hours under nitrogen atmosphere. The reaction was then cooled to room temperature, diluted with water (40 mL), and extracted with ethyl acetate (3×30 mL). The organic extracts were combined, washed with saturated aqueous sodium chloride solution (2×25 mL), dried over sodium sulfate, filtered, and concentrated under reduced pressure. The resulting crude product was purified by HPLC (Waters Xbridge BEH C18 column, 10 micron, 100×30 mm; 30-70% acetonitrile in water containing 0.1% sodium bicarbonate) to provide the title compound: LCMS m/z 352.1 [M+H]⁺.

### Step G. (3S,4S)-tert-butyl 3-((4-(6-(3,3-difluorocyclobutyl)-7-methoxyimidazo[1,2-b]pyridazin-3-yl)pyrimidin-2-yl)amino)-4-fluoropiperidine-1-carboxylate

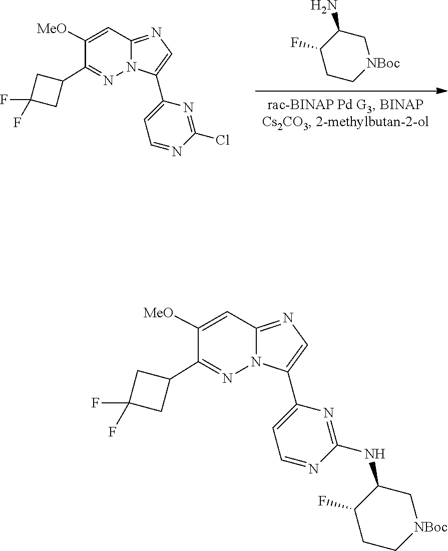

A mixture of 3-(2-chloropyrimidin-4-yl)-6-(3,3-difluorocyclobutyl)-7-methoxy-imidazo[1,2-b]pyridazine (0.035 mg, 0.100 mmol), tert-butyl (3S,4S)-3-amino-4-fluoropiperidine-1-carboxylate (0.026 g, 0.119 mmol), cesium carbonate (0.081 g, 0.249 mmol), [2-(2-aminophenyl)phenyl]-methylsulfonyloxypalladium[1-(2-diphenylphosphanyl-1-naphthyl)-2-naphthyl]-diphenylphosphane (0.010 g, 0.010 mmol), and (R)-(+)-2,2′-bis(diphenylphosphino)-1,1′-binaphthyl (0.009 g, 0.015 mmol) in 2-methylbutan-2-ol (3 mL) was purged with nitrogen, and was then heated at 80° C. for 2 hours under nitrogen atmosphere. The reaction was then cooled to room temperature, filtered, and concentrated under reduced pressure to provide the title compound: LCMS m/z 534.2 [M+H]⁺.

### Step H. 4-(6-(3,3-difluorocyclobutyl)-7-methoxyimidazo[1,2-b]pyridazin-3-yl)-N-((3S,4S)-4-fluoropiperidin-3-yl)pyrimidin-2-amine

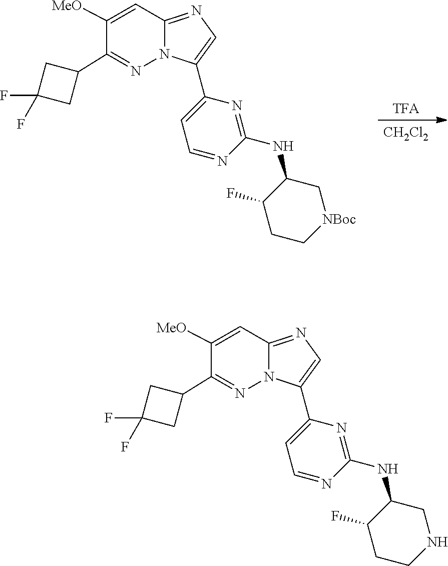 [SMILES: COc1cc2ncc(-c3ccnc(N[C@H]4CN(C(=O)OC(C)(C)C)CC[C@@H]4F)n3)n2nc1C1CC(F)(F)C1 ; "CH2Cl2" ; "TFA" ; COc1cc2ncc(-c3ccnc(N[C@H]4CNCC[C@@H]4F)n3)n2nc1C1CC(F)(F)C1]

To a solution of tert-butyl (3S,4S)-3-[[4-[6-(3,3-difluorocyclobutyl)-7-methoxy-imidazo[1,2-b]pyridazin-3-yl]pyrimidin-2-yl]amino]-4-fluoro-piperidine-1-carboxylate (0.050 g, 0.094 mmol) in dichloromethane (1 mL) was added trifluoroacetic acid (0.685 g, 6.01 mmol, 0.445 mL). The resulting reaction was stirred at room temperature for 1 hour, and was then concentrated under reduced pressure. The crude product thus obtained was purified by flash chromatography on silica gel (0-100% ethyl acetate in petroleum ether). The product thus obtained was further purified by HPLC (Phenomenex Luna C18 column, 5 micron, 150×30 mm, 10-40% acetonitrile in water containing 0.1% trifluoroacetic acid) to provide the title compound: LCMS m/z 434.1 [M+H]⁺; ¹H NMR (400 MHz, CD₃OD) δ 8.50 (d, J=5.4 Hz, 2H), 7.95 (d, J=5.4 Hz, 1H), 7.53 (s, 1H), 5.01-4.83 (m, 1H), 4.60-4.47 (m, 1H), 4.07 (s, 3H), 3.92-3.80 (m, 1H), 3.73-3.64 (m, 1H), 3.56-3.44 (m, 1H), 3.26-3.03 (m, 6H), 2.53-2.36 (m, 1H), 2.20-2.00 (m, 1H).

The compounds in Table 8 were all prepared using the synthetic procedures described in Example 9.

TABLE 8
Additional compounds prepared according to Example 9.

| Compound # | Structure | IUPAC Name | LCMS |
|---|---|---|---|
| 20a | 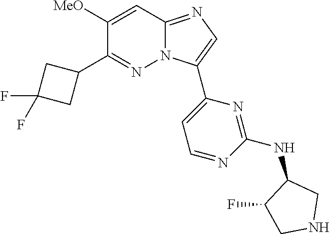 [SMILES: COc1cc2ncc(-c3ccnc(N[C@H]4CNC[C@@H]4F)n3)n2nc1C1CC(F)(F)C1] | 4-(6-(3,3- difluorocyclobutyl)-7- methoxyimidazo[1,2- b]pyridazin-3-yl)-N- ((3S,4S)-4- fluoropyrrolidin-3- yl)pyrimidin-2-amine | 420.1 |
| 21a | 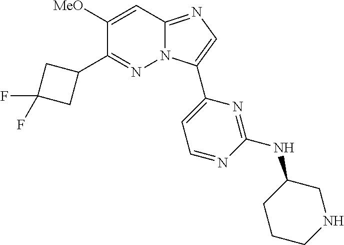 [SMILES: COc1cc2ncc(-c3ccnc(N[C@@H]4CCCNC4)n3)n2nc1C1CC(F)(F)C1] | (R)-4-(6-(3,3- difluorocyclobutyl)-7- methoxyimidazo[1,2- b]pyridazin-3-yl)-N- (piperidin-3-yl)pyrimidin- 2-amine | 416.2 |

### Example 10

### Exemplary Synthetic Procedure #8 (Compounds 22a-27a)

### Compounds 22a & 23a, Fast-Eluting Diastereomer of 1,1,1-trifluoro-2-(3-(2-((R)-piperidin-3-ylamino)pyrimidin-4-yl)imidazo[1,2-a]pyridin-6-yl)propan-2-ol & Slow-Eluting Diastereomer of 1,1,1-trifluoro-2-(3-(2-((R)-piperidin-3-ylamino)pyrimidin-4-yl)imidazo[1,2-a]pyridin-6-yl)propan-2-ol

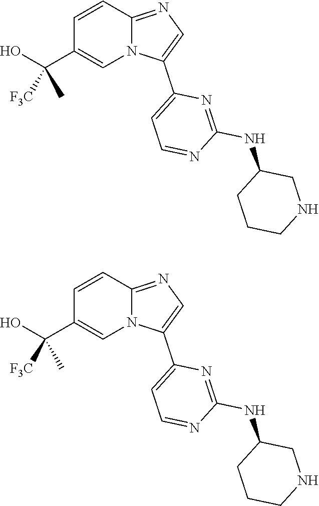 [SMILES: C[C@@](O)(c1ccc2ncc(-c3ccnc(N[C@@H]4CCCNC4)n3)n2c1)C(F)(F)F ; C[C@](O)(c1ccc2ncc(-c3ccnc(N[C@@H]4CCCNC4)n3)n2c1)C(F)(F)F]

### Step A. 6-(1-ethoxyvinyl)imidazo[1,2-a]pyridine

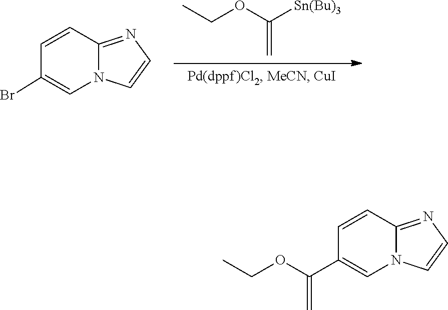

A 250 mL round-bottom flask equipped with a stirrer bar was charged with 6-bromoimidazo[1,2-a]pyridine (30.00 g, 152.3 mmol), acetonitrile (300 mL), tributyl(1-ethoxyvinyl)stannane (164.97 g, 456.78 mmol, 154.17 mL), [1,1-bis(diphenylphosphino)ferrocene]dichloropalladium(II) (11.14 g, 15.23 mmol), and cuprous iodide (4.35 g, 22.84 mmol). The resulting reaction was purged with nitrogen, and was then heated at 80° C. for 16 hours under nitrogen atmosphere. The reaction was then cooled room temperature, quenched by addition of aqueous potassium fluoride solution (20% w/w, 200 mL), and extracted with ethyl acetate (3×100 mL). The organic extracts were combined, washed with saturated aqueous sodium chloride solution (100 mL), dried over sodium sulfate, filtered, and concentrated under reduced pressure. The resulting crude product was purified by flash chromatography on silica gel (0-100% ethyl acetate in petroleum ether) to provide the title compound: LCMS m/z 188.1 [M+H]⁺; ¹H NMR (400 MHz, CD₃OD) δ 8.93 (br s, 1H), 8.62-8.00 (m, 1H), 7.98-7.26 (m, 3H), 4.79 (d, J=3.1 Hz, 1H), 4.40 (d, J=3.0 Hz, 1H), 3.99 (q, J=7.0 Hz, 2H), 1.46 (t, J=7.0 Hz, 3H).

### Step B. 1-(imidazo[1,2-a]pyridin-6-yl)ethanone

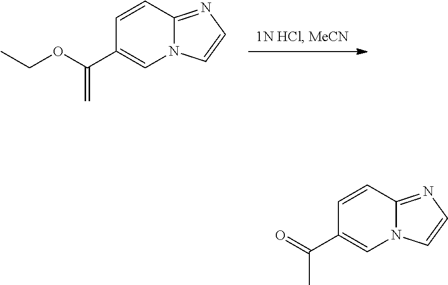

A 2 L round bottom flask equipped with a stirrer bar was charged with 6-(1-ethoxyvinyl)imidazo[1,2-a]pyridine (21.30 g, 113.2 mmol) and acetonitrile (200 mL). Hydrochloric acid (1 M, 113.2 mL) was then added, and the resulting reaction was stirred at room temperature for 1 hour. The reaction was then concentrated under reduced pressure to provide the title compound: LCMS m/z 161.0 [M+H]⁺.

### Step C. 1,1,1-trifluoro-2-(imidazo[1,2-a]pyridin-6-yl)propan-2-ol

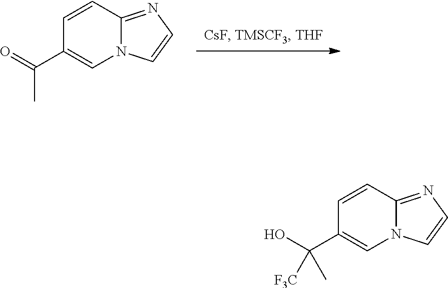

To a cooled 0° C. solution of 1-(imidazo[1,2-a]pyridin-6-yl)ethanone (7.25 g, 45.26 mmol) and tetrahydrofuran (120 mL) were added cesium fluoride (27.50 g, 181.1 mmol) and trimethyl(trifluoromethyl)silane (19.31 g, 135.8 mmol), in that order. The resulting reaction mixture was stirred for 10 hours while warming to room temperature. The reaction was then concentrated under reduced pressure to provide the title compound: LCMS m/z 231.0 [M+H]⁺.

### Step D. 1,1,1-trifluoro-2-(3-iodoimidazo[1,2-a]pyridin-6-yl)propan-2-ol

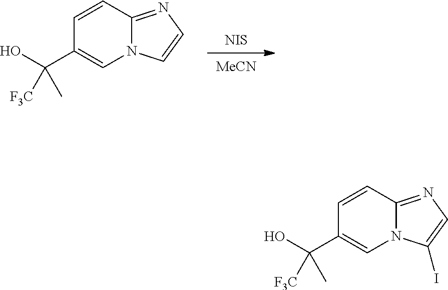

To a cooled 0° C. solution of 1,1,1-trifluoro-2-imidazo[1,2-a]pyridin-6-yl-propan-2-ol (1.00 g, 4.34 mmol) in acetonitrile (10 mL) was added 1-iodopyrrolidine-2,5-dione (0.977 g, 4.34 mmol). The resulting reaction was stirred for 2 hours while warming to room temperature. The reaction was then filtered and concentrated under reduced pressure to provide the title compound: LCMS m/z 356.8 [M+H]⁺.

### Step E. 2-(3-(2-chloropyrimidin-4-yl)imidazo[1,2-a]pyridin-6-yl)-1,1,1-trifluoropropan-2-ol

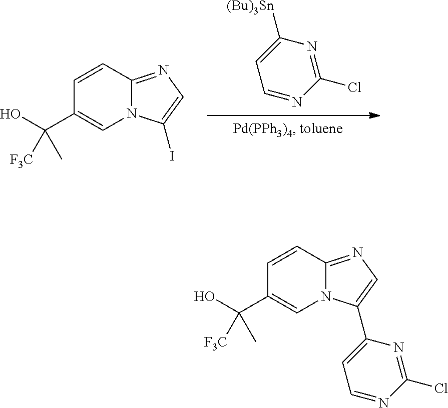

A mixture of 1,1,1-trifluoro-2-(3-iodoimidazo[1,2-a]pyridin-6-yl)propan-2-ol (0.950 g, 2.67 mmol), 2-chloro-4-(tributylstannyl)pyrimidine (1.62 g, 4.00 mmol), and tetrakis(triphenylphosphine)palladium(0) (0.308 g, 0.267 mmol) in toluene (15 mL) was purged with nitrogen, and was then heated at 100° C. for 16 hours under nitrogen atmosphere. The reaction was then cooled to the room temperature, filtered, and concentrated under reduced pressure. The resulting crude product was purified by HPLC (Phenomenex Luna C18 column, 5 micron, 100×30 mm; 25-50% acetonitrile in water containing 0.04% ammonium bicarbonate) to provide the title compound: LCMS m/z 343.0 [M+H]⁺

### Step F. (3R)-tert-butyl 3-((4-(6-(1,1,1-trifluoro-2-hydroxypropan-2-yl)imidazo[1,2-a]pyridin-3-yl)pyrimidin-2-yl)amino)piperidine-1-carboxylate

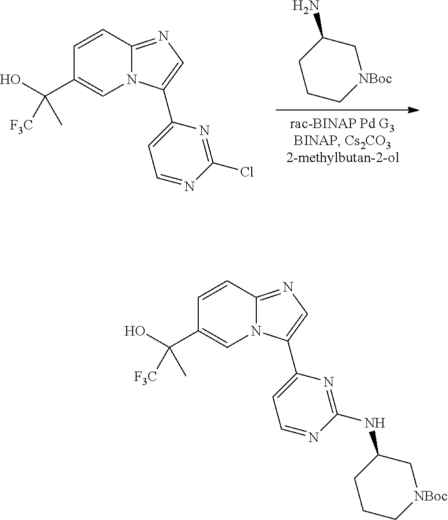

A mixture of 2-(3-(2-chloropyrimidin-4-yl)imidazo[1,2-a]pyridin-6-yl)-1,1,1-trifluoropropan-2-ol (0.070 mg, 0.204 mmol), (R)-tert-butyl 3-aminopiperidine-1-carboxylate (0.045 g, 0.225 mmol), methanesulfonato[2,2-bis(diphenylphosphino)-1,1-binaphthyl](2-amino-1,1-biphenyl-2-yl)palladium(II) (0.020 g, 0.020 mmol), (R)-(+)-2,2′-bis(diphenylphosphino)-1,1′-binaphthyl (0.013 g, 0.020 mmol), and cesium carbonate (0.200 g, 0.613 mmol) in 2-methylbutan-2-ol (4 mL) was purged with nitrogen, and was then heated at 80° C. for 5 hours under nitrogen atmosphere. The reaction was then cooled to the room temperature, diluted with water (5 mL), and extracted with ethyl acetate (3×5 mL). The organic extracts were combined, washed with saturated aqueous sodium chloride solution (5 mL), dried over sodium sulfate, filtered, and concentrated under reduced pressure to provide the title compound: LCMS m/z 507.1 [M+H]⁺.

### Step G. Fast-Eluting Diastereomer of 1,1,1-trifluoro-2-(3-(2-((R)-piperidin-3-ylamino)pyrimidin-4-yl)imidazo[1,2-a]pyridin-6-yl)propan-2-ol & Slow-Eluting Diastereomer of 1,1,1-trifluoro-2-(3-(2-((R)-piperidin-3-ylamino)pyrimidin-4-yl)imidazo[1,2-a]pyridin-6-yl)propan-2-ol

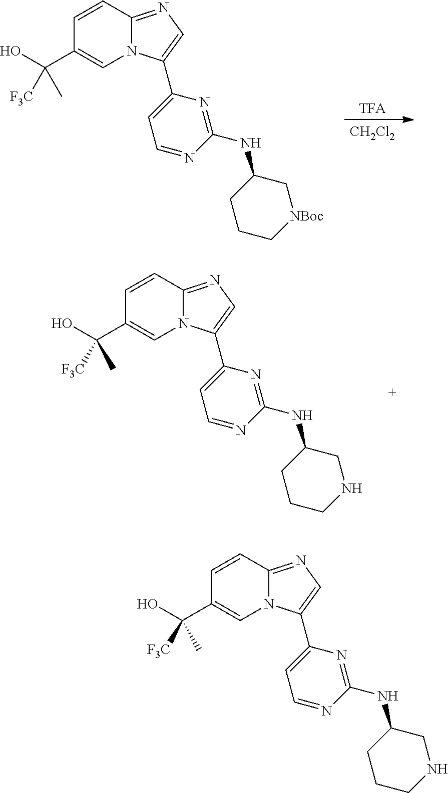 [SMILES: CC(C)(C)OC(=O)N1CCC[C@@H](Nc2nccc(-c3cnc4ccc(C(C)(O)C(F)(F)F)cn34)n2)C1 ; "CH2Cl2" ; C[C@@](O)(c1ccc2ncc(-c3ccnc(N[C@@H]4CCCNC4)n3)n2c1)C(F)(F)F ; "TFA" ; C[C@](O)(c1ccc2ncc(-c3ccnc(N[C@@H]4CCCNC4)n3)n2c1)C(F)(F)F]

To a solution of (3R)-tert-butyl 3-((4-(6-(1,1,1-trifluoro-2-hydroxypropan-2-yl)imidazo[1,2-a]pyridin-3-yl)pyrimidin-2-yl)amino)piperidine-1-carboxylate (0.200 g, 0.395 mmol) in dichloromethane (3 mL) was added trifluoroacetic acid (0.770 g, 6.75 mmol, 0.50 mL). The resulting reaction was stirred at room temperature for 2 hours, and was then concentrated under reduced pressure. The crude product thus obtained was purified by HPLC (Phenomenex Luna C18 column, 5 micron, 80×30 mm; 1-30% acetonitrile in water containing 0.04% trifluoroacetic acid) to provide the title compounds: Fast-eluting diastereomer of 1,1,1-trifluoro-2-(3-(2-((R)-piperidin-3-ylamino)pyrimidin-4-yl)imidazo[1,2-a]pyridin-6-yl)propan-2-ol: LCMS m/z 407.1 [M+H]⁺; ¹H NMR (400 MHz, CD₃OD) δ 10.52 (s, 1H), 8.79-8.75 (s, 1H), 8.41 (d, J=5.6 Hz, 1H), 8.01-7.88 (m, 2H), 7.36 (br d, J=5.4 Hz, 1H), 4.54-4.40 (m, 1H), 3.60-3.56 (m, 1H), 3.32-3.28 (m, 2H), 3.24-3.14 (m, 1H), 2.26-2.15 (m, 2H), 2.00-1.91 (m, 5H). Slow-eluting diastereomer of 1,1,1-trifluoro-2-(3-(2-((R)-piperidin-3-ylamino)pyrimidin-4-yl)imidazo[1,2-a]pyridin-6-yl)propan-2-ol: LCMS m/z 407.0 [M+H]⁺; ¹H NMR (400 MHz, CD₃OD) δ 10.46 (s, 1H), 8.80 (s, 1H), 8.42 (d, J=5.6 Hz, 1H), 8.05 (d, J=9.5 Hz, 1H), 7.96 (d, J=9.1 Hz, 1H), 7.35 (d, J=5.5 Hz, 1H), 4.49 (br s, 1H), 3.54 (br d, J=11.4 Hz, 1H), 3.32 (m, 2H), 3.22-3.12 (m, 1H), 2.26-2.11 (m, 2H), 1.93 (m, 5H).

The compounds in Table 9 were all prepared using the synthetic procedures described in Example 10.

TABLE 9
Additional compounds prepared according to Example 10.

| Compound # | Structure | IUPAC Name | LCMS |
|---|---|---|---|
| 24a | 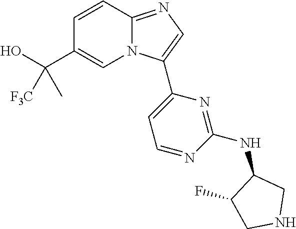 [SMILES: CC(O)(c1ccc2ncc(-c3ccnc(N[C@H]4CNC[C@@H]4F)n3)n2c1)C(F)(F)F] | fast-eluting diastereomer- 1,1,1-trifluoro-2-(3-(2- (((3S,4S)-4- fluoropyrrolidin-3- yl)amino)pyrimidin-4- yl)imidazo[1,2-a]pyridin- 6-yl)propan-2-ol | 411.0 |
| 25a | 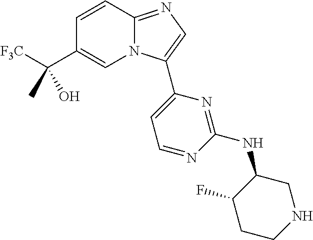 [SMILES: C[C@](O)(c1ccc2ncc(-c3ccnc(N[C@H]4CNCC[C@@H]4F)n3)n2c1)C(F)(F)F] | fast-eluting diastereomer- 1,1,1-trifluoro-2-(3-(2- (((3S,4S)-4- fluoropiperidin-3- yl)amino)pyrimidin-4- yl)imidazo[1,2-a]pyridin- 6-yl)propan-2-ol | 425.0 |
| 26a | 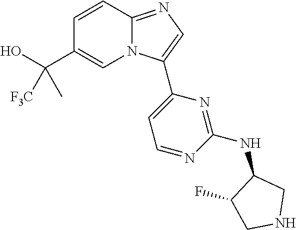 [SMILES: CC(O)(c1ccc2ncc(-c3ccnc(N[C@H]4CNC[C@@H]4F)n3)n2c1)C(F)(F)F] | slow-eluting diastereomer-(S)-1,1,1- trifluoro-2-(3-(2- (((3S,4S)-4- fluoropyrrolidin-3- yl)amino)pyrimidin-4- yl)imidazo[1,2-a]pyridin- 6-yl)propan-2-ol | 411.0 |
| 27a | 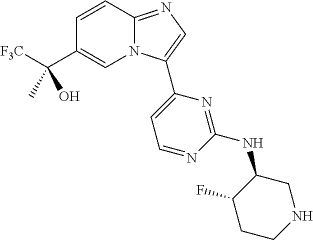 [SMILES: C[C@@](O)(c1ccc2ncc(-c3ccnc(N[C@H]4CNCC[C@@H]4F)n3)n2c1)C(F)(F)F] | slow-eluting diastereomer-1,1,1- trifluoro-2-(3-(2- (((3S,4S)-4- fluoropiperidin-3- yl)amino)pyrimidin-4- yl)imidazo[1,2-a]pyridin- 6-yl)propan-2-ol | 425.0 |

### Example 11

### Exemplary Synthetic Procedure #9 (Compounds 28a-33a,)

### Compound 28a & 29a, Fast-Eluting Diastereomer of 1,1,1-trifluoro-2-(3-(6-((R)-piperidin-3-ylamino)pyrazin-2-yl)imidazo[1,2-a]pyridin-6-yl)propan-2-ol & Slow-Eluting Diastereomer of 1,1,1-trifluoro-2-(3-(6-((R)-piperidin-3-ylamino)pyrazin-2-yl)imidazo[1,2-a]pyridin-6-yl)propan-2-ol

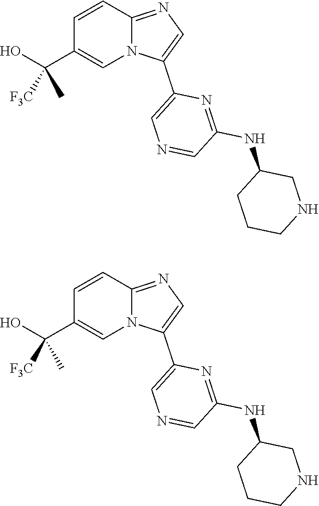 [SMILES: C[C@@](O)(c1ccc2ncc(-c3cncc(N[C@@H]4CCCNC4)n3)n2c1)C(F)(F)F ; C[C@](O)(c1ccc2ncc(-c3cncc(N[C@@H]4CCCNC4)n3)n2c1)C(F)(F)F]

### Step A. 2-(3-(6-bromopyrazin-2-yl)imidazo[1,2-a]pyridin-6-yl)-1,1,1-trifluoropropan-2-ol

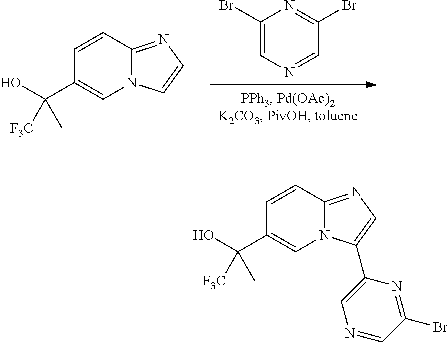

A mixture of 1,1,1-trifluoro-2-(imidazo[1,2-a]pyridin-6-yl)propan-2-ol (1.00 g, 4.34 mmol), 2,6-dibromopyrazine (2.07 g, 8.69 mmol), triphenylphosphine (0.171 g, 0.652 mmol), palladium(II)acetate (0.098 g, 0.434 mmol), potassium carbonate (3.00 g, 21.7 mmol), and 2,2-dimethylpropanoic acid (0.133 g, 1.30 mmol, 0.150 mL) in toluene (30 mL) was purged with nitrogen, and was then heated at 100° C. for 16 hours under nitrogen atmosphere. The reaction was cooled to room temperature and concentrated under reduced pressure. The resulting crude product was purified by flash chromatography on silica gel (0-100% ethyl acetate in petroleum ether) to provide the title compound: LCMS m/z 387.1 [M+H]⁺.

### Step B. (3R)-tert-butyl 3-((6-(6-(1,1,1-trifluoro-2-hydroxypropan-2-yl)imidazo[1,2-a]pyridin-3-yl)pyrazin-2-yl)amino)piperidine-1-carboxylate

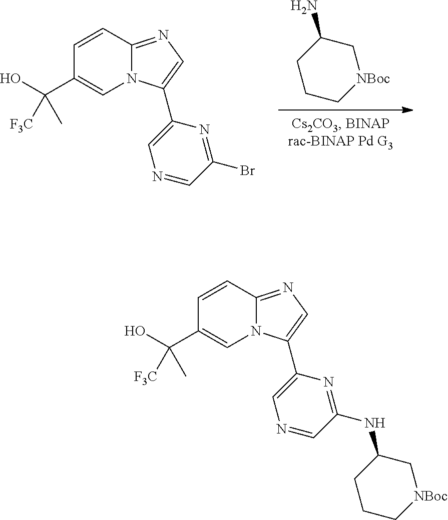

A mixture of 2-(3-(6-bromopyrazin-2-yl)imidazo[1,2-a]pyridin-6-yl)-1,1,1-trifluoropropan-2-ol (0.180 g, 0.465 mmol), (R)-tert-butyl 3-aminopiperidine-1-carboxylate (0.074 g, 0.372 mmol), methanesulfonato[2,2-bis(diphenylphosphino)-1,1-binaphthyl](2-amino-1,1-biphenyl-2-yl)palladium(II) (0.046 g, 0.046 mmol), (R)-(+)-2,2′-bis(diphenylphosphino)-1,1′-binaphthyl (0.029 g, 0.046 mmol), and cesium carbonate (0.0379 g, 1.16 mmol) in tetrahydrofuran (5 mL) was purged with nitrogen, and was then heated at 80° C. for 5 hours under nitrogen atmosphere. The reaction was cooled to room temperature, filtered, and concentrated under reduced pressure. The resulting crude product was purified by prep-TLC (silica gel, 10% ethyl acetate in petroleum ether) to provide the title compound: LCMS m/z 506.5 [M+H]⁺.

### Step C. Fast-Eluting Diastereomer of 1,1,1-trifluoro-2-(3-(6-((R)-piperidin-3-ylamino)pyrazin-2-yl)imidazo[1,2-a]pyridin-6-yl)propan-2-ol & Slow-Eluting Diastereomer of 1,1,1-trifluoro-2-(3-(6-((R)-piperidin-3-ylamino)pyrazin-2-yl)imidazo[1,2-a]pyridin-6-yl)propan-2-ol

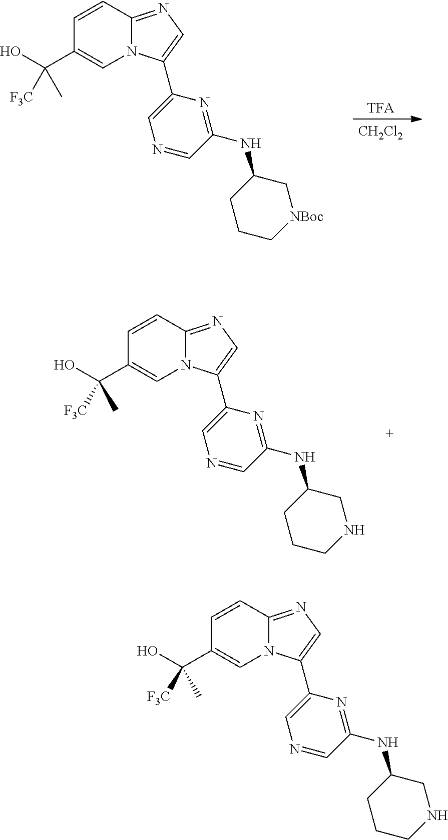 [SMILES: CC(C)(C)OC(=O)N1CCC[C@@H](Nc2cncc(-c3cnc4ccc(C(C)(O)C(F)(F)F)cn34)n2)C1 ; "CH2Cl2" ; "TFA" ; C[C@@](O)(c1ccc2ncc(-c3cncc(N[C@@H]4CCCNC4)n3)n2c1)C(F)(F)F ; C[C@](O)(c1ccc2ncc(-c3cncc(N[C@@H]4CCCNC4)n3)n2c1)C(F)(F)F]

To a solution of (3R)-tert-butyl 3-((6-(6-(1,1,1-trifluoro-2-hydroxypropan-2-yl)imidazo[1,2-a]pyridin-3-yl)pyrazin-2-yl)amino)piperidine-1-carboxylate (0.094 g, 0.186 mmol) in dichloromethane (3 mL) was added trifluoroacetic acid (0.770 g, 6.75 mmol, 0.50 mL). The resulting reaction was stirred at room temperature for 2 hours, and was then concentrated under reduced pressure. The crude product thus obtained was purified by HPLC (Phenomenex Luna C18 column, 5 micron, 100×40 mm; 15-60% acetonitrile in water containing 0.04% trifluoroacetic acid) to provide the title compound: Fast-eluting diastereomer: LCMS m/z 407.1 [M+H]⁺; ¹H NMR (400 MHz, CD₃OD) δ 10.30 (s, 1H), 8.73 (s, 1H), 8.35 (s, 1H), 8.12 (d, J=9.5 Hz, 1H), 8.03 (s, 1H), 7.97 (d, J=10.0 Hz, 1H), 4.35 (br s, 1H), 3.36-3.29 (m, 2H), 3.14 (br s, 2H), 2.15-2.06 (m, 2H), 1.90-1.82 (m, 2H), 1.82 (s, 3H). Slow-eluting diastereomer: LCMS m/z 407.1 [M+H]⁺; ¹H NMR (400 MHz, CD₃OD) δ 10.24 (s, 1H), 8.69 (s, 1H), 8.42 (s, 1H), 8.11 (m, 1H), 8.09 (s, 1H), 8.01 (d, J=9.5 Hz, 1H), 4.46 (br s, 1H), 3.48-3.39 (m, 2H), 3.25 (br s, 2H), 2.20-2.18 (m, 2H), 2.02-1.94 (m, 2H), 1.88 (s, 3H)

The compounds in Table 10 were all prepared using the synthetic procedures described in Example 11.

TABLE 10
Additional compounds prepared according to Example 11.

| Compound # | Structure | IUPAC Name | LCMS |
|---|---|---|---|
| 30a | 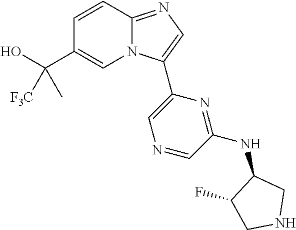 [SMILES: CC(O)(c1ccc2ncc(-c3cncc(N[C@H]4CNC[C@@H]4F)n3)n2c1)C(F)(F)F] | fast-eluting diastereomer- 1,1,1-trifluoro-2-(3-(6- (((3S,4S)-4- fluoropyrrolidin-3- yl)amino)pyrazin-2- yl)imidazo[1,2-a]pyridin- 6-yl)propan-2-ol | 410.1 |
| 31a | 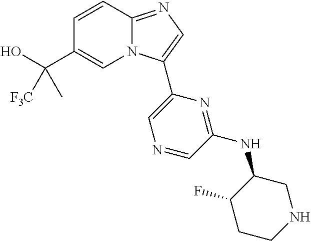 [SMILES: CC(O)(c1ccc2ncc(-c3cncc(N[C@H]4CNCC[C@@H]4F)n3)n2c1)C(F)(F)F] | fast-eluting diastereomer- 1,1,1-trifluoro-2-(3-(6- (((3S,4S)-4- fluoropiperidin-3- yl)amino)pyrazin-2- yl)imidazo[1,2-a]pyridin- 6-yl)propan-2-ol | 424.1 |
| 32a | 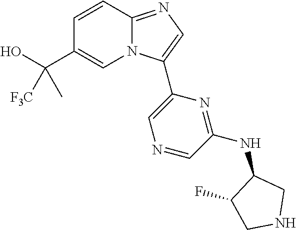 [SMILES: CC(O)(c1ccc2ncc(-c3cncc(N[C@H]4CNC[C@@H]4F)n3)n2c1)C(F)(F)F] | slow-eluting diastereomer-1,1,1- trifluoro-2-(3-(6- (((3S,4S)-4- fluoropyrrolidin-3- yl)amino)pyrazin-2- yl)imidazo[1,2-a]pyridin- 6-yl)propan-2-ol | 410.1 |
| 33a | 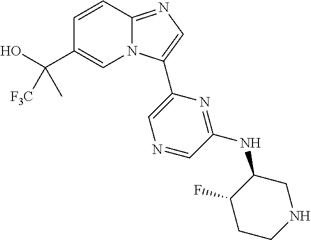 [SMILES: CC(O)(c1ccc2ncc(-c3cncc(N[C@H]4CNCC[C@@H]4F)n3)n2c1)C(F)(F)F] | slow-eluting diastereomer-1,1,1- trifluoro-2-(3-(6- (((3S,4S)-4- fluoropiperidin-3- yl)amino)pyrazin-2- yl)imidazo[1,2-a]pyridin- 6-yl)propan-2-ol | 424.1 |

### Example 12

### Exemplary Synthetic Procedure #10 (Compounds 34a-39a) Compounds 34a & 35a, Fast-Eluting Diastereomer of 1,1,1-trifluoro-2-(3-(5-fluoro-2-(((3S,4S)-4-fluoropiperidin-3-yl)amino)pyrimidin-4-yl)imidazo[1,2-a]pyridin-6-yl)propan-2-ol & Slow-Eluting Diastereomer of 1,1,1-trifluoro-2-(3-(5-fluoro-2-(((3S,4S)-4-fluoropiperidin-3-yl)amino)pyrimidin-4-yl)imidazo[1,2-a]pyridin-6-yl)propan-2-ol

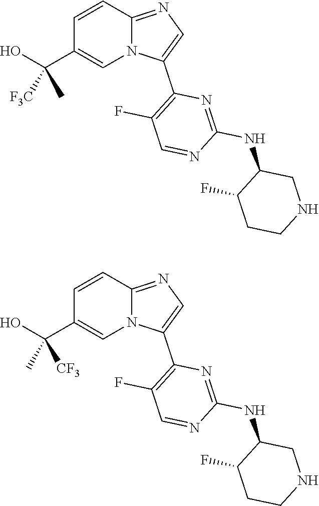 [SMILES: C[C@@](O)(c1ccc2ncc(-c3nc(N[C@H]4CNCC[C@@H]4F)ncc3F)n2c1)C(F)(F)F ; C[C@](O)(c1ccc2ncc(-c3nc(N[C@H]4CNCC[C@@H]4F)ncc3F)n2c1)C(F)(F)F]

### Step A. 2-(6-chloropyridin-3-yl)-1,1,1-trifluoropropan-2-ol

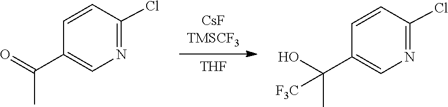

To a cooled 0° C. solution of 1-(6-chloro-3-pyridyl)ethanone (4.50 g, 28.9 mmol) and cesium fluoride (8.79 g, 57.9 mmol, 2.13 mL) in tetrahydrofuran (45 mL) was added dropwise trimethyl(trifluoromethyl)silane (20.56 g, 144.6 mmol). The resulting reaction was stirred for 16 hours while warming to room temperature. The reaction was then diluted with water (50 mL) and extracted with ethyl acetate (3×50 mL). The organic extracts were combined, washed with saturated aqueous sodium chloride solution (30 mL), dried over anhydrous sodium sulfate, filtered, and concentrated under reduced pressure. The resulting crude product was purified by flash chromatography on silica gel (0-80% ethyl acetate in petroleum ether) to provide the title compound: LCMS m/z 225.90 [M+H]⁻; ¹HNMR (400 MHz, CD₃OD) δ 8.58 (d, J=2.5 Hz, 1H), 8.04 (dd, J=2.3, 8.4 Hz, 1H), 7.49 (d, J=8.4 Hz, 1H), 1.76 (s, 3H).

### Step B. tert-butyl (5-(1,1,1-trifluoro-2-hydroxypropan-2-yl)pyridin-2-yl)carbamate

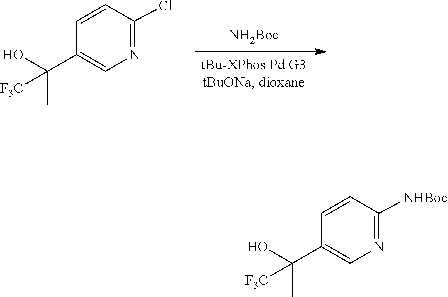 [SMILES: CC(O)(c1ccc(Cl)nc1)C(F)(F)F ; "tBu-XPhos Pd G3tBuONa, dioxane" ; CC(C)(C)OC(=O)Nc1ccc(C(C)(O)C(F)(F)F)cn1 ; "NH2Boc"]

A mixture of 2-(6-chloro-3-pyridyl)-1,1,1-trifluoro-propan-2-ol (3.50 g, 15.5 mmol), tert-butyl carbamate (5.45 g, 46.5 mmol), [2-(2-aminophenyl)phenyl]-methylsulfonyloxy-palladiumditert-butyl-[2-(2,4,6-triisopropylphenyl)phenyl]phosphane (1.23 g, 1.55 mmol), and sodium tert-butoxide solution (2 M in THF, 23.3 mL) in dioxane (50 mL) was purged with nitrogen, and was then heated at 100° C. for 16 hours under nitrogen atmosphere. The reaction was cooled to room temperature, diluted with water (50 mL), and extracted with ethyl acetate (3×100 mL). The organic extracts were combined, washed with saturated aqueous sodium chloride solution (100 mL), dried over anhydrous sodium sulfate, filtered, and concentrated under reduced pressure. The resulting crude product was purified by flash chromatography on silica gel (0-10% ethyl acetate in petroleum ether) to provide the title compound: LCMS m/z 307.10 [M+H]⁺.

### Step C. 2-(6-aminopyridin-3-yl)-1,1,1-trifluoropropan-2-ol

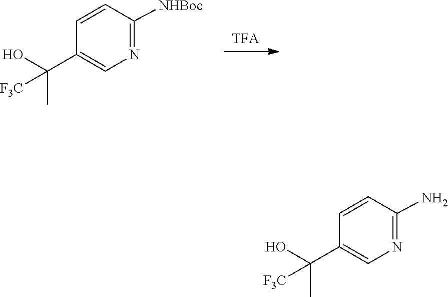 [SMILES: CC(C)(C)OC(=O)Nc1ccc(C(C)(O)C(F)(F)F)cn1 ; CC(O)(c1ccc(N)nc1)C(F)(F)F ; "TFA"]

A solution of tert-butyl N-[5-(2,2,2-trifluoro-1-hydroxy-1-methyl-ethyl)-2-pyridyl]carbamate (2.00 g, 6.53 mmol) in trifluoroacetic acid (13 mL) was stirred at room temperature for 1 hour. The reaction was then concentrated under reduced pressure to provide the title compound: LCMS m/z 206.95 [M+H]⁺.

### Step D. 2-(3-(2-bromo-5-fluoropyrimidin-4-yl)imidazo[1,2-a]pyridin-6-yl)-1,1,1-trifluoropropan-2-ol

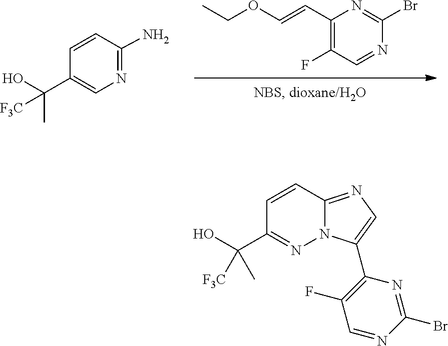

To a cooled 0° C. solution of 2-bromo-4-[(E)-2-ethoxyvinyl]-5-fluoro-pyrimidine (1.85 g, 7.50 mmol) in dioxane (20 mL) and water (8.5 mL) was added 1-bromopyrrolidine-2,5-dione (1.33 g, 7.50 mmol). The resulting reaction was removed from the cold bath and stirred for 30 minutes while warming to room temperature. 2-(6-Amino-3-pyridyl)-1,1,1-trifluoro-propan-2-ol (2.00 g, 6.25 mmol) was then added, and the reaction was heated to 80° C. for 2 hours. The reaction was then cooled to room temperature, diluted with water (50 mL), and extracted with ethyl acetate (3×50 mL). The organic extracts were combined, washed with saturated aqueous sodium chloride solution (50 mL), dried over anhydrous sodium sulfate, filtered, and concentrated under reduced pressure. The resulting crude product was purified by flash chromatography on silica gel (0-50% ethyl acetate in petroleum ether) to provide the title compound: LCMS m/z 406.95 [M+H]⁻; ¹HNMR (400 MHz, CD₃OD) δ 10.36 (s, 1H), 8.62 (d, J=3.6 Hz, 1H), 8.57 (d, J=3.8 Hz, 1H), 7.95 (d, J=9.8 Hz, 1H), 7.84 (d, J=9.6 Hz, 1H), 1.88 (s, 3H).

### Step E. (3S,4S)-tert-butyl 4-fluoro-3-((5-fluoro-4-(6-((R)-1,1,1-trifluoro-2-hydroxypropan-2-yl)imidazo[1,2-a]pyridin-3-yl)pyrimidin-2-yl)amino)piperidine-1-carboxylate

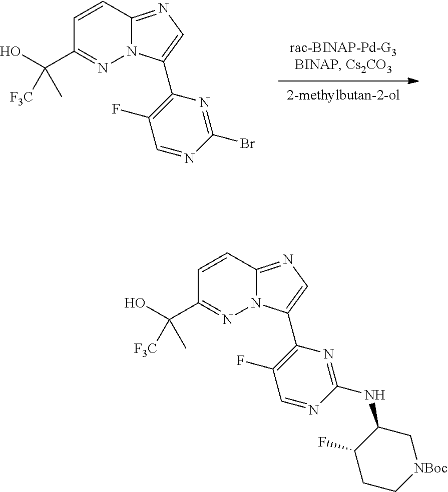 [SMILES: CC(O)(c1ccc2ncc(-c3nc(Br)ncc3F)n2n1)C(F)(F)F ; "2-methylbutan-2-ol" ; "rac-BINAP-Pd-G3BINAP, Cs2CO3" ; CC(C)(C)OC(=O)N1CC[C@H](F)[C@@H](Nc2ncc(F)c(-c3cnc4ccc(C(C)(O)C(F)(F)F)nn34)n2)C1]

A mixture of 2-[3-(2-bromo-5-fluoro-pyrimidin-4-yl)imidazo[1,2-a]pyridin-6-yl]-1,1,1-trifluoro-propan-2-ol (0.100 g, 0.247 mmol), tert-butyl (3S,4S)-3-amino-4-fluoro-piperidine-1-carboxylate (0.081 g, 0.370 mmol), methanesulfonato[2,2-bis(diphenylphosphino)-1,1-binaphthyl](2-amino-1,1-biphenyl-2-yl)palladium(II) (0.024 g, 0.025 mmol), (R)-(+)-2,2′-bis(diphenylphosphino)-1,1′-binaphthyl (0.023 g, 0.037 mmol), and cesium carbonate (0.201 g, 0.617 mmol) in 2-methylbutan-2-ol (7 mL) was purged with nitrogen, and was then heated at 80° C. for 4 hours under nitrogen atmosphere. The reaction was cooled to room temperature, filtered, and concentrated under reduced pressure to provide the title compound: LCMS m/z 543.10 [M+H]⁻.

### Step F. Fast-Eluting diastereomer-1,1,1-trifluoro-2-(3-(5-fluoro-2-(((3S,4S)-4-fluoropiperidin-3-yl)amino)pyrimidin-4-yl)imidazo[1,2-a]pyridin-6-yl)propan-2-ol & Slow-Eluting diastereomer-1,1,1-trifluoro-2-(3-(5-fluoro-2-(((3S,4S)-4-fluoropiperidin-3-yl)amino)pyrimidin-4-yl)imidazo[1,2-a]pyridin-6-yl)propan-2-ol

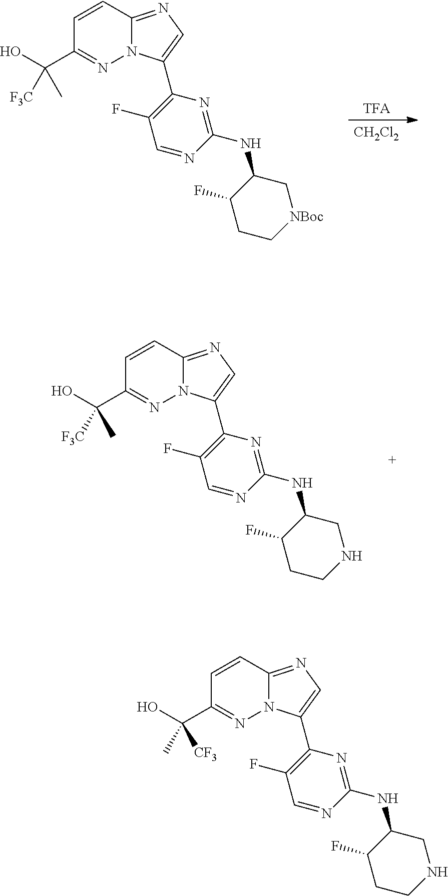 [SMILES: CC(C)(C)OC(=O)N1CC[C@H](F)[C@@H](Nc2ncc(F)c(-c3cnc4ccc(C(C)(O)C(F)(F)F)nn34)n2)C1 ; "CH2Cl2" ; "TFA" ; C[C@@](O)(c1ccc2ncc(-c3nc(N[C@H]4CNCC[C@@H]4F)ncc3F)n2n1)C(F)(F)F ; C[C@](O)(c1ccc2ncc(-c3nc(N[C@H]4CNCC[C@@H]4F)ncc3F)n2n1)C(F)(F)F]

To a solution of tert-butyl (3S,4S)-4-fluoro-3-[[5-fluoro-4-[6-[(1R)-2,2,2-trifluoro-1-hydroxy-1-methyl-ethyl]imidazo[1,2-a]pyridin-3-yl]pyrimidin-2-yl]amino]piperidine-1-carboxylate (0.056 g, 0.103 mmol) in dichloromethane (4 mL) was added trifluoroacetic acid (1.54 g, 13.5 mmol, 1 mL). The resulting reaction was stirred at room temperature for 1 hour, and was then filtered and concentrated under reduced pressure. The crude product thus obtained was purified by HPLC (Phenomenex Luna C18 column, 3 micron, 80×30 mm; 1-20% acetonitrile in water containing 0.1% hydrochloric acid) to provide the title compound: Fast-eluting diastereomer: LCMS m/z 443.10 [M+H]⁺; ¹HNMR (400 MHz, CD₃OD) δ 10.52 (s, 1H), 8.84 (d, J=1.9 Hz, 1H), 8.60 (d, J=2.9 Hz, 1H), 8.33 (d, J=9.4 Hz, 1H), 8.15 (d, J=9.5 Hz, 1H), 5.05-5.01 (m, 1H), 4.59 (mn, 1H), 3.66 (br d, J=13.0 Hz, 1H), 3.50-3.35 (mn, 2H), 3.30-3.17 (m, 1H), 3.29-3.20 (m, 1H), 2.55-2.44 (m, 1H), 2.25-2.12 (m, 1H), 1.95 (s, 3H). Slow-eluting diastereomer: LCMS m/z 443.10 [M+H]⁺; ¹HNMR (400 z, CD3D) 6 10.50 (s, 1H), 8.83 (d, J=2.3 Hz, 1H), 8.59 (d, J=3.0 Hz, 1H), 8.33 (d, J=9.5 Hz, 1H), 8.13 (d, J=9.5 Hz, 1H), 5.01-4.95 (m, 1H), 4.64-4.56 (m, 1H), 3.70-3.58 (m, 1H), 3.51-3.41 (m, 1H), 3.30-3.20 (m, 2H), 2.57-2.41 (mn, 1H), 2.26-2.07 (m, 1H), 1.95 (s, 3H).

The compounds in Table 11 were all prepared using the synthetic procedures described in Example 12.

TABLE 11
Additional compounds prepared according to Example 12.

| Compound # | Structure | IUPAC Name | LCMS |
|---|---|---|---|
| 36a | 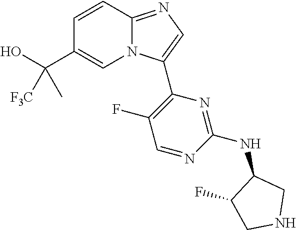 [SMILES: CC(O)(c1ccc2ncc(-c3nc(N[C@H]4CNC[C@@H]4F)ncc3F)n2c1)C(F)(F)F] | Fast-eluting diastereomer- 1,1,1-trifluoro-2-(3-(5- fluoro-2-(((3S,4S)-4- fluoropyrrolidin-3- yl)amino)pyrimidin-4- yl)imidazo[1,2-a]pyridin- 6-yl)propan-2-ol | 429.14 |
| 37a | 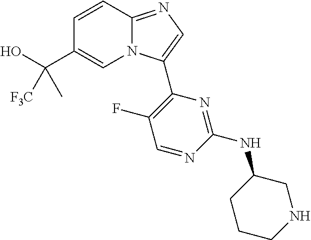 [SMILES: CC(O)(c1ccc2ncc(-c3nc(N[C@@H]4CCCNC4)ncc3F)n2c1)C(F)(F)F] | Fast-eluting diastereomer- 1,1,1-trifluoro-2-(3-(5- fluoro-2-((R)-piperidin- 3-ylamino)pyrimidin-4- yl)imidazo[1,2-a]pyridin- 6-yl)propan-2-ol | 425.16 |
| 38a | 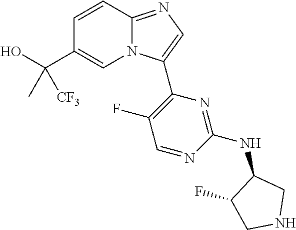 [SMILES: CC(O)(c1ccc2ncc(-c3nc(N[C@H]4CNC[C@@H]4F)ncc3F)n2c1)C(F)(F)F] | Slow-eluting diastereomer-1,1,1- trifluoro-2-(3-(5-fluoro- 2-(((3S,4S)-4- fluoropyrrolidin-3- yl)amino)pyrimidin-4- yl)imidazo[1,2-a]pyridin- 6-yl)propan-2-ol | 429.14 |
| 39a | 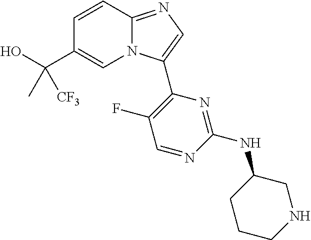 [SMILES: CC(O)(c1ccc2ncc(-c3nc(N[C@@H]4CCCNC4)ncc3F)n2c1)C(F)(F)F] | Slow-eluting diastereomer-1,1,1- trifluoro-2-(3-(5-fluoro- 2-((R)-piperidin-3- ylamino)pyrimidin-4- yl)imidazo[1,2-a]pyridin- 6-yl)propan-2-ol | 425.16 |

### Example 13

### Exemplary Synthetic Procedure #11 (Compounds 40a-42a)

### Compound 40a, 2-(3-(2-(((3S,4S)-4-fluoropiperidin-3-yl)amino)pyrimidin-4-yl)-7-methoxyimidazo[1,2-b]pyridazin-6-yl)propan-2-ol

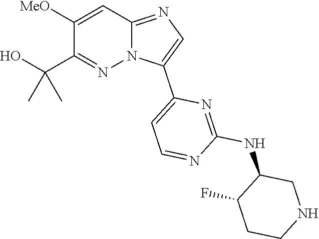 [SMILES: COc1cc2ncc(-c3ccnc(N[C@H]4CNCC[C@@H]4F)n3)n2nc1C(C)(C)O]

### Step A. 6-chloro-4-methoxypyridazin-3-amine

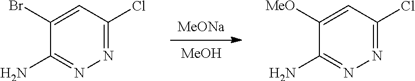 [SMILES: "MeONa" ; "MeOH" ; Nc1nnc(Cl)cc1Br ; COc1cc(Cl)nnc1N]

To a cooled 0° C. solution of 4-bromo-6-chloropyridazin-3-amine (87.00 g, 417.4 mmol) in methanol (900 mL) and ethyl acetate (900 mL) was added sodium methoxide (150.32 g, 834.76 mmol, 30% purity). The resulting reaction was stirred for 3 hours while warming to room temperature. The reaction was then acidified to pH ~3 via addition of acetic acid, then concentrated under reduced pressure. The resulting crude product was triturated three times with 15% methanol in dichloromethane, filtered, and dried under reduced pressure to provide the title compound: LCMS m/z 160.1 [M+H]⁺; ¹H NMR (400 MHz, CD30D) b 6.98 (s, 1H), 4.02-3.90 (s, 3H).

### Step B. 3-bromo-6-chloro-4-methoxypyridazine

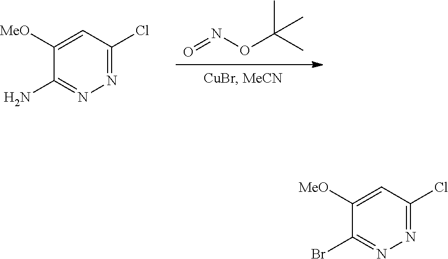

To a cooled 0° C. mixture of 6-chloro-4-methoxypyridazin-3-amine (100.00 g, 626.67 mmol) and copper(I)bromide (152.82 g, 1.07 mol) in acetonitrile (1.50 L) was added tert-butyl nitrite (161.55 g, 1.57 mol). The resulting reaction was stirred for 16 hours while warming to room temperature. The reaction was then diluted with water (1 L) and extracted with ethyl acetate (3×500 mL). The organic extracts were combined, washed with saturated aqueous sodium chloride solution (500 mL), dried over sodium sulfate, filtered, and concentrated under reduced pressure. The resulting crude product was purified by flash chromatography on silica gel (0-70% ethyl acetate in petroleum ether) to provide the title compound: LCMS m/z 225.0 [M+H]⁺; ¹H NMR (400 MHz, CD₃OD) δ 7.45 (s, 1H), 4.14-4.05 (s, 3H).

### Step C. 6-chloro-4-methoxy-3-(prop-1-en-2-yl)pyridazine

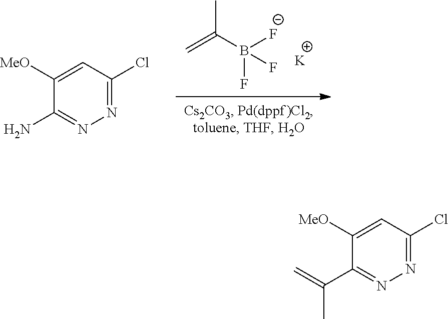

A mixture of 3-bromo-6-chloro-4-methoxypyridazine (30.00 g, 134.3 mmol), potassium isopropenyltrifluoroborate (19.87 g, 134.3 mmol), [1,1-bis(diphenylphosphino)ferrocene]dichloropalladium(II) (9.82 g, 13.4 mmol), and cesium carbonate (131.23 g, 402.77 mmol) in toluene (360 mL), tetrahydrofuran (120 mL) and water (120 mL) was purged with nitrogen, and was then heated at 100° C. for 16 hours under nitrogen atmosphere. The reaction was then cooled to room temperature, diluted with water (500 mL), and extracted with ethyl acetate (3×250 mL). The organic extracts were combined, washed with saturated aqueous sodium chloride solution (300 mL), dried over sodium sulfate, filtered, and concentrated under reduced pressure. The resulting crude product was purified by flash chromatography on silica gel (0-50% ethyl acetate in petroleum ether) to provide the title compound: LCMS m/z 185.2 [M+H]⁺; ¹H NMR (400 MHz, CD₃OD) δ 7.43 (s, 1H), 5.74 (s, 1H), 5.60 (q, J=1.5 Hz, 1H), 4.06-3.95 (s, 3H), 2.24-2.19 (s, 3H).

### Step D. 2-(6-chloro-4-methoxypyridazin-3-yl)propan-2-ol

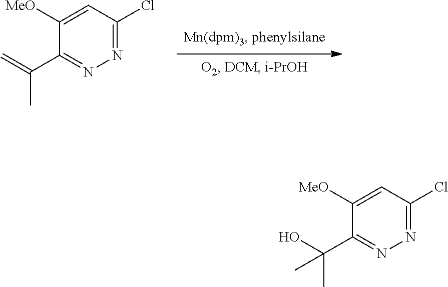

To a solution of 6-chloro-4-methoxy-3-(prop-1-en-2-yl)pyridazine (10.00 g, 54.16 mmol) in isopropanol (300 mL) and dichloromethane (100 mL) was added phenylsilane (17.58 g, 162.49 mmol, 20.05 mL). The resulting reaction was purged with oxygen 3 times and then cooled to 0° C. Tris[(Z)-1-tert-butyl-4,4-dimethyl-3-oxo-pent-1-enoxy]manganese (3.28 g, 5.42 mmol) was added, and the reaction was stirred at 0° C. for 1 hour under oxygen atmosphere. The reaction was then warmed to room temperature, filtered, and concentrated under reduced pressure. The resulting crude product was purified by flash chromatography on silica gel (0-100% ethyl acetate in petroleum ether) to provide the title compound: LCMS m/z 203.0 [M+H]⁺; ¹H NMR (400 MHz, CD₃OD) δ 7.44 (s, 1H), 4.04 (s, 3H), 1.68-1.62 (s, 6H).

### Step E. tert-butyl (6-(2-hydroxypropan-2-yl)-5-methoxypyridazin-3-yl)carbamate

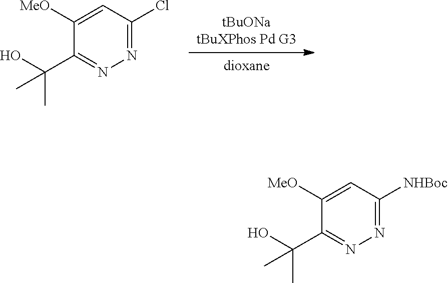 [SMILES: COc1cc(Cl)nnc1C(C)(C)O ; "dioxane" ; "tBuONatBuXPhos Pd G3" ; COc1cc(NC(=O)OC(C)(C)C)nnc1C(C)(C)O]

A mixture of 2-(6-chloro-4-methoxy-pyridazin-3-yl)propan-2-ol (8.00 g, 39.5 mmol), tert-butyl carbamate (13.87 g, 118.4 mmol), [2-(2-aminophenyl)phenyl]-methylsulfonyloxy-palladium ditert-butyl-[2-(2,4,6-triisopropylphenyl)phenyl]phosphane (3.14 g, 3.95 mmol), and sodium tert-butoxide solution (2 M in THF, 39.48 mL) in dioxane (240 mL) was purged with nitrogen, and was then heated at 100° C. for 16 hours under nitrogen atmosphere. The reaction was then cooled to room temperature, diluted with water (200 mL), and extracted with ethyl acetate (3×100 mL). The organic extracts were combined, washed with saturated aqueous sodium chloride solution (100 mL), dried over sodium sulfate, filtered, and concentrated under reduced pressure. The resulting crude product was purified by flash chromatography on silica gel (0-50% ethyl acetate in petroleum ether) to provide the title compound: LCMS m/z 284.0 [M+H]⁺; ¹H NMR (400 MHz, CD₃OD) δ 7.87 (s, 1H), 4.06-3.98 (s, 3H), 1.63 (s, 6H), 1.57 (s, 9H).

### Step F. 2-(6-amino-4-methoxypyridazin-3-yl)propan-2-ol

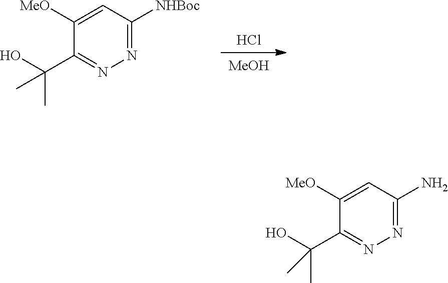 [SMILES: "MeOH" ; COc1cc(NC(=O)OC(C)(C)C)nnc1C(C)(C)O ; Cl ; COc1cc(N)nnc1C(C)(C)O]

A solution of tert-butyl (6-(2-hydroxypropan-2-yl)-5-methoxypyridazin-3-yl) carbamate (10 g, 35.30 mmol) in hydrochloric acid/methanol (3 M, 200 mL) was stirred at room temperature for 16 hours. The reaction was then concentrated under reduced pressure to provide the title compound: LCMS m/z 184.0 [M+H]⁺.

### Step G. 2-(7-methoxyimidazo[1,2-b]pyridazin-6-yl)propan-2-ol

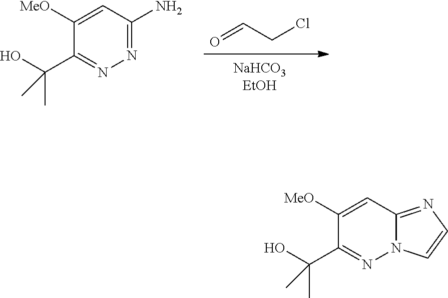

To a solution of 2-chloroacetaldehyde (2.14 g, 10.9 mmol, 1.76 mL, 40% purity) in ethanol (30 mL) were added sodium bicarbonate (0.612 g, 7.28 mmol) and 2-(6-amino-4-methoxypyridazin-3-yl)propan-2-ol (0.800 g, 3.64 mmol, HCl salt). The resulting reaction was stirred at 80° C. for 2 hours. The reaction was then cooled to room temperature, filtered, and concentrated under reduced pressure. The crude product thus obtained was purified by flash chromatography on silica gel (0-100% ethyl acetate in petroleum ether) to provide the title compound: LCMS m/z 208.1 [M+H]⁺.

Step H. 2-(3-iodo-7-methoxyimidazo[1,2-b]pyridazin-6-yl)propan-2-ol

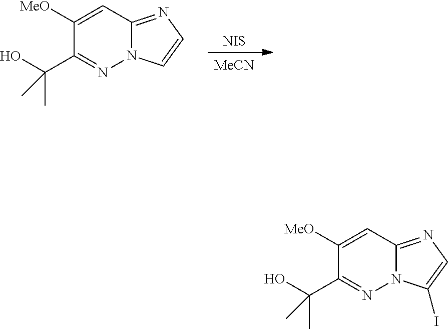

To a cooled 0° C. solution of 2-(7-methoxyimidazo[1,2-b]pyridazin-6-yl)propan-2-ol (0.600 g, 2.90 mmol) in acetonitrile (6 mL) was added 1-iodopyrrolidine-2,5-dione (0.651 g, 2.90 mmol). The resulting reaction was stirred for 2 hours while warming to room temperature. The reaction was then quenched by addition of aqueous sodium hydroxide solution (2 M, 6 mL) and extracted with ethyl acetate (3×5 mL). The organic extracts were combined, washed with saturated aqueous sodium chloride solution (5 mL), dried over sodium sulfate, filtered, and concentrated under reduced pressure to provide the title compound: LCMS m/z 334.0 [M+H]⁺.

### Step I. 2-(3-(2-chloropyrimidin-4-yl)-7-methoxyimidazo[1,2-b]pyridazin-6-yl)propan-2-ol

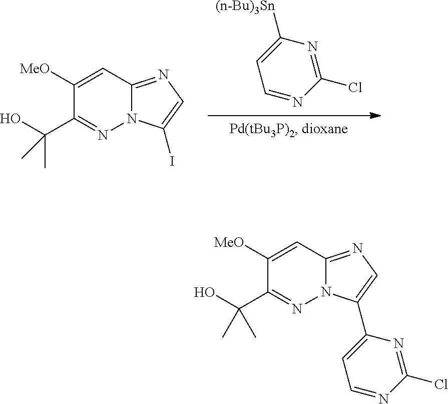

A mixture of 2-(3-iodo-7-methoxyimidazo[1,2-b]pyridazin-6-yl)propan-2-ol (0.200 g, 0.600 mmol), 2-chloro-4-(tributylstannyl)pyrimidine (0.363 g, 0.901 mmol), and palladium tri-tert-butylphosphane (0.031 g, 0.060 mmol) in dioxane (5 mL) was purged with nitrogen, and was then heated at 110° C. for 12 hours under nitrogen atmosphere. The reaction was then cooled to room temperature, filtered, and concentrated under reduced pressure. The resulting crude product was purified by flash chromatography on silica gel (0-80% ethyl acetate in petroleum ether) to provide the title compound: LCMS m/z 320.1 [M+H]⁺.

### Step J. (3S,4S)-tert-butyl 4-fluoro-3-((4-(6-(2-hydroxypropan-2-yl)-7-methoxyimidazo[1,2-b]pyridazin-3-yl)pyrimidin-2-yl)amino)piperidine-1-carboxylate

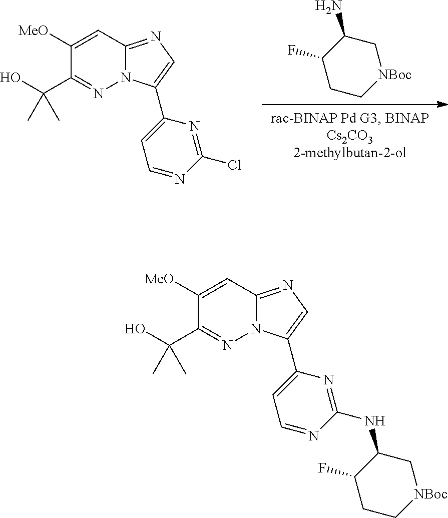

A mixture of 2-(3-(2-chloropyrimidin-4-yl)-7-methoxyimidazo[1,2-b]pyridazin-6-yl)propan-2-ol (0.040 g, 0.125 mmol), (3S,4S)-tert-butyl 3-amino-4-fluoropiperidine-1-carboxylate (0.041 g, 0.188 mmol), methanesulfonato[2,2-bis(diphenylphosphino)-1,1-binaphthyl](2-amino-1,1-biphenyl-2-yl)palladium(II) (0.012 g, 0.013 mmol), (R)-(+)-2,2′-bis(diphenylphosphino)-1,1′-binaphthyl (0.008 g, 0.013 mmol), and cesium carbonate (0.122 g, 0.375 mmol) in 2-methylbutan-2-ol (4 mL) was purged with nitrogen, and was then heated at 80° C. for 4 hours under nitrogen atmosphere. The reaction was then cooled to room temperature, filtered, and concentrated under reduced pressure to provide the title compound: LCMS m/z 502.2 [M+H]⁺.

### Step K. 2-(3-(2-(((3S,4S)-4-fluoropyrrolidin-3-yl)amino)pyrimidin-4-yl)-6-methoxypyrazolo[1,5-a]pyrimidin-5-yl)propan-2-ol

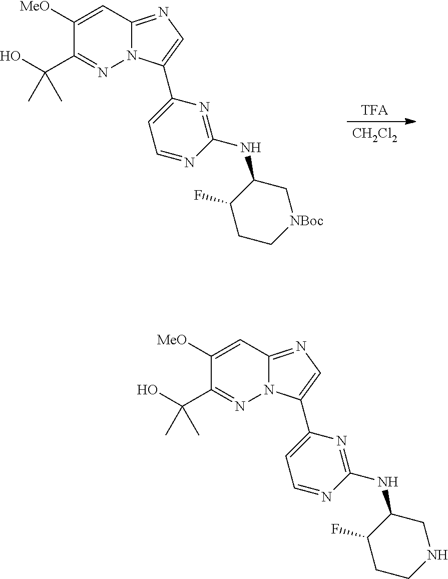 [SMILES: COc1cc2ncc(-c3ccnc(N[C@H]4CN(C(=O)OC(C)(C)C)CC[C@@H]4F)n3)n2nc1C(C)(C)O ; "CH2Cl2" ; "TFA" ; COc1cc2ncc(-c3ccnc(N[C@H]4CNCC[C@@H]4F)n3)n2nc1C(C)(C)O]

To a solution of (3S,4S)-tert-butyl 4-fluoro-3-((4-(6-(2-hydroxypropan-2-yl)-7-methoxyimidazo[1,2-b]pyridazin-3-yl)pyrimidin-2-yl)amino)piperidine-1-carboxylate (0.090 g, 0.179 mol) in dichloromethane (3 mL) was added trifluoroacetic acid (0.50 mL). The resulting reaction was stirred at room temperature for 1 hour, and was then concentrated under reduced pressure. The crude product thus obtained was purified by HPLC (Phenomenex Luna C18 column, 5 micron, 80×30 mm; 1-30% acetonitrile in water containing 0.04% trifluoroacetic acid) to provide the title compound: LCMS m/z 402.1 [M+H]⁺; ¹H NMR (400 MHz, CD₃O) δ 8.63 (s, 1H), 8.51 (d, J=5.5 Hz, 1H), 8.06 (d, J=5.5 Hz, 1H), 7.70 (s, 1H), 5.03-4.97 (m, 1H), 4.62-4.55 (m, 1H), 4.15 (s, 3H), 3.73-3.70 (m, 1H), 3.57-3.49 (m, 1H), 3.29-3.12 (m, 2H), 2.49-2.47 (m, 1H), 2.25-2.01 (m, 1H), 1.79 (s, 6H).

The compounds in Table 12 were all prepared using the synthetic procedures described in Example 13.

TABLE 12
Additional compounds prepared according to Example 13.

| Compound # | Structure | IUPAC Name | LCMS |
|---|---|---|---|
| 41a | 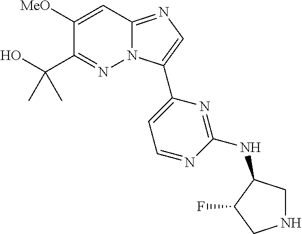 [SMILES: COc1cc2ncc(-c3ccnc(N[C@H]4CNC[C@@H]4F)n3)n2nc1C(C)(C)O] | 2-(3-(2-(((3S,4S)-4- fluoropyrrolidin-3- yl)amino)pyrimidin-4-yl)-7- methoxyimidazo[1,2- b]pyridazin-6-yl)propan-2- ol | 388.1 |
| 42a | 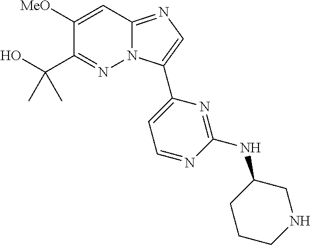 [SMILES: COc1cc2ncc(-c3ccnc(N[C@@H]4CCCNC4)n3)n2nc1C(C)(C)O] | (R)-2-(7-methoxy-3-(2- (piperidin-3- ylamino)pyrimidin-4- yl)imidazo[1,2-b]pyridazin- 6-yl)propan-2-ol | 384.1 |

### Example 14

### Exemplary Synthetic Procedure #12 (Compounds 43a-45a)

### Compound 43a, (R)-2-(3-(5-fluoro-2-(piperidin-3-ylamino)pyrimidin-4-yl)-7-methoxyimidazo[1,2-b]pyridazin-6-yl)propan-2-ol

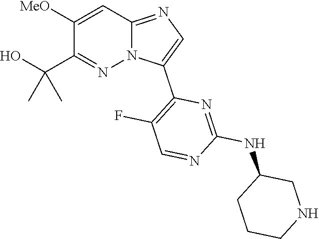 [SMILES: COc1cc2ncc(-c3nc(N[C@@H]4CCCNC4)ncc3F)n2nc1C(C)(C)O]

### Step A. 2-(3-(2-bromo-5-fluoropyrimidin-4-yl)-7-methoxyimidazo[1,2-b]pyridazin-6-yl)propan-2-ol

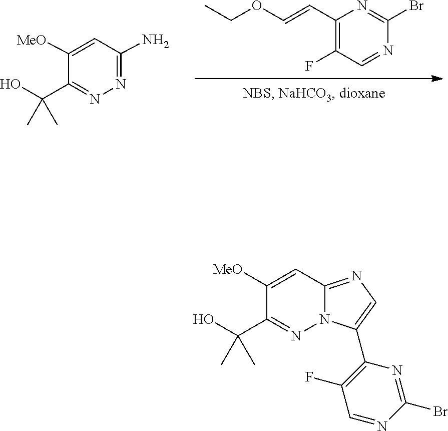

To a cooled 0° C. solution of 2-bromo-4-[(E)-2-ethoxyvinyl]-5-fluoro-pyrimidine (0.647 g, 2.62 mmol) in dioxane (9 mL) and water (3 mL) was added 1-bromopyrrolidine-2,5-dione (0.466 g, 2.62 mmol). The resulting reaction was stirred for 30 minutes while warming to room temperature. 2-(6-Amino-4-methoxy-pyridazin-3-yl)propan-2-ol (0.400 g, 2.18 mmol) and sodium bicarbonate (0.367 g, 4.37 mmol) were then added, and the resulting mixture was heated to 80° C. for 2 hours. The reaction was then cooled to the room temperature, diluted with water (10 mL), and extracted with ethyl acetate (3×5 mL). The organic extracts were combined, washed with saturated aqueous sodium chloride solution (5 mL), dried over anhydrous sodium sulfate, filtered, and concentrated under reduced pressure. The resulting crude product was purified by flash chromatography on silica gel (0-50% ethyl acetate in petroleum ether) to provide the title compound: LCMS m/z 382.0 [M+H]⁺; ¹H NMR (400 MHz, CD₃OD) δ 8.61 (d, J=3.1 Hz, 1H), 8.29 (d, J=2.9 Hz, 1H), 7.51 (s, 1H), 4.06 (s, 3H), 1.75-1.69 (s, 6H).

### Step B. (R)-tert-butyl 3-((5-fluoro-4-(6-(2-hydroxypropan-2-yl)-7-methoxyimidazo[1,2-b]pyridazin-3-yl)pyrimidin-2-yl)amino)piperidine-1-carboxylate

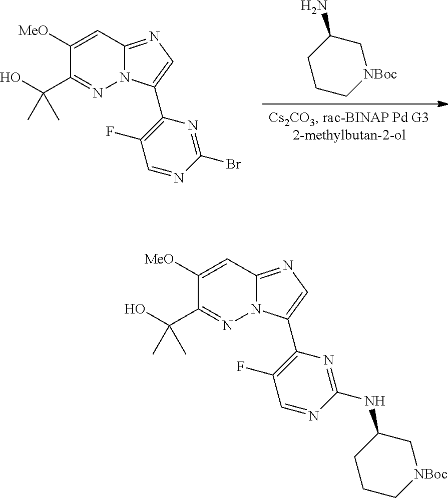

A mixture of 2-(3-(2-bromo-5-fluoropyrimidin-4-yl)-7-methoxyimidazo[1,2-b]pyridazin-6-yl)propan-2-ol (0.050 g, 0.131 mmol), (R)-tert-butyl 3-aminopiperidine-1-carboxylate (0.039 g, 0.196 mmol), methanesulfonato[2,2-bis(diphenylphosphino)-1,1-binaphthyl](2-amino-1,1-biphenyl-2-yl)palladium(II) (0.013 g, 0.013 mmol), (R)-(+)-2,2′-bis(diphenylphosphino)-1,1′-binaphthyl (0.008 g, 0.013 mmol), and cesium carbonate (0.128 g, 0.392 mmol) in 2-methylbutan-2-ol (4 mL) was purged with nitrogen, and was then heated at 80° C. for 4 hours under nitrogen atmosphere. The reaction was then cooled to room temperature, filtered, and concentrated under reduced pressure to provide the title compound: LCMS m/z 502.3 [M+H]⁺.

### Step F. (R)-2-(3-(5-fluoro-2-(piperidin-3-ylamino)pyrimidin-4-yl)-7-methoxyimidazo[1,2-b]pyridazin-6-yl)propan-2-ol

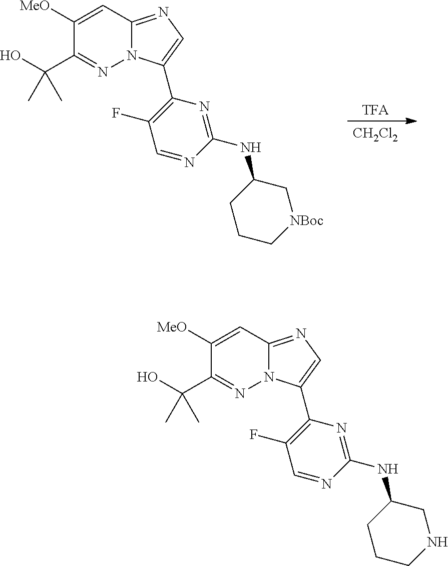 [SMILES: COc1cc2ncc(-c3nc(N[C@@H]4CCCN(C(=O)OC(C)(C)C)C4)ncc3F)n2nc1C(C)(C)O ; "CH2Cl2" ; "TFA" ; COc1cc2ncc(-c3nc(N[C@@H]4CCCNC4)ncc3F)n2nc1C(C)(C)O]

To a solution of (R)-tert-butyl 3-((5-fluoro-4-(6-(2-hydroxypropan-2-yl)-7-methoxyimidazo[1,2-b]pyridazin-3-yl)pyrimidin-2-yl)amino)piperidine-1-carboxylate (0.120 g, 0.239 mmol) in dichloromethane (3 mL) was added trifluoroacetic acid (0.50 mL). The resulting reaction was stirred at room temperature for 1 hour, and was then concentrated under reduced pressure. The crude product thus obtained was purified by HPLC (Phenomenex Luna C18 column, 5 micron, 75×30 mm; 1-30% acetonitrile in water containing 0.04% trifluoroacetic acid) to provide the title compound: LCMS m/z 402.0 [M+H]⁺; ¹H NMR (400 MHz, CD30D) 6 8.42 (d, J=3.1 Hz, 1H), 8.29 (d, J=1.9 Hz, 1H), 7.61 (s, 1H), 4.39-4.24 (m, 1H), 4.12 (s, 3H), 3.70 (br dd, J=3.3, 11.8 Hz, 1H), 3.42-3.35 (m, 1H), 3.09-2.89 (m, 2H), 2.22-2.04 (m, 2H), 1.98-1.75 (m, 2H), 1.73 (d, J=5.0 Hz, 6H).

The compounds in Table 13 were all prepared using the synthetic procedures described in Example 14.

TABLE 13
Additional compounds prepared according to Example 14.

| Compound # | Structure | IUPAC Name | LCMS |
|---|---|---|---|
| 44a | 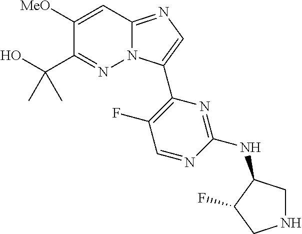 [SMILES: COc1cc2ncc(-c3nc(N[C@H]4CNC[C@@H]4F)ncc3F)n2nc1C(C)(C)O] | 2-(3-(5-fluoro-2- (((3S,4S)-4- fluoropyrrolidin-3- yl)amino)pyrimidin-4- yl)-7- methoxyimidazo[1,2- b]pyridazin-6-yl)propan- 2-ol | 406.1 |
| 45a | 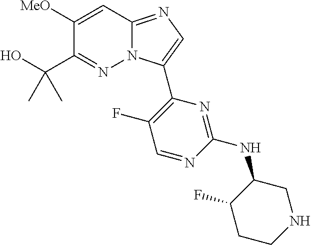 [SMILES: COc1cc2ncc(-c3nc(N[C@H]4CNCC[C@@H]4F)ncc3F)n2nc1C(C)(C)O] | 2-(3-(5-fluoro-2- (((3S,4S)-4- fluoropiperidin-3- yl)amino)pyrimidin-4- yl)-7- methoxyimidazo[1,2- b]pyridazin-6-yl)propan- 2-ol | 420.1 |

### Example 15

### Exemplary Synthetic Procedure #13 (Compounds 46a-48a)

### Compound 46a, 2-(3-(5-fluoro-2-(((3S,4S)-4-fluoropiperidin-3-yl)amino)pyrimidin-4-yl)imidazo[1,2-a]pyridin-6-yl)propan-2-ol

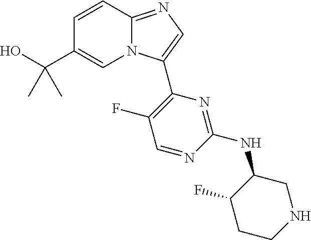 [SMILES: CC(C)(O)c1ccc2ncc(-c3nc(N[C@H]4CNCC[C@@H]4F)ncc3F)n2c1]

### Step A. 5-(prop-1-en-2-yl)pyridin-2-amine

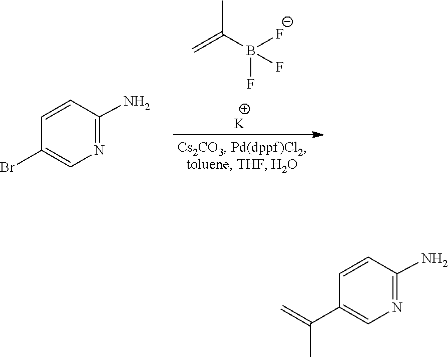

A mixture of 5-bromopyridin-2-amine (3.00 g, 17.3 mmol), potassium isopropenyltrifluoroborate (3.08 g, 20.8 mmol), cesium carbonate (16.95 g, 52.02 mmol), and [1,1-bis(diphenylphosphino)ferrocene]dichloropalladium(II) (1.27 g, 1.73 mmol) in toluene (30 mL), tetrahydrofuran (10 mL), and water (10 mL) was purged with nitrogen, and was then heated at 100° C. for 16 hours under nitrogen atmosphere. The reaction was cooled to room temperature, filtered, diluted with water (40 mL), and extracted with ethyl acetate (3×40 mL). The organic extracts were combined, washed with saturated aqueous sodium chloride solution (40 mL), dried over sodium sulfate, filtered, and concentrated under reduced pressure. The resulting crude product was purified by flash chromatography on silica gel (15-50% ethyl acetate in petroleum ether) to provide the title compound: LCMS m/z 135.05 [M+H]⁺; ¹H NMR (400 MHz, CD₃OD) δ 7.98 (d, J=2.4 Hz, 1H), 7.64 (dd, J=2.4 Hz 8.8 Hz, 1H), 6.55 (d, J=8.8 Hz, 1H), 5.24 (s, 1H), 4.93 (s, 1H), 2.07 (s, 3H).

### Step B. 6-(2,2-difluoroethoxy)pyrazolo[1,5-a]pyrimidine

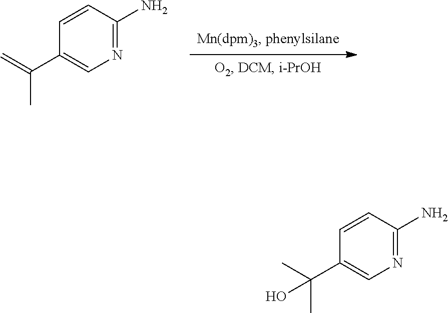

A mixture of 5-isopropenylpyridin-2-amine (0.500 g, 3.73 mmol) and phenylsilane (2.02 g, 18.63 mmol, 2.30 mL) in propan-2-ol (30 mL) and dichloromethane (10 mL) was purged with oxygen 3 times, then cooled to 0° C. Tris[(Z)-1-tert-butyl-4,4-dimethyl-3-oxo-pent-1-enoxy]manganese (0.225 g, 0.373 mol) was added, and the resulting reaction was stirred at 0° C. for 4 hours under oxygen atmosphere. The reaction was then diluted with water (40 mL) and extracted with ethyl acetate (3×40 mL). The organic extracts were combined, washed with saturated aqueous sodium chloride solution (40 mL), dried over sodium sulfate, filtered, and concentrated under reduced pressure. The resulting crude product was purified by flash chromatography on silica gel (30-70% ethyl acetate in petroleum ether) to provide the title compound: LCMS m/z 153.10 [M+H]⁻.

### Step C. 2-(3-(2-bromo-5-fluoropyrimidin-4-yl)imidazo[1,2-a]pyridin-6-yl)propan-2-ol

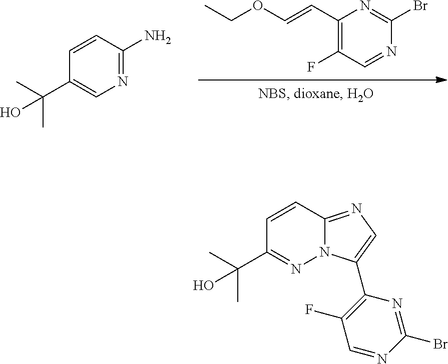

To a cooled 0° C. solution of 2-bromo-4-[(E)-2-ethoxyvinyl]-5-fluoro-pyrimidine (0.235 g, 0.950 mmol) in dioxane (1.5 mL) and water (0.5 mL) was added 1-bromopyrrolidine-2,5-dione (0.185 g, 1.04 mmol). The resulting reaction was stirred for 30 minutes while warming to room temperature. 2-(6-Amino-3-pyridyl)propan-2-ol (0.230 g, 0.864 mmol, TFA salt) was added, and the reaction was then heated to 80° C. for 5 hours. The reaction was then cooled to room temperature, basified to pH ~8 by addition of aqueous sodium hydroxide solution (2 M, 5 mL), and stirred for 30 minutes. The reaction was then diluted with water (5 mL) and extracted with ethyl acetate (3×5 mL). The organic extracts were combined, washed with saturated aqueous sodium chloride solution (10 mL), dried over sodium sulfate, filtered, and concentrated under reduced pressure. The resulting crude product was purified by flash chromatography on silica gel (5-40% ethyl acetate in petroleum ether) to provide the title compound: LCMS m/z 352.9 [M+H]⁺; ¹H NMR (400 MHz, CD₃OD) δ 10.18 (s, 1H), 8.59 (d, J=3.6 Hz, 1H), 8.53 (d, J=4.2 Hz, 1H), 7.86 (dd, J=1.8, 9.4 Hz, 1H), 7.77 (d, J=9.4 Hz, 1H), 1.67 (s, 6H).

### Step D. tert-butyl (3S, 4S)-4-fluoro-3-((5-fluoro-4-(6-(2-hydroxypropan-2-yl)imidazo [1,2-a]pyridin-3-yl)pyrimidin-2-yl)amino)piperidine-1-carboxylate

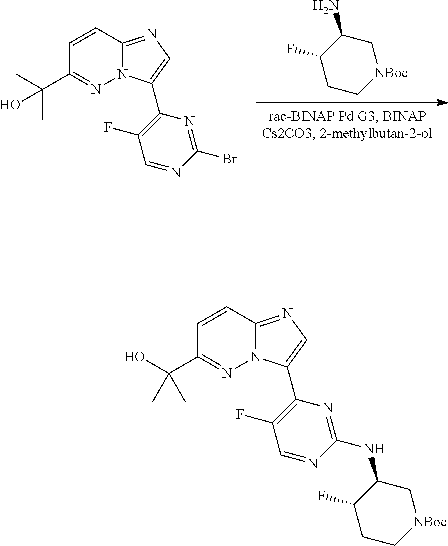

A mixture of 2-[3-(2-bromo-5-fluoro-pyrimidin-4-yl)imidazo[1,2-a]pyridin-6-yl]propan-2-ol (0.050 g, 0.142 mmol), tert-butyl (3S,4S)-3-amino-4-fluoro-piperidine-1-carboxylate (0.047 g, 0.214 mmol), cesium carbonate (0.139 g, 0.427 mol), (R)-(+)-2,2′-bis(diphenylphosphino)-1,1′-binaphthyl (0.013 g, 0.021 mmol), and methanesulfonato[2,2-bis(diphenylphosphino)-1,1-binaphthyl](2-amino-1,1-biphenyl-2-yl)palladium(II) (0.014 g, 0.014 mmol) in 2-methylbutan-2-ol (2 mL) was purged with nitrogen, and was then heated at 80° C. for 4 hours under nitrogen atmosphere. The reaction was then cooled to room temperature, filtered, and concentrated under reduced pressure to provide the title compound: LCMS m/z 489.3 [M+H]⁺.

### Step E. 2-(3-(5-fluoro-2-(((3S,4S)-4-fluoropiperidin-3-yl)amino)pyrimidin-4-yl)imidazo[1,2-a]pyridin-6-yl)propan-2-ol

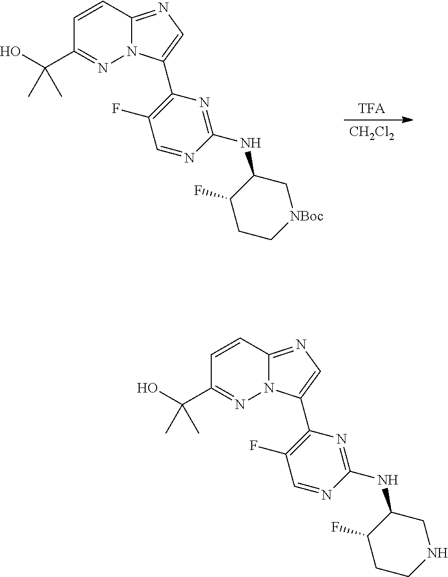 [SMILES: CC(C)(C)OC(=O)N1CC[C@H](F)[C@@H](Nc2ncc(F)c(-c3cnc4ccc(C(C)(C)O)nn34)n2)C1 ; "CH2Cl2" ; "TFA" ; CC(C)(O)c1ccc2ncc(-c3nc(N[C@H]4CNCC[C@@H]4F)ncc3F)n2n1]

A solution of tert-butyl (3S,4S)-4-fluoro-3-[[5-fluoro-4-[6-(1-hydroxy-1-methyl-ethyl)imidazo[1,2-a]pyridin-3-yl]pyrimidin-2-yl]amino]piperidine-1-carboxylate (0.050 g, 0.102 mmol) in dichloromethane (2.0 mL) and trifluoroacetic acid (0.50 mL) was stirred at room temperature for 1 hour, then concentrated under reduced pressure. The resulting crude product was purified by HPLC (Phenomenex Luna C18 column, 3 micron, 75×30 mm; 1-30% acetonitrile in water containing 0.1% trifluoroacetic acid) to provide the title compound: LCMS m/z 389.05 [M+H]⁺; ¹H NMR (400 MHz, CD₃OD) δ 10.42 (s, 1H), 8.64 (d, J=2.8 Hz, 1H), 8.47 (d, J=3.5 Hz, 1H), 8.09-7.88 (m, 2H), 4.98-4.96 (m, 1H), 4.54-4.50 (m, 1H), 3.83-3.80 (m, 1H), 3.63-3.48 (m, 1H), 3.29-3.08 (m, 1H), 3.30-3.05 (m, 1H), 2.51-2.48 (m, 1H), 2.26-2.01 (m, 1H), 1.69 (d, J=19.5 Hz, 6H).

The compound in Table 14 were all prepared using the synthetic procedures described in Example 15.

TABLE 14
Additional compounds prepared according to Example 15.

| Compound # | Structure | IUPAC Name | LCMS |
|---|---|---|---|
| 47a | 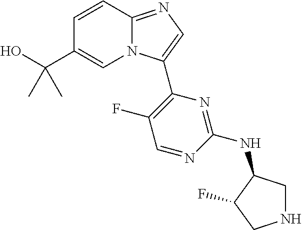 [SMILES: CC(C)(O)c1ccc2ncc(-c3nc(N[C@H]4CNC[C@@H]4F)ncc3F)n2c1] | 2-(3-(5-fluoro-2- (((3S,4S)-4- fluoropyrrolidin-3- yl)amino)pyrimidin-4- yl)imidazo[1,2-a]pyridin- 6-yl)propan-2-ol | 375.17 |
| 48a | 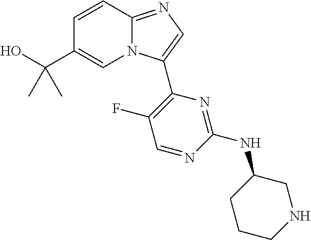 [SMILES: CC(C)(O)c1ccc2ncc(-c3nc(N[C@@H]4CCCNC4)ncc3F)n2c1] | (R)-2-(3-(5-fluoro-2- (piperidin-3- ylamino)pyrimidin-4- yl)imidazo[1,2-a]pyridin- 6-yl)propan-2-ol | 371.1 |

### Example 16

### Exemplary Synthetic Procedure #14 (Compounds 49a-70a)

### Compound 49a, 4-(6-(3,3-difluorocyclobutyl)-7-methoxyimidazo[1,2-b]pyridazin-3-yl)-5-fluoro-N-((3S,4S)-4-fluoropiperidin-3-yl)pyrimidin-2-amine

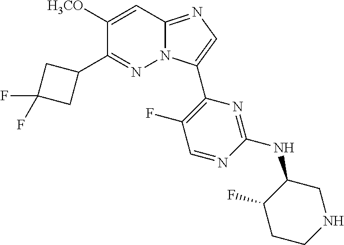 [SMILES: COc1cc2ncc(-c3nc(N[C@H]4CNCC[C@@H]4F)ncc3F)n2nc1C1CC(F)(F)C1]

### Step A. 6-chloro-3-(3,3-difluorocyclobutyl)-4-methoxypyridazine

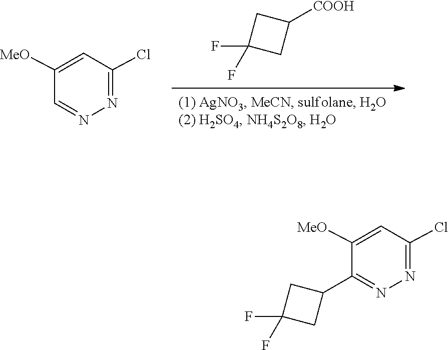 [SMILES: COc1cnnc(Cl)c1 ; "(1) AgNO3, MeCN, sulfolane, H2O(2) H2SO4, NH4S2O8, H2O" ; O=C(O)C1CC(F)(F)C1 ; COc1cc(Cl)nnc1C1CC(F)(F)C1]

To a solution of 3-chloro-5-methoxy-pyridazine (10.00 g, 69.18 mmol) in sulfolane (20 mL), water (100 mL), and acetonitrile (20 mL) were added 3,3-difluorocyclobutanecarboxylic acid (18.83 g, 138.4 mmol) and silver nitrate (11.75 g, 69.18 mmol). The resulting reaction was heated to 50° C. for 30 minutes. A solution of sulfuric acid (20.35 g, 207.5 mmol, 11.06 mL) in water (30 mL) was then added, followed by dropwise addition of a solution ammonia sulfoxy hydrogen sulfate (26.78 g, 117.33 mmol, 25.50 mL) in water (30 mL) over 40 minutes. The reaction was heated to 70° C. for 20 minutes, and was then cooled to room temperature and stirred for 24 hours. The reaction was then neutralized to pH ~7 by addition of aqueous sodium hydroxide solution (30% w/w, 70 mL), diluted with water (100 mL), and extracted with ethyl acetate (3×100 mL). The organic extracts were combined, washed with saturated aqueous sodium chloride solution (3×100 mL), dried over anhydrous sodium sulfate, filtered, and concentrated under reduced pressure. The resulting crude product was purified by flash chromatography on silica gel (0-70% ethyl acetate in petroleum ether) to provide the title compound: LCMS m/z 235.04 [M+H]⁺. ¹H NMR (400 MHz, CD₃OD) δ 7.35 (s, 1H), 4.00 (s, 3H), 3.99-3.69 (m, 1H), 3.07-2.91 (m, 4H).

### Step B. tert-butyl (6-(3,3-difluorocyclobutyl)-5-methoxypyridazin-3-yl)carbamate

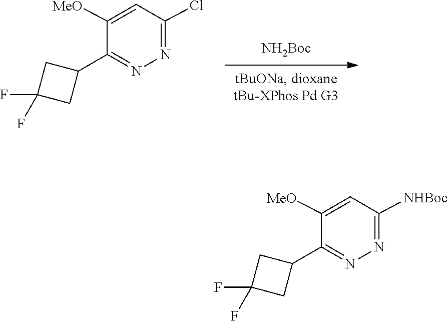 [SMILES: COc1cc(Cl)nnc1C1CC(F)(F)C1 ; "tBuONa, dioxanetBu-XPhos Pd G3" ; COc1cc(NC(=O)OC(C)(C)C)nnc1C1CC(F)(F)C1 ; "NH2Boc"]

A mixture of 6-chloro-3-(3,3-difluorocyclobutyl)-4-methoxy-pyridazine (0.800 g, 3.41 mmol), tert-butyl carbamate (1.20 g, 10.2 mmol), sodium 2-methylpropan-2-olate (2 M in THF, 3.41 mL), and [2-(2-aminophenyl)phenyl]-methylsulfonyloxy-palladium ditert-butyl-[2-(2,4,6-triisopropylphenyl)phenyl]phosphane (0.271 g, 0.341 mmol) in dioxane (20 mL) was purged with nitrogen, and was then heated at 100° C. for 16 hours under nitrogen atmosphere. The reaction was then cooled to room temperature, diluted with water (30 mL), and extracted with ethyl acetate (3×20 mL). The organic extracts were combined, washed with saturated aqueous sodium chloride solution (30 mL), dried over sodium sulfate, filtered, and concentrated under reduced pressure. The resulting crude product was purified by flash chromatography on silica gel (0-80% ethyl acetate in petroleum ether) to provide the title compound: LCMS m/z 316.2 [M+H]⁺; ¹H NMR (400 MHz, CD₃OD) δ 7.78 (s, 1H), 3.97 (s, 3H), 3.96-3.65 (m, 1H), 3.03-2.88 (m, 4H), 1.55 (s, 9H).

### Step C. 6-(3,3-difluorocyclobutyl)-5-methoxypyridazin-3-amine

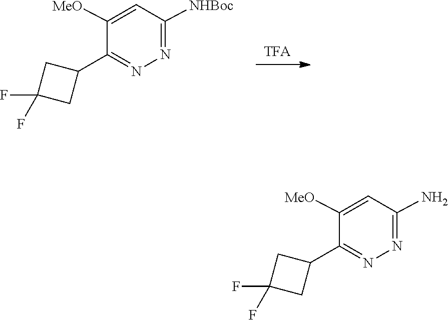 [SMILES: COc1cc(NC(=O)OC(C)(C)C)nnc1C1CC(F)(F)C1 ; COc1cc(N)nnc1C1CC(F)(F)C1 ; "TFA"]

A solution of tert-butyl N-[6-(3,3-difluorocyclobutyl)-5-methoxy-pyridazin-3-yl]carbamate (1.10 g, 3.49 mmol) in trifluoroacetic acid (10 mL) was stirred at room temperature for 1 hour, and was then concentrated under reduced pressure to provide the title compound: LCMS m/z 216.09 [M+H]⁺.

### Step D. 3-(2-bromo-5-fluoropyrimidin-4-yl)-6-(3,3-difluorocyclobutyl)-7-methoxyimidazo[1,2-b]pyridazine

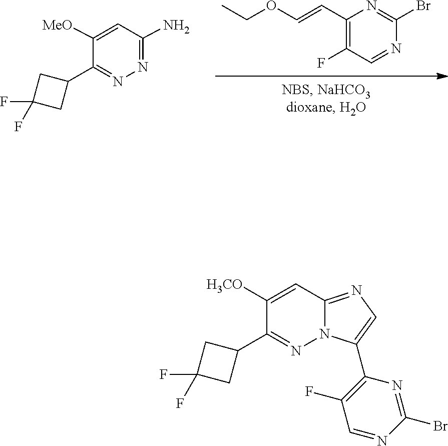

To a cooled 0° C. solution of 2-bromo-4-[(E)-2-ethoxyvinyl]-5-fluoro-pyrimidine (0.661 g, 2.68 mmol) in dioxane (9 mL) and water (3 mL) was added 1-bromopyrrolidine-2,5-dione (0.476 g, 2.68 mmol). The resulting reaction was stirred for 30 minutes while warming to room temperature. 6-(3,3-Difluorocyclobutyl)-5-methoxy-pyridazin-3-amine (0.480 g, 2.23 mmol) and sodium bicarbonate (0.562 g, 6.69 mmol) were then added, and the reaction was heated to 80° C. for 5 hours. The reaction was then cooled to room temperature, diluted with water (15 mL), and extracted with ethyl acetate (3×15 mL). The organic extracts were combined, washed with saturated aqueous sodium chloride solution (15 mL), dried over sodium sulfate, filtered, and concentrated under reduced pressure. The resulting crude product was purified by flash chromatography on silica gel (50-90% ethyl acetate in petroleum ether) to provide the title compound: LCMS m/z 414.01 [M+H]⁺; ¹H NMR (400 MHz, CD₃OD) δ 8.64 (d, J=3.1 Hz, 1H), 8.30 (d, J=2.8 Hz, 1H), 7.46 (s, 1H), 4.07-4.02 (s, 3H), 3.84-3.72 (m, 1H), 3.23-3.10 (m, 2H), 3.04-2.97 (m, 2H).

### Step E. tert-butyl (6-(3,3-difluorocyclobutyl)-5-methoxypyridazin-3-yl)carbamate

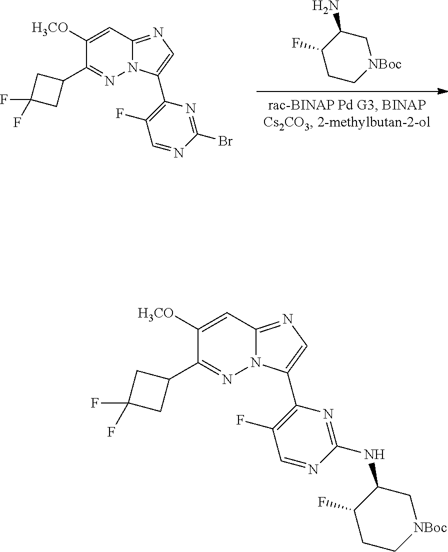

A mixture of 3-(2-bromo-5-fluoro-pyrimidin-4-yl)-6-(3,3-difluorocyclobutyl)-7-methoxy-imidazo[1,2-b]pyridazine (0.070 g, 0.169 mmol), tert-butyl (3S,4S)-3-amino-4-fluoro-piperidine-1-carboxylate (0.055 g, 0.254 mmol), [2-(2-aminophenyl)phenyl]-methylsulfonyloxy-palladium[1-(2-diphenylphosphanyl-1-naphthyl)-2-naphthyl]-diphenyl-phosphane (0.017 g, 0.017 mmol), (R)-(+)-2,2′-bis(diphenylphosphino)-1,1′-binaphthyl (0.016 g, 0.025 mmol), and cesium carbonate (0.165 g, 0.507 mmol) in 2-methylbutan-2-ol (3 mL) was purged with nitrogen, and was then heated at 80° C. for 2 hours under nitrogen atmosphere. The reaction was then cooled to room temperature, filtered, and concentrated under reduced pressure to provide the title compound: LCMS m/z 552.2 [M+H]⁺.

### Step F. 4-(6-(3,3-difluorocyclobutyl)-7-methoxyimidazo[1,2-b]pyridazin-3-yl)-5-fluoro-N-((3S,4S)-4-fluoropiperidin-3-yl)pyrimidin-2-amine

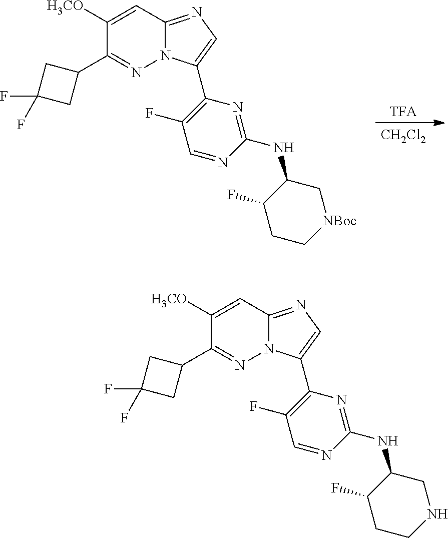 [SMILES: COc1cc2ncc(-c3nc(N[C@H]4CN(C(=O)OC(C)(C)C)CC[C@@H]4F)ncc3F)n2nc1C1CC(F)(F)C1 ; "CH2Cl2" ; COc1cc2ncc(-c3nc(N[C@H]4CNCC[C@@H]4F)ncc3F)n2nc1C1CC(F)(F)C1 ; "TFA"]

To a solution of tert-butyl (3S,4S)-3-[[4-[6-(3,3-difluorocyclobutyl)-7-methoxy-imidazo[1,2-b]pyridazin-3-yl]-5-fluoro-pyrimidin-2-yl]amino]-4-fluoro-piperidine-1-carboxylate (0.230 g, 0.417 mmol) in dichloromethane (3 mL) was added trifluoroacetic acid (1 mL). The resulting reaction was stirred at room temperature for 1 hour, and was then concentrated under reduced pressure. The crude product thus obtained was purified by HPLC (Phenomenex Luna C18 column, 3 micron, 75×30 mm; 10-40% o acetonitrile in water containing 0.100 trifluoroacetic acid) to provide the title compound: LCMS m/z 452.0 [M+H]⁺. ¹H NMR (400 MHz, CD₃OD) δ 8.48 (d, J=2.5 Hz, 1H), 8.20 (s, 1H), 7.54 (s, 1H), 4.94-4.82 (m, 1H), 4.47-4.46 (m, 1H), 4.08 (s, 3H), 3.79-3.77 (m, 1H), 3.62-3.59 (m, 1H), 3.49-3.41 (m, 1H), 3.21-3.18 (m, 2H), 3.06-2.94 (m, 4H), 2.44-2.42 (m, 1H), 2.09-2.06 (m, 1H).

The compounds in Table 15 were all prepared using the synthetic procedures described in Example 16.

TABLE 15
Additional compounds prepared according to Example 16.

| Compound # | Structure | IUPAC Name | LCMS |
|---|---|---|---|
| 50a | 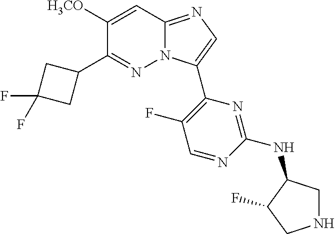 [SMILES: COc1cc2ncc(-c3nc(N[C@H]4CNC[C@@H]4F)ncc3F)n2nc1C1CC(F)(F)C1] | 4-(6-(3,3- difluorocyclobutyl)-7- methoxyimidazo[1,2- b]pyridazin-3-yl)-5-fluoro- N-((3S,4S)-4- fluoropyrrolidin-3- yl)pyrimidin-2-amine | 438.16 |
| 51a | 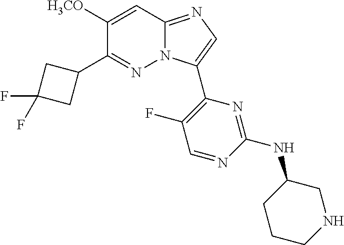 [SMILES: COc1cc2ncc(-c3nc(N[C@@H]4CCCNC4)ncc3F)n2nc1C1CC(F)(F)C1] | (R)-4-(6-(3,3- difluorocyclobutyl)-7- methoxyimidazo[1,2- b]pyridazin-3-yl)-5-fluoro- N-(piperidin-3- yl)pyrimidin-2-amine | 434.18 |
| 52a | 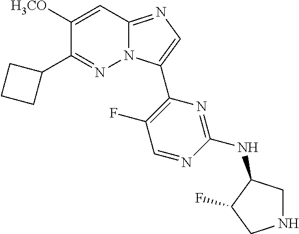 [SMILES: COc1cc2ncc(-c3nc(N[C@H]4CNC[C@@H]4F)ncc3F)n2nc1C1CCC1] | 4-(6-cyclobutyl-7- methoxyimidazo[1,2- b]pyridazin-3-yl)-5-fluoro- N-((3S,4S)-4- fluoropyrrolidin-3- yl)pyrimidin-2-amine | 401.2 |
| 53a | 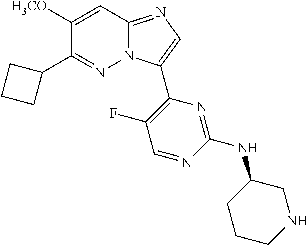 [SMILES: COc1cc2ncc(-c3nc(N[C@@H]4CCCNC4)ncc3F)n2nc1C1CCC1] | (R)-4-(6-cyclobutyl-7- methoxyimidazo[1,2- b]pyridazin-3-yl)-5-fluoro- N-(piperidin-3- yl)pyrimidin-2-amine | 398.1 |
| 54a | 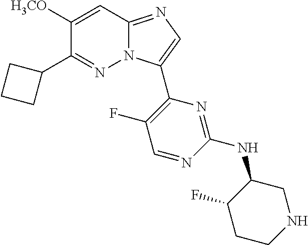 [SMILES: COc1cc2ncc(-c3nc(N[C@H]4CNCC[C@@H]4F)ncc3F)n2nc1C1CCC1] | 4-(6-cyclobutyl-7- methoxyimidazo[1,2- b]pyridazin-3-yl)-5-fluoro- N-((3S,4S)-4- fluoropiperidin-3- yl)pyrimidin-2-amine | 415.2 |
| 55a | 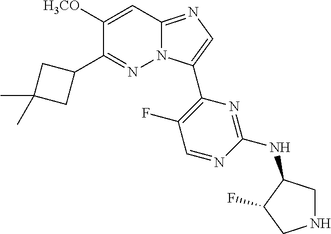 [SMILES: COc1cc2ncc(-c3nc(N[C@H]4CNC[C@@H]4F)ncc3F)n2nc1C1CC(C)(C)C1] | 4-(6-(3,3- dimethylcyclobutyl)-7- methoxyimidazo[1,2- b]pyridazin-3-yl)-5-fluoro- N-((3S,4S)-4- fluoropyrrolidin-3- yl)pyrimidin-2-amine | 430.1 |
| 56a | 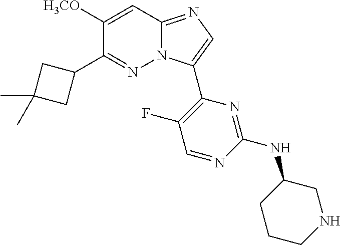 [SMILES: COc1cc2ncc(-c3nc(N[C@@H]4CCCNC4)ncc3F)n2nc1C1CC(C)(C)C1] | (R)-4-(6-(3,3- dimethylcyclobutyl)-7- methoxyimidazo[1,2- b]pyridazin-3-yl)-5-fluoro- N-(piperidin-3- yl)pyrimidin-2-amine | 426.1 |
| 57a | 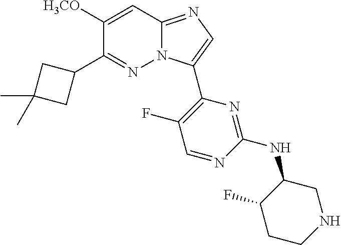 [SMILES: COc1cc2ncc(-c3nc(N[C@H]4CNCC[C@@H]4F)ncc3F)n2nc1C1CC(C)(C)C1] | 4-(6-(3,3- dimethylcyclobutyl)-7- methoxyimidazo[1,2- b]pyridazin-3-yl)-5-fluoro- N-((3S,4S)-4- fluoropiperidin-3- yl)pyrimidin-2-amine | 444.1 |
| 58a | 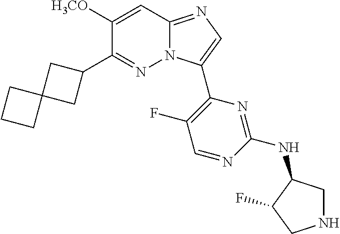 [SMILES: COc1cc2ncc(-c3nc(N[C@H]4CNC[C@@H]4F)ncc3F)n2nc1C1CC2(CCC2)C1] | 5-fluoro-N-((3S,4S)-4- fluoropyrrolidin-3-yl)-4- (7-methoxy-6- (spiro[3.3]heptan-2- yl)imidazo[1,2- b]pyridazin-3- yl)pyrimidin-2-amine | 442.1 |
| 59a | 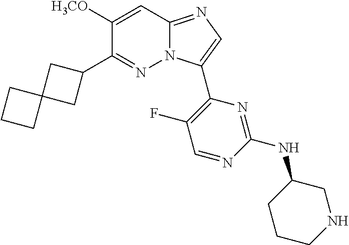 [SMILES: COc1cc2ncc(-c3nc(N[C@@H]4CCCNC4)ncc3F)n2nc1C1CC2(CCC2)C1] | (R)-5-fluoro-4-(7- methoxy-6- (spiro[3.3]heptan-2- yl)imidazo[1,2- b]pyridazin-3-yl)-N- (piperidin-3-yl)pyrimidin- 2-amine | 438.1 |
| 60a | 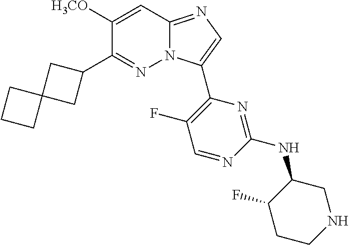 [SMILES: COc1cc2ncc(-c3nc(N[C@H]4CNCC[C@@H]4F)ncc3F)n2nc1C1CC2(CCC2)C1] | 5-fluoro-N-((3S,4S)-4- fluoropiperidin-3-yl)-4-(7- methoxy-6- (spiro[3.3]heptan-2- yl)imidazo[1,2- b]pyridazin-3- yl)pyrimidin-2-amine | 456.1 |
| 61a | 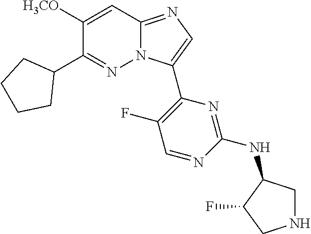 [SMILES: COc1cc2ncc(-c3nc(N[C@H]4CNC[C@@H]4F)ncc3F)n2nc1C1CCCC1] | 4-(6-cyclopentyl-7- methoxyimidazo[1,2- b]pyridazin-3-yl)-5-fluoro- N-((3S,4S)-4- fluoropyrrolidin-3- yl)pyrimidin-2-amine | 416.1 |
| 62a | 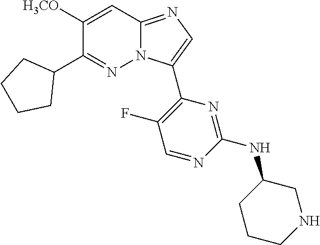 [SMILES: COc1cc2ncc(-c3nc(N[C@@H]4CCCNC4)ncc3F)n2nc1C1CCCC1] | (R)-4-(6-cyclopentyl-7- methoxyimidazo[1,2- b]pyridazin-3-yl)-5-fluoro- N-(piperidin-3- yl)pyrimidin-2-amine | 412.1 |
| 63a | 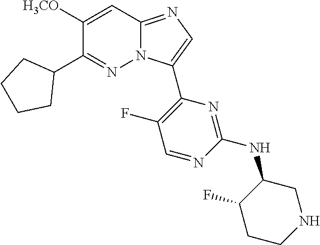 [SMILES: COc1cc2ncc(-c3nc(N[C@H]4CNCC[C@@H]4F)ncc3F)n2nc1C1CCCC1] | 4-(6-cyclopentyl-7- methoxyimidazo[1,2- b]pyridazin-3-yl)-5-fluoro- N-((3S,4S)-4- fluoropiperidin-3- yl)pyrimidin-2-amine | 430.1 |
| 64a | 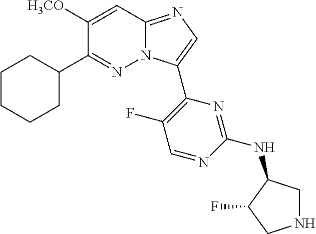 [SMILES: COc1cc2ncc(-c3nc(N[C@H]4CNC[C@@H]4F)ncc3F)n2nc1C1CCCCC1] | 4-(6-cyclohexyl-7- methoxyimidazo[1,2- b]pyridazin-3-yl)-5-fluoro- N-((3S,4S)-4- fluoropyrrolidin-3- yl)pyrimidin-2-amine | 430.1 |
| 65a | 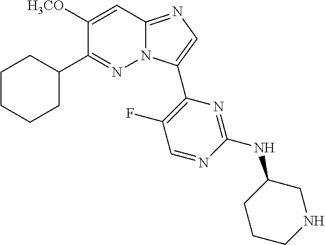 [SMILES: COc1cc2ncc(-c3nc(N[C@@H]4CCCNC4)ncc3F)n2nc1C1CCCCC1] | (R)-4-(6-cyclohexyl-7- methoxyimidazo[1,2- b]pyridazin-3-yl)-5-fluoro- N-(piperidin-3- yl)pyrimidin-2-amine | 426.1 |
| 66a | 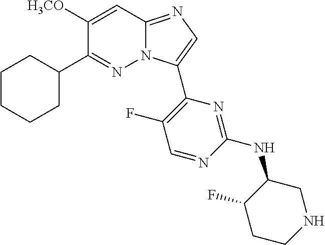 [SMILES: COc1cc2ncc(-c3nc(N[C@H]4CNCC[C@@H]4F)ncc3F)n2nc1C1CCCCC1] | 4-(6-cyclohexyl-7- methoxyimidazo[1,2- b]pyridazin-3-yl)-5-fluoro- N-((3S,4S)-4- fluoropiperidin-3- yl)pyrimidin-2-amine | 444.1 |
| 67a | 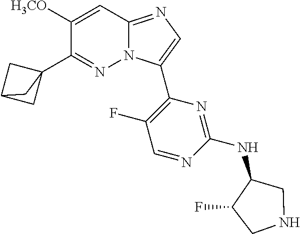 [SMILES: COc1cc2ncc(-c3nc(N[C@H]4CNC[C@@H]4F)ncc3F)n2nc1C12CC(C1)C2] | 4-(6- (bicyclo[1.1.1]pentan-1- yl)-7-methoxyimidazo[1,2- b]pyridazin-3-yl)-5-fluoro- N-((3S,4S)-4- fluoropyrrolidin-3- yl)pyrimidin-2-amine | 414.1 |
| 68a | 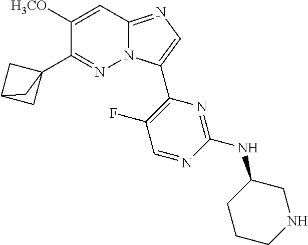 [SMILES: COc1cc2ncc(-c3nc(N[C@@H]4CCCNC4)ncc3F)n2nc1C12CC(C1)C2] | (R)-4-(6- (bicyclo[1.1.1]pentan-1- yl)-7-methoxyimidazo[1,2- b]pyridazin-3-yl)-5-fluoro- N-(piperidin-3- yl)pyrimidin-2-amine | 410.1 |
| 69a | 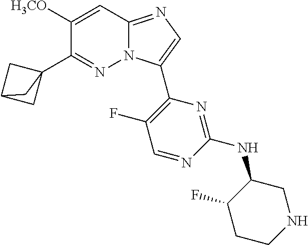 [SMILES: COc1cc2ncc(-c3nc(N[C@H]4CNCC[C@@H]4F)ncc3F)n2nc1C12CC(C1)C2] | 4-(6- (bicyclo[1.1.1]pentan-1- yl)-7-methoxyimidazo[1,2- b]pyridazin-3-yl)-5-fluoro- N-((3S,4S)-4- fluoropiperidin-3- yl)pyrimidin-2-amine | 428.1 |
| 70a | 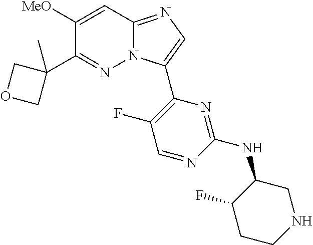 [SMILES: COc1cc2ncc(-c3nc(N[C@H]4CNCC[C@@H]4F)ncc3F)n2nc1C1(C)COC1] | 5-fluoro-N-((3S,4S)-4- fluoropiperidin-3-yl)-4-(7- methoxy-6-(3- methyloxetan-3- yl)imidazo[1,2- b]pyridazin-3- yl)pyrimidin-2-amine | 432.2 |

### Example 17

### Exemplary Synthetic Procedure #15 (71a-73a)

### Compound 71a, 6-(6-(3,3-difluorocyclobutyl)-7-methoxyimidazo[1,2-b]pyridazin-3-yl)-N-((3S,4S)-4-fluoropyrrolidin-3-yl)pyrazin-2-amine

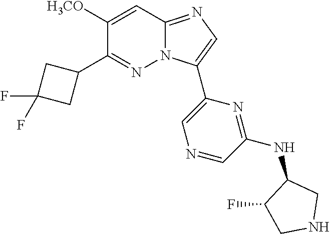 [SMILES: COc1cc2ncc(-c3cncc(N[C@H]4CNC[C@@H]4F)n3)n2nc1C1CC(F)(F)C1]

### Step A. 3-(6-bromopyrazin-2-yl)-6-3,3-difluorocyclobutyl)-7-methoxyimidazo[1,2-]pyridazine

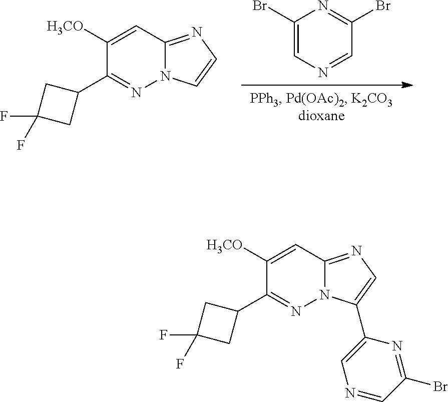

A mixture of 6-(3,3-difluorocyclobutyl)-7-methoxy-imidazo[1,2-b]pyridazine (0.700 g, 2.93 mmol), 2,6-dibromopyrazine (1.39 g, 5.85 mmol), triphenylphosphine (0.115 g, 0.439 mmol), 2,2-dimethylpropanoic acid (0.090 g, 0.878 mmol, 0.101 mL), potassium carbonate (1.21 g, 8.78 mmol), and palladium(II)acetate (0.066 g, 0.293 mmol) in dioxane (10 mL) was purged with nitrogen, and was then heated at 100° C. for 15 hours under nitrogen atmosphere. The reaction was then cooled to room temperature, diluted with water (20 mL), and extracted with ethyl acetate (3×20 mL). The organic extracts were combined, washed with saturated aqueous sodium chloride solution (20 mL), dried over anhydrous sodium sulfate, filtered, and concentrated under reduced pressure. The resulting product was purified by HPLC (Waters Xbridge BEH C18 column, 10 micron, 250×50 mm; 25-65% acetonitrile in water containing 0.1% sodium bicarbonate) to provide the title compound: LCMS m/z 396.1 [M+H]⁺.

### Step B. tert-butyl (3S,4S)-3-((6-(6-(3,3-difluorocyclobutyl)-7-methoxyimidazo[1,2-b]pyridazin-3-yl)pyrazin-2-yl)amino)-4-fluoropyrrolidine-1-carboxylate

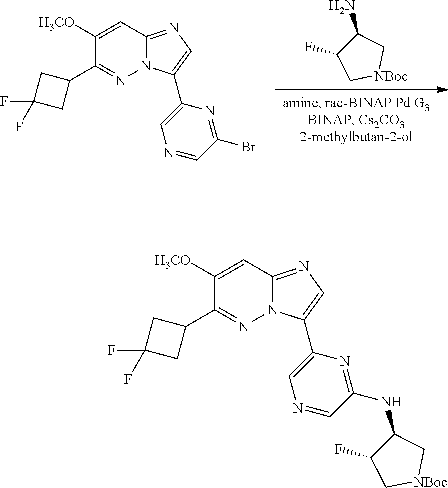

A mixture of 3-(6-bromopyrazin-2-yl)-6-(3,3-difluorocyclobutyl)-7-methoxy-imidazo[1,2-b]pyridazine (0.020 g, 0.050 mmol), tert-butyl (3S,4S)-3-amino-4-fluoro-pyrrolidine-1-carboxylate (0.015 g, 0.076 mmol), methanesulfonato[2,2-bis(diphenylphosphino)-1,1-binaphthyl](2-amino-1,1-biphenyl-2-yl)palladium(II) (0.005 g, 0.005 mmol), (R)-(+)-2,2′-bis(diphenylphosphino)-1,1′-binaphthyl (0.005 g, 0.008 mmol), and cesium carbonate (0.041 g, 0.126 mmol) in 2-methylbutan-2-ol (3 mL) was purged with nitrogen, and was then heated at 80° C. for 4 hours under nitrogen atmosphere. The reaction was cooled to room temperature, filtered, and concentrated under reduced pressure to provide the title compound: LCMS m/z 520.20 [M+H]⁻.

### Step C. 6-(6-(3,3-difluorocyclobutyl)-7-methoxyimidazo[1,2-b]pyridazin-3-yl)-N-((3S,4S)-4-fluoropyrrolidin-3-yl)pyrazin-2-amine

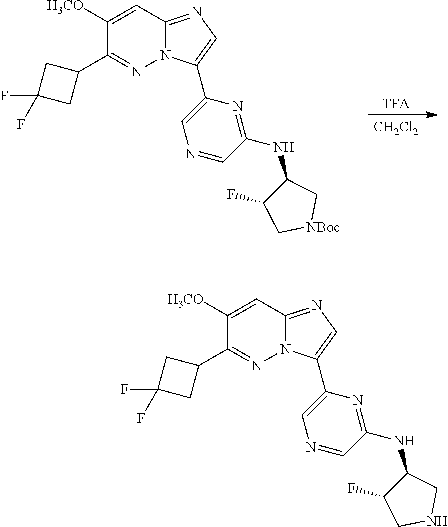 [SMILES: COc1cc2ncc(-c3cncc(N[C@H]4CN(C(=O)OC(C)(C)C)C[C@@H]4F)n3)n2nc1C1CC(F)(F)C1 ; "CH2Cl2" ; COc1cc2ncc(-c3cncc(N[C@H]4CNC[C@@H]4F)n3)n2nc1C1CC(F)(F)C1 ; "TFA"]

To a solution of tert-butyl (3S,4S)-3-[[6-[6-(3,3-difluorocyclobutyl)-7-methoxy-imidazo[1,2-b]pyridazin-3-yl]pyrazin-2-yl]amino]-4-fluoro-pyrrolidine-1-carboxylate (0.025 mg, 0.048 mmol) in dichloromethane (2 mL) was added trifluoroacetic acid (0.50 mL). The resulting reaction was stirred at room temperature for 1 hour, and was then concentrated under reduced pressure. The crude product thus obtained was purified by HPLC (Phenomenex Luna C18 column, 3 micron, 80×30 mm; 5-35% acetonitrile in water containing 0.1% trifluoroacetic acid) to provide the title compound: LCMS m/z 420.1 [M+H]⁺; ¹HNMR (400 MHz, CD₃OD) δ 9.05 (s, 1H), 8.37 (s, 1H), 8.00 (s, 1H), 7.57 (s, 1H), 5.56-5.40 (m, 1H), 4.83-4.78 (m, 1H), 4.09 (s, 3H), 3.96-3.85 (m, 2H), 3.80-3.66 (m, 2H), 3.64-3.56 (m, 1H), 3.14-3.03 (m, 4H).

The compounds in Table 16 were all prepared using the synthetic procedures described in Example 17.

TABLE 16
Additional compounds prepared according to Example 17.

| Compound | | | |
|---|---|---|---|
| # | Structure | IUPAC Name | LCMS |
| 72a | 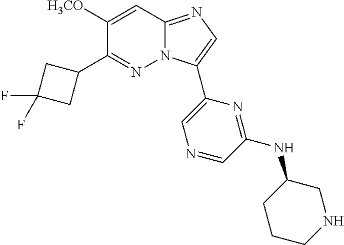 [SMILES: COc1cc2ncc(-c3cncc(N[C@@H]4CCCNC4)n3)n2nc1C1CC(F)(F)C1] | (R)-6-(6-(3,3- difluorocyclobutyl)-7- methoxyimidazo[1,2- b]pyridazin-3-yl)-N- (piperidin-3-yl)pyrazin- 2-amine | 415.19 |
| 73a | 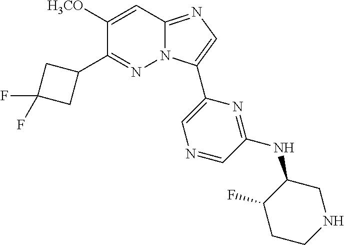 [SMILES: COc1cc2ncc(-c3cncc(N[C@H]4CNCC[C@@H]4F)n3)n2nc1C1CC(F)(F)C1] | 6-(6-(3,3- difluorocyclobutyl)-7- methoxyimidazo[1,2- b]pyridazin-3-yl)-N- ((3S,4S)-4- fluoropiperidin-3- yl)pyrazin-2-amine | 433.18 |

### Example 18

### Exemplary Synthetic Procedure #16 (Compounds 74a-91a)

### Compound 74a, N-((3S,4S)-4-fluoropiperidin-3-yl)-4-(7-methoxy-6-(spiro[3.3]heptan-2-yl)imidazo[1,2-b]pyridazin-3-yl)pyrimidin-2-amine

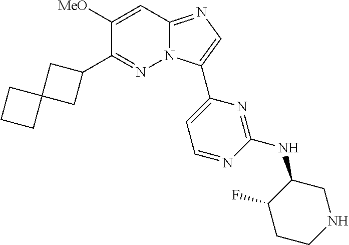 [SMILES: COc1cc2ncc(-c3ccnc(N[C@H]4CNCC[C@@H]4F)n3)n2nc1C1CC2(CCC2)C1]

### Step A. 6-chloro-4-methoxy-3-(spiro[3.3]heptan-2-yl)pyridazine

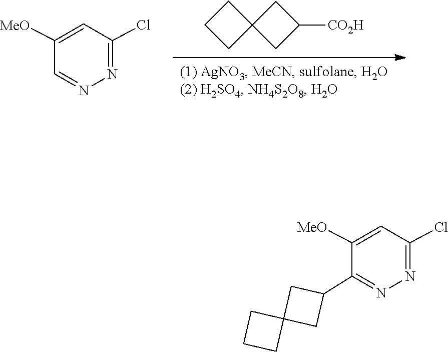 [SMILES: COc1cnnc(Cl)c1 ; "(1) AgNO3, MeCN, sulfolane, H2O(2) H2SO4, NH4S2O8, H2O" ; O=C(O)C1CC2(CCC2)C1 ; COc1cc(Cl)nnc1C1CC2(CCC2)C1]

To a solution of 3-chloro-5-methoxy-pyridazine (5.50 g, 38.1 mmol) in sulfolane (15 mL), water (75 mL), and acetonitrile (15 mL) were added spiro[3.3]heptane-2-carboxylic acid (8.00 g, 57.1 mmol) and silver nitrate (6.46 g, 38.1 mmol). The resulting reaction was heated to 50° C. A solution of sulfuric acid (10.78 g, 110.0 mmol, 5.86 mL) in water (30 mL) was added, followed by dropwise addition of a solution of ammonia sulfoxy hydrogen sulfate (14.33 g, 62.78 mmol, 13.64 mL) in water (30 mL) over 35 minutes. The reaction was heated to 70° C. for 20 minutes, and was then cooled to room temperature and stirred for 24 hours. The reaction was then neutralized to pH ~7 via addition of aqueous sodium hydroxide solution (30% w/w, 10 mL), diluted with water (300 mL), and extracted with ethyl acetate (3×200 mL). The organic extracts were combined, washed with saturated aqueous sodium chloride solution (3×200 mL), dried over anhydrous sodium sulfate, filtered, and concentrated under reduced pressure. The resulting crude product was purified by flash chromatography on silica gel (0-100% ethyl acetate in petroleum ether) to provide the title compound: LCMS m/z 238.9 [M+H]⁺; ¹H NMR (400 MHz, CD₃OD) δ 7.25 (s, 1H), 3.96 (s, 3H), 3.78-3.67 (q, 1H), 2.42-2.34 (m, 4H), 2.25-2.12 (m, 2H), 1.98-1.77 (m, 4H).

### Step B. tert-butyl (5-methoxy-6-(spiro[3.3]heptan-2-yl)pyridazin-3-yl)carbamate

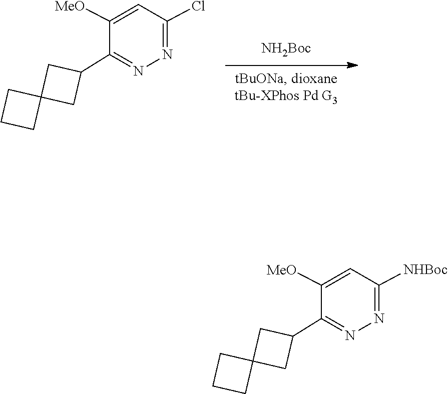 [SMILES: COc1cc(Cl)nnc1C1CC2(CCC2)C1 ; "tBuONa, dioxanetBu-XPhos Pd G3" ; COc1cc(NC(=O)OC(C)(C)C)nnc1C1CC2(CCC2)C1 ; "NH2Boc"]

A mixture of 6-chloro-4-methoxy-3-spiro[3.3]heptan-2-yl-pyridazine (5.50 g, 23.0 mmol), tert-butyl carbamate (8.10 g, 69.1 mmol), [2-(2-aminophenyl)phenyl]-methylsulfonyloxypalladium ditert-butyl-[2-(2,4,6-triisopropylphenyl)phenyl]phosphane (1.83 g, 2.30 mmol), and sodium tert-butoxide solution (2 M in THF, 23.04 mL) in dioxane (120 mL) was purged with nitrogen, and was then heated at 100° C. for 16 hours under nitrogen atmosphere. The reaction was then cooled to room temperature, diluted with water (300 mL), and extracted with ethyl acetate (2×250 mL). The organic extracts were combined, washed with saturated aqueous sodium chloride solution (300 mL), dried over sodium sulfate, filtered, and concentrated under reduced pressure. The resulting crude product was purified by flash chromatography on silica gel (0-100% ethyl acetate in petroleum ether) to provide the title compound: LCMS m/z 320.0 [M+H]⁺; ¹H NMR (400 MHz, CD₃OD) δ 7.71 (s, 1H), 6.97 (s, 1H), 3.93 (s, 3H), 3.67 (quin, J=8.8 Hz, 1H), 2.40-2.28 (m, 4H), 2.21-2.13 (m, 2H), 1.96-1.81 (m, 4H), 1.54 (s, 10H), 1.33-1.16 (m, 3H), 0.98-0.79 (m, 3H).

### Step C. 5-methoxy-6-(spiro[3.3]heptan-2-yl)pyridazin-3-amine

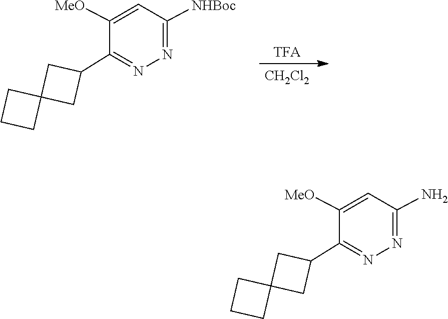 [SMILES: COc1cc(NC(=O)OC(C)(C)C)nnc1C1CC2(CCC2)C1 ; "CH2Cl2" ; COc1cc(N)nnc1C1CC2(CCC2)C1 ; "TFA"]

A solution of tert-butyl N-(5-methoxy-6-spiro[3.3]heptan-2-yl-pyridazin-3-yl)carbamate (1.50 g, 4.70 mmol) in trifluoroacetic acid (6 mL) was stirred at room temperature for 30 minutes. The reaction was then concentrated under reduced pressure to provide the title compound: LCMS m/z 220.0 [M+H]⁺.

### Step D. 7-methoxy-6-(spiro[3.3]heptan-2-yl)imidazo[1,2-b]pyridazine

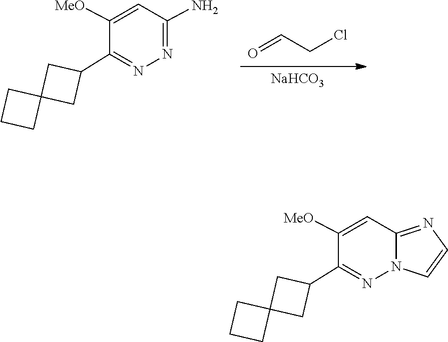

A mixture of 5-methoxy-6-spiro[3.3]heptan-2-yl-pyridazin-3-amine (1.50 g, 6.84 mmol), 2-chloroacetaldehyde (6.71 g, 34.2 mmol, 5.50 mL, 40% purity), and sodium bicarbonate (0.575 g, 6.84 mmol) in ethanol (10 mL) was purged with nitrogen, and was then heated at 80° C. for 4 hours under nitrogen atmosphere. The reaction was then cooled to room temperature, diluted with water (100 mL), and extracted with ethyl acetate (3×60 mL). The organic extracts were combined, washed with saturated aqueous sodium chloride solution (100 mL), dried over sodium sulfate, filtered, and concentrated under reduced pressure. The resulting crude product was purified by flash chromatography on silica gel (20-100% ethyl acetate in petroleum ether) to provide the title compound: LCMS m/z 244.1 [M+H]⁺; 1H NMR (400 MHz, CD₃OD) δ 7.85 (s, 1H), 7.47 (s, 1H), 7.15 (s, 1H), 3.92 (s, 3H), 3.72-3.65 (q, 1H), 2.39-2.29 (m, 4H), 2.20-2.15 (m, 2H), 1.94-1.82 (m, 4H).

### Step E. 3-iodo-7-methoxy-6-(spiro[3.3]heptan-2-yl)imidazo[1,2-b]pyridazine

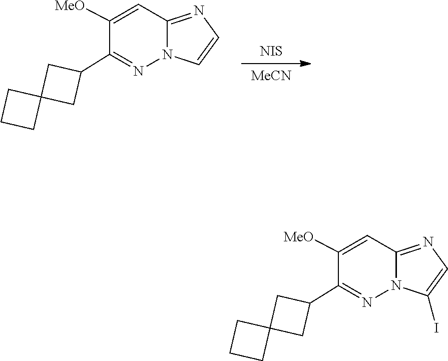

To a cooled 0° C. solution of 7-methoxy-6-spiro[3.3]heptan-2-yl-imidazo[1,2-b]pyridazine (0.750 g, 3.08 mmol) in acetonitrile (5 mL) was added 1-iodopyrrolidine-2,5-dione (0.694 g, 3.08 mmol). The resulting reaction was stirred for 4 hours while warming to room temperature. The reaction was then basified to pH ~9 by addition of aqueous sodium hydroxide solution (2 M, 4 mL), diluted with water (20 mL), and extracted with ethyl acetate (3×15 mL). The organic extracts were combined, washed with saturated aqueous sodium chloride solution (20 mL), dried over sodium sulfate, filtered, and concentrated under reduced pressure. The resulting crude product was purified by flash chromatography on silica gel (5-40% ethyl acetate in petroleum ether) to provide the title compound: LCMS m/z 369.9 [M+H]⁺; ¹H NMR (400 MHz, CD₃OD) δ 7.55 (s, 1H), 7.19 (s, 1H), 3.95 (s, 3H), 2.43-2.41 (m, 4H), 2.24-2.15 (m, 3H), 1.99-1.97 (m, 2H), 1.92-1.83 (m, 2H).

### Step F. 3-(2-chloropyrimidin-4-yl)-7-methoxy-6-(spiro[3.3]heptan-2-yl)imidazo[1,2-b]pyridazine

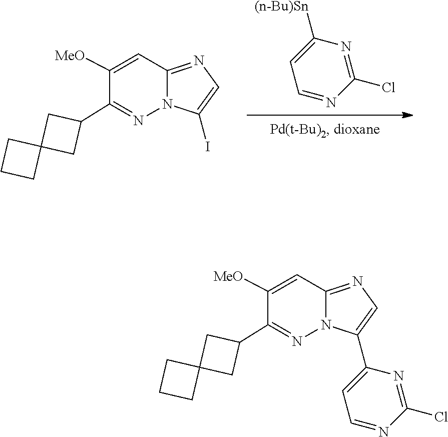

A mixture of 3-iodo-7-methoxy-6-spiro[3.3]heptan-2-yl-imidazo[1,2-b]pyridazine (0.100 g, 0.271 mmol), tributyl-(2-chloropyrimidin-4-yl)stannane (0.219 g, 0.542 mmol), di-(tert-butylphosphine)palladium (0.014 g, 0.027 mmol) in dioxane (4 mL) was purged with nitrogen, and was then heated at 100° C. for 16 hours under nitrogen atmosphere. The reaction was then cooled to room temperature, diluted with water (8 mL), and extracted with ethyl acetate (3×5 mL). The organic extracts were combined, washed with saturated aqueous sodium chloride solution (5 mL), dried over sodium sulfate, filtered, and concentrated under reduced pressure. The resulting crude product was purified by flash chromatography on silica gel (5-70% ethyl acetate in petroleum ether) to provide the title compound: LCMS m/z 355.95 [M+H]⁺.

### Step G. tert-butyl (3S,4S)-4-fluoro-3-((4-(7-methoxy-6-(spiro[3.3]heptan-2-yl)imidazo[1,2-b]pyridazin-3-yl)pyrimidin-2-yl)amino)piperidine-1-carboxylate

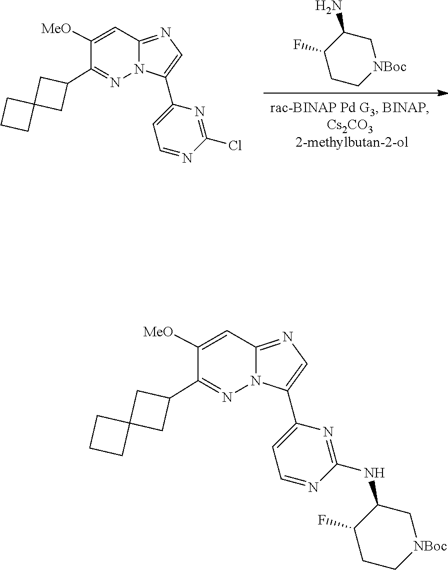

A mixture of 3-(2-chloropyrimidin-4-yl)-7-methoxy-6-spiro[3.3]heptan-2-yl-imidazo[1,2-b]pyridazine (0.020 g, 0.056 mmol), tert-butyl (3S,4S)-3-amino-4-fluoro-piperidine-1-carboxylate (0.018 g, 0.084 mmol), cesium carbonate (0.055 g, 0.168 mmol), methanesulfonato[2,2-bis(diphenylphosphino)-1,1-binaphthyl](2-amino-1,1-biphenyl-2-yl)palladium(II) (0.006 g, 0.006 mmol), and (R)-(+)-2,2′-bis(diphenylphosphino)-1,1′-binaphthyl (0.005 g, 0.008 mmol) in 2-methylbutan-2-ol (3 mL) was purged with nitrogen, and was then heated at 80° C. for 4 hours under nitrogen atmosphere. The reaction was then cooled to room temperature and concentrated under reduced pressure to provide the title compound: LCMS m/z 538.25 [M+H]⁻.

### Step H. N-((3S,4S)-4-fluoropiperidin-3-yl)-4-(7-methoxy-6-(spiro[3.3]heptan-2-yl)imidazo[1,2-b]pyridazin-3-yl)pyrimidin-2-amine

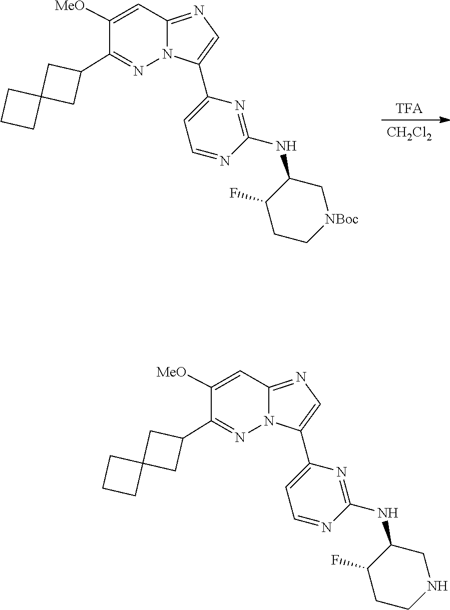 [SMILES: COc1cc2ncc(-c3ccnc(N[C@H]4CN(C(=O)OC(C)(C)C)CC[C@@H]4F)n3)n2nc1C1CC2(CCC2)C1 ; "CH2Cl2" ; "TFA" ; COc1cc2ncc(-c3ccnc(N[C@H]4CNCC[C@@H]4F)n3)n2nc1C1CC2(CCC2)C1]

To a solution of tert-butyl (3S,4S)-4-fluoro-3-[[4-(7-methoxy-6-spiro[3.3]heptan-2-yl-imidazo[1,2-b]pyridazin-3-yl)pyrimidin-2-yl]amino]piperidine-1-carboxylate (0.047 g, 0.087 mmol) in dichloromethane (3 mL) was added trifluoroacetic acid (1 mL). The resulting reaction was stirred at room temperature for 1 hour, and was then concentrated under reduced pressure. The crude product thus obtained was purified by HPLC (Waters Xbridge BEH C18 column, 3 micron, 100×25 mm; 20-80% o acetonitrile in water containing 0.100 ammonium bicarbonate) to provide the title compound: LCMS m/z 438.1 [M+H]⁺; ¹H NMR (400 MHz, CD₃OD) δ 8.38 (d, J=5.2 Hz, 1H), 7.92 (d, J=5.6 Hz, 1H), 7.31 (s, 1H), 4.75-4.53 (m, 1H), 4.19-4.18 (m, 1H), 3.98 (s, 3H), 3.82-3.77 (m, 1H), 3.40-3.32 (m, 2H), 3.16-3.03 (m, 1H), 2.75-2.61 (m, 1H), 2.57-2.35 (m, 5H), 2.25-2.12 (m, 3H), 2.01-1.73 (m, 5H).

The compound in Table 17 were all prepared using the synthetic procedures described in Example 18.

TABLE 17
Additional compounds prepared according to Example 18.

| Compound | | | |
|---|---|---|---|
| # | Structure | IUPAC Name | LCMS |
| 75a | 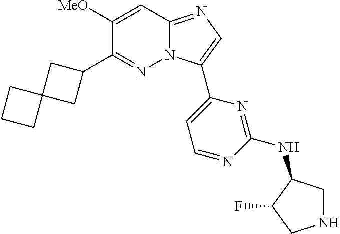 [SMILES: COc1cc2ncc(-c3ccnc(N[C@H]4CNC[C@@H]4F)n3)n2nc1C1CC2(CCC2)C1] | N-((3S,4S)-4- fluoropyrrolidin-3-yl)-4- (7-methoxy-6- (spiro[3.3]heptan-2- yl)imidazo[1,2- b]pyridazin-3- yl)pyrimidin-2-amine | 424.1 |
| 76a | 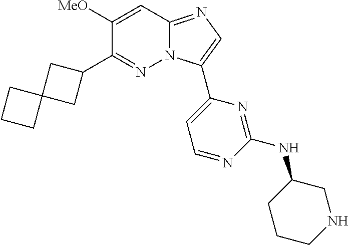 [SMILES: COc1cc2ncc(-c3ccnc(N[C@@H]4CCCNC4)n3)n2nc1C1CC2(CCC2)C1] | (R)-4-(7-methoxy-6- (spiro[3.3]heptan-2- yl)imidazo[1,2- b]pyridazin-3-yl)-N- (piperidin-3- yl)pyrimidin-2-amine | 420.1 |
| 77a | 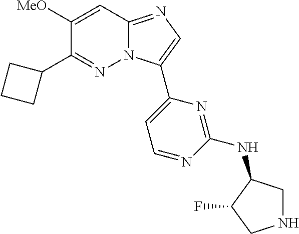 [SMILES: COc1cc2ncc(-c3ccnc(N[C@H]4CNC[C@@H]4F)n3)n2nc1C1CCC1] | 4-(6-cyclobutyl-7- methoxyimidazo[1,2- b]pyridazin-3-yl)-N- ((3S,4S)-4- fluoropyrrolidin-3- yl)pyrimidin-2-amine | 383.2 |
| 78a | 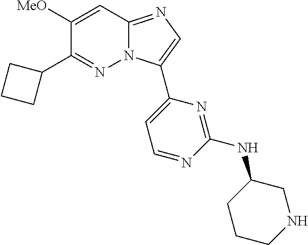 [SMILES: COc1cc2ncc(-c3ccnc(N[C@@H]4CCCNC4)n3)n2nc1C1CCC1] | (R)-4-(6-cyclobutyl-7- methoxyimidazo[1,2- b]pyridazin-3-yl)-N- (piperidin-3- yl)pyrimidin-2-amine | 380.1 |
| 79a | 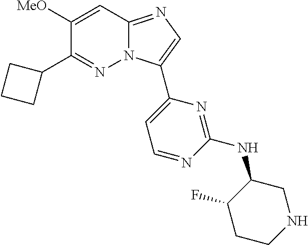 [SMILES: COc1cc2ncc(-c3ccnc(N[C@H]4CNCC[C@@H]4F)n3)n2nc1C1CCC1] | 4-(6-cyclobutyl-7- methoxyimidazo[1,2- b]pyridazin-3-yl)-N- ((3S,4S)-4- fluoropiperidin-3- yl)pyrimidin-2-amine | 397.2 |
| 80a | 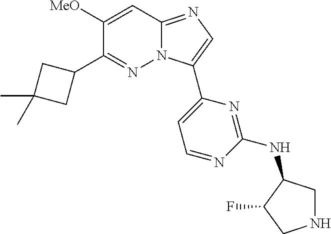 [SMILES: COc1cc2ncc(-c3ccnc(N[C@H]4CNC[C@@H]4F)n3)n2nc1C1CC(C)(C)C1] | 4-(6-(3,3- dimethylcyclobutyl)-7- methoxyimidazo[1,2- b]pyridazin-3-yl)-N- ((3S,4S)-4- fluoropyrrolidin-3- yl)pyrimidin-2-amine | 412.1 |
| 81a | 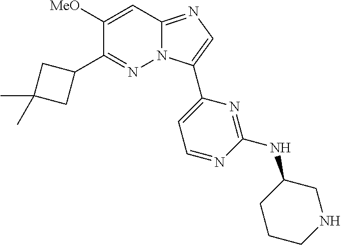 [SMILES: COc1cc2ncc(-c3ccnc(N[C@@H]4CCCNC4)n3)n2nc1C1CC(C)(C)C1] | 3-(2-chloropyrimidin-4- yl)-6-(3,3- dimethylcyclobutyl)-7- methoxyimidazo[1,2- b]pyridazine | 408.1 |
| 82a | 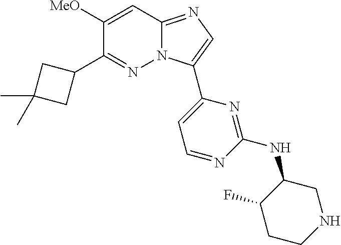 [SMILES: COc1cc2ncc(-c3ccnc(N[C@H]4CNCC[C@@H]4F)n3)n2nc1C1CC(C)(C)C1] | 4-(6-(3,3- dimethylcyclobutyl)-7- methoxyimidazo[1,2- b]pyridazin-3-yl)-N- ((3S,4S)-4- fluoropiperidin-3- yl)pyrimidin-2-amine | 426.1 |
| 83a | 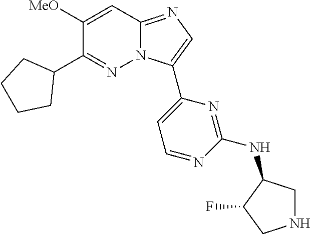 [SMILES: COc1cc2ncc(-c3ccnc(N[C@H]4CNC[C@@H]4F)n3)n2nc1C1CCCC1] | 4-(6-cyclopentyl-7- methoxyimidazo[1,2- b]pyridazin-3-yl)-N- ((3S,4S)-4- fluoropyrrolidin-3- yl)pyrimidin-2-amine | 398.1 |
| 84a | 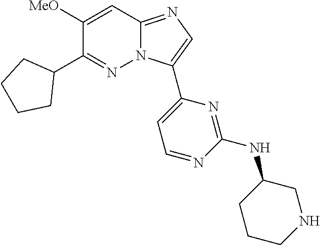 [SMILES: COc1cc2ncc(-c3ccnc(N[C@@H]4CCCNC4)n3)n2nc1C1CCCC1] | (R)-4-(6-cyclopentyl-7- methoxyimidazo[1,2- b]pyridazin-3-yl)-N- (piperidin-3- yl)pyrimidin-2-amine | 394.0 |
| 85a | 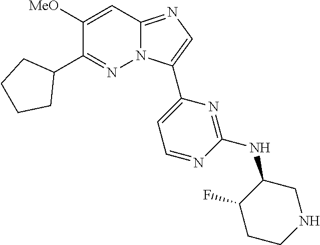 [SMILES: COc1cc2ncc(-c3ccnc(N[C@H]4CNCC[C@@H]4F)n3)n2nc1C1CCCC1] | 4-(6-cyclopentyl-7- methoxyimidazo[1,2- b]pyridazin-3-yl)-N- ((3S,4S)-4- fluoropiperidin-3- yl)pyrimidin-2-amine | 412.1 |
| 86a | 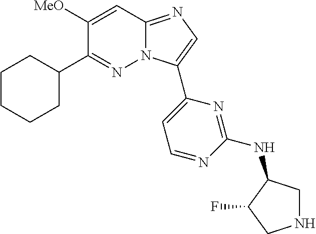 [SMILES: COc1cc2ncc(-c3ccnc(N[C@H]4CNC[C@@H]4F)n3)n2nc1C1CCCCC1] | 4-(6-cyclohexyl-7- methoxyimidazo[1,2- b]pyridazin-3-yl)-N- ((3S,4S)-4- fluoropyrrolidin-3- yl)pyrimidin-2-amine | 412.1 |
| 87a | 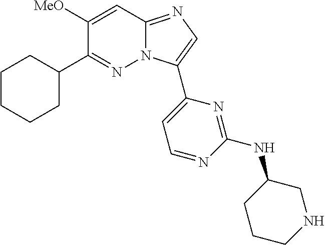 [SMILES: COc1cc2ncc(-c3ccnc(N[C@@H]4CCCNC4)n3)n2nc1C1CCCCC1] | (R)-4-(6-cyclohexyl-7- methoxyimidazo[1,2- b]pyridazin-3-yl)-N- (piperidin-3- yl)pyrimidin-2-amine | 408.1 |
| 88a | 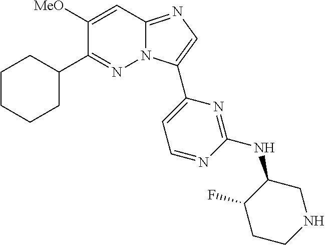 [SMILES: COc1cc2ncc(-c3ccnc(N[C@H]4CNCC[C@@H]4F)n3)n2nc1C1CCCCC1] | 4-(6-cyclohexyl-7- methoxyimidazo[1,2- b]pyridazin-3-yl)-N- ((3S,4S)-4- fluoropiperidin-3- yl)pyrimidin-2-amine | 426.10 |
| 89a | 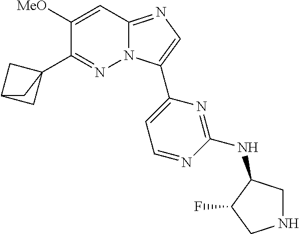 [SMILES: COc1cc2ncc(-c3ccnc(N[C@H]4CNC[C@@H]4F)n3)n2nc1C12CC(C1)C2] | 4-(6- (bicyclo[1.1.1]pentan-1- yl)-7- methoxyimidazo[1,2- b]pyridazin-3-yl)-N- ((3S,4S)-4- fluoropyrrolidin-3- yl)pyrimidin-2-amine | 396.1 |
| 90a | 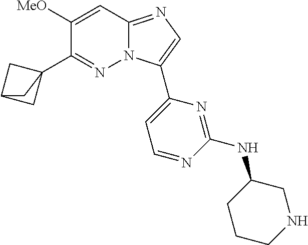 [SMILES: COc1cc2ncc(-c3ccnc(N[C@@H]4CCCNC4)n3)n2nc1C12CC(C1)C2] | (R)-4-(6- (bicyclo[1.1.1]pentan-1- yl)-7- methoxyimidazo[1,2- b]pyridazin-3-yl)-N- (piperidin-3- yl)pyrimidin-2-amine | 392.3 |
| 91a | 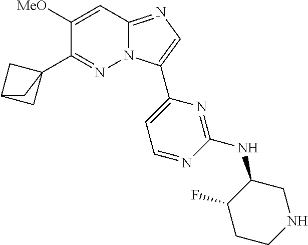 [SMILES: COc1cc2ncc(-c3ccnc(N[C@H]4CNCC[C@@H]4F)n3)n2nc1C12CC(C1)C2] | 4-(6- (bicyclo[1.1.1]pentan-1- yl)-7- methoxyimidazo[1,2- b]pyridazin-3-yl)-N- ((3S,4S)-4- fluoropiperidin-3- yl)pyrimidin-2-amine | 410.1 |

### Example 19

### Exemplary Synthetic Procedure #17 (Compounds 92a-94a)

### Compound 92a, 5-fluoro-N-((3S,4S)-4-fluoropiperidin-3-yl)-4-(7-methoxy-6-(oxetan-3-yl)imidazo[1,2-b]pyridazin-3-yl)pyrimidin-2-amine

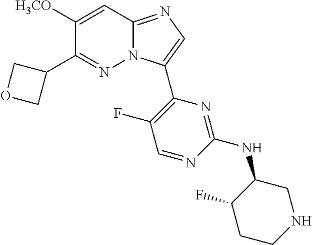 [SMILES: COc1cc2ncc(-c3nc(N[C@H]4CNCC[C@@H]4F)ncc3F)n2nc1C1COC1]

### Step A. 6-chloro-4-methoxy-3-(oxetan-3-yl)pyridazine

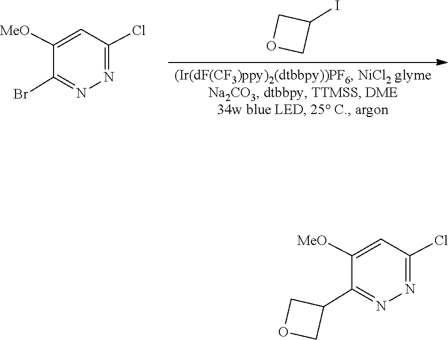

A mixture of 3-bromo-6-chloro-4-methoxy-pyridazine (1.00 g, 4.48 mmol), 3-iodooxetane (2.47 g, 13.4 mmol), bis[3,5-difluoro-2-[5-(trifluoromethyl)-2-pyridyl]phenyl]iridium(1+) 4-tert-butyl-2-(4-tert-butyl-2-pyridyl)pyridinehexafluorophosphate (0.100 g, 0.090 mmol), dichloronickel 1,2-dimethoxyethane (0.010 g, 0.045 mmol), sodium carbonate (0.949 g, 0.009 mmol), 4-tert-butyl-2-(4-tert-butyl-2-pyridyl)pyridine (0.012 g, 0.045 mmol), and bis(trimethylsilyl)silyl-trimethylsilane (1.11 g, 4.48 mmol, 1.38 mL) in 1,2-dimethoxyethane (15 mL) was purged with argon, and was then stirred and irradiated with two 34 W blue LEDs (vials approximately 6 cm away from the light source) at room temperature for 16 hours. The reaction was then diluted with water (15 mL) and extracted with ethyl acetate (3×15 mL). The organic extracts were combined, washed with saturated aqueous sodium chloride solution (15 mL), dried over anhydrous sodium sulfate, filtered, and concentrated under reduced pressure. The resulting crude product was purified by flash chromatography on silica gel (0-100% ethyl acetate in petroleum ether) to provide the title compound: LCMS m/z 201 [M+H]⁺; ¹H NMR (400 MHz, CD₃OD) δ 7.37 (s, 1H), 5.04-5.01 (m, 4H), 4.69-4.65 (m, 1H), 3.97 (s, 3H).

### Step B. tert-butyl (5-methoxy-6-(oxetan-3-yl)pyridazin-3-yl)carbamate

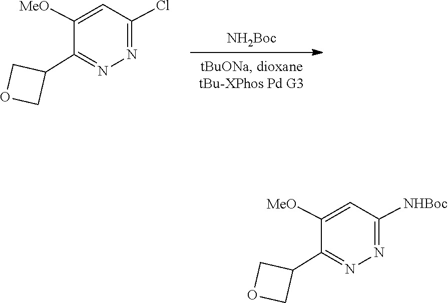 [SMILES: COc1cc(Cl)nnc1C1COC1 ; "tBuONa, dioxanetBu-XPhos Pd G3" ; COc1cc(NC(=O)OC(C)(C)C)nnc1C1COC1 ; "NH2Boc"]

A mixture of 6-chloro-4-methoxy-3-(oxetan-3-yl)pyridazine (4.00 g, 19.9 mmol), tert-butyl carbamate (7.01 g, 59.8 mmol), sodium tert-butoxide solution (2 M in THF, 19.90 mL), and [2-(2-aminophenyl)phenyl]-methylsulfonyloxypalladium ditert-butyl-[2-(2,4,6-triisopropylphenyl)phenyl]phosphane (1.58 g, 1.99 mmol) in dioxane (40 mL) was purged with nitrogen, and was then heated at 100° C. for 16 hours under nitrogen atmosphere. The reaction was then cooled to room temperature, diluted with water (20 mL), and extracted with ethyl acetate (3×30 mL). The organic extracts were combined, washed with saturated aqueous sodium chloride solution (3×20 mL), dried over anhydrous sodium sulfate, filtered, and concentrated under reduced pressure. The resulting crude product was purified by flash chromatography on silica gel (0-100% ethyl acetate in petroleum ether) to provide the title compound: LCMS m/z 282 [M+H]⁺; ¹H NMR (400 MHz, CD₃OD) δ 7.78 (s, 1H), 5.12-4.93 (m, 4H), 4.67-4.56 (m, 1H), 3.93 (s, 3H), 1.54 (s, 9H).

### Step C. 5-methoxy-6-(oxetan-3-yl)pyridazin-3-amine

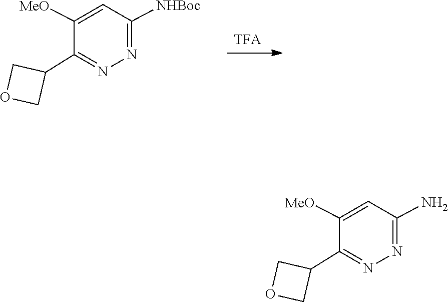 [SMILES: COc1cc(NC(=O)OC(C)(C)C)nnc1C1COC1 ; "TFA" ; COc1cc(N)nnc1C1COC1]

To a cooled 0° C. solution of tert-butyl N-[5-methoxy-6-(oxetan-3-yl) pyridazin-3-yl]carbamate (3.30 g, 11.7 mmol) in dichloromethane (50 mL) was added trifluoroacetic acid (25 mL). The resulting reaction was stirred at 0° C. for 15 minutes, and was then concentrated under reduced pressure. The crude product thus obtained was diluted with ethanol (10 mL), cooled to 0° C. and basified to pH ~9 by addition of sodium bicarbonate. The resulting mixture was filtered and concentrated under reduced pressure to provide the title compound: LCMS m/z 182.1 [M+H]⁺.

### Step D. 3-(2-bromo-5-fluoropyrimidin-4-yl)-7-methoxy-6-(oxetan-3-yl)imidazo[1,2-b]pyridazine

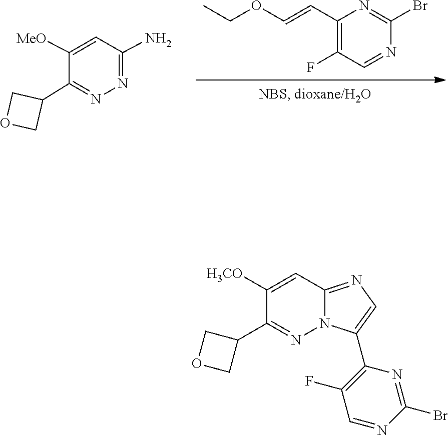

To a cooled 0° C. solution of 2-bromo-4-[(E)-2-ethoxyvinyl]-5-fluoro-pyrimidine (0.818 g, 3.31 mmol) in dioxane (3 mL) and water (1 mL) was added 1-bromopyrrolidine-2,5-dione (0.589 g, 3.31 mmol). The resulting reaction was stirred for 30 minutes while warming to room temperature. 5-Methoxy-6-(oxetan-3-yl) pyridazin-3-amine (1.00 g, 5.52 mmol) and sodium bicarbonate (0.927 g, 11.04 mmol) were then added, and the reaction was then heated to 80° C. for 15 hours. The reaction was then cooled to room temperature, basified to pH ~9 by addition of aqueous sodium hydroxide solution (2 M, 10 mL), and extracted with ethyl acetate (3×10 mL). The organic extracts were combined, washed with saturated aqueous sodium chloride solution (3×5 mL), dried over sodium sulfate, filtered, and concentrated under reduced pressure. The resulting product was purified by flash chromatography on silica gel (0-100% ethyl acetate in petroleum ether) to provide the title compound: LCMS m/z 382 [M+H]⁺; ¹H NMR (400 MHz, DMSO-d₆) δ 8.84 (d, J=3.3 Hz, 1H), 8.34 (d, J=3.0 Hz, 1H), 7.72 (s, 1H), 5.03-4.91 (m, 4H), 4.60 (m, 1H), 3.95 (s, 3H).

### Step E. tert-butyl (3S,4S)-4-fluoro-3-((5-fluoro-4-(7-methoxy-6-(oxetan-3-yl)imidazo[1,2-b]pyridazin-3-yl)pyrimidin-2-yl)amino)piperidine-1-carboxylate

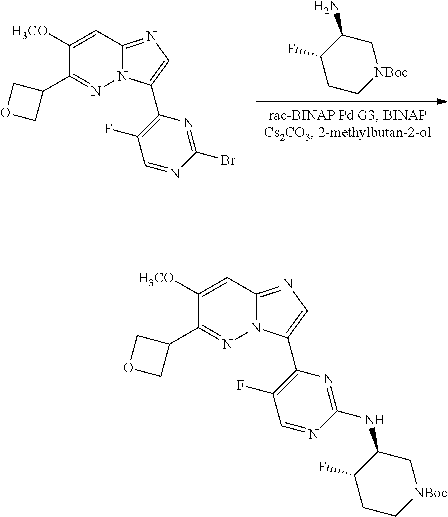

A mixture of 3-(2-bromo-5-fluoro-pyrimidin-4-yl)-7-methoxy-6-(oxetan-3-yl)imidazo[1,2-b]pyridazine (0.050 g, 0.132 mmol), tert-butyl (3S,4S)-3-amino-4-fluoro-piperidine-1-carboxylate (0.034 g, 0.158 mmol), [2-(2-aminophenyl)phenyl]-methylsulfonyloxypalladium[i-(2-diphenylphosphanyl-1-naphthyl)-2-naphthyl]-diphenylphosphane (0.013 g, 0.013 mmol), (R)-(+)-2,2′-bis(diphenylphosphino)-1,1′-binaphthyl (0.012 g, 0.020 mmol), and cesium carbonate (0.107 g, 0.329 mmol) in 2-methylbutan-2-ol (2 mL) was purged with nitrogen, and was then heated at 80° C. for 4 hours under nitrogen atmosphere. The reaction was then cooled to room temperature, filtered, and concentrated under reduced pressure to provide the title compound: LCMS m/z 518.4 [M+H]⁺.

### Step F. 5-fluoro-N-((3S,4S)-4-fluoropiperidin-3-yl)-4-(7-methoxy-6-(oxetan-3-yl)imidazo[1,2-b]pyridazin-3-yl)pyrimidin-2-amine

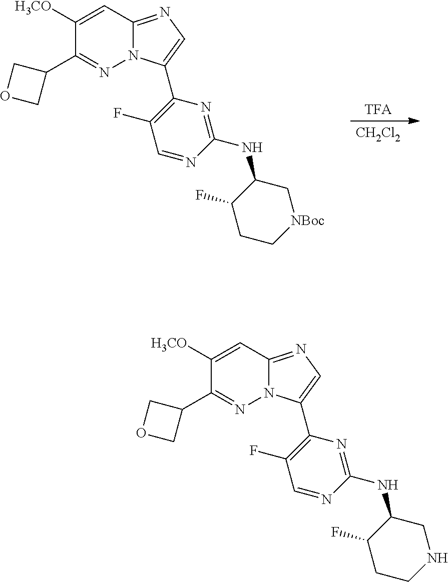 [SMILES: COc1cc2ncc(-c3nc(N[C@H]4CN(C(=O)OC(C)(C)C)CC[C@@H]4F)ncc3F)n2nc1C1COC1 ; "CH2Cl2" ; "TFA" ; COc1cc2ncc(-c3nc(N[C@H]4CNCC[C@@H]4F)ncc3F)n2nc1C1COC1]

To a solution of tert-butyl (3S,4S)-4-fluoro-3-[[5-fluoro-4-[7-methoxy-6-(oxetan-3-yl)imidazo[1,2-b]pyridazin-3-yl]pyrimidin-2-yl]amino]piperidine-1-carboxylate (0.080 g, 0.155 mmol) in dichloromethane (1 mL) was added trifluoroacetic acid (0.50 mL). The resulting reaction was stirred at room temperature for 15 minutes, and was then concentrated under reduced pressure. The crude product thus obtained was purified by HPLC (Phenomenex Luna C18 column, 3 micron, 75×30 mm; 10-40% acetonitrile in water containing 0.1% trifluoroacetic acid) to provide the title compound: LCMS m/z 418.0 [M+H]⁺; ¹H NMR (400 MHz, CD₃OD) δ 8.32 (d, J=2.9 Hz, 1H), 8.07 (d, J=2.1 Hz, 1H), 7.40 (s, 1H), 5.12-5.02 (m, 4H), 4.68 (br t, J=7.8 Hz, 2H), 4.24-4.14 (m, 1H), 3.99 (s, 3H), 3.30-3.25 (m, 1H), 3.09-2.98 (m, 1H), 2.64-2.60 (m, 1H), 2.55-2.46 (m, 1H), 2.15-2.13 (m, 1H), 1.79-1.65 (m, 1H).

The compounds in Table 18 were all prepared using the synthetic procedures described in Example 19.

TABLE 18
Additional compounds prepared according to Example 19.

| Compound | | | |
|---|---|---|---|
| # | Structure | IUPAC Name | LCMS |
| 93a | 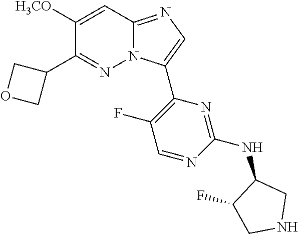 [SMILES: COc1cc2ncc(-c3nc(N[C@H]4CNC[C@@H]4F)ncc3F)n2nc1C1COC1] | 5-fluoro-N-((3S,4S)-4- fluoropyrrolidin-3-yl)-4- (7-methoxy-6-(oxetan-3- yl)imidazo[1,2- b]pyridazin-3- yl)pyrimidin-2-amine | 404.1 |
| 94a | 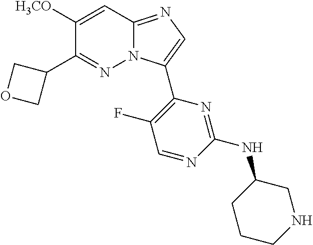 [SMILES: COc1cc2ncc(-c3nc(N[C@@H]4CCCNC4)ncc3F)n2nc1C1COC1] | (R)-5-fluoro-4-(7- methoxy-6-(oxetan-3- yl)imidazo[1,2- b]pyridazin-3-yl)-N- (piperidin-3-yl)pyrimidin- 2-amine | 400.1 |

### Example 20

### Exemplary Synthetic Procedure #18 (Compounds 95a-97a)

### Compound 95a, N-((3S,4S)-4-fluoropyrrolidin-3-yl)-4-(7-methoxy-6-(oxetan-3-yl)imidazo[1,2-b]pyridazin-3-yl)pyrimidin-2-amine

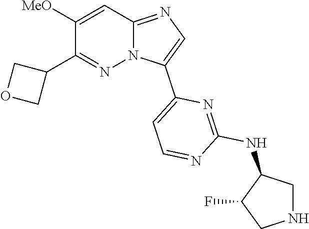 [SMILES: COc1cc2ncc(-c3ccnc(N[C@H]4CNC[C@@H]4F)n3)n2nc1C1COC1]

### Step A. 7-methoxy-6-(oxetan-3-yl)imidazo[1,2-b]pyridazine

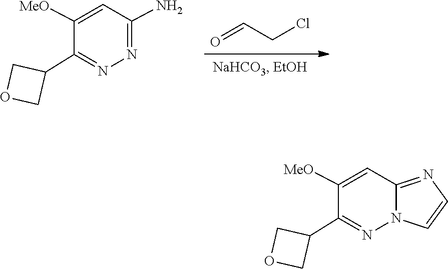

A mixture of 5-methoxy-6-(oxetan-3-yl)pyridazin-3-amine (3.00 g, 16.6 mmol), 2-chloroacetaldehyde (16.25 g, 82.79 mmol, 13.32 mL, 40% purity), and sodium bicarbonate (2.78 g, 33.1 mmol) in ethanol (20 mL) was purged with nitrogen, and was then heated at 80° C. for 4 hours under nitrogen atmosphere. The reaction was then cooled to room temperature, concentrated under reduced pressure, diluted with water (50 mL), and extracted with ethyl acetate (3×50 mL). The organic extracts were combined, washed with saturated aqueous sodium chloride solution (50 mL), dried over anhydrous sodium sulfate, filtered, and concentrated under reduced pressure. The resulting product was purified by flash chromatography on silica gel (0-100% ethyl acetate in petroleum ether) to provide the title compound: LCMS m/z 206 [M+H]⁺; ¹H NMR (400 MHz, DMSO-d₆) δ 8.09 (s, 1H), 7.54 (d, J=1.0 Hz, 1H), 7.50 (s, 1H), 4.91-4.82 (m, 4H), 4.61-4.52 (m, 1H), 3.87 (s, 3H).

### Step B. 3-iodo-7-methoxy-6-(oxetan-3-yl)imidazo[1,2-b]pyridazine

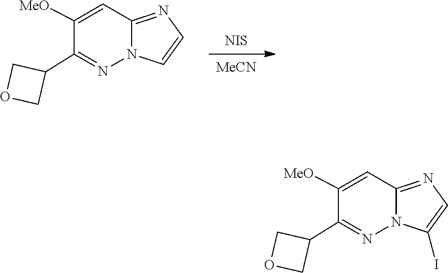

To a cooled 0° C. solution of 7-methoxy-6-(oxetan-3-yl)imidazo[1,2-b]pyridazine (0.900 g, 4.39 mmol) in acetonitrile (10 mL) was added 1-iodopyrrolidine-2,5-dione (1.09 g, 4.82 mmol). The resulting reaction was stirred for 1 hour while warming to room temperature, and was then filtered and concentrated under reduced pressure to provide the title compound: LCMS m/z 331.9 [M+H]⁺; ¹H NMR (400 MHz, CD₃OD) δ 7.60 (s, 1H), 7.29 (s, 1H), 5.12-5.02 (m, 4H), 4.68 (m, 1H), 3.95 (s, 3H).

### Step C. 3-(2-chloropyrimidin-4-yl)-7-methoxy-6-(oxetan-3-yl)imidazo[1,2-b]pyridazine

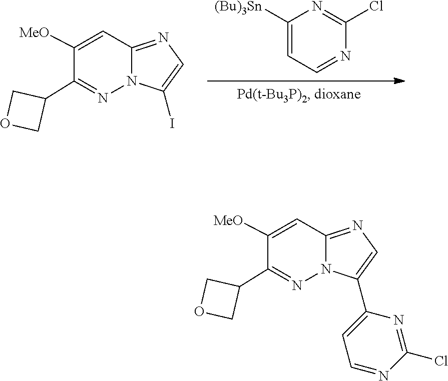

A mixture of 3-iodo-7-methoxy-6-(oxetan-3-yl)imidazo[1,2-b]pyridazine (0.130 g, 0.393 mmol), tributyl-(2-chloropyrimidin-4-yl)stannane (0.238 g, 0.589 mmol), and bis(tri-tert-butylphosphine)palladium (0.020 g, 0.039 mmol) in dioxane (3 mL) was purged with nitrogen, and was then heated at 100° C. for 16 hours under nitrogen atmosphere. The reaction was then cooled to room temperature, diluted with water (10 mL), and extracted with ethyl acetate (3×10 mL). The organic extracts were combined, washed with saturated aqueous sodium chloride solution (3×10 mL), dried over anhydrous sodium sulfate, filtered, and concentrated under reduced pressure. The resulting crude product was purified by flash chromatography on silica gel (0-100% ethyl acetate in petroleum ether) to provide the title compound: LCMS m/z 318.1 [M+H]⁺; ¹H NMR (400 MHz, DMSO-d₆) δ 8.84 (d, J=5.4 Hz, 1H), 8.52-8.43 (m, 2H), 7.75 (s, 1H), 5.01-4.95 (m, 4H), 4.73-4.63 (m, 1H), 3.95 (s, 3H).

### Step C. tert-butyl (3S,4S)-3-fluoro-4-((4-(7-methoxy-6-(oxetan-3-yl)imidazo[1,2-b]pyridazin-3-yl)pyrimidin-2-yl)amino)pyrrolidine-1-carboxylate

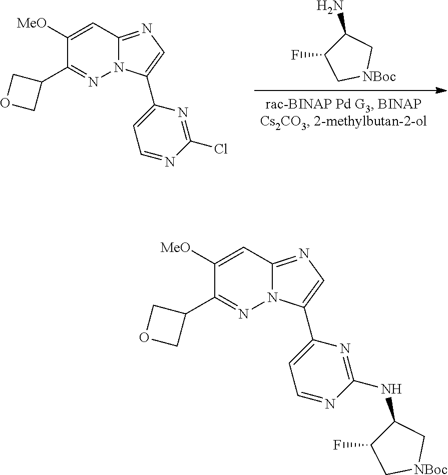

A mixture of 3-(2-chloropyrimidin-4-yl)-7-methoxy-6-(oxetan-3-yl)imidazo[1,2-b]pyridazine (0.040 g, 0.126 mmol), tert-butyl (3S,4S)-3-amino-4-fluoro-pyrrolidine-1-carboxylate (0.039 g, 0.189 mol), [2-(2-aminophenyl)phenyl]-methylsulfonyloxy-palladium[1-(2-diphenylphosphanyl-1-naphthyl)-2-naphthyl]-diphenyl-phosphane (0.012 g, 0.012 mmol), (R)-(+)-2,2′-bis(diphenylphosphino)-1,1′-binaphthyl (0.012 g, 0.019 mmol), and cesium carbonate (0.103 g, 0.315 mmol) in 2-methylbutan-2-ol (2 mL) was purged with nitrogen, and was then heated at 80° C. for 4 hours under nitrogen atmosphere. The reaction was then cooled to room temperature, filtered, and concentrated under reduced pressure to provide the title compound: LCMS m/z 486.2 [M+H]⁺.

### Step D. N-((3S,4S)-4-fluoropyrrolidin-3-yl)-4-(7-methoxy-6-(oxetan-3-yl)imidazo[1,2-b]pyridazin-3-yl)pyrimidin-2-amine

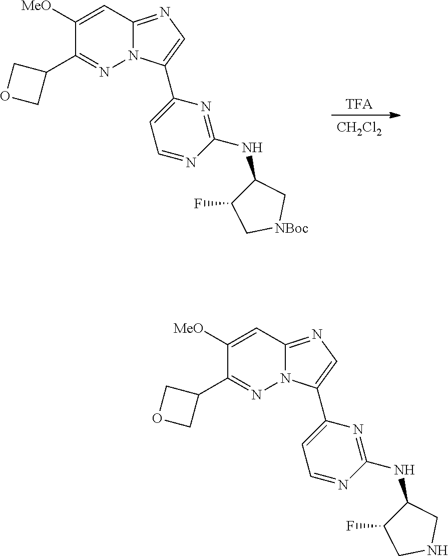 [SMILES: COc1cc2ncc(-c3ccnc(N[C@H]4CN(C(=O)OC(C)(C)C)C[C@@H]4F)n3)n2nc1C1COC1 ; "CH2Cl2" ; "TFA" ; COc1cc2ncc(-c3ccnc(N[C@H]4CNC[C@@H]4F)n3)n2nc1C1COC1]

To a solution of tert-butyl (3S,4S)-3-fluoro-4-[[4-[7-methoxy-6-(oxetan-3-yl)imidazo[1,2-b]pyridazin-3-yl]pyrimidin-2-yl]amino]pyrrolidine-1-carboxylate (0.080 g, 0.165 mmol) in dichloromethane (2 mL) was added trifluoroacetic acid (0.50 mL). The resulting reaction was stirred at room temperature for 15 minutes, and was then concentrated under reduced pressure. The crude product thus obtained was purified by HPLC (Waters Xbridge BEH C18 column, 10 micron, 100×30 mm; 1-40% acetonitrile in water containing 0.1% sodium bicarbonate) to provide the title compound: LCMS m/z 386.0 [M+H]⁺; ¹H NMR (400 MHz, DMSO-d₆) δ 8.46 (br d, J=5.1 Hz, 1H), 8.31 (br s, 1H), 7.75 (br d, J=5.1 Hz, 1H), 7.68 (s, 1H), 7.24 (br d, J=6.6 Hz, 1H), 5.14-4.90 (m, 5H), 4.67-4.63 (m, 1H), 4.41-4.28 (m, 1H), 3.93 (s, 3H), 3.25-3.23 (m, 1H), 3.15-2.89 (m, 2H), 2.79-2.71 (m, 1H).

The compounds in Table 19 were all prepared using the synthetic procedures described in Example 20.

TABLE 19
Additional compounds prepared according to Example 20.

| Compound | | | |
|---|---|---|---|
| # | Structure | IUPAC Name | LCMS |
| 96a | 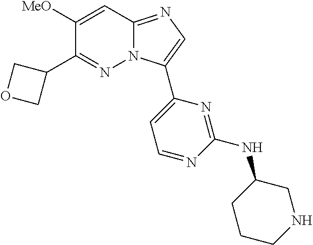 [SMILES: COc1cc2ncc(-c3ccnc(N[C@@H]4CCCNC4)n3)n2nc1C1COC1] | (R)-4-(7-methoxy-6- (oxetan-3-yl)imidazo[1,2- b]pyridazin-3-yl)-N- (piperidin-3-yl)pyrimidin- 2-amine | 382.1 |
| 97a | 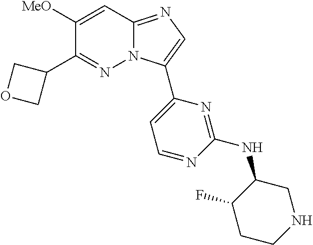 [SMILES: COc1cc2ncc(-c3ccnc(N[C@H]4CNCC[C@@H]4F)n3)n2nc1C1COC1] | N-((3S,4S)-4- fluoropiperidin-3-yl)-4-(7- methoxy-6-(oxetan-3- yl)imidazo[1,2- b]pyridazin-3- yl)pyrimidin-2-amine | 400.0 |

### Example 21

### Exemplary Synthetic Procedure #19 (Compounds 98a-100a)

### Compound 98a, 2-(3-(5-fluoro-2-(((3S,4S)-4-fluoropyrrolidin-3-yl)amino)pyrimidin-4-yl)imidazo[1,2-a]pyrazin-6-yl)propan-2-ol

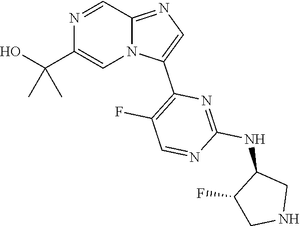 [SMILES: CC(C)(O)c1cn2c(-c3nc(N[C@H]4CNC[C@@H]4F)ncc3F)cnc2cn1]

### Step A. 5-(prop-1-en-2-yl)pyrazin-2-amine

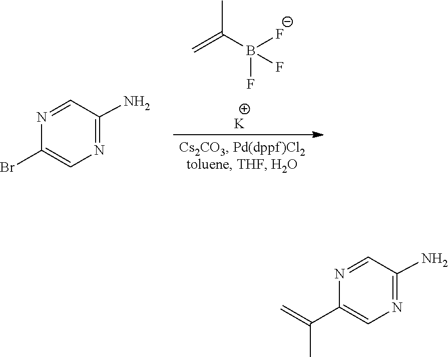

A mixture of 5-bromopyrazin-2-amine (2.00 g, 11.5 mmol), potassium isopropenyltrifluoroborate (1.70 g, 11.5 mmol), [1,1-bis(diphenylphosphino)ferrocene]dichloropalladium(II) (0.841 g, 1.15 mmol), and cesium carbonate (11.24 g, 34.48 mmol) in toluene (90 mL), tetrahydrofuran (30 mL), and water (30 mL) was purged with nitrogen, and was then heated at 100° C. for 16 hours under nitrogen atmosphere. The reaction was then cooled to room temperature, diluted with water (150 mL), and extracted with ethyl acetate (3×150 mL). The organic extracts were combined, washed with saturated aqueous sodium chloride solution (150 mL), dried over sodium sulfate, filtered, and concentrated under reduced pressure. The resulting crude product was purified by flash chromatography on silica gel (0-60% ethyl acetate in petroleum ether) to provide the title compound: LCMS m/z 136.1 [M+H]⁺; ¹H NMR (400 MHz, CD₃OD) δ 8.07 (d, J=1.2 Hz, 1H), 7.91 (d, J=1.6 Hz, 1H), 5.64 (s, 1H), 5.08 (s, 1H), 2.12 (s, 3H).

### Step B. 2-(5-aminopyrazin-2-yl)propan-2-ol

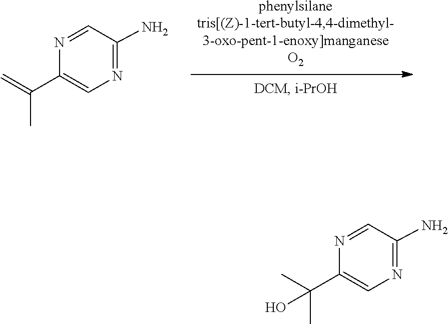

To a solution of 5-isopropenylpyrazin-2-amine (0.800 g, 5.92 mmol) in propan-2-ol (24 mL) was added phenylsilane (2.24 g, 20.72 mmol, 2.56 mL). The resulting reaction was purged with oxygen and cooled to 0° C. A solution of tris[(Z)-1-tert-butyl-4,4-dimethyl-3-oxo-pent-1-enoxy]manganese (0.358 g, 0.592 mmol) in dichloromethane (8 mL) was added dropwise, and the reaction was then stirred at 0° C. for 1 hour under oxygen atmosphere. The reaction was then warmed to room temperature and concentrated under reduced pressure. The resulting crude product was purified by flash chromatography on silica gel (0-100% ethyl acetate in petroleum ether) to provide the title compound: LCMS m/z 154.1 [M+H]⁺; ¹H NMR (400 MHz, CD₃OD) δ 8.15 (s, 1H), 7.92 (s, 1H), 1.52 (s, 6H).

### Step C. 2-(3-(2-bromo-5-fluoropyrimidin-4-yl)imidazo[1,2-a]pyrazin-6-yl)propan-2-ol

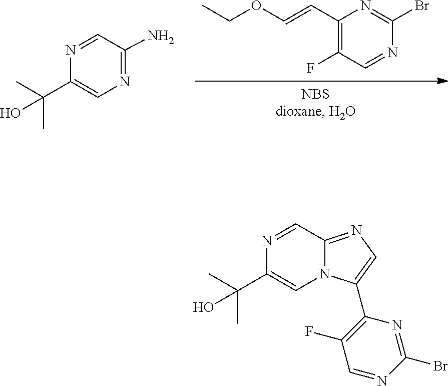

To a cooled 0° C. solution of 2-bromo-4-[(E)-2-ethoxyvinyl]-5-fluoro-pyrimidine (0.484 g, 1.96 mmol) in dioxane (30 mL) and water (10 mL) was added 1-bromopyrrolidine-2,5-dione (0.349 g, 1.96 mmol). The resulting reaction was stirred 30 minutes while warming to room temperature. 2-(5-Aminopyrazin-2-yl) propan-2-ol (0.250 g, 1.63 mmol) was then added, and the reaction was heated to 80° C. for 16 hours. The reaction was then cooled to room temperature, basified to pH ~9 by addition of aqueous sodium hydroxide solution (2 M, 2 mL), diluted with water (40 mL), and extracted with ethyl acetate (3×40 mL). The organic extracts were combined, washed with saturated aqueous sodium chloride solution (40 mL), dried over sodium sulfate, filtered, and concentrated under reduced pressure. The resulting crude product was purified by flash chromatography on silica gel (0-80% ethyl acetate in petroleum ether) to provide the title compound: LCMS m/z 351.9 [M+H]⁺; ¹H NMR (400 MHz, CD₃OD) δ 10.05 (d, J=1.4 Hz, 1H), 9.19 (d, J=1.3 Hz, 1H), 8.69 (d, J=3.3 Hz, 1H), 8.62 (d, J=3.8 Hz, 1H), 1.66 (s, 6H).

### Step D. tert-butyl (3S,4S)-3-fluoro-4-((5-fluoro-4-(6-(2-hydroxypropan-2-yl)imidazo[1,2-a]pyrazin-3-yl)pyrimidin-2-yl)amino)pyrrolidine-1-carboxylate

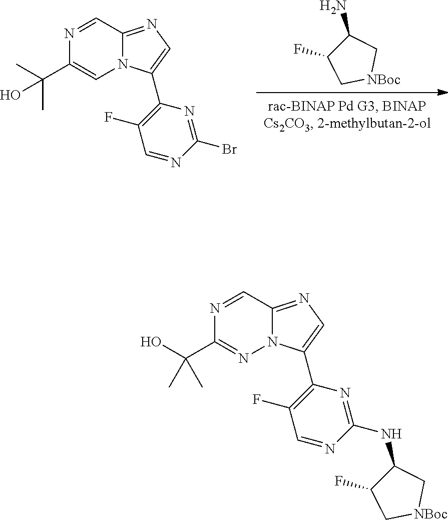

A mixture of 2-(3-(2-bromo-5-fluoropyrimidin-4-yl)imidazo[1,2-a]pyrazin-6-yl)propan-2-ol (0.050 g, 0.142 mmol), tert-butyl (3S,4S)-3-amino-4-fluoropyrrolidine-1-carboxylate (0.044 g, 0.213 mmol), methanesulfonato[2,2-bis(diphenylphosphino)-1,1-binaphthyl](2-amino-1,1-biphenyl-2-yl)palladium(II) (0.014 g, 0.014 mmol), (R)-(+)-2,2′-bis(diphenylphosphino)-1,1′-binaphthyl (0.009 g, 0.014 mmol), and cesium carbonate (0.139 g, 0.426 mmol) in 2-methylbutan-2-ol (3 mL) was purged with nitrogen, and was then heated at 80° C. for 16 hours under nitrogen atmosphere. The reaction was then cooled to room temperature and concentrated under reduced pressure to provide the title compound: LCMS m/z 476.1 [M+H]⁺.

### Step E. 2-(3-(5-fluoro-2-(((3S,4S)-4-fluoropyrrolidin-3-yl)amino)pyrimidin-4-yl)imidazo[1,2-a]pyrazin-6-yl)propan-2-ol

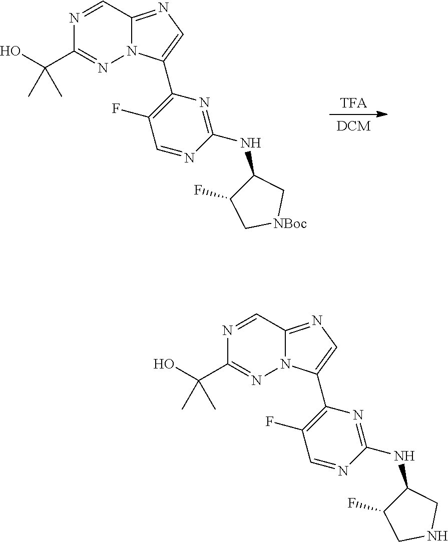

To a solution of tert-butyl (3S,4S)-3-fluoro-4-((5-fluoro-4-(6-(2-hydroxypropan-2-yl)imidazo[1,2-a]pyrazin-3-yl)pyrimidin-2-yl)amino)pyrrolidine-1-carboxylate (0.080 g, 0.168 mmol) in dichloromethane (3 mL) was added trifluoroacetic acid (0.770 g, 6.75 mmol, 0.50 mL). The resulting reaction was stirred at room temperature for 2 hours, and was then concentrated under reduced pressure. The crude product thus obtained was purified by HPLC (Phenomenex Luna C18 column, 5 micron, 75×30 mm; 1-30% acetonitrile in water containing 0.04% trifluoroacetic acid) to provide the title compound: LCMS m/z 376.1 [M+H]⁺; ¹H NMR (400 MHz, CD₃OD) δ 10.08 (d, J=1.4 Hz, 1H), 9.15 (d, J=1.4 Hz, 1H), 8.53 (d, J=3.6 Hz, 1H), 8.47 (d, J=3.5 Hz, 1H), 5.56-5.40 (d, J=50.4 Hz, 1H), 4.84-4.77 (m, 1H), 3.98 (dd, J=6.2, 12.7 Hz, 1H), 3.85-3.76 (m, 1H), 3.76-3.69 (m, 1H), 3.65 (dd, J=2.8, 12.8 Hz, 1H), 1.66 (d, J=1.5 Hz, 6H).

The compounds in Table 20 were all prepared using the synthetic procedures described in Example 21.

TABLE 20
Additional compounds prepared according to Example 21.

| Compound | | | |
|---|---|---|---|
| # | Structure | IUPAC Name | LCMS |
| 99a | 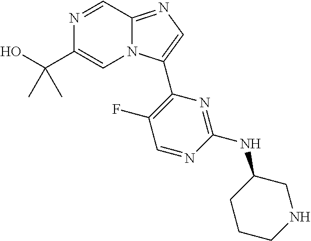 [SMILES: CC(C)(O)c1cn2c(-c3nc(N[C@@H]4CCCNC4)ncc3F)cnc2cn1] | (R)-2-(3-(5-fluoro-2- (piperidin-3- ylamino)pyrimidin-4- yl)imidazo[1,2- a]pyrazin-6-yl)propan-2- ol | 372.1 |
| 100a | 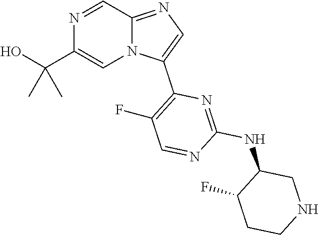 [SMILES: CC(C)(O)c1cn2c(-c3nc(N[C@H]4CNCC[C@@H]4F)ncc3F)cnc2cn1] | 2-(3-(5-fluoro-2- (((3S,4S)-4- fluoropiperidin-3- yl)amino)pyrimidin-4- yl)imidazo[1,2- a]pyrazin-6-yl)propan-2- ol | 390.1 |

### Example 22

### Exemplary Synthetic Procedure #20 (Compounds 101a-102a)

### Compound 101a, 5-chloro-4-(6-cyclopropyl-7-methoxyimidazo[1,2-b]pyridazin-3-yl)-N-((3S,4S)-4-fluoropyrrolidin-3-yl)pyrimidin-2-amine

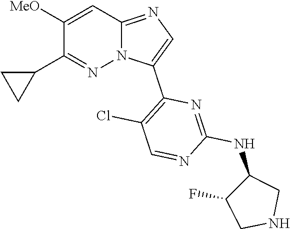 [SMILES: COc1cc2ncc(-c3nc(N[C@H]4CNC[C@@H]4F)ncc3Cl)n2nc1C1CC1]

### Step A. 3-(5-chloro-2-(methylthio)pyrimidin-4-yl)-6-cyclopropyl-7-methoxyimidazo[1,2-b]pyridazine

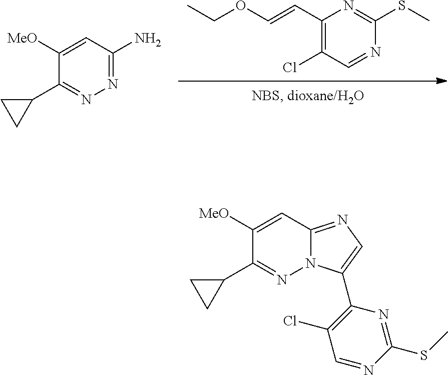

To a cooled 0° C. solution of 5-chloro-4-[(E)-2-ethoxyvinyl]-2-methylsulfanyl-pyrimidine (1.40 g, 6.05 mmol) in dioxane (15 mL) and water (5 mL) was added 1-bromopyrrolidine-2,5-dione (1 0.08 g, 6.05 mmol). The resulting reaction was stirred for 30 minutes while warming to room temperature. 6-Cyclopropyl-5-methoxy-pyridazin-3-amine (0.500 g, 3.03 mmol) and sodium bicarbonate (0.509 g, 6.05 mmol) were then added, and the reaction was heated to 80° C. for 2 hours. The reaction was then cooled to room temperature, basified to pH ~9 by addition of aqueous sodium hydroxide solution (2 M, 2 mL), diluted with water (10 mL), and extracted with ethyl acetate (3×10 mL). The organic extracts were combined, washed with saturated aqueous sodium chloride solution (10 mL), dried over sodium sulfate, filtered, and concentrated under reduced pressure. The resulting crude product was purified by flash chromatography on silica gel (0-60% ethyl acetate in petroleum ether) to provide the title compound: LCMS m/z 347.9 [M+H]⁺; ¹HNMR (400 MHz, CD₃OD) δ 8.67 (s, 1H), 8.09 (s, 1H), 7.32 (s, 1H), 4.05 (s, 3H), 2.59 (s, 3H), 2.51-2.44 (m, 1H), 1.13-1.03 (m, 4H).

### Step B. 3-(5-chloro-2-(methylsulfonyl)pyrimidin-4-yl)-6-cyclopropyl-7-methoxyimidazo[1,2-b]pyridazine

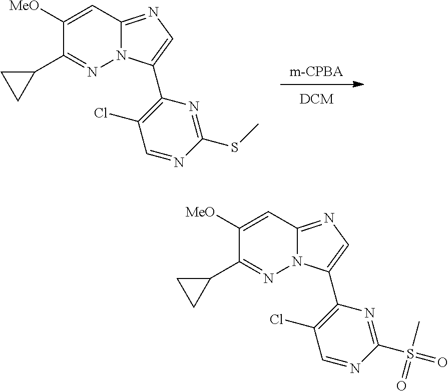

To a cooled 0° C. solution of 3-(5-chloro-2-methylsulfanyl-pyrimidin-4-yl)-6-cyclopropyl-7-methoxy-imidazo[1,2-b]pyridazine (0.380 g, 1.09 mmol) in dichloromethane (7 mL) was added in portions 3-chlorobenzenecarboperoxoic acid (0.444 g, 2.19 mmol, 85% purity). The resulting reaction was stirred for 2 hours while warming to room temperature. The reaction was then cooled to 0° C., quenched by addition of saturated aqueous sodium sulfite solution (10 mL), and extracted with ethyl acetate (3×10 mL). The organic extracts were combined, washed with saturated aqueous sodium chloride solution (2×10 mL), dried over sodium sulfite, filtered, and concentrated under reduced pressure. The resulting crude product was purified by flash chromatography on silica gel (0-60% ethyl acetate in petroleum ether) to provide the title compound: LCMS m/z 380.2 [M+H]⁺; ¹HNMR (400 MHz, CDCl₃) δ 8.94 (s, 1H), 8.51 (s, 1H), 7.32 (s, 1H), 4.08 (s, 3H), 3.45 (s, 3H), 2.58-2.42 (m, 1H), 1.38-1.27 (m, 2H), 1.20-1.11 (m, 2H).

### Step C. tert-butyl (3S,4S)-3-((5-chloro-4-(6-cyclopropyl-7-methoxyimidazo[1,2-b]pyridazin-3-yl)pyrimidin-2-yl)amino)-4-fluoropyrrolidine-1-carboxylate

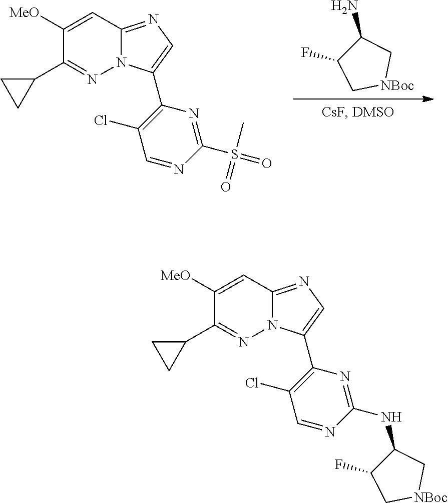

To a solution of 3-(5-chloro-2-methylsulfonyl-pyrimidin-4-yl)-6-cyclopropyl-7-methoxy-imidazo[1,2-b]pyridazine (0.050 g, 0.132 mmol) and tert-butyl (3S,4S)-3-amino-4-fluoro-pyrrolidine-1-carboxylate (0.040 g, 0.197 mmol) in dimethyl sulfoxide (3 mL) was added cesium fluoride (0.080 g, 0.527 mmol). The resulting reaction was heated at 100° C. for 8 hours. The reaction was then cooled to room temperature, diluted with water (5 mL), and extracted with ethyl acetate (3×10 mL). The organic extracts were combined, washed with saturated aqueous sodium chloride solution (5 mL), dried over sodium sulfate, filtered, and concentrated under reduced pressure. The resulting crude product was purified by flash chromatography on silica gel (0-100% ethyl acetate in petroleum ether) to provide the title compound: LCMS m/z 504.2 [M+H]⁺.

### Step D. 5-chloro-4-(6-cyclopropyl-7-methoxyimidazo[1,2-b]pyridazin-3-yl)-N-((3S,4S)-4-fluoropyrrolidin-3-yl)pyrimidin-2-amine

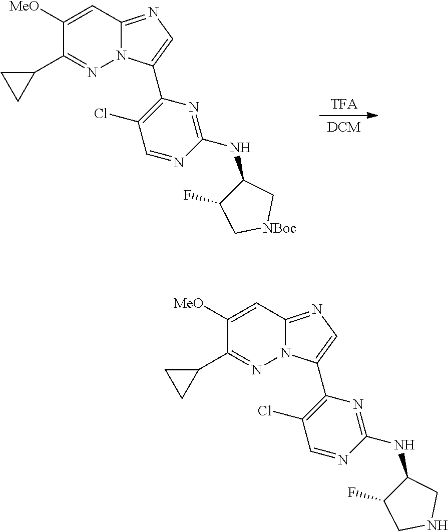

To a solution of tert-butyl (3S,4S)-3-[[5-chloro-4-(6-cyclopropyl-7-methoxy-imidazo[1,2-b]pyridazin-3-yl)pyrimidin-2-yl]amino]-4-fluoro-pyrrolidine-1-carboxylate (0.030 g, 0.060 mmol) in dichloromethane (2 mL) was added trifluoroacetic acid (0.921 g, 8.08 mmol, 0.600 mL). The resulting reaction was stirred at room temperature for 1.5 hours, and was then concentrated under reduced pressure. The crude product thus obtained was purified by HPLC (Phenomenex Luna C18 column, 75×30 mm, 3 micron, 1-30% acetonitrile in water containing 0.1% trifluoroacetic acid) to provide the title compound: LCMS m/z 404.0 [M+H]⁺; ¹HNMR (400 MHz, CD₃OD) δ 8.57 (s, 1H), 8.21 (d, J=3.9 Hz, 1H), 7.56 (d, J=4.6 Hz, 1H), 5.59-5.31 (d, J=50.4 Hz, 1H), 4.79-4.64 (m, 1H), 4.17-4.12 (s, 3H), 3.82-3.71 (m, 2H), 3.71-3.58 (m, 2H), 2.61-2.48 (m, 1H), 1.18-1.10 (m, 4H).

The compounds in Table 21 were all prepared using the synthetic procedures described in Example 22.

TABLE 21
Additional compounds prepared according to Example 22.

| Compound | | | |
|---|---|---|---|
| # | Structure | IUPAC Name | LCMS |
| 102a | 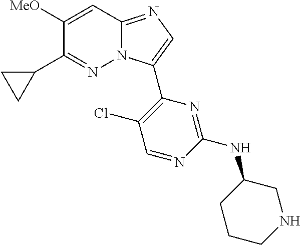 [SMILES: COc1cc2ncc(-c3nc(N[C@@H]4CCCNC4)ncc3Cl)n2nc1C1CC1] | (R)-5-chloro-4-(6- cyclopropyl-7- methoxyimidazo[1,2- b]pyridazin-3-yl)-N- (piperidin-3- yl)pyrimidin-2-amine | 400.1 |

### Example 23

### Exemplary Synthetic Procedure #21 (Compounds 103a-105a) Compound 103a, 5-chloro-4-(6-cyclopropyl-7-methoxyimidazo[1,2-b]pyridazin-3-yl)-N-((3S,4S)-4-fluoropiperidin-3-yl)pyrimidin-2-amine

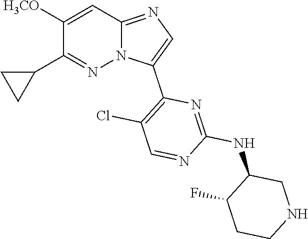 [SMILES: COc1cc2ncc(-c3nc(N[C@H]4CNCC[C@@H]4F)ncc3Cl)n2nc1C1CC1]

### Step A. 3-(2-bromo-5-chloropyrimidin-4-yl)-6-cyclopropyl-7-methoxyimidazo[1,2-b]pyridazine

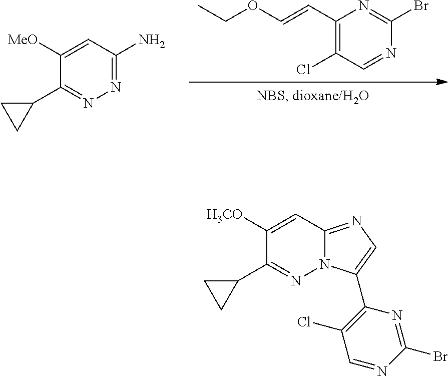

To a cooled 0° C. solution of 2-bromo-5-chloro-4-[(E)-2-ethoxyvinyl]pyrimidine (0.024 g, 0.092 mmol) in dioxane (3 mL) and water (1 mL) was added 1-bromopyrrolidine-2,5-dione (0.016 g, 0.092 mmol). The reaction was then stirred for 30 minutes while warming to room temperature. 6-Cyclopropyl-5-methoxy-pyridazin-3-amine (0.022 g, 0.077 mmol, TFA salt) was added, and the reaction was heated to 80° C. for 2 hours. The reaction was then cooled to room temperature, diluted with water (5 mL), and extracted with ethyl acetate (3×5 mL). The organic extracts were combined, washed with saturated aqueous sodium chloride solution (5 mL), dried over sodium sulfate, filtered, and concentrated under reduced pressure. The resulting crude product was purified by flash chromatography on silica gel (0-50% ethyl acetate in petroleum ether) to provide the title compound: LCMS m/z 380.0 [M+H]⁺.

### Step B. tert-butyl (3S,4S)-3-((5-chloro-4-(6-cyclopropyl-7-methoxyimidazo[1,2-b]pyridazin-3-yl)pyrimidin-2-yl)amino)-4-fluoropiperidine-1-carboxylate

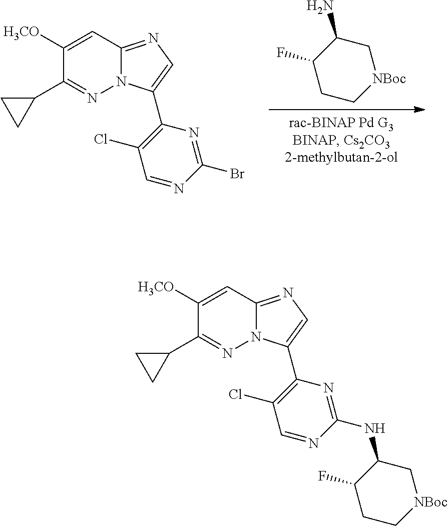

A mixture of 3-(2-bromo-5-chloro-pyrimidin-4-yl)-6-cyclopropyl-7-methoxy-imidazo[1,2-b]pyridazine (0.025 g, 0.066 mmol), tert-butyl (3S,4S)-3-amino-4-fluoro-piperidine-1-carboxylate (0.022 g, 0.099 mmol), methanesulfonato[2,2-bis(diphenylphosphino)-1,1-binaphthyl](2-amino-1,1-biphenyl-2-yl)palladium(II) (0.007 g, 0.007 mmol), (R)-(+)-2,2′-bis(diphenylphosphino)-1,1′-binaphthyl (0.006 g, 0.010 mmol), and cesium carbonate (0.054 g, 0.164 mmol) in 2-methylbutan-2-ol (3 mL) was purged with nitrogen, and was then heated at 80° C. for 4 hours under nitrogen atmosphere. The reaction was then cooled to room temperature, filtered, and concentrated under reduced pressure to provide the title compound: LCMS m/z 518.15 [M+H]⁻.

### Step C. 5-chloro-4-(6-cyclopropyl-7-methoxyimidazo[1,2-b]pyridazin-3-yl)-N-((3S,4S)-4-fluoropiperidin-3-yl)pyrimidin-2-amine

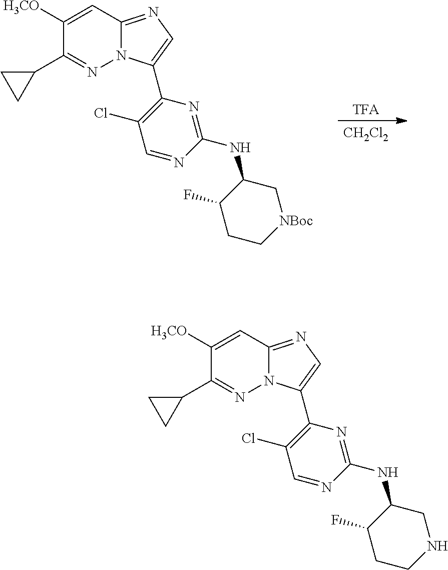 [SMILES: COc1cc2ncc(-c3nc(N[C@H]4CN(C(=O)OC(C)(C)C)CC[C@@H]4F)ncc3Cl)n2nc1C1CC1 ; "CH2Cl2" ; "TFA" ; COc1cc2ncc(-c3nc(N[C@H]4CNCC[C@@H]4F)ncc3Cl)n2nc1C1CC1]

To a solution of tert-butyl (3S,4S)-3-[[5-chloro-4-(6-cyclopropyl-7-methoxy-imidazo[1,2-b]pyridazin-3-yl)pyrimidin-2-yl]amino]-4-fluoro-piperidine-1-carboxylate (0.050 g, 0.097 mmol) in dichloromethane (2 mL) was added trifluoroacetic acid (0.50 mL). The resulting reaction was stirred at room temperature for 1 hour, and was then concentrated under reduced pressure. The crude product thus obtained was purified by HPLC (Phenomenex Luna C18 column, 3 micron, 80×30 mm; 1-25% acetonitrile in water containing 0.1% hydrochloric acid) to provide the title compound: LCMS m/z 418.0 [M+H]⁺; ¹HNMR (400 MHz, CD₃OD) δ 8.58 (s, 1H), 8.36 (s, 1H), 7.68 (s, 1H), 4.96-4.90 (m, 1H), 4.54-4.44 (m, 1H), 4.20 (s, 3H), 3.63-3.57 (m, 1H), 3.50-3.43 (m, 1H), 3.22-3.11 (m, 2H), 2.63-2.55 (m, 1H), 2.49-2.40 (m, 1H), 2.12-2.01 (m, 1H), 1.21-1.14 (m, 4H).

The compounds in Table 22 were all prepared using the synthetic procedures described in Example 23.

TABLE 22
Additional compounds prepared according to Example 23.

| Compound | | | |
|---|---|---|---|
| # | Structure | IUPAC Name | LCMS |
| 104a | 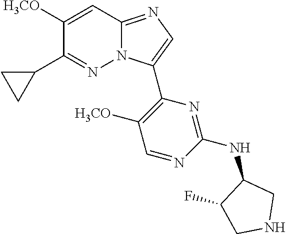 [SMILES: COc1cnc(N[C@H]2CNC[C@@H]2F)nc1-c1cnc2cc(OC)c(C3CC3)nn12] | 4-(6-cyclopropyl-7- methoxyimidazo[1,2- b]pyridazin-3-yl)-N- ((3S,4S)-4- fluoropyrrolidin-3-yl)-5- methoxypyrimidin-2- amine | 400.0 |
| 105a | 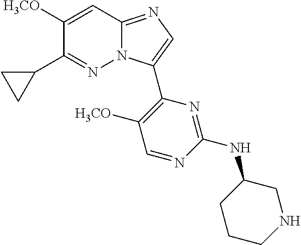 [SMILES: COc1cnc(N[C@@H]2CCCNC2)nc1-c1cnc2cc(OC)c(C3CC3)nn12] | (R)-4-(6-cyclopropyl-7- methoxyimidazo[1,2- b]pyridazin-3-yl)-5- methoxy-N-(piperidin-3- yl)pyrimidin-2-amine | 396.0 |

### Example 24

### Exemplary Synthetic Procedure #22 (Compounds 106a-108a) Compound 106a, 1-(3-(5-fluoro-2-(((3S,4S)-4-fluoropiperidin-3-yl)amino)pyrimidin-4-yl)-7-methoxyimidazo[1,2-b]pyridazin-6-yl)pyrrolidin-2-one

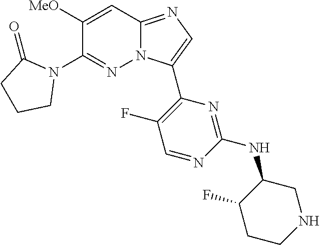 [SMILES: COc1cc2ncc(-c3nc(N[C@H]4CNCC[C@@H]4F)ncc3F)n2nc1N1CCCC1=O]

### Step A. 4-bromo-N-(4-bromobutanoyl)-N-(6-chloro-4-methoxypyridazin-3-yl)butanamide

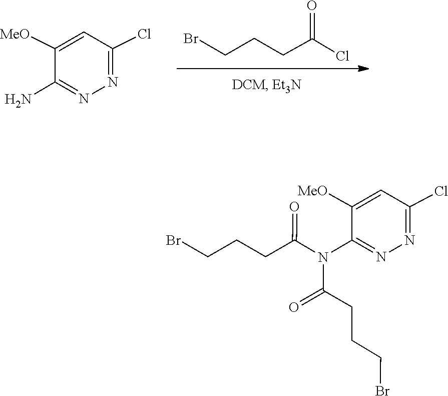 [SMILES: COc1cc(Cl)nnc1N ; O=C(Cl)CCCBr ; COc1cc(Cl)nnc1N(C(=O)CCCBr)C(=O)CCCBr]

To a cooled 0° C. solution of 6-chloro-4-methoxy-pyridazin-3-amine (5.00 g, 31.3 mmol) in dichloromethane (100 mL) were added triethylamine (4.76 g, 47.0 mmol, 6.54 mL) and 4-bromobutanoyl chloride (11.6 g, 62.7 mmol, 7.26 mL). The resulting reaction was stirred at 0° C. for 30 minutes, and was then stirred for an additional 15 hours at room temperature. The reaction was then diluted with water (100 mL) and extracted with dichloromethane (3×100 mL). The organic extracts were combined, washed with saturated aqueous sodium chloride solution (100 mL), dried over sodium sulfate, filtered, and concentrated under reduced pressure. The resulting crude product was purified by flash chromatography on silica gel (5-60% ethyl acetate in petroleum ether) to provide the title compound: LCMS m/z 457.8 [M+H]⁺.

### Step B. 1-(6-chloro-4-methoxypyridazin-3-yl)pyrrolidin-2-one

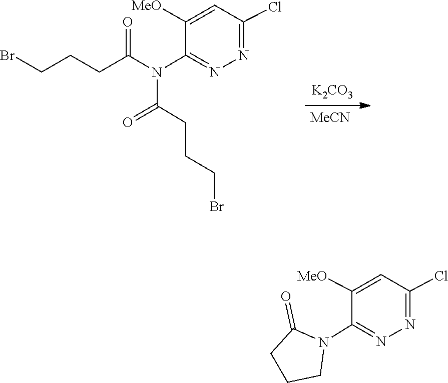

To a solution of 4-bromo-N-(4-bromobutanoyl)-N-(6-chloro-4-methoxy-pyridazin-3-yl)butanamide (2.00 g, 4.37 mmol) in water (5 mL) and methanol (25 mL) was added potassium carbonate (1.21 g, 8.74 mmol). The resulting reaction was stirred at room temperature for 4 hours, and was then concentrated under reduced pressure. The resulting crude product was purified by flash chromatography on silica gel (5-60% ethyl acetate in petroleum ether) to provide the title compound: LCMS m/z 227.9 [M+H]⁺; ¹H NMR (400 MHz, CD₃OD) δ 7.54 (s, 1H), 4.01-3.99 (m, 3H), 3.98-3.94 (m, 2H), 2.62-2.56 (m, 2H), 2.36-2.21 (m, 2H).

### Step C. tert-butyl (5-methoxy-6-(2-oxopyrrolidin-1-yl)pyridazin-3-yl)carbamate

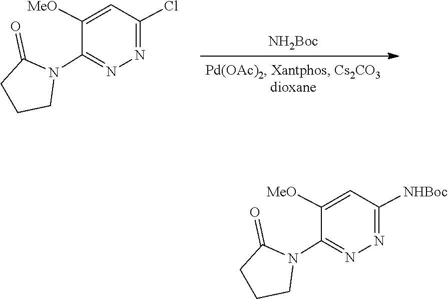 [SMILES: COc1cc(Cl)nnc1N1CCCC1=O ; "Pd(OAc)2, Xantphos, Cs2CO3dioxane" ; COc1cc(NC(=O)OC(C)(C)C)nnc1N1CCCC1=O ; "NH2Boc"]

A mixture of 1-(6-chloro-4-methoxy-pyridazin-3-yl)pyrrolidin-2-one (3.00 g, 13.2 mmol), tert-butyl carbamate (5.40 g, 46.1 mmol), [2-(2-aminophenyl)phenyl]-methylsulfonyloxypalladiumditert-butyl-[2-(2,4,6-triisopropylphenyl)phenyl]phosphane (1.05 g, 1.32 mmol), and sodium tert-butoxide solution (2 M in THF, 13.2 mL) in dioxane (80 mL) was purged with nitrogen, and was then heated at 100° C. for 16 hours under nitrogen atmosphere. The reaction was then cooled to room temperature, diluted with water (80 mL), and extracted with ethyl acetate (3×50 mL). The organic extracts were combined, washed with saturated aqueous sodium chloride solution (80 mL), dried over sodium sulfate, filtered, and concentrated under reduced pressure. The resulting crude product was purified by flash chromatography on silica gel (5-70% ethyl acetate in petroleum ether) to provide the title compound: LCMS m/z 309.2 [M+H]⁺; ¹H NMR (400 MHz, CD₃OD) δ 7.96 (s, 1H), 3.97 (s, 3H), 3.89 (t, J=7.0 Hz, 2H), 2.58 (t, J=8.1 Hz, 2H), 2.28 (q, J=7.5 Hz, 2H), 1.56 (s, 9H).

### Step D. 1-(6-amino-4-methoxypyridazin-3-yl)pyrrolidin-2-one

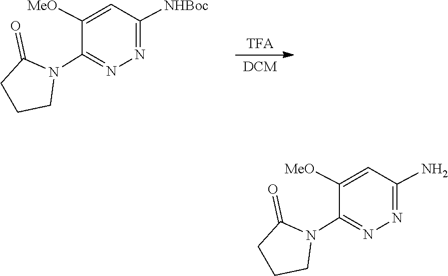

To a solution of tert-butyl N-[5-methoxy-6-(2-oxopyrrolidin-1-yl)pyridazin-3-yl]carbamate (1.50 g, 4.86 mmol) in dichloromethane (8 mL) was added trifluoroacetic acid (10.8 g, 94.2 mmol, 4.00 mL). The resulting reaction was stirred at room temperature for 30 minutes, and was then cooled to 0° C. and neutralized to pH ~7 by slow addition of saturated aqueous sodium bicarbonate solution (10 mL). The reaction mixture was then filtered and concentrated under reduced pressure to provide the title compound: LCMS m/z 209.2 [M+H]⁺.

### Step E. 1-(3-(2-bromo-5-fluoropyrimidin-4-yl)-7-methoxyimidazo[1,2-b]pyridazin-6-yl)pyrrolidin-2-one

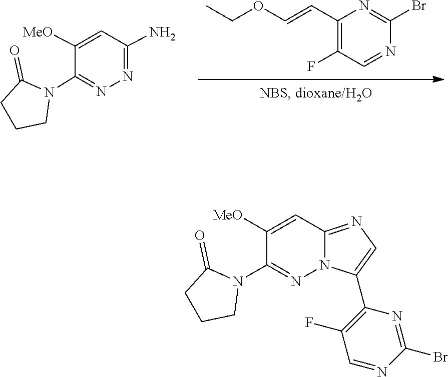

To a cooled 0° C. solution of (E)-2-bromo-4-(2-ethoxyvinyl)-5-fluoropyrimidine (0.890 g, 3.60 mmol) in dioxane (9 mL) and water (3 mL) was added 1-bromopyrrolidine-2,5-dione (0.641 g, 3.60 mmol). The reaction was then stirred 30 minutes while warming to room temperature. 1-(6-Amino-4-methoxypyridazin-3-yl)pyrrolidin-2-one (0.500 g, 2.40 mmol) and sodium bicarbonate (0.403 g, 4.80 mmol) were added, and the reaction was then heated at 80° C. for 16 hours. The reaction was then cooled to room temperature, diluted with water (10 mL), and extracted with ethyl acetate (3×10 mL). The organic extracts were combined, washed with saturated aqueous sodium chloride solution (10 mL), dried over sodium sulfate, filtered, and concentrated under reduced pressure. The resulting crude product was purified by flash chromatography on silica gel (0-10% methanol in ethyl acetate) to provide the title compound: LCMS m/z 407.0 [M+H]⁺.

### Step F. tert-butyl (R)-3-((5-fluoro-4-(7-methoxy-6-(2-oxopyrrolidin-1-yl)imidazo[1,2-b]pyridazin-3-yl)pyrimidin-2-yl)amino)piperidine-1-carboxylate

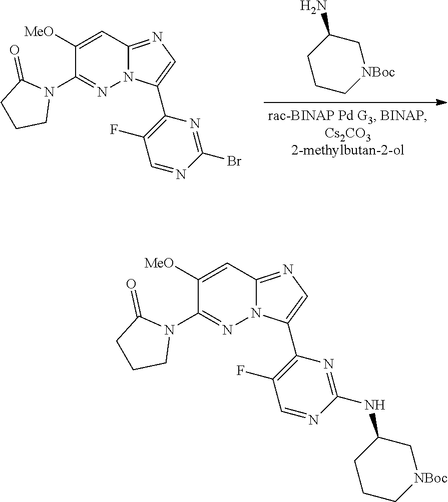

A mixture of 1-(3-(2-bromo-5-fluoropyrimidin-4-yl)-7-methoxyimidazo[1,2-b]pyridazin-6-yl)pyrrolidin-2-one (0.050 g, 0.123 mmol), tert-butyl (R)-3-aminopiperidine-1-carboxylate (0.030 g, 0.147 mmol), methanesulfonato[2,2-bis(diphenylphosphino)-1,1-binaphthyl](2-amino-1,1-biphenyl-2-yl)palladium(II) (0.012 g, 0.012 mmol), (R)-(+)-2,2′-bis(diphenylphosphino)-1,1′-binaphthyl (0.008 g, 0.012 mmol), and cesium carbonate (0.120 g, 0.368 mmol) in 2-methylbutan-2-ol (3 mL) was purged with nitrogen, and was then heated at 80° C. for 16 hours under nitrogen atmosphere. The reaction was then cooled to room temperature, filtered, and concentrated under reduced pressure to provide the title compound: LCMS m/z 527.3 [M+H]⁺.

### Step G. (R)-1-(3-(5-fluoro-2-(piperidin-3-ylamino)pyrimidin-4-yl)-7-methoxyimidazo[1,2-b]pyridazin-6-yl)pyrrolidin-2-one

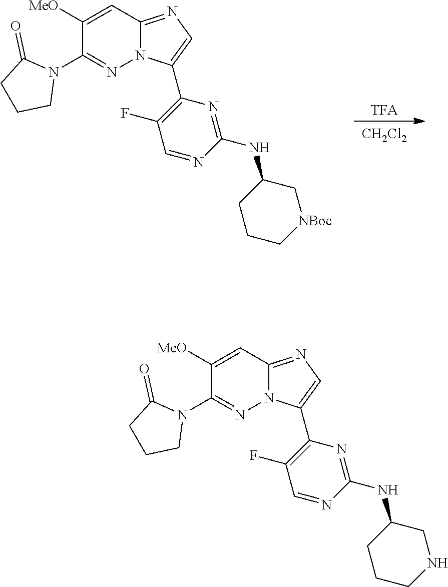 [SMILES: COc1cc2ncc(-c3nc(N[C@@H]4CCCN(C(=O)OC(C)(C)C)C4)ncc3F)n2nc1N1CCCC1=O ; "CH2Cl2" ; "TFA" ; COc1cc2ncc(-c3nc(N[C@@H]4CCCNC4)ncc3F)n2nc1N1CCCC1=O]

To a solution of tert-butyl (R)-3-((5-fluoro-4-(7-methoxy-6-(2-oxopyrrolidin-1-yl)imidazo[1,2-b]pyridazin-3-yl)pyrimidin-2-yl)amino)piperidine-1-carboxylate (0.130 g, 0.247 mmol) in dichloromethane (2 mL) was added trifluoroacetic acid (2.99 g, 26.3 mmol, 1.95 mL). The resulting reaction was stirred at room temperature for 1 hour, and was then concentrated under reduced pressure. The crude product thus obtained was purified by HPLC (Waters Xbridge Prep OBD C18 column, 5 micron, 150×40 mm; 1-30% acetonitrile in water containing 0.04% NH₄CO₃) to provide the title compound: LCMS m/z 427.1 [M+H]⁺; ¹H NMR (400 MHz, CD₃OD) δ 8.32 (d, J=2.9 Hz, 1H), 8.15 (d, J=2.1 Hz, 1H), 7.60 (s, 1H), 4.68-4.55 (m, 1H), 4.05 (s, 3H), 3.98-3.92 (m, 2H), 3.30-3.24 (m, 1H), 3.05-2.97 (m, 1H), 2.70-2.55 (m, 4H), 2.39-2.26 (m, 2H), 2.10-2.07 (m, 1H), 1.92-1.81 (m, 1H), 1.71-1.56 (m, 2H).

The compounds in Table 23 were all prepared using the synthetic procedures described in Example 24.

TABLE 23
Additional compounds prepared according to Example 24.

| Compound | | | |
|---|---|---|---|
| # | Structure | IUPAC Name | LCMS |
| 107a | 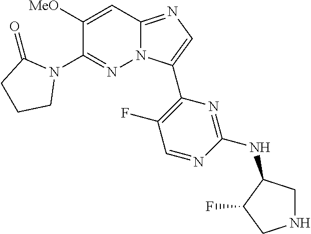 [SMILES: COc1cc2ncc(-c3nc(N[C@H]4CNC[C@@H]4F)ncc3F)n2nc1N1CCCC1=O] | 1-(3-(5-fluoro-2- (((3S,4S)-4- fluoropyrrolidin-3- yl)amino)pyrimidin-4- yl)-7- methoxyimidazo[1,2- b]pyridazin-6- yl)pyrrolidin-2-one | 431.0 |
| 108a | 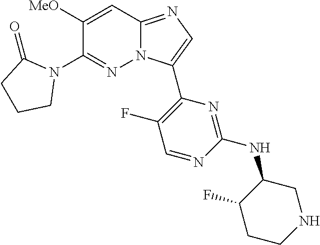 [SMILES: COc1cc2ncc(-c3nc(N[C@H]4CNCC[C@@H]4F)ncc3F)n2nc1N1CCCC1=O] | 1-(3-(5-fluoro-2- (((3S,4S)-4- fluoropiperidin-3- yl)amino)pyrimidin-4- yl)-7- methoxyimidazo[1,2- b]pyridazin-6- yl)pyrrolidin-2-one | 445.1 |

### Example 25

### Exemplary Synthetic Procedure #23 (Compound 109a)

### Compound 109a, N-((3S,4S)-4-fluoropiperidin-3-yl)-4-(7-methoxy-6-(oxetan-2-yl)imidazo[1,2-b]pyridazin-3-yl)pyrimidin-2-amine

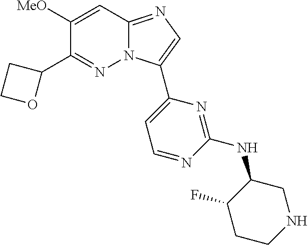 [SMILES: COc1cc2ncc(-c3ccnc(N[C@H]4CNCC[C@@H]4F)n3)n2nc1C1CCO1]

### Step A. 6-chloro-4-methoxy-3-(oxetan-2-yl)pyridazine

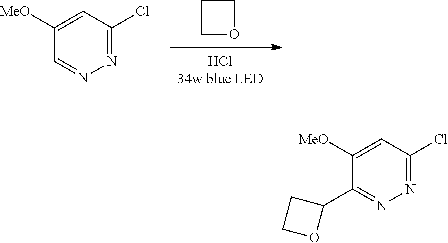

A mixture of 3-chloro-5-methoxy-pyridazine (1.00 g, 6.92 mmol), oxetane (4.02 g, 69.2 mmol, 4.50 mL), hydrogen peroxide (10.2 g, 89.9 mmol, 8.64 mL, 30% solution), and hydrochloric acid (4 M, 0.865 mL) in acetonitrile (20 mL) was purged with argon, and was then stirred and irradiated with two 40 W LEDs (vials approximately 6 cm away from the light source) at room temperature for 16 hours. The reaction was then cooled to 0° C., quenched by addition of saturated aqueous Na₂S₆O₄ solution (90 mL), and extracted with ethyl acetate (3×80 mL). The organic layers were combined, washed with saturated aqueous sodium chloride solution (3×80 mL), dried over anhydrous sodium sulfate, filtered, and concentrated under reduced pressure. The resulting crude product was purified by flash chromatography on silica gel (5-30% ethyl acetate in petroleum ether) to provide the title compound: LCMS m/z 203.1 [M+H]⁺; ¹H NMR (400 MHz, CD₃OD) δ 7.45 (s, 1H), 6.16 (dd, J=6.9, 8.3 Hz, 1H), 4.84-4.65 (m, 2H), 4.00 (s, 3H), 3.24-2.99 (m, 3H), 2.12-1.74 (m, 2H).

### Step B. tert-butyl (5-methoxy-6-(oxetan-2-yl)pyridazin-3-yl)carbamate

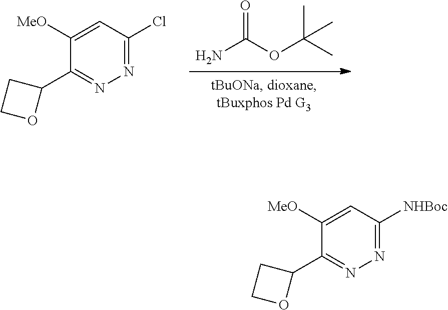

A mixture of 6-chloro-4-methoxy-3-(oxetan-2-yl)pyridazine (1.30 g, 6.48 mmol), tert-butyl carbamate (2.66 g, 22.7 mmol), sodium tert-butoxide solution (2 M in THF, 6.48 mL), and [2-(2-aminophenyl)phenyl]-methylsulfonyloxypalladium ditert-butyl-[2-(2,4,6-triisopropylphenyl)phenyl]phosphane (0.515 g, 0.648 mmol) in dioxane (30 mL) was purged with nitrogen, and was then heated at 100° C. for 16 hours under nitrogen atmosphere. The reaction was then cooled to room temperature, diluted with water (20 mL), and extracted with ethyl acetate (3×30 mL). The organic extracts were combined, washed with saturated aqueous sodium chloride solution (20 mL), dried over sodium sulfate, filtered, and concentrated under reduced pressure. The resulting crude product was purified by flash chromatography on silica gel (5-30% ethyl acetate in petroleum ether) to provide the title compound: LCMS m/z 282.2 [M+H]⁺; ¹H NMR (400 MHz, CD30D) 5 7.87 (s, 1H), 6.14 (t, J=7.6 Hz, 1H), 5.49 (s, 1H), 4.86-4.80 (m, 1H), 4.78-4.69 (m, 1H), 4.16-4.04 (m, 1H), 3.98 (s, 3H), 3.24-2.95 (m, 2H), 1.56 (s, 9H).

### Step C. 5-methoxy-6-(oxetan-2-yl)pyridazin-3-amine

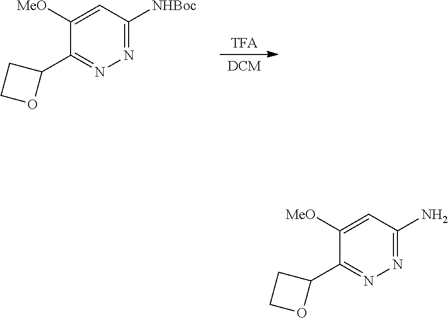

To a solution of tert-butyl N-[5-methoxy-6-(oxetan-2-yl)pyridazin-3-yl]carbamate (0.530 g, 1.88 mmol) in dichloromethane (5 mL) was added trifluoroacetic acid (4.61 g, 40.4 mmol, 3.0 mL). The resulting reaction was stirred at room temperature for 2 hours, and was then concentrated under reduced pressure. The crude product thus obtained was diluted with ethanol (10 mL) and basified to pH ~9 by addition of solid sodium bicarbonate. The resulting mixture was filtered and concentrated under reduced pressure to provide the title compound: LCMS m/z 182.2 [M+H]⁺.

### Step D. 3-(2-chloropyrimidin-4-yl)-7-methoxy-6-(oxetan-2-yl)imidazo[1,2-b]pyridazine

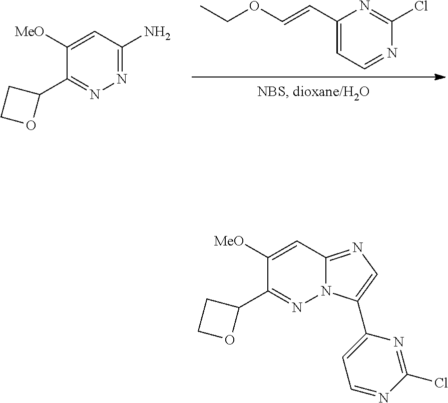 [SMILES: COc1cc(N)nnc1C1CCO1 ; CCO/C=C/c1ccnc(Cl)n1 ; "NBS, dioxane/H2O" ; COc1cc2ncc(-c3ccnc(Cl)n3)n2nc1C1CCO1]

To a cooled 0° C. solution of 2-chloro-4-[(E)-2-ethoxyvinyl]pyrimidine (1.12 g, 6.07 mmol) in dioxane (9 mL) and H₂O (3 mL) was added 1-bromopyrrolidine-2,5-dione (1.18 g, 6.62 mmol). The resulting reaction was stirred for 30 minutes while warming to room temperature. 5-Methoxy-6-(oxetan-2-yl)pyridazin-3-amine (1.00 g, 5.52 mmol) and sodium bicarbonate (0.927 g, 11.04 mmol) were then added, and the reaction was heated to 80° C. for 16 hours. The reaction was then cooled to room temperature, diluted with water (10 mL), and extracted with ethyl acetate (3×10 mL). The organic extracts were combined, washed with saturated aqueous sodium chloride solution (10 mL), dried over sodium sulfate, filtered, and concentrated under reduced pressure. The resulting crude product was purified by flash chromatography on silica gel (10-100% ethyl acetate in petroleum ether) to provide the title compound: LCMS m/z 317.9 [M+H]⁺.

### Step E. tert-butyl (3S,4S)-4-fluoro-3-((4-(7-methoxy-6-(oxetan-2-yl)imidazo[1,2-b]pyridazin-3-yl)pyrimidin-2-yl)amino)piperidine-1-carboxylate

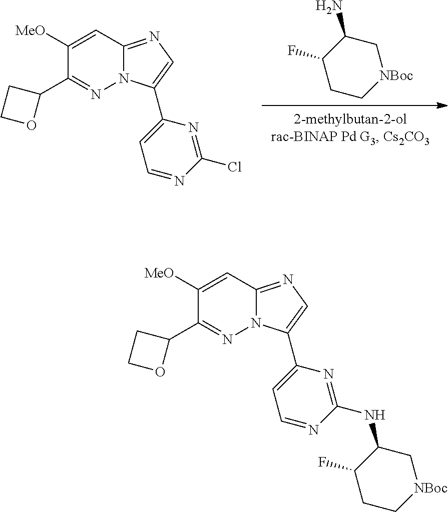

A mixture of 3-(2-chloropyrimidin-4-yl)-7-methoxy-6-(oxetan-2-yl)imidazo[1,2-b]pyridazine (0.010 g, 0.031 mmol), tert-butyl (3 S,4S)-3-amino-4-fluoro-piperidine-1-carboxylate (0.010 g, 0.047 mmol), cesium carbonate (0.031 g, 0.094 mmol), methanesulfonato[2,2-bis(diphenylphosphino)-1,1-binaphthyl](2-amino-1,1-biphenyl-2-yl)palladium(II) (0.003 g, 0.003 mmol) and (R)-(+)-2,2′-bis(diphenylphosphino)-1,1′-binaphthyl (0.003 g, 0.005 mmol) in 2-methylbutan-2-ol (2 mL) was purged with nitrogen, and was then heated at 80° C. for 4 hours under nitrogen atmosphere. The reaction was then cooled to room temperature and concentrated under reduced pressure to provide the title compound: LCMS m/z 500.5 [M+H]⁺.

### Step F. N-((3S,4S)-4-fluoropiperidin-3-yl)-4-(7-methoxy-6-(oxetan-2-yl)imidazo[1,2-b]pyridazin-3-yl)pyrimidin-2-amine

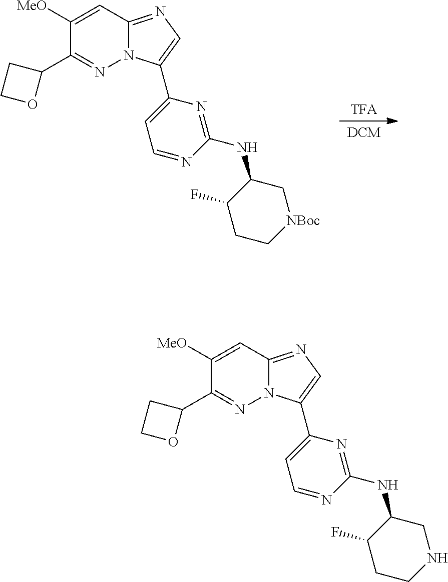

To a solution of tert-butyl (3S,4S)-4-fluoro-3-[[4-[7-methoxy-6-(oxetan-2yl)imidazo[1,2-b]pyridazin-3-yl]pyrimidin2-yl]amino]piperidine-1-carboxylate (0.020 g, 0.040 mmol) in dichloromethane (2 mL) was added trifluoroacetic acid (1.54 g, 13.5 mmol, 1.00 mL). The resulting reaction was stirred at room temperature for 30 minutes, and was then concentrated under reduced pressure. The crude product thus obtained was purified by HPLC (Waters X bridge BEH C18 column, 3 micron, 100×30 mm; 15-45% acetonitrile in water containing 0.1% ammonium bicarbonate) to provide the title compound: LCMS m/z 400.1 [M+H]⁺; ¹HNMR (400 MHz, CD₃OD) δ 8.46 (s, 1H), 8.38 (d, J=5.3 Hz, 1H), 7.98 (dd, J=1.6, 5.3 Hz, 1H), 7.48 (s, 1H), 6.21 (t, J=6.4 Hz, 1H), 4.84-4.78 (m, 2H), 4.70-4.59 (m, 1H), 4.29-4.18 (m, 1H), 4.02 (s, 3H), 3.41-3.35 (m, 2H), 3.18 (m, 2H), 2.81-2.47 (m, 2H), 2.20 (m, 1H), 1.79 (m, 1H).

### Example 26

### Exemplary Synthetic Procedure #24 (Compounds 110a-112a)

### Compound 110a, (R)-2-(3-(5-fluoro-2-(piperidin-3-ylamino)pyrimidin-4-yl)-7-methoxyimidazo[1,2-b]pyridazin-6-yl)isothiazolidine 1,1-dioxide

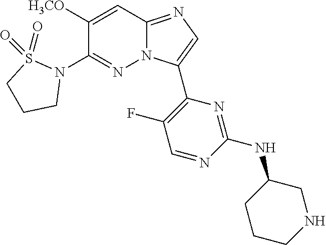 [SMILES: COc1cc2ncc(-c3nc(N[C@@H]4CCCNC4)ncc3F)n2nc1N1CCCS1(=O)=O]

### Step A. 2-(6-chloro-4-methoxypyridazin-3-yl)isothiazolidine 1,1-dioxide

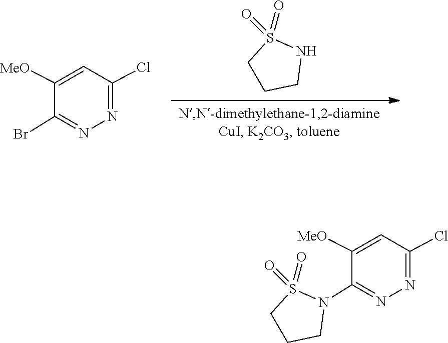

A mixture of 3-bromo-6-chloro-4-methoxypyridazine (1.00 g, 4.48 mmol), isothiazolidine 1,1-dioxide (0.813 g, 6.71 mmol), copper(I)iodide (0.213 g, 1.12 mmol), potassium carbonate (1.24 g, 8.95 mmol), and N′,N′-dimethylethane-1,2-diamine (0.197 g, 2.24 mmol, 0.245 mL) in toluene (20 mL) was purged with nitrogen, and was then heated at 100° C. for 6 hours under nitrogen atmosphere. The reaction was then cooled to room temperature, diluted with water (20 mL), and extracted with ethyl acetate (3×20 mL). The organic extracts were combined, washed with saturated aqueous sodium chloride solution (20 mL), dried over sodium sulfate, filtered, and concentrated under reduced pressure. The resulting crude product was purified by flash chromatography on silica gel (0-80% ethyl acetate in petroleum ether) to provide the title compound: LCMS m/z 263.8 [M+H]⁺; ¹H NMR (400 MHz, CD₃OD) δ 7.29 (s, 1H), 4.03-4.07 (t, J=6.6 Hz, 2H), 3.90 (s, 3H), 3.28 (t, J=7.0 Hz, 2H), 2.43-2.51 (m, 2H).

### Step B. tert-butyl (6-(1,1-dioxidoisothiazolidin-2-yl)-5-methoxypyridazin-3-yl)carbamate

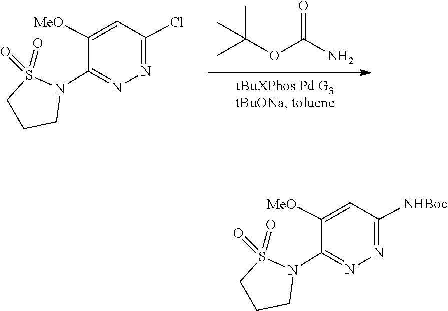

A mixture of 2-(6-chloro-4-methoxypyridazin-3-yl)isothiazolidine 1,1-dioxide (4.50 g, 17.1 mmol), tert-butyl carbamate (2.40 g, 20.5 mmol), [2-(2-aminophenyl)phenyl]-methylsulfonyloxypalladium ditertbutyl[2(2,4,6triisopropylphenyl)phenyl]phosphane (1.36 g, 1.71 mmol), and sodium tert-butoxide solution (2 M in THF, 17.1 mL) in dioxane (70 mL) was purged with nitrogen, and was then heated at 100° C. for 2 hours under nitrogen atmosphere. The reaction was then cooled to room temperature, diluted with water (40 mL), and extracted with ethyl acetate (3×40 mL). The organic extracts were combined, washed with saturated aqueous sodium chloride solution (40 mL), dried over anhydrous sodium sulfate, filtered, and concentrated under reduced pressure. The resulting crude product was purified by flash chromatography on silica gel (0-70% ethyl acetate in petroleum ether) to provide the title compound: LCMS m/z 345.0 [M+H]⁺.

### Step D. 2-(6-amino-4-methoxypyridazin-3-yl)isothiazolidine 1,1-dioxide

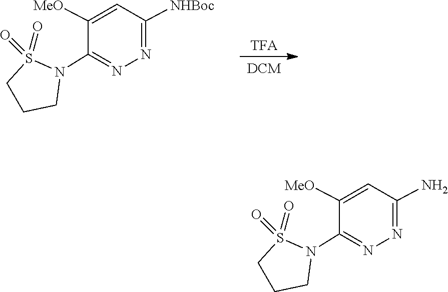

To a solution of tert-butyl (6-(1,1-dioxidoisothiazolidin-2-yl)-5-methoxypyridazin-3-yl)carbamate (2.50 g, 7.26 mmol) in dichloromethane (60 mL) was added trifluoroacetic acid (30.7 g, 269 mmol, 20.0 mL). The resulting reaction was stirred at room temperature for 2 hours, and was then concentrated under reduced pressure to provide the title compound: LCMS m/z 245.1 [M+H]⁺.

### Step E. 1-(3-(2-bromo-5-fluoropyrimidin-4-yl)-7-methoxyimidazo[1,2-b]pyridazin-6-yl)pyrrolidin-2-one

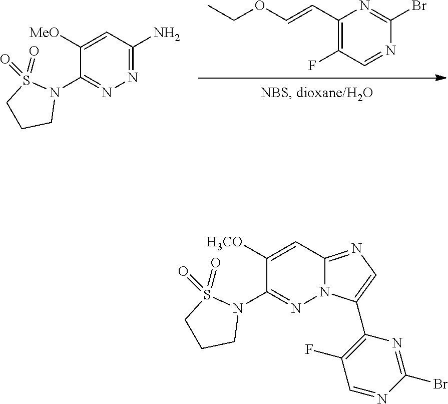

To a cooled 0° C. solution of (E)-2-bromo-4-(2-ethoxyvinyl)-5-fluoropyrimidine (0.303 g, 1.23 mmol) in dioxane (1.5 mL) and water (0.5 mL) was added 1-bromopyrrolidine-2,5-dione (0.219 g, 1.23 mmol). The resulting reaction was stirred for 30 minutes while warming to room temperature. 2-(6-Amino-4-methoxypyridazin-3-yl)isothiazolidine-1,1-dioxide (0.200 g, 0.819 mmol) and sodium bicarbonate (0.138 g, 1.64 mmol) were then added, and the reaction was heated to 80° C. for 16 hours. The reaction was then cooled to room temperature, basified to pH ~9 by addition of aqueous sodium hydroxide solution (2 M, 2 mL), diluted with water (10 mL), and extracted with ethyl acetate (3×5 mL). The organic extracts were combined, washed with saturated aqueous sodium chloride solution (10 mL), dried over sodium sulfate, filtered, and concentrated under reduced pressure. The resulting crude product was purified by flash chromatography on silica gel (0-70% ethyl acetate in petroleum ether) to provide the title compound: LCMS m/z 432.9 [M+H]⁺; ¹H NMR (400 MHz, CD₃OD) δ 8.52 (d, J=3.0 Hz, 1H), 8.19 (br d, J=2.4 Hz, 1H), 7.41 (s, 1H), 4.16 (t, J=6.6 Hz, 2H), 3.98-3.95 (s, 3H), 3.38-3.28 (m, 3H), 2.61-2.51 (m, 2H).

### Step F. tert-butyl (R)-3-((4-(6-(1,1-dioxidoisothiazolidin-2-yl)-7-methoxyimidazo[1,2-b]pyridazin-3-yl)-5-fluoropyrimidin-2-yl)amino)piperidine-1-carboxylate 1,1-dioxide

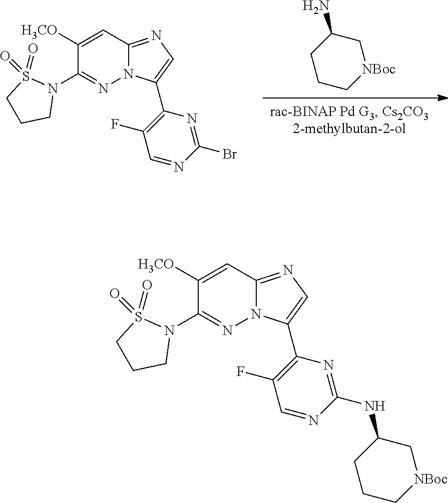

A mixture of 2-(3-(2-bromo-5-fluoropyrimidin-4-yl)-7-methoxyimidazo[1,2-b]pyridazin-6-yl)isothiazolidine 1,1-dioxide (0.070 g, 0.158 mmol), tert-butyl (R)-3-aminopiperidine-1-carboxylate (0.047 g, 0.237 mmol), methanesulfonato[2,2-bis(diphenylphosphino)-1,1-binaphthyl](2-amino-1,1-biphenyl-2-yl)palladium(II) (0.016 g, 0.016 mmol), (R)-(+)-2,2′-bis(diphenylphosphino)-1,1′-binaphthyl (0.010 g, 0.016 mmol), and cesium carbonate (0.154 g, 0.474 mmol) in 2-methylbutan-2-ol (3 mL) was purged with nitrogen, and was then stirred at 80° C. for 2 hours under nitrogen atmosphere. The reaction mixture was then cooled to room temperature, filtered, and concentrated under reduced pressure to provide the title compound: LCMS m/z 563.2 [M+H]⁺.

### Step G. (R)-2-(3-(5-fluoro-2-(piperidin-3-ylamino)pyrimidin-4-yl)-7-methoxyimidazo[1,2-b]pyridazin-6-yl)isothiazolidine 1,1-dioxide

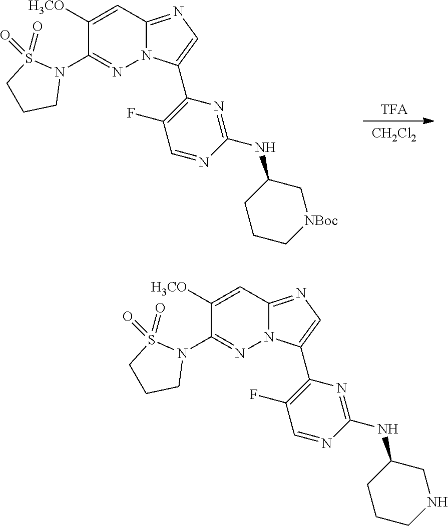 [SMILES: COc1cc2ncc(-c3nc(N[C@@H]4CCCN(C(=O)OC(C)(C)C)C4)ncc3F)n2nc1N1CCCS1(=O)=O ; "CH2Cl2" ; COc1cc2ncc(-c3nc(N[C@@H]4CCCNC4)ncc3F)n2nc1N1CCCS1(=O)=O ; "TFA"]

To a solution of tert-butyl (R)-3-((4-(6-(1,1-dioxidoisothiazolidin-2-yl)-7-methoxyimidazo[1,2-b]pyridazin-3-yl)-5-fluoropyrimidin-2-yl)amino)piperidine-1-carboxylate (0.080 g, 0.142 mmol) in dichloromethane (3 mL) was added trifluoroacetic acid (0.50 mL). The resulting reaction was stirred at room temperature for 2 hours, and was then concentrated under reduced pressure. The crude product thus obtained was purified by HPLC (Phenomenex Luna C18 column, 5 micron, 75×30 mm; 1-30% acetonitrile in water containing 0.04% trifluoroacetic acid) to provide the title compound: LCMS m/z 463.0 [M+H]⁺; ¹H NMR (400 MHz, CD₃OD) δ 8.42 (d, J=2.8 Hz, 1H), 8.16 (br s, 1H), 7.59 (s, 1H), 4.31-4.21 (m, 1H), 4.19-4.11 (m, 2H), 4.10 (s, 3H), 3.58 (br dd, J=3.5, 12.3 Hz, 1H), 3.44 (t, J=7.1 Hz, 2H), 3.32-3.29 (m, 1H), 3.05 (br dd, J=9.6, 11.9 Hz, 2H), 2.61-2.58 (m, 2H), 2.17-2.05 (m, 2H), 1.92-1.74 (m, 2H).

The compounds in Table 25 were all prepared using the synthetic procedures described in Example 26. Table 25. Additional compounds prepared according to Example 26.

TABLE 25
Additional compounds prepared according to Example 26.

| Compound | | | |
|---|---|---|---|
| # | Structure | IUPAC Name | LCMS |
| 111a | 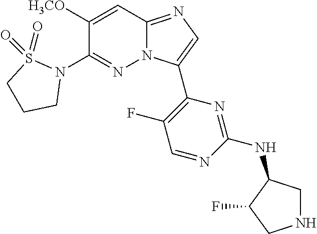 [SMILES: COc1cc2ncc(-c3nc(N[C@H]4CNC[C@@H]4F)ncc3F)n2nc1N1CCCS1(=O)=O] | 2-(3-(5-fluoro-2- (((3S,4S)-4- fluoropyrrolidin-3- yl)amino)pyrimidin-4- yl)-7- methoxyimidazo[1,2- b]pyridazin-6- yl)isothiazolidine 1,1- dioxide | 467.0 |
| 112a | 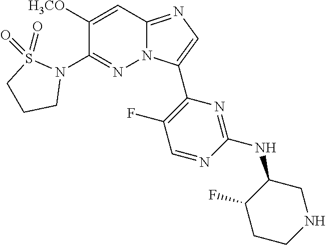 [SMILES: COc1cc2ncc(-c3nc(N[C@H]4CNCC[C@@H]4F)ncc3F)n2nc1N1CCCS1(=O)=O] | 2-(3-(5-fluoro-2- (((3S,4S)-4- fluoropiperidin-3- yl)amino)pyrimidin-4- yl)-7- methoxyimidazo[1,2- b]pyridazin-6- yl)isothiazolidine 1,1- dioxide | 481.1 |

### Example 27

### Exemplary Synthetic Procedure #27 (Compounds 117a-118a)

### Compound 117a, 5-fluoro-N-((3S,4S)-4-fluoropiperidin-3-yl)-4-(6-(oxetan-3-yl)imidazo[1,2-a]pyrazin-3-yl)pyrimidin-2-amine

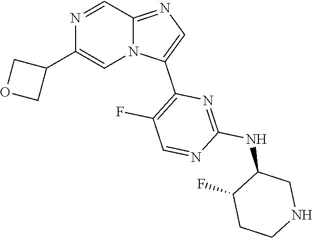 [SMILES: Fc1cnc(N[C@H]2CNCC[C@@H]2F)nc1-c1cnc2cnc(C3COC3)cn12]

### Step A. tert-butyl N-(5-bromopyrazin-2-yl)-N-tert-butoxycarbonyl-carbamate

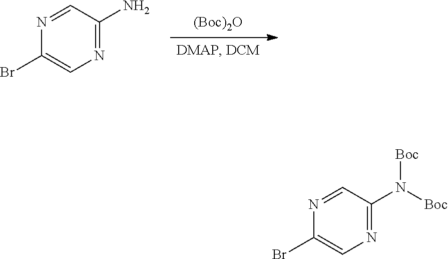

To a solution of 5-bromopyrazin-2-amine (2.00 g, 11.49 mmol) in dichloromethane (500 mL) were added N,N-dimethylpyridin-4-amine (0.140 g, 1.15 mmol) and tert-butoxycarbonyl tert-butyl carbonate (7.53 g, 34.5 mmol, 7.92 mL). The resulting reaction was stirred at room temperature for 2 hours. The reaction was then diluted with water (50 mL) and extracted with ethyl acetate (3×50 mL). The organic extracts were combined, washed with saturated aqueous sodium chloride solution (3×20 mL), dried over sodium sulfate, filtered, and concentrated under reduced pressure. The resulting crude product was purified by flash chromatography on silica gel (0-10% ethyl acetate in petroleum ether) to provide the title compound: LCMS m/z 219.8 [M+H]⁺; ¹H NMR (400 MHz, CD₃OD) δ 8.67 (d, J=1.3 Hz, 1H), 8.49 (d, J=1.4 Hz, 1H), 1.44 (s, 18H).

### Step B. tert-butyl (5-(oxetan-3-yl)pyrazin-2-yl)carbamate

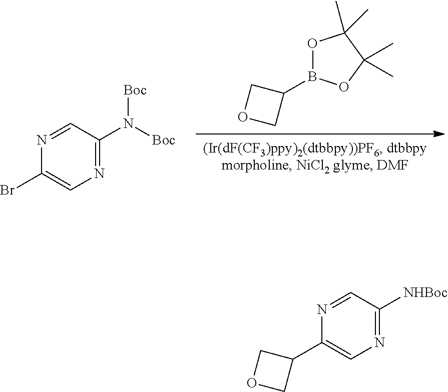 [SMILES: CC(C)(C)OC(=O)N(C(=O)OC(C)(C)C)c1cnc(Br)cn1 ; CC1(C)OB(C2COC2)OC1(C)C ; "(Ir(dF(CF3)ppy)2(dtbbpy))PF6, dtbbpymorpholine, NiCl2 glyme, DMF" ; CC(C)(C)OC(=O)Nc1cnc(C2COC2)cn1]

A mixture of tert-butyl N-(5-bromopyrazin-2-yl)-N-tert-butoxycarbonylcarbamate (0.500 g, 1.34 mmol), 4,4,5,5-tetramethyl-2-(oxetan-3-yl)-1,3,2-dioxaborolane (0.492 g, 2.67 mmol), dichloronickel 1,2-dimethoxyethane (0.015 g, 0.067 mmol), dtbbpy (0.018 g, 0.067 mmol), bis[3,5-difluoro-2-[5-(trifluoromethyl)-2-pyridyl]phenyl]iridium 4-tert-butyl-2-(4-tert-butyl-2-pyridyl)pyridine hexafluorophosphate (0.015 g, 0.014 mmol), and morpholine (0.0175 g, 2.00 mmol, 0.176 mL) in N,N-dimethylformamide (10 mL) was purged with argon. The reaction was then stirred at room temperature and irradiated with two 34 W blue LEDs (vials approximately 6 cm away from the light source) for 20 hours. The reaction was then diluted with water (100 mL) and extracted with ethyl acetate (3×100 mL). The organic extracts were combined, washed with saturated aqueous sodium chloride solution (3×100 mL), dried over sodium sulfate, filtered, and concentrated under reduced pressure. The resulting crude product was purified by HPLC (Phenomenex Luna C18 column, 3 micron, 250×100 mm; 25-55% acetonitrile in water containing 0.1% trifluoroacetic acid) to provide the title compound: LCMS m/z 352.15 [M+H]⁺; ¹HNMR (400 MHz, CDCl₃) δ 9.31 (s, 1H), 8.13 (d, J=1.3 Hz, 1H), 5.05 (dd, J=5.8, 8.4 Hz, 2H), 4.94 (t, J=6.3 Hz, 2H), 4.46-4.32 (m, 1H), 1.56 (s, 9H).

### Step C. 5-(oxetan-3-yl)pyrazin-2-amine

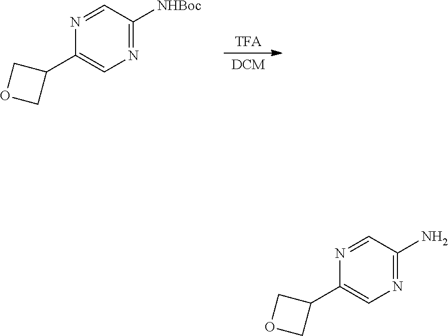

To a solution of tert-butyl N-[5-(oxetan-3-yl)pyrazin-2-yl]carbamate (0.500 g, 1.99 mmol) in dichloromethane (5 mL) was added trifluoroacetic acid (7.68 g, 67.3 mmol, 5.00 mL). The resulting reaction was stirred at room temperature for 1.5 hours. The reaction was then cooled to 0° C., basified to pH ~9 by addition of solid sodium bicarbonate, diluted with water (5 mL), and extracted with 30% isopropanol in chloroform (3×15 mL). The organic extracts were combined, washed with saturated aqueous sodium chloride solution (15 mL), dried over sodium sulfate, filtered, and concentrated under reduced pressure to provide the title compound: LCMS m/z 152.1 [M+H]⁺; ¹HNMR (400 MHz, CH₃OD) δ 8.02 (d, J=1.0 Hz, 1H), 7.79 (s, 1H), 5.00-4.95 (m, 2H), 4.87-4.84 (m, 2H), 4.36-4.27 (m, 1H).

### Step D. 3-(2-bromo-5-fluoropyrimidin-4-yl)-6-(oxetan-3-yl)imidazo[1,2-a]pyrazine

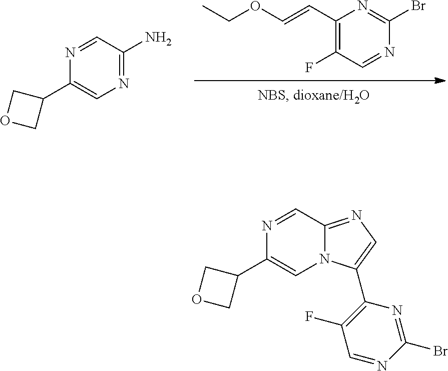

To a cooled 0° C. solution of 2-bromo-4-[(E)-2-ethoxyvinyl]-5-fluoro-pyrimidine (0.392 g, 1.59 mmol) in dioxane (4 mL) and water (1.3 mL) was added 1-bromopyrrolidine-2,5-dione (0.283 g, 1.59 mmol). The resulting reaction was stirred for 30 minutes while warming to room temperature. 5-(Oxetan-3-yl)pyrazin-2-amine (0.200 g, 1.32 mmol) and sodium bicarbonate (0.445 g, 5.29 mmol) were then added, and the reaction was heated at 80° C. for 4 hours. The reaction was then cooled to room temperature, diluted with water (10 mL), and extracted with ethyl acetate (3×10 mL). The organic extracts were combined, washed with saturated aqueous sodium chloride solution (10 mL), dried over sodium sulfate, filtered, and concentrated under reduced pressure. The resulting crude product was purified by flash chromatography on silica gel (0-70% methanol in petroleum ether) to provide the title compound: LCMS m/z 350.1 [M+H]⁺; ¹HNMR (400 MHz, DMSO-d₆) δ 9.47-9.41 (m, 2H), 8.93 (d, J=3.3 Hz, 1H), 8.62 (d, J=3.9 Hz, 1H), 5.02-4.94 (m, 2H), 4.85-4.81 (m, 2H), 4.59-4.47 (m, 1H).

### Step E. tert-butyl (3S,4S)-4-fluoro-3-((5-fluoro-4-(6-(oxetan-3-yl)imidazo[1,2-a]pyrazin-3-yl)pyrimidin-2-yl)amino)piperidine-1-carboxylate

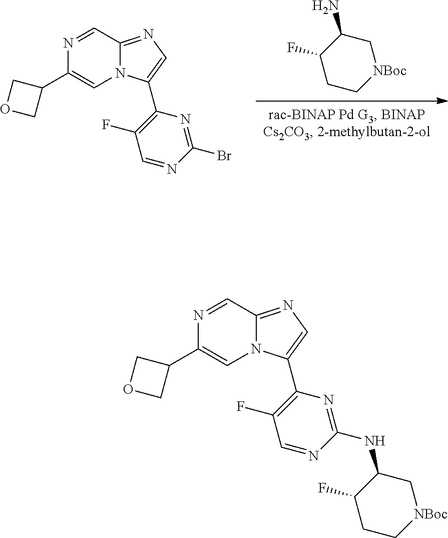

A mixture of 3-(2-bromo-5-fluoro-pyrimidin-4-yl)-6-(oxetan-3-yl)imidazo[1,2-a]pyrazine (0.050 g, 0.143 mmol), tert-butyl (3S,4S)-3-amino-4-fluoro-piperidine-1-carboxylate (0.047 g, 0.214 mmol), rac-(R)-(+)-2,2′-bis(diphenylphosphino)-1,1′-binaphthyl-Pd-G3 (0.014 g, 0.014 mmol), (R)-(+)-2,2′-bis(diphenylphosphino)-1,1′-binaphthyl (0.013 g, 0.021 mmol), and cesium carbonate (0.116 g, 0.357 mmol) in 2-methylbutan-2-ol (3 mL) was purged with nitrogen, and was then heated at 80° C. for 6 hours under nitrogen atmosphere. The reaction was cooled to room temperature, filtered, and concentrated under reduced pressure to provide the title compound: LCMS m/z 488.2 [M+H]⁺.

### Step F. 5-fluoro-N-((3S,4S)-4-fluoropiperidin-3-yl)-4-(6-(oxetan-3-yl)imidazo[1,2-a]pyrazin-3-yl)pyrimidin-2-amine

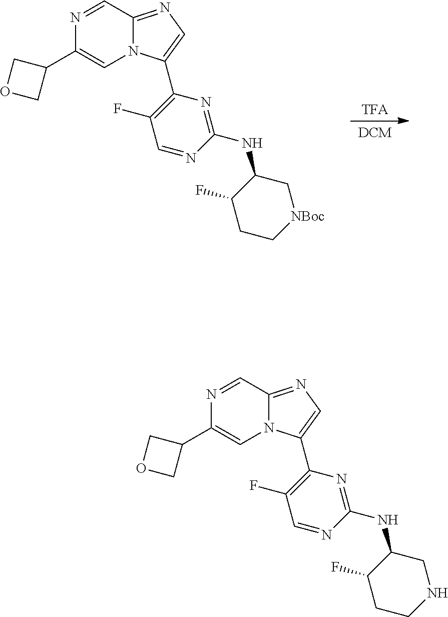

To a solution of tert-butyl (3S,4S)-4-fluoro-3-[[5-fluoro-4-[6-(oxetan-3-yl)imidazo[1,2-a]pyrazin-3-yl]pyrimidin-2-yl]amino]piperidine-1-carboxylate (0.100 g, 0.205 mmol) in dichloromethane (2 mL) was added trifluoroacetic acid (0.50 mL). The resulting reaction was stirred at room temperature for 1.5 hours. The reaction was then cooled to 0° C., basified to pH ~9 by addition of solid sodium bicarbonate, diluted with water (5 mL), and extracted with 30% isopropanol in chloroform (3×5 mL). The organic extracts were combined, dried over sodium sulfate, filtered, and concentrated under reduced pressure. The resulting crude product was purified by HPLC (Waters Xbridge BEH C18 column, 10 micron, 100×30 mm; 1-30% acetonitrile in water containing 0.1% ammonium bicarbonate) to provide the title compound: LCMS m/z 388.2 [M+H]⁺; ¹HNMR (400 MHz, CD₃OD) δ 9.87 (s, 1H), 9.26 (s, 1H), 8.47 (d, J=3.6 Hz, 1H), 8.35 (d, J=3.5 Hz, 1H), 5.13-5.02 (m, 4H), 4.73-4.57 (m, 2H), 4.14-4.10 (m, 1H), 3.29-3.19 (m, 1H), 3.15-3.06 (m, 1H), 2.74-2.64 (m, 1H), 2.64-2.53 (m, 1H), 2.28-2.17 (m, 1H), 1.86-1.73 (m, 1H).

The compound in Table 26 were all prepared using the synthetic procedures described in Example 27.

TABLE 26
Additional compounds prepared according to Example 27.

| Compound | | | |
|---|---|---|---|
| # | Structure | IUPAC Name | LCMS |
| 118a | 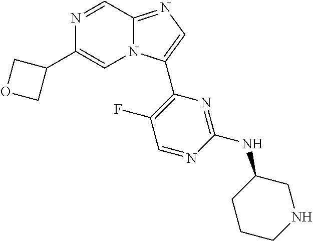 [SMILES: Fc1cnc(N[C@@H]2CCCNC2)nc1-c1cnc2cnc(C3COC3)cn12] | (R)-5-fluoro-4-(6- (oxetan-3- yl)imidazo[1,2- a]pyrazin-3-yl)-N- (piperidin-3- yl)pyrimidin-2-amine | 370.2 |

### Example 28

### Exemplary Synthetic Procedure #28 (Compounds 119a-121aa)

### Compound 119a, 5-fluoro-N-((3S,4S)-4-fluoropyrrolidin-3-yl)-4-(7-methoxy-6-morpholinoimidazo[1,2-b]pyridazin-3-yl)pyrimidin-2-amine

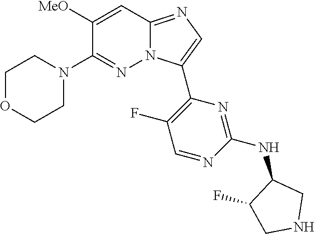 [SMILES: COc1cc2ncc(-c3nc(N[C@H]4CNC[C@@H]4F)ncc3F)n2nc1N1CCOCC1]

### Step A. 4-(6-chloro-4-methoxypyridazin-3-yl)morpholine

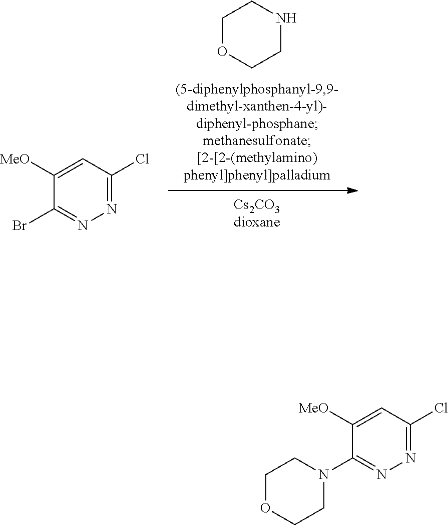

A mixture of 3-bromo-6-chloro-4-methoxypyridazine (5.00 g, 22.4 mmol), morpholine (2.34 g, 26.9 mmol, 2.36 mL), (5-diphenylphosphanyl-9,9-dim ethyl-xanthen-4-yl)-diphenyl-phosphanemethanesulfonate[2-[2-(methylamino)phenyl]phenyl]palladium (2.15 g, 2.24 mmol), and cesium carbonate (14.58 g, 44.75 mmol) in dioxane (80 mL) was purged with nitrogen, and was then heated at 90° C. for 16 hours under nitrogen atmosphere. The reaction was then cooled to room temperature, diluted with water (80 mL), and extracted with ethyl acetate (3×80 mL). The organic extracts were combined, washed with saturated aqueous sodium chloride solution (80 mL), dried over sodium sulfate, filtered, and concentrated under reduced pressure. The resulting crude product was purified by flash chromatography on silica gel (0-50% ethyl acetate in petroleum ether) to provide the title compound: LCMS m/z 229.9 [M+H]⁺; ¹H NMR (400 MHz, CD₃OD) δ 7.16 (s, 1H), 4.01 (s, 3H), 3.84-3.78 (m, 4H), 3.52-3.41 (m, 4H).

### Step B. tert-butyl (5-methoxy-6-morpholinopyridazin-3-yl)carbamate

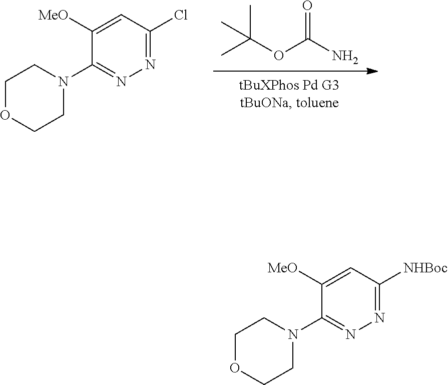

A mixture of 4-(6-chloro-4-methoxypyridazin-3-yl)morpholine (0.400 g, 1.74 mmol), tert-butyl carbamate (0.408.06 g, 3.48 mmol), [2-(2-aminophenyl)phenyl]-methylsulfonyloxypalladium ditert-butyl-[2-(2,4,6-triisopropylphenyl)phenyl]phosphane (0.138. g, 0.174 mmol), and a solution of sodium tert-butoxide (2 M in THF, 1.74 mL) in toluene (30 mL) was purged with nitrogen, and was then heated at 90° C. for 16 hours under nitrogen atmosphere. The reaction was then cooled to room temperature, diluted with water (100 mL), and extracted with ethyl acetate (3×100 mL). The organic extracts were combined, washed with saturated aqueous sodium chloride solution (100 mL), dried over sodium sulfate, filtered, and concentrated under reduced pressure. The resulting crude product was purified by flash chromatography on silica gel (0-30% ethyl acetate in petroleum ether) to provide the title compound: LCMS m/z 311.0 [M+H]⁺; ¹H NMR (400 MHz, CD30D) b 7.69 (s, 1H), 3.94 (s, 3H), 3.86-3.77 (m, 4H), 3.40-3.33 (m, 4H), 1.54 (s, 9H).

### Step C. 5-methoxy-6-morpholinopyridazin-3-amine

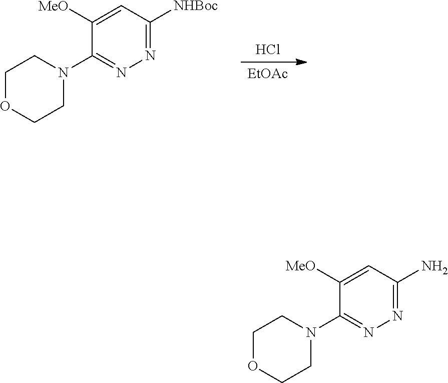 [SMILES: "EtOAc" ; COc1cc(NC(=O)OC(C)(C)C)nnc1N1CCOCC1 ; Cl ; COc1cc(N)nnc1N1CCOCC1]

A solution of tert-butyl (5-methoxy-6-morpholinopyridazin-3-yl)carbamate (0.580 g, 1.87 mmol) in hydrochloric acid/ethyl acetate (120 mL) was stirred for 2 hours at room temperature. The reaction was then concentrated under reduced pressure to provide the title compound: LCMS m/z 211.0 [M+H]⁺; ¹H NMR (400 MHz, CD₃OD) δ 6.65 (s, 1H), 4.05 (s, 3H), 3.83-3.75 (m, 4H), 3.34-3.31 (m, 4H).

### Step D. 4-(3-(2-bromo-5-fluoropyrimidin-4-yl)-7-methoxyimidazo[1,2-b]pyridazin-6-yl)morpholine

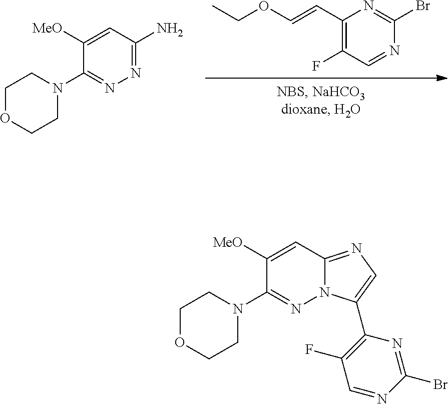

To a cooled 0° C. solution of (E)-2-bromo-4-(2-ethoxyvinyl)-5-fluoropyrimidine (1.06 g, 4.28 mmol) in dioxane (9 mL) and water (3 mL) was added 1-bromopyrrolidine-2,5-dione (0.762 g, 4.28 mmol). The reaction was then stirred for 30 minutes while warming to room temperature. 5-Methoxy-6-morpholinopyridazin-3-amine (0.450 g, 2.14 mmol) and sodium bicarbonate (0.360 g, 4.28 mmol) were added, and the reaction was then heated at 80° C. for 16 hours. The reaction was then cooled to room temperature, diluted with water (10 mL), and extracted with ethyl acetate (3×10 mL). The organic extracts were combined, washed with saturated aqueous sodium chloride solution (10 mL), dried over sodium sulfate, filtered, and concentrated under reduced pressure. The resulting crude product was purified by flash chromatography on silica gel (0-100% ethyl acetate in petroleum ether) to provide the title compound: LCMS m/z 409.1 [M+H]⁺.

### Step E. tert-butyl (3S,4S)-3-fluoro-4-((5-fluoro-4-(7-methoxy-6-morpholinoimidazo[1,2-b]pyridazin-3-yl)pyrimidin-2-yl)amino)pyrrolidine-1-carboxylate

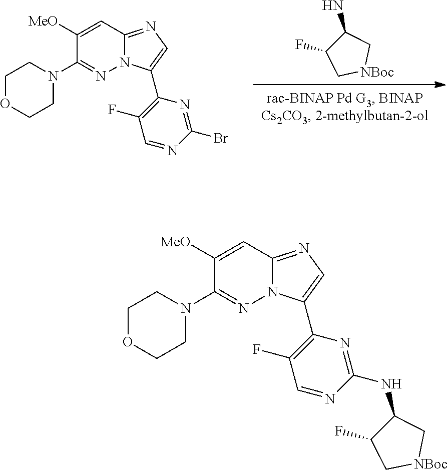

A mixture of 4-(3-(2-bromo-5-fluoropyrimidin-4-yl)-7-methoxyimidazo[1,2-b]pyridazin-6-yl)morpholine (0.080 g, 0.196 mmol), tert-butyl (3S,4S)-3-amino-4-fluoropyrrolidine-1-carboxylate (0.060 g, 0.293 mmol), methanesulfonato[2,2-bis(diphenylphosphino)-1,1-binaphthyl](2-amino-1,1-biphenyl-2-yl)palladium(II) (0.019 g, 0.019 mmol), (R)-(+)-2,2′-bis(diphenylphosphino)-1,1′-binaphthyl (0.012 g, 0.020 mmol), and cesium carbonate (0.191 g, 0.586 mmol) in 2-methylbutan-2-ol (3 mL) was purged with nitrogen, and was then heated at 80° C. for 16 hours under nitrogen atmosphere. The reaction was then cooled to room temperature and concentrated under reduced pressure to provide the title compound: LCMS m/z 533.3 [M+H]⁺.

### Step F. 5-fluoro-N-((3S,4S)-4-fluoropyrrolidin-3-yl)-4-(7-methoxy-6-morpHolinoimidazo[1,2-b]pyridazin-3-yl)pyrimidin-2-amine

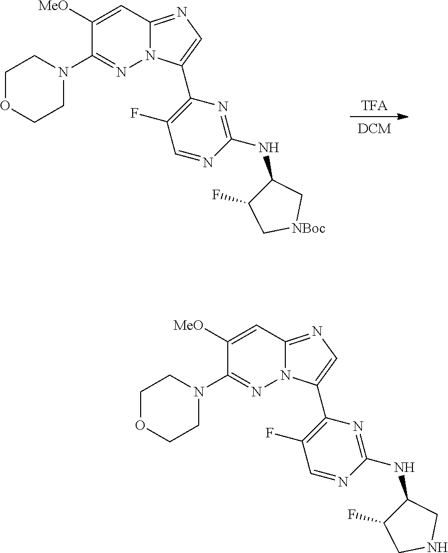

To a solution of tert-butyl (3S,4S)-3-fluoro-4-((5-fluoro-4-(7-methoxy-6-morpholinoimidazo[1,2-b]pyridazin-3-yl)pyrimidin-2-yl)amino)pyrrolidine-1-carboxylate (0.120 g, 0.225 mmol) in dichloromethane (3 mL) was added trifluoroacetic acid (0.80 mL). The resulting reaction was stirred at room temperature for 2 hours, and was then concentrated under reduced pressure. The resulting crude product was purified by HPLC (Phenomenex Luna C18 column, 5 micron, 150×40 mm; 5-35% acetonitrile in water containing 0.04% ammonium bicarbonate) to provide the title compound: LCMS m/z 433.1 [M+H]⁺; ¹H NMR (400 MHz, CD3OD) δ 8.33 (d, J=3.0 Hz, 1H), 7.92 (d, J=1.6 Hz, 1H), 7.29 (s, 1H), 5.22-4.99 (d, J=50.4 Hz, 1H), 4.55 (td, J=5.0, 19.1 Hz, 1H), 4.02 (s, 3H), 3.87-3.78 (m, 4H), 3.52-3.36 (m, 5H), 3.23-3.07 (m, 2H), 2.87 (dd, J=3.9, 12.3 Hz, 1H).

The compounds in Table 27 were all prepared using the synthetic procedures described in Example 28.

TABLE 27
Additional compounds prepared according to Example 28.

| Compound | | | |
|---|---|---|---|
| # | Structure | IUPAC Name | LCMS |
| 120a | 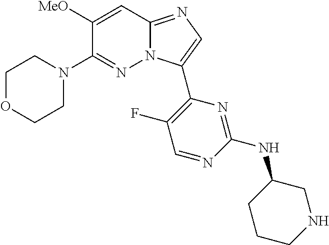 [SMILES: COc1cc2ncc(-c3nc(N[C@@H]4CCCNC4)ncc3F)n2nc1N1CCOCC1] | (R)-5-fluoro-4-(7- methoxy-6- morpholinoimidazo[1,2- b]pyridazin-3-yl)-N- (piperidin-3- yl)pyrimidin-2-amine | 429.2 |
| 121a | 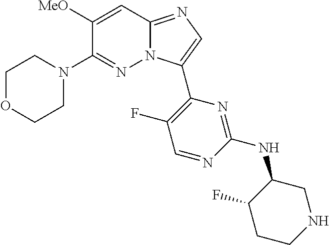 [SMILES: COc1cc2ncc(-c3nc(N[C@H]4CNCC[C@@H]4F)ncc3F)n2nc1N1CCOCC1] | 5-fluoro-N-((3S,4S)-4- fluoropiperidin-3-yl)-4- (7-methoxy-6- morpholinoimidazo[1,2- b]pyridazin-3- yl)pyrimidin-2-amine | 447.2 |

### Example 29

### Exemplary Synthetic Procedure #29 (Compounds 122a-124a)

### Compound 122a, 4-(3-(5-fluoro-2-(((3S,4S)-4-fluoropiperidin-3-yl)amino)pyrimidin-4-yl)-7-methoxyimidazo[1,2-b]pyridazin-6-yl)thiomorpholine 1,1-dioxide

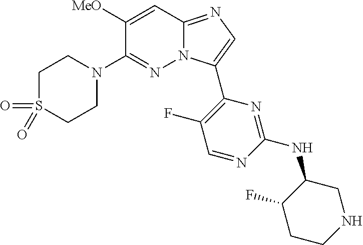 [SMILES: COc1cc2ncc(-c3nc(N[C@H]4CNCC[C@@H]4F)ncc3F)n2nc1N1CCS(=O)(=O)CC1]

### Step A. 4-(6-chloro-4-methoxypyridazin-3-yl)thiomorpholine 1,1-dioxide

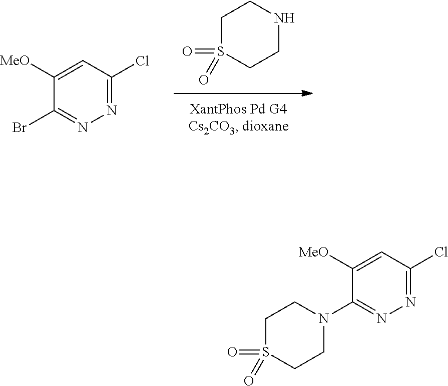 [SMILES: COc1cc(Cl)nnc1Br ; O=S1(=O)CCNCC1 ; COc1cc(Cl)nnc1N1CCS(=O)(=O)CC1]

A mixture of 3-bromo-6-chloro-4-methoxy-pyridazine (5.00 g, 22.4 mmol), 1,4-thiazinane 1,1-dioxide (4.54 g, 33.6 mmol), (5-diphenylphosphanyl-9,9-dimethyl-xanthen-4-yl)-diphenyl-phosphane methanesulfonate[2-[2-(methylamino)phenyl]phenyl]palladium (1.08 g, 1.12 mmol), and cesium carbonate (14.58 g, 44.75 mmol) in dioxane (100 mL) was purged with nitrogen, and was then heated at 90° C. for 16 hours under nitrogen atmosphere. The reaction was then cooled to room temperature, diluted with water (100 mL), and extracted with ethyl acetate (3×100 mL). The organic extracts were combined, washed with saturated aqueous sodium chloride solution (100 mL), dried over sodium sulfate, filtered, and concentrated under reduced pressure. The resulting crude product was triturated with methanol (3×120 mL) to provide the title compound: LCMS m/z 277.9 [M+H]⁺¹; HNMR (400 MHz, DMSO-d₆) δ 7.34 (s, 1H), 3.97-3.90 (m, 8H), 3.29-3.21 (m, 3H).

### Step B. tert-butyl (6-(1,1-dioxidothiomorpholino)-5-methoxypyridazin-3-yl)carbamate

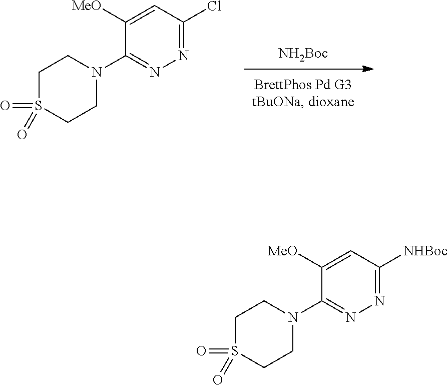 [SMILES: COc1cc(Cl)nnc1N1CCS(=O)(=O)CC1 ; COc1cc(NC(=O)OC(C)(C)C)nnc1N1CCS(=O)(=O)CC1 ; "NH2Boc"]

A mixture of tert-butyl carbamate (2.89 g, 24.7 mmol), 4-(6-chloro-4-methoxy-pyridazin-3-yl)-1,4-thiazinane-1,1-dioxide (2.74 g, 9.87 mmol), sodium tert-butoxide solution (2 M, 9.87 mL), and [2-(2-aminophenyl)phenyl]-methylsulfonyloxy-palladium dicyclohexyl-[3,6-dimethoxy-2-(2,4,6triisopropylphenyl)phenyl]phosphane (0.894 g, 0.987 mmol) in dioxane (50 mL) was purged with nitrogen, and was then heated at 100° C. for 4 hours under nitrogen atmosphere. The reaction was then cooled to room temperature, diluted with water (50 mL), and extracted with ethyl acetate (3×50 mL). The organic extracts were combined, washed with saturated aqueous sodium chloride solution (50 mL), dried over sodium sulfate, filtered, and concentrated under reduced pressure. The resulting crude product was purified by flash chromatography on silica gel (0-60% ethyl acetate in petroleum ether) to provide the title compound: LCMS m/z 359.1 [M+H]⁺; ¹HNMR (400 MHz, CD₃OD) δ 7.73 (s, 1H), 3.97 (s, 3H), 3.97-3.92 (m, 4H), 3.25-3.20 (m, 4H), 1.54 (s, 9H).

### Step C. 4-(6-amino-4-methoxypyridazin-3-yl)thiomorpholine 1,1-dioxide

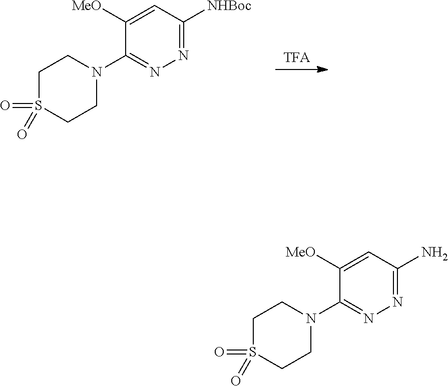 [SMILES: COc1cc(NC(=O)OC(C)(C)C)nnc1N1CCS(=O)(=O)CC1 ; COc1cc(N)nnc1N1CCS(=O)(=O)CC1 ; "TFA"]

To a solution of tert-butyl N-[6-(1,1-dioxo-1,4-thiazinan-4-yl)-5-methoxy-pyridazin-3-yl]carbamate (1.24 g, 3.46 mmol) in dichloromethane (10 mL) was added trifluoroacetic acid (9.21 g, 80.78 mmol, 6.00 mL). The resulting reaction was stirred at room temperature for 1.5 hours, and was then concentrated under reduced pressure to provide the title compound: LCMS m/z 259.2 [M+H]⁺; ¹HNMR (400 MHz, CD₃OD) δ 6.42 (s, 1H), 3.90 (s, 3H), 3.84-3.78 (m, 4H), 3.25-3.16 (m, 4H).

### Step D. 4-(3-(2-bromo-5-fluoropyrimidin-4-yl)-7-methoxyimidazo[1,2-b]pyridazin-6-yl)thiomorpholine 1,1-dioxide

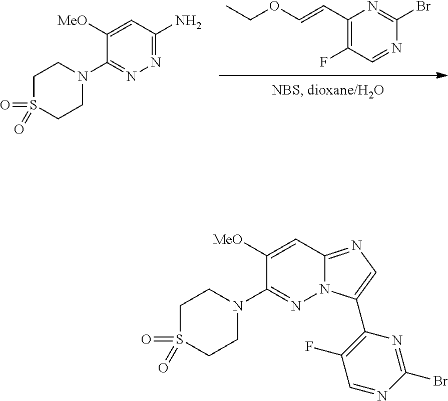

To a cooled 0° C. solution of 2-bromo-4-[(E)-2-ethoxyvinyl]-5-fluoro-pyrimidine (0.230 g, 0.929 mmol) in dioxane (4 mL) and water (1.3 mL) was added 1-bromopyrrolidine-2,5-dione (0.165 g, 0.929 mmol). The resulting reaction was then stirred for 30 minutes while warming to room temperature. 6-(1,1-Dioxo-1,4-thiazinan-4-yl)-5-methoxy-pyridazin-3-amine (0.100 g, 0.387 mmol) and sodium bicarbonate (0.065 g, 0.774 mmol) were added, and the reaction was then heated at 80° C. for 2 hours. The reaction was then cooled to room temperature, diluted with water (15 mL), and extracted with ethyl acetate (3×45 mL). The organic extracts were combined, washed with saturated aqueous sodium chloride solution (15 mL), dried over sodium sulfate, filtered, and concentrated under reduced pressure. The resulting crude product was purified by flash chromatography on silica gel (0-3% methanol in petroleum ether) to provide the title compound: LCMS m/z 457.1 [M+H]⁺; ¹H NMR (400 MHz, DMSO-d₆) δ 8.81 (d, J=3.0 Hz, 1H), 8.26 (d, J=3.6 Hz, 1H), 7.66 (s, 1H), 4.02-4.01 (m, 4H), 4.00 (s, 3H), 3.39-3.38 (m, 4H).

### Step E. tert-butyl (3S,4S)-3-((4-(6-(1,1-dioxidothiomorpholino)-7-methoxyimidazo[1,2-b]pyridazin-3-yl)-5-fluoropyrimidin-2-yl)amino)-4-fluoropiperidine-1-carboxylate

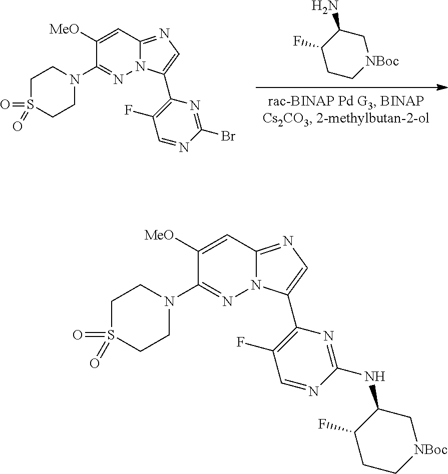

A mixture of 4-[3-(2-bromo-5-fluoro-pyrimidin-4-yl)-7-methoxy-imidazo[1,2-b]pyridazin-6-yl]-1,4-thiazinane 1,1-dioxide (0.050 g, 0.109 mmol), tert-butyl (3S,4S)-3-amino-4-fluoro-piperidine-1-carboxylate (0.036 g, 0.164 mmol), methanesulfonato[2,2-bis(diphenylphosphino)-1,1-binaphthyl](2-amino-1,1-biphenyl-2-yl)palladium(II) (0.011 g, 0.011 mmol), (R)-(+)-2,2′-bis(diphenylphosphino)-1,1′-binaphthyl (0.011 g, 0.016 mmol), and cesium carbonate (0.089 g, 0.273 mmol) in 2-methylbutan-2-ol (3.5 mL) was purged with nitrogen, and was then heated at 80° C. for 6 hours under nitrogen atmosphere. The reaction was then cooled to room temperature, filtered, and concentrated under reduced pressure to provide the title compound: LCMS m/z 595.2 [M+H]⁺.

### Step F. 4-(3-(5-fluoro-2-(((3S,4S)-4-fluoropiperidin-3-yl)amino)pyrimidin-4-yl)-7-methoxyimidazo[1,2-b]pyridazin-6-yl)thiomorpholine 1,1-dioxide

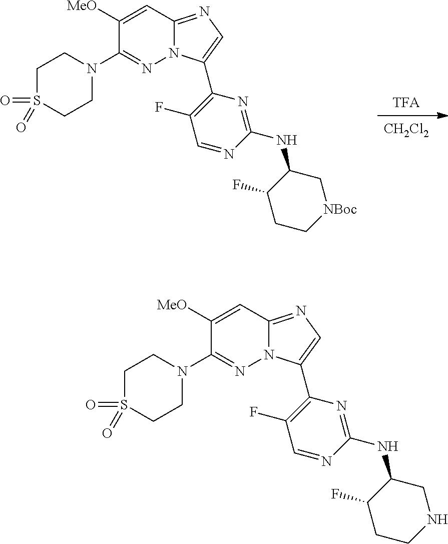 [SMILES: COc1cc2ncc(-c3nc(N[C@H]4CN(C(=O)OC(C)(C)C)CC[C@@H]4F)ncc3F)n2nc1N1CCS(=O)(=O)CC1 ; "CH2Cl2" ; COc1cc2ncc(-c3nc(N[C@H]4CNCC[C@@H]4F)ncc3F)n2nc1N1CCS(=O)(=O)CC1 ; "TFA"]

To a solution of tert-butyl (3S,4S)-3-[[4-[6-(1,1-dioxo-1,4-thiazinan-4-yl)-7-methoxy-imidazo[1,2-b]pyridazin-3-yl]-5-fluoro-pyrimidin-2-yl]amino]-4-fluoro-piperidine-1-carboxylate (0.050 g, 0.084 mmol) in dichloromethane (3 mL) was added trifluoroacetic acid (1.54 g, 13.5 mmol, 1.00 mL). The resulting reaction was stirred at room temperature for 1.5 hours, and was then concentrated under reduced pressure. The crude product thus obtained was purified by HPLC (Waters Xbridge Prep OBD C18 column, 10 micron, 150×40 mm; 15-55% acetonitrile in water containing 0.1% ammonium bicarbonate) to provide the title compound: LCMS m/z 495.0 [M+H]⁺; ¹H NMR (400 MHz, CD₃OD) δ 8.32 (d, J=2.8 Hz, 1H), 7.96 (d, J=2.1 Hz, 1H), 7.36 (s, 1H), 4.72-4.51 (m, 1H), 4.18-4.10 (m, 1H), 4.05-4.01 (m, 7H), 3.38-3.37 (m, 4H), 3.29-3.24 (m, 1H), 3.10-3.01 (m, 1H), 2.65 (t, J=10.8 Hz, 1H), 2.50 (dd, J=9.4, 12.7 Hz, 1H), 2.16 (d, J=3.5 Hz, 1H), 1.81-1.67 (m, 1H).

The compounds in Table 28 were all prepared using the synthetic procedures described in Example 29.

TABLE 28
Additional compounds prepared according to Example 29.

| Compound | | | |
|---|---|---|---|
| # | Structure | IUPAC Name | LCMS |
| 123a | 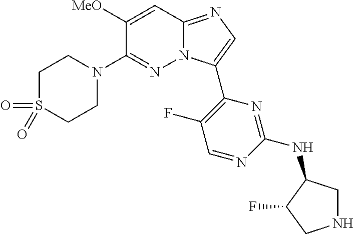 [SMILES: COc1cc2ncc(-c3nc(N[C@H]4CNC[C@@H]4F)ncc3F)n2nc1N1CCS(=O)(=O)CC1] | 4-(3-(5-fluoro-2- (((3S,4S)-4- fluoropyrrolidin-3- yl)amino)pyrimidin-4- yl)-7- methoxyimidazo[1,2- b]pyridazin-6- yl)thiomorpholine 1,1- dioxide | 481.0 |
| 124a | 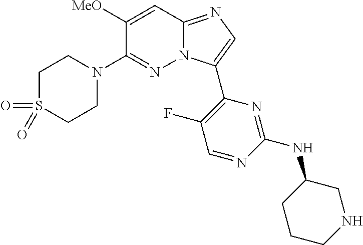 [SMILES: COc1cc2ncc(-c3nc(N[C@@H]4CCCNC4)ncc3F)n2nc1N1CCS(=O)(=O)CC1] | (R)-4-(3-(5-fluoro-2- (piperidin-3- ylamino)pyrimidin-4-yl)- 7-methoxyimidazo[1,2- b]pyridazin-6- yl)thiomorpholine 1,1- dioxide | 477.0 |

### Example 30

### Exemplary Synthetic Procedure #30 (Compounds 125a-127a)

### Compounds 125a & 126a, trans & cis-3-(3-(5-fluoro-2-(((3S,4S)-4-fluoropiperidin-3-yl)amino)pyrimidin-4-yl)-7-methoxyimidazo[1,2-b]pyridazin-6-yl)cyclobutan-1-ol

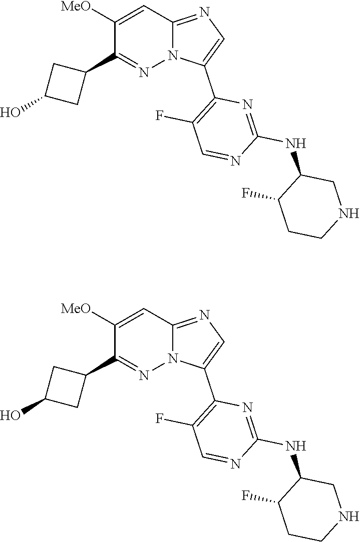 [SMILES: COc1cc2ncc(-c3nc(N[C@H]4CNCC[C@@H]4F)ncc3F)n2nc1[C@H]1C[C@H](O)C1 ; COc1cc2ncc(-c3nc(N[C@H]4CNCC[C@@H]4F)ncc3F)n2nc1[C@H]1C[C@@H](O)C1]

### Step A. 3-(methoxycarbonyl)cyclobutyl benzoate

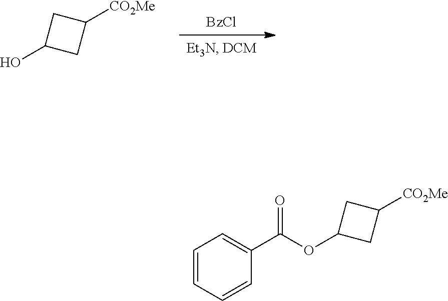

A mixture of methyl 3-hydroxycyclobutanecarboxylate (20.00 g, 153.7 mmol) and triethylamine (18.66 g, 184.4 mmol, 25.67 mL) in dichloromethane (500 mL) was purged with nitrogen and cooled to 0° C. Benzoyl chloride (25.92 g, 184.4 mmol, 21.41 mL) was added, and the resulting reaction was stirred at 0° C. for 3 hours. The reaction was then diluted with water (1000 mL) and extracted with dichloromethane (3×1000 mL). The organic extracts were combined, washed with saturated aqueous sodium chloride solution (3×600 mL), dried over anhydrous sodium sulfate, filtered, and concentrated under reduced pressure. The resulting crude product was purified by flash chromatography on silica gel (0-100% ethyl acetate in petroleum ether) to provide the title compound: LCMS m/z 235.2 [M+H]⁺; ¹H NMR (400 MHz, CD30D) 6 8.07-7.95 (m, 2H), 7.63-7.56 (m, 1H), 7.51-7.43 (m, 2H), 5.17-5.13 (m, 1H), 3.73-3.65 (s, 3H), 3.01-2.81 (m, 1H), 2.79-2.67 (m, 2H), 2.57-2.33 (m, 2H).

### Step B. 3-(benzoyloxy) cyclobutane-1-carboxylic acid

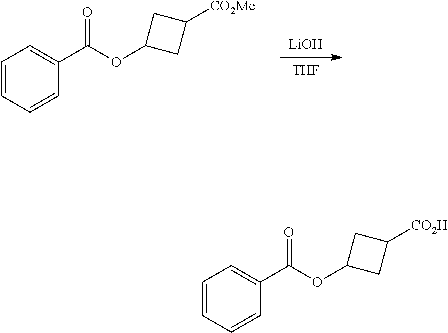 [SMILES: COC(=O)C1CC(OC(=O)c2ccccc2)C1 ; "LiOH" ; O=C(OC1CC(C(=O)O)C1)c1ccccc1 ; "THF"]

To a solution of (3-methoxycarbonylcyclobutyl)benzoate (56.00 g, 239.1 mmol) in tetrahydrofuran (1000 mL) was added aqueous lithium hydroxide solution (2 M, 144.0 mL). The resulting reaction was stirred at room temperature for 4 hours. The reaction was then concentrated under reduced pressure to remove tetrahydrofuran, acidified to pH ~5 by addition of hydrochloric acid (2 M, 80 mL), and filtered. The collected solids were dried under reduced pressure to provide the title compound: LCMS m/z 221.2 [M+H]⁺; ¹H NMR (400 MHz, CD3OD) δ 8.02-7.98 (m, 2H), 7.62-7.55 (m, 1H), 7.50-7.45 (m, 2H), 5.21-5.04 (m, 1H), 2.94-2.80 (m, 1H), 2.79-2.69 (m, 2H), 2.58-2.33 (m, 2H).

### Step C. 3-(6-chloro-4-methoxypyridazin-3-yl)cyclobutyl benzoate

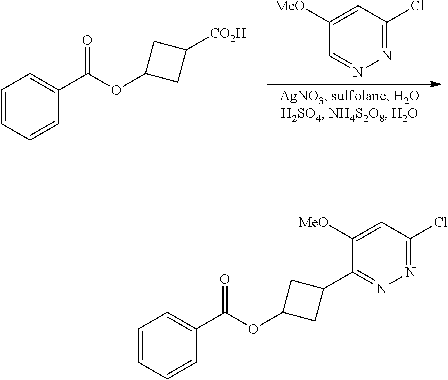

A mixture of 3-chloro-5-methoxy-pyridazine (40.00 g, 276.7 mmol), benzoyloxycyclobutanecarboxylic acid (67.03 g, 304.4 mmol), silver nitrate (23.50 g, 138.4 mmol), ammonia sulfoxy hydrogen sulfate (252.58 g, 1.11 mol, 240.55 mL), and sulfuric acid (27.14 g, 276.7 mmol, 14.75 mL) in water (400 mL) and sulfolane (400 mL) was heated at 80° C. for 2 hours. The reaction was then cooled to room temperature and stirred for 14 hours. The reaction was then neutralized to pH ~7 by addition of aqueous sodium hydroxide solution (30% w/w, 100 mL), diluted with water (100 mL), and extracted with ethyl acetate (3×300 mL). The organic extracts were combined, washed with saturated aqueous sodium chloride solution (2×300 mL), dried over anhydrous sodium sulfate, filtered, and concentrated under reduced pressure. The resulting crude product was purified by flash chromatography on silica gel (0-100% ethyl acetate in petroleum ether) to provide the title compound: LCMS m/z 319.0 [M+H]⁺. ¹H NMR (400 MHz, CD₃OD) δ 8.00 (d, J=7.1 Hz, 2H), 7.61-7.58 (m, 1H), 7.49-7.45 (m, 3H), 5.20-5.08 (m, 1H), 3.98 (s, 3H), 2.95 (m, 1H), 2.88-2.81 (m, 1H), 2.77-2.72 (m, 2H), 2.66-2.59 (m, 1H).

### Step D. 3-(6-chloro-4-methoxypyridazin-3-yl)cyclobutan-1-ol

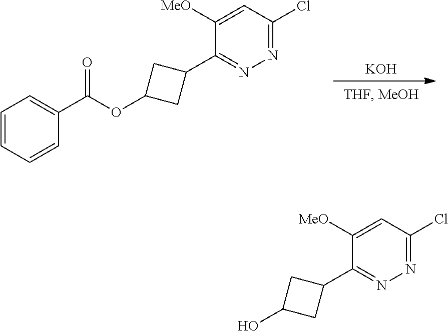

To a solution of [3-(6-chloro-4-methoxy-pyridazin-3-yl)cyclobutyl]benzoate (41.00 g, 128.6 mmol) in methanol (500 mL) and tetrahydrofuran (100 mL) was added potassium hydroxide (14.43 g, 2573 mmol). The resulting reaction was stirred at room temperature for 2 hours, and was then diluted with water (500 mL) and extracted with ethyl acetate (3×500 mL). The organic extracts were combined, washed with saturated aqueous sodium chloride solution (3×500 mL), dried over anhydrous sodium sulfate, filtered, and concentrated under reduced pressure. The resulting crude product was purified by flash chromatography on silica gel (0-100% ethyl acetate in petroleum ether) to provide the title compound: LCMS m/z 215.1 [M+H]⁺; ¹H NMR (400 MHz, CD₃OD) δ 7.27 (s, 1H), 4.35-4.22 (m, 1H), 3.91 (s, 1H), 3.39-3.31 (m, 1H), 2.72-2.65 (m, 2H), 2.42-2.17 (m, 2H).

### Step E. 3-(3-(benzyloxy)cyclobutyl)-6-chloro-4-methoxypyridazine

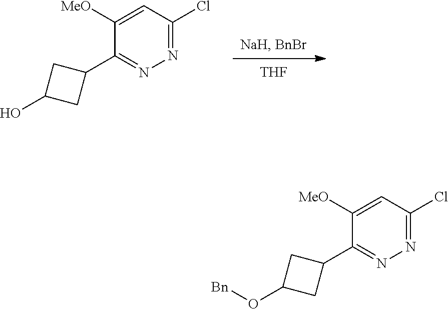

To a cooled 0° C. solution of 3-(6-chloro-4-methoxy-pyridazin-3-yl)cyclobutanol (12.00 g, 55.91 mmol) in THF (150 mL) was added in portions sodium hydride (3.35 g, 83.9 mmol, 60% purity). The resulting reaction mixture was stirred at 0° C. for 1 hour. Bromomethylbenzene (14.3 g, 83.9 mmol, 9.96 mL) was then added, and the reaction was stirred for 3 hours while warming to room temperature. The reaction was then cooled to 0° C., quenched by addition of water (50 mL), and extracted with ethyl acetate (3×50 mL). The organic extracts were combined, washed with saturated aqueous sodium chloride solution (2×50 mL), dried over anhydrous sodium sulfate, filtered, and concentrated under reduced pressure. The resulting crude product was purified by flash chromatography on silica gel (0-100% ethyl acetate in petroleum ether) to provide the title compound: LCMS m/z 305.1 [M+H]⁺; ¹H NMR (400 MHz, CD30D) 6 7.37-7.26 (m, 6H), 4.47 (s, 2H), 4.22-4.10 (m, 1H), 3.96 (s, 3H), 3.44-3.34 (m, 1H), 2.71-2.61 (m, 2H), 2.51-2.29 (m, 2H).

### Step F. tert-butyl (6-(3-(benzyloxy)cyclobutyl)-5-methoxypyridazin-3-yl)carbamate

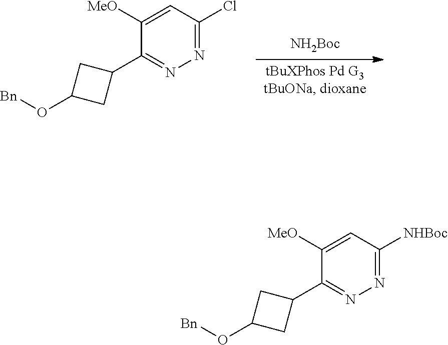 [SMILES: COc1cc(Cl)nnc1C1CC(OCc2ccccc2)C1 ; "tBuXPhos Pd G3tBuONa, dioxane" ; COc1cc(NC(=O)OC(C)(C)C)nnc1C1CC(OCc2ccccc2)C1 ; "NH2Boc"]

A mixture of 3-(3-benzyloxycyclobutyl)-6-chloro-4-methoxy-pyridazine (8.50 g, 27.9 mmol), tert-butyl carbamate (16.34 g, 139.5 mmol), sodium tert-butoxide solution (2 M in THF, 27.9 mL), and [2-(2-aminophenyl)phenyl]-methylsulfonyloxypalladium ditert-butyl-[2-(2,4,6-triisopropylphenyl)phenyl]phosphane (2.22 g, 2.79 mmol) in dioxane (100 mL) was purged with nitrogen, was then heated at 80° C. for 16 hours under nitrogen atmosphere. The reaction was then cooled to room temperature, diluted with water (100 mL), and extracted with ethyl acetate (3×200 mL). The organic extracts were combined, washed with saturated aqueous sodium chloride solution (3×100 mL), dried over anhydrous sodium sulfate, filtered, and concentrated under reduced pressure. The resulting crude product was purified by flash chromatography on silica gel (0-100% ethyl acetate in petroleum ether) to provide the title compound: LCMS m/z 386.1 [M+H]⁺; ¹H NMR (400 MHz, CD₃OD) δ 7.46-7.17 (m, 6H), 4.47 (s, 2H), 4.22-4.08 (m, 1H), 3.93 (s, 3H), 3.44-3.33 (m, 1H), 2.71-2.59 (m, 2H), 2.50-2.26 (m, 2H), 1.55 (s, 9H).

### Step G. 6-(3-(benzyloxy)cyclobutyl)-5-methoxypyridazin-3-amine

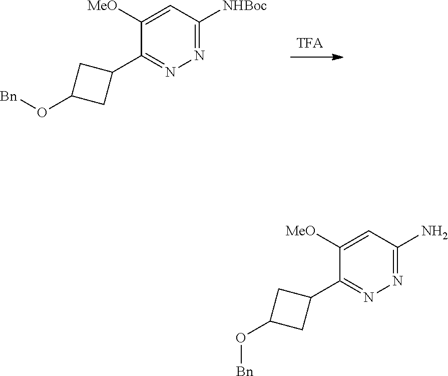 [SMILES: COc1cc(NC(=O)OC(C)(C)C)nnc1C1CC(OCc2ccccc2)C1 ; COc1cc(N)nnc1C1CC(OCc2ccccc2)C1 ; "TFA"]

To a solution of tert-butyl N-[6-(3-benzyloxycyclobutyl)-5-methoxy-pyridazin-3-yl]carbamate (5.00 g, 13.0 mmol) in dichloromethane (50 mL) was added trifluoroacetic acid (1.48 g, 12.8 mmol, 0964 mL). The resulting reaction was stirred at room temperature for 16 hours, and was then concentrated under reduced pressure to provide the title compound: LCMS m/z 286.0 [M+H]⁺. ¹H NMR (400 MHz, CD₃OD) δ 7.37-7.21 (m, 6H), 4.45 (s, 2H), 4.21-4.07 (m, 1H), 4.00 (s, 3H), 3.27-3.18 (m, 1H), 2.70-2.56 (m, 2H), 2.36-2.10 (m, 2H).

### Step H. 6-(3-(benzyloxy)cyclobutyl)-3-(2-bromo-5-fluoropyrimidin-4-yl)-7-methoxyimidazo[1,2-b]pyridazine

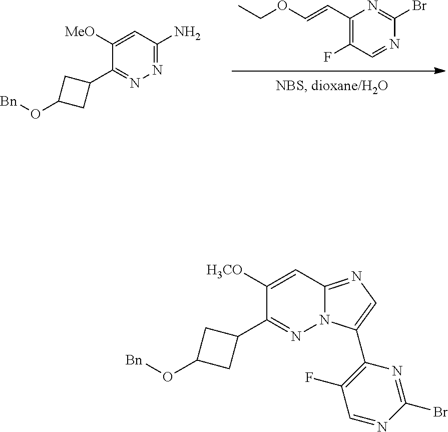

To a cooled 0° C. solution of 2-bromo-4-[(E)-2-ethoxyvinyl]-5-fluoro-pyrimidine (2.60 g, 10.5 mmol) in dioxane (3 mL) and water (1 mL) was added 1-bromopyrrolidine-2,5-dione (1.87 g, 10.5 mmol). The resulting reaction was stirred for 30 minutes while warming to room temperature. 6-(3-Benzyloxycyclobutyl)-5-methoxy-pyridazin-3-amine (2.00 g, 7.01 mmol) was added, and the reaction was then heated at 80° C. for 15 hours. The reaction was then cooled to room temperature, diluted with water (50 mL), and extracted with ethyl acetate (3×20 mL). The organic extracts were combined, washed with saturated aqueous sodium chloride solution (3×10 mL), dried over anhydrous sodium sulfate, filtered, and concentrated under reduced pressure. The resulting crude product was purified by flash chromatography on silica gel (0-100% ethyl acetate in petroleum ether) to provide the title compound LCMS m/z 484.05 [M+H]⁺; ¹H NMR (400 MHz, CD₃OD) δ 8.62 (d, J=3.0 Hz, 1H), 8.25 (s, 1H), 7.39-7.24 (m, 6H), 4.50 (s, 2H), 4.21-4.20 (m, 1H), 4.01 (s, 3H), 3.56-3.41 (m, 1H), 2.77-2.69 (m, 2H), 2.54-2.45 (m, 2H).

### Step I. tert-butyl (3S,4S)-3-((4-(6-(3-(benzyloxy)cyclobutyl)-7-methoxyimidazo[1,2-b]pyridazin-3-yl)-5-fluoropyrimidin-2-yl)amino)-4-fluoropiperidine-1-carboxylate

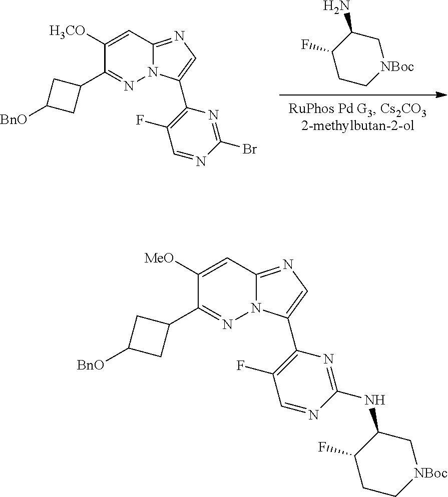

A mixture of 6-(3-benzyloxycyclobutyl)-3-(2-bromo-5-fluoro-pyrimidin-4-yl)-7-methoxy-imidazo[1,2-b]pyridazine (0.500 g, 1.03 mmol), tert-butyl (3S,4S)-3-amino-4-fluoro-piperidine-1-carboxylate (0.338 g, 1.55 mmol), (2-dicyclohexylphosphino-2′,6′-diisopropoxy-1,1′-biphenyl)[2-(2′-amino-1,1′ biphenyl)]palladium(II)methanesulfonate (0.086 g, 0.103 mmol), and cesium carbonate (0.841 g, 2.58 mmol) in 2-methylbutan-2-ol (5 mL) was purged with nitrogen, and was then heated at 80° C. for 5 hours under nitrogen atmosphere. The reaction was then cooled to room temperature, filtered, and concentrated under reduced pressure. The resulting crude product was purified by HPLC (Agela DuraShell C18 column, 10 micron, 250×70 mm; 55-80% acetonitrile in water containing 0.1% ammonium carbonate) to provide the title compound: LCMS m/z 622.3 [M+H]⁺.

### Step G. tert-butyl (3S,4S)-4-fluoro-3-((5-fluoro-4-(6-(3-hydroxycyclobutyl)-7-methoxyimidazo[1,2-b]pyridazin-3-yl)pyrimidin-2-yl)amino)piperidine-1-carboxylate

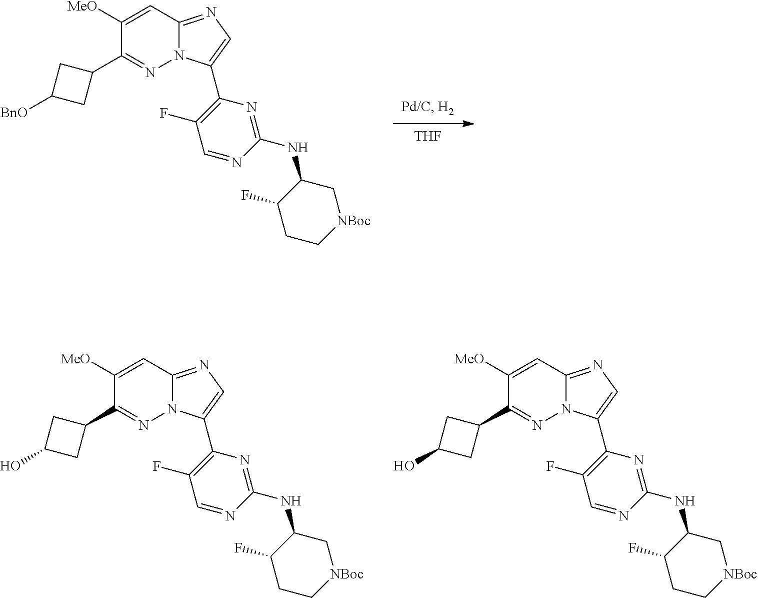 [SMILES: COc1cc2ncc(-c3nc(N[C@H]4CN(C(=O)OC(C)(C)C)CC[C@@H]4F)ncc3F)n2nc1C1CC(OCc2ccccc2)C1 ; COc1cc2ncc(-c3nc(N[C@H]4CN(C(=O)OC(C)(C)C)CC[C@@H]4F)ncc3F)n2nc1[C@H]1C[C@H](O)C1 ; COc1cc2ncc(-c3nc(N[C@H]4CN(C(=O)OC(C)(C)C)CC[C@@H]4F)ncc3F)n2nc1[C@H]1C[C@@H](O)C1 ; "THF"]

A mixture of tert-butyl (3S,4S)-3-[[4-[6-(3-benzyloxycyclobutyl)-7-methoxy-imidazo[1,2-b]pyridazin-3-yl]-5-fluoro-pyrimidin-2-yl]amino]-4-fluoro-piperidine-1-carboxylate (0.280 g, 0.450 mmol) and 10% palladium on carbon (2.88 g, 2.70 mmol, 10% purity) in tetrahydrofuran (2 mL) was purged with hydrogen, and was then stirred at room temperature for 2 hours under hydrogen atmosphere. The reaction mixture was then filtered and concentrated under reduced pressure. The resulting crude product was purified by HPLC (Waters Xbridge BEH C18 column, 10 micron, 100×30 mm; 20-50% acetonitrile in water containing 0.1% NH₄CO₃) to provide the title compounds: LCMS m/z 532.1 [M+H]⁺.

### Step K. (Trans)-3-(3-(5-fluoro-2-(((3S,4S)-4-fluoropiperidin-3-yl)amino)pyrimidin-4-yl)-7-methoxyimidazo[1,2-b]pyridazin-6-yl)cyclobutan-1-ol

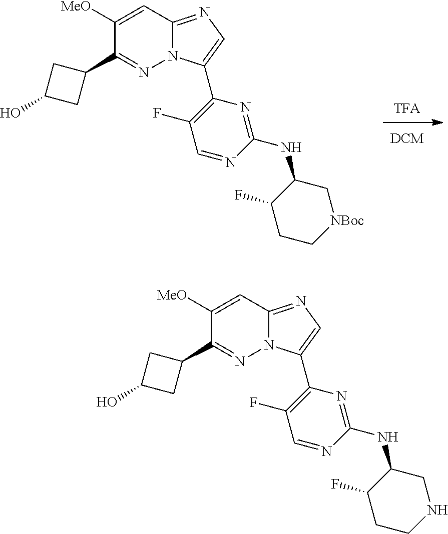 [SMILES: COc1cc2ncc(-c3nc(N[C@H]4CN(C(=O)OC(C)(C)C)CC[C@@H]4F)ncc3F)n2nc1[C@H]1C[C@H](O)C1 ; COc1cc2ncc(-c3nc(N[C@H]4CNCC[C@@H]4F)ncc3F)n2nc1[C@H]1C[C@H](O)C1]

To a solution of trans-3-(3-(5-fluoro-2-(((3S,4S)-4-fluoropiperidin-3-yl)amino)pyrimidin-4-yl)-7-methoxyimidazo[1,2-b]pyridazin-6-yl)cyclobutan-1-ol (0.015 g, 0.028 mmol) in dichloromethane (2 mL) was added trifluoroacetic acid (0.50 mL). The resulting reaction was stirred at room temperature for 30 minutes, and was then concentrated under reduced pressure. The crude product thus obtained was purified by HPLC (Waters Xbridge BEH C18 column, 3 micron, 100×30 mm; 25-55% acetonitrile in water containing 0.1% NH₄CO₃) to provide the title compound: LCMS m/z 432.0 [M+H]⁺; ¹H NMR (400 MHz, CD₃OD) δ 8.32 (d, J=2.9 Hz, 1H), 8.01 (d, J=1.8 Hz, 1H), 7.33 (s, 1H), 4.68-4.51 (m, 1H), 4.46 (t, J=6.6 Hz, 1H), 4.21 (br d, J=4.6 Hz, 1H), 4.00 (s, 3H), 3.90-3.77 (m, 1H), 3.28 (m, 1H), 3.10-3.00 (m, 1H), 2.81-2.61 (m, 3H), 2.58-2.50 (m, 1H), 2.44-2.30 (m, 2H), 2.21-2.07 (m, 1H), 1.82-1.67 (m, 1H).

### Step L. Cis-3-(3-(5-fluoro-2-(((3S,4S)-4-fluoropiperidin-3-yl)amino)pyrimidin-4-yl)-7-methoxyimidazo[1,2-b]pyridazin-6-yl)cyclobutan-1-ol

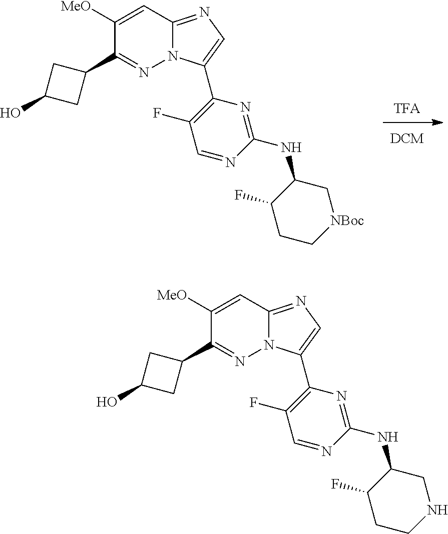 [SMILES: COc1cc2ncc(-c3nc(N[C@H]4CN(C(=O)OC(C)(C)C)CC[C@@H]4F)ncc3F)n2nc1[C@H]1C[C@@H](O)C1 ; COc1cc2ncc(-c3nc(N[C@H]4CNCC[C@@H]4F)ncc3F)n2nc1[C@H]1C[C@@H](O)C1]

To a solution of (cis)tert-butyl (3S,4S)-4-fluoro-3-[[5-fluoro-4-[6-(3-hydroxycyclobutyl)-7-methoxy-imidazo[1,2-b]pyridazin-3-yl]pyrimidin-2-yl]amino]piperidine-1-carboxylate (0.025 g, 0.047 mmol) in dichloromethane (2 mL) was added trifluoroacetic acid (0.50 mL). The resulting reaction was stirred at room temperature for 30 minutes, and was then concentrated under reduced pressure. The crude product thus obtained was purified by HPLC (Waters Xbridge BEH C18 column, 10 micron, 100×30 mm; 22-55% acetonitrile in water containing 0.1% NH₄CO₃) to provide the title compound: LCMS m/z 432.0 [M+H]⁺; ¹H NMR (400 MHz, CD₃OD) δ 8.32 (d, J=2.8 Hz, 1H), 8.00 (d, J=2.0 Hz, 1H), 7.31 (s, 1H), 4.73-4.54 (m, 1H), 4.69 (m, 1H), 4.34-4.24 (m, 1H), 4.23-4.14 (m, 1H), 3.99 (s, 3H), 3.45-3.34 (m, 2H), 3.12-2.98 (m, 1H), 2.78-2.65 (m, 3H), 2.63-2.55 (m, 1H), 2.39-2.25 (m, 2H), 2.16 (br d, J=3.8 Hz, 1H), 1.86-1.70 (m, 1H).

The compounds in Table 29 were all prepared using the synthetic procedures described in Example 30.

TABLE 29
Additional compounds prepared according to Example 30.

| Compound | | | |
|---|---|---|---|
| # | Structure | IUPAC Name | LCMS |
| 127a | 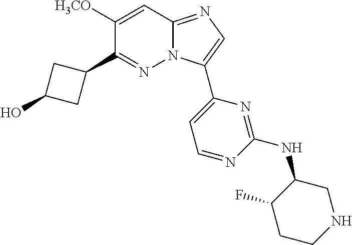 [SMILES: COc1cc2ncc(-c3ccnc(N[C@H]4CNCC[C@@H]4F)n3)n2nc1[C@H]1C[C@@H](O)C1] | (Cis)-3-(3-(2-(((3S,4S)-4- fluoropiperidin-3- yl)amino)pyrimidin-4-yl)-7- methoxyimidazo[1,2- b]pyridazin-6-yl)cyclobutan- 1-ol | 414.1 |

### Example 31

### Exemplary Synthetic Procedure #31 (Compounds 128a-129a)

### Compound 128a, 4-(6-cyclopropyl-7-(methoxy-d3)imidazo[1,2-b]pyridazin-3-yl)-N-((3S,4S)-4-fluoropiperidin-3-yl)pyrimidin-2-amine

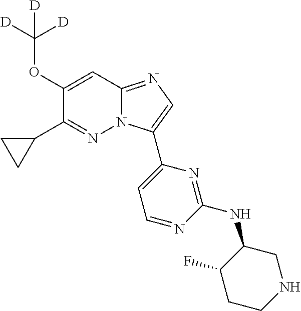 [SMILES: [2H]C([2H])([2H])Oc1cc2ncc(-c3ccnc(N[C@H]4CNCC[C@@H]4F)n3)n2nc1C1CC1]

### Step A. 6-cyclopropyl-7-methoxyimidazo[1,2-b]pyridazine

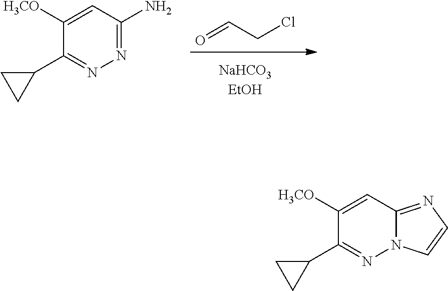

To a solution of 6-cyclopropyl-5-methoxy-pyridazin-3-amine (1.00 g, 6.05 mmol) and 2-chloroacetaldehyde (4.75 g, 60.5 mmol, 3.89 mL) in ethanol (2 mL) was added sodium bicarbonate (1.02 g, 12.1 mmol). The resulting reaction mixture was stirred at 80° C. for 4 hours, and was then cooled to room temperature and concentrated under reduced pressure. The resulting residue was diluted with water (10 mL) and extracted with ethyl acetate (3×10 mL). The combined organic layers were then washed with saturated aqueous sodium chloride solution (5 mL), dried over sodium sulfate, filtered, and concentrated under reduced pressure. The resulting crude product was purified by flash chromatography on silica gel (0-50% ethyl acetate in petroleum ether) to give the title compound: LCMS m/z 166.1 [M+H]⁺; ¹H NMR (400 MHz, CDCl₃) δ=7.64-7.52 (m, 1H), 7.40-7.29 (m, 1H), 7.02 (s, 1H), 3.88 (s, 3H), 2.44-2.19 (m, 1H), 1.10-1.02 (m, 2H), 1.00-0.92 (m, 2H).

### Step B. 6-cyclopropylimidazo[1,2-b]pyridazin-7-ol

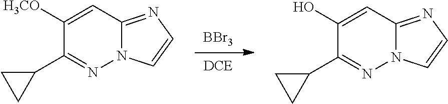

A solution of 6-cyclopropyl-7-methoxy-imidazo[1,2-b]pyridazine (5.00 g, 26.4 mmol) and tribromoborane (33.10 g, 132.1 mmol, 12.73 mL) in 1,2-dichloroethane (100 mL) was stirred at 60° C. for 2 hours under nitrogen atmosphere. The reaction was then cooled to room temperature, concentrated under reduced pressure, diluted with water (30 ml), and filtered. The collected solids were dried under reduced pressure to give the title compound: LCMS m/z 176.1 [M+H]⁺; ¹H NMR (400 MHz, CD₃OD) δ 8.05 (d, J=2.2 Hz, 1H), 7.82 (d, J=2.4 Hz, 1H), 7.31 (d, J=0.6 Hz, 1H), 2.58-2.53 (m, 1H), 1.23-1.18 (m, 4H).

### Step B. 6-cyclopropyl-7-(methoxy-d3)imidazo[1,2-b]pyridazine

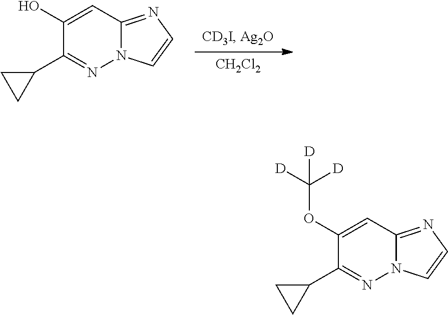

A mixture of 6-cyclopropylimidazo[1,2-b]pyridazin-7-ol (0.500 g, 2.85 mmol), iodomethane-ds (0.455 g, 3.14 mmol, 0.195 mL), and silver oxide (1.32 g, 5.71 mmol) in dichloromethane (5 mL) was purged with nitrogen, and was then heated at reflux for 16 hours under nitrogen atmosphere. The reaction mixture was then cooled to room temperature, filtered, and concentrated under reduced pressure to provide the title compound: LCMS m/z 193.1 [M+H]⁺; ¹H NMR (400 MHz, CDCl₃) δ 7.65 (s, 1H), 7.49 (s, 1H), 7.09 (s, 1H), 2.43-2.36 (m, 1H), 1.15-1.02 (m, 4H).

### Step C. 3-bromo-6-cyclopropyl-7-(methoxy-d3)imidazo[1,2-b]pyridazine

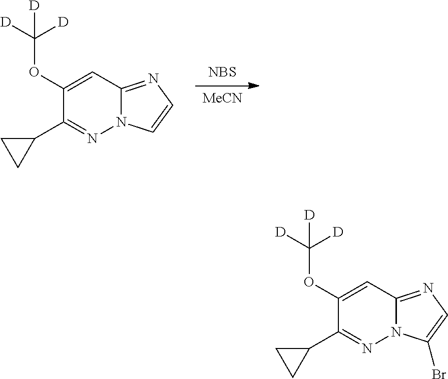

To a cooled 0° C. solution of 6-cyclopropyl-7-(trideuteriomethoxy)imidazo[1,2-b]pyridazine (0.680 g, 3.54 mmol) in acetonitrile (1 mL) was added 1-bromopyrrolidine-2,5-dione (0.693 g, 3.89 mmol). The resulting reaction was stirred for 2 hours while warming to room temperature. The reaction was then neutralized to pH ~7 by addition of aqueous sodium hydroxide solution (2 M, 5 mL) and extracted with ethyl acetate (10 mL). The organic extracts were combined, washed with saturated aqueous sodium chloride solution (3×10 mL), dried over anhydrous sodium sulfate, filtered, and concentrated under reduced pressure to provide the title compound: LCMS m/z 271.1 [M+H]⁺; ¹H NMR (400 MHz, CD₃OD) δ 7.45 (s, 1H), 7.19 (s, 1H), 2.52-2.38 (m, 1H), 1.19-1.06 (m, 4H).

### Step D. 3-(2-chloropyrimidin-4-yl)-6-cyclopropyl-7-(methoxy-d3)imidazo[1,2-b]pyridazine

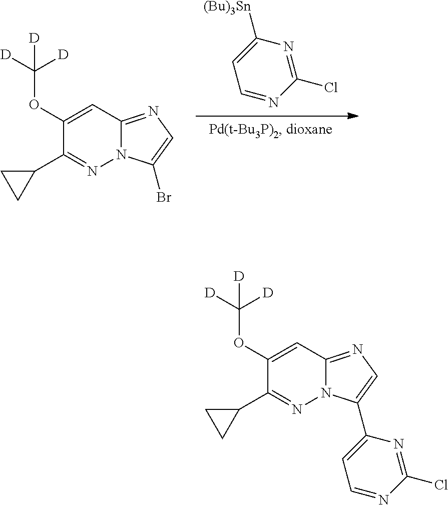

A mixture of 3-bromo-6-cyclopropyl-7-(trideuteriomethoxy)imidazo[1,2-b]pyridazine (0.520 g, 1.92 mmol), tributyl-(2-chloropyrimidin-4-yl)stannane (2.32 g, 5.75 mmol), and bis(tri-tert-butylphosphine)palladium (0.098 g, 0.192 mmol) in dioxane (10 mL) was purged with nitrogen, and was then heated at 100° C. for 16 hours under nitrogen atmosphere. The reaction was then cooled to room temperature, filtered, and concentrated under reduced pressure. The resulting crude product was purified by flash chromatography on silica gel (0-100% ethyl acetate in petroleum ether) to provide the title compound: LCMS m/z 305.1 [M+H]⁺.

### Step E. tert-butyl (3S,4S)-3-((4-(6-cyclopropyl-7-(methoxy-d3)imidazo[1,2-b]pyridazin-3-yl)pyrimidin-2-yl)amino)-4-fluoropiperidine-1-carboxylate (ET39539-1659)

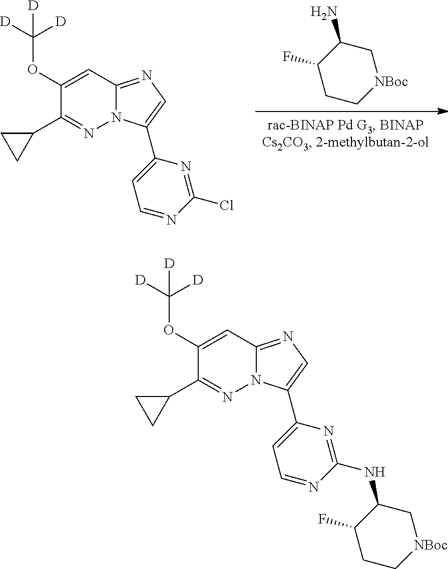

A mixture of 3-(2-chloropyrimidin-4-yl)-6-cyclopropyl-7-(trideuteriomethoxy)imidazo[1,2-b]pyridazine (0.040 g, 0.131 mmol), tert-butyl (3S,4S)-3-amino-4-fluoro-piperidine-1-carboxylate (0.043 g, 0.197 mmol), cesium carbonate (0.128 g, 0.394 mmol), and [2-(2-aminophenyl)phenyl]-methylsulfonyloxy-palladium [1-(2-diphenylphosphanyl-1-naphthyl)-2-naphthyl]-diphenyl-phosphane (0.013 g, 0.013 mmol) in 2-methylbutan-2-ol (2 mL) was purged with nitrogen, and was then heated at 80° C. for 3 hours under nitrogen atmosphere. The reaction was then cooled to room temperature, filtered, and concentrated under reduced pressure to provide the title compound: LCMS m/z 487.2 [M+H]⁺.

### Step F. 4-(6-cyclopropyl-7-(methoxy-d3)imidazo[1,2-b]pyridazin-3-yl)-N-((3S,4S)-4-fluoropiperidin-3-yl)pyrimidin-2-amine

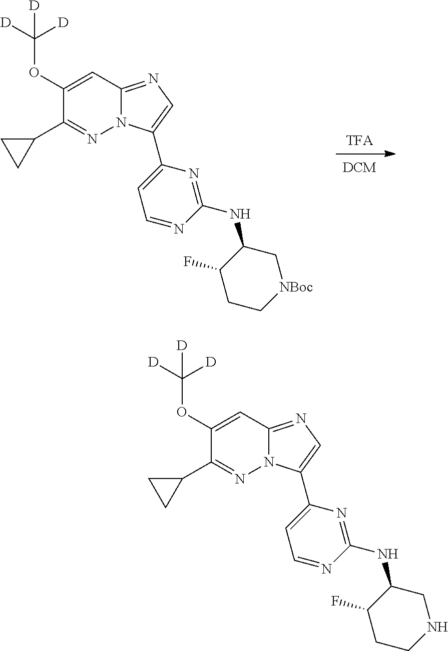

To a solution of tert-butyl (3S,4S)-3-[[4-[6-cyclopropyl-7-(trideuteriomethoxy)imidazo[1,2-b]pyridazin-3-yl]pyrimidin-2-yl]amino]-4-fluoro-piperidine-1-carboxylate (0.050 g, 0.103 mmol) in dichloromethane (1 mL) was added trifluoroacetic acid (0.50 mL). The resulting reaction was stirred at room temperature for 30 minutes, and was then concentrated under reduced pressure. The crude product thus obtained was purified by HPLC (Phenomenex Luna C18 column, 3 micron, 75×30 mm; 1-30% acetonitrile in water containing 0.1% trifluoroacetic acid) to provide the title compound: LCMS m/z 387.1 [M+H]⁺; ¹H NMR (400 MHz, CD₃OD) δ 8.60 (s, 1H), 8.54 (d, J=5.4 Hz, 1H), 7.83 (d, J=5.3 Hz, 1H), 7.61 (s, 1H), 4.96-4.83 (m, 1H), 4.63-4.50 (m, 1H), 3.68 (br d, J=12.8 Hz, 1H), 3.57-3.46 (m, 1H), 3.25-3.09 (m, 2H), 2.71-2.60 (m, 1H), 2.47 (br d, J=4.9 Hz, 1H), 2.11 (br s, 1H), 1.34-1.26 (m, 4H).

The compounds in Table 30 were all prepared using the synthetic procedures described in Example 31.

TABLE 30
Additional compounds prepared according to Example 31.

| Compound | | | |
|---|---|---|---|
| # | Structure | IUPAC Name | LCMS |
| 129a | 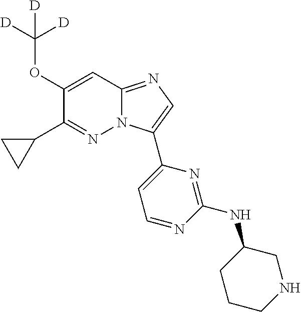 [SMILES: [2H]C([2H])([2H])Oc1cc2ncc(-c3ccnc(N[C@@H]4CCCNC4)n3)n2nc1C1CC1] | (R)-4-(6-cyclopropyl-7- (methoxy-d3)imidazo[1,2- b]pyridazin-3-yl)-N- (piperidin-3-yl)pyrimidin- 2-amine | 369.2 |

### Example 32

### Exemplary Synthetic Procedure #33 (Compounds 131a-134a)

### Compounds 131a & 132a, Fast-Eluting Diastereomer of 1,1,1-trifluoro-2-(3-(5-fluoro-2-(((R)-piperidin-3-yl)amino)pyrimidin-4-yl)imidazo[1,2-a]pyrazin-6-yl)propan-2-ol & Slow-Eluting Diastereomer of 1,1,1-trifluoro-2-(3-(5-fluoro-2-(((R)-piperidin-3-yl)amino)pyrimidin-4-yl)imidazo[1,2-a]pyrazin-6-yl)propan-2-ol

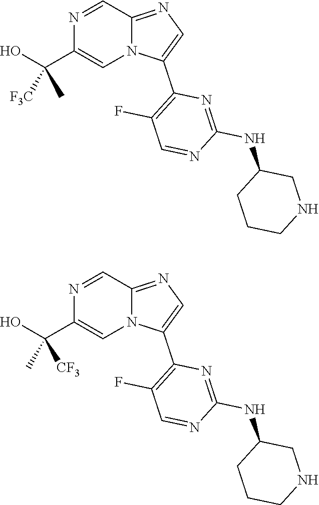 [SMILES: C[C@@](O)(c1cn2c(-c3nc(N[C@@H]4CCCNC4)ncc3F)cnc2cn1)C(F)(F)F ; C[C@](O)(c1cn2c(-c3nc(N[C@@H]4CCCNC4)ncc3F)cnc2cn1)C(F)(F)F]

### Step A. 1-(5aminopyrazin-2-yl)ethan-1-one

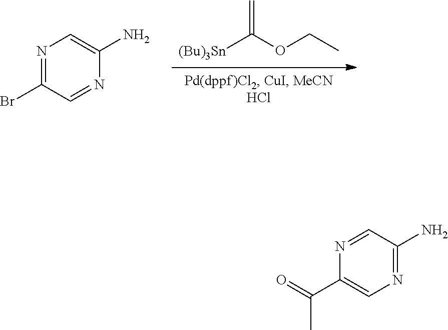

A mixture of 5-bromopyrazin-2-amine (5.00 g, 28.7 mmol), tributyl(1-ethoxyvinyl)stannane (31.13 g, 86.21 mmol, 29.12 mL), [1,1-bis(diphenylphosphino)ferrocene]dichloropalladium(II) (2.10 g, 2.87 mmol), and copper(I)iodide (0.821 g, 4.31 mmol) in acetonitrile (50 mL) was purged with nitrogen, and was then heated at 80° C. for 16 hours under nitrogen atmosphere. The reaction was then cooled to room temperature. Hydrochloric acid (2 M, 14.37 mL) was added, and the reaction was then stirred for 30 minutes, quenched by addition of aqueous potassium fluoride solution (20% w/w, 30 mL), and extracted with ethyl acetate (3×20 mL). The organic extracts were combined, washed with saturated aqueous sodium chloride solution (10 mL), dried over sodium sulfate, filtered, and concentrated under reduced pressure. The resulting crude product was purified by flash chromatography on silica gel (0-50% ethyl acetate in petroleum ether) to provide the title compound: LCMS m/z 138.4 [M+H]⁺; ¹H NMR (400 MHz, CD₃OD) δ 8.45 (d, J=1.3 Hz, 1H), 7.82 (d, J=1.1 Hz, 1H), 2.49-2.38 (s, 3H).

### Step B. 1-(3-(2-bromo-5-fluoropyrimidin-4-yl)imidazo[1,2-a]pyrazin-6-yl)ethan-1-one

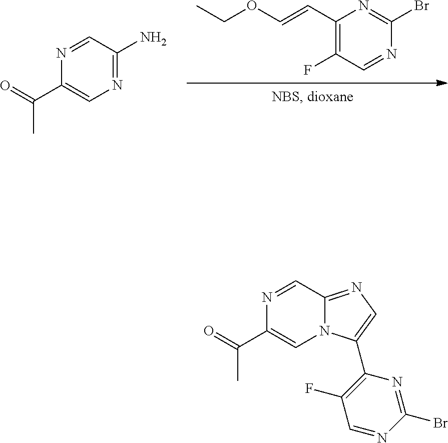

To a cooled 0° C. solution of 2-bromo-4-[(E)-2-ethoxyvinyl]-5-fluoro-pyrimidine (4.32 g, 17.5 mmol) in dioxane (30 mL) and water (10 mL) was added 1-bromopyrrolidine-2,5-dione (3.11 g, 17.5 mmol). The resulting reaction was stirred for 30 minutes while warming to room temperature. 1-(5-Aminopyrazin-2-yl)ethanone (1.60 g, 11.7 mmol) was added, and the reaction was then heated at 80° C. for 16 hours. The reaction was then diluted with aqueous sodium hydroxide solution (2 M, 30 mL) and extracted with ethyl acetate (3×20 mL). The organic extracts were combined, washed with saturated aqueous sodium chloride solution (10 mL), dried over sodium sulfate, filtered, and concentrated under reduced pressure. The resulting crude product was purified by flash chromatography on silica gel (0-50% ethyl acetate in petroleum ether) to provide the title compound: LCMS m/z 336.2 [M+H]⁺; ¹H NMR (400 MHz, CDCl₃) δ 10.36 (d, J=1.1 Hz, 1H), 9.24 (d, J=1.1 Hz, 1H), 8.64 (d, J=3.6 Hz, 1H), 8.50 (d, J=2.8 Hz, 1H), 2.77-2.73 (s, 3H).

### Step C. 2-(3-(2-bromo-5-fluoropyrimidin-4-yl)imidazo[1,2-a]pyrazin-6-yl)-1,1,1-trifluoropropan-2-ol

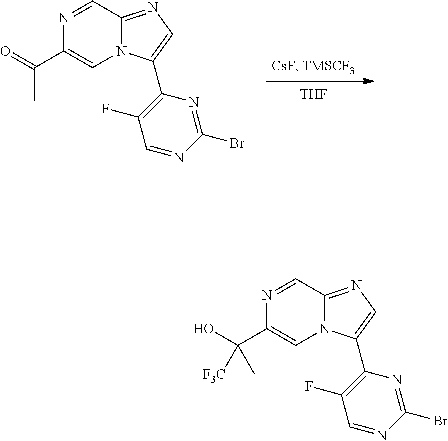

To a cooled 0° C. solution of 1-[3-(2-bromo-5-fluoro-pyrimidin-4-yl)imidazo[1,2-a]pyrazin-6-yl]ethanone (0.900 g, 2.68 mmol) and cesium fluoride (1.22 g, 8.03 mmol) in tetrahydrofuran (15 mL) was added (trifluoromethyl)trimethylsilane (3.43 g, 24.1 mmol). The resulting reaction was stirred for 2 hours while warming to room temperature. Hydrochloric acid (2 M, 2 mL) was added, and the reaction was stirred for 30 minutes, then diluted with water (40 mL) and extracted with ethyl acetate (3×40 mL). The organic extracts were combined, washed with saturated aqueous sodium chloride solution (40 mL), dried over sodium sulfate, filtered, and concentrated under reduced pressure. The resulting crude product was purified by flash chromatography on silica gel (0-50% ethyl acetate in petroleum ether) to provide the title compound: LCMS m/z 408.2 [M+H]⁺; ¹H NMR (400 MHz, CD30D) 8 10.15 (d, J=1.1 Hz, 1H), 9.13 (d, J=1.9 Hz, 1H), 8.73-8.49 (m, 2H), 1.80-1.76 (s, 3H).

### Step D. tert-butyl (3R)-3-((5-fluoro-4-(6-(1,1,1-trifluoro-2-hydroxypropan-2-yl)imidazo[1,2-a]pyrazin-3-yl)pyrimidin-2-yl)amino)piperidine-1-carboxylate

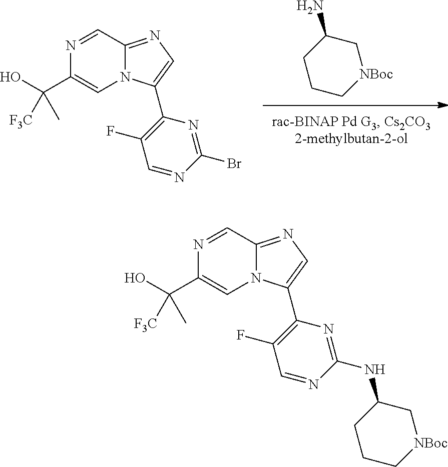 [SMILES: CC(O)(c1cn2c(-c3nc(Br)ncc3F)cnc2cn1)C(F)(F)F ; CC(C)(C)OC(=O)N1CCC[C@@H](N)C1 ; CC(C)(C)OC(=O)N1CCC[C@@H](Nc2ncc(F)c(-c3cnc4cnc(C(C)(O)C(F)(F)F)cn34)n2)C1]

A mixture of 2-[3-(2-bromo-5-fluoro-pyrimidin-4-yl)imidazo[1,2-a]pyrazin-6-yl]-1,1,1-trifluoro-propan-2-ol (0.050 g, 0.123 mmol), tert-butyl rac-(3R)-3-aminopiperidine-1-carboxylate (0.037 g, 0.185 mmol), methanesulfonato[2,2-bis(diphenylphosphino)-1,1-binaphthyl](2-amino-1,1-biphenyl-2-yl)palladium(II) (0.012 g, 0.012 mmol), [1-(2-diphenylphosphanyl-1-naphthyl)-2-naphthyl]-diphenyl-phosphane (0.008 g, 0.012 mmol), and cesium carbonate (0.120 g, 0.369 mmol) in 2-methylbutan-2-ol (5 mL) was purged with nitrogen, and was then heated at 80° C. for 16 hours under nitrogen atmosphere. The reaction was then cooled to room temperature, filtered, and concentrated under reduced pressure to the title compound: LCMS m/z 526.5 [M+H]⁺.

### Step E. Fast-Eluting Diastereomer of 1,1,1-trifluoro-2-(3-(5-fluoro-2-(((R)-piperidin-3-yl)amino)pyrimidin-4-yl)imidazo[1,2-a]pyrazin-6-yl)propan-2-ol & Slow-Eluting Diastereomer of 1,1,1-trifluoro-2-(3-(5-fluoro-2-(((R)-piperidin-3-yl)amino)pyrimidin-4-yl)imidazo[1,2-a]pyrazin-6-yl)propan-2-ol

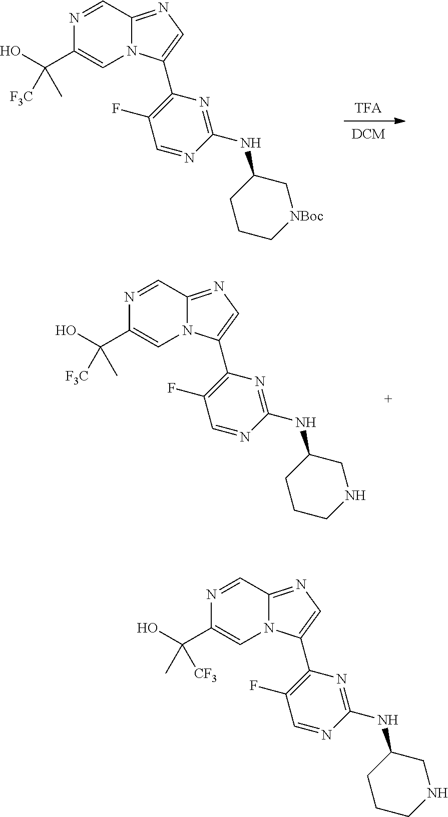

To a solution of tert-butyl rac-(3R)-3-[[5-fluoro-4-[6-(2,2,2-trifluoro-1-hydroxy-3-methyl-ethyl)imidazo[1,2-a]pyrazin-3-yl]pyrimidin-2-yl]amino]piperidine-1-carboxylate (0.160 g, 0.304 mmol) in dichloromethane (3 mL) was added trifluoroacetic acid (0.50 mL). The resulting reaction was stirred at room temperature for 2 hours, and was then concentrated under reduced pressure. The crude product thus obtained was purified by HPLC (Phenomenex Luna C18 column, 3 micron, 75×30 mm; 10-30% acetonitrile in water containing 0.1% trifluoroacetic acid) to provide the title compounds as two diastereomers. Fast-eluting diastereomer: LCMS m/z 426.2 [M+H]⁺; ¹H NMR (400 MHz, CD₃OD) δ 10.23 (s, 1H), 9.09 (d, J=1.3 Hz, 1H), 8.49 (d, J=3.5 Hz, 1H), 8.36 (d, J=3.5 Hz, 1H), 4.22 (br s, 1H), 3.50 (dd, J=3.3, 12.5 Hz, 1H), 3.25-3.22 (m, 1H), 3.17-3.01 (m, 2H), 2.15-2.02 (m, 2H), 1.83 (s, 5H). Slow-eluting diastereomer: LCMS m/z 426.2 [M+H]⁺; ¹H NMR (400 MHz, CD₃OD) δ 10.24 (s, 1H), 9.19 (d, J=0.9 Hz, 1H), 8.57 (d, J=3.5 Hz, 1H), 8.46 (d, J=3.4 Hz, 1H), 4.34 (br s, 1H), 3.62-3.51 (m, 1H), 3.32-3.10 (m, 3H), 2.18 (br s, 2H), 2.01-1.84 (m, 5H).

The compounds in Table 31 were all prepared using the synthetic procedures described in Example 32.

TABLE 31
Additional compounds prepared according to Example 32.

| Compound | | | |
|---|---|---|---|
| # | Structure | IUPAC Name | LCMS |
| 133a | 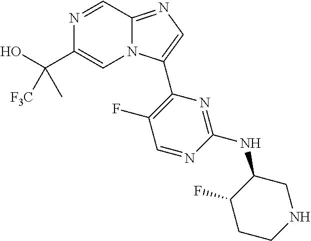 [SMILES: CC(O)(c1cn2c(-c3nc(N[C@H]4CNCC[C@@H]4F)ncc3F)cnc2cn1)C(F)(F)F] | Fast-eluting diastereomer- 1,1,1-trifluoro-2-(3-(5- fluoro-2-(((3S,4S)-4- fluoropiperidin-3- yl)amino)pyrimidin-4- yl)imidazo[1,2-a]pyrazin- 6-yl)propan-2-ol | 444.2 |
| 134a | 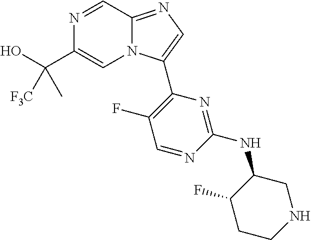 [SMILES: CC(O)(c1cn2c(-c3nc(N[C@H]4CNCC[C@@H]4F)ncc3F)cnc2cn1)C(F)(F)F] | Slow-eluting diastereomer-1,1,1- trifluoro-2-(3-(5-fluoro- 2-(((3S,4S)-4- fluoropiperidin-3- yl)amino)pyrimidin-4- yl)imidazo[1,2-a]pyrazin- 6-yl)propan-2-ol | 444.2 |

### Example 33

### Biological Data for Exemplary Compounds

Kinase inhibitory data were obtained for various exemplary compounds in Examples 3-32 using the RBC HotSpot Kinase Assay Protocol (Anastassiadis T, et al. Comprehensive assay of kinase catalytic activity reveals features of kinase inhibitor selectivity. Nat Biotechnol. 2011 Oct. 30; 29(11):1039-45), as described below. This assay uses the isolated kinase enzyme. This assay is very useful for determining competition of the inhibitor for ATP and/or substrates and for measuring the kinetics of enzyme inhibition. It also allows for measuring the relative affinity of binding to the isolated enzyme protein, and hence determines selectivity. Unlike kinase binding assays that measure competition for ATP, the HotSpot Kinase Assay is a functional assay that measures catalytic activity; as such it measures relative functional potency regardless of the mechanism of enzyme inhibition. This assay uses the form of the various enzymes that are easiest to express, which may not necessarily be the form of the enzyme that exist in the cell. (Sometimes the carboxy terminus has been truncated to aid in expression, or, if it is a receptor kinase, the enzyme itself is isolated from the other parts of the receptor that are involved in regulating kinase activity.)

The reagent used was as follows: Base Reaction buffer; 20 mM Hepes (pH 7.5), 10 mM MgCl2, 1 mM EGTA, 0.01% Brij35, 0.02 mg/ml BSA, 0.1 mM Na3VO4, 2 mM DTT, 1% DMSO. Required cofactors were added individually to each kinase reaction.

The reaction procedure was as follows:
1) Substrates were prepared in freshly prepared Reaction Buffer.
2) Any required cofactors were delivered to the substrate solution above.
3) Kinase was delivered into the substrate solution and gently mixed.
4) Compounds were delivered in 1000A DM into the kinase reaction mixture by Acoustic technology (Echo550; nanoliter range), followed by incubation for 20 min at room temp.
5) 33P-ATP was delivered into the reaction mixture to initiate the reaction.
6) The mixture was incubated for 2 hours at room temperature.
7) Kinase activity was detected by P81 filter-binding method.

Table 32. Biological data obtained in accordance with the protocol described in Example 33.

TABLE 32
Biological data obtained in accordance with the
protocol described in Example 33.

| Com- | IRAK1 IC₅₀ | IRAK4 IC₅₀ | FLT3 IC₅₀ |
|---|---|---|---|
| pound # | (nM) | (nM) | (nM) |
| 1a | 1 | 0.9 | 0.5 |
| 2a | 1 | 0.7 | <0.5 |
| 3a | 2 | <0.5 | <0.5 |
| 4a | 31 | 1 | <0.5 |
| 5a | 29 | 0.9 | <0.5 |
| 6a | 133 | 3 | <0.5 |
| 7a | 73 | 2 | <0.5 |
| 8a | 34 | 2 | <0.5 |
| 9a | 199 | 11 | <0.5 |
| 10a | 26 | 3 | <0.5 |
| 11a | 52 | 5 | <0.5 |
| 12a | 18 | 6 | <0.5 |
| 13a | 55 | 5 | <0.5 |
| 14a | 117 | 16 | <0.5 |
| 15a | 258 | 18 | <0.5 |
| 16a | 245 | 8 | <0.5 |
| 17a | 1420 | 215 | 4 |
| 18a | 226 | 15 | <0.5 |
| 19a | 70 | 13 | <0.5 |
| 20a | 170 | 12 | 0.7 |
| 21a | 236 | 81 | <0.5 |
| 22a | 19 | 3 | 0.6 |
| 23a | 3 | 0.8 | <0.5 |
| 24a | 16 | 0.9 | <0.5 |
| 25a | 17 | 2 | <0.5 |
| 26a | 5 | <0.5 | <0.5 |
| 27a | 4 | 0.7 | <0.5 |
| 28a | 33 | 3 | 2 |
| 29a | 27 | 0.8 | 1 |
| 30a | 108 | 5 | 2 |
| 31a | 17 | 2 | 1 |
| 32a | 53 | 1 | 2 |
| 33a | 9 | 0.8 | 0.7 |
| 34a | 4 | 2 | 0.6 |
| 35a | 0.9 | 0.7 | <0.5 |
| 36a | 4 | 2 | <0.5 |
| 37a | 2 | 2 | <0.5 |
| 38a | 1 | 0.6 | <0.5 |
| 39a | 2 | 1 | <0.5 |
| 40a | 1130 | 76 | 3 |
| 41a | 1590 | 36 | 1 |
| 42a | 3250 | 496 | 10 |
| 43a | 50 | 29 | 1 |
| 44a | 32 | 4 | <0.5 |
| 45a | 18 | 8 | <0.5 |
| 46a | 6 | 2 | <0.7 |
| 47a | 13 | 1 | <0.5 |
| 48a | 10 | 5 | 1 |
| 49a | 2 | 0.5 | <0.5 |
| 50a | 3 | 0.8 | <0.5 |
| 51a | 3 | 3 | <0.5 |
| 52a | 0.8 | 0.9 | <0.5 |
| 53a | 1 | <0.5 | <0.5 |
| 54a | <0.5 | <0.5 | <0.5 |
| 55a | 5 | 2 | <0.5 |
| 56a | 0.7 | 2 | <0.5 |
| 57a | 2 | 1 | <0.5 |
| 58a | 7 | 5 | <0.5 |
| 59a | 0.7 | 3 | <0.5 |
| 60a | 4 | 8 | <0.5 |
| 61a | <0.5 | <0.5 | <0.5 |
| 62a | 0.6 | <0.5 | <0.5 |
| 63a | <0.5 | <0.5 | <0.5 |
| 64a | <0.5 | <0.5 | <0.5 |
| 65a | <0.5 | <0.5 | <0.5 |
| 66a | <0.5 | 0.6 | <0.5 |
| 67a | 1 | 0.9 | <0.5 |
| 68a | 0.9 | 0.8 | <0.5 |
| 69a | 0.9 | 0.7 | <0.5 |
| 70a | 3 | <0.5 | <0.5 |
| 71a | 342 | 64 | 1 |
| 72a | 127 | 60 | 0.8 |
| 73a | 129 | 12 | <0.5 |
| 74a | 91 | 26 | <0.5 |
| 75a | 102 | 6 | <0.5 |
| 76a | 37 | 21 | <0.5 |
| 77a | 12 | 1 | <0.5 |
| 78a | 75 | 7 | <0.5 |
| 79a | 33 | 2 | <0.5 |
| 80a | 142 | 17 | <0.5 |
| 81a | 49 | 55 | <0.5 |
| 82a | 174 | 45 | <0.5 |
| 83a | 8 | 0.6 | <0.5 |
| 84a | 18 | 2 | <0.5 |
| 85a | 7 | 1 | <0.5 |
| 86a | 16 | 1 | <0.5 |
| 87a | 26 | 3 | <0.5 |
| 88a | 13 | 4 | <0.5 |
| 89a | 44 | 4 | <0.5 |
| 90a | 85 | 9 | <0.5 |
| 91a | 45 | 5 | <0.5 |
| 92a | 4 | <0.5 | <0.5 |
| 93a | 5 | <0.5 | <0.5 |
| 94a | 28 | 4 | <0.5 |
| 95a | 300 | 6 | 0.7 |
| 96a | 1790 | 62 | 0.5 |
| 97a | 361 | 4 | 0.7 |
| 98a | 46 | 2 | 1 |
| 99a | 44 | 11 | 3 |
| 100a | 24 | 2 | 1 |
| 101a | 24 | 1 | 0.8 |
| 102a | 81 | 2 | <0.5 |
| 103a | 179 | 3 | 0.7 |
| 104a | 4860 | 25 | 14 |
| 105a | >10000 | 259 | 33 |
| 106a | 18 | 6 | <0.5 |
| 107a | 102 | 20 | 0.6 |
| 108a | 241 | 57 | 2 |
| 109a | 239 | 4 | 1 |
| 110a | 23 | 20 | <0.5 |
| 111a | 40 | 3 | <0.5 |
| 112a | 19 | 3 | <0.5 |
| 117a | 397 | 45 | 3 |
| 118a | 732 | 155 | 7 |
| 119a | 1 | <0.5 | <0.5 |
| 120a | 10 | 10 | <0.5 |
| 121a | 1 | 0.7 | <0.5 |
| 122a | 22 | 18 | <0.5 |
| 123a | 43 | 5 | <0.5 |
| 124a | 123 | 138 | 2 |
| 125a | <0.5 | <0.5 | <0.5 |
| 126a | 37 | <0.5 | <0.5 |
| 127a | 20 | <0.5 | <0.5 |
| 128a | 24 | 0.7 | <0.5 |
| 129a | 86 | 2 | <0.5 |
| 131a | 15 | 28 | 2 |
| 132a | 3 | 2 | <0.5 |
| 133a | 15 | 15 | 1 |
| 134a | 2 | 1 | <0.5 |

### Example 34

### Biological Data for Exemplary Compounds

Kinase binding data were obtained for various exemplary compounds in Examples 3-32 using the DiscoverX KINOMEscan® active site-directed competition binding site-directed assay protocol described below. Unlike other kinase competitive binding site assays, KINOMEscan® assays do not require ATP. As a result, the data report thermodynamic interaction affinities (K_{d} values), rather than IC₅₀ values that are dependent on ATP concentrations. The assay uses a DNA-tagged version of the protein kinase, and an immobilized ligand bound to a solid support. Compounds that directly or indirectly prevent kinase binding to the immobilized ligand reduce the amount of kinase captured on the solid support, which is detected using an ultra-sensitive qPCR method. Affinity constants reported from the assay have been reported to be independent of the immobilized ligand used that is coupled to the solid support (See supplemental information in Fabian, M. A. et. al., (2005) Nat. Biotechnol. 23, 329-336; Wodicka, L. M. et. al., (2010) Chem. Biol. 17, 1241-1249.)

Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17x PBS, 0.05% Tween 20, 6 mM DTT). Test compounds were prepared as 111× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions were performed in polypropylene 384-well plates. Each was a final volume of 0.02 mL. The assay plates were incubated at room temperature with shaking for 1 hour and the affinity beads were washed with wash buffer (lx PBS, 0.05% Tween 20). The beads were then re-suspended in elution buffer (lx PBS, 0.05% Tween 20, 0.5 μM nonbiotinylated affinity ligand) and incubated at room temperature with shaking for 30 minutes. The kinase concentration in the eluates was measured by qPCR.

Binding constants (Kds) were calculated with a standard dose-response curve using the Hill equation. The Hill Slope was set to −1. Curves were fitted using a non-linear least square fit with the Levenberg-Marquardt algorithm.

TABLE 33
Biological data obtained in accordance with the protocol described in Example 34.

| | IRAK1 | IRAK4 | FLT3 | FLT3 | FLT3 | FLT3 | FLT3 |
|---|---|---|---|---|---|---|---|
| | K_{d} | K_{d} | K_{d} | D835H | D835V | D835Y | ITD K_{d} |
| Compound # | (nM) | (nM) | (nM) | K_{d} (nM) | K_{d} (nM) | K_{d} (nM) | (nM) |
| 1a | <0.17 | <0.17 | <0.17 | 0.22 | <0.17 | 0.23 | 0.21 |
| 4a | 7.8 | 0.59 | <0.17 | 0.7 | <0.17 | 0.42 | 1.7 |
| 45a | 1.6 | 0.58 | 1.1 | 0.42 | 0.87 | 0.2 | <0.17 |
| 46a | 0.7 | <0.17 | 1.2 | 0.67 | 0.53 | 0.26 | 0.19 |
| 92a | 0.49 | <0.17 | <0.17 | <0.17 | <0.17 | <0.17 | 0.21 |
| 100a | 4.5 | 1.5 | 16 | 6.3 | 2.1 | 2.4 | 2.7 |
| 106a | 1.1 | 0.98 | 0.63 | <0.17 | <0.17 | <0.17 | 0.21 |
| 112a | 1.1 | 0.61 | <0.17 | <0.17 | <0.17 | <0.17 | 0.2 |

TABLE 34
Biological data obtained in accordance with the protocol described in Example 34.

| | FLT3 | FLT3 | FLT3 | FLT3 | FLT3 | FLT3 |
|---|---|---|---|---|---|---|
| | ITD, D835V | ITD, F691L | K663Q | N841I | R834Q K_{d} | Autoinh. |
| Compound # | K_{d} (nM) | K_{d} (nM) | K_{d} (nM) | K_{d} (nM) | (nM) | K_{d} (nM) |
| 1a | <0.17 | <0.17 | 0.27 | <0.17 | <0.17 | 2.2 |
| 4a | <0.17 | <0.17 | 2.3 | 0.88 | 0.69 | 21 |
| 45a | 0.23 | 0.19 | 3.5 | 1.1 | 5.9 | 11 |
| 46a | <0.17 | 0.22 | 3.7 | 3.2 | 4.4 | 7.1 |
| 92a | <0.17 | <0.17 | 0.49 | 0.18 | 0.77 | 3.3 |
| 100a | 0.19 | 3.4 | 30 | 16 | 40 | 57 |
| 106a | <0.17 | <0.17 | 1.5 | 0.53 | 1.9 | 12 |
| 112a | <0.17 | <0.17 | 0.31 | 0.2 | 0.28 | 2.7 |

### Example 35

### Biological Data for Exemplary Compounds

Kinase cellular potency data were obtained for various exemplary compounds in Examples 3-32 using the Reaction Biology NanoBRET assay protocol described below. The NanoBRET assay measures kinase engagement in real time in the context of the intact cell. Unlike the previously described biochemical kinase assay methodology in Example 33, the NanoBRET assay measures the binding and activity characteristics under equilibrium conditions using full-length kinases in the presence of cellular concentrations of ATP in live, uncompromised cells. As such, the assay provides a more relevant assessment of kinase potency and selectivity that would be expected to be observed in the native cellular environment, where potency is often considerably lower than that observed in the isolated biochemical assays (Vasta, J. D. et al., (2018) Cell Chem. Biol. 25, 206-214). The assay uses a Kinase-NanoLuc® fusion vector expressing a kinase protein to which a luciferase tag has been added, a cell-permeant fluorescent NanoBRET™ tracer, a NanoLuc® substrate, and an extracellular NanoLuc® inhibitor. Upon expression of the luciferase-tagged kinase, cells will produce a strong BRET signal only in the presence of the NanoBRET™ tracer. The extracellular NanoLuc® inhibitor ensures that the BRET signal observed emanates only from live cells. Because the BRET signal has tight distance constraints, addition of the test compound will decrease the BRET signal if the compound competes with the NanoBRET™ tracer for binding to the kinase domain. Under the appropriate tracer conditions established by the manufacturer, quantitative intracellular affinity and relative potency can then be determined using Mass Action model equations.

HEK-293 cells were purchased from ATCC. FuGENEHD Transfection Reagent, Kinase-NanoLucfusion plasmids, Transfection Carrier DNA, NanoBRETTracers and dilution buffer, NanoBRETNano-Glo Substrate, Extracellular NanoLucInhibitor were obtained from Promega.

Assays were conducted following Promega assay protocol with some modifications. HEK-293 Cells were transiently transfected with Kinase-NanoLucFusion Vector DNA by FuGENEHD Transfection Reagent. Testing compounds were delivered into 384 well assay plate by Echo 550 (LabcyteInc, Sunnyvale, CA). Transfected cells were harvested and mixed with NanoBRETTracer Reagent and dispensed into 384 well plates and incubated at 37° C. in 5% C₀₂ cell culture incubator for 1 hour. The NanoBRETNano-Glo Substrate plus Extracellular NanoLucInhibitor Solution were added into the wells of the assay plate and incubated for 2-3 minutes at room temperature. The donor emission wavelength (460 nm) and acceptor emission wavelength (600 nm) were measured in the EnVisionplate reader. The BRET Ratios were calculated. BRET Ratio=[(Acceptor sample÷Donor sample)−(Acceptor no-tracer control÷Donor no-tracer control)]. The IC₅₀ values of compounds were calculated with Prism GraphPad program.

### NanoBRET™ Target Engagement Assay Protocol

### 1. Transient Transfection of HEK-293 Cells NanoLuc® Fusion Vector DNA

1). Cultivate HEK-293 cells (70-80% confluence) appropriately prior to assay. Trypsinize and collect HEK-293 cells.
2). Prepare lipid: DNA complexes as follows:
   a. Prepare a 10 μg/ml solution of DNA in Opti-MEM without serum that consists of the following ratios of carrier DNA and DNA encoding NanoLuc® fusion. 9.0 μg/mL of Transfection Carrier DNA, 1.0 μg/mL of NanoLuc fusion vector DNA and 1 mL of Opti-MEM without phenol red. Mix thoroughly.
   b. Add 30 μl of FuGENE HD Transfection Reagent into each milliliter of DNA mixture to form lipid: DNA complex.
   c. Mix by inversion 10 times.
   d. Incubate at ambient temperature for 20 minutes to allow complexes to form.
3). In a sterile, conical tube, mix 1 part of lipid:DNA complex with 20 parts of HEK-293 cells in suspension. Mix gently by inversion 5 times.
4). Dispense cells+lipid:DNA complex into a sterile tissue culture dish and incubate for 22-24 hours.

### 2. Addition of Test Compounds (Dry Plate Shooting)

Each test compound is delivered from the compound source plate to the wells of 384-well white NBS plate by Echo 550.

3. Preparation of Cells with NanoBRET™ Tracer Reagent
1). Remove medium from dish with transfected HEK-293 cells via aspiration, trypsinize and allow cells to dissociate from the dish.
2). Neutralize trypsin using medium containing serum and centrifuge at 200×g for 5 minutes to pellet the cells. Adjust the cell density to 2×105 cells/mL in Opti-MEM without phenol red.
3). Prepare Complete 20× NanoBRET™ Tracer Reagent with Tracer Dilution Buffer.
4). Dispense one part of Complete 20× NanoBRET™ Tracer Reagent to 20 parts of cells in the tube. Mix gently by inversion 10 times.
5). Dispense cell suspension into white, 384-well NBS plates. Incubate the plate at 37° C., 5% CO₂ for 1 hour.

Note: Prepare a separate set of samples without tracer for background correction steps.

### 4. NanoBRET™ Assay

1). Remove plate from incubator and equilibrate to room temperature for 15 minutes.
2). Prepare 3× Complete Substrate plus Inhibitor Solution in Assay Medium (Opti-MEMR I Reduced Serum Medium, no phenol red) just before measuring BRET.
3). Add 3X Complete Substrate plus Inhibitor Solution to each well of the 384-well plate. Incubate for 2-3 minutes at room temperature.
4). Measure donor emission wavelength (460 nm) and acceptor emission wavelength (600 nm) using the Envision 2104 plate reader.

### 5. Determination of BRET Ratio

To generate raw BRET ratio values, divide the acceptor emission value (600 nm) by the donor emission value (460 nm) for each sample. To correct for background, subtract the BRET ratio in the absence of tracer (average of no-tracer control samples) from the BRET ratio of each sample.

BRET Ratio=(Acceptor sample+Donor sample) NanoBRET™ Ratio Equation:

BRET Ratio=[(Acceptor sample÷Donor sample)−(Acceptor no-tracer control÷Donor no-tracer control)] NanoBRET™ Ratio Equation, Including Optional Background Correction:

(BRET Ratio of Compound Treated Sample/BRET Ratio of DMSO Control Sample)*100% Normalized Bret Response Equation (%):

### 6. Determination of IC₅₀ Values

IC₅₀ curves are plotted and IC₅₀ values are calculated using the GraphPad Prism 4 program based on a sigmoidal dose-response equation.

TABLE 35
Biological data obtained in accordance with the protocol
described in Example 35.

| | NanoBRET IRAK4 | NanoBRET FLT3 |
|---|---|---|
| Compound # | IC₅₀ (nM) | IC₅₀ (nM) |
| 1a | <0.5 | 1 |
| 4a | 0.8 | 4 |
| 12a | <0.5 | <0.5 |
| 22a | <0.5 | 38 |
| 23a | <0.5 | 3 |
| 25a | 2 | 40 |
| 28a | <0.5 | 2960 |
| 31a | <0.5 | 1810 |
| 45a | 3 | 44 |
| 46a | 4 | 102 |
| 48a | 5 | 141 |
| 49a | <0.5 | <0.5 |
| 79a | <0.5 | <0.5 |
| 92a | <0.5 | 4 |
| 100a | 8 | 4250 |
| 106a | <0.5 | 14 |
| 111a | 34 | 77 |
| 112a | <0.5 | <0.5 |

### Example 36

### Biological Data for Exemplary Compounds

Cellular potency data were obtained for various exemplary compounds in Examples 3-32 using the NF-kB assay protocol described below. Activation of NF-kB gene transcription is a downstream signal in the IRAK signaling pathway (Balka, K. R. and DeNardo, D., J. Leukoc. Biol. (2019) 105, 339-351. Because THP-1 cells do not contain activated FLT3 receptors, measurement of the ability of a FLT3/IRAK1/IRAK4 inhibitor compound to inhibit the NF-kB production reflects the ability to inhibit signaling downstream of blocking signaling through the IRAK1/4 complex, and is not a composite measurement of activity that includes FLT3 kinase inhibition.

THP-1-Blue NF-κB cells (InvivoGen) carrying a stable integrated NF-κB-inducible secreted embryonic alkaline phosphatase (SEAP) reporter construct were plated at a concentration of 1×10⁵ cells per well. The cells were stimulated with Pam3CSK4 (1 ng/mL) or hIL1B (1 ng/mL). After 10-20 minutes, the cells were then treated with vehicle (DMSO) or serial dilutions of the test compounds (10 doses tested for each test compound, with a 1:10 dilution series starting at 1 μM or 3 μM) with a final volume of 200 μL for 24 hours at 37° C. After 24 hours, the cells were centrifuged and 20 μL supernatant was incubated with 180 μL QUANTI-Blue reagent at 37° C. for 30-60 minutes. The levels of NF-κB-induced was measured in a microplate reader at 620 nm.

TABLE 36
Biological data obtained in accordance with the protocol
described in Example 36.

| Com- | NF-κB Pam3SCK4 | NF-κB IL1B |
|---|---|---|
| pound # | IC₅₀ (nM) | IC₅₀ (nM) |
| 1a | 17 | 22 |
| 3a | 40 | 58 |
| 4a | 184 | 166 |
| 12a | 382 | 220 |
| 13a | 512 | 334 |
| 19a | 701 | 567 |
| 23a | 56 | 96 |
| 25a | 193 | 152 |
| 27a | 55 | 52 |
| 28a | 96 | 149 |
| 31a | 138 | 100 |
| 37a | 70 | 58 |
| 45a | 204 | 351 |
| 46a | 103 | 112 |
| 48a | 56 | 96 |
| 49a | 79 | 89 |
| 51a | 136 | 135 |
| 57a | 157 | 167 |
| 59a | 250 | 323 |
| 60a | 156 | 200 |
| 68a | 144 | 108 |
| 79a | 340 | 271 |
| 92a | 53 | 60 |
| 94a | 188 | 206 |
| 100a | 69 | 83 |
| 106a | 305 | 462 |
| 112a | 235 | 355 |
| 119a | 57 | 69 |
| 120a | 339 | 217 |
| 121a | 89 | 57 |
| 122a | 407 | 407 |
| 125a | 30 | 29 |
| 126a | 192 | 183 |

### Example 37

### Biological Data for Exemplary Compounds

Cellular potency data were obtained for various exemplary compounds in Examples 3-32 using the MOLM14 D835Y and MOLM14 F691L cell viability assay protocols described below. Both cell lines have activated FLT3 receptors, each of which carry additional resistance mutations in the kinase domain (D835Y and F691L, respectively). Leukemias from patients harboring these kinase domain resistance mutations are resistant to FLT3 inhibitors that do not inhibit the mutant kinase. Because the activated FLT3 receptor drives a mitogenic response, and because there can be a discrepancy between activity in the biochemical kinase assay and in the context of a whole cell (Vasta, J. D. et al., (2018) Cell Chem. Biol. 25, 206-214), demonstration of antiproliferative activity in these cell lines with compounds known to inhibit the D835Y or F691L kinases in biochemical assays provides a more relevant cellular context for demonstration of activity.

MOLM14 D835Y and MOLM14 F691L cells were grown in RPMI-1640 media supplemented with 20% fetal bovine serum (FBS). For viability/cytotoxicity assessments, cells were seeded into 1536-well white polystyrene tissue culture-treated Greiner plates using a Multidrop Combi dispenser (ThermoFisher), in final volume 5 μL of growth media per well, at a density of 1000 cells per well. After cell addition, 23 nL of test compound were transferred into individual wells (22 doses tested for each test compound, with a 1:2 dilution series starting at 10 M) via a 1536 pin-tool. Bortezomib (final concentration 2.3 μM) was used as a positive control for cell cytotoxicity. Plates were incubated for 48 hours at standard incubator conditions covered by a stainless steel gasketed lid to prevent evaporation. 48 hours post compound addition, 3 μL of Cell Titer Glo (Promega) were added to each well and plates were incubated at room temperature for 15 minutes with the stainless-steel lid in place. Luminescence readings were taken using a Viewlux imager (PerkinElmer) with a 2 second exposure time per plate.

TABLE 37
Biological data obtained in accordance with the protocol
described in Example 37.

| Com- | MOLM14 | MOLM14 F691L |
|---|---|---|
| pound # | D835Y IC₅₀ (nM) | IC₅₀ (nM) |
| 1a | 1 | 6 |
| 2a | 4 | N.D. |
| 3a | 1 | 5 |
| 4a | 9 | 36 |
| 5a | 29 | N.D. |
| 6a | 9 | N.D. |
| 7a | 10 | 20 |
| 8a | 91 | N.D. |
| 9a | 41 | N.D. |
| 10a | 91 | N.D. |
| 11a | 22 | 80 |
| 12a | 23 | 101 |
| 13a | 115 | N.D. |
| 14a | 101 | 318 |
| 15a | 45 | 113 |
| 16a | 182 | N.D. |
| 17a | 182 | N.D. |
| 18a | 51 | N.D. |
| 19a | 51 | N.D. |
| 20a | 182 | N.D. |
| 21a | 102 | N.D. |
| 22a | 36 | N.D. |
| 23a | 16 | N.D. |
| 24a | 41 | N.D. |
| 25a | 36 | N.D. |
| 26a | 23 | N.D. |
| 27a | 18 | N.D. |
| 28a | 204 | N.D. |
| 29a | 106 | N.D. |
| 30a | 576 | N.D. |
| 31a | 162 | N.D. |
| 32a | 408 | N.D. |
| 33a | 65 | N.D. |
| 34a | 51 | N.D. |
| 35a | 26 | N.D. |
| 36a | 41 | N.D. |
| 37a | 32 | N.D. |
| 38a | 51 | N.D. |
| 39a | 32 | N.D. |
| 40a | 115 | N.D. |
| 41a | 289 | N.D. |
| 42a | 363 | N.D. |
| 43a | 23 | N.D. |
| 44a | 51 | N.D. |
| 45a | 15 | 90 |
| 46a | 33 | 252 |
| 47a | 46 | N.D. |
| 48a | 36 | N.D. |
| 49a | 15 | N.D. |
| 50a | 29 | N.D. |
| 51a | 9 | N.D. |
| 52a | 3 | 45 |
| 53a | 1 | 10 |
| 54a | 1 | 7 |
| 55a | 28 | 225 |
| 56a | 8 | 71 |
| 57a | 11 | 90 |
| 58a | 14 | 127 |
| 59a | 7 | 45 |
| 60a | 11 | 90 |
| 61a | 3 | 28 |
| 62a | 1 | 9 |
| 63a | 2 | 13 |
| 64a | 6 | 57 |
| 65a | 3 | 18 |
| 66a | 4 | 28 |
| 67a | 9 | 57 |
| 68a | 3 | 20 |
| 69a | 3 | 25 |
| 71a | 576 | N.D. |
| 72a | 289 | N.D. |
| 73a | 204 | N.D. |
| 74a | 80 | 1005 |
| 75a | 63 | 634 |
| 76a | 101 | 1127 |
| 77a | 16 | 142 |
| 78a | 29 | N.D. |
| 79a | 20 | 101 |
| 80a | 90 | 798 |
| 81a | 90 | 1265 |
| 82a | 127 | 1127 |
| 83a | 16 | 113 |
| 84a | 14 | 113 |
| 85a | 11 | 90 |
| 86a | 18 | 142 |
| 87a | 28 | 252 |
| 88a | 25 | 179 |
| 89a | 18 | 160 |
| 90a | 50 | 449 |
| 91a | 28 | 283 |
| 92a | 5 | 40 |
| 93a | 13 | 142 |
| 94a | 6 | 40 |
| 95a | 113 | 2005 |
| 96a | 16 | 160 |
| 97a | 32 | 318 |
| 98a | 113 | 2524 |
| 99a | 142 | 1419 |
| 100a | 90 | 603 |
| 101a | 113 | 711 |
| 102a | 23 | 71 |
| 103a | 71 | 201 |
| 104a | 711 | 6340 |
| 105a | 711 | 6340 |
| 106a | 11 | 71 |
| 107a | 80 | 1127 |
| 108a | 80 | 798 |
| 109a | 28 | 318 |
| 110a | 18 | 113 |
| 111a | 13 | 113 |
| 112a | 3 | 23 |
| 117a | 159 | 1593 |
| 118a | 179 | 3177 |
| 119a | 4 | 45 |
| 120a | 5 | 45 |
| 121a | 2 | 20 |
| 122a | 11 | 127 |
| 123a | 63 | 1005 |
| 124a | 90 | 1127 |
| 125a | 2 | 16 |
| 126a | 4 | 80 |
| 127a | 8 | 113 |

N.D. = not determined

### Example 38

### Combination Drug Screening for Exemplary Compounds

### THP1 Cells

TABLE 38
Sum excess HSA scores for a combination therapy
of a variety of exemplary compounds with venetoclax
obtained in THP1 cells in a 10 × 10 dataset.

| Com- | Compound | Venetoclax | Excess |
|---|---|---|---|
| pound | Concentration | Concentration | HSA |
| 4a | 0-10 μM | 0-20 μM | −2433.27319 |
| 12a | 0-10 μM | 0-20 μM | −1770.6140 |
| 49a | 0-10 μM | 0-20 μM | −1766.69736 |
| Gilteritinib | 0-20 μM | 0-2 μM | −1626.05178 |
| 60a | 0-0.1 μM | 0-20 μM | −1548.40627 |
| 25a | 0-10 μM | 0-2 μM | −1386.18457 |
| 57a | 0-10 μM | 0-20 μM | −1308.2250 |
| 82a | 0-10 μM | 0-20 μM | −1292.2960 |
| 45a | 0-10 μM | 0-20 μM | −1157.79659 |
| 100a | 0-10 μM | 0-2 μM | −1080.26447 |
| 79a | 0-10 μM | 0-2 μM | −994.90130 |
| 35a | 0-10 μM | 0-20 μM | −937.66386 |
| 59a | 0-10 μM | 0-20 μM | −914.43572 |
| 54a | 0-10 μM | 0-2 μM | −807.18665 |
| 46a | 0-10 μM | 0-2 μM | −774.00715 |
| 48a | 0-10 μM | 0-2 μM | −759.75442 |
| 1a | 0-10 μM | 0-2 μM | −696.55076 |
| 34a | 0-10 μM | 0-20 μM | −683.50774 |
| 92a | 0-10 μM | 0-2 μM | −529.17234 |
| 74a | 0-10 μM | 0-2 μM | −456.14030 |

### MOLM14 (D835Y) Cells

TABLE 39
Sum excess HSA scores for a combination therapy of a
variety of exemplary compounds with venetoclax obtained
in MOLM14 (D835Y) cells in a 10 × 10 dataset.

| Com- | Compound | Venetoclax | Excess |
|---|---|---|---|
| pound | Concentration | Concentration | HSA |
| 45a | 0-0.1 μM | 0-20 μM | −2340.49167 |
| 104a | 0-0.1 μM | 0-20 μM | −2179.06507 |
| 49a | 0-0.05 μM | 0-20 μM | −1959.91073 |
| 59a | 0-0.1 μM | 0-20 μM | −1910.69325 |
| 4a | 0-0.1 μM | 0-20 μM | −1769.02568 |
| 57a | 0-0.1 μM | 0-20 μM | −1762.03659 |
| 48a | 0-0.1 μM | 0-20 μM | −1693.56837 |
| 82a | 0-10 μM | 0-20 μM | −1678.89268 |
| Gilteritinib | 0-0.5 μM | 0-20 μM | −1555.95233 |
| 100a | 0-10 μM | 0-2 μM | −1548.37871 |
| 35a | 0-0.1 μM | 0-20 μM | −1542.77362 |
| 25a | 0-0.1 μM | 0-20 μM | −1507.10714 |
| 1a | 0-0.1 μM | 0-20 μM | −1504.60971 |
| 60a | 0-0.1 μM | 0-20 μM | −1497.3117 |
| 54a | 0-0.1 μM | 0-20 μM | −1490.47075 |
| 34a | 0-10 μM | 0-20 μM | −1437.9420 |
| 74a | 0-10 μM | 0-20 μM | −1334.81247 |
| 46a | 0-0.1 μM | 0-20 μM | −1279.29771 |
| 12a | 0-0.1 μM | 0-20 μM | −1236.04126 |
| 79a | 0-0.1 μM | 0-20 μM | −1180.18758 |

Tables 38 and 39 provide combination data for exemplary compounds of the disclosure with venetoclax in THP1 cells (FLT3 WT) (Table 38) and MOLM14-D835Y cells (FLT3-ITD (D835Y) mutant) (Table 39). These assays were performed using the Cell Titre Glo assay, measured at 48 hours. The compounds were run in 4 different dose combinations (higher and lower concentration range for the compound of the disclosure as well as higher and lower concentration range for venetoclax). The combination that gave the optimal Excess HSA score for synergy (i.e. the largest negative number of the 4 combinations) is shown in the tables along with the dose range of each compound in the optimal combination.

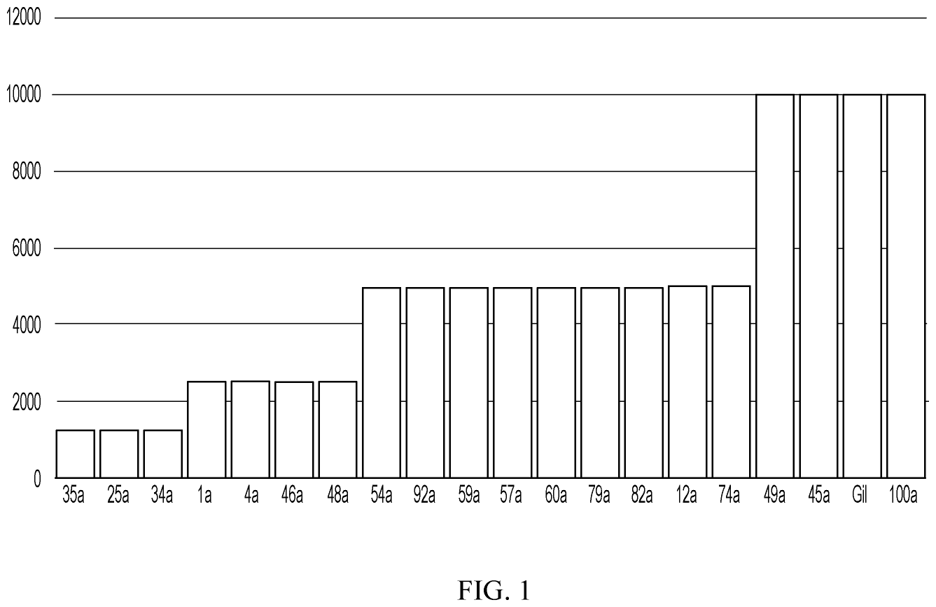

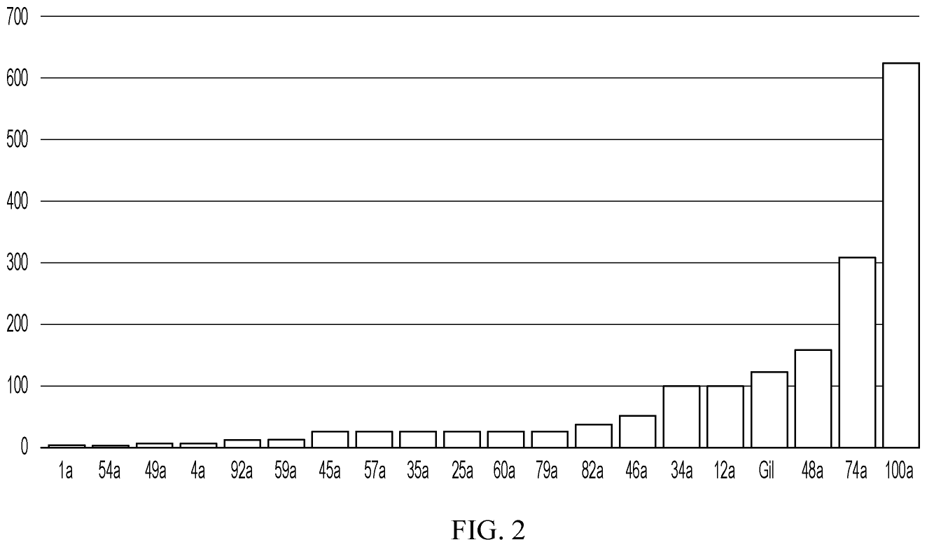

Each of the exemplary compounds in Tables 38 and 39 synergize with venetoclax in both the THP1 and the D835Y cell lines, as demonstrated by the negative Excess Sum HSA values. While some of the Excess Sum HSA values are larger negative numbers than others, the magnitude of the Excess Sum HSA score does not reveal the relative potency of the different drugs to synergize with a given agent. That analysis requires examination of the individual dose-response curve combinations, where it was observed that the compounds of the disclosure are potent in synergizing with Venetoclax (FIG. 1 and FIG. 2).

FIG. 1 and Table 40 depict the combination outcomes for representative compounds of the disclosure with Venetoclax in the Cell Titer Glo assay in THP1 cells at 48 hours. Specifically, FIG. 1 and Table 40 depict the relative concentration (nM) of representative compounds of the disclosure needed to fully potentiate (<20%) of the 312.5 nM Venetoclax Cell Titer Glo response at 48 hours. A smaller concentration indicates higher potency to synergize with Venetoclax.

TABLE 40
Concentration of a compound of the disclosure,
or gilteritinib, needed to decrease the 312.5 nM
Venetoclax response to <20% in THP1 cells.

| Compound # | Concentration (nM) |
|---|---|
| 35a | 1250 |
| 25a | 1250 |
| 34a | 1250 |
| 1a | 2500 |
| 4a | 2500 |
| 46a | 2500 |
| 48a | 2500 |
| 54a | 5000 |
| 92a | 5000 |
| 59a | 5000 |
| 57a | 5000 |
| 60a | 5000 |
| 79a | 5000 |
| 82a | 5000 |
| 12a | 5000 |
| 74a | 5000 |
| 49a | 10000 |
| 45a | 10000 |
| Gilteritinib | 10000 |
| 100a | 10000 |

FIG. 2 and Table 41 depict the combination outcomes for representative compounds of the disclosure with Venetoclax in the Cell Titer Glo assay in MOLM14 (D835Y) cells at 48 hours. Specifically, FIG. 2 and Table 41 depict the relative concentration (nM) of representative compounds of the disclosure needed to fully potentiate (<10%) of the 62.5 nM Venetoclax Cell Titer Glo response at 48 hours. A smaller concentration indicates higher potency to synergize with Venetoclax.

TABLE 41
Concentration of a compound of the disclosure, or
gilteritinib, needed to decrease the 62.5 nM
Ven response to <10% in MOLM14(D835Y) cells.

| Compound # | Concentration (nM) |
|---|---|
| 1a | 1.6 |
| 54a | 1.6 |
| 49a | 6.3 |
| 4a | 6.3 |
| 92a | 12.5 |
| 59a | 12.5 |
| 45a | 25 |
| 57a | 25 |
| 35a | 25 |
| 25a | 25 |
| 60a | 25 |
| 79a | 25 |
| 82a | 39 |
| 46a | 50 |
| 34a | 100 |
| 12a | 100 |
| Gilteritinib | 125 |
| 48a | 156.3 |
| 74a | 312 |
| 100a | 625 |

The relative potency values in FIGS. 1-2 and Tables 40-41 illustrate that the majority of the compounds synergize with Venetoclax at higher potency than does Gilteritinib.

### Example 39

### AML Efficacy Studies in Mice

### Methods

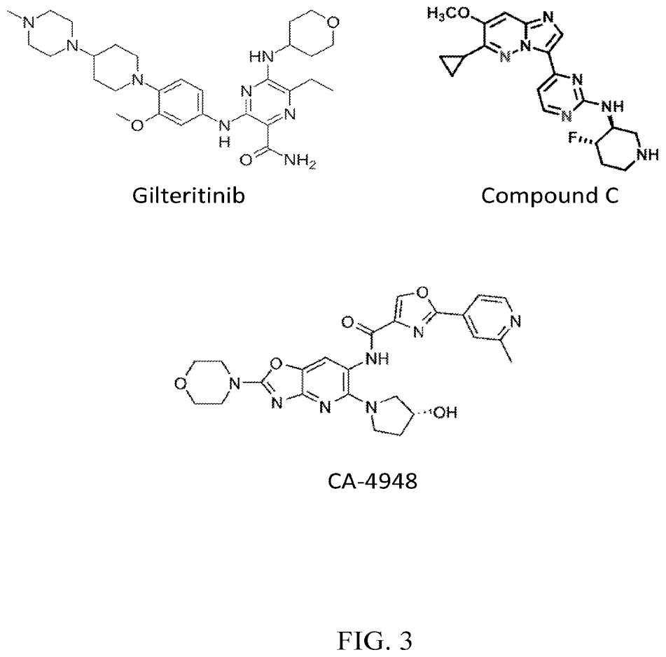

Female, NSG-SGM3 (NOD.Cg-Prkdc^{scid} Il2rg^{tm1Wj1} Tg(CMV-IL3,CSF2,KITLG)1lEav/MloySzJ) mice were intravascularly engrafted with MOLM14-FLT3-ITD(D835Y) acute myeloid leukemia cells. Injection of leukemia cells was performed on 3/21/2022. Mice were randomized and dosing began three weeks following engraftment. Animals were administered vehicle control article or test article daily, Monday through Friday, wherein the compounds used in these studies are depicted in FIG. 3. Mice experiencing clinical signs prior to scheduled end-date, including weight loss, lethargy, hunched posture, or ruffled coat, were humanely euthanized.

Bone marrow aspirates were performed when the initial vehicle control mice became clinical, 9 days following initiation of treatment. Aspirates were centrifuged, treated with RBC lysis buffer, resuspended in 250 L PBS, and two cytospin chamber slides were loaded with 125 μL. Cytospins were used to create two cytology slides per mouse: one for Wright-Giemsa staining and the other for huCD45 immunocytochemistry, if needed. Cytology slides were graded according to the number of leukemic cells identified per 400x field as follows: 0, none; 1, 1-5 CD45+ cells; 2, 6-10 CD45+ cells; 3, 11-15 CD45+ cells; 4, 16-30 CD45+ cells; 5, >30 CD45+ cells.

Mice were submitted for necropsy when they developed clinical signs. Sections of femur, tibia, vertebra, and sternum were evaluated for the presence of leukemic cells using H&E and CD45 immunohistochemistry, if needed. Grades for long bones, vertebrae, and sternum were recorded as follows: 0, no leukemic cells; 1, <1% of cells, 2, 1-10% of cells; 3, 10-50% of bone marrow; 4, >50% of bone marrow. Sums of the individual grades were taken for each mouse (range 0 to 12).

### Results and Discussion

### Survival

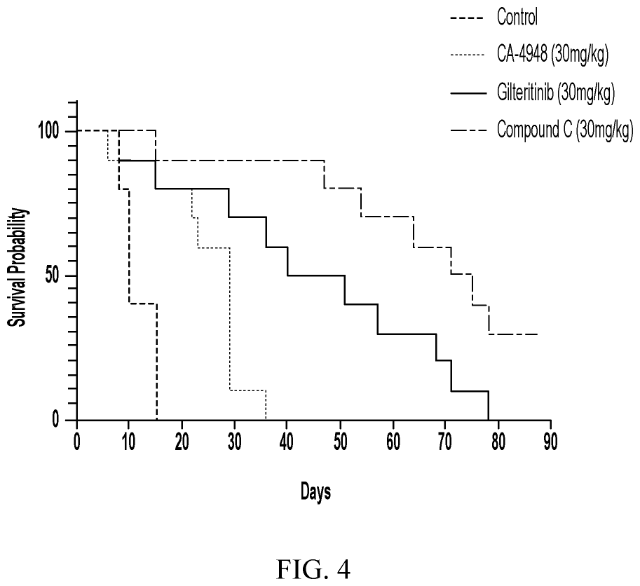

Survival analyses were performed for all groups. 35 mice were censored due to end of study. Significant differences in survival were observed for all treatment groups compared to vehicle control mice (FIG. 4). Significant survival differences are observed for Compound C compared to SOC therapy.

### Bone Marrow Cytology and Hematology for Model Verification and Leukemic Burden Quantification

Leukemic burden was measured in NSGS mice xenografted with MOLM14 FLT3-ITD(D835Y) and quantified using bone marrow (BM) aspirates. An initial BM aspirate, accompanied by peripheral CBC evaluation (submandibular samples), was collected at the time that clinical signs first appeared in control mice. Cytospin chambers were used to create two cytology slides per bone marrow sample. Aspirate samples were of high quality for 85% of samples. All mice had evidence of leukemia within the bone marrow aspirate

### Bone Marrow Histopathology to Quantify Leukemic Burden

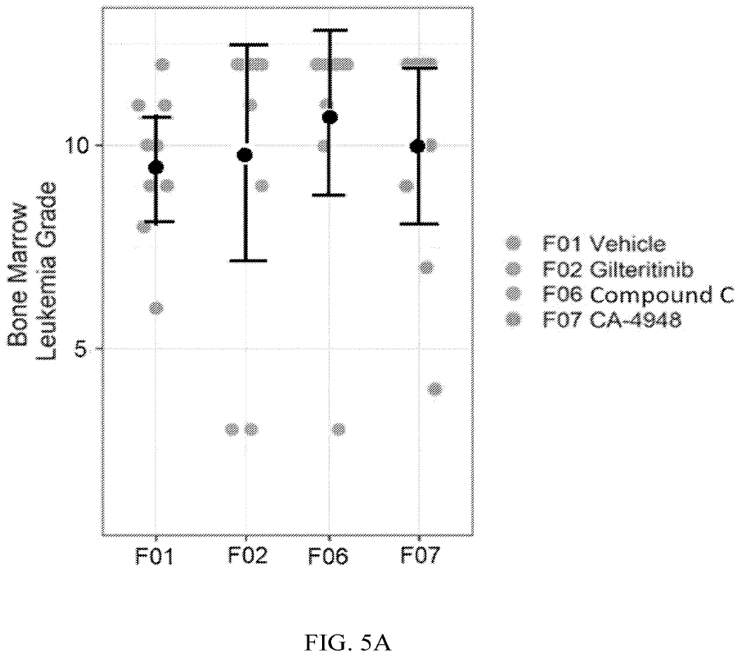

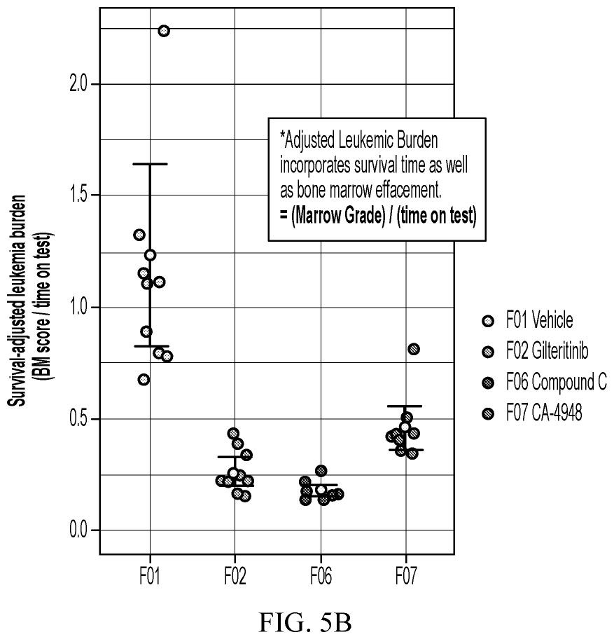

Histopathology scores were taken for long bones (femur and tibia), vertebrae, and sternum as follows: 0, no leukemic cells; 1, <1% of cells; 2, 1-10% of cells; 3, 10-50% of bone marrow; 4, >50% of bone marrow. Scores were totaled (range: 0 to 12) and plotted by group at necropsy (FIG. 5A). To incorporate survival, the bone marrow grade was divided by the time on test (FIG. 5B). When adjusting for survival, all treated animals show significant improvement compared to untreated controls and F06 (Compound C) has decreased scores compared to standard of care (F02).

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements. No language in the specification should be construed as indicating any non-claimed element essential to the practice of the application.

In certain instances, sequences disclosed herein are included in publicly-available databases, such as GENBANK® and SWISSPROT. Unless otherwise indicated or apparent the references to such publicly-available databases are references to the most recent version of the database as of the filing date of this Application.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter. As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the invention. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A compound of formula (I), (II), or (III): 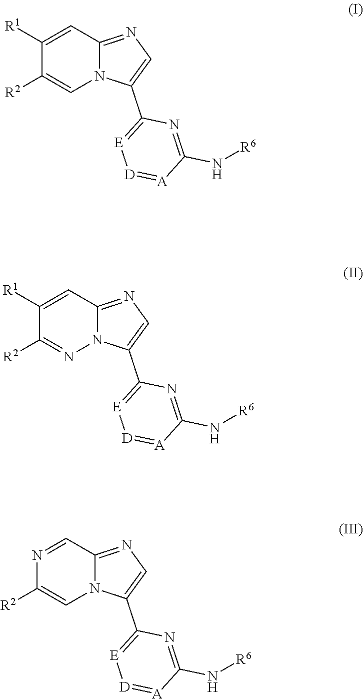
or a salt, ester, solvate, optical isomer, geometric isomer, salt of an isomer, prodrug, or derivative thereof,
wherein:
A is selected from N and CR⁵;
D is selected from N and CR⁴;
E is selected from N and CR³;
at least one of A, D, and E is N;
R¹, R², R³, R⁴, and R⁵ are each independently selected from H, deuterium, halogen, hydroxy, oxo, —CN, —C(═O)H, —C(═O)OH, C₁-C₇ alkyl, C₂-C₇ alkenyl, C₂-C₇ alkynyl, C₁-C₇ alkoxy, —C(═O)NR³¹R³², cycloalkyl, —O-cycloalkyl, spiro-fused cycloalkyl, heterocyclyl, aryl, heteroaryl, or fused ring heteroaryl, wherein —C(═O)H, —C(═)OH, C₁-C₇ alkyl, C₂-C₇ alkenyl, C₂-C₇ alkynyl, C₁-C₇ alkoxy, cycloalkyl, —O-cycloalkyl, spiro-fused cycloalkyl, heterocyclyl, aryl, heteroaryl, or fused ring heteroaryl is optionally substituted with one or more of deuterium, halogen, hydroxy, oxo, —C(═O)H, —C(═O)OH, nitro (—NO₂), —NH₂, —N(CH₃)₂, cyano (—CN), ethynyl (—CCH), propynyl, —SO₃H, cycloalkyl, heterocyclyl, aryl, heteroaryl, pyrrolyl, piperidyl, piperazinyl, morpholinyl, —C(═O)-morpholin-4-yl, —C(═O)NH₂, —C(═O)N(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl;
R⁶is 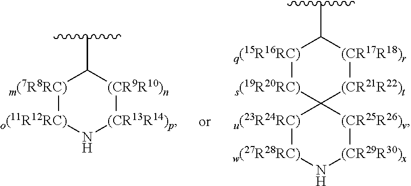
or C₃-C₆ cycloalkyl substituted with one or more —NR³³R³⁴;
R⁷, R⁸, R⁹, R¹⁰, R¹¹, R¹², R¹³, and R¹⁴ are each independently selected from H, deuterium, halogen, hydroxy, oxo, —CN, —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more halogen;
R¹⁵, R¹⁶, R¹⁷, R¹⁸, R¹⁹, R²⁰, R²¹, R²², R²³, R²⁴, R²⁵, R²⁶, R²⁷, R²⁹, R²⁹, and R³⁰ are independently selected from H, deuterium, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more halogen;
R³¹ and R³² are each independently selected from H, C₁-C₆ alkyl, and C₃-C₆ cycloalkyl, wherein C₁-C₆ alkyl and C₃-C₆ cycloalkyl are optionally substituted with one or more halogen;
R³³ and R³⁴ are each independently selected from H and C₁-C₆ alkyl; and
m, n, o, p, q, r, s, t, u, v, w, and x are independently selected from 0, 1, 2, 3, 4, or 5, where q+r+s+t is at least 1, and where u+v+w+x is at least 1.

2. The compound of claim 1, wherein the compound of Formula (I) is a compound of Formula (Ib-5010-WO50): 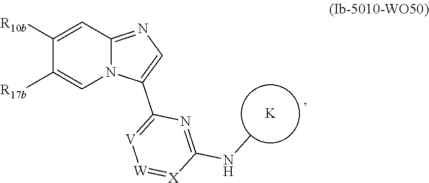
or a salt, ester, solvate, optical isomer, geometric isomer, or salt of an isomer thereof;
wherein:
V is N or CR₁₁;
W is N or CR₁₂;
X is N or CR₁₃; 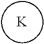
is 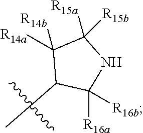
R_{10b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from C₁-C₆ alkyl and halogen;
R_{17b} is selected from halogen, C₁-C₆ alkyl, C₁-C₆ alkoxy, C₃-C₆ cycloalkyl, —O—(C₃-C₆ cycloalkyl), C₃-C₉ heterocyclyl, imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from —OH, C₁-C₆ alkyl, and halogen;
R₁₁, R₁₂, and R₁₃ are each independently selected from H, C₁-C₆ alkoxy, and halogen;
R₁₄ₐ, R_{14b}, R₁₅ₐ, R_{15b}, R₁₆ₐ, R_{16b}, R₁₈ₐ, and R_{18b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of V, W, or X is N.

3. The compound of claim 2, wherein at least one of (i)-(v) applies:
(i) each of R_{14b}, R₁₅ₐ, R_{15b}, R₁₆ₐ, and R_{16b} is H and R₁₄ₐ is F;
(ii) R₁₁, R₁₂, and R₁₃, if present, are H;
(iii) V is CR₁₁ wherein R₁₁ is F, W is CR₁₂ wherein R₁₂ is H, and X is N;
(iv) R_{10b} is selected from H and —OCH₃; and
(v) R_{17b} is selected from

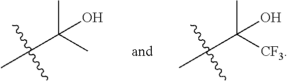

4. The compound of claim 2 or 3, with the provisos that:
when R_{17b} is 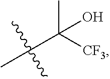 [SMILES: *C(C)(O)C(F)(F)F]
R_{10b} is H; and
when R_{17b} is 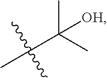 [SMILES: *C(C)(C)O]
at least one of R₁₁, R₁₂, and R₁₃ is present and selected from C₁-C₆ alkoxy and halogen.

5. The compound of any one of claims 2 to 4, wherein the compound of Formula (Ib-5010-WO50) is selected from:

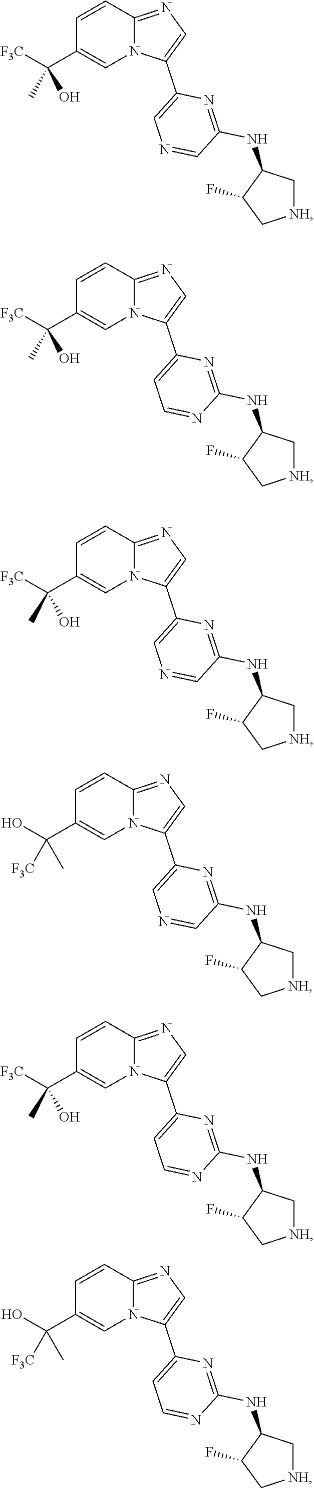 [SMILES: C[C@@](O)(c1ccc2ncc(-c3cncc(N[C@H]4CNC[C@@H]4F)n3)n2c1)C(F)(F)F ; C[C@@](O)(c1ccc2ncc(-c3ccnc(N[C@H]4CNC[C@@H]4F)n3)n2c1)C(F)(F)F ; C[C@](O)(c1ccc2ncc(-c3cncc(N[C@H]4CNC[C@@H]4F)n3)n2c1)C(F)(F)F ; CC(O)(c1ccc2ncc(-c3cncc(N[C@H]4CNC[C@@H]4F)n3)n2c1)C(F)(F)F ; C[C@](O)(c1ccc2ncc(-c3ccnc(N[C@H]4CNC[C@@H]4F)n3)n2c1)C(F)(F)F ; CC(O)(c1ccc2ncc(-c3ccnc(N[C@H]4CNC[C@@H]4F)n3)n2c1)C(F)(F)F]

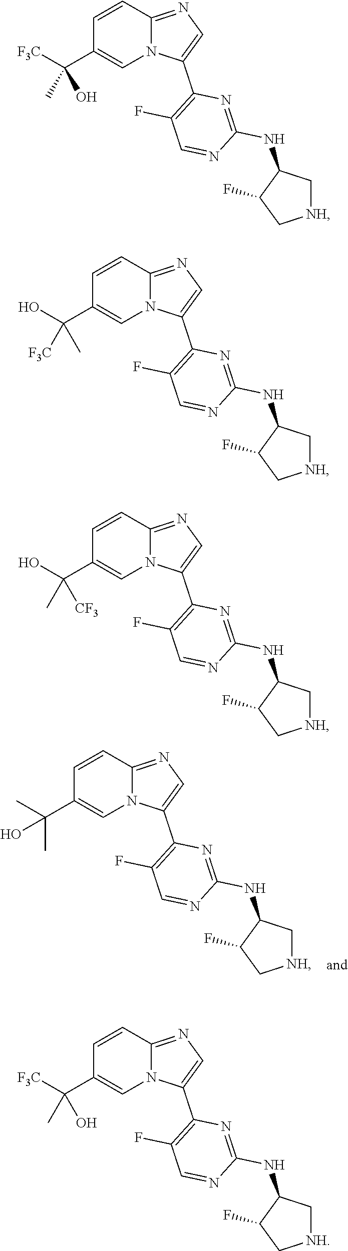 [SMILES: C[C@@](O)(c1ccc2ncc(-c3nc(N[C@H]4CNC[C@@H]4F)ncc3F)n2c1)C(F)(F)F ; CC(O)(c1ccc2ncc(-c3nc(N[C@H]4CNC[C@@H]4F)ncc3F)n2c1)C(F)(F)F ; CC(O)(c1ccc2ncc(-c3nc(N[C@H]4CNC[C@@H]4F)ncc3F)n2c1)C(F)(F)F ; CC(C)(O)c1ccc2ncc(-c3nc(N[C@H]4CNC[C@@H]4F)ncc3F)n2c1 ; CC(O)(c1ccc2ncc(-c3nc(N[C@H]4CNC[C@@H]4F)ncc3F)n2c1)C(F)(F)F]

6. The compound of claim 1, wherein the compound of Formula (I) is a compound of Formula (Id-5010-WO50): 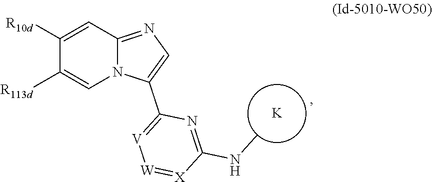
or a salt, ester, solvate, optical isomer, geometric isomer, or salt of an isomer thereof;
wherein:
V is N or CR₁₁;
W is N or CR₁₂;
X is N or CR₁₃; 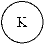
is 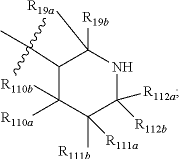
R_{10d} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and halogen, and C₃-C₆ cycloalkyl and wherein —O—(C₃-C₆ cycloalkyl) are each optionally substituted with one or more substituents selected from C₁-C₆ alkyl and halogen;
R_{113d} is selected from halogen, C₁-C₆ alkyl, C₁-C₆alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from —OH, C₁-C₆ alkyl, and halogen;
R₁₁, R₁₂, and R₁₃ are each independently selected from H, C₁-C₆ alkoxy, and halogen;
R₁₈ₐ and R_{18b} are each independently selected from H, C₁-C₆ alkyl, and C₃-C₆ cycloalkyl, wherein C₁-C₆ alkyl and C₃-C₆ cycloalkyl are each optionally substituted with one or more halogen;
R₁₉ₐ, R_{19b}, R₁₁₀ₐ, R_{110b}, R₁₁₁ₐ, R_{111b}, R₁₁₂ₐ, and R_{112b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of V, W, or X is N.

7. The compound of claim 6, wherein at least one of (i)-(vi) applies:
(i) each of R₉ₐ, R_{19b}, R₁₁₀ₐ, R_{110b}, R₁₁₁ₐ, R_{111b}, R₁₁₂ₐ, and R_{112b} is H;
(ii) each of R₁₉ₐ, R_{19b}, R_{110b}, R₁₁₁ₐ, R_{111b}, R₁₁₂ₐ, and R_{112b} is H and R₁₁₀ₐ is F;
(iii) R₁₁, R₁₂, and R₁₃, if present, are H;
(iv) V is CR₁₁ wherein R₁₁ is F, W is CR₁₂ wherein R₁₂ is H, and X is N;
(v) R_{10d} is selected from H and —OCH₃; and
(vi) R_{113d} is selected from

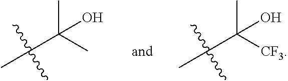

8. The compound of claim 6 or 7, with the provisos that:
when R_{113d} is 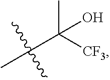 [SMILES: *C(C)(O)C(F)(F)F]
R_{10d} is H; and
when R_{113d} is 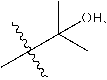 [SMILES: *C(C)(C)O]
at least one of R₁₁, R₁₂, and R₁₃ is present and selected from C₁-C₆ alkoxy and halogen.

9. The compound of any one of claims 6 to 8 wherein the compound of Formula (Id-5010-WO50) is selected from:

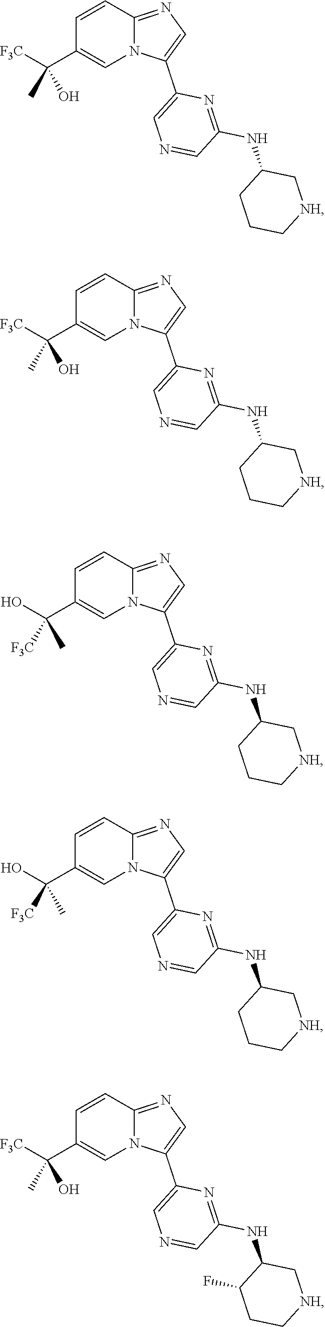 [SMILES: C[C@](O)(c1ccc2ncc(-c3cncc(N[C@H]4CCCNC4)n3)n2c1)C(F)(F)F ; C[C@@](O)(c1ccc2ncc(-c3cncc(N[C@H]4CCCNC4)n3)n2c1)C(F)(F)F ; C[C@@](O)(c1ccc2ncc(-c3cncc(N[C@@H]4CCCNC4)n3)n2c1)C(F)(F)F ; C[C@](O)(c1ccc2ncc(-c3cncc(N[C@@H]4CCCNC4)n3)n2c1)C(F)(F)F ; C[C@@](O)(c1ccc2ncc(-c3cncc(N[C@H]4CNCC[C@@H]4F)n3)n2c1)C(F)(F)F]

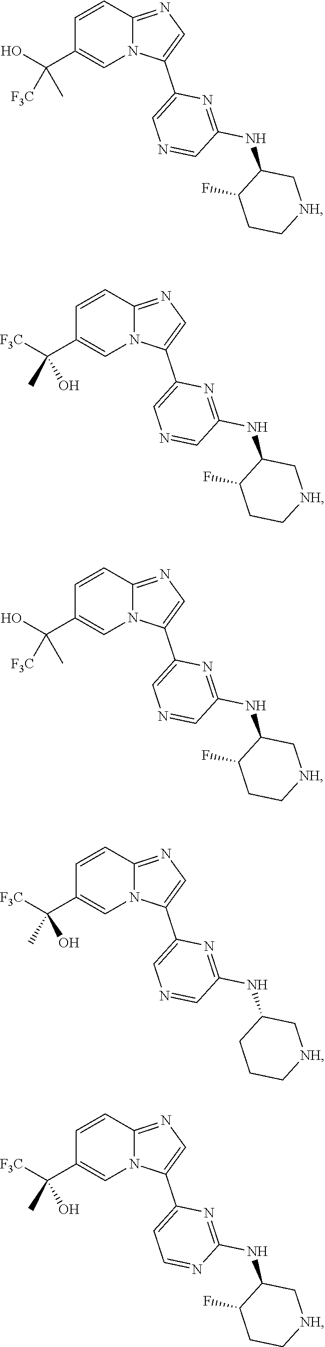 [SMILES: CC(O)(c1ccc2ncc(-c3cncc(N[C@H]4CNCC[C@@H]4F)n3)n2c1)C(F)(F)F ; C[C@](O)(c1ccc2ncc(-c3cncc(N[C@H]4CNCC[C@@H]4F)n3)n2c1)C(F)(F)F ; CC(O)(c1ccc2ncc(-c3cncc(N[C@H]4CNCC[C@@H]4F)n3)n2c1)C(F)(F)F ; C[C@@](O)(c1ccc2ncc(-c3cncc(N[C@H]4CCCNC4)n3)n2c1)C(F)(F)F ; C[C@](O)(c1ccc2ncc(-c3ccnc(N[C@H]4CNCC[C@@H]4F)n3)n2c1)C(F)(F)F]

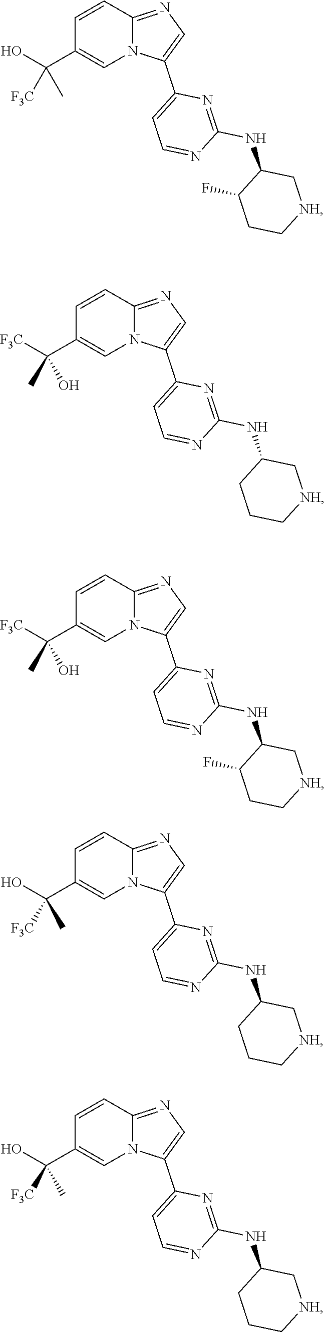 [SMILES: CC(O)(c1ccc2ncc(-c3ccnc(N[C@H]4CNCC[C@@H]4F)n3)n2c1)C(F)(F)F ; C[C@](O)(c1ccc2ncc(-c3ccnc(N[C@H]4CCCNC4)n3)n2c1)C(F)(F)F ; C[C@](O)(c1ccc2ncc(-c3ccnc(N[C@H]4CNCC[C@@H]4F)n3)n2c1)C(F)(F)F ; C[C@@](O)(c1ccc2ncc(-c3ccnc(N[C@@H]4CCCNC4)n3)n2c1)C(F)(F)F ; C[C@](O)(c1ccc2ncc(-c3ccnc(N[C@@H]4CCCNC4)n3)n2c1)C(F)(F)F]

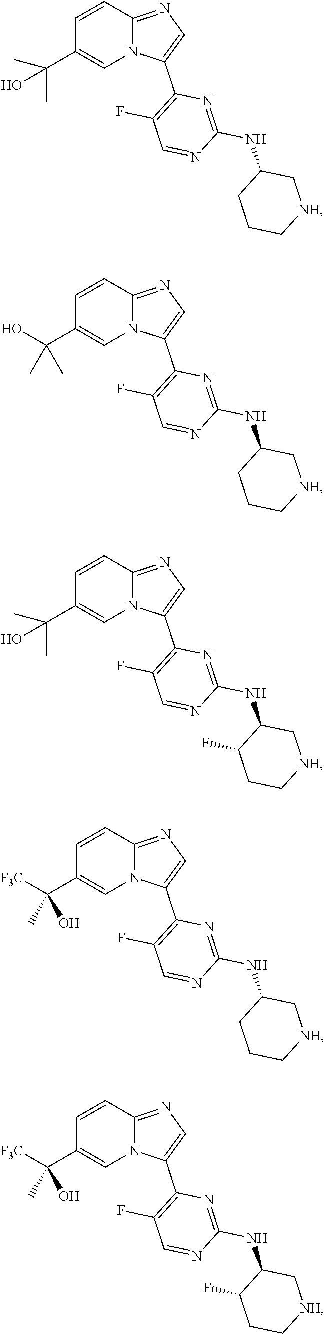 [SMILES: CC(C)(O)c1ccc2ncc(-c3nc(N[C@H]4CCCNC4)ncc3F)n2c1 ; CC(C)(O)c1ccc2ncc(-c3nc(N[C@@H]4CCCNC4)ncc3F)n2c1 ; CC(C)(O)c1ccc2ncc(-c3nc(N[C@H]4CNCC[C@@H]4F)ncc3F)n2c1 ; C[C@@](O)(c1ccc2ncc(-c3nc(N[C@H]4CCCNC4)ncc3F)n2c1)C(F)(F)F ; C[C@@](O)(c1ccc2ncc(-c3nc(N[C@H]4CNCC[C@@H]4F)ncc3F)n2c1)C(F)(F)F]

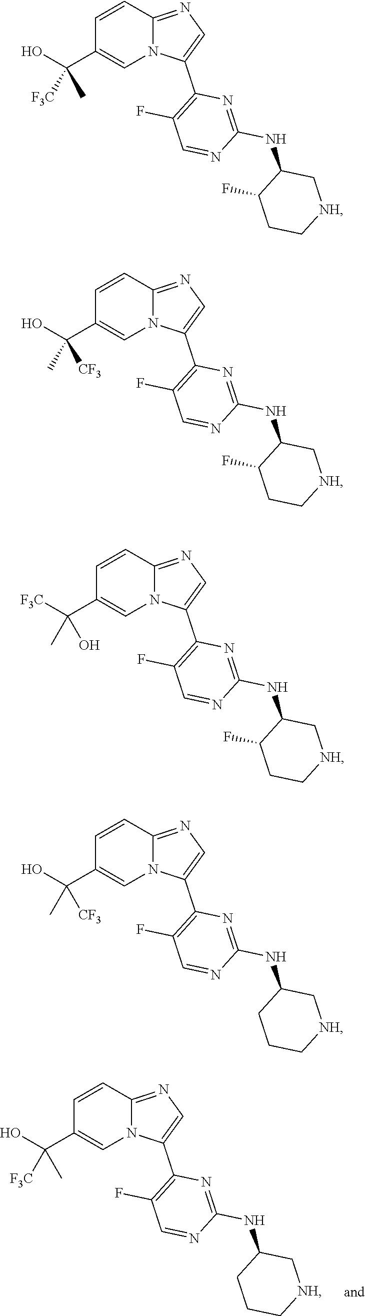 [SMILES: C[C@@](O)(c1ccc2ncc(-c3nc(N[C@H]4CNCC[C@@H]4F)ncc3F)n2c1)C(F)(F)F ; C[C@](O)(c1ccc2ncc(-c3nc(N[C@H]4CNCC[C@@H]4F)ncc3F)n2c1)C(F)(F)F ; CC(O)(c1ccc2ncc(-c3nc(N[C@H]4CNCC[C@@H]4F)ncc3F)n2c1)C(F)(F)F ; CC(O)(c1ccc2ncc(-c3nc(N[C@@H]4CCCNC4)ncc3F)n2c1)C(F)(F)F ; CC(O)(c1ccc2ncc(-c3nc(N[C@@H]4CCCNC4)ncc3F)n2c1)C(F)(F)F]

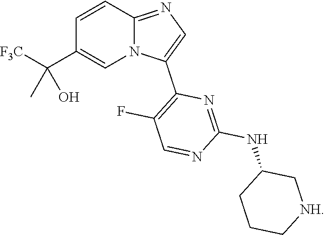 [SMILES: CC(O)(c1ccc2ncc(-c3nc(N[C@H]4CCCNC4)ncc3F)n2c1)C(F)(F)F]

10. The compound of claim 1, wherein the compound of Formula (II) is a compound of Formula (IIa-5010-WO50): 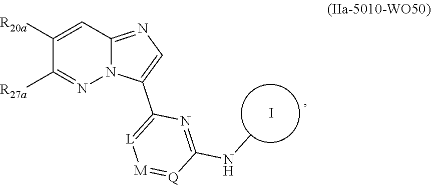
or a salt, ester, solvate, optical isomer, geometric isomer, or salt of an isomer thereof;
wherein:
L is N or CR₂₁;
M is N or CR₂₂;
Q is N or CR₂₃; 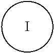
is 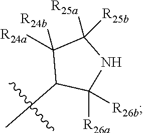
R₂₀ₐ is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from C₁-C₆ alkyl and halogen;
R₂₇ₐ is selected from halogen, C₁-C₆ alkyl, C₁-C₆ alkoxy, C₃-C₆ cycloalkyl, spiro-fused cycloalkyl, —O—(C₃-C₆ cycloalkyl), C₂-C₆ heterocyclyl, imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl, spiro-cycloalkyl, —O—(C₃-C₆ cycloalkyl), and C₂-C₆ heterocyclyl are each optionally substituted with one or more substituents selected from —OH, C₁-C₆ alkyl, and halogen;
R₂₁, R₂₂, and R₂₃ are each independently selected from H, C₁-C₆ alkoxy, and halogen;
R₂₄ₐ, R_{24b}, R₂₅ₐ, R_{25b}, R₂₆ₐ, and R_{26b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms;
R₂₈ₐ and R_{28b} are each independently selected from H, C₁-C₆ alkyl, and C₃-C₆ cycloalkyl, wherein C₁-C₆ alkyl and C₃-C₆ cycloalkyl are each optionally substituted with one or more halogen; and
one of L, M, or Q is N.

11. The compound of claim 10, wherein at least one of (i)-(v) applies:
(i) each of R_{24b}, R₂₅ₐ, R_{25b}, R₂₆ₐ, and R_{26b} is H and R₂₄ₐ is F;
(ii) R₂₁, R₂₂, and R₂₃, if present, are H;
(iii) L is CR₂₁ wherein R₂₁ is selected from F, Cl, and —OCH₃, M is CR₂₂ wherein R₂₂ is H, and Q is N;
(iv) R₂₀ₐ is selected from —OCH₃, unsubstituted —O—(C₃ cycloalkyl), and 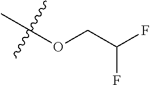 [SMILES: *OCC(F)F]
and
(v) R₂₇ₐ is selected from unsubstituted C₃-C₆ cycloalkyl,

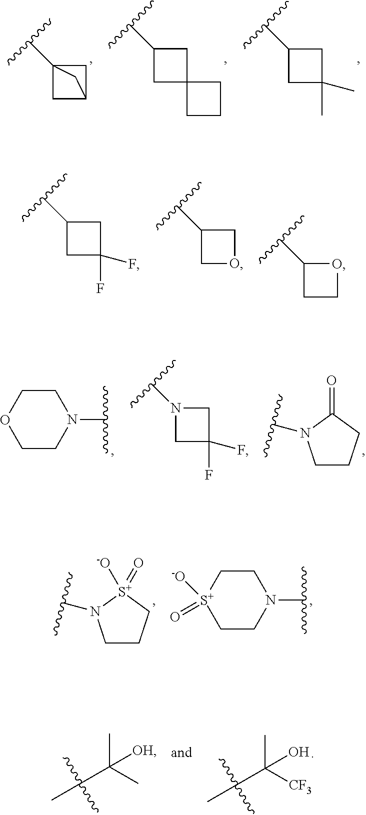

12. The compound of claim 10 or 11, with the proviso that:
when R₂₀ₐ is —OCH₃ and R₂₇ₐ is unsubstituted C₃ cycloalkyl, at least one of R₂₁, R₂₂, and R₂₃ is present and selected from C₁-C₆ alkoxy and halogen.

13. The compound of any one of claims 10 to 12, wherein the compound of Formula (IIa-5010-WO50) is selected from:

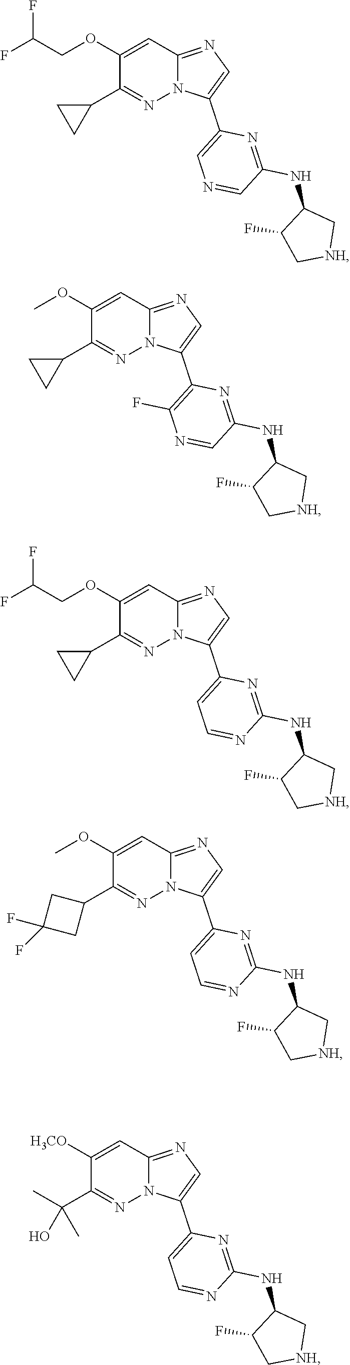 [SMILES: FC(F)COc1cc2ncc(-c3cncc(N[C@H]4CNC[C@@H]4F)n3)n2nc1C1CC1 ; COc1cc2ncc(-c3nc(N[C@H]4CNC[C@@H]4F)cnc3F)n2nc1C1CC1 ; FC(F)COc1cc2ncc(-c3ccnc(N[C@H]4CNC[C@@H]4F)n3)n2nc1C1CC1 ; COc1cc2ncc(-c3ccnc(N[C@H]4CNC[C@@H]4F)n3)n2nc1C1CC(F)(F)C1 ; COc1cc2ncc(-c3ccnc(N[C@H]4CNC[C@@H]4F)n3)n2nc1C(C)(C)O]

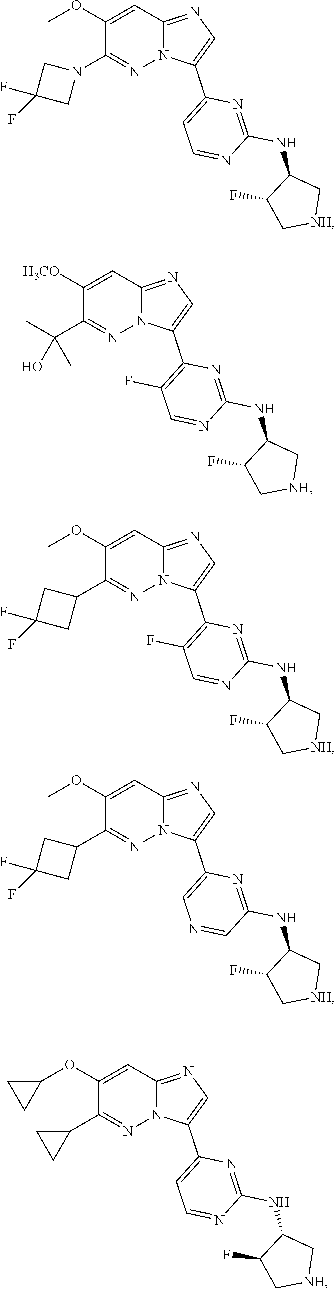 [SMILES: COc1cc2ncc(-c3ccnc(N[C@H]4CNC[C@@H]4F)n3)n2nc1N1CC(F)(F)C1 ; COc1cc2ncc(-c3nc(N[C@H]4CNC[C@@H]4F)ncc3F)n2nc1C(C)(C)O ; COc1cc2ncc(-c3nc(N[C@H]4CNC[C@@H]4F)ncc3F)n2nc1C1CC(F)(F)C1 ; COc1cc2ncc(-c3cncc(N[C@H]4CNC[C@@H]4F)n3)n2nc1C1CC(F)(F)C1 ; F[C@@H]1CNC[C@H]1Nc1nccc(-c2cnc3cc(OC4CC4)c(C4CC4)nn23)n1]

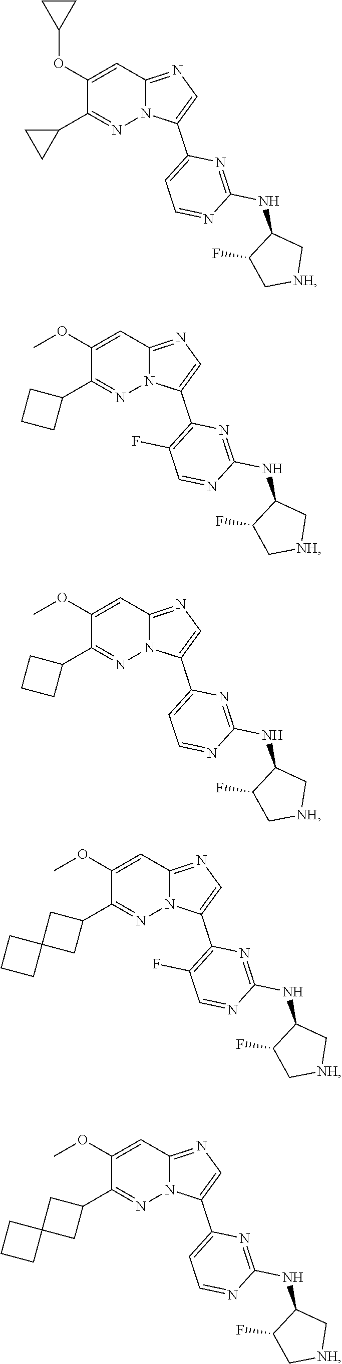 [SMILES: F[C@H]1CNC[C@@H]1Nc1nccc(-c2cnc3cc(OC4CC4)c(C4CC4)nn23)n1 ; COc1cc2ncc(-c3nc(N[C@H]4CNC[C@@H]4F)ncc3F)n2nc1C1CCC1 ; COc1cc2ncc(-c3ccnc(N[C@H]4CNC[C@@H]4F)n3)n2nc1C1CCC1 ; COc1cc2ncc(-c3nc(N[C@H]4CNC[C@@H]4F)ncc3F)n2nc1C1CC2(CCC2)C1 ; COc1cc2ncc(-c3ccnc(N[C@H]4CNC[C@@H]4F)n3)n2nc1C1CC2(CCC2)C1]

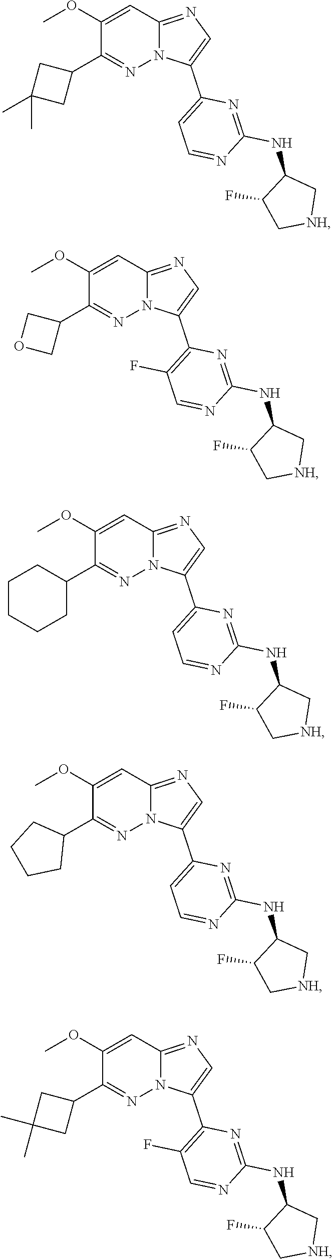 [SMILES: COc1cc2ncc(-c3ccnc(N[C@H]4CNC[C@@H]4F)n3)n2nc1C1CC(C)(C)C1 ; COc1cc2ncc(-c3nc(N[C@H]4CNC[C@@H]4F)ncc3F)n2nc1C1COC1 ; COc1cc2ncc(-c3ccnc(N[C@H]4CNC[C@@H]4F)n3)n2nc1C1CCCCC1 ; COc1cc2ncc(-c3ccnc(N[C@H]4CNC[C@@H]4F)n3)n2nc1C1CCCC1 ; COc1cc2ncc(-c3nc(N[C@H]4CNC[C@@H]4F)ncc3F)n2nc1C1CC(C)(C)C1]

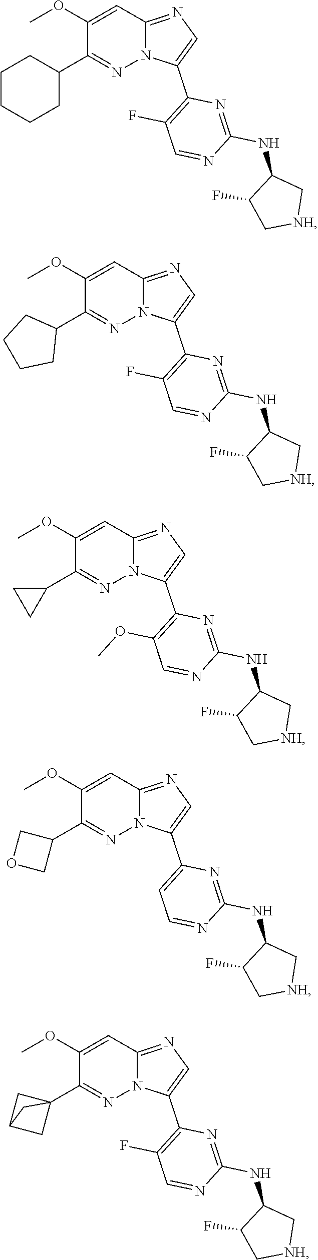 [SMILES: COc1cc2ncc(-c3nc(N[C@H]4CNC[C@@H]4F)ncc3F)n2nc1C1CCCCC1 ; COc1cc2ncc(-c3nc(N[C@H]4CNC[C@@H]4F)ncc3F)n2nc1C1CCCC1 ; COc1cnc(N[C@H]2CNC[C@@H]2F)nc1-c1cnc2cc(OC)c(C3CC3)nn12 ; COc1cc2ncc(-c3ccnc(N[C@H]4CNC[C@@H]4F)n3)n2nc1C1COC1 ; COc1cc2ncc(-c3nc(N[C@H]4CNC[C@@H]4F)ncc3F)n2nc1C12CC(C1)C2]

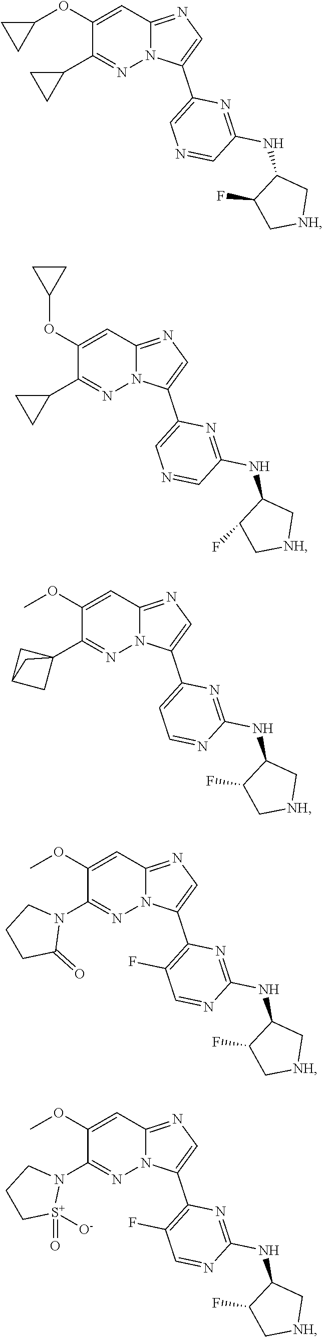 [SMILES: F[C@@H]1CNC[C@H]1Nc1cncc(-c2cnc3cc(OC4CC4)c(C4CC4)nn23)n1 ; F[C@H]1CNC[C@@H]1Nc1cncc(-c2cnc3cc(OC4CC4)c(C4CC4)nn23)n1 ; COc1cc2ncc(-c3ccnc(N[C@H]4CNC[C@@H]4F)n3)n2nc1C12CC(C1)C2 ; COc1cc2ncc(-c3nc(N[C@H]4CNC[C@@H]4F)ncc3F)n2nc1N1CCCC1=O ; COc1cc2ncc(-c3nc(N[C@H]4CNC[C@@H]4F)ncc3F)n2nc1N1CCC[S+]1(=O)[O-]]

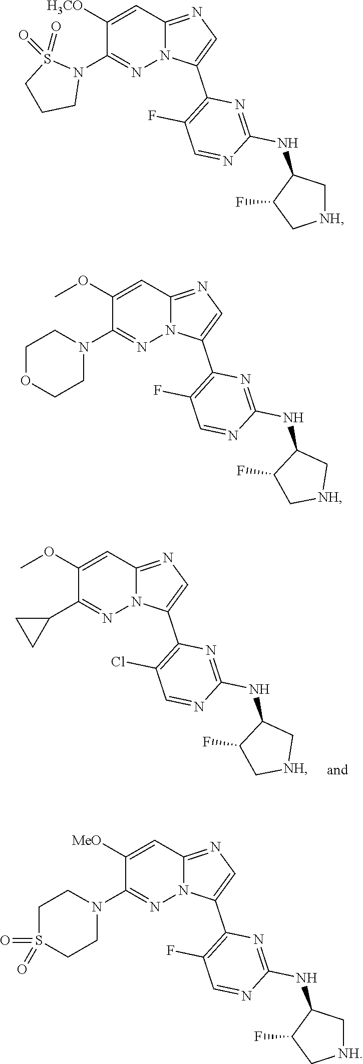 [SMILES: COc1cc2ncc(-c3nc(N[C@H]4CNC[C@@H]4F)ncc3F)n2nc1N1CCCS1(=O)=O ; COc1cc2ncc(-c3nc(N[C@H]4CNC[C@@H]4F)ncc3F)n2nc1N1CCOCC1 ; COc1cc2ncc(-c3nc(N[C@H]4CNC[C@@H]4F)ncc3Cl)n2nc1C1CC1 ; COc1cc2ncc(-c3nc(N[C@H]4CNC[C@@H]4F)ncc3F)n2nc1N1CCS(=O)(=O)CC1]

14. The compound of claim 1, wherein the compound of Formula (II) is a compound of Formula (IIb-5010-WO50): 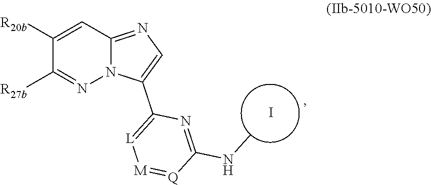
or a salt, ester, solvate, optical isomer, geometric isomer, or salt of an isomer thereof;
wherein:
L is N or CR₂₁;
M is N or CR₂₂;
Q is N or CR₂₃; 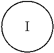
is 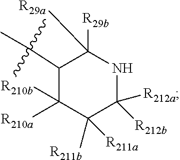
R_{20b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl and —O—(C₃-C₆ cycloalkyl) are each optionally substituted with one or more substituents selected from C₁-C₆ alkyl and halogen;
R_{27b} is selected from halogen, C₁-C₆ alkyl, C₁-C₆ alkoxy, C₃-C₆ cycloalkyl, spiro-cycloalkyl, —O—(C₃-C₆ cycloalkyl), C₂-C₆ heterocyclyl, imidazolyl, triazolyl, and —C(═O)NR₂₈ₐR_{28b}, wherein C₁-C₆ alkyl and C₁-C₆ alkoxy are each optionally substituted with one or more substituents selected from —OH, deuterium, and halogen, and wherein C₃-C₆ cycloalkyl, spiro-cycloalkyl, —O—(C₃-C₆ cycloalkyl), and C₂-C₆ heterocyclyl are each optionally substituted with one or more substituents selected from —OH, C₁-C₆ alkyl, and halogen;
R₂₁, R₂₂, and R₂₃ are each independently selected from H, C₁-C₆ alkoxy, and halogen;
R₂₉ₐ, R_{29b}, R₂₁₀ₐ, R_{210b}, R₂₁₁ₐ, R_{211b}, R₂₁₂ₐ, and R_{212b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms;
R₂₈ₐ and R_{28b} are each independently selected from H, C₁-C₆ alkyl, and C₃-C₆ cycloalkyl, wherein C₁-C₆ alkyl and C₃-C₆ cycloalkyl are each optionally substituted with one or more halogen; and
one of L, M, or Q is N.

15. The compound of claim 14, wherein at least one of (i)-(vi) applies:
(i) each of R₂₉ₐ, R_{29b}, R₂₁₀ₐ, R_{210b}, R₂₁₁ₐ, R_{211b}, R₂₁₂ₐ, and R_{212b} is H;
(ii) each of R₂₉ₐ, R_{29b}, R_{210b}, R₂₁₁ₐ, R_{211b}, R₂₁₂ₐ, and R_{212b} is H and R₂₁₀ₐ is F;
(iii) R₂₁, R₂₂, and R₂₃, if present, are H;
(iv) L is CR₂₁ wherein R₂₁ is selected from F, Cl, and —OCH₃, M is CR₂₂ wherein R₂₂ is H, and Q is N;
(v) R_{20b} is selected from —OCH₃, —OCD₃, unsubstituted —O—(C₃ cycloalkyl), and 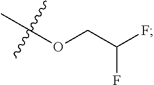 [SMILES: *OCC(F)F]
and
(vi) R_{27b} is selected from unsubstituted C₃-C₆ cycloalkyl,

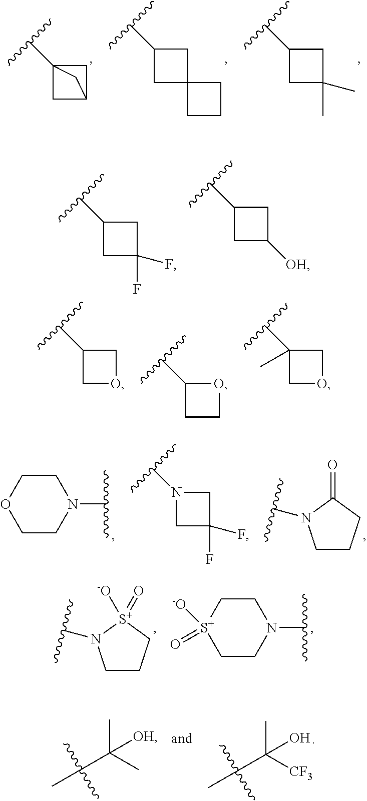

16. The compound of claim 14 or 15, with the proviso that:
when R_{20b} is —OCH₃ and R_{27b} is unsubstituted C₃ cycloalkyl, at least one of R₂₁, R₂₂, and R₂₃ is present and selected from C₁-C₆ alkoxy and halogen.

17. The compound of any one of claims 14 to 16, wherein the compound of Formula (IIb-5010-WO50) is selected from:

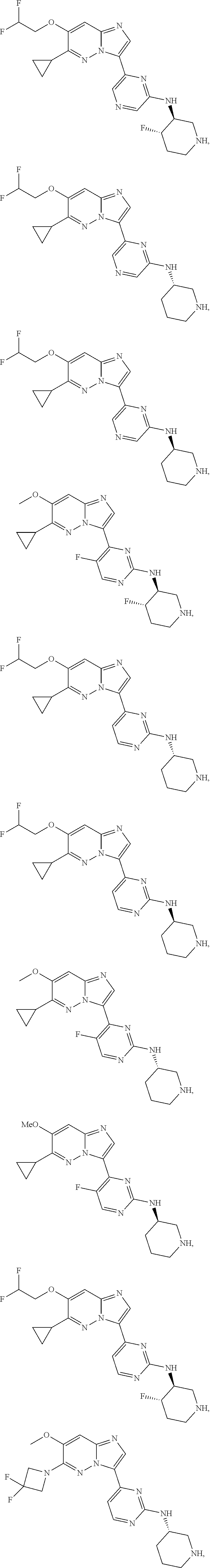

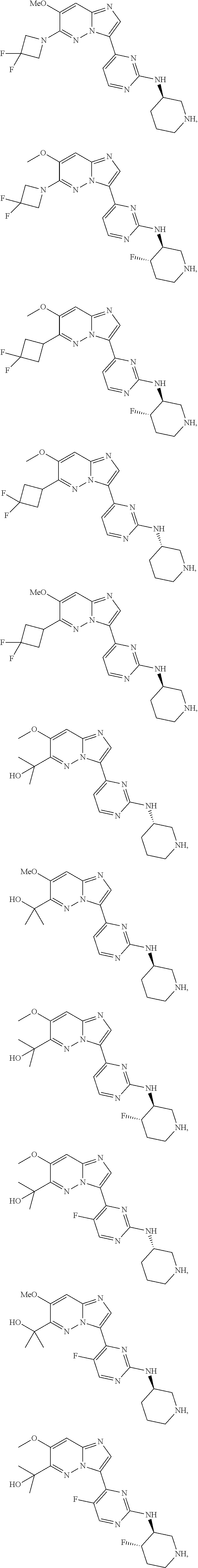

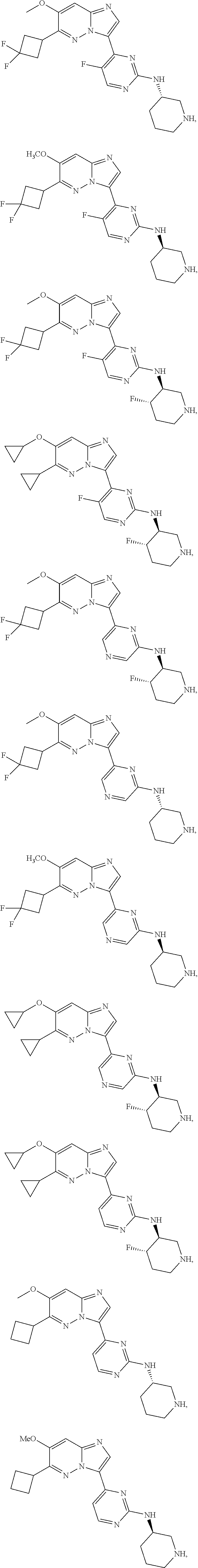

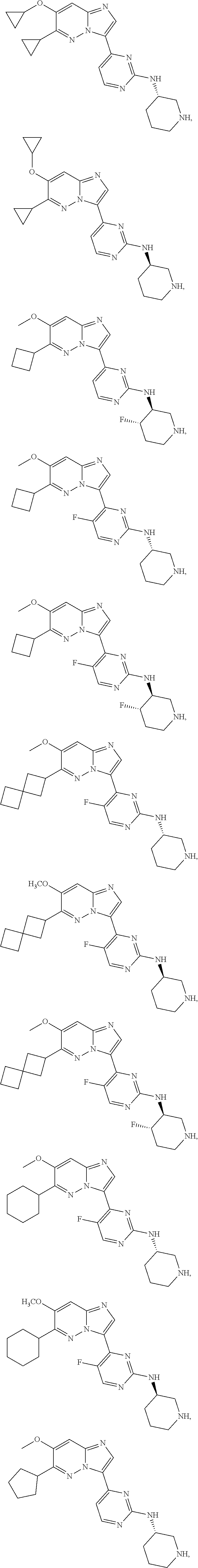

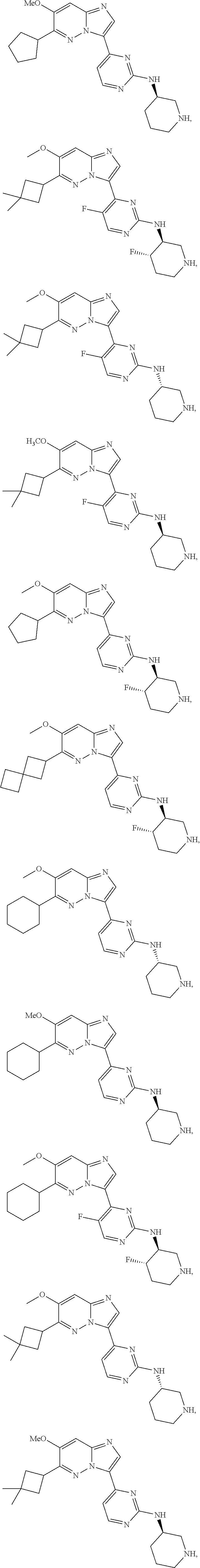

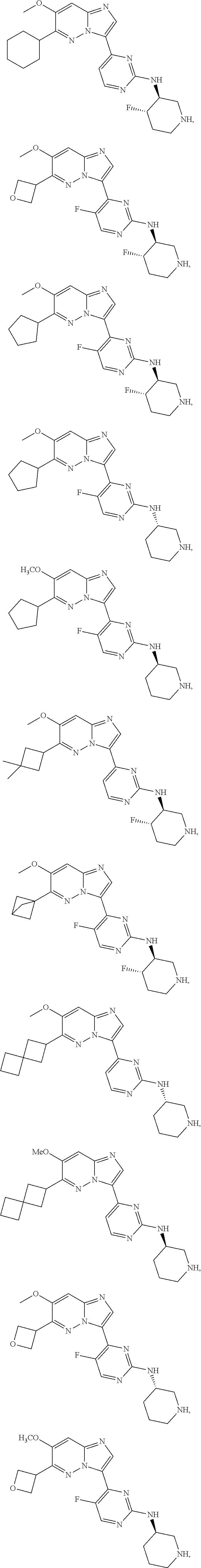

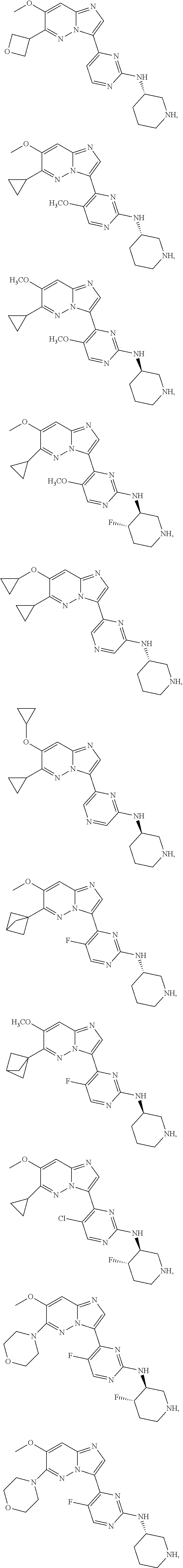

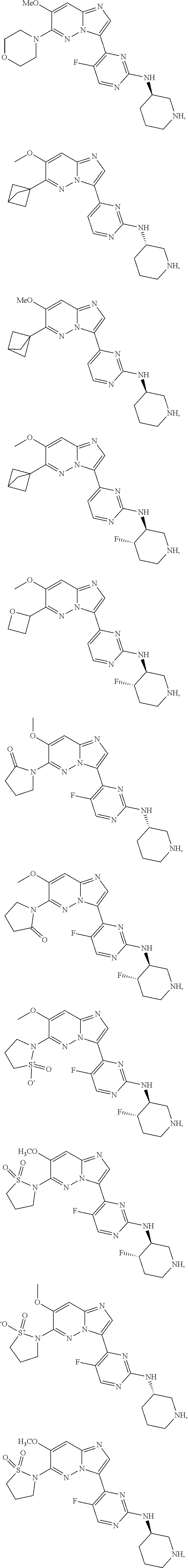

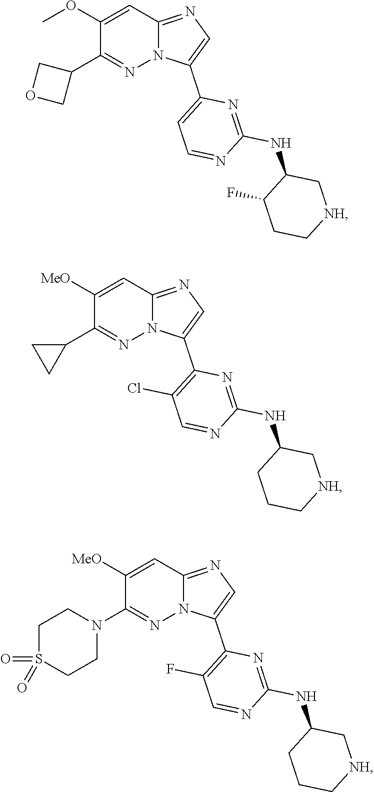 [SMILES: COc1cc2ncc(-c3ccnc(N[C@H]4CNCC[C@@H]4F)n3)n2nc1C1COC1 ; COc1cc2ncc(-c3nc(N[C@@H]4CCCNC4)ncc3Cl)n2nc1C1CC1 ; COc1cc2ncc(-c3nc(N[C@@H]4CCCNC4)ncc3F)n2nc1N1CCS(=O)(=O)CC1]

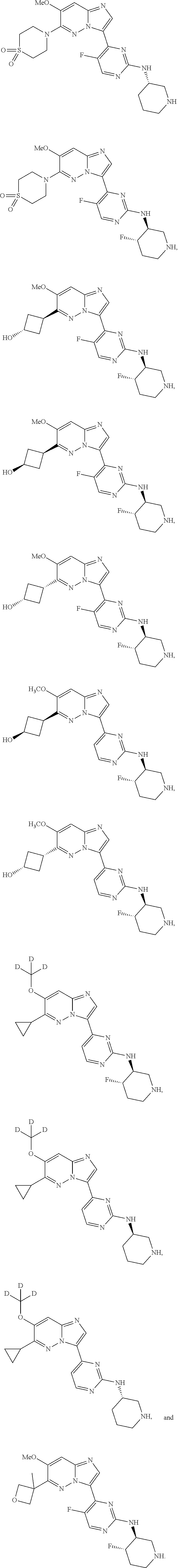

18. The compound of claim 1, wherein the compound of Formula (III) is a compound of Formula (IIIa-5010-WO50): 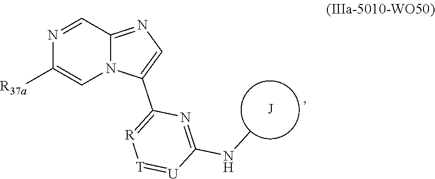
or a salt, ester, solvate, optical isomer, geometric isomer, or salt of an isomer thereof;
wherein:
R is N or CR₃₁;
T is N or CR₃₂;
U is N or CR₃₃; 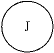
is 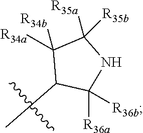
R₃₇ₐ is selected from halogen, C₁-C₆ alkyl, C₁-C₆ alkoxy, C₃-C₆ cycloalkyl, —O—(C₃-C₆ cycloalkyl), C₂-C₆ heterocyclyl, imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH, deuterium, and halogen, and wherein C₃-C₆ cycloalkyl, —O—(C₃-C₆ cycloalkyl), and C₂-C₆ heterocyclyl are each optionally substituted with one or more substituents selected from —OH, C₁-C₆ alkyl, and halogen;
R₃₁, R₃₂, and R₃₃ are each independently selected from H, C₁-C₆ alkoxy, and halogen;
R₃₄ₐ, R_{34b}, R₃₅ₐ, R_{35b}, R₃₆ₐ, and R_{36b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms;
R₃₈ₐ and R_{38b} are each independently selected from H, C₁-C₆ alkyl, and C₃-C₆ cycloalkyl, wherein C₁-C₆ alkyl and C₃-C₆ cycloalkyl are each optionally substituted with one or more halogen; and
one of R, T, or U is N.

19. The compound of claim 18, wherein at least one of (i)-(iv) applies:
(i) each of R_{34b}, R₃₅ₐ, R_{35b}, R₃₆ₐ, and R_{36b} is H and R₃₄ₐ is F;
(ii) R₃₁, R₃₂, and R₃₃, if present, are H;
(iii) R is CR₃₁ wherein R₃₁ is F, T is CR₃₂ wherein R₃₂ is H, and U is N; and
(iv) R₃₇ₐ is selected from

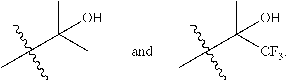

20. The compound of claim 18 or 19, with the proviso that R₃₇ₐ is not

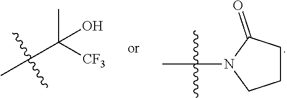

21. The compound of any one of claims 18 to 20, wherein the compound of Formula (IIIa-5010-WO50) is:

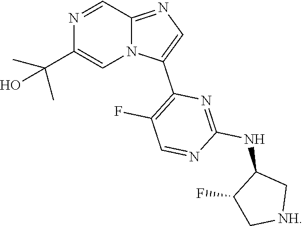 [SMILES: CC(C)(O)c1cn2c(-c3nc(N[C@H]4CNC[C@@H]4F)ncc3F)cnc2cn1]

22. The compound of claim 1, wherein the compound of Formula (III) is a compound of Formula (IIIb-5010-WO50): 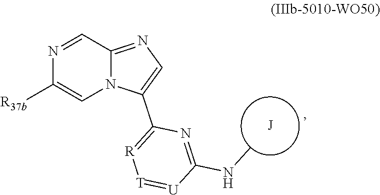
or a salt, ester, solvate, optical isomer, geometric isomer, or salt of an isomer thereof;
wherein:
R is N or CR₃₁;
T is N or CR₃₂;
U is N or CR₃₃; 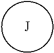
is 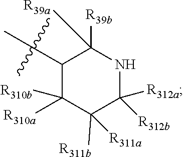
R_{37b} is selected from halogen, C₁-C₆ alkyl, C₁-C₆ alkoxy, C₃-C₆ cycloalkyl, —O—(C₃-C₆ cycloalkyl), C₂-C₆ heterocyclyl, imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH, deuterium, and halogen, and wherein C₃-C₆ cycloalkyl, —O—(C₃-C₆cycloalkyl), and C₂-C₆ heterocyclyl are each optionally substituted with one or more substituents selected from —OH, C₁-C₆ alkyl, and halogen;
R₃₁, R₃₂, and R₃₃ are each independently selected from H, C₁-C₆ alkoxy, and halogen;
R₃₉ₐ, R_{39b}, R₃₁₀ₐ, R_{310b}, R₃₁₁ₐ, R_{311b}, R₃₁₂ₐ, and R_{312b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms;
R₃₈ₐ and R_{38b} are each independently selected from H, C₁-C₆ alkyl, and C₃-C₆ cycloalkyl, wherein C₁-C₆ alkyl and C₃-C₆ cycloalkyl are each optionally substituted with one or more halogen; and
one of R, T, or U is N.

23. The compound of claim 22, wherein at least one of (i)-(v) applies:
(i) each of R₃₉ₐ, R_{39b}, R₃₁₀ₐ, R_{310b}, R₃₁₁ₐ, R_{311b}, R₃₁₂ₐ, and R_{312b} is H;
(ii) each of R₃₉ₐ, R_{39b}, R_{310b}, R₃₁₁ₐ, R_{311b}, R₃₁₂ₐ, and R_{312b} is H and R₃₁₀ₐ is F;
(iii) R₃₁, R₃₂, and R₃₃, if present, are H;
(iv) R is CR₃₁ wherein R₃₁ is F, T is CR₃₂ wherein R₃₂ is H, and U is N; and
(v) R_{37b} is selected from

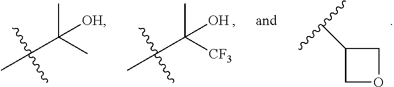

24. The compound of claim 22 or 23, with the proviso that:
when R_{37b} is 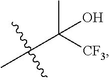 [SMILES: *C(C)(O)C(F)(F)F]
at least one of R₃₁, R₃₂, and R₃₃ is present and selected from C₁-C₆ alkoxy and halogen.

25. The compound of any one of claims 22 to 24, wherein the compound of Formula (IIIb-5010-WO50) is selected from:

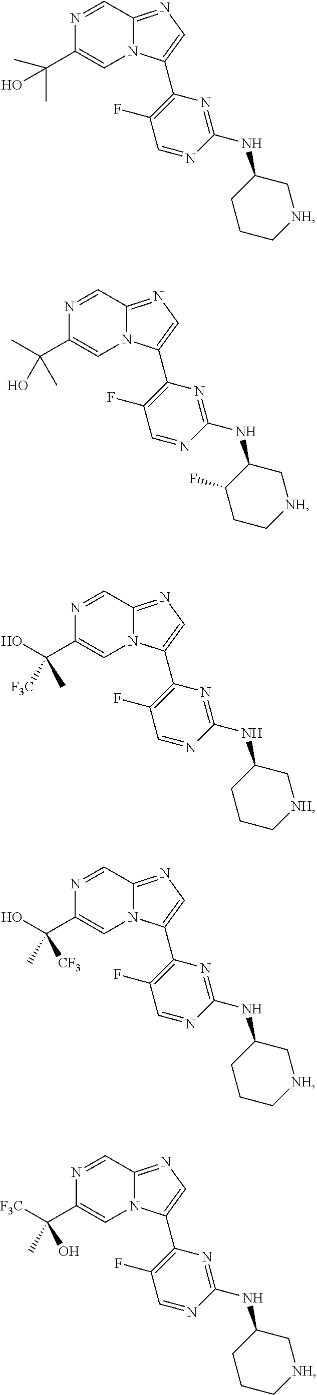 [SMILES: CC(C)(O)c1cn2c(-c3nc(N[C@@H]4CCCNC4)ncc3F)cnc2cn1 ; CC(C)(O)c1cn2c(-c3nc(N[C@H]4CNCC[C@@H]4F)ncc3F)cnc2cn1 ; C[C@@](O)(c1cn2c(-c3nc(N[C@@H]4CCCNC4)ncc3F)cnc2cn1)C(F)(F)F ; C[C@](O)(c1cn2c(-c3nc(N[C@@H]4CCCNC4)ncc3F)cnc2cn1)C(F)(F)F ; C[C@@](O)(c1cn2c(-c3nc(N[C@@H]4CCCNC4)ncc3F)cnc2cn1)C(F)(F)F]

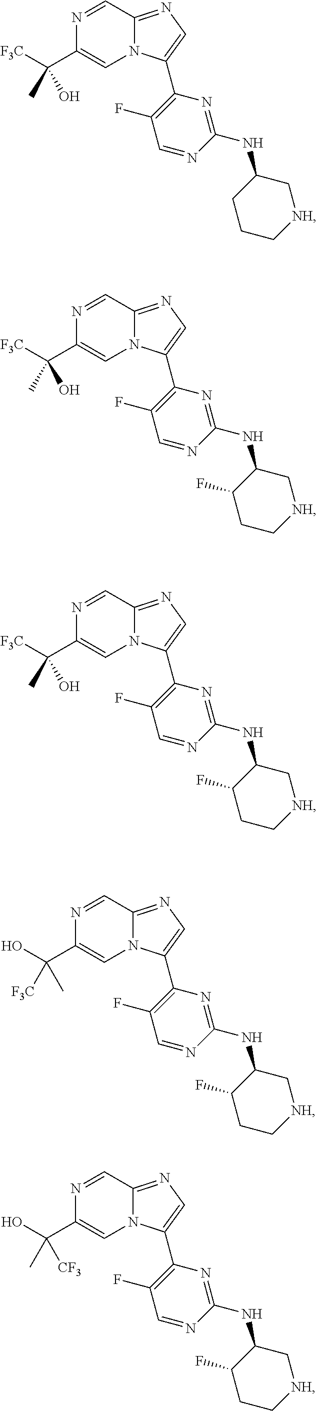 [SMILES: C[C@](O)(c1cn2c(-c3nc(N[C@@H]4CCCNC4)ncc3F)cnc2cn1)C(F)(F)F ; C[C@@](O)(c1cn2c(-c3nc(N[C@H]4CNCC[C@@H]4F)ncc3F)cnc2cn1)C(F)(F)F ; C[C@](O)(c1cn2c(-c3nc(N[C@H]4CNCC[C@@H]4F)ncc3F)cnc2cn1)C(F)(F)F ; CC(O)(c1cn2c(-c3nc(N[C@H]4CNCC[C@@H]4F)ncc3F)cnc2cn1)C(F)(F)F ; CC(O)(c1cn2c(-c3nc(N[C@H]4CNCC[C@@H]4F)ncc3F)cnc2cn1)C(F)(F)F]

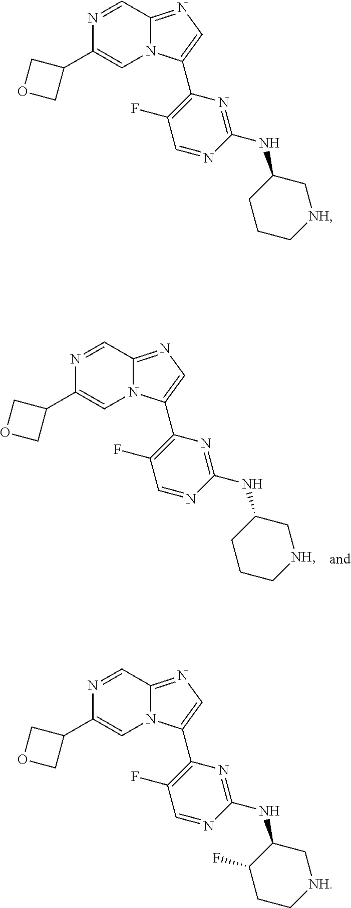 [SMILES: Fc1cnc(N[C@@H]2CCCNC2)nc1-c1cnc2cnc(C3COC3)cn12 ; Fc1cnc(N[C@H]2CCCNC2)nc1-c1cnc2cnc(C3COC3)cn12 ; Fc1cnc(N[C@H]2CNCC[C@@H]2F)nc1-c1cnc2cnc(C3COC3)cn12]

26. The compound of any one of claims 1 to 25, wherein:
(i) the compound is an inhibitor of at least one of IRAK1, IRAK4, and FLT3;
(ii) the compound is an inhibitor of IRAK1 and IRAK4 and is not an inhibitor of FLT3; or
(iii) the compound is an inhibitor of each of IRAK1, IRAK4, and FLT3.

27. A composition comprising a compound of any one of claims 1 to 26, wherein the composition further comprises a formulary ingredient, an adjuvant, or a carrier.

28. A method of treating a disease or disorder in a subject in need thereof, the method comprising administering to the subject a therapeutically effective amount of a compound of any one of claims 1 to 26 or a composition of claim 27.

29. The method of claim 28, wherein the disease or disorder is alleviated by inhibition of at least one of interleukin-1 receptor-associated kinase (IRAK) inhibition and fms-like tyrosine kinase 3 (FLT3) inhibition.

30. The method of claim 28 or 29, wherein the disease or disorder comprises a hematopoietic cancer.

31. The method of claim 30, wherein the hematopoietic cancer is selected from myelodysplastic syndrome (MDS), acute myeloid leukemia (AML), lymphoma, leukemia, chronic lymphocytic leukemia (CLL), chronic myeloid leukemia (CML), acute lymphoblastic leukemia (ALL), non-Hodgkin lymphoma, Waldenstrom's macroglobulinemia, B cell lymphoma, diffuse large B-cell lymphoma (DLBCL), DLBCL with MYD88 mutation, follicular lymphoma, and marginal zone lymphoma.

32. The method of claim 30 or 31, wherein the disease or disorder is selected from:
BCL2 inhibitor resistant acute myeloid leukemia (AML),
BCL2 inhibitor resistant refractory acute myeloid leukemia,
BCL2 inhibitor resistant relapsed acute myeloid leukemia,
FLT3 inhibitor resistant acute myeloid leukemia,
FLT3 inhibitor resistant refractory acute myeloid leukemia, or
FLT3 inhibitor resistant relapsed acute myeloid leukemia.

33. The method of claim 28 or 29, wherein the disease or disorder comprises at least one cancer selected from bone marrow cancer, glioblastoma multiforme, endometrial cancer, melanoma, prostate cancer, lung cancer, breast cancer, kidney cancer, bladder cancer, basal cell carcinoma, thyroid cancer, squamous cell carcinoma, neuroblastoma, ovarian cancer, renal cell carcinoma, hepatocellular carcinoma, colon cancer, pancreatic cancer, rhabdomyosarcoma, meningioma, gastric cancer, Glioma, oral cancer, nasopharyngeal carcinoma, rectal cancer, stomach cancer, and uterine cancer.

34. The method of claim 28 or 29, wherein the disease or disorder comprises at least one inflammatory disease or autoimmune disease selected from chronic inflammation, sepsis, rheumatoid arthritis, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, colitis, and atopic dermatitis.

35. The method of any one of claims 28 to 34, further comprising administering to the subject one or more additional therapies selected from: a chemotherapy agent, a BCL2 inhibitor, an immune modulator, a BTK inhibitor, a DNA methyltransferase inhibitor/hypomethylating agent, an anthracycline, a histone deacetylase (HDAC) inhibitor, a purine nucleoside analogue (antimetabolite), an isocitrate dehydrogenase 1 or 2 (IDH1 and/or IDH2) inhibitor, an antibody-drug conjugate, an mAbs/immunotherapy, a Plk inhibitor, a MEK inhibitor, a CDK inhibitor, a CDK9 inhibitor, a CDK8 inhibitor, a retinoic acid receptor agonist, a TP53 activator, a CELMoD, a smoothened receptor antagonist, an ERK inhibitor including an ERK2/MAPK1 or ERK1/MAPK3 inhibitor, a PI3K inhibitor, an mTOR inhibitor, a steroid or glucocorticoid, a steroid or glucocorticoid receptor modulator, an EZH2 inhibitor, a hedgehog (Hh) inhibitor, a Topoisomerase I inhibitor, a Topoisomerase II inhibitor, an aminopeptidase/Leukotriene A4 hydrolase inhibitor, a FLT3/Axl/ALK inhibitor, a FLT3/KIT/PDGFR, PKC, and/or KDR inhibitor, a Syk inhibitor, an E-selectin inhibitor, an NEDD8-activator, an MDM2 inhibitor, a PLK1 inhibitor, an Aura A inhibitor, an aurora kinase inhibitor, an EGFR inhibitor, an AuroraB/C/VEGFR1/2/3/FLT3/CSF-1R/Kit/PDGFRA/B inhibitor, an AKT 1, 2, and/or 3 inhibitor, a ABL1/2/SRC/EPHA2/LCK/YES1/KIT/PDGFRB/FYN inhibitor, a farnesyltransferase inhibitor, a BRAF/MAP2K1/MAP2K2 inhibitor, a Menin-KMT2A/MLL inhibitor, and a multikinase inhibitor.

36. The method of claim 35, wherein the additional therapy is at least one of a BCL2 inhibitor, a BTK inhibitor, a glucocorticoid, a CDK inhibitor, and a DNA methyltransferase inhibitor.

37. The method of claim 36, wherein the BCL2 inhibitor is venetoclax or a pharmaceutically acceptable salt thereof, the BTK inhibitor is ibrutinib or a pharmaceutically acceptable salt thereof, the glucocorticoid is selected from dexamethasone, methylprednisolone, prednisolone, or a pharmaceutically acceptable salt of any one thereof, the CDK inhibitor is selected from CDK4/6 inhibitor palbociclib, CDK7 inhibitor THZ1, and/or CDK9 inhibitors BAY1251152 and atuveciclib, or a pharmaceutically acceptable salt of any one thereof, or the DNA methyltransferase inhibitor is azacitidine or a pharmaceutically acceptable salt thereof.

38. The method of claim 35, wherein the compound of any one of claims 1 to 26 or the composition of claim 27 and the one or more additional therapies are administered together in one administration or composition.

39. The method of claim 35, wherein the compound of any one of claims 1 to 26 or the composition of claim 27 and the one or more additional therapies are administered separately in more than one administration or more than one composition.

40. The method of any one of claims 28 to 39, wherein the compound is a compound of any one of Formula (Ib-5010-WO50), Formula (Id-5010-WO50), Formula (IIa-5010-WO50), Formula (IIb-5010-WO50), Formula (IIIa-5010-WO50), Formula (IIIb-5010-WO50), or a salt, ester, solvate, optical isomer, geometric isomer, or salt of an isomer of any one thereof.

41. A method of increasing survivability in a subject diagnosed with acute myeloid leukemia (AML) or suspected of having AML, the method comprising administering to the subject a therapeutically effective amount of a compound of any one of claims 1 to 26 or a composition of claim 27.

42. The method of claim 41, wherein the survivability of the subject is increased compared to a subject treated with a therapeutically effective amount of the standard of care for AML.

43. The method of claim 42, wherein the standard of care for AML comprises gilteritinib or a pharmaceutically acceptable salt thereof.

44. The method of any one of claims 41 to 43, wherein the subject is a human.

45. The method of claim 44, wherein the survivability of the subject is increased by about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, or about 20 years compared to a subject treated with a therapeutically effective amount of the standard of care for AML.

46. The method of any one of claims 41 to 45, comprising administering to the subject the therapeutically effective amount of a compound of any one of claims 1 to 26 or the composition of any one of claims 27 to 30 about every 6 hours, every 12 hours, every 18 hours, once a day, every other day, every 3 days, every 4 days, every 5 days, every 6 days, or once a week.

47. The method of any one of claims 41 to 46, further comprising administering to the subject one or more additional therapies selected from: a chemotherapy agent, a BCL2 inhibitor, an immune modulator, a BTK inhibitor, a DNA methyltransferase inhibitor/hypomethylating agent, an anthracycline, a histone deacetylase (HDAC) inhibitor, a purine nucleoside analogue (antimetabolite), an isocitrate dehydrogenase 1 or 2 (IDH1 and/or IDH2) inhibitor, an antibody-drug conjugate, an mAbs/immunotherapy, a Plk inhibitor, a MEK inhibitor, a CDK inhibitor, a CDK9 inhibitor, a CDK8 inhibitor, a retinoic acid receptor agonist, a TP53 activator, a CELMoD, a smoothened receptor antagonist, an ERK inhibitor including an ERK2/MAPK1 or ERK1/MAPK3 inhibitor, a PI3K inhibitor, an mTOR inhibitor, a steroid or glucocorticoid, a steroid or glucocorticoid receptor modulator, an EZH2 inhibitor, a hedgehog (Hh) inhibitor, a Topoisomerase I inhibitor, a Topoisomerase II inhibitor, an aminopeptidase/Leukotriene A4 hydrolase inhibitor, a FLT3/Axl/ALK inhibitor, a FLT3/KIT/PDGFR, PKC, and/or KDR inhibitor, a Syk inhibitor, an E-selectin inhibitor, an NEDD8-activator, an MDM2 inhibitor, a PLK1 inhibitor, an Aura A inhibitor, an aurora kinase inhibitor, an EGFR inhibitor, an AuroraB/C/VEGFR1/2/3/FLT3/CSF-1R/Kit/PDGFRA/B inhibitor, an AKT 1, 2, and/or 3 inhibitor, a ABL1/2/SRC/EPHA2/LCK/YES1/KIT/PDGFRB/FYN inhibitor, a farnesyltransferase inhibitor, a BRAF/MAP2K1/MAP2K2 inhibitor, a Menin-KMT2A/MLL inhibitor, and a multikinase inhibitor.

48. The method of claim 47, wherein the additional therapy is at least one of a BCL2 inhibitor, a BTK inhibitor, a glucocorticoid, a CDK inhibitor, and a DNA methyltransferase inhibitor.

49. The method of claim 48, wherein the BCL2 inhibitor is venetoclax or a pharmaceutically acceptable salt thereof, the BTK inhibitor is ibrutinib or a pharmaceutically acceptable salt thereof, the glucocorticoid is selected from dexamethasone, methylprednisolone, prednisolone, or a pharmaceutically acceptable salt of any one thereof, the CDK inhibitor is selected from CDK4/6 inhibitor palbociclib, CDK7 inhibitor THZ1, and/or CDK9 inhibitors BAY1251152 and atuveciclib, or a pharmaceutically acceptable salt of any one thereof, and the DNA methyltransferase inhibitor is azacitidine or a pharmaceutically acceptable salt thereof.

50. The method of any one of claims 41 to 49, wherein the AML is selected from:
BCL2 inhibitor resistant AML,
BCL2 inhibitor resistant refractory AML,
BCL2 inhibitor resistant relapsed AML,
FLT3 inhibitor resistant AML,
FLT3 inhibitor resistant refractory AML, or
FLT3 inhibitor resistant relapsed AIL.

51. The method of claim 47, wherein the compound of any one of claims 1-26 or the composition of claim 27 and the one or more additional therapies are administered together in one administration or composition.

52. The method of claim 47, wherein the compound of any one of claims 1-26 or the composition of claim 27 and the one or more additional therapies are administered separately in more than one administration or more than one composition.
